(19) United States
(12) Patent Application Publication
(10) Pub. No.: US 2026/0184814 A1
(43) Pub. Date: Jul. 2, 2026

(54) BROADLY NEUTRALIZING ANTIBODIES AGAINST INFLUENZA NEURAMINIDASE

(71) Applicants: Humabs BioMed SA, Bellinzona (CH); Vir Biotechnology, Inc., San Francisco, CA (US)

(72) Inventors: Elisabetta Cameroni, Claro (CH); Davide Corti, Bellinzona (CH); Kevin E. Hauser, Jupiter, FL (US); Corey Momont, San Francisco, CA (US); Matteo Samuele Pizzuto, Massagno (CH); Gyorgy Snell, San Francisco, CA (US)

(21) Appl. No.: 18/868,143

(22) PCT Filed: May 22, 2023
(86) PCT No.: PCT/US2023/067312

§ 371 Date: Nov. 21, 2024

Related U.S. Application Data
(60) Provisional application No. 63/345,016, filed on May 23, 2022.
(60) Provisional application No. 63/427,799, filed on Nov. 23, 2022.
(60) Provisional application No. 63/430,319, filed on Dec. 5, 2022.
(60) Provisional application No. 63/481,977, filed on Jan. 27, 2023.
(60) Provisional application No. 63/488,433, filed on Mar. 3, 2023.
(60) Provisional application No. 63/493,057, filed on Mar. 30, 2023.

(51) Int. Cl. C07K 16/40; A61K 39/00; A61P 37/04

(52) U.S. Cl. CPC C07K 16/40; A61P 37/04; A61K 2039/505; A61K 2039/545; C07K 2317/52; C07K 2317/76; C07K 2317/92; C07K 2317/94

(57) ABSTRACT

The instant disclosure provides antibodies and antigen-binding fragments thereof that can bind to an influenza virus neuraminidase (NA) and can neutralize an influenza virus infection. Also provided are polynucleotides that encode an antibody, vectors that comprise such polynucleotides, host cells that can express the antibodies, related compositions, and methods of using the herein disclosed compositions to, for example, treat or prevent an influenza infection.

### REFERENCE TO AN ELECTRONIC SEQUENCE LISTING

This application contains a Sequence Listing, which has been submitted electronically in xml format and is hereby incorporated by reference in its entirety. Said xml copy, created on Feb. 11, 2025, is named SeqList-368564-43901.xml and is 146,471 bytes in size.

### BACKGROUND

Influenza is an infectious disease which spreads around the world in yearly outbreaks, resulting per year in about three million to about five million cases of severe illness and about 290,000 to 650,000 respiratory deaths (WHO, Influenza (Seasonal) Fact sheet, Nov. 6, 2018). The most common symptoms include: a sudden onset of fever, cough (usually dry), headache, muscle and joint pain, severe malaise (feeling unwell), sore throat and a runny nose. The incubation period varies between one to four days, although usually symptoms begin about two days after exposure to the virus. Complications of influenza may include pneumonia, sinus infections, and worsening of previous health problems such as asthma or heart failure, sepsis or exacerbation of chronic underlying disease.

Influenza is caused by influenza virus, an antigenically and genetically diverse group of viruses of the family Orthomyxoviridae that contains a negative-sense, single-stranded, segmented RNA genome. Of the four types of influenza virus (A, B, C and D), three types (A, B and C) are known to affect humans. Influenza viruses can be categorized based on the different subtypes of major surface proteins present: Hemagglutinin (HA) and Neuraminidase (NA). There are at least 18 influenza A subtypes defined by their hemagglutinin (“HA”) proteins. The HAs can be classified into two groups. Group 1 includes H1, H2, H5, H6, H8, H9, H11, H12, H13, H16 and H17 subtypes, and group 2 includes H3, H4, H7, H10, H14 and H15 subtypes. There are at least 11 different neuraminidase subtypes (N1 through N11, respectively (cdc.gov/flu/about/viruses/types.htm)). Neuraminidases function in viral mobility and spread by catalyzing hydrolysis of sialic acid residues on virions prior to release from an infected host cell, and on target cell surface glycoproteins. Drugs designed to inhibit neuraminidase (NAIs) have been developed (e.g., oseltamivir, zanamivir, peramivir, laninamivir), though naturally acquired mutations of IAV subtypes have reduced susceptibility to current NAIs (Hussain et al., Infection and Drug Resistance 10:121-134 (2017).

New modalities for treating influenza virus infections are needed.

### BRIEF DESCRIPTION OF THE DRAWINGS

FIG. 1 shows a workflow for anti-“NA” (neuraminidase) monoclonal antibody discovery. Donors were selected by screening serum from (human) tonsillar donor samples (n=50) for reactivity against neuraminidase subtype N1 and N2 antigens, and serum from PBMC (peripheral blood mononuclear cell) donor samples (n=124) for reactivity against neuraminidase subtypes N4, N3, and N9. Neuraminidase antigens for screening were expressed in mammalian cells and binding was evaluated by flow cytometry. B memory cells from five donors were sorted by flow cytometry for input into the discovery workflow. Single sorted B cells (n=39,350) were co-cultured with mesenchymal stromal cells (MSC) in 50 μl cultures to stimulate antibody secretion. Secreted antibodies were evaluated by binding and NA inhibition assays. Inhibition of N1 sialidase activity was evaluated using ELLA (enzyme-linked lectin assay), and inhibition of N1, N2, and N9 sialidase activity was measured using a fluorescence-based assay that measures cleavage of the 2′-(4-Methylumbelliferyl)-α-D-N-acetylneuraminic acid (MUNANA). “NI activity” refers to neuraminidase inhibition activity. Binding to NAs from group 1 IAV N1 A/Vietnam/1203/2004, and group 2 IAVs N2 A/Tanzania/205/2010 and N9 A/Hong Kong/56/2015 was evaluated by ELISA to determine breadth. Antibody sequences from selected B cells were cloned as cDNAs and sequenced.

FIG. 2A shows VH domain sequence alignments of monoclonal antibodies (with “FNI” prefix) against Influenza A Viruses (“IAV”) that were isolated from human donor PBMCs. FIG. 2B shows VH domain sequence alignments of “FNI3” and “FNI9” (FNI9 comprises the following VH and VL amino acid sequences: VH: SEQ ID NO:2; VL: SEQ ID NO.: 8) with the unmutated common ancestor, “UCA” (VH: SEQ ID NO:98; VL: SEQ ID NO:100).

FIGS. 3A-3C show binding of FNI3 and FNI9 to N1 (FIG. 3A), N2 (FIG. 3B), and N9 (FIG. 3C) NAs measured by enzyme-linked immunosorbent assay (ELISA), reported as OD versus concentration in ng/ml. Binding by a comparator antibody, 1G01-LS, and a negative control antibody against an irrelevant antigen, “K—” was also measured.

FIGS. 4A-4C show binding kinetics of FNI3 bearing M428L/N434S Fc mutations (“FNI3-LS” in the figures) and FNI9 bearing M428L/N434S Fc mutations (“FNI9-LS” in the figures) to N1 (FIG. 4A), N2 (FIG. 4B), and N9 (FIG. 4C) NAs, as measured by Bio-Layer Interferometry (BLI). Dissociation is reported as kdis (1/s), association is reported as kon (1/Ms), and KD was calculated from the ratio of kdis/kon. Binding by a comparator antibody, 1G01-LS, was also measured.

FIG. 5 summarizes results from flow cytometry assays testing binding by FNI3 and FNI9, as well as by comparator antibody 1G01, against a panel of group I IAV, group II IAV, and Influenza B Virus (IBV) NAs. Bold font indicates NAs from influenza viruses isolated from humans. Values on the scale at right show range of calculated EC50. Values were selected based on the lowest concentration at which binding was observed.

FIGS. 6A-6C relate to activity of FNI3 and FNI9 against NAs that bear a glycosylation site. FIG. 6A shows glycosylation sites of group 2 IAV N2 subtype NAs at positions 245 (245Gly+) and 247 (247Gly+) in A/South Australia/34/2019, A/Switzerland/8060/2017, A/Singapore/INFIMH-16-0019/2016, and A/Switzerland/9715293/2013. FIG. 6B summarizes inhibition of sialidase activity (NAI) in A/Switzerland/8060/2017, A/Singapore/INFIMH-16-0019/2016, and A/Switzerland/9715293/2013 live virus stocks, reported as EC50 in μg/ml. FIG. 6C shows binding of FNI3 and FNI9 to NA in mammalian cells infected with A/South Australia/34/2019 (245Gly+) measured by flow cytometry. Mock staining is shown as a negative control.

FIG. 7 shows binding of FNI3 and FNI9 to NA expressed on mammalian cells infected with a H1N1 Swine Eurasian avian-like (EA) strain, A/Swine/Jiangsu/J004/2018, measured by flow cytometry. Mock staining is shown as a negative control.

Another experiment (data not shown) showed lack of polyreactivity of FNI3 and FNI9 binding against human epithelial type 2 (HEP-2) cells. Anti-HA antibody FI6v3 was used as a positive control, and anti-paramyxovirus antibody MPE8 was included as a negative control.

FIG. 8 summarizes inhibition of sialidase activity (“NAI”) by FNI3 and FNI9 against a panel of group I IAV, group II IAV, and Influenza B Virus (IBV) NAs, as measured by MUNANA assay.

FIG. 9 shows in vitro inhibition of sialidase activity (reported as IC50 in μg/ml) by FNI3 and FNI9 against group I (H1N1) IAV, group II (H3N2) IAV, and IBV NAs.

FIGS. 10A-10B show in vitro inhibition of sialidase activity (reported as IC50 in μg/ml) by FNI3 and FNI9 against group I (H1N1) IAV, group II (H3N2) IAV, and IBV NAs. FIG. 10A depicts inhibition activity against group I IAVs, group II IAVs, and IBVs within the same plot and FIG. 10B depicts against these IAVs in separate plots.

FIG. 11A shows a panel of IAV and IBV strains tested in an in vitro inhibition of sialidase activity assay. FIG. 11B shows results from the assay (reported as IC50 in μg/ml) for FNI3, FNI9, FNI14, FNI17, and FNI19. Asterisk in figure key indicates a glycosylation site is present in position 245.

FIGS. 12A-12D show neutralization of antibodies FNI1, FNI3, FNI9, FNI14, FNI17, and FNI19 against H1N1 A/California/07/2009 (FIG. 12A), H3N2 A/Hong Kong/8/68 (FIG. 12B), B/Malaysia/2506/2004 (FIG. 12C), and B/Jiangsu/10/2003 (FIG. 12D) NAs (reported as IC50 in μg/ml).

FIGS. 13A and 13B show antibody activation of FcγRIIIa (FIG. 13A; F158 allele) and FcγRIIa (FIG. 13B; H131 allele). Activation was measured using an NFAT-mediated Luciferase reporter in engineered Jurkat cells. FNI3 and FNI9 were tested, along with a comparator antibody FM08 (“FM08 LS” in the figure; VH: SEQ ID NO:25; VL: SEQ ID NO:26) and a negative control antibody (FY1-GRLR).

FIGS. 14A and 14B show frequency by year of NA antiviral-resistant mutations in (FIG. 14A) N1 (H1N1, swine H1N1, and avian H5N1) and (FIG. 14B) N2 (H3N2, H2N2) subtypes.

FIGS. 15A to 15E show neutralization of H1N1 A/California/07/2009 virus engineered with reverse genetics to harbor oseltamivir (OSE)-resistant mutations (H275Y, E119D and H275Y, or S247N and H275Y) by anti-flu antibodies or oseltamivir. Neutralization activity of FNI3 (FIG. 15A), FNI9 (FIG. 15B), and oseltamivir (FIG. 15C) were measured, along with comparator antibodies FM08 (FIG. 15D) and 1G01 (FIG. 15E).

FIGS. 16A and 16B show neutralization of group I (H1N1) IAV, group II (H3N2) IAV, IBV viruses, and IAV and IBV viruses engineered with reverse genetics to harbor OSE-resistant mutations (H275Y, E119D/H275Y, H275Y/S247N, I222V, or N294S), by anti-NA antibodies (reported as IC50 in μg/ml). Asterisks in FIG. 16A (x-axis) indicate viruses bearing OSE-resistant mutations. Neutralization activity of FNI3, FNI9, and a comparator antibody, 1G01, was measured. FIG. 16A depicts neutralization of individual viral strains and FIG. 16B depicts neutralization of viral strains grouped by neutralizing anti-NA antibody.

FIG. 17 shows data from crystal structure studies showing docking of the antigen-binding fragment (Fab) domain of the FNI3 antibody with NA.

FIGS. 18A and 18B show diagrams constructed from crystal structure studies of the heavy chain complementarity-determining region 3 (H-CDR3) of the FNI3 heavy chain when it is unbound (FIG. 18A) or bound to N2 NA (FIG. 18B). The unbound FNI3 H-CDR3 crystal structure (FIG. 18A) shows a beta sheet conformation and intact main chain hydrogen bonds between carboxylic acid groups (CO) and amino groups (NH) of residues E111 (CO)-D102 (NH), E111 (NH)-D102 (CO), G109 (CO)-F104 (NH), G109 (NH)-N105 (CO), and L108 (NH)-N105 (CO). The FNI3-N2 crystal structure (FIG. 18B) shows disruption of the H-CDR3 beta sheet conformation and one intact main chain hydrogen bond between G109 (CO)-F104 (NH).

FIGS. 19A and 19B show diagrams generated from crystal structure studies showing angle of docking of the antigen-binding fragment (Fab) domain of FNI3 and of comparator antibodies 1G01, 1G04, and 1E01, in complex with NA subtypes. Lines indicate angle of docking in all panels and Protein Data Bank (PDB) identification codes are shown for comparator antibodies 1G01, 1G04, and 1E01. FIG. 19A shows 1G01 in complex with N1 NA (upper panel) and 1G04 in complex with N9 NA (lower panel). FIG. 19B shows FNI3 in complex with N2 NA (upper panel) and 1E01 in complex with N2 NA (lower panel).

FIG. 20 shows conformation and interactions of FNI3 CDRs: H-CDR3, H-CDR2, and L-CDRs. To generate these data, proteins were “quick prepped” using MOE (Molecular Operating Environment).

FIG. 21 shows crystal structure of FNI3 in complex with N2 NA, including residues of light chain CDRs (L-1, L-2, L-3) and heavy chain CDRs (H-1, H-2, H-3). The interaction of H-CDR3 with N2 NA is shown in enhanced resolution in the right panel. Negative numbers are interaction energy in kcal/mol. Proteins were “quick prepped” using MOE (Molecular Operating Environment) software.

FIG. 22 shows a crystal structure representation of FNI3 in complex with oseltamivir-bound N2 NA. Oseltamivir is shown interacting with R292, R371, and R118 of N2 NA.

FIG. 23 shows an alternative view of the crystal structure showing FNI3 in complex with oseltamivir-bound N2 NA.

FIGS. 24A and 24B show analysis of FNI3 epitope conservation in N2 NA sequences from H3N2 (n=60, 597) isolated between the years 2000 and 2020. The table in FIG. 24A shows frequency of an amino acid at a particular position in the analyzed N2 NA sequences. Circled values indicate amino acids appearing at the lowest three frequencies, Glu221 (E221, 17.41%), Ser245 (S245, 33.69%), and Ser247 (S247, 36.16%). Acidic amino acids include: aspartic acid, glutamic acid; basic amino acids include: arginine, histidine, lysine; hydrophobic amino acids include: isoleucine, leucine, tryptophan, valine, alanine, proline; neutral amino acids include: asparagine, glutamine; and polar amino acids include: serine, threonine, glycine, tyrosine. FIG. 24B shows interaction of VH Y60 and Y94 from FNI3 with E221, S245, and S247 of N2 NA.

FIG. 25 shows a comparison of N2 NA FNI3 epitope conservation (top; as shown in FIGS. 24A and 24B) with FNI3 epitope conservation in N1 NA sequences from H1N1 (n=57,597) isolated between the year 2000 and 2020 (bottom). Acidic amino acids include: aspartic acid, glutamic acid; basic amino acids include: arginine, histidine, lysine; hydrophobic amino acids include: isoleucine, leucine, tryptophan, valine, alanine, proline; neutral amino acids include: asparagine, glutamine; and polar amino acids include: serine, threonine, glycine, tyrosine.

FIGS. 26A and 26B show the design of an in vivo study to evaluate prophylactic activity of FNI3 (“mAb-03” in FIG. 26A) and FNI9 (“mAb-09” in FIG. 26A) in BALB/c mice infected with IAV A/Puerto Rico/8/34 or A/Hong Kong/8/68. FIG. 26A shows the dosing and virus strains used in the study. FIG. 26B shows the timeline and endpoints of the study.

FIGS. 27A-27D show measurements of body weight over fifteen days in BALB/c mice infected with H1N1 A/Puerto Rico/8/34 following pre-treatment with FNI9. Antibody was administered at 6 mg/kg (FIG. 27A), 2 mg/kg (FIG. 27B), 0.6 mg/kg (FIG. 27C), or 0.2 mg/kg (FIG. 27D), one day prior to infection with a LD90 (90% lethal dose) of A/Puerto Rico/8/34. Body weight of mice administered a vehicle control was also measured (left graph in each figure).

FIGS. 28A-28D show measurements of body weight over fifteen days in BALB/c mice infected with H3N2 A/Hong Kong/8/68 following pre-treatment with FNI9. Antibody was administered at 6 mg/kg (FIG. 28A), 2 mg/kg (FIG. 28B), 0.6 mg/kg (FIG. 28C), or 0.2 mg/kg (FIG. 28D), one day prior to infection with a LD90 (90% lethal dose) of A/Hong Kong/8/68. Body weight of mice receiving a vehicle control was also measured (left graph in each figure).

FIGS. 29A and 29B show survival over fifteen days in BALB/c mice infected with A/Puerto Rico/8/34 (FIG. 29A) or A/Hong Kong/8/68 (FIG. 29B) following treatment with FNI3 or FNI9. Survival in mice pre-treated with a vehicle control was also measured.

FIGS. 30A and 30B show body weight loss from day 4 to 14 post-infection (reported as area-under-the-curve) in BALB/c mice infected with A/Puerto Rico/8/34 (FIG. 30A) or A/Hong Kong/8/68 (FIG. 30B) following pre-treatment with FNI3 or FNI9. Body weight loss in mice pre-treated with a vehicle control was also measured.

FIGS. 31A and 31B show negative area-under-the-curve peak values compared with IgG in serum from area-under-the-curve analysis of body weight loss in BALB/c mice infected with A/Puerto Rico/8/34 (FIG. 31A) or A/Hong Kong/8/68 (FIG. 31B) following treatment with FNI3 or FNI9.

FIG. 32 shows in vivo pharmacokinetics of FNI3 (“FNI3-LS”), FNI9 (“FNI9-LS”) and comparator antibodies FM08 and 1G01 (“1G01-LS”), all bearing M428L/N434S mutations, in tg32 mice. Calculated half-life is highlighted by a rectangle.

FIG. 33 summarizes results from flow cytometry assays testing binding by FNI3, FNI9, FNI17, and FNI19 at the indicated concentrations (μg/mL) against a panel of group I IAV-, group II IAV-, and Influenza B Virus (IBV) NAs transiently expressed on mammalian cells. Bold font indicates NAs from influenza viruses isolated from humans. Values on the scale at right show range of calculated EC50. Values were selected based on the lowest concentration at which binding was observed.

FIG. 34 shows in vitro inhibition of sialidase activity (reported as IC50 in μg/ml) by FNI3, FNI9, FNI17, and FNI19 against group I (H1N1) and group II (H3N2) NAs from IAVs circulating in humans. Rectangles indicate group II (H3N2) NAs harboring glycosylation at position 245 and corresponding sialidase inhibition values (reported as IC50 in μg/ml).

FIG. 35 shows in vitro inhibition of sialidase activity (reported as IC50 in μg/ml) by FNI3, FNI9, FNI17, and FNI19 against a panel of human ancestral, Victoria-lineage, and Yamagata-lineage IBV NAs.

FIG. 36 shows in vitro neutralizing activity measured by nucleoprotein (NP) staining of FNI3, FNI9, FNI17, and FNI19 against group I (H1N1) IAV, group II (H3N2) IAV, and IBV NAs. Neutralizing activity of comparator anti-HA antibodies “FM08_LS” and “FHF11v9” was also measured.

FIG. 37 shows in vitro neutralizing activity, measured by nucleoprotein (NP) staining, by FNI3, FNI9, FNI17, FNI19, and oseltamivir (OSE) against group I (H1N1) IAV, group II (H3N2) IAV, and IBV NAs. 1 nM=150 μl.

FIGS. 38A and 38B show in vitro inhibition of sialidase activity (reported as IC50 in μg/ml) by FNI3 and FNI9 against NAs from OSE-resistant influenza viruses, as measured by MUNANA assay. OSE-resistant IAVs were engineered with reverse genetics to harbor Oseltamivir (OSE)-resistant mutations. FIG. 38A shows inhibition of sialidase activity against Cal/09 N1 and Cal/09 N1 OSE-resistant (H1N1). FIG. 38B shows inhibition of sialidase activity against Aichi/68 N2 and Aichi/68 N2 OSE-resistant NAs (H3N2).

FIG. 39 shows antibody activation of FcγRIIIa (F158 allele) and FcγRIIa (H131 allele). Activation was measured using an NFAT-mediated luciferase reporter in engineered Jurkat cells. Activation was assessed following incubation with A549 cells infected with H1N1 influenza strain A/Puerto Rico/8/34 at a multiplicity of infection (MOI) of 6. FNI3, FNI9, FNI17, and FNI19 were tested, along with a comparator antibody “FM08_MLNS” bearing M428L/N434S mutations, and a negative control antibody (FY1-GRLR).

FIGS. 40A and 40B show antibody activation of FcγRIIIa (V158 allele) following incubation with IAV (FIG. 40A) and IBV (FIG. 40B) NAs. Activation was measured using an NFAT-mediated luciferase reporter in engineered Jurkat cells following incubation with Expi-CHO cells transiently transfected with plasmids encoding different IAV and IBV NAs. FNI3, FNI9, FNI17, and FNI19 were tested, along with a negative control antibody (FY1-GRLR).

FIGS. 41A and 41B show antibody activation of FcγRIIa (H131 allele) following incubation with IAV (FIG. 41A) and IBV (FIG. 41B) NAs. Activation was measured using an NFAT-mediated luciferase reporter in engineered Jurkat cells following incubation with Expi-CHO cells transiently transfected with plasmids encoding different IAV and IBV NAs. FNI3, FNI9, FNI17, and FNI19 were tested, along with a negative control antibody (FY1-GRLR).

FIG. 42 shows negative area-under-the-curve peak values (reported as IC50 in μg/ml) compared with IgG in serum from area-under-the-curve analysis of body weight loss in BALB/c mice infected with A/Puerto Rico/8/34 (H1N1) or A/Hong Kong/8/68 (H3N2) following treatment with FNI3, FNI9, or FM08_LS. For fitting purposes, the Area of Negative Peaks from the vehicle groups have been calculated at the IgG concentration of 10⁻¹ μg/ml.

FIGS. 43A and 43B show the design of an in vivo study to evaluate prophylactic activity of FNI3_MLNS (“mAb-03” in FIG. 43A) and FNI9_MLNS (“mAb-09” in FIG. 43A) in DBA/2J mice infected with IBVs B/Victoria/504/2000 (Yamagata) or B/Brisbane/60/2008 (Victoria). FIG. 43A shows the dosing and virus strains used in the study. FIG. 43B shows the timeline and endpoints of the study.

FIGS. 44A-44D show measurements of body weight over fifteen days in DBA/2 mice that were infected with IBV B/Victoria/504/2000 (Yamagata) following pre-treatment with FNI3 or FNI9. Antibody was administered at 6 mg/kg (FIG. 44A), 2 mg/kg (FIG. 44B), 0.6 mg/kg (FIG. 44C), or 0.2 mg/kg (FIG. 44D), one day prior to infection with a LD90 (90% lethal dose) of IBV B/Victoria/504/2000 (Yamagata). Body weight of mice administered a vehicle control was also measured (left graph in each figure).

FIGS. 45A-45D show measurements of body weight over fifteen days in DBA/2 mice that were infected with IBV B/Brisbane/60/2008 (Victoria) following pre-treatment with FNI3 or FNI9. Antibody was administered at 6 mg/kg (FIG. 45A), 2 mg/kg (FIG. 45B), 0.6 mg/kg (FIG. 45C), or 0.2 mg/kg (FIG. 45D), one day prior to infection with a LD90 (90% lethal dose) of IBV B/Brisbane/60/2008 (Victoria). Body weight of mice administered a vehicle control was also measured (left graph in each figure).

FIGS. 46A and 46B show body weight loss from day 4 to 14 post-infection (reported as change in weight area-under-the-curve) in DBA/2 mice infected with B/Victoria/504/2000 (Yamagata) (FIG. 46A) or B/Brisbane/60/2008 (Victoria) (FIG. 46B) following pre-treatment with FNI3 or FNI9. Body weight loss in mice pre-treated with a vehicle control was also measured.

FIGS. 47A and 47B show survival over fifteen days in DBA/2 mice infected with B/Victoria/504/2000 (Yamagata) (FIG. 47A) or B/Brisbane/60/2008 (Victoria) (FIG. 47B) following treatment with FNI3 or FNI9. Survival in mice pre-treated with a vehicle control was also measured.

FIGS. 48A and 48B show FNI3 epitope conservation in IAV and IBV NAs. FIG. 48A shows an analysis of N2 NA sequences from H1N2, H2N2, H3N2, and H5N2 IAVs (n=65,5262) (top) versus N1 NA sequences from H1N1 and H5N1 (N=58,954) (bottom). All sequences were isolated between the year 2000 and 2020. Acidic amino acids include: aspartic acid, glutamic acid; basic amino acids include: arginine, histidine, lysine; hydrophobic amino acids include: isoleucine, leucine, tryptophan, valine, alanine, proline; neutral amino acids include: asparagine, glutamine; and polar amino acids include: serine, threonine, glycine, tyrosine. Residues surrounded by squares in FIG. 48A indicate certain amino acids described in the lower panel of FIG. 48B. The table in FIG. 48B shows important FNI3-interacting residues within N2 NA and counterpart FNI3 CDRH3 residues.

FIG. 49 shows FNI3 epitope conservation in IBV NAs. IBV NA sequences from B/Brisbane/60/2008 (“FluB Victoria” in the figure; N=7,814; top) versus IBV NA sequences from B/Victoria/504/2000 (“FluB Yamagata” in the figure; N=13,243; bottom) were analyzed. Acidic amino acids include: aspartic acid, glutamic acid; basic amino acids include: arginine, histidine, lysine; hydrophobic amino acids include: isoleucine, leucine, tryptophan, valine, alanine, proline; neutral amino acids include: asparagine, glutamine; and polar amino acids include: serine, threonine, glycine, tyrosine. Residues surrounded by squares indicate primary NA residues interacting with the FNI3 HCDR3 which are 100% conserved among IAV N1/N2 and IBVs.

FIGS. 50A and 50B show in vivo pharmacokinetics of FNI antibodies bearing MLNS Fc mutations (FNI3 (“FNI3-LS”), FNI9 (“FNI9-LS”), FNI17 (“FNI17-LS”), FNI19 (“FNI19-LS”)), and comparator antibody FM08_MLNS in SCID tg32 mice over 30 days post-administration. Concentration over time (reported as μg/ml) is shown in FIG. 50A. The table in FIG. 50B shows half-life (reported in days), AUC (reported in day*μg/ml), clearance (reported in μg/ml), and volume (reported in ml).

FIG. 51 shows conservation of the top five interacting residues within the FNI NA epitope in group I IAVs, group II IAVs, and IBVs from 2009 to 2019.

FIG. 52 shows in vitro neutralizing activity measured by nucleoprotein (NP) staining by FNI9, Oseltamivir (OSE), and a comparator antibody “FM08” against H3N2 A/Hong Kong/8/68 virus. Calculated IC50 (in nM), IC80 (in nM), and maximum inhibition (reported as a percentage) are shown below the graph.

FIG. 53 shows antibody activation of FcγRIIIa (F158 allele) and FcγRIIa (H131 allele) by “GAALIE” Fc variant antibodies (comprising G236A/A330L/I332E mutations in the Fc). Activation was measured using an NFAT-mediated luciferase reporter in engineered Jurkat cells. Activation was assessed following incubation with A549 cells infected with H1N1 influenza strain A/Puerto Rico/8/34 at a multiplicity of infection (MOI) of 6. FNI3, FNI9, FNI17, and FNI19 were tested, along with FNI3, FNI9, FNI17, and FNI19 antibodies bearing GAALIE mutations (suffix “-GAALIE”). A comparator antibody “FM08_LS” and a negative control antibody (FY1-GRLR) were also tested.

FIG. 54 shows the design of an inter-experiment in vivo study to compare prophylactic activity of FM08_LS with FNI3_LS and FNI9_LS in BALB/c mice infected with IAV A/Puerto Rico/8/34 or A/Hong Kong/8/68. The table shows dosing and virus strains used in the study. The timeline and endpoints of the study are the same as those shown in FIG. 26B. Body weight data from Experiment A (“Exp-A”) are shown in FIGS. 27A-27D (FNI9-LS, A/Puerto Rico/8/34) and FIGS. 28A-28D (FNI9-LS, A/Hong Kong/8/68).

FIG. 55 shows the design of an in vivo study to evaluate biological potency of oseltamivir (OSE) in female BALB/c mice infected with IAV A/Puerto Rico/8/34. The timeline shows time of infection, OSE dosing, and endpoints of the study. OSE was administered at 10 mg/kg by oral gavage on Day 0 beginning at two hours prior to infection with 10-fold LD50 (50% lethal dose) of A/Puerto Rico/8/34. OSE was administered at the same dose at 6 hours post-infection and then twice daily until day 6 post-infection.

FIG. 56 shows measurements of body weight over fourteen days in BALB/c mice infected with H1N1 A/Puerto Rico/8/34 following pre-treatment with oseltamivir (OSE). Weight loss in mice pre-treated with a vehicle control (H2O) was also measured.

FIG. 57 shows survival over fourteen days in BALB/c mice infected with H1N1 A/Puerto Rico/8/34 following treatment with oseltamivir (OSE). Survival in mice pre-treated with a vehicle control (H2O) was also measured.

FIG. 58 shows viral titer in lung homogenates from BALB/c mice treated with OSE and infected with H1N1 A/Puerto Rico/8/34. Lung tissue was collected at two and four days post-infection. Titer is reported as 50% tissue culture infectious dose per gram tissue (TCID50/g).

FIGS. 59A-59E show in vitro inhibition of sialidase activity (reported as IC50 in μg/ml) by FNI9 and certain FNI9 variants against IAV NAs and IBV NAs. Neutralization activity of FNI9 and FNI9 variants is shown for group I (H1N1) IAV NA1 from H5N1 A/Vietnam/1203/2004 (FIG. 59A), NA2 from H3N2 A/Tanzania/205/2010 (FIG. 59B), and NA9 from H7N9 A/Hong Kong/56/2015 (FIG. 59C). Neutralization activity of FNI9 and variants is shown for BNA2 from B/Malaysia/2506/2004 (FIG. 59D) and BNA7 from B/Perth/211/2011 (FIG. 59E). In these figures, “FNI9-v6”, “FNI9-v7”, “FNI9-v8”, and “FNI9-v9” are as shown in FIG. 67 and are not the same as the FNI9 variant sequences “-VH.6”, “-VH.7”, “-VH.8”, and “-VH.9” as shown in e.g. FIGS. 70-72.

FIG. 60 shows additional characteristics of FNI9 VH. An overall surface charge map was generated using PyMOL for FNI9 along with pK values and resolution (reported in Å).

FIG. 61 shows binding energy between FNI antibodies FNI3, FNI9, FNI17, and FNI19 with highly conserved residues on NA that are involved with interacting with sialic acid.

FIG. 62 shows binding of FNI3, FNI9, FNI17, and FNI19 to NA expressed on mammalian cells infected with a H1N1 Swine Eurasian avian-like (EA) strain, A/Swine/Jiangsu/J004/2018, measured by flow cytometry. Mock antibody staining is shown as a negative control.

FIG. 63 shows binding kinetics of FNI3, FNI9, and FNI17 to N9 NA, as measured by Bio-Layer Interferometry (BLI). KD was calculated from the ratio of kdis/kon, wherein kdis is dissociation calculated as (1/s) and kon is association calculated as (1/Ms).

FIG. 64 shows in vitro inhibition of sialidase activity (reported in ng/ml) by FNI3, FNI9, FNI17, FNI17-v19, FNI19, and FNI19-v3 against group II H7N9 A/Anhui/1/2013 IAV NA.

FIG. 65 shows antibody activation of FcγRIIIa (V158 allele) following incubation with group II H7N9 A/Anhui/1/2013 IAV. Activation was measured using an NFAT-mediated luciferase reporter in engineered Jurkat cells following incubation with Expi-CHO cells transiently transfected with plasmids encoding N9 from A/Anhui/1/2013 IAV. FNI3, FNI9, FNI17, and FNI19 were tested, along with a negative control antibody (FY1-GRLR).

FIGS. 66A-66C show antibody activation of FcγRIIa (H131 allele) by “GAALIE” Fc variant antibodies (comprising G236A/A330L/I332E mutations in the Fc). Activation was measured using an NFAT-mediated luciferase reporter in engineered Jurkat cells following incubation with Expi-CHO cells transiently transfected with plasmids encoding different IAV (H1N1 A/California/07/2009 in FIG. 66A; H3N2 A/Hong Kong/8/68 in FIG. 66B) and IBV (B/Malaysia/2506/2004 in FIG. 66C) NAs. FNI3, FNI9, FNI17, and FNI19 were tested, along with FNI3, FNI9, FNI17, and FNI19 antibodies bearing GAALIE mutations (suffix “-GAALIE” in the figure). A comparator antibody “FM08_LS” and a negative control antibody (FY1-GRLR) were also tested. FM08_LS and FY1-GRLR had the lowest measured values in FIGS. 66A-66C.

FIG. 67 shows antibody titers of certain FNI3, FNI9, FNI17, or FNI19 mAbs, including gain/loss for variants as compared to wild-type.

FIG. 68 shows binding to group I IAV, group II IAV, and IBV NAs as measured by flow cytometry (reported as MFI) for FNI9 and certain FNI9 variants (FNI9-variant (VH SEQ ID NO:2, VL SEQ ID NO:37) to FNI9-v9). MFI values for variants were normalized to MFI values for wild-type FNI9. The FNI9 variants shown in FIG. 68 are not the same as the FNI9 variant sequences “-VH.6”, “-VH.7”, “-VH.8”, and “-VH.9” as shown in e.g. FIGS. 70-72.

FIGS. 69A-69D show binding kinetics of FNI3-LS, FNI9-LS, FNI17-LS, and FNI19-LS, along with FNI3-LS, FNI9-LS, FNI17-LS, and FNI19-LS antibodies bearing GAALIE mutations (suffix “-GAALIE” in the figure) to different FcγRs, as measured by Bio-Layer Interferometry (BLI). Arrows indicate curves for FNI17-LS and FNI17-LS-GAALIE. FIG. 69A shows binding to FcγRIIA(H), FIG. 69B shows binding to FcγRIIA(R), FIG. 69C shows binding to FcγRIIIA(F), and FIG. 69D shows binding to FcγRIIIA(V).

FIG. 70 shows mutations that were introduced into wild-type FNI9 VH or VL to generate additional variant antibodies.

FIG. 71 shows FNI9 wild-type (“WT”) variable domain amino acid sequences and FNI9 antibody variable domains comprising one or more mutations as shown in FIG. 70.

FIG. 72 summarizes certain FNI9 variant antibodies of the present disclosure (FNI9-v13.8-FNI9-v12.7), comprising one or more mutations as shown in FIG. 70.

FIG. 73 shows FcγR and C1q binding affinity (measured by a meso scale discovery-based assay (MSD; employing electrochemiluminescence)) and other characteristics of certain IgG1 Fc variant antibodies. Fc variants shown beginning in the third row down (“G236A_E272Y_S298N” and below) were identified using an iterative discovery workflow. The G236A_A330L_I332E variant was used as a comparator. Binding of Fc variant antibodies to FcγRIIA-H (high affinity H158 allele), FcγRIIB, FcγRIIA-R (low affinity R131 allele), FcγRIIIA-V (high affinity V158 allele), FcγRIIIA-F (low affinity F158 allele), FcγRIIIB and FcRn was tested. Data are reported as fold-change in binding compared to wild-type IgG1. The ratio of FcγRIIA/FcγRIIB binding, as well as production titer (mg/mL) and Tm (° C.) relative to wild-type IgG1, are also shown.

FIGS. 74A-74C show effect of fucosylation on production and purification of twenty Fc variant antibodies. Variants were expressed in the absence (“No 2FF”) or presence (“+2FF”) of 2-deoxy-2-fluoro-L-fucose (2FF); 2FF reduces fucosylation. FIG. 74A shows antibody titers as determined using a Protein A column. FIG. 74B shows yields resulting from two replicate purifications. The table in FIG. 74C summarizes the theoretical maximum yield and average yield, both measured in μg, along with the calculated average recovery and protein concentration of the second elution (measured in μg/ml). Fc variants were purified using two elutions and combined prior to determining yield.

FIGS. 75A-75B summarize FcγR binding and other characteristics of Fc variants, relative to wildtype Fc. Bars and values indicate fold-change in binding as compared with wild-type Fc. Fc variants shown were not treated with 2FF. FIG. 75A shows binding to FcγRIIA-H (high affinity), FcγRIIA-R (low affinity), FcγRIIB, FcγRIIIA-V (high affinity), FcγRIIIA-F (low affinity), and FcRn (at pH 6). FIG. 75B further shows the ratio of FcγRIIA-H/FcγRIIB binding, as well as C1q binding and complement-dependent cytotoxicity (CDC), with the WT “baseline” value indicated by a dashed vertical red line. Binding was measured by a meso scale discovery-based assay (MSD; employing electrochemiluminescence)).

FIG. 76 shows binding of certain Fc variants to FcγRIIA-H (high affinity) and FcγRIIB. Plots connected by a line represent the same variant. Variants shown were not treated with 2FF.

FIGS. 77A-77B show FcγR signaling through different FcγRs as measured using a reporter cell assay (Promega; tested cells expressed one type/allele FcγR, as indicated). Fc variants shown are fucosylated (“fuc”; 124A/124B) or afucosylated (“afuc”; 124B) as indicated in the figure. Values are calculated from an average of three experiments and indicate fold-change (expressed linear) in area-under-the-curve (plotted in log) as compared to wildtype Fc.

FIG. 78 summarizes characteristics of certain variant Fcs. Antibodies comprising the indicated Fc were expressed as recombinant human IgG1. Binding was measured by a meso scale discovery-based assay (MSD; employing electrochemiluminescence)). Values represent fold-change compared to the antibody comprising wild-type fucosylated human IgG1 Fc. Also shown is fold-change in FcγR signaling, as measured using a reporter cell assay.

FIG. 79 shows results from experiments measuring: FcγR-binding; ratio of binding of FcγRIIA alleles to FcγRIIB; C1q-binding; melting temperature; and FcRn-binding, by certain Fc variant antibodies. Anti-influenza A antibody FY1 (Kallewaard et al., Cell. 2016 Jul. 28; 166(3):596-608) was expressed as recombinant IgG1m3 with M428L and N434S mutations in CH3, and with the indicated combination mutations elsewhere in the Fc. Binding (one study) was measured by a meso scale discovery-based assay (MSD; employing electrochemiluminescence)). Binding data are shown as fold-change relative to FY1 rIgG1m3-MLNS without the other Fc mutations. FcγR-binding was confirmed by FcγR-signaling using a reporter cell assay (NFAT driving luciferase) (Promega).

FIG. 80 shows results from additional experiments measuring the antibody features as in FIG. 79. In these experiments, FY1 was expressed as recombinant IgG1m3 without the M428L and N434S mutations (i.e., with wild-type IgG1m3 CH1-CH3 or with the mutations indicated in the table). The FYT-rIgG1m3 and FYT-rIgG1m3-GAALIE antibodies were produced and measured twice independently in a first plate; averaged data is shown. FY1-rIgG1m3-GA antibody was produced 2× independently in a first and a second plate. For the other variants, a single measurement was performed. Binding was measured by a meso scale discovery-based assay (MSD; employing electrochemiluminescence)). Binding data are shown as fold-change relative to FY1 rIgG1m3 with wild-type Fc. FcγR-binding/activation was using a reporter cell assay (NFAT driving luciferase) (Promega).

FIG. 81 shows results from additional experiments measuring features as in FIG. 80, using afucosylated Fc variant antibodies. Antibodies were produced in the presence of 2FF to obtain afucosylated glycans. In these experiments, FY1 was expressed as recombinant IgG1m3 without the M428L and N434S mutations (i.e., with wild-type IgG1m3 CH1-CH3 or with the mutations indicated in the table). The FY1-rIgG1m3 and FY1-rIgG1m3-GAALIE antibodies were produced and measured twice independently in a first plate; averaged data is shown. FY1-rIgG1m3-GA antibody was produced 2× independently in a first and a second plate. For the other variants, a single measurement was performed. Binding was measured by a meso scale discovery-based assay (MSD; employing electrochemiluminescence)).

FIG. 82 shows FcγRIIA activation/signaling by anti-influenza FY1 antibodies with variant Fc, as indicated in the key. Target cells were A549 cells expressing FluA H1N1 HA, and reporter cells were Jurkat cells expressing FcγRIIA (H131 allele) and luciferase under control of a NFAT promoter.

FIGS. 83A-83B show FcγRIIIA activation/signaling by anti-influenza FY1 antibodies with variant Fc, as indicated in the key. Target cells were A549 cells expressing FluA H1N1 HA, and reporter cells were Jurkat cells expressing FcγRIIIA (F158 lower-affinity allele (A) or V158 higher-affinity allele (B)) and luciferase under control of a NFAT promoter.

FIGS. 84A-84I relate to certain anti-HBV (“HBC34-v40”; see PCT publication no. WO 2021/262840) Fc variant antibodies. (A) Flow cytometry showing CD83 expression on monocyte-derived dendritic cells (moDCs) (expressing the indicated FcγR) in the presence of the indicated HBC34-v40 Fc variant antibody (50 μg/mL) and 30 IU/mL HBsAg from HBV+ patient serum. (B) Flow cytometry showing CD83 expression on moDCs in the presence of HBC34-v40 Fc variant antibody (50 μg/mL) and HBsAg from HBV+ patient serum (BioIVT) at the indicated concentration. Left graphs are from an experiment using a first method of pipetting/generating immune complexes of antibody:HBsAg; right graphs are from an experiment using a second method of pipetting/generating immune complexes of antibody:HBsAg. (C) CD25 expression (marker of activation) and CFSE (proliferation) on autologous CD4+ memory T cells (from an HBV vaccinee) incubated for 5 days with moDCs from the same donor; moDCs were first activated overnight with 100 IU/mL HBsAg (from two patient sera) and 50 μg/mL HBC34-v40 Fc variant antibody. The LS-GAYL variant was compared in one experiment. (D) CD14+ monocytes were stimulated with IL-4 and GM-CSF for 6 days. MoDCs were treated with antigen and HBC34-v40 Fc variant antibody (50 μg/mL) overnight, then co-cultured with an HLA-matched (HLA-DR-restricted) transgenic Jurkat cells expressing an HBsAg-specific human TCR. The readout was GFP-NFAT reporter of Jurkat cells. (E) Comparison of Jurkat TCR reporter assay for three independent experimental repeats at 0.125 μg/mL antibody. (F) Summary of data from different assays. (G) Scheme showing experimental setup for assessing ex vivo proliferation of T cells from FcγR-expressing mice immunized and boosted with an HBsAg vaccine; memory CD44+CD4+ T cells were sorted, labeled with CFSE, co-cultured with immune complex (antibody:HBsAg antigen)-pulsed BMDCs, and assessed for proliferation on day 6. SEB=Staphylococcal enterotoxin B from S. aureus. (H) CD4 expression and CFSE staining on (500,000) CD4+ memory T cells as in (G), wherein the BMDCs (50,000) were stimulated using immune complexes comprising the indicated HBC34-v40 Fc variant antibody (20 μg/mL) and HBsAg (1000 IU/mL). SEB=1 μg/mL; Mann-Whitney test. (I) (Left) Frequency of CFSE low CD4+CD44+ T cells following incubation with moDCs pre-treated with HBsAg alone, antibody alone, or SEB; (Right) Frequency of CFSE low CD4+CD44+ T cells following incubation with moDCs pre-treated with HBsAg and the indicated HBC34-v40 Fc variant antibody at the indicated concentration. moDCs were from mice transgenically expressing human FcγRs, and T cells were from either HuFcγR mice (n=4 independent experiments) or C57Bl/6 mice (n=1 experiment). 50,000 moDCs+500,000 T cells were tested. SEB=1 μg/mL; Mann-Whitney test.

FIGS. 85A-85B show in vitro inhibition of sialidase activity by FNI9 and FNI9-v5 against NAs as measured by MUNANA assay. FIG. 85A shows inhibition of sialidase activity against N1 from H5N1 A/Vietnam/1203/2004. FIG. 85B shows inhibition of sialidase activity against N2 from H3N2 A/Tanzania/205/2010.

FIG. 86 shows binding affinity of FNI9 and FNI9-v5 Fab fragments to N2 antigens with or without a glycosylation site at position 245, as measured by surface plasmon resonance (SPR). Binding affinity is reported as the equilibrium constant, KD, in nM. The label “+glcy245” indicates a glycosylation site is present in position 245. Asterisks indicate the method by which the Fab fragment was generated, where “r” indicates generation by recombinant expression and “d” indicates generation by digestion.

FIGS. 87A-87B show in vitro inhibition of N2 sialidase activity in pseudotype virus particles by FNI9 and FNI9-v5 as measured by ELLA (enzyme-linked lectin assay). FIG. 87A shows inhibition of sialidase activity against A/Switzerland/2017 pseudovirus particles. FIG. 87B shows inhibition of sialidase activity against A/Kansas/14/2017 pseudovirus particles.

FIG. 88 shows in vitro neutralization of influenza (reported as EC50 in μg/ml) by FNI9 and FNI9-v5.

FIG. 89 summarizes mutations in the VH and VL (VK) domains of certain variants of the FNI9 parental antibody. The “FNI9” and “FNI9-v1.1” antibodies have the amino acid sequences. FNI9-v1.1 was produced concurrently with the variants to control for any variance in production conditions.

FIG. 90 shows production titer and size exclusion chromatography (SEC) profiles for FNI9 antibodies. An SEC profile of “ok” indicates no significant aggregation (<3% high molecular weight species) or fragmentation (<3% low molecular species) observed by UHPLC-SEC. An SEC profile of “Not Assessed” indicates that the SEC profile could not be assessed as the antibody was not produced at sufficient titer in transient transfection. For productivity, “too low” indicates the antibody titer in supernatants of transiently transfected cells was below the detection limit of the quantification method (protein A binding on BLI). “FNI9-v1.1” has the same sequences as FNI9.

FIGS. 91A-91B show in vitro inhibition of sialidase activity by FNI9 variants against N1, N2, and N9 NAs as measured by MUNANA assay. Values indicate antibody concentration (in μg/ml) resulting in 50% inhibition of sialidase activity. Neuraminidase antigen concentrations used in the assay are indicated in the key. “FNI9-v1.1” has the same sequences as FNI9.

FIGS. 92A-92B show in vitro inhibition of sialidase activity by FNI9 variants against the indicated pseudovirus-derived NAs as measured by ELLA. Values indicate antibody concentration (in μg/ml) resulting in 50% inhibition of sialidase activity. Neuraminidase antigen concentrations used in the assay are indicated in the key. “FNI9-v1.1” has the same sequences as FNI9.

FIGS. 93A-93D show binding of FNI9 antibodies to NAs transiently expressed in mammalian cells as measured by flow cytometry and reported in MFI. FIG. 93A shows binding to N1 from A/California/07/2009 and A/California/07/2009 I223R/H275Y, and N2 in A/Washington/01/2007 and A/Washington/01/2007 R292K. FIG. 93B shows binding to N2 from A/Switzerland/8060/2017, A/Kansas/14/2017, A/Cambodia/2020, and A/South Australia/34/2019. FIG. 93C shows binding to IBV NAs from B/Malaysia/2506/2004 (Victoria), B/Brisbane/2008 (Victoria), B/Yamanashi/166/1998 (Yamagata), and B/Phuket/3073/2013 (Yamagata). FIG. 93D shows binding to N2 from A/Leningrad/134/17/57 and A/Perth/16/2009, and N9 from A/Anhui/1/2013. In FIGS. 93A-93D, the bars in each graph, from left to right for each test condition (e.g., for “N1_A_Calif_07_2009” in FIG. 93A), correspond to the antibodies as indicated top-to-bottom and left to right in the respective figure key. For example, the first three bars from the left in FIG. 93A above “N1_A_Calif_07_2009” correspond to FNI9v-1.1, FNI9-v4.1, and FNI9-v4.7. Antibodies FNI9-v1.1, FNI9-v4.1, FNI9-v8.1, FNI9-v13.8, and FNI9-v9.1 are indicated by patterns or shading in the bars.

FIG. 94 shows affinity of antibodies “FNI9-v4.1”, “FNI9-v8.1”, “FNI9-v9.1”, and “FNI9-v13.8” (expressed as IgG1) for IAV and IBV NA antigens, as measured by SPR. NA antigen categories are indicated, wherein “FluB” indicates NAs from IBVs, “N1” indicates N1 NAs, “A/H3N2 −gly245” indicates N2 NAs from H3N2 strains without glycosylation at position 245, “A/H3N2 +gly245” indicates N2 NAs from H3N2 strains with glycosylation at position 245, and “N9” indicates a N9 NA. Values indicate fold-change in affinity as compared to the parental antibody “FNI9-v1.1” (same sequences as FNI9).

FIGS. 95A-95B show affinity of Fab fragments from FNI9-v1.1 (same sequences as FNI9), FNI9-v4.1, FNI9-v8.1, and FNI9-v13.8 for N2 antigens, as measured by SPR. Binding to A/Tanzania/205/2010, A/South Australia/34/2019, and A/HongKong/2671/2019 with (labelled “+gly245”) or without (labelled “−gly245”) glycosylation at position 245 was measured. FIG. 95A shows binding affinity reported as the equilibrium constant, KD, in nM. FIG. 95B shows binding affinity reported as fold-change compared to Fab from the parental antibody, FNI9-v1.1.

FIGS. 96A-96C show binding kinetics of FNI9-v1.1 (same sequences as FNI9) (FIG. 96A), FNI9-v8.1 (FIG. 96B), and FNI9-v9.1 (FIG. 96C) to N9 NAs, as measured by Bio-Layer Interferometry (BLI). Dissociation is reported as kdis (1/s), association is reported as ka (1/Ms), and KD (M) was calculated from the ratio of kdis/ka.

FIG. 97A shows in vitro neutralizing activity of FNI9-v1.1 (same sequences as FNI9), FNI9-v8.1, FNI9-v4.1, FNI9-v9.1, and FNI9-v13.8 (reported as EC50 in μg/ml) against a panel of seasonal IAVs and IBVs. Strains bearing a glycosylation at position 245 are as indicated. FIG. 97B shows in vitro inhibition of sialidase activity (reported as IC50 in ng/ml) by FNI9-v1.1 and FNI9-v8.1 against a panel of seasonal group I (H1N1) IAV, group II (H3N2) IAV, and IBV NAs.

FIGS. 98A-98G show in vitro inhibition of sialidase activity by certain FNI9 variants against N1, N2, and N9 NAs as measured by MUNANA assay. FNI9-v4.1, FNI9-v4.7, FNI9-v5.1, FNI9-v5.7, FNI9-v6.1, FNI9-v6.7, FNI9-v7.1, FNI9-v7.7, FNI9-v8.7, FNI9-v8.1, and FNI9-v13.8 antibodies were tested. FNI9, FNI9-v1.1, and FNI9-v5 were tested as comparator antibodies. Inhibition of sialidase activity against N1 in A/Vietnam/1203/2004 (FIG. 98A), N2 in A/Tanzania/205/2010 (FIG. 98B), N2 in A/Switzerland/8060/2017 (FIG. 98C), N2 in A/South Australia/34/2019 (FIG. 98D), N2 in A/HongKong/2671/2019 (FIG. 98E), N2 in A/Tanzania/205/2010 (+Gly245) (FIG. 98F), and N9 in A/Hong Kong/56/2015 (FIG. 98G) is shown.

FIGS. 99A-99G show in vitro inhibition of sialidase activity by certain FNI9 variants against N1, N2, and N9 NAs as measured by MUNANA assay. FNI9-v6.1, FNI9-v9.1, FNI9-v9.7, FNI9-v11.1, FNI9-v11.7, FNI9-v12.1, and FNI9-v12.7 antibodies were tested. FNI9, FNI9-v1.1 (same as FNI9, produced with the variants to control for any variance in production conditions), and FNI9-v5 were tested as comparator antibodies. Inhibition of sialidase activity against N1 in A/Vietnam/1203/2004 (FIG. 99A), N2 in A/Tanzania/205/2010 (FIG. 99B), N2 in A/Switzerland/8060/2017 (FIG. 99C), N2 in A/South Australia/34/2019 (FIG. 99D), N2 in A/HongKong/2671/2019 (FIG. 99E), N2 in A/Tanzania/205/2010 (+Gly245) (FIG. 99F), and N9 in A/Hong Kong/56/2015 (FIG. 99G) is shown.

FIGS. 100A-100F show in vitro inhibition of sialidase activity by certain FNI9 variants against the indicated pseudovirus-derived NAs as measured by ELLA. FNI9-v4.1, FNI9-v4.7, FNI9-v5.1, FNI9-v5.7, FNI9-v6.1, FNI9-v6.7, FNI9-v7.1, FNI9-v7.7, FNI9-v8.7, FNI9-v8.1, and FNI9-v13.8 antibodies were tested. FNI9, FNI9-v1.1, and FNI9-v5 were tested as comparator antibodies. Inhibition of sialidase activity against H7N3 A/Ck/Ja/2017 (FIG. 100A), H5N6 A/Ck/Suzhou/2019 (FIG. 100B), H5N6 A/Hangzhou/2021 (FIG. 100C), H7N7 A/Ck/621572/03 (FIG. 100D), H5N8 A/Ck/Russia/2020 (FIG. 100E), and H7N9 A/Anhui/i/2013 (FIG. 100F) is shown.

FIGS. 101A-101F show in vitro inhibition of sialidase activity by certain FNI9 variants against the indicated pseudovirus-derived NAs as measured by ELLA. FNI9-v6.1, FNI9-v9.1, FNI9-v9.7, FNI9-v11.1, FNI9-v11.7, FNI9-v12.1, and FNI9-v12.7 antibodies were tested. FNI9, FNI9-v1.1, and FNI9-v5 were tested as comparator antibodies. Inhibition of sialidase activity against H7N3 A/Ck/Ja/2017 (FIG. 101A) H5N6 A/Ck/Suzhou/2019 (FIG. 101B) H5N6 A/Hangzhou/2021 (FIG. 101C) H7N7 A/Ck/621572/03 (FIG. 101D) H5N8 A/Ck/Russia/2020 (FIG. 101E), and H7N9 A/Anhui/1/2013 (FIG. 101F) is shown.

FIGS. 102A-102B show in vitro neutralizing activity measured by nucleoprotein (NP) staining of FNI9, FNI19-v3, FNI17-v19, and FNI17-v19-LS, against group I (H1N1) IAV, group II (H3N2) IAV, and IBV NAs. The rectangle indicates group II (H3N2) NAs harboring glycosylation at position 245. Neutralizing activity of comparator antibody, 1G01, was also measured.

FIGS. 103A-103B show an estimation of median EC90 values determined by non-linear regression (reported in μg/ml) for FNI9-v1.1 or FNI9-v8.1 against a panel of group I (H1N1) IAV, group II (H3N2) IAV, and IBV NAs.

FIG. 104 summarizes median EC90, tissue-adjusted EC90 at 25%, and tissue-adjusted EC90 at 5% data by FNI9-v8.1 against a panel of group I (H1N1) IAV, group II (H3N2) IAV (with or without Glycan-245), and IBV NAs.

FIG. 105 shows in vitro neutralizing activity of FNI9 (reported in μg/ml) against H3N2 A/Singapore/INFIMH-16-0019/2016. Neutralizing activity of an anti-HA comparator antibody, FM08-LS, was also measured.

FIG. 106 shows a dose response curve of in vitro neutralizing activity by FNI9 and FNI17-v19 against H3N2 A/Singapore/INFIMH-16-0019/2016.

FIG. 107 shows viral titer in lung homogenates from BALB/c mice treated with FNI9-v1.1, FNI9-v8.1, or FNI17-v19 prior to infection with H3N2 A/Singapore/INFIMH-16-0019/2016. Antibody was administered at 3 mg/kg, 0.9 mg/kg, 0.3 mg/kg, 0.1 mg/kg, or 0.03 mg/kg. Viral titers were also measured in mice treated with OSE (10 mg/kg or 20 mg/kg) or an anti-HA comparator antibody, FM08_LS. Lung tissue was collected at four days post-infection. Titer is reported as log 50% tissue culture infectious dose per gram tissue (Log TCID50/g).

FIGS. 108A-108D show viral titer in lung homogenates from BALB/c mice treated with FNI9-v8.1 prior to infection with an IAV or IBV. Antibody was administered at 3 mg/kg, 0.9 mg/kg, 0.3 mg/kg, or 0.1 mg/kg prior to infection with H1N1 A/Puerto Rico/8/34 (FIG. 108A), H3N2 A/Singapore/INFIMH-16-0019/2016 (FIG. 108B), B/Victoria/504/2000 (Yamagata) (FIG. 108C), or B/Brisbane/60/2008 (Victoria) (FIG. 108D). Viral titers were also measured in mice treated with OSE (10 mg/kg or 20 mg/kg) or an anti-HA comparator antibody, FM08_LS. Lung tissue was collected at four days post-infection. Titer is reported as log 50% tissue culture infectious dose per gram tissue (Log TCID50/g).

FIGS. 109A-109D show viral titer in lung homogenates from BALB/c mice treated with FNI9-v8.1 prior to infection with an IAV or IBV. Antibody was administered at 3 mg/kg, 0.9 mg/kg, 0.3 mg/kg, or 0.1 mg/kg prior to infection with H1N1 A/Puerto Rico/8/34 (FIG. 109A), H3N2 A/Singapore/INFIMH-16-0019/2016 (FIG. 109B), B/Victoria/504/2000 (Yamagata) (FIG. 109C), or B/Brisbane/60/2008 (Victoria) (FIG. 109D). Titer is reported as log plaque-forming units per gram tissue (Log pfu/g).

FIGS. 110A and 110B show in vitro neutralization data by FNI9-v8.1 against a panel of H1N1 IAVs, H3N2 IAVs (with or without a glycosylation site at position 245), and IBVs.

FIG. 111 summarizes in vitro neutralization statistics by FNI9-v8.1 against all strains, H1N1 IAVs, H3N2 IAVs, H3N2 IAVs (without a glycosylation site at position 245), H3N2 IAVs (with a glycosylation site at position 245), and IBVs.

FIGS. 112A-112B show in vitro neutralizing activity of FNI9-v8.1, FNI17, and FNI19 against group I (H1N1) IAV, group II (H3N2) IAV, and IBV NAs, as measured by nucleoprotein (NP) staining. Neutralizing activity of comparator antibody, 1G01, was also measured. For each strain, data for two independent experiments is shown.

FIGS. 113A-113D show in vitro neutralization matrixes (FIGS. 113A and 113C) and synergy plots (FIGS. 113B and 113D) reporting combination activity of FM08 and FNI9-v8.1 against H1N1 A/Puerto Rico/34 (FIGS. 113A and 113B) and H3N2 A/Tasmania/503/2020 (FIGS. 113C and 113D).

FIGS. 114A-114B show complement-dependent cytotoxicity (CDC) mediated by FNI9-v8.1-LS (comprising M428L and N434S mutations in the Fc) on MDCK-LN cells infected with H1N1 A/Puerto Rico/8/34 in the presence of guinea pig complement. CDC of an anti-HA comparator antibody, FM08_LS, and a Fc-silent negative control, FNI9-v8.1-GRLR, was also measured. CDC is reported as % antibody-dependent killing in FIG. 114A and area-under-the-curve in FIG. 114B.

FIGS. 115A-115B show antibody-dependent cell cytotoxicity (ADCC) mediated by FNI9-v8.1-LS on A549 cells infected with H1N1 A/Puerto Rico/8/34 in the presence of human natural killer cells. ADCC of an anti-HA comparator antibody, FM08_LS, and a Fc-silent negative control, FNI9-v8.1-GRLR, was also measured. ADCC is reported as % antibody-dependent killing in FIG. 115A and area-under-the-curve in FIG. 115B.

FIGS. 116A-116B show antibody-dependent cellular phagocytosis (ADCP) with serial dilution of FNI9-v8.1-LS using peripheral blood mononuclear cells (PBMCs) as source of monocyte and PKH67-labelled ExpiCHO cells expressing N2 NA as target cells. The y axis indicates the percentage of monocytes double positive for CD14 and PKH67. For all assays FNI9-v8.1-GRLR is used as Fc-silent negative control and results are showed both as dose-response curves (a, b, c) and as area under the curve (AUC) (d, e, f). representative of n=1 (d, e) or n=2 (f) experimental replicates (black dots).

FIGS. 117A-117D show correlation between antibody concentration at Day 0 and virus lung titers in vivo related to FIGS. 108A-108D. Infectious virus titres in the lungs at 4 days after infection plotted as a function of serum mAb concentrations before infection (day 0) with H1N1 A/Puerto Rico/8/34, H3N2 A/Singapore/2016, B/Victoria/504/2000, and B/Brisbane/60/2008 viruses.

FIGS. 118A-118F show inhibition of enzymatic activity as measured by ELLA exerted by anti-NA mAbs on NAs not bearing (−) or bearing (+) glycan at position 245 transiently expressed in mammalian cells.

FIGS. 119A-119D show inhibition of enzymatic activity (IC50 values) as measured by ELLA exerted by anti-NA mAbs against NA-only based pseudotypes bearing N3, N6, N7, N8 or N9 representative of highly pathogenic avian influenza A viruses (HPAIVs) that were previously reported to infect humans (Ke et al., 2017; Li et al., 2022; WHO, 2021). Additional data are shown in FIGS. 133A-133C.

FIGS. 120A-120D show inhibition of NA enzymatic activity by the anti-NA antibodies against group 1 (N1) and group 2 (N2) IAV (A, B) and Yamagata and Victoria lineages IBV (C, D) NA antigens, as measured by MUNANA assay.

FIGS. 121A-121H show conservation analysis of FNI antibodies' key contacting residues. Mean conservation percentage from 2000 to 2022 of key NA contacting residues (R118, D151, E227, R292, and R371) per year (red line). The number of sequences analysed per year is indicated (black bar). H1N1, H3N2 and IBVs isolates are of human origin while for H5N1, H7N9, H5N8 and H5N6 viruses from all animal reservoirs are included.

FIGS. 122A-122E show detailed structural analysis of FNI antibodies binding to NA. Provided data: (A) Static epitope analysis of FNI9:NA (Tanzania/2010) is depicted as a heatmap whose shading scale is MOE kcal/mol energy, with density plots compressing all energies by epitope residues and by paratope residues. The total energy was −230 MOE kcal/mol. Static epitope analysis of FNI17:NA (Tanzania/2010) depicted the same. The total energy was −240 MOE kcal/mol. (B) Dynamic epitope-paratope analysis of FNI9:NA (Tanzania/2010), depicted as described in (A) except the energies are the average across 11.5 μs aggregate fully atomistic MD simulations (every 10 ns) using the same MOE energy analysis as described above for (A). The total energy was −370 MOE kcal/mol. (C) Percent occupancy of epitope-paratope interactions reflect the fraction of the 11.5 μs MD where contacts were present (within 5 Å). The total occupancy was 5600(%). The total occupancy of epitope-paratope interactions of FNI17:NA was 4700(%). Outlined boxes depict epitope residue-paratope residue contacts present in the FNI9 dynamic paratope analysis that are absent in the FNI17 dynamic paratope analysis. (D) Comparison of FNI9 and FNI17 epitope interaction energies, where black stars depict contacts present in FNI9 and not FNI17, white stars contacts present in FNI17 and not FNI9. (E) Comparison of FNI9 and FNI17 paratope interaction energies, with black and white stars as in (D).

FIG. 123 shows a list of flu strains and neutralization EC50s for the indicated anti-NA antibodies.

FIG. 124 shows a list of NA antigens from seasonal viruses with or without glycan 245 and corresponding affinity (KD) to certain FNI antibody Fabs and 1G01 Fab by SPR.

FIGS. 125A-125B show in vivo pharmacokinetics of FNI9-v5 (FIG. 125A) and FNI9-v8.1 (FIG. 125B) as concentration over time (reported as μg/ml) in SCID tg32 mice over approximately 60 days post-administration. Antibodies were expressed as recombinant IgG1m3 with M428L and N434S (“LS”, also referred to as “MLNS”) mutations in the Fc.

FIGS. 126A-126E summarize in vivo pharmacokinetic data for FNI9-v5-LS (“FNI9-v5-rIgG1-LS”) and FNI9-v8.1-LS (“FNI9-v8.1-rIgG1m3-LS”) in SCID tg32 mice. Data for five individual animals is shown.

FIGS. 127A-127C summarize results of tissue cross-reactivity (TCR) studies using FNI9-v8.1 (conjugated to Alexa Fluor 488) against a panel of human tissues as measured using immunohistochemistry staining.

FIGS. 128A-128C show inhibition of neuraminidase enzymatic activity as measured by ELLA exerted by anti-NA mAbs FNI9-v8.1, FNI17-v19, and FNI19-v3 against NA-only based pseudotypes bearing N3, N4, or N5 representative of enzootic low pathogenic avian influenza A viruses (LPAIVs). 1G01 was also tested as a control antibody.

FIGS. 129A-129F show inhibition of neuraminidase enzymatic activity as measured by MUNANA assay exerted by anti-NA mAbs FNI9-v8.1, FNI17-v19, and FNI19-v3 against NA-only based pseudotypes bearing N1, N2, N6, or N8 representative of enzootic low pathogenic mammalian IAVs circulating in swine, dogs, and seals. 1G01 was also tested as a control antibody.

FIGS. 130A-130G show inhibition of neuraminidase enzymatic activity as measured by MUNANA assay against a panel of pseudotypes bearing NAs with mutations identified through in vitro resistance studies as capable of reducing, but not abrogating, the binding or activity of FNI mAbs.

FIGS. 131A-131B show inhibition of neuraminidase enzymatic activity as measured by MUNANA assay against a panel of pseudotypes bearing NAs with mutations identified through Deep Mutational Scanning (DMS) as capable of reducing, but not abrogating, the binding or activity of FNI mAbs.

FIGS. 132A-132C show body weight loss from day 0 to 14 post-infection (reported as area-under-the-curve) in BALB/c mice (n=6) infected with H3N2 A/Hong Kong/1/68 (FIGS. 132A and 132C) or H1N1 A/Puerto Rico/8/34 (FIG. 132B) following pre-treatment with an anti-NA antibody, an anti-HA antibody, or one of both an anti-NA and an anti-HA antibody at a 1:1 ratio. Antibodies were murinized (indicated by a “mu” prefix in FIG. 132A) or human (FIGS. 132B and 132C). Anti-NA antibodies included FNI9-v8.1-LS, FNI17-LS, and FNI19-v3-LS, and the anti-HA antibody used was FM08. Antibody was administered at a dose of 0.25 mg/kg or 0.125 mg/kg. Body weight loss in mice pre-treated with a vehicle control was also measured.

### DETAILED DESCRIPTION

Provided herein are antibodies and antigen-binding fragments that can bind to and potently neutralize infection by various influenza viruses, such as influenza A viruses (IAVs) and influenza B viruses (IBVs). Also provided are polynucleotides that encode the antibodies and antigen-binding fragments, vectors, host cells, and related compositions, as well as methods of using the antibodies, nucleic acids, vectors, host cells, and related compositions to treat (e.g., reduce, delay, eliminate, or prevent) an influenza virus infection in a subject and/or in the manufacture of a medicament for treating an influenza infection in a subject.

As taught in the present examples, a number of clonally related antibodies were identified that bind to a breadth of IAV and IBV NAs and have neutralizing/inhibitory functions against IAV and IBV viruses. Certain antibodies, including “FNI9”, have improved functions as compared to the antibody “IG01” (described by Stadlbauer et al. (Science 366(6464):499-504 (2019)). Disclosed antibodies and antigen-binding fragments include variants engineered from antibody FNI9. In some embodiments, an antibody or antigen-binding fragment has at least substantially equivalent, equivalent, or improved: breadth of binding; in vitro production titers; neuraminidase inhibition; and/or neutralization potency as compared to FNI9 (or an antigen-binding fragment thereof). In some embodiments, an antibody or antigen-binding fragment of the present disclosure has a size exclusion chromatography (SEC) profile with no significant aggregation (<3% high molecular weight species) or fragmentation (<3% low molecular species) observed by UHPLC-SEC. Certain variant antibodies disclosed herein (e.g., “FNI9-v8.1”) have one or more improved function as compared to the parental antibody FNI9.

Sequence variants of the antibodies were generated and characterized. Certain disclosed embodiments relate to such antibodies, antigen-binding fragments of the same, and related compositions and uses.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain) or a protein “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid, and non-naturally occurring amino acid polymers. Variants of proteins, peptides, and polypeptides of this disclosure are also contemplated. In certain embodiments, variant proteins, peptides, and polypeptides comprise or consist of an amino acid sequence that is at least 70%, 75%, 80%, 85%, 90%, 91%, 92%, 93%, 94%, 95%, 96%, 97%, 98%, 99%, or 99.9% identical to an amino acid sequence of a defined or reference amino acid sequence as described herein.

Any polypeptide of this disclosure (e.g., VH, VL, antibody heavy chain, antibody light chain) can, as encoded by a polynucleotide sequence, comprise a “signal peptide” (also known as a leader sequence, leader peptide, or transit peptide). Signal peptides target newly synthesized polypeptides to their appropriate location inside or outside the cell. A signal peptide may be removed in whole or in part from the polypeptide during or once localization or secretion is completed. Polypeptides that have a signal peptide can be referred to as a “pre-protein” and polypeptides having their signal peptide removed can be referred to as “mature” proteins or polypeptides. In certain embodiments, an antibody or antigen-binding fragment is a mature protein or a pre-protein.

“Nucleic acid molecule” or “polynucleotide” or “polynucleic acid” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), which includes mRNA, microRNA, siRNA, viral genomic RNA, and synthetic RNA, and polydeoxyribonucleic acid (DNA, also referred to as deoxyribonucleic acid), which includes cDNA, genomic DNA, and synthetic DNA, either of which may be single or double stranded. If single-stranded, the nucleic acid molecule may be the coding strand or non-coding (anti-sense) strand. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

In some embodiments, the polynucleotide comprises a modified nucleoside, a cap-1 structure, a cap-2 structure, or any combination thereof. In certain embodiments, the polynucleotide comprises a pseudouridine, a N6-methyladenonsine, a 5-methylcytidine, a 2-thiouridine, or any combination thereof. In some embodiments, the pseudouridine comprises N1-methylpseudouridine. These features are known in the art and are discussed in, for example, Zhang et al. Front. Immunol., DOI=10.3389/fimmu.2019.00594 (2019); Eyler et al. PNAS 116(46): 23068-23071; DOI: 10.1073/pnas.1821754116 (2019); Nance and Meier, ACS Cent. Sci. 2021, 7, 5, 748-756; doi.org/10.1021/acscentsci.1c00197 (2021), and van Hoecke and Roose, J Translational Med 17:54 (2019); https://doi.org/10.1186/s12967-019-1804-8, which modified nucleosides and mRNA features are incorporated herein by reference. Variants of nucleic acid molecules of this disclosure are also contemplated. Variant nucleic acid molecules are at least: 70%, 75%, 80%, 85%, 90%, and are preferably 95%, 96%, 97%, 98%, 99%, or 99.9% identical (i.e., at least 70%, at least 75%, at least 80%, or at least 90%, and preferably at least 95%, 96%, 97%, 98%, 99%, or 99.9% identical) to a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a binding domain thereof having a functionality described herein, such as binding a target molecule.

“Percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Other examples include Clustal W, MAFFT, Clustal Omega, AlignMe, Praline, GAP, BESTFIT, Needle (EMBOSS), Stretcher (EMBOSS), GGEARCH2SEQ, Water (EMBOSS), Matcher (EMBOSS), LALIGN, and SSEARCH2SEQ. A global alignment algorithm, such as a Needleman and Wunsch algorithm, can be used to align two sequences over their entire length, maximizing the number of matches and minimizes the number of gaps. Default values can be used.

To generate similarity scores for two amino acid sequences, scoring matrices can be used that assign positive scores for some non-identical amino acids (e.g., conservative amino acid substitutions, amino acids with similar physio-chemical properties, and/or amino acids that exhibit frequent substitutions in orthologs, homologs, or paralogs). Non-limiting examples of scoring matrices include PAM30, PAM70, PAM250, BLOSUM45, BLOSUM50, BLOUM62, BLOSUM80, and BLOSUM90.

Within the context of this disclosure, it will be understood that where sequence analysis software is used for analysis, the results of the analysis are based on the “default values” of the program referenced. “Default values” mean any set of values or parameters which originally load with the software when first initialized.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. “Isolated” can, in some embodiments, also describe an antibody, antigen-binding fragment, polynucleotide, vector, host cell, or composition that is outside of a human body. In certain embodiments, an isolated antibody, antigen-binding fragment, polynucleotide, vector, host cell, or composition is provided.

The term “gene” means the segment of DNA or RNA involved in producing a polypeptide chain; in certain contexts, it includes regions preceding and following the coding region (e.g., 5′ untranslated region (UTR) and 3′ UTR) as well as intervening sequences (introns) between individual coding segments (exons).

A “functional variant” refers to a polypeptide or polynucleotide that is structurally similar or substantially structurally similar to a parent or reference compound of this disclosure, but differs slightly in composition (e.g., one base, atom or functional group is different, added, or removed), such that the polypeptide or encoded polypeptide is capable of performing at least one function of the parent polypeptide with at least 50% efficiency, preferably at least 55%, 60%, 70%, 75%, 80%, 85%, 90%, 95%, 96%, 97%, 98%, 99%, 99.9%, or 100% level of activity of the parent polypeptide. In some embodiments, an encoded polypeptide or polypeptide is capable of performing at least one function of the parent polypeptide with at least 55%, at least 60%, at least 70%, at least 75%, at least 80%, at least 85%, at least 90%, at least 95%, at least 96%, at least 97%, at least 98%, at least 99%, at least 99.9%, or at least 100% level of activity of the parent polypeptide. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Ka) or a dissociation (KD) constant).

As used herein, a “functional portion” or “functional fragment” refers to a polypeptide or polynucleotide that comprises only a domain, portion or fragment of a parent or reference compound, and the polypeptide or encoded polypeptide retains at least 50% activity associated with the domain, portion or fragment of the parent or reference compound, preferably at least 55%, 60%, 70%, 75%, 80%, 85%, 90%, 95%, 96%, 97%, 98%, 99%, or 99.9%, or 100% level of activity of the parent polypeptide, or provides a biological benefit (e.g., effector function). In some embodiments, a polypeptide or encoded polypeptide retains at least 55%, at least 60%, at least 70%, at least 75%, at least 80%, at least 85%, at least 90%, at least 95%, at least 96%, at least 97%, at least 98%, at least 99%, or at least 99.9%, or at least 100% level of activity of the parent polypeptide. A “functional portion” or “functional fragment” of a polypeptide or encoded polypeptide of this disclosure has “similar binding” or “similar activity” when the functional portion or fragment displays no more than a 50% reduction in performance in a selected assay as compared to the parent or reference polypeptide (preferably no more than 20% or 10%, or no more than a log difference as compared to the parent or reference with regard to affinity).

As used herein, the term “engineered,” “recombinant,” or “non-natural” refers to an organism, microorganism, cell, nucleic acid molecule, or vector that includes at least one genetic alteration or has been modified by introduction of an exogenous or heterologous nucleic acid molecule, wherein such alterations or modifications are introduced by genetic engineering (i.e., human intervention). Genetic alterations include, for example, modifications introducing expressible nucleic acid molecules encoding functional RNA, proteins, fusion proteins or enzymes, or other nucleic acid molecule additions, deletions, substitutions, or other functional disruption of a cell's genetic material. Additional modifications include, for example, non-coding regulatory regions in which the modifications alter expression of a polynucleotide, gene, or operon.

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules (e.g., receptors, ligands, etc.) may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

In certain embodiments, a nucleic acid molecule or portion thereof native to a host cell will be considered heterologous to the host cell if it has been altered or mutated, or a nucleic acid molecule native to a host cell may be considered heterologous if it has been altered with a heterologous expression control sequence or has been altered with an endogenous expression control sequence not normally associated with the nucleic acid molecule native to a host cell. In addition, the term “heterologous” can refer to a biological activity that is different, altered, or not endogenous to a host cell. As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof.

As used herein, the term “endogenous” or “native” refers to a polynucleotide, gene, protein, compound, molecule, or activity that is normally present in a host cell or a subject.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a protein (e.g., a heavy chain of an antibody), or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

The term “construct” refers to any polynucleotide that contains a recombinant nucleic acid molecule (or, when the context clearly indicates, a fusion protein of the present disclosure). A (polynucleotide) construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Vectors of the present disclosure also include transposon systems (e.g., Sleeping Beauty, see, e.g., Geurts et al., Mol. Ther. 8:108, 2003: Mites et al., Nat. Genet. 41:753, 2009). Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, “expression vector” or “vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself or deliver the polynucleotide contained in the vector into the genome without the vector sequence. In the present specification, “plasmid,” “expression plasmid,” “virus,” and “vector” are often used interchangeably.

The term “introduced” in the context of inserting a nucleic acid molecule into a cell, means “transfection”, “transformation,” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g. transfected mRNA).

In certain embodiments, polynucleotides of the present disclosure may be operatively linked to certain elements of a vector. For example, polynucleotide sequences that are needed to effect the expression and processing of coding sequences to which they are ligated may be operatively linked. Expression control sequences may include appropriate transcription initiation, termination, promoter, and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

In certain embodiments, the vector comprises a plasmid vector or a viral vector (e.g., a lentiviral vector or a γ-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox, and canarypox). Other viruses include, for example, Norwalk virus, togavirus, flavivirus, reoviruses, papovavirus, hepadnavirus, and hepatitis virus. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses.

“Lentiviral vectors” include HIV-based lentiviral vectors for gene delivery, which can be integrative or non-integrative, have relatively large packaging capacity, and can transduce a range of different cell types. Lentiviral vectors are usually generated following transient transfection of three (packaging, envelope, and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing transgenes are known in the art and have been previous described, for example, in: U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors that can be used with the compositions and methods of this disclosure include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

Plasmid vectors, including DNA-based antibody or antigen-binding fragment-encoding plasmid vectors for direct administration to a subject, are described further herein.

As used herein, the term “host” refers to a cell or microorganism targeted for genetic modification with a heterologous nucleic acid molecule to produce a polypeptide of interest (e.g., an antibody of the present disclosure).

A host cell may include any individual cell or cell culture which may receive a vector or the incorporation of nucleic acids or express proteins. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

In the context of an influenza infection, a “host” refers to a cell or a subject infected with the influenza.

“Antigen” or “Ag”, as used herein, refers to an immunogenic molecule that provokes an immune response. This immune response may involve antibody production, activation of specific immunologically-competent cells, activation of complement, antibody dependent cytotoxicity, or any combination thereof. An antigen (immunogenic molecule) may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, stool samples, cells, biological fluids, or combinations thereof. Antigens can be produced by cells that have been modified or genetically engineered to express an antigen. Antigens can also be present in an influenza NA antigen, such as present in a virion, or expressed or presented on the surface of a cell infected by the influenza.

The term “epitope” or “antigenic epitope” includes any molecule, structure, amino acid sequence, or protein determinant that is recognized and specifically bound by a cognate binding molecule, such as an immunoglobulin, or other binding molecule, domain, or protein. Epitopic determinants generally contain chemically active surface groupings of molecules, such as amino acids or sugar side chains, and can have specific three-dimensional structural characteristics, as well as specific charge characteristics. Where an antigen is or comprises a peptide or protein, the epitope can be comprised of consecutive amino acids (e.g., a linear epitope), or can be comprised of amino acids from different parts or regions of the protein that are brought into proximity by protein folding (e.g., a discontinuous or conformational epitope), or non-contiguous amino acids that are in close proximity irrespective of protein folding.

### Antibodies, Antigen-Binding Fragments, and Compositions

In one aspect, the present disclosure provides an isolated anti-influenza neuraminidase (NA) antibody, or an antigen-binding fragment thereof. In certain embodiments, the antibody or antigen-binding fragment is capable of binding to a neuraminidase (NA) from: (i) an influenza A virus (IAV), wherein the IAV comprises a Group 1 IAV, a Group 2 IAV, or both; and (ii) an influenza B virus (IBV).

In certain embodiments, an antibody or antigen-binding fragment of the present disclosure associates with or unites with a NA while not significantly associating or uniting with any other molecules or components in a sample.

In certain embodiments, an antibody or antigen-binding fragment of the present disclosure specifically binds to a IAV NA. As used herein, “specifically binds” refers to an association or union of an antibody or antigen-binding fragment to an antigen with an affinity or Kₐ (i.e., an equilibrium association constant of a particular binding interaction with units of 1/M) equal to or greater than 10⁵ M⁻¹ (which equals the ratio of the on-rate [Kₒₙ] to the off rate [K_{off}] for this association reaction), while not significantly associating or uniting with any other molecules or components in a sample. Alternatively, affinity may be defined as an equilibrium dissociation constant (Kₐ) of a particular binding interaction with units of M (e.g., 10⁻⁵ M to 10⁻¹³ M). Antibodies may be classified as “high-affinity” antibodies or as “low-affinity” antibodies. “High-affinity” antibodies refer to those antibodies having a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low-affinity” antibodies refer to those antibodies having a Kₐ of up to 10⁷ M⁻¹, up to 10⁶ M⁻¹, up to 10⁵ M⁻¹. Alternatively, affinity may be defined as an equilibrium dissociation constant (Kₐ) of a particular binding interaction with units of M (e.g., 10⁻⁵ M to 10⁻¹³ M).

A variety of assays are known for identifying antibodies of the present disclosure that bind a particular target, as well as determining binding domain or binding protein affinities, such as Western blot, ELISA (e.g., direct, indirect, or sandwich), analytical ultracentrifugation, spectroscopy, biolayer interferometry, and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent). Assays for assessing affinity or apparent affinity or relative affinity are also known.

In certain examples, binding can be determined by recombinantly expressing an influenza NA antigen in a host cell (e.g., by transfection) and immunostaining the (e.g., fixed, or fixed and permeabilized) host cell with antibody and analyzing binding by flow cytometry (e.g., using a ZE5 Cell Analyzer (BioRad®) and FlowJo software (TreeStar). In some embodiments, positive binding can be defined by differential staining by antibody of influenza NA-expressing cells versus control (e.g., mock) cells.

In some embodiments an antibody or antigen-binding fragment of the present disclosure binds to an influenza NA protein, as measured using biolayer interferometry, or by surface plasmon resonance.

Certain characteristics of presently disclosed antibodies or antigen-binding fragments may be described using IC50 or EC50 values. In certain embodiments, the IC50 is the concentration of a composition (e.g., antibody) that results in half-maximal inhibition of the indicated biological or biochemical function, activity, or response. In certain embodiments, the EC50 is the concentration of a composition that provides the half-maximal response in the assay. In some embodiments, e.g., for describing the ability of a presently disclosed antibody or antigen-binding fragment to neutralize infection by influenza, IC50 and EC50 are used interchangeably.

In certain embodiments, an antibody of the present disclosure is capable of neutralizing infection by influenza. As used herein, a “neutralizing antibody” is one that can neutralize, i.e., prevent, inhibit, reduce, impede, or interfere with, the ability of a pathogen to initiate and/or perpetuate an infection in a host. The terms “neutralizing antibody” and “an antibody that neutralizes” or “antibodies that neutralize” are used interchangeably herein. In any of the presently disclosed embodiments, the antibody or antigen-binding fragment can be capable of preventing and/or neutralizing an influenza infection in an in vitro model of infection and/or in an in vivo animal model of infection and/or in a human.

In certain embodiments, the antibody, or antigen-binding fragment thereof, is human, humanized, or chimeric.

In certain embodiments, (i) the Group 1 IAV NA comprises a N1, a N4, a N5, and/or a N8; and/or (ii) the Group 2 IAV NA comprises a N2, a N3, a N6, a N7, and/or a N9. In some embodiments: (i) the N1 is a N1 from any one or more of: A/California/07/2009, A/California/07/2009 I223R/H275Y, A/California/07/2009 Q250S, A/Swine/Jiangsu/J004/2018, A/Swine/Hebei/2017, A/Stockholm/18/2007, A/Brisbane/02/2018, A/Michigan/45/2015, A/Mississippi/3/2001, A/Netherlands/603/2009, A/Netherlands/602/2009, A/Vietnam/1203/2004, A/Vietnam/1203/2004 S247R, A/Vietnam/1203/2004 I223R, A/Vietnam/1203/2004 R152I, A/Vietnam/1203/2004 D199N, A/G4/SW/Shangdong/1207/2016, A/G4/SW/Henan/SN13/2018, A/Mink/Spain/2022, and A/New Jersey/8/1976; (ii) the N4 is from A/mallard duck/Netherlands/30/2011; (iii) the N5 is from A/aquatic bird/Korea/CN5/2009; (iv) the N8 is from A/harbor seal/New Hampshire/179629/2011 A/chicken/Russia/3-29/2020; (v) the N2 is a N2 from any one or more of: A/Washington/01/2007, A/HongKong/68, A/South Australia/34/2019, A/Switzerland/8060/2017, A/Singapore/INFIMH-16-0019/2016, A/Switzerland/9715293/2013, A/Leningrad/134/17/57, A/Florida/4/2006, A/Netherlands/823/1992, A/Norway/466/2014, A/Switzerland/8060/2017, A/Texas/50/2012, A/Victoria/361/2011, A/HongKong/2671/2019, A/HongKong/2671/2019 K431E, A/SW/Mexico/SG1444/2011, A/Tanzania/205/2010, A/Aichi/2/1968, A/Bilthoven/21793/1972, A/Netherlands/233/1982, A/Shanghai/11/1987, A/Nanchang/933/1995, A/Fukui/45/2004, A/Brisbane/10/2007, A/Tasmania/503/2020, A/Cambodia/2020, A/Perth/16/2009, A/Kansas/14/2017, A/Swine/Kansas/2021, A/Canine/Korea/VC378/2012, and A/Canine/Indiana/003018/2016 (vi) the N3 is from A/Canada/rv504/2004 and A/chicken/Jalisco/PAVX17170/2017; (v) the N6 is from A/swine/Ontario/01911/1/99, A/Ck/Suzhou/j6/2019, and A/Hangzhou/01/2021; (vi) the N7 is from A/Netherlands/078/03 and A/Ck/621572/03; and/or (vii) the N9 is a N9 from any one or more of: A/Anhui/2013 and A/Hong Kong/56/2015. In certain embodiments, the IBV NA is a NA from any one or more of: B/Lee/10/1940 (Ancestral); B/Brisbane/60/2008 (Victoria); B/Malaysia/2506/2004 (Victoria); B/Malaysia/3120318925/2013 (Yamagata); B/Wisconsin/l/2010 (Yamagata); B/Yamanashi/166/1998 (Yamagata); B/Brisbane/33/2008; B/Colorado/06/2017; B/Hubei-wujiang/158/2009; B/Massachusetts/02/2012; B/Netherlands/234/2011; B/Perth/211/2001; B/Phuket/3073/2013; B/Texas/06/2011 (Yamagata); B/Perth/211/2011; B/HongKong/05/1972; B/Harbin/7/1994 (Victoria); B/Washington/02/2019 (Victoria); B/Victoria/504/2000 (Yamagata); B/Victoria/2/87; B/Victoria/2/87-lineage; B/Yamagata/16/88; and B/Yamagata/16/88-lineage.

In certain embodiments, the antibody or antigen-binding fragment is capable of binding to each of: (i) a Group 1 IAV NA; (ii) a Group 2 IAV NA; and (iii) a IBV NA with an EC₅₀ in a range of from about 0.1 μg/mL to about 50 μg/mL, or in a range of from about 0.1 μg/mL to about 2 μg/mL, or in a range of from 0.1 μg/mL to about 10 μg/mL, or in a range of from 2 μg/mL to about 10 μg/mL, or in a range of from about 0.4 μg/mL to about 50 μg/mL, or in a range of from about 0.4 μg/mL to about 2 μg/mL, or in a range of from 0.4 μg/mL to about 10 μg/mL, or in a range of from 2 μg/mL to about 10 μg/mL, or in a range of from 0.4 μg/mL to about 1 μg/mL, or 0.4 μg/mL or less.

In certain embodiments, the antibody or antigen-binding fragment is capable of binding to: (i) the Group 1 IAV NA with an EC₅₀ in a range of: from about 0.4 μg/mL to about 50 μg/mL, from about 0.4 μg/mL to about 10 μg/mL, from about 0.4 μg/mL to about 2 μg/mL, from about 2 μg/mL to about 50 μg/mL, from about 2 μg/mL to about 10 μg/mL, or from about 10 μg/mL to about 50 μg/mL; (ii) the Group 2 IAV NA with an EC₅₀ in a range from about 0.4 μg/mL to about 50 μg/mL, or from about 0.4 μg/mL to about 10 μg/mL, or from about 0.4 μg/mL to about 2 μg/mL, or from about 2 μg/mL to about 50 μg/mL, or from about 2 μg/mL to about 10 μg/mL, or from about 10 μg/mL to about 50 μg/mL; and/or (iii) the IBV NA with an EC₅₀ of about 0.4 μg/mL, or in a range from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL. In further embodiments, the antibody or antigen-binding fragment is capable of binding to: (i) a N1 with an EC₅₀ of about 0.4 μg/mL, or in a range from about 0.4 μg/mL to about 50 μg/mL, or in a range of: from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL; (ii) a N4 with an EC₅₀ of about 0.4 μg/mL, or in a range of: from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL; (iii) a N5 with an EC₅₀ in a range of: from about 0.4 μg/mL to about 2 μg/mL; (iv) a N8 with an EC₅₀ of about 50 μg/mL; (v) a N2 with an EC₅₀ in a range of: from about 0.4 μg/mL to about 20 μg/mL, or from about 0.4 μg/mL to about 10 μg/mL, or from about 0.4 μg/mL to about 2 μg/mL, from about 1 μg/mL to about 10 μg/mL, or from about 1 μg/mL to about 20 μg/mL, or from about 1 μg/mL to about 5 μg/mL, or about 1, 2, 3, 4, 5, 6, 7, 8, 9, 10, 15, or 20 μg/mL; (vi) a N3 with an EC₅₀ of about 0.4 μg/mL, or in a range of: from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL; (vii) a N6 with an EC₅₀ of about 0.4 μg/mL, or in a range of from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL; (viii) a N7 with an EC₅₀ in a range of: from about 2 μg/mL to about 50 μg/mL; (ix) a N9 with an EC₅₀ of about 0.4 μg/mL, or in a range of: from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL; and/or (xi) a IBV NA with an EC₅₀ of about 0.4 μg/mL, or in a range of: from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL.

In certain embodiments, the antibody or antigen-binding fragment is capable of binding to: (i) one or more of: N1 A/California/07/2009, N1 A/California/07/2009 I223R/H275Y, N1 A/Swine/Jiangsu/J004/2008, N1 A/Stockholm/18/2007, N4 A/mallard duck/Netherlands/30/2011, N5 A/aquatic bird/Korea/CN5/2009, N2 A/Hong Kong/68, N2 A/Leningrad/134/17/57, N3 A/Canada/rv504/2004, N6 A/Swine/Ontario/01911/1/99, N9 A/Anhui/1/2013, B/Lee/10/1940 (Ancestral), B/Brisbane/60/2008 (Victoria), B/Malaysia/2506/2004 (Victoria), B/Malaysia/3120318925/2013 (Yamagata), B/Wisconsin/1/2010 (Yamagata), and B/Yamanashi/166/1998 (Yamagata), with an EC₅₀ of about 0.4 μg/mL, or in a range from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL; (ii) N5 A/aquatic bird/Korea/CN5/2009 with an EC₅₀ of about 2 μg/mL, or in a range from about 2 μg/mL to about 10 μg/mL; (iii) N8 A/harbor seal/New Hampshire/179629/2011 with an EC₅₀ of about 50 μg/mL; (iv) N2 A/Washington/01/2007 with an EC₅₀ in a range from about 2 μg/mL to about 10 μg/mL; (v) N7 A/Netherlands/078/03 with an EC₅₀ in a range from about 2 μg/mL to about 50 μg/mL; (vi) N2 A/South Australia/34/2019 with an EC₅₀ in a range from about 0.4 μg/mL to about 50 μg/mL; (vii) N2 A/Switzerland/8060/2017 with an EC₅₀ in a range from about 9.5 μg/mL to about 3.8 μg/mL; (viii) N2 A/Singapore/INFIMH-16-0019/2016 with an EC₅₀ in a range from about 18.4 μg/mL to about 2.2 μg/mL; (iv) N2 A/Switzerland/9715293/2013 with an EC₅₀ in a range from about 1.6 μg/mL to about 1.2 μg/mL; and/or (v) N1 A/Swine/Jiangsu/J004/2018 with an EC₅₀ in a range from about 0.4 μg/mL to about 50 μg/mL, or about 0.4, about 2, about 10, or about 50 μg/mL. In certain embodiments, the NA is expressed on the surface of a host cell (e.g., a CHO cell) and binding to NA is according to flow cytometry.

In certain embodiments, the antibody or antigen-binding fragment is capable of binding to the NA with a KD of less than 1.0E-12 M, less than 1.0E-11 M, less than 1.0 E-11 M, or of 1.0E-12M or less, 1.0E-11M or less, or 1.0E-10 or less, or with a KD between 1.0E-10 and 1.0E-13, or with a KD between 1.0E-11 and 1.0E-13, wherein, optionally, the binding is as assessed by biolayer interferometry (BLI).

In certain embodiments, the NA is a N1, a N2, and/or a N9. In certain embodiments, the antibody or antigen-binding fragment is capable of binding to: (1) a NA epitope that comprises any one or more of the following amino acids (N1 NA numbering): R368, R293, E228, E344, S247, D198, D151, R118; and/or (2) a NA epitope that comprises any one or more of the following amino acids (N2 NA numbering): R371, R292, E227, E344, S247, D198, D151, R118. It will be understood that the antibodies and antigen-binding fragments may also bind to influenza neuraminidases which may not follow N1 or N2 amino acid numbering conventions; amino acids of these epitopes may correspond to herein-indicated N1 or N2 amino acid residues, such as by being the same amino acid residue at an equivalent (e.g., by alignment, 3-D structure, conservation, or combinations of these) but differently numbered, position in the NA. Accordingly, reference to N1 or N2 numbering will be understood as the amino acid corresponding to the enumerated amino acid. An example showing N1 vs N2 position numbering (using H1N1_California.07.2009 and H3N2_NewYork.392.2004) is provided in Table 3.

In certain embodiments, the antibody or antigen-binding fragment is capable of binding to: (1) a NA epitope that comprises the amino acids R368, R293, E228, D151, and R118 (N1 NA numbering); and/or (2) a NA epitope that comprises the amino acids R371, R292, E227, D151, and R118 (N2 NA numbering). In certain embodiments, the antibody or antigen-binding fragment is capable of binding to an epitope comprised in or comprising a NA active site (as described herein, the NA active site comprises functional amino acids that form the catalytic core and directly contact sialic acid, as well as structural amino acids that form the active site framework), wherein, optionally, the NA active site comprises the following amino acids (N2 numbering): R118, D151, R152, R224, E276, R292, R371, Y406, E119, R156, W178, S179, D/N198, I222, E227, H274, E277, D293, E425. In certain embodiments, R118, D151, R152, R224, E276, R292, R371, and Y406 form the catalytic core and directly contact sialic acid. In certain embodiments, E119, R156, W178, S179, D/N198, I222, E227, H274, E277, D293, and E425 form the active site framework.

In certain embodiments, the epitope comprises or further comprises any one or more of the following NA amino acids (N2 numbering): E344, E227, S247, and D198. In certain embodiments, the antibody or antigen-binding fragment is capable of binding to a NA comprising a S245N amino acid mutation and/or a E221D amino acid mutation (N2 numbering).

In certain embodiments, the NA comprises an IBV NA. In certain embodiments, the antibody or antigen-binding fragment is capable of binding to an IBV NA epitope that comprises any one or more of the following amino acids (IBV numbering; e.g., as for FluB Victoria and FluB Yamagata): R116, D149, E226, R292, and R374. In some embodiments, the epitope comprises the amino acids R116, D149, E226, R292, and R374.

In certain embodiments, the antibody or antigen-binding fragment is capable of inhibiting a sialidase activity of (i) an IAV NA, wherein the IAV NA comprises a Group 1 IAV NA, a Group 2 IAV NA, or both, and/or of (ii) an IBV NA, in an in vitro model of infection, an in vivo animal model of infection, and/or in a human. In further embodiments: (i) the Group 1 IAV NA comprises a H1N1 and/or a H5N1; (ii) the Group 2 IAV NA comprises a H3N2 and/or a H7N9; and/or (iii) the IBV NA comprises one or more of: B/Lee/10/1940 (Ancestral); B/HongKong/05/1972; B/Taiwan/2/1962 (Ancestral); B/Brisbane/33/2008 (Victoria); B/Brisbane/60/2008 (Victoria); B/Malaysia/2506/2004 (Victoria); B/New York/1056/2003 (Victoria); B/Florida/4/2006 (Yamagata); B/Jiangsu/10/2003 (Yamagata); B/Texas/06/2011 (Yamagata); B/Perth/211/2011; B/Harbin/7/1994 (Victoria); B/Colorado/06/2017 (Victoria); B/Washington/02/2019 (Victoria); B/Perth/211/2001 (Yamagata); B/Hubei-wujiagang/158/2009 (Yamagata); B/Wisconsin/01/2010 (Yamagata); B/Massachusetts/02/2012 (Yamagata); B/Phuket/3073/2013 (Yamagata); and B/Victoria/504/2000 (Yamagata).

In certain embodiments, the antibody or antigen-binding fragment is capable of inhibiting a sialidase activity by: a Group 1 IAV NA; a Group 2 IAV NA; and/or a IBV NA, with an IC50 in a range of: from about 0.0008 μg/mL to about 4 μg/mL, from about 0.0008 μg/mL to about 3 μg/mL, from about 0.0008 μg/mL to about 2 μg/mL, from about 0.0008 μg/mL to about 1 μg/mL, from about 0.0008 μg/mL to about 0.9 μg/mL, from about 0.0008 μg/mL to about 0.8 μg/mL, from about 0.0008 μg/mL to about 0.7 μg/mL, from about 0.0008 μg/mL to about 0.6 μg/mL, from about 0.0008 μg/mL to about 0.5 μg/mL, from about 0.0008 μg/mL to about 0.4 μg/mL, from about 0.0008 μg/mL to about 0.3 μg/mL, from about 0.0008 μg/mL to about 0.2 μg/mL, from about 0.0008 μg/mL to about 0.1 μg/mL, from about 0.0008 μg/mL to about 0.09 μg/mL, from about 0.0008 μg/mL to about 0.08 μg/mL, from about 0.0008 μg/mL to about 0.07 μg/mL, from about 0.0008 μg/mL to about 0.06 μg/mL, about 0.0008 μg/mL to about 0.05 μg/mL, about 0.0008 μg/mL to about 0.04 μg/mL, about 0.0008 μg/mL to about 0.03 μg/mL, about 0.0008 μg/mL to about 0.02 μg/mL, about 0.0008 μg/mL to about 0.01 μg/mL, from 0.002 μg/mL to about 4 μg/mL, from about 0.001 μg/mL to 50 μg/mL, from about 0.1 μg/mL to about 30 μg/mL, from about 0.1 μg/mL to about 20 μg/mL, from about 0.1 μg/mL to about 10 μg/mL, from about 0.1 μg/mL to about 9 μg/mL, from about 0.1 μg/mL to about 8 μg/mL, from about 0.1 μg/mL to about 7 μg/mL, from about 0.1 μg/mL to about 6 μg/mL, from about 0.1 μg/mL to about 5 μg/mL, from about 0.1 μg/mL to about 4 μg/mL, from about 0.1 μg/mL to about 3 μg/mL, from about 0.1 μg/mL to about 2 μg/mL, from about 0.1 μg/mL to about 1 μg/mL, from about 0.1 μg/mL to about 0.9 μg/mL, from about 0.1 μg/mL to about 0.8 μg/mL, from about 0.1 μg/mL to about 0.7 μg/mL, from about 0.1 μg/mL to about 0.6 μg/mL, from about 0.1 μg/mL to about 0.5 μg/mL, from about 0.1 μg/mL to about 0.4 μg/mL, from about 0.1 μg/mL to about 0.3 μg/mL, from about 0.1 μg/mL to about 0.2 μg/mL, from about 0.8 μg/mL to about 30 μg/mL, from about 0.8 μg/mL to about 20 μg/mL, from about 0.8 μg/mL to about 10 μg/mL, from about 0.8 μg/mL to about 9 μg/mL, from about 0.8 μg/mL to about 8 μg/mL, from about 0.8 μg/mL to about 7 μg/mL, from about 0.8 μg/mL to about 6 μg/mL, from about 0.8 μg/mL to about 5 μg/mL, from about 0.8 μg/mL to about 4 μg/mL, from about 0.8 μg/mL to about 3 μg/mL, from about 0.8 μg/mL to about 2 μg/mL, of from about 0.8 μg/mL to about 1 μg/mL, or of about 0.1 μg/mL, about 0.2 μg/mL, about 0.3 μg/mL, about 0.4 μg/mL, about 0.5 μg/mL, about 0.6 μg/mL, about 0.7 μg/mL, about 0.8 μg/mL, about 0.9 μg/mL, about 1.0 μg/mL, about 1.5 μg/mL, about 2.0 μg/mL, about 2.5 μg/mL, about 3.0 μg/mL, about 3.5 μg/mL, about 4.0 μg/mL, about 4.5 μg/mL, about 5.0 μg/mL, about 5.5 μg/mL, about 6.0 μg/mL, about 6.5 μg/mL, about 7.0 μg/mL, about 7.5 μg/mL, about 8.0 μg/mL, about 8.5 μg/mL, about 9.0 μg/mL, about 10 μg/mL, about 11 μg/mL, about 12 μg/mL, about 13 μg/mL, about 14 μg/mL, about 15 μg/mL, about 16 μg/mL, about 17 μg/mL, about 18 μg/mL, about 19 μg/mL, about 20 μg/mL, about 25 μg/mL, and/or about 30 μg/mL. In further embodiments, the antibody or antigen-binding fragment is capable of inhibiting NA sialidase activity of one or more Group 1 and/or Group 2 IAV, and/or of one or more IBV, with an IC50 in a range of: from about 0.00001 μg/ml to about 25 μg/ml, or about 0.0001 μg/ml to about 10 μg/ml, or about 0.0001 μg/ml to about 1 μg/ml, or about 0.0001 μg/ml to about 0.1 μg/ml, or about 0.0001 μg/ml to about 0.01 μg/ml, or about 0.0001 μg/ml to about 0.001 μg/ml, or about 0.0001 μg/ml to about 0.0001 μg/ml, or about 0.0001 μg/ml to about 25 μg/ml, or about 0.0001 μg/ml to about 10 μg/ml, or about 0.0001 μg/ml to about 1 μg/ml, or about 0.0001 μg/ml to about 0.1 μg/ml, or about 0.0001 μg/ml to about 0.01 μg/ml, or about 0.001 μg/ml to about 25 μg/ml, or about 0.001 μg/ml to about 10 μg/ml, or about 0.001 μg/ml to about 1 μg/ml, or about 0.001 μg/ml to about 0.1 μg/ml, or about 0.001 μg/ml to about 0.01 μg/ml, or about 0.01 μg/ml to about 25 μg/ml, or about 0.01 μg/ml to about 10 μg/ml, or about 0.01 μg/ml to about 1 μg/ml, or about 0.01 μg/ml to about 0.1 μg/ml, or about 1 μg/ml to about 25 μg/ml, or about 1 μg/ml to about 10 μg/ml, or of about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 μg/ml.

In certain embodiments, the antibody or antigen-binding fragment is capable of activating a human FcγRIIIa. In further embodiments, activation is as determined using a host cell (optionally, a Jurkat cell) comprising: (i) the human FcγRIIIa (optionally, a F158 allele); and (ii) a NFAT expression control sequence operably linked to a sequence encoding a reporter, such as a luciferase reporter, following incubation (e.g., of 23 hours) of the antibody or antigen-binding fragment with a target cell (e.g., a A549 cell) infected with a IAV. In still further embodiments, activation is as determined following an incubation (optionally, for about 23 hours) of the antibody or antigen-binding fragment with the target cell infected with a H1N1 IAV, wherein, optionally, the H1N1 IAV is A/PR8/34, and/or wherein, optionally, the infection has a multiplicity of infection (MOI) of 6.

In certain embodiments, the antibody or antigen-binding fragment is capable of neutralizing infection by an IAV and/or an IBV. In certain embodiments, the IAV and/or the IBV is antiviral-resistant, wherein, optionally, the antiviral is oseltamivir. In certain embodiments, the IAV comprises a N1 NA that comprises the amino acid mutation(s): H275Y; E119D+H275Y; S247N+H275Y; I222V; and/or N294S wherein, optionally, the IAV comprises CA09 or A/Aichi. In certain embodiments, the IAV comprises a N2 NA that comprises the amino acid mutation(s) E119V, Q136K, and/or R292K. In certain embodiments, the IAV comprises a N1 NA that comprises the amino acid mutation(s): S247R, I223R, R152I, D199N, and/or Q250S, wherein, optionally, the IAV comprises A/Vietnam/1203/2004 or A/California/7/2009. In certain embodiments, the IAV comprises a N2 NA that comprises the amino acid mutation K431E, wherein optionally, the IAV comprises A/Hong Kong/2671/2019.

In certain embodiments, the antibody or antigen-binding fragment is capable of treating and/or preventing (i) an IAV infection and/or (ii) an IBV infection in a subject. In certain embodiments, the antibody or antigen-binding fragment is capable of treating and/or attenuating an infection by: (i) a H1N1 virus, wherein, optionally, the H1N1 virus comprises A/PR8/34; and/or (ii) a H3N2 virus, wherein, optionally, the H3N2 virus optionally comprises A/Hong Kong/68. In certain embodiments, the antibody or antigen-binding fragment is capable of preventing weight loss in a subject infected by the IAV and/or IBV, optionally for (i) up to 15 days, or (ii) more than 15 days, following administration of an effective amount of the antibody or antigen-binding fragment.

In certain embodiments, the antibody or antigen-binding fragment is capable of preventing a loss in body weight of greater than 10% in a subject having an IAV infection and/or an IBV infection, as determined by reference to the subject's body weight just prior to the IAV and/or IBV infection.

In certain embodiments, the antibody or antigen-binding fragment is capable extending survival of a subject having an IAV infection and/or an IBV infection.

In certain embodiments, the antibody or antigen-binding fragment has an in vivo half-life in a mouse (e.g., a tg32 mouse): (i) in a range of: from about 10 days to about 14 days, about 10.2 days to about 13.8 days, about 10.5 days to about 13.5 days, about 11 days to about 13 days, about 11.5 days to about 12.5 days, between 10 days and 14 days, or between 10.5 days and 13.5 days, or between 11 days and 13 days, or of about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, or 14.0 days; or (ii) in a range of: from about 12 days to about 16 days, about 12.5 days to 15.5 days, about 13 days to 15 days, about 13.5 days to about 14.5 days, or between 12 days and 16 days, or between 13 days and 15 days, or between 13.5 days and 14.5 days, or of about 12.0, 12.1, 12.2, 12.3, 12.4, 12.5, 12.6, 12.7, 12.8, 12.9, 13.0, 13.1, 13.2, 13.3, 13.4, 13.5, 1.36, 13.7, 13.8, 13.9, 14.0, 14.1, 14.2, 14.3, 14.4, 14.5, 14.6, 14.7, 14.8, 14.9, 15.0 15.1, 15.2, 15.3, 15.4, 15.5, 1.56, 15.7, 15.8, 15.9, or 16.0 days.

Terms understood by those in the art of antibody technology are each given the meaning acquired in the art, unless expressly defined differently herein. For example, the term “antibody” refers to an intact antibody comprising at least two heavy (H) chains and two light (L) chains inter-connected by disulfide bonds, as well as any antigen-binding portion or fragment of an intact antibody that has or retains the ability to bind to the antigen target molecule recognized by the intact antibody, such as an scFv, Fab, or Fab′2 fragment. Thus, the term “antibody” herein is used in the broadest sense and includes polyclonal and monoclonal antibodies, including intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific antibodies, diabodies, triabodies, tetrabodies, tandem di-scFv, and tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof (IgG1, IgG2, IgG3, IgG4), IgM, IgE, IgA, and IgD.

The terms “VL” or “VL” and “VH” or “VH” refer to the variable binding region from an antibody light chain and an antibody heavy chain, respectively. In certain embodiments, a VL is a kappa (κ) class (also “VK” herein). In certain embodiments, a VL is a lambda (λ) class. The variable binding regions comprise discrete, well-defined sub-regions known as “complementarity determining regions” (CDRs) and “framework regions” (FRs). The terms “complementarity determining region,” and “CDR,” are synonymous with “hypervariable region” or “HVR,” and refer to sequences of amino acids within antibody variable regions, which, in general, together confer the antigen specificity and/or binding affinity of the antibody, wherein consecutive CDRs (i.e., CDR1 and CDR2, CDR2 and CDR3) are separated from one another in primary structure by a framework region. There are three CDRs in each variable region (HCDR1, HCDR2, HCDR3; LCDR1, LCDR2, LCDR3; also referred to as CDRHs and CDRLs, respectively). In certain embodiments, an antibody VH comprises four FRs and three CDRs as follows: FR1-HCDR1-FR2-HCDR2-FR3-HCDR3-FR4; and an antibody VL comprises four FRs and three CDRs as follows: FR1-LCDR1-FR2-LCDR2-FR3-LCDR3-FR4. In general, the VH and the VL together form the antigen-binding site through their respective CDRs. In certain embodiments, one or more CDRs do not contact antigen and/or do not contribute energetically to antigen binding.

As used herein, a “variant” of a CDR refers to a functional variant of a CDR sequence having up to 1-3 amino acid substitutions (e.g., conservative or non-conservative substitutions), deletions, or combinations thereof.

Numbering of CDR and framework regions may be according to any known method or scheme, such as the Kabat, Chothia, EU, IMGT, Contact, North, Martin, and Aho numbering schemes (see, e.g., Kabat et al., “Sequences of Proteins of Immunological Interest, US Dept. Health and Human Services, Public Health Service National Institutes of Health, 1991, 5^{th} ed.; Chothia and Lesk, J. Mol. Biol. 196:901-917 (1987)); Lefranc et al., Dev. Comp. Immunol. 27:55, 2003; Honegger and Pluckthun, J. Mol. Bio. 309:657-670 (2001); North et al. J Mol Biol. (2011) 406:228-56; doi:10.1016/j.jmb.2010.10.030; Abhinandan and Martin, Mol Immunol. (2008) 45:3832-9. 10.1016/j.molimm.2008.05.022). The antibody and CDR numbering systems of these references are incorporated herein by reference. Equivalent residue positions can be annotated and for different molecules to be compared using Antigen receptor Numbering And Receptor Classification (ANARCI) software tool (2016, Bioinformatics 15:298-300). Accordingly, identification of CDRs of an exemplary variable domain (VH or VL) sequence as provided herein according to one numbering scheme is not exclusive of an antibody comprising CDRs of the same variable domain as determined using a different numbering scheme. For analyzing sequences in accordance with IMGT, imgt.org/IMGTindex/V-QUEST.php and imgt.org/IMGT_vquest/input may be used.

In certain embodiments, an antibody or an antigen-binding fragment of the present disclosure comprises a CDRH1, a CDRH2, a CDRH3, a CDRL1, a CDRL2, and a CDRL3, wherein each CDR is independently selected from a corresponding CDR of an NA-specific antibody as provided in Table 1 and/or Table 2. That is, all combinations of CDRs from NA-specific antibodies provided in Table 1 and/or Table 2 are contemplated.

In some embodiments, CDRs are in accordance with the IMGT numbering method.

The term “CL” refers to an “immunoglobulin light chain constant region” or a “light chain constant region,” i.e., a constant region from an antibody light chain. The term “CH” refers to an “immunoglobulin heavy chain constant region” or a “heavy chain constant region,” which is further divisible, depending on the antibody isotype, into CH1, CH2, and CH3 (IgA, IgD, IgG), or CH1, CH2, CH3, and CH4 domains (IgE, IgM). The Fc region of an antibody heavy chain is described further herein. In any of the presently disclosed embodiments, an antibody or antigen-binding fragment of the present disclosure comprises any one or more of CL, a CH1, a CH2, and a CH3. In any of the presently disclosed embodiments, an antibody or antigen-binding fragment of the present disclosure may comprise any one or more of CL, a CH1, a CH2, and a CH3. In certain embodiments, a CL comprises an amino acid sequence having 90%, 91%, 92%, 93%, 94%, 95%, 96%, 97%, 98%, 99%, or 100% identity (or similarity) to the amino acid sequence of SEQ ID NO:35. In certain embodiments, a CH1-CH2-CH3 comprises an amino acid sequence having 90%, 91%, 92%, 93%, 94%, 95%, 96%, 97%, 98%, 99%, or 100% identity (or similarity) to the amino acid sequence of any one of SEQ ID NOs:34, 35, 38, 70, and 74-95. It will be understood that, for example, production in a mammalian cell line can remove one or more C-terminal lysine of an antibody heavy chain (see, e.g., Liu et al. mAbs 6(5):1145-1154 (2014)). Accordingly, an antibody or antigen-binding fragment of the present disclosure can comprise a heavy chain, a CH1-CH3, a CH3, or an Fc polypeptide wherein a C-terminal lysine residue is present or is absent; in other words, encompassed are embodiments where the C-terminal residue of a heavy chain, a CH1-CH3, or an Fc polypeptide is not a lysine, and embodiments where a lysine is the C-terminal residue. In certain embodiments, a composition comprises a plurality of an antibody and/or an antigen-binding fragment of the present disclosure, wherein one or more antibody or antigen-binding fragment does not comprise a lysine residue at the C-terminal end of the heavy chain, CH1-CH3, or Fc polypeptide, and wherein one or more antibody or antigen-binding fragment comprises a lysine residue at the C-terminal end of the heavy chain, CH1-CH3, or Fc polypeptide.

In some embodiments, an antibody or antigen-binding fragment of the present disclosure can comprise a heavy chain, a CH1-CH3, a CH3, or an Fc polypeptide wherein a C-terminal glycine-lysine sequence (e.g., corresponding to the last two amino acids of SEQ ID NO:95) is present or is absent.

A “Fab” (fragment antigen binding) is the part of an antibody that binds to antigens and includes the variable region and CH1 of the heavy chain linked to the light chain via an inter-chain disulfide bond. Each Fab fragment is monovalent with respect to antigen binding, i.e., it has a single antigen-binding site. Pepsin treatment of an antibody yields a single large F(ab′)2 fragment that roughly corresponds to two disulfide linked Fab fragments having divalent antigen-binding activity and is still capable of cross-linking antigen. Both the Fab and F(ab′)2 are examples of “antigen-binding fragments.” Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them. Other chemical couplings of antibody fragments are also known.

Fab fragments may be joined, e.g., by a peptide linker, to form a single chain Fab, also referred to herein as “scFab.” In these embodiments, an inter-chain disulfide bond that is present in a native Fab may not be present, and the linker serves in full or in part to link or connect the Fab fragments in a single polypeptide chain. A heavy chain-derived Fab fragment (e.g., comprising, consisting of, or consisting essentially of VH+CH1, or “Fd”) and a light chain-derived Fab fragment (e.g., comprising, consisting of, or consisting essentially of VL+CL) may be linked in any arrangement to form a scFab. For example, a scFab may be arranged, in N-terminal to C-terminal direction, according to (heavy chain Fab fragment-linker-light chain Fab fragment) or (light chain Fab fragment-linker-heavy chain Fab fragment). Peptide linkers and exemplary linker sequences for use in scFabs are discussed in further detail herein.

“Fv” is a small antibody fragment that contains a complete antigen-recognition and antigen-binding site. This fragment generally consists of a dimer of one heavy- and one light-chain variable region domain in tight, non-covalent association. However, even a single variable domain (or half of an Fv comprising only three CDRs specific for an antigen) has the ability to recognize and bind antigen, although typically at a lower affinity than the entire binding site.

“Single-chain Fv” also abbreviated as “sFv” or “scFv”, are antibody fragments that comprise the VH and VL antibody domains connected into a single polypeptide chain. In some embodiments, the scFv polypeptide comprises a polypeptide linker disposed between and linking the VH and VL domains that enables the scFv to retain or form the desired structure for antigen binding. Such a peptide linker can be incorporated into a fusion polypeptide using standard techniques well known in the art. For a review of scFv, see Pluckthun in The Pharmacology of Monoclonal Antibodies, vol. 113, Rosenburg and Moore eds., Springer-Verlag, New York, pp. 269-315 (1994); Borrebaeck 1995, infra. In certain embodiments, the antibody or antigen-binding fragment comprises a scFv comprising a VH domain, a VL domain, and a peptide linker linking the VH domain to the VL domain. In particular embodiments, a scFv comprises a VH domain linked to a VL domain by a peptide linker, which can be in a VH-linker-VL orientation or in a VL-linker-VH orientation. Any scFv of the present disclosure may be engineered so that the C-terminal end of the VL domain is linked by a short peptide sequence to the N-terminal end of the VH domain, or vice versa (i.e., (N)VL(C)-linker-(N)VH(C) or (N)VH(C)-linker-(N)VL(C). Alternatively, in some embodiments, a linker may be linked to an N-terminal portion or end of the VH domain, the VL domain, or both.

Peptide linker sequences may be chosen, for example, based on: (1) their ability to adopt a flexible extended conformation; (2) their inability or lack of ability to adopt a secondary structure that could interact with functional epitopes on the first and second polypeptides and/or on a target molecule; and/or (3) the lack or relative lack of hydrophobic or charged residues that might react with the polypeptides and/or target molecule. Other considerations regarding linker design (e.g., length) can include the conformation or range of conformations in which the VH and VL can form a functional antigen-binding site. In certain embodiments, peptide linker sequences contain, for example, Gly, Asn and Ser residues. Other near neutral amino acids, such as Thr and Ala, may also be included in a linker sequence. Other amino acid sequences which may be usefully employed as linker include those disclosed in Maratea et al., Gene 40:39 46 (1985); Murphy et al., Proc. Natl. Acad. Sci. USA 83:8258 8262 (1986); U.S. Pat. Nos. 4,935,233, and 4,751,180. Other illustrative and non-limiting examples of linkers may include, for example, Glu-Gly-Lys-Ser-Ser-Gly-Ser-Gly-Ser-Glu-Ser-Lys-Val-Asp (Chaudhary et al., Proc. Natl. Acad. Sci. USA 87:1066-1070 (1990)) and Lys-Glu-Ser-Gly-Ser-Val-Ser-Ser-Glu-Gln-Leu-Ala-Gln-Phe-Arg-Ser-Leu-Asp (Bird et al., Science 242:423-426 (1988)) and the pentamer Gly-Gly-Gly-Gly-Ser when present in a single iteration or repeated 1 to 5 or more times, or more. Any suitable linker may be used, and in general can be about 3, 4, 5, 6, 7, 8, 9, 10, 11, 12, 13, 14, 15, 16, 17, 18, 19, 20, 21, 22, 1523, 24, 25, 26, 27, 28, 29, 30, 40, 50, 60, 70, 80, 90, 100 amino acids in length, or less than about 200 amino acids in length, and will preferably comprise a flexible structure (can provide flexibility and room for conformational movement between two regions, domains, motifs, fragments, or modules connected by the linker), and will preferably be biologically inert and/or have a low risk of immunogenicity in a human. ScFvs can be constructed using any combination of the VH and VL sequences or any combination of the CDRH1, CDRH2, CDRH3, CDRL1, CDRL2, and CDRL3 sequences disclosed herein. In some embodiments, linker sequences are not required; for example, when the first and second polypeptides have non-essential N-terminal amino acid regions that can be used to separate the functional domains and prevent steric interference.

During antibody development, DNA in the germline variable (V), joining (J), and diversity (D) gene loci may be rearranged and insertions and/or deletions of nucleotides in the coding sequence may occur. Somatic mutations may be encoded by the resultant sequence, and can be identified by reference to a corresponding known germline sequence. In some contexts, somatic mutations that are not critical to a desired property of the antibody (e.g., binding to a influenza NA antigen), or that confer an undesirable property upon the antibody (e.g., an increased risk of immunogenicity in a subject administered the antibody), or both, may be replaced by the corresponding germline-encoded amino acid, or by a different amino acid, so that a desirable property of the antibody is improved or maintained and the undesirable property of the antibody is reduced or abrogated. Thus, in some embodiments, the antibody or antigen-binding fragment of the present disclosure comprises at least one more germline-encoded amino acid in a variable region as compared to a parent antibody or antigen-binding fragment, provided that the parent antibody or antigen binding fragment comprises one or more somatic mutations. Variable region and CDR amino acid sequences of exemplary anti-NA antibodies of the present disclosure are provided in Table 1 herein.

Polynucleotide sequences and other information of these and related human IG alleles are available at, for example, IMGT.org (see e.g.).

In certain embodiments, an antibody or antigen-binding fragment comprises an amino acid modification (e.g., a substitution mutation) to remove an undesired risk of oxidation, deamidation, and/or isomerization.

Also provided herein are variant antibodies that comprise one or more amino acid alterations in a variable region (e.g., VH, VL, framework or CDR) as compared to a presently disclosed (“parent”) antibody, wherein the variant antibody is capable of binding to a NA antigen.

In certain embodiments, the VH comprises, consists essentially of, or consists of any VH amino acid sequence set forth in Table 1 and/or Table 2, and the VL comprises, consists essentially of, or consists of any VL amino acid sequence set forth in Table 1 and/or Table 2.

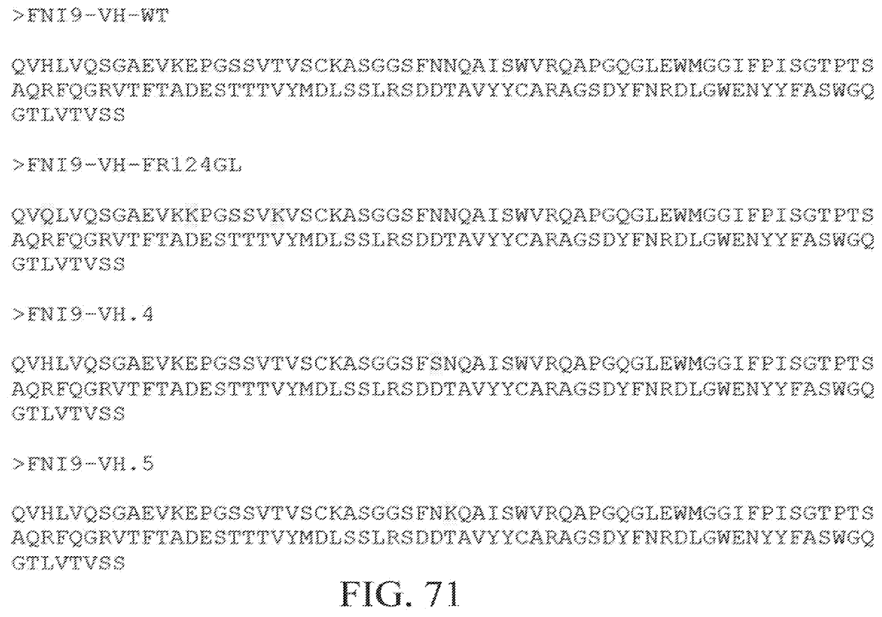

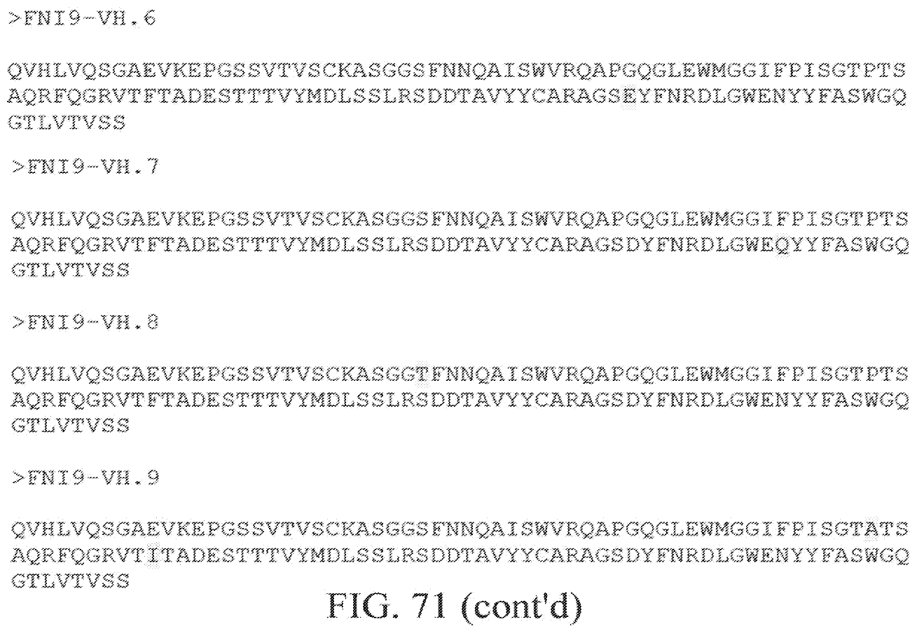

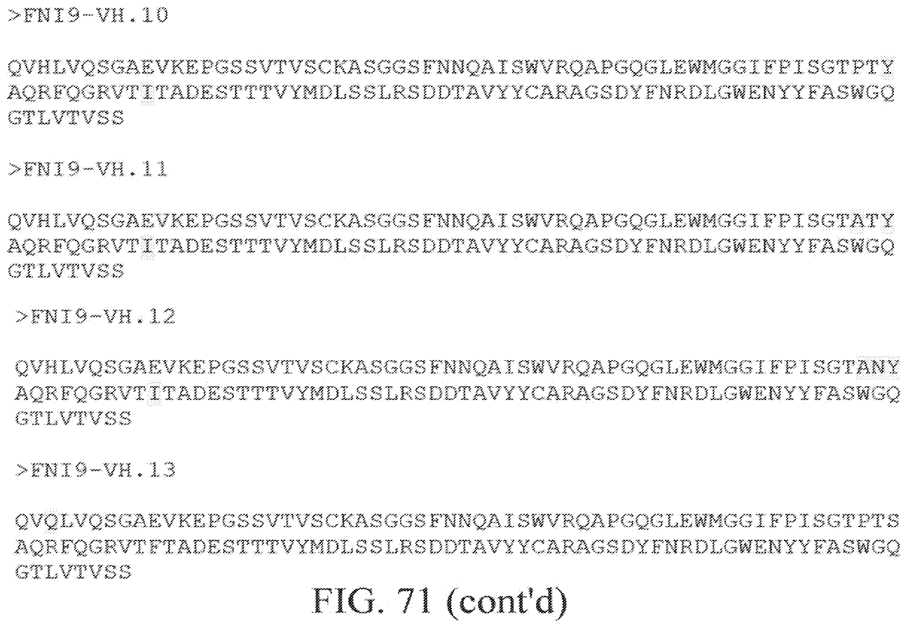

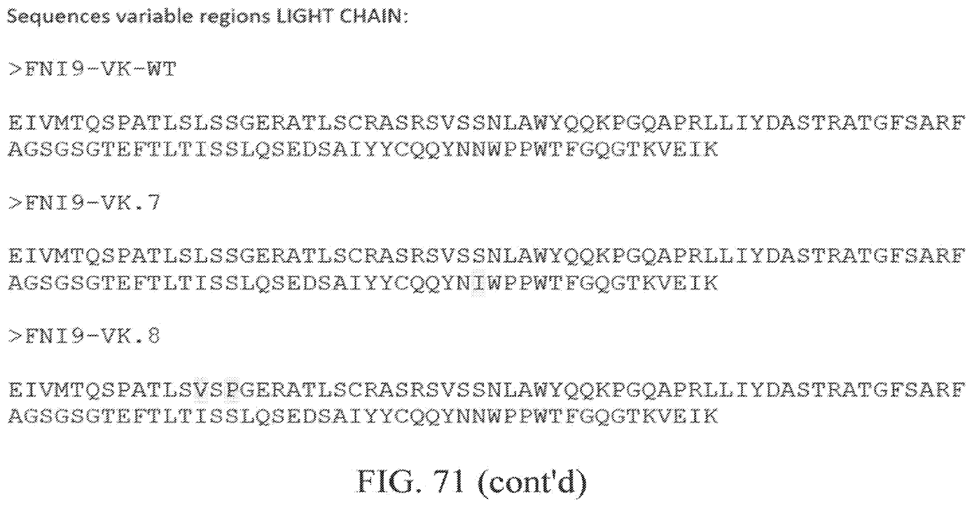

With reference to FIG. 71, in certain embodiments, an antibody or antigen-binding fragment is provided that comprises a VH of a FNI9 antibody shown in FIG. 71 and a VL of a FNI9 antibody shown in FIG. 71, provided that the antibody or antigen-binding fragment does not comprise the VH of FNI9-VH-WT and the VL of FNI9-VK-WT.

With reference to FIG. 71, in certain embodiments, an antibody or antigen-binding fragment is provided that comprises (i) a VH comprising the VH amino acid sequence of FNI9-VH-WT, FNI9-VH-FR124GL, FNI9-VH.4, FNI9-VH.5, FNI9-VH.6, FNI9-VH.7, FNI9-VH.8, FNI9-VH.9, FNI9-VH.10, FNI9-VH.11, FNI9-VH.12, or FNI9-VH.13, and (ii) a VL comprising the VL amino acid sequence of FNI9-VK.7.

With reference to FIG. 71, in certain embodiments, an antibody or antigen-binding fragment is provided that comprises (i) a VH comprising the VH amino acid sequence of FNI9-VH-WT, FNI9-VH-FR124GL, FNI9-VH.4, FNI9-VH.5, FNI9-VH.6, FNI9-VH.7, FNI9-VH.8, FNI9-VH.9, FNI9-VH.10, FNI9-VH.11, FNI9-VH.12, or FNI9-VH.13, and (ii) a VL comprising the VL amino acid sequence of FNI9-VK.8.

With reference to FIG. 71, in certain embodiments, an antibody or antigen-binding fragment is provided that comprises (i) a VH comprising the VH amino acid sequence of FNI9-VH-FR124GL, FNI9-VH.4, FNI9-VH.5, FNI9-VH.6, FNI9-VH.7, FNI9-VH.8, FNI9-VH.9, FNI9-VH.10, FNI9-VH.11, FNI9-VH.12, or FNI9-VH.13, and (ii) a VL comprising the VL amino acid sequence of FNI9-VK-WT.

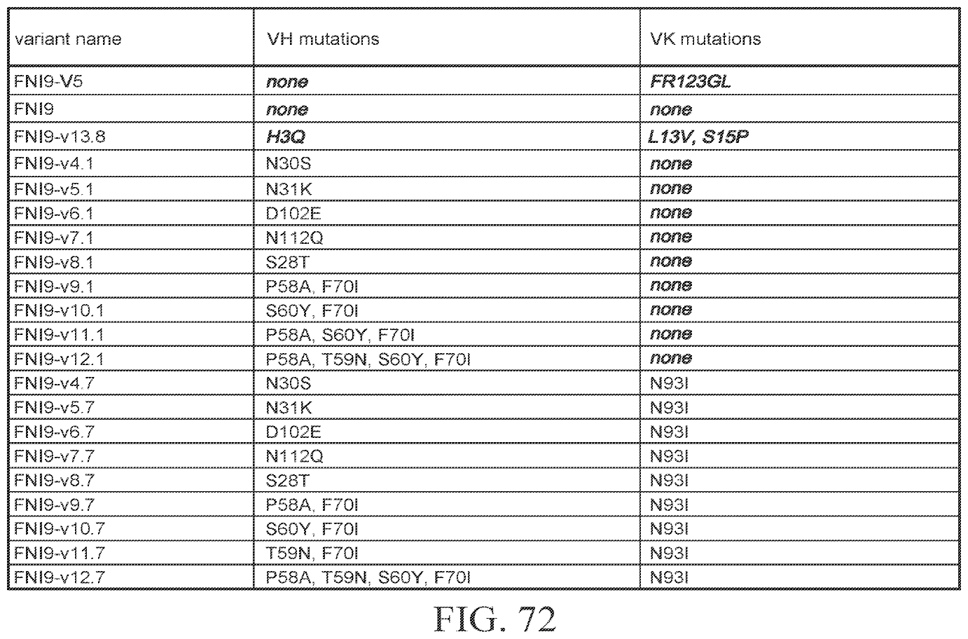

With reference to FIG. 72, in certain embodiments, an antibody or antigen-binding fragment is provided that comprises the VH and the VL of a FNI9 variant antibody shown in FIG. 72.

In certain embodiments, an antibody or antigen-binding fragment comprises CDRH1, CDRH2, CDRH3, CDRL1, CDRL2, and CDRL3, and optionally VH and VL, of antibody “FNI9-v8.1”, which differs from antibody FNI9 by a S28T mutation in the VH. Compared to FNI9, FNI9-v8.1 has improved production titer when expressed as recombinant IgG1 by transiently transfected host cells. FNI9-v8.1 also has a lower IC50 for inhibiting sialidase activity of certain N1 and N2 neuraminidases in a MUNANA assay, as compared to FNI9. FNI9-v8.1 also has a lower IC50 for inhibiting sialidase activity of a pseudovirus-derived neuraminidase in an ELLA assay, as compared to FNI9. FNI9-v8.1 also binds more strongly to N9_A_Anhui_2013 as compared to FNI9, assessed by flow cytometry. FNI9-v8.1 has higher affinity for certain IAV and IBV NA antigens (including glycan-bearing and non-glycan-bearing antigens) as compared to FNI9, as assessed by surface plasmon resonance. FNI9-v8.1 has improved in vitro inhibition of sialidase activity (reported as IC50 in ng/ml) for certain IAV and IBV NAs, as compared to FNI9.

FNI9-v8.1 comprises CDRH1, CDRH2, CDRH3, CDRL1, CDRL2, and CDRL3 amino acid sequences of SEQ ID NOs:55 (GGTFNNQA), 4 (IFPISGTP), 5 (ARAGSDYFNRDLGWENYYFAS), 9 (RSVSSN), 10 (DAS), and 11 (QQYNNWPPWT), respectively, and VH and VL amino acid sequences of SEQ ID NOs:54 and 8, respectively. It will be understood that “−v8.1” refers to a variant of FNI9 comprising the “v8” VH (SEQ ID NO:54) and the “v1” VL (SEQ ID NO:8, same VL as parental FNI9).

In some embodiments, an antibody or antigen-binding fragment is provided that comprises (i) a VH comprising or consisting of the amino acid sequence of any one of SEQ ID NOs:2, 45, 46, 48, 50, 52, 54, 56, 58, 60, 63, and 65, and (ii) a VL comprising or consisting of the VL amino acid sequence of SEQ ID NO:8.

In certain embodiments, an antibody or antigen-binding fragment is provided that comprises: (i) a VH comprising the amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSS (SEQ ID NO:54); and (ii) a VL comprising the amino acid sequence

(SEQ ID NO.: 8)
EIVMTQSPATLSLSSGERATLSCRASRSVSSNLAWYQQKPGQAPRLLIY
DASTRATGFSARFAGSGSGTEFTLTISSLQSEDSAIYYCQQYNNWPPWT
FGQGTKVEIK.

In certain embodiments, an antibody or antigen-binding fragment is provided that comprises: (i) a VH consisting essentially of the amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSS (SEQ ID NO:54); and (ii) a VL consisting essentially of the amino acid sequence

(SEQ ID NO.: 8)
EIVMTQSPATLSLSSGERATLSCRASRSVSSNLAWYQQKPGQAPRLLIY
DASTRATGFSARFAGSGSGTEFTLTISSLQSEDSAIYYCQQYNNWPPWT
FGQGTKVEIK.

In certain embodiments, an antibody or antigen-binding fragment is provided that comprises: (i) a VH consisting of the amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSS (SEQ ID NO:54); and (ii) a VL consisting of the amino acid sequence

(SEQ ID NO.: 8)
EIVMTQSPATLSLSSGERATLSCRASRSVSSNLAWYQQKPGQAPRLLIY
DASTRATGFSARFAGSGSGTEFTLTISSLQSEDSAIYYCQQYNNWPPWT
FGQGTKVEIK.

In certain embodiments, an antibody or antigen-binding fragment is provided that comprises: (i) a heavy chain comprising the VH amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSS (SEQ ID NO:54); and (ii) a light chain comprising the VL amino acid sequence

(SEQ ID NO.: 8)
EIVMTQSPATLSLSSGERATLSCRASRSVSSNLAWYQQKPGQAPRLLIY
DASTRATGFSARFAGSGSGTEFTLTISSLQSEDSAIYYCQQYNNWPPWT
FGQGTKVEIK.

In certain further embodiments, the antibody or antigen-binding fragment is an (e.g. human) IgG1 isotype and optionally comprises M428L and N434S mutations. In some embodiments, the light chain is an IgG1 kappa light chain.

In certain embodiments, an antibody or antigen-binding fragment is provided that comprises: (i) two heavy chains each comprising the VH amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSS (SEQ ID NO:54); and (ii) two light chains each comprising the VL amino acid sequence

(SEQ ID NO.: 8)
EIVMTQSPATLSLSSGERATLSCRASRSVSSNLAWYQQKPGQAPRLLIY
DASTRATGFSARFAGSGSGTEFTLTISSLQSEDSAIYYCQQYNNWPPWT
FGQGTKVEIK.

In certain further embodiments, the antibody or antigen-binding fragment is an (e.g. human) IgG1 isotype and optionally comprises M428L and N434S mutations. In some embodiments, the light chains are each an IgG1 kappa light chain. In certain embodiments, an antibody or antigen-binding fragment is provided that comprises: (i) a VH comprising CDRH1, CDRH2, and CDRH3 of the VH amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSS (SEQ ID NO:54), wherein, optionally, the CDRs are defined according to IMGT; and (ii) a VL comprising CDRL1, CDRL2, and CDRL3 of the VL amino acid sequence

(SEQ ID NO.: 8)
EIVMTQSPATLSLSSGERATLSCRASRSVSSNLAWYQQKPGQAPRLLIY
DASTRATGFSARFAGSGSGTEFTLTISSLQSEDSAIYYCQQYNNWPPWT
FGQGTKVEIK.

wherein, optionally, the CDRs are defined according to IMGT.

In certain embodiments, an antibody or antigen-binding fragment is provided that comprises: (i) a heavy chain comprising, in a VH, CDRH1, CDRH2, and CDRH3 of the VH amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSS (SEQ ID NO:54), wherein, optionally, the CDRs are defined according to IMGT; and (ii) a light chain comprising, in a VL, CDRL1, CDRL2, and CDRL3 of the VL amino acid sequence

(SEQ ID NO.: 8)
EIVMTQSPATLSLSSGERATLSCRASRSVSSNLAWYQQKPGQAPRLLIY
DASTRATGFSARFAGSGSGTEFTLTISSLQSEDSAIYYCQQYNNWPPWT
FGQGTKVEIK.

wherein, optionally, the CDRs are defined according to IMGT. In certain further embodiments, the antibody or antigen-binding fragment is an (e.g. human) IgG1 isotype and optionally comprises M428L and N434S mutations. In some embodiments, the light chain is an IgG1 kappa light chain.

In certain embodiments, an antibody or antigen-binding fragment is provided that comprises: (i) two heavy chains each comprising, in a VH, CDRH1, CDRH2, and CDRH3 of the VH amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSS (SEQ ID NO:54) wherein, optionally, the CDRs are defined according to IMGT; and (ii) two light chains each comprising, in a VL, CDRL1, CDRL2, and CDRL3 of the VL amino acid sequence

(SEQ ID NO.: 8)
EIVMTQSPATLSLSSGERATLSCRASRSVSSNLAWYQQKPGQAPRLLIY
DASTRATGFSARFAGSGSGTEFTLTISSLQSEDSAIYYCQQYNNWPPWT
FGQGTKVEIK.

wherein, optionally, the CDRs are defined according to IMGT. In certain further embodiments, the antibody or antigen-binding fragment is an (e.g. human) IgG1 isotype and optionally comprises M428L and N434S mutations. In some embodiments, the light chains are each an IgG1 kappa light chain.

In some embodiments, an antibody or antigen-binding fragment is provided that comprises (i) a VH comprising or consisting of the amino acid sequence of any one of SEQ ID NOs:2, 45, 46, 48, 50, 52, 54, 56, 58, 60, 63, and 65, and (ii) a VL comprising or consisting of the VL amino acid sequence of SEQ ID NO:37.

In some embodiments, an antibody or antigen-binding fragment is provided that comprises (i) a VH comprising or consisting of the amino acid sequence of any one of SEQ ID NOs:2, 45, 46, 48, 50, 52, 54, 56, 58, 60, 63, and 65, and (ii) a VL comprising or consisting of the VL amino acid sequence of SEQ ID NO:66.

In some embodiments, an antibody or antigen-binding fragment is provided that comprises (i) a VH comprising or consisting of the amino acid sequence of any one of SEQ ID NOs:2, 45, 46, 48, 50, 52, 54, 56, 58, 60, 63, and 65, and (ii) a VL comprising or consisting of the VL amino acid sequence of SEQ ID NO:68.

TABLE A
provides VH and VL amino acid SEQ ID NOs. of certain
FNI9 variant antibodies of the present disclosure.

| Variant | VH amino acid | VL amino acid |
|---|---|---|
| Antibody | SEQ ID NO.: | SEQ ID NO.: |
| FNI9-v13.8 | 65 | 68 |
| FNI9-v4.1 | 46 | 8 |
| FNI9-v5.1 | 48 | 8 |
| FNI9-v6.1 | 50 | 8 |
| FNI9-v7.1 | 52 | 8 |
| FNI9-v8.1 | 54 | 8 |
| FNI9-v9.1 | 56 | 8 |
| FNI9-v10.1 | 58 | 8 |
| FNI9-v11.1 | 60 | 8 |
| FNI9-v12.1 | 63 | 8 |
| FNI9-v4.7 | 46 | 66 |
| FNI9-v5.7 | 48 | 66 |
| FNI9-v6.7 | 50 | 66 |
| FNI9-v7.7 | 52 | 66 |
| FNI9-v8.7 | 54 | 66 |
| FNI9-v9.7 | 56 | 66 |
| FNI9-v10.7 | 58 | 66 |
| FNI9-v11.7 | 60 | 66 |
| FNI9-v12.7 | 63 | 66 |

In some embodiments, the influenza comprises an influenza A virus, an influenza B virus, or both.

In certain embodiments, an antibody or antigen-binding fragment of the present disclosure is monospecific (e.g., binds to a single epitope) or is multispecific (e.g., binds to multiple epitopes and/or target molecules). Antibodies and antigen binding fragments may be constructed in various formats. Exemplary antibody formats disclosed in Spiess et al., Mol. Immunol. 67(2):95 (2015), and in Brinkmann and Kontermann, mAbs 9(2): 182-212 (2017), which formats and methods of making the same are incorporated herein by reference and include, for example, Bispecific T cell Engagers (BITES), DARTs, Knobs-Into-Holes (KIH) assemblies, scFv-CH3-KIH assemblies, KIH Common Light-Chain antibodies, TandAbs, Triple Bodies, TriBi Minibodies, Fab-scFv, scFv-CH-CL-scFv, F(ab′)2-scFv2, tetravalent Hcabs, Intrabodies, CrossMabs, Dual Action Fabs (DAFs) (two-in-one or four-in-one), DutaMabs, DT-IgG, Charge Pairs, Fab-arm Exchange, SEEDbodies, Triomabs, LUZ-Y assemblies, Fcabs, rc-bodies, orthogonal Fabs, DVD-Igs (e.g., U.S. Pat. No. 8,258,268, which formats are incorporated herein by reference in their entirety), IgG(H)-scFv, scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, Zybody, and DVI-IgG (four-in-one), as well as so-called FIT-Ig (e.g., PCT Publication No. WO 2015/103072, which formats are incorporated herein by reference in their entirety), so-called WuxiBody formats (e.g., PCT Publication No. WO 2019/057122, which formats are incorporated herein by reference in their entirety), and so-called In-Elbow-Insert Ig formats (IEI-Ig; e.g., PCT Publication Nos. WO 2019/024979 and WO 2019/025391, which formats are incorporated herein by reference in their entirety).

In certain embodiments, the antibody or antigen-binding fragment comprises two or more of VH domains, two or more VL domains, or both (i.e., two or more VH domains and two or more VL domains). In particular embodiments, an antigen-binding fragment comprises the format (N-terminal to C-terminal direction) VH-linker-VL-linker-VH-linker-VL, wherein the two VH sequences can be the same or different and the two VL sequences can be the same or different. Such linked scFvs can include any combination of VH and VL domains arranged to bind to a given target, and in formats comprising two or more VH and/or two or more VL, one, two, or more different epitopes or antigens may be bound. It will be appreciated that formats incorporating multiple antigen-binding domains may include VH and/or VL sequences in any combination or orientation. For example, the antigen-binding fragment can comprise the format VL-linker-VH-linker-VL-linker-VH, VH-linker-VL-linker-VL-linker-VH, or VL-linker-VH-linker-VH-linker-VL.

Monospecific or multispecific antibodies or antigen-binding fragments of the present disclosure constructed comprise any combination of the VH and VL sequences and/or any combination of the CDRH1, CDRH2, CDRH3, CDRL1, CDRL2, and CDRL3 sequences disclosed herein. A bispecific or multispecific antibody or antigen-binding fragment may, in some embodiments, comprise one, two, or more antigen-binding domains (e.g., a VH and a VL) of the instant disclosure. Two or more binding domains may be present that bind to the same or a different NA epitope, and a bispecific or multispecific antibody or antigen-binding fragment as provided herein can, in some embodiments, comprise a further NA-specific binding domain, and/or can comprise a binding domain that binds to a different antigen or pathogen altogether.

In any of the presently disclosed embodiments, the antibody or antigen-binding fragment can be multispecific; e.g., bispecific, trispecific, or the like.

In certain embodiments, the antibody or antigen-binding fragment comprises a Fc polypeptide, or a fragment thereof. The “Fc” fragment or Fc polypeptide comprises the carboxy-terminal portions (i.e., the CH2 and CH3 domains of IgG) of both antibody H chains held together by disulfides. An Fc may comprise a dimer comprised of two Fc polypeptides (i.e., two CH2-CH3 polypeptides). Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation. As discussed herein, modifications (e.g., amino acid substitutions) may be made to an Fc domain in order to modify (e.g., improve, reduce, or ablate) one or more functionality of an Fc-containing polypeptide (e.g., an antibody of the present disclosure). Such functions include, for example, Fc receptor (FcR) binding, antibody half-life modulation (e.g., by binding to FcRn), ADCC function, protein A binding, protein G binding, and complement binding. Amino acid modifications that modify (e.g., improve, reduce, or ablate) Fc functionalities include, for example, the T250Q/M428L, M252Y/S254T/T256E, H433K/N434F, M428L/N434S, M428L/434A, E233P/L234V/L235A/G236+A327G/A330S/P331S, E333A, S239D/A330L/I332E, P257I/Q311, K326W/E333S, S239D/I332E/G236A, N297Q, K322A, S228P, L235E+E318A/K320A/K322A, L234A/L235A (also referred to herein as “LALA”), and L234A/L235A/P329G mutations, certain of which mutations are summarized and annotated in “Engineered Fe Regions”, published by InvivoGen (2011) and available online at invivogen.com/PDF/review/review-Engineered-Fc-Regions-invivogen.pdf?utm_source=review&utm_medium=pdf&utm_campaign=review&utm_content=Engineered-Fc-Regions, and are incorporated herein by reference.

For example, to activate the complement cascade, the C1q protein complex can bind to at least two molecules of IgG1 or one molecule of IgM when the immunoglobulin molecule(s) is attached to the antigenic target (Ward, E. S., and Ghetie, V., Ther. Immunol. 2 (1995) 77-94). Burton, D. R., described (Mol. Immunol. 22 (1985) 161-206) that the heavy chain region comprising amino acid residues 318 to 337 is involved in complement fixation. Duncan, A. R., and Winter, G. (Nature 332 (1988) 738-740), using site directed mutagenesis, reported that Glu318, Lys320 and Lys322 form the binding site to C1q. The role of Glu318, Lys320 and Lys 322 residues in the binding of C1q was confirmed by the ability of a short synthetic peptide containing these residues to inhibit complement mediated lysis.

For example, FcR binding can be mediated by the interaction of the Fc moiety (of an antibody) with Fc receptors (FcRs), which are specialized cell surface receptors on cells including hematopoietic cells. Fc receptors belong to the immunoglobulin superfamily, and shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC; Van de Winkel, J. G., and Anderson, C. L., J Leukoc. Biol. 49 (1991) 511-524). FcRs are defined by their specificity for immunoglobulin classes; Fc receptors for IgG antibodies are referred to as FcγR, for IgE as FcεR, for IgA as FcαR and so on and neonatal Fc receptors are referred to as FcRn. Fc receptor binding is described for example in Ravetch, J. V., and Kinet, J. P., Annu. Rev. Immunol. 9 (1991) 457-492; Capel, P. J., et al., Immunomethods 4 (1994) 25-34; de Haas, M., et al., J Lab. Clin. Med. 126 (1995) 330-341; and Gessner, J. E., et al., Ann. Hematol. 76 (1998) 231-248.

Cross-linking of receptors by the Fc domain of native IgG antibodies (FcγR) triggers a wide variety of effector functions including phagocytosis, antibody-dependent cellular cytotoxicity, and release of inflammatory mediators, as well as immune complex clearance and regulation of antibody production. Fc moieties providing cross-linking of receptors (e.g., FcγR) are contemplated herein. In humans, three classes of FcγR have been characterized to-date, which are: (i) FcγRI (CD64), which binds monomeric IgG with high affinity and is expressed on macrophages, monocytes, neutrophils and eosinophils; (ii) FcγRII (CD32), which binds complexed IgG with medium to low affinity, is widely expressed, in particular on leukocytes, is believed to be a central player in antibody-mediated immunity, and which can be divided into FcγRIIA, FcγRIIB and FcγRIIC, which perform different functions in the immune system, but bind with similar low affinity to the IgG-Fc, and the ectodomains of these receptors are highly homolougous; and (iii) FcγRIII (CD16), which binds IgG with medium to low affinity and has been found in two forms: FcγRIIIA, which has been found on NK cells, macrophages, eosinophils, and some monocytes and T cells, and is believed to mediate ADCC; and FcγRIIIB, which is highly expressed on neutrophils.

FcγRIIA is found on many cells involved in killing (e.g. macrophages, monocytes, neutrophils) and seems able to activate the killing process. FcγRIIB seems to play a role in inhibitory processes and is found on B-cells, macrophages and on mast cells and eosinophils. Importantly, it has been shown that 75% of all FcγRIIB is found in the liver (Ganesan, L. P. et al., 2012: “FcγRIIb on liver sinusoidal endothelium clears small immune complexes,” Journal of Immunology 189: 4981-4988). FcγRIIB is abundantly expressed on Liver Sinusoidal Endothelium, called LSEC, and in Kupffer cells in the liver and LSEC are the major site of small immune complexes clearance (Ganesan, L. P. et al., 2012: FcγRIIb on liver sinusoidal endothelium clears small immune complexes. Journal of Immunology 189: 4981-4988).

In some embodiments, the antibodies disclosed herein and the antigen-binding fragments thereof comprise an Fc polypeptide or fragment thereof for binding to FcγRIIb, in particular an Fc region, such as, for example IgG-type antibodies. Moreover, it is possible to engineer the Fc moiety to enhance FcγRIIB binding by introducing the mutations S267E and L328F as described by Chu, S. Y. et al., 2008: Inhibition of B cell receptor-mediated activation of primary human B cells by coengagement of CD19 and FcγRIIb with Fc-engineered antibodies. Molecular Immunology 45, 3926-3933. Thereby, the clearance of immune complexes can be enhanced (Chu, S., et al., 2014: Accelerated Clearance of IgE In Chimpanzees Is Mediated By Xmab7195, An Fc-Engineered Antibody With Enhanced Affinity For Inhibitory Receptor FcγRIIb. Am J Respir Crit, American Thoracic Society International Conference Abstracts). In some embodiments, the antibodies of the present disclosure, or the antigen binding fragments thereof, comprise an engineered Fc moiety with the mutations S267E and L328F, in particular as described by Chu, S. Y. et al., 2008: Inhibition of b cell receptor-mediated activation of primary human B cells by coengagement of CD19 and FcγRIIb with Fc-engineered antibodies. Molecular Immunology 45, 3926-3933.

On B cells, FcγRIIB may function to suppress further immunoglobulin production and isotype switching to, for example, the IgE class. On macrophages, FcγRIIB is thought to inhibit phagocytosis as mediated through FcγRIIA. On eosinophils and mast cells, the B form may help to suppress activation of these cells through IgE binding to its separate receptor.

Regarding FcγRI binding, modification in native IgG of at least one of E233-G236, P238, D265, N297, A327 and P329 reduces binding to FcγRI. IgG2 residues at positions 233-236, substituted into corresponding positions IgG1 and IgG4, reduces binding of IgG1 and IgG4 to FcγRI by 10³-fold and eliminated the human monocyte response to antibody-sensitized red blood cells (Armour, K. L., et al. Eur. J Immunol. 29 (1999) 2613-2624).

Regarding FcγRII binding, reduced binding for FcγRIIA is found, e.g., for IgG mutation of at least one of E233-G236, P238, D265, N297, A327, P329, D270, Q295, A327, R292 and K414.

Two allelic forms of human FcγRIIA are the “H131” variant, which binds to IgG1 Fc with higher affinity, and the “R131” variant, which binds to IgG1 Fc with low affinity. See, e.g., Bruhns et al., Blood 113:3716-3725 (2009).

Regarding FcγRIII binding, reduced binding to FcγRIIIA is found, e.g., for mutation of at least one of E233-G236, P238, D265, N297, A327, P329, D270, Q295, A327, S239, E269, E293, Y296, V303, A327, K338 and D376. Mapping of the binding sites on human IgG1 for Fc receptors, the above-mentioned mutation sites, and methods for measuring binding to FcγRI and FcγRIIA, are described in Shields, R. L., et al., J Biol. Chem. 276 (2001) 6591-6604.

Two allelic forms of human FcγRIIIA are the “F158” variant, which binds to IgG1 Fc with lower affinity, and the “V158” variant, which binds to IgG1 Fc with higher affinity. See, e.g., Bruhns et al., Blood 113:3716-3725 (2009).

Regarding binding to FcγRII, two regions of native IgG Fc appear to be involved in interactions between FcγRIIs and IgGs, namely (i) the lower hinge site of IgG Fc, in particular amino acid residues L, L, G, G (234-237, EU numbering), and (ii) the adjacent region of the CH2 domain of IgG Fc, in particular a loop and strands in the upper CH2 domain adjacent to the lower hinge region, e.g. in a region of P331 (Wines, B. D., et al., J. Immunol. 2000; 164: 5313-5318). Moreover, FcγRI appears to bind to the same site on IgG Fc, whereas FcRn and Protein A bind to a different site on IgG Fc, which appears to be at the CH2-CH3 interface (Wines, B. D., et al., J. Immunol. 2000; 164: 5313-5318).

Also contemplated are mutations that increase binding affinity of an Fc polypeptide or fragment thereof of the present disclosure to a (i.e., one or more) Fcγ receptor (e.g., as compared to a reference Fc polypeptide or fragment thereof or containing the same that does not comprise the mutation(s)). See, e.g., Delillo and Ravetch, Cell 161(5):1035-1045 (2015) and Ahmed et al., J. Struc. Biol. 194(1):78 (2016), the Fc mutations and techniques of which are incorporated herein by reference.

In any of the herein disclosed embodiments, an antibody or antigen-binding fragment can comprise a Fc polypeptide or fragment thereof comprising a mutation selected from G236A; S239D; A330L; and I332E; or a combination comprising any two or more of the same; e.g., S239D/I332E; S239D/A330L/I332E; G236A/S239D/I332E; G236A/A330L/I332E (also referred to herein as “GAALIE”); or G236A/S239D/A330L/I332E. In some embodiments, the Fc polypeptide or fragment thereof does not comprise S239D. In some embodiments, the Fc polypeptide or fragment thereof comprises S at position 239 (EU numbering).

In certain embodiments, the Fc polypeptide or fragment thereof may comprise or consist of at least a portion of an Fc polypeptide or fragment thereof that is involved in FcRn binding. In certain embodiments, the Fc polypeptide or fragment thereof comprises one or more amino acid modifications that improve binding affinity for (e.g., enhance binding to) FcRn (e.g., at a pH of about 6.0) and, in some embodiments, thereby extend in vivo half-life of a molecule comprising the Fc polypeptide or fragment thereof (e.g., as compared to a reference Fc polypeptide or fragment thereof or antibody that is otherwise the same but does not comprise the modification(s)). In certain embodiments, the Fc polypeptide or fragment thereof comprises or is derived from a IgG Fc and a half-life-extending mutation comprises any one or more of: M428L; N434S; N434H; N434A; N434S; M252Y; S254T; T256E; T250Q; P257I Q311I; D376V; T307A; E380A (EU numbering). In certain embodiments, a half-life-extending mutation comprises M428L/N434S (also referred to herein as “MLNS”, “LS”, “_LS”, and “-LS”). In certain embodiments, a half-life-extending mutation comprises M252Y/S254T/T256E. In certain embodiments, a half-life-extending mutation comprises T250Q/M428L. In certain embodiments, a half-life-extending mutation comprises P257I/Q311I. In certain embodiments, a half-life-extending mutation comprises P257I/N434H. In certain embodiments, a half-life-extending mutation comprises D376V/N434H. In certain embodiments, a half-life-extending mutation comprises T307A/E380A/N434A. In certain embodiments, a half-life-extending mutation comprises M428L/N434A.

In some embodiments, an antibody or antigen-binding fragment includes a Fc moiety that comprises the substitution mutations M428L/N434S. In some embodiments, an antibody or antigen-binding fragment includes a Fc moiety that comprises the substitution mutations M428L/N434A. In some embodiments, an antibody or antigen-binding fragment includes a Fc polypeptide or fragment thereof that comprises the substitution mutations G236A/A330L/I332E.

In certain embodiments, an antibody or antigen-binding fragment includes a (e.g., IgG) Fc moiety that comprises a G236A mutation, an A330L mutation, and a I332E mutation (GAALIE), and does not comprise a S239D mutation (e.g., comprises a native S at position 239). In particular embodiments, an antibody or antigen-binding fragment includes an Fc polypeptide or fragment thereof that comprises the substitution mutations: M428L/N434S and G236A/A330L/I332E, and optionally does not comprise S239D (e.g., comprises S at 239). In certain embodiments, an antibody or antigen-binding fragment includes a Fc polypeptide or fragment thereof that comprises the substitution mutations: M428L/N434S and G236A/S239D/A330L/I332E.

In certain embodiments, the antibody or antigen-binding fragment comprises a mutation that alters glycosylation, wherein the mutation that alters glycosylation comprises N297A, N297Q, or N297G, and/or the antibody or antigen-binding fragment is partially or fully aglycosylated and/or is partially or fully afucosylated. Host cell lines and methods of making partially or fully aglycosylated or partially or fully afucosylated antibodies and antigen-binding fragments are known (see, e.g., PCT Publication No. WO 2016/181357; Suzuki et al. Clin. Cancer Res. 13(6):1875-82 (2007); Huang et al. mAbs 6:1-12 (2018)).

In certain embodiments, an antibody or antigen-binding fragment comprises a heavy chain that comprises one or more mutations in the hinge, CH2, and/or CH3 (or in the Fc), wherein the antibody or antigen-binding fragment has one or more improved characteristics over, for example, the antibody or antigen-binding fragment comprising reference wild-type Fc polypeptide and/or comprising a known variant Fc polypeptide). Presently disclosed Fc variants possess, for example: increased binding to one or more human FcγRA (e.g., a FcγRIIA and/or a FcγRIIIA; decreased/reduced binding to a human FcγRIIB; increased binding to one or more human FcγRA as compared to binding to a human FcγRIIB; increased thermostability as compared to known Fc polypeptides; increased binding to human C1q; increased human FcγRIIIA signaling in a host cell expressing the FcγRIIIA, increased human FcγRIIIA signaling in a host cell expressing the FcγRIIA, decreased human FcγRIIB signaling in a host cell expressing the FcγRIIB, a relative increase in binding to FcγRA as compared to FcγRIIB, improved qualities for production as compared to known Fc polypeptides; and combinations of such features.

In certain embodiments, antibodies comprising a variant Fc polypeptide of the present disclosure provide surprising advantages, such as any one or more of the following: increased binding affinity (e.g. as determined by surface plasmon resonance, e.g. using a Biacore instrument and/or as determined by a electrochemiluminescence assay, such as a meso scale discovery (MSD) assay) for and/or inducing increased signaling (e.g. as determined using (1) an Fc variant antibody (2) antigen-expressing target cells and (3) reporter cells expressing one or more human FcγRA, optionally driving expression of a reporter gene such as, for example, GFP or luciferase) by one or more human FcγRA, as compared to the antibody comprising a reference Fc polypeptide not comprising the mutation(s) and/or fucosylation state; decreased binding affinity for and/or inducing decreased signaling of human FcγRIIB, as compared to the antibody comprising a reference Fc polypeptide not comprising the mutation(s) and/or fucosylation state; a unique and optionally improved binding profile across human FcγRIIA-H, human FcγRIIA-R, human FcγRIIB, human FcγRIIIA-F, and human FcγRIIIA-V, wherein improved binding comprises an overall increase in binding to and/or activation of FcγRA signaling relative to binding to and/or activation of inhibitory FcγR signaling, as compared to the antibody comprising a reference Fc polypeptide not comprising the mutation(s) and/or fucosylation state; increased binding affinity for human C1q, as compared to the antibody comprising a reference Fc polypeptide not comprising the mutation(s) and/or fucosylation state; no detrimental effect or no substantial detrimental effect on thermal stability, a reduced negative effect on thermal stability as compared to a variant Fc polypeptide or fragment thereof not comprising the mutation(s) and/or fucosylation state (e.g., a human IgG1 Fc comprising the mutations G236A, A330L, and I332E (e.g. having a smaller decreasing effect, or no decreasing effect, on melting temperature as compared to the antibody comprising a human IgG1 Fc comprising the mutations G236A, A330L, and I332E), or having a higher melting temperature than the antibody comprising a human IgG1 Fc comprising the mutations G236A, A330L, and I332E)); increasing specific lysis (e.g. via ADCC) by natural killer cells and/or PBMCs (e.g. expressing F158/V158 or V158/V158 FcγRIIIA) against antigen-expressing target cells, as compared to the antibody comprising a reference Fc polypeptide not comprising the mutation(s) and/or fucosylation state (e.g. the antibody comprising a human IgG1 Fc comprising the mutations G236A, A330L, and I332E); increasing ADCP by monocytes (e.g. CD14+ monocytes, optionally expressing F158/V158 FcγRIIA and R131/H131 FcγRIIA or F158/F158 FcγRIIA and R131/H131 FcγRIIA) against antigen-expressing target cells, as compared to the antibody comprising a reference Fc polypeptide not comprising the mutation(s) and/or fucosylation state; increasing the percentage of CD83+ cells (e.g. moDCs) and/or increasing expression of CD83 by moDCs in a sample when provided in combination with the antigen, as compared to the antibody comprising a reference Fc polypeptide not comprising the mutation(s) and/or fucosylation state, when provided in combination with the antigen; increasing production of one or more cytokine (optionally selected from the group consisting of IL-10, IFN-γ, IL-6, and TNF-α) by moDCs in a sample when provided in combination with the antigen, as compared to the antibody comprising a reference Fc polypeptide not comprising the mutation(s) and/or fucosylation state, when provided in combination with the antigen; and/or increasing the ability of moDCs to stimulate antigen-specific CD4+ T cells when provided to the moDCs in combination with the antigen, as compared to the antibody comprising a reference Fc polypeptide not comprising the mutation(s) and/or fucosylation state, when provided to the moDCs in combination with the antigen, wherein, optionally, (1) the moDCs and the CD4+ T cells are from the same (optionally antigen-vaccinated) subject and/or (2) stimulation of antigen-specific CD4+ T cells is determined by an increase in CD25 expression and/or an increase in proliferation (e.g. as determined by a reduction in CFSE staining over time) and/or an increase in expression of CD69 and/or an increase in expression of NFAT and/or an increase in expression of CD44, by the antigen-specific CD4+ T cells.

In some embodiments, an engineered Fc or Fc fragment of the present disclosure (or a polypeptide comprising the same) comprises two or more substitution mutations as compared to a reference wild-type Fc or Fc fragment, and the combined effect of the two or more substitutions is different than, and is optionally greater than, would be expected based on the effects of the individual component substitution mutations and/or based on the effects of a subset of the two or more substitution mutations. In other words, in some embodiments, combination mutations comprise a non-additive or synergistic effect with reference to the individual component mutations and/or to a subset thereof.

In some embodiments, presently disclosed antibodies or antigen-binding fragments comprising an Fc variant possess characteristics such as effector functions, ability to bind human C1q, ability to induce FcγRA-mediated cell signaling, ability to bind to human FcRn, ability to promote ADCP, ability to promote ADCC, ability to promote activation of CD4+ T cells, and the like.

Presently disclosed polypeptides include those that comprise a variant of: an IgG Fc polypeptide or a fragment thereof, wherein the variant comprises one or more modifications as compared to the IgG Fc polypeptide or fragment thereof. It will be understood that, unless stated otherwise, a “reference” polypeptide or antibody (e.g., reference IgG Fc polypeptide or fragment thereof, reference antibody, reference CH2 polypeptide, reference IgG hinge-CH2, reference IgG hinge-Fc polypeptide, reference CH3 polypeptide) is preferably identical to the recited molecule (e.g., variant of an Fc polypeptide or fragment thereof; polypeptide comprising such a variant; antibody comprising a variant of an Fc polypeptide) except for the recited difference or differences.

For example, it will be understood that for a variant IgG1 Fc polypeptide that comprises an alanine (A) amino acid at EU position 236, a reference Fc polypeptide includes an IgG1 Fc polypeptide that is otherwise identical to the variant except that a native glycine (G) amino acid is found at EU position 236. As another example, for a variant of an Fc polypeptide fragment (e.g., containing a CH2 and a portion of a CH3), a reference Fc polypeptide fragment is preferably of an identical length to the variant and preferably differs from the variant only by the recited features (e.g., amino acid mutation or mutations present in the variant). In some embodiments, a reference Fc polypeptide, Fc polypeptide fragment, or antibody comprises a wild-type amino acid sequence (e.g., wild-type human IgG1). Excepting the recited differences present in the variant, a reference Fc polypeptide, Fc polypeptide fragment, or antibody will be of the same isotype, and, preferably, of the same allotype, as the variant. In the case of a reference antibody, the Fabs or other antigen-binding domains will preferably be identical to those present in the specified antibody comprising a variant Fc polypeptide or fragment thereof.

In some embodiments, variants of IgG Fc polypeptides or fragments thereof include one or more amino acid substitution as compared to a reference (e.g. wild-type) IgG Fc polypeptide or fragment thereof. Herein, the position of an amino acid in a variant IgG Fc polypeptide or fragment may be described by referencing the “EU position”; it will be understood that “the EU position” follows the EU numbering system as set forth in Kabat. By way of illustration, it will be understood that in the example of a human IgG1 CH1-CH3 amino acid sequence provided below, the first amino acid (A) corresponds to EU position 118, and the last amino acid (K) corresponds to EU position 447:

ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKKVEP KSCDKTHTCP PCPAPELLGG
PSVFLFPPKP KDTLMISRTP EVTCVVVDVS HEDPEVKFNW
YVDGVEVHNA KTKPREEQYN STYRVVSVLT VLHQDWLNGK
EYKCKVSNKA LPAPIEKTIS KAKGQPREPQ VYTLPPSRDE
LTKNQVSLTC LVKGFYPSDI AVEWESNGQP ENNYKTTPPV
LDSDGSFFLY SKLTVDKSRW QQGNVFSCSV MHEALHNHYT
QKSLSLSPGK

Accordingly, it will be understood that unless otherwise indicated, the position of a recited amino acid(s) follows EU numbering for human IgG1 even if a complete antibody heavy chain, complete CH1-CH3, complete CH2-CH3, or the like is not present or is not explicitly recited. In other words, for example, if only a hinge-CH2 is described and a CH3 and/or CH1 may not be present, the position of the amino acids in the hinge-CH2 is described with reference to EU numbering, unless stated otherwise. Correspondence between EU numbering, Kabat numbering, IMGT exon numbering, and IMGT unique numbering for immunoglobulin G heavy chain constant domain is known in the art and is shown, for example, in the IMGT Scientific chart (www.imgt.org/IMGTScientificChart/Numbering/Hu_IGHGnber.html; created May 17, 2001, accessed May 23, 2021, last updated Jan. 20, 2020).

Certain embodiments of Fc variants of the present disclosure (fucosylated, unless otherwise indicated) and non-limiting properties of the same are summarized in Table B.

TABLE B
Certain Fc Variants and Properties Thereof

| Variant (substitution | Certain properties of the indicated |
|---|---|
| mutation(s) vs. wild- | variant(s), as compared to |
| type human IgG1 Fc) | fucosylated wild-type human IgG1 |
| G236A_L328V_Q295E | Increased binding to human FcγRIIa (H131 allele and |
| G236A_P230A_Q295E | R131 allele); comparable or decreased binding to human |
| G236A_R292P_I377N | FcγRIIb (e.g. by MSD assay and/or surface plasmon |
| G236A_K334A_Q295E | resonance); increased ratio of: binding to human FcγRIIa |
| G236S_R292P_Y300L | (H131 allele or R131 allele) versus binding to human |
| | FcγRIIb; comparable binding to human FcRn; comparable |
| | production titer; increased signaling in a host cell via |
| | FcγRIIa and/or decreased signaling in a host cell via |
| | FcγRIIb; Tm within 12° C. or less of wild-type; |
| | G236S_R292P_Y300L has improved binding to C1q |
| G236A_Y300L | Increased binding to human FcγRIIa (H131 (over 18-fold) |
| | and R131 (over 4-fold)); similar binding to human FcγRIIb |
| | or reduced binding to human FcγRIIb (e.g. as measured by |
| | surface plasmon resonance); increased ratio of: binding to |
| | human FcγRIIa (H131 or R131) versus binding to human |
| | FcγRIIb; comparable binding to human FcRn; comparable |
| | production titer; increased signaling in a host cell via |
| | FcγRIIa and/or decreased signaling in a host cell via |
| | FcγRIIb; Tm within 4.5° C. of wild-type |
| G236A_R292P_Y300L | Increased binding to human FcγRIIa (H131 (over 14-fold) |
| | and R131 (over 2.7-fold)); similar binding to human |
| | FcγRIIb; increased ratio of: binding to human FcγRIIa |
| | (H131 or R131) versus binding to human FcγRIIb; |
| | increased binding to human FcγRIIIa (V158 allele and |
| | F158 allele); comparable binding to human FcRn; |
| | comparable production titer; increased signaling in a host |
| | cell via FcγRIIa and/or FcγRIIIa, and/or decreased |
| | signaling in a host cell via FcγRIIb; increased signaling in |
| | a host cell via FcγRIIa and/or decreased signaling in a host |
| | cell via FcγRIIb; Tm within 4° C. of wild-type; comparable |
| | binding to human C1q |
| G236S_G420V_G446E_L309T | Increased binding to human FcγRIIa; decreased binding to |
| G236A_R292P | human FcγRIIb (less than 0.5-fold); increased ratio of: |
| | binding to human FcγRIIa (H131 or R131) versus binding |
| | to human FcγRIIb; comparable binding to human FcRn; |
| | comparable production titer; increased signaling in a host |
| | cell via FcγRIIa and/or FcγRIIIa, and/or decreased |
| | signaling in a host cell via FcγRIIb; Tm within 4° C. |
| | or less of wild-type |
| R292P_Y300L | Increased binding to human FcγRIIIa (V158 and F158); |
| | increased binding to human C1q; Tm within 4° C. of wild- |
| | type |
| Y300L | Increased binding to human C1q |
| E345K_G236S_L235Y_S267E | |
| E272R_L309T_S219Y_S267E | |
| G236Y | |
| G236W | |
| F243L_G446E_P396L_S267E | |
| G236A (afucosylated) | Increased binding to human FcγRIIa (H131) and mouse |
| | FcγRIIa (R131), decreased binding human FcγRIIb, |
| | increased binding to human FcγRIIIa (V158) and mouse |
| | FcγRIIIa (F158), increased binding to human FcγRIIIb, |
| | somewhat decreased binding to human FcRn, Tm within 0.15° C. |
| | of wild-type or within 0.9° C. of wild-type or within |
| | 0.8° C. of wild-type or within 0.7° C. of wild-type |
| S239D_H268E_G236A | Increased binding to and signaling via all human FcγRs |
| | tested: FcγRIIA (H131); FcγRIIA (R131); FcγRIIB; |
| | FcγRIIIA (V158); FcγRIIIA (F158); FcγRIIIB; |
| | additionally, when anti-HBV antibody bearing |
| | S239D_H268E_G236A_M428L_N434S was combined |
| | with hBsAg, the immune complexes formed thereby were |
| | incubated with MoDCs; subsequent incubation of the |
| | MoDCs with donor CD4+ T cells resulted in an increased |
| | percentage of NFAT+ CD69+ CD3+ CD4+ T cells as |
| | compared to antibodies bearing M428L_N434S only. |

Additional features of disclosed Fc variant-containing antibodies are shown in the present Examples and Figures, and described herein.

It will be understood that two or more amino acid substitutions present in a variant can be expressed in a variety of ways, for example, as G236A_Y300L, or as G236ANY300L. Moreover, a mutation or combination mutation may be referenced using a short form including the original amino acid(s) and the amino acid(s) resulting from the substitution(s). For example, G236A may be described as “GA” or “236A”; G236A_Y300L may be described as “GAYL”; G236A_L328V_Q295E may be described as “GALVQE”; G236A_R292P_Y300L may be described as “GARPYL”, G236A_R292P_I377N may be described as “GARPIN”, or the like.

In any of the presently disclosed embodiments, a variant of an Fc polypeptide or fragment thereof can be derived from or comprise a human Fc polypeptide or fragment thereof, and/or can be derived from or comprise a human IgG1, a human IgG2, a human IgG3, or a human IgG4 isotype. In this context, the expression “derived from” means that the variant is the same as the referenced polypeptide or isotype, with the exception of the specified modification(s) (e.g., amino acid substitution(s)). By way of example, a variant Fc polypeptide which comprises a wild-type human IgG1 Fc amino acid sequence with the exception of the amino acid substitution mutations G236A_L328V_Q295E (and, optionally, other amino acid substitutions) can be said to be “derived from” wild-type human IgG1 Fc. In any of the presently disclosed embodiments, a polypeptide, CH2, Fc, Fc fragment, or antibody may comprise human Ig sequence, such as human IgG1 sequence. In some embodiments, the polypeptide, CH2, Fc, Fc fragment, or antibody can comprise a native or wild-type human Ig sequence with the exception of the described mutation(s), or can comprise a human Ig (e.g. IgG) sequence that contains one or more additional mutations.

An antibody or antigen-binding fragment, may be of any allotype or combination of allotypes. “Allotype” refers to the allelic variation found among the IgG subclasses. For example, an allotype may comprise G1m1 (or G1m(a)), G1m2 (or G1m(x)), G1m3 (or G1m(f)), G1m17 (or Gm(z))m), G1m27, and/or G1m28 (G1m27 and G1m28 have been described as “alloallotypes”).

The G1m3 and G1m17 allotypes are located at the same position in the CH1 domain (position 214 according to EU numbering). G1m3 comprises R214 (EU), while G1m17 comprises K214 (EU). The G1 ml allotype is located in the CH3 domain (at positions 356 and 358 (EU)) and refers to the replacements E356D and M358L. The G1m2 allotype refers to a replacement of the alanine in position 431 (EU) by a glycine. G1m allotypes, alloallotypes, and features thereof are known in the art and described at, for example, www.imgt.org/IMGTrepertoire/Proteins/allotypes/human/IGH/IGHC/G1m_allotypes.html and Lefranc, M.-P. and Lefranc, G. Human Gm, Km and Am allotypes and their molecular characterization: a remarkable demonstration of polymorphism In: B. Tait, F. Christiansen (Eds.), Immunogenetics, chap. 34, Humana Press, Springer, New York, USA. Methods Mol. Biol. 2012; 882, 635-680. PMID: 22665258, LIGM: 406, the contents and allotypes and allotype information of which are incorporated herein by reference.

The G1 ml allotype may be combined, for example, with the G1m3, G1m17, G1m27, G1m2, and/or G1m28 allotype. In some embodiments, an allotype is G1m3 with no G1 ml (G1m3,−1). In some embodiments, an allotype is G1m17,1 allotype. In some embodiments, an allotype is G1m3,1. In some embodiments, an allotype is G1m17 with no G1 ml (G1m17,−1). Optionally, these allotypes may be combined (or not combined) with the G1m2, G1m27 or G1m28 allotype. For example, an allotype may be G1m17,1,2.

In some embodiments, an antibody or antigen-binding fragment of the present disclosure comprises a G1m3 allotype or a G1m3,1 allotype. In some embodiments, an antibody or antigen-binding fragment of the present disclosure comprises a G1m3 allotype and comprises M428L and N434S or M428L and N434A mutations or any other mutation(s) that enhance binding to a human FcRn, such as those described herein. In some embodiments, an antibody or antigen-binding fragment of the present disclosure comprises a G1m3,1 allotype and comprises M428L and N434S or M428L and N434A mutations or any other mutation(s) that enhance binding to a human FcRn, such as those described herein. In some embodiments, an antibody or antigen-binding fragment of the present disclosure comprises a G1m17, 1 allotype. In some embodiments, an antibody or antigen-binding fragment of the present disclosure comprises a G1m17, 1 allotype and comprises M428L and N434S or M428L and N434A mutations or any other mutation(s) that enhance binding to a human FcRn, as described further herein.

In certain embodiments, a variant of an Fc polypeptide comprises only the specified or recited amino acid mutations (e.g. substitutions), and does not comprise any further amino acid substitutions or mutations; e.g., relative to the reference polypeptide (e.g., a wild-type Fc polypeptide or fragment thereof). For example, in some embodiments, a variant Fc polypeptide comprising the amino acid substitutions G236A_Y300L does not comprise any other amino acid substitutions; i.e., comprises an amino acid sequence that is wild-type except for G236A and Y300L.

In some embodiments, a variant of an Fc polypeptide may comprise one or more additional amino acid mutations (e.g. substitutions), which can be specified (e.g., M428L_N434S; M428L_N434A). In some embodiments, a further amino acid mutation or mutations is physically remote to the recited amino acid positions in tertiary structure, and/or is of such nature (e.g. is a conservative substitution), so that one or more function of the recited Fc variant or fragment thereof is not reduced or is reduced by no more than 50%, no more than 40%, no more than 30%, no more than 25%, no more than 20%, no more than 15%, no more than 10% or no more than 5%, or by no more than 10-fold, no more than 9-fold, no more than 8-fold, no more than 7-fold, no more than 6-fold, no more than 5-fold, no more than 4-fold, no more than 3-fold, no more than 2-fold, or no more than 1.5-fold. In some embodiments, variant of an Fc polypeptide comprises the mutations M428L and N434S or the mutations M428L and N434A, or any other mutation(s) that enhance binding to a human FcRn, including those described herein.

In some embodiments, an antibody or antigen-binding fragment (described further herein) is provided that comprises, in a(n e.g. human) IgG1 heavy chain, the amino acid mutation(s) set forth in any one of (i)-(xviii): (i) G236A, L328V, and Q295E; (ii) G236A, P230A, and Q295E; (iii) G236A, R292P, and 1377N; (iv) G236A, K334A, and Q295E; (v) G236S, R292P, and Y300L; (vi) G236A and Y300L; (vii) G236A, R292P, and Y300L; (viii) G236S, G420V, G446E, and L309T; (ix) G236A and R292P; (x) R292P and Y300L; (xi) G236A and R292P; (xii) Y300L; (xiii) E345K, G236S, L235Y, and S267E; (xiv) E272R, L309T, S219Y, and S267E; (xv) G236Y; (xvi) G236W; (xvii) F243L, G446E, P396L, and S267E; (xviii) G236A, S239D, and H268E, wherein the numbering of amino acid residues is according to the EU index as set forth in Kabat. In certain embodiments, the antibody or antigen-binding fragment is afucosylated. In some embodiments, the antibody or antigen-binding fragment further comprises one or more mutation that enhances binding to a human FcRn, such as M428L and N434S mutations or M428L and N434A mutations (EU numbering) or any other mutation(s) that enhance binding to a human FcRn, such as those described herein. In certain embodiments, the antibody or antigen-binding fragment is afucosylated. In some embodiments, the IgG1 heavy chain comprises a CH1-CH3 or a CH2-CH3 or a hinge-CH2-CH3, wherein the CH1-CH3 or CH2-CH3 or hinge-CH2-CH3 has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity (or similarity) to a wild-type human IgG1 CH1-CH3 or CH2-CH3 or hinge-CH2-CH3, respectively. In certain embodiments, an antibody or antigen-binding fragment of the present disclosure comprises an Fc variant comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity (or similarity) to the amino acid sequence set forth in any one of SEQ ID NOs:69-95.

In some embodiments, the antibody or antigen-binding fragment comprises the amino acid sequence set forth in any one of SEQ ID NOs:69-95, or a variant thereof, e.g. that further comprises one or more mutation that enhances binding to a human FcRn, such as M428L and N434S mutations or M428L and N434A mutations (EU numbering) or any other mutation(s) that enhance binding to a human FcRn, including those described herein. In some embodiments, the antibody or antigen-binding fragment comprises an amino acid sequence that differs from the amino acid sequence set forth in any one of SEQ ID NOs:69-95 only by one or more IgG1 allotype-specific mutations and/or by the presence of M428L and N434S mutations or M428L and N434A mutations or other mutation(s) that enhance binding to a human FcRn.

An antibody or antigen-binding fragment of the present disclosure can be fucosylated (e.g., comprising one or more fucosyl moiety, and typically comprising a native (wild-type) fucosylation pattern or a fucosylation pattern that includes one or more additional, or fewer, fucosyl moieties as compared to native), or can be afucosylated. In particular, native IgG1 antibodies carry a glycan site at N297, and this is typically the only site where a core fucose moiety may be found in the antibody, though some glycan sites may arise through mutation (e.g. in the variable domains) during antibody development. Fucosylation of an antibody or antigen-binding fragment, can be affected by introducing amino acid mutations to introduce or disrupt a fucosylation site (e.g. a mutation at N297, such as N297Q or N297A, to disrupt formation of a glycan that can include a core fucose moiety), though typically it is preferred to maintain N297 and the glycan thereof, such as by expressing the antibody or antigen-binding fragment in a host cell which has been genetically engineered to lack the ability (or have an inhibited or compromised ability) to fucosylate the antibody or antigen-binding fragment; by expressing the antibody or antigen-binding fragment under conditions in which a host cell is impaired in its ability to fucosylate the polypeptide (e.g., in the presence of 2-fluoro-L-fucose (2FF)), or the like. An afucosylated antibody or antigen-binding fragment can comprise no fucose moieties, or substantially no fucose moieties, and/or can be expressed by a host cell that is genetically engineered to lack the ability (or have an inhibited or compromised ability) to fucosylate the antibody or antigen-binding fragment and/or can be expressed under conditions in which a host cell is impaired in its ability to fucosylate the antibody or antigen-binding fragment (e.g., in the presence of 2-fluoro-L-fucose (2FF)). In some embodiments, an antibody or antigen-binding fragment does not comprise a core fucose moiety at Asn297. In some embodiments, afucosylated antibodies or antigen-binding fragments have increased binding to FcγRIIIA. In some contexts, addition of 2FF to a culture media comprising host cells expressing an antibody results in about 85% or more of the antibodies or antigen-binding fragments not carrying a fucose moiety. Accordingly, a plurality of antibodies or antigen-binding fragments may be described as “afucosylated” when the plurality was produced in the presence of 2FF or like reagent. In some contexts, a plurality of antibodies or antigen-binding fragments may be described as, for example, afucosylated, meaning that about 85% or more of the single antibody or antigen-binding fragment molecules of the plurality do not comprise a fucose moiety. In certain preferred embodiments, an afucosylated antibody or antigen-binding fragment or a population or a plurality thereof comprises an asparagine (N) at EU position 297. Fucosylation or lack thereof can be assessed using, for example, mass spectrometry (e.g. Electrospray mass spectrometry (ESI-MS)). In some embodiments, compositions are provided that comprise a plurality of any one or more of the presently disclosed antibodies or antigen-binding fragments, wherein the composition comprises afucosylated antibodies or antigen-binding fragments.

In certain embodiments, presently disclosed variants of IgG Fc polypeptides or fragments thereof possess one or more function that is distinct from (e.g. improved as compared to) the corresponding function of a reference Fc polypeptide that comprises the following mutation or mutations: G236A; G236S; G236A_A330L_I332E; G236A_A330L_I332E_M428L_N434S; A330L_I332E; or G236A_S239D_A330L_I332E. For example, in certain embodiments, a presently disclosed variant of an IgG Fc polypeptide or fragment thereof possesses one or more of the following properties, as compared to a reference Fc polypeptide that comprises the following mutation or mutations: G236A; G236S; G236A_A330L_I332E; G236A_A330L I332E_M428L_N434S; A330L_I332E; or G236A_S239D_A330L_I332E: increased binding (e.g. affinity) to and/or signaling via a human FcγRIIa H131; increased binding (e.g. affinity) to and/or signaling via a human FcγRIIa R131; decreased binding to (e.g. affinity) and/or signaling via human FcγRIIb; an increased ratio of binding to (e.g. affinity) and/or signaling via a human FcγRIIa (H131, R131, or both) versus the ratio of binding to or signaling via (respectively) a human FcγRIIb; increased binding (e.g. affinity) to and/or signaling via a human FcγRIIIa (V158, F158, or both); increased binding (e.g. affinity) to a human C1q; a higher Tm; an improved production titer; an improved signaling in a host cell via a FcγRIIa (H131, R131, or both); increased facilitation of ADCP and/or ADCC by human NK cells and/or human PBMCs when in the presence of antigen-presenting cells; and an improved ability to stimulate moDCs when in an immune complex with antigen.

In the present disclosure, binding of a variant Fc polypeptide or fragment may be described as increased (or “greater than”, or the like) or decreased (or “reduced” or “less than”, or the like) as compared to the binding of a comparator (e.g., to a reference wild-type IgG1 Fe, or to a reference IgG1 Fc that is wild-type except for M428L and N434S mutations or except for M428L and N434A mutations or to a variant IgG1 Fc comprising G236A_A330L_I332E mutations) to a same binding partner. Binding interactions between a variant Fc polypeptide or fragment (or an antibody or polypeptide comprising the same) and a binding partner (e.g. a human FcγR, FcRn, or C1q) can preferably be determined using an electrochemiluminescence assay, more preferably using the Meso Scale Discovery (“MSD”; mesoscale.com) platform. MSD binding assay is similar to ELISA though MSD uses electrochemiluminescence, as opposed to colorimetry, as a detection technique. Other techniques for measuring binding interactions are known and include, for example, ELISA, surface plasmon resonance (SPR), biolayer interferometry (BLI), and the like.

In some embodiments, binding includes affinity, avidity, or both. Affinity refers to the strength of a bond between a binding molecule and its binding partner. In some contexts, binding can include affinity and/or avidity. Unless otherwise indicated, avidity refers to the total binding strength of a molecule to a binding partner, and reflects binding affinity, valency of binding sites (e.g., whether an Fc polypeptide comprises one, two, or more binding sites), and, for example, whether another agent is present that can affect the binding (e.g., a non-competitive inhibitor of the Fc polypeptide).

A binding interaction between a variant molecule of the present disclosure and a binding partner can be expressed in terms of fold-change relative to the binding interaction between a reference molecule and the binding partner. For example, binding of a presently disclosed antibody comprising a variant Fc to a human FcγRIIa may be stronger than the binding of the antibody comprising a wild-type Fc to the human FcγRIIa, and the relative increased strength of the variant can be expressed in terms of fold-change (e.g., linear scale of area-under-the-curve) relative to the reference molecule binding using the same assay. For example, a variant Fc polypeptide or fragment may bind to a FcγRIIa with a 2-fold, 3-fold, 4-fold, or 5-fold greater binding strength than a reference Fc polypeptide or fragment binds to the FcγRIIa. As another example, a variant Fc polypeptide or fragment thereof may bind less strongly to a FcγRIIb as compared to a reference Fc or fragment thereof; e.g., may have a 0.9-fold binding, 0.8-fold binding, 0.7-fold binding, 0.6-fold binding, or the like, as compared to the reference Fc polypeptide or fragment thereof. It will be understood that, for example, the expression “2-fold greater binding as compared to the binding of a reference” means a 2-fold increase in binding as compared to the reference.

Moreover, binding of a variant Fc molecule of the present disclosure to two different partner molecules can be described in terms of a ratio, and this ratio can be compared to a like ratio obtained using a reference molecule with the same assay. For example, a variant Fc polypeptide may bind to a human FcγRIIa H131 five times more strongly than it binds to a human FcγRIIb, while a reference wild-type Fc polypeptide binds to FcγRIIa H131 as strongly as it binds to a human FcγRIIb. In this example, the variant Fc polypeptide can be said to have a 5:1 (binding FcγRIIIa H131:binding FcγRIIb) binding ratio, which can be compared to the 1:1 (binding FcγRIIIa H131:binding FcγRIIb) binding ratio of the reference wild-type Fc polypeptide.

Variant Fc molecules of the present disclosure may also be described in terms of ability to induce signaling in a host cell, wherein the host cell expresses or over-expresses one or more FcγR (e.g., FcγRIIa H131, FcγRIIa R131, FcγRIIb, FcγRIIIa F158, or FcγRIIIa V158) and the signaling is induced by binding of the variant molecule to the FcγR. Reporter cells useful for determining signaling include, for example, cells in which NFAT drives expression of a luciferase reporter (e.g., available from Promega®).

Unless stated otherwise, FcγRs, FcRn, and C1q as described herein are human.

In some embodiments, an antibody or antigen-binding fragment comprising a variant Fc polypeptide or fragment is preferably capable of inducing one or more of: antibody-dependent cell cytotoxicity (ADCC), antibody-dependent cellular phagocytosis (ADCP); and complement-dependent cytotoxicity. Assays for measuring these functions are known.

In some embodiments, a variant Fc polypeptide or fragment (or antibody or antigen-binding fragment comprising the same) preferably has comparable binding to a human FcRn (e.g., at pH 6.0) and/or a comparable in vivo half-life in a mammal as compared to a reference Fc polypeptide, fragment, antibody, or antigen-binding fragment, respectively.

In some embodiments, a variant Fc polypeptide or fragment (or antibody or antigen-binding fragment comprising the same) preferably has increased binding to a human FcRn (e.g., at pH 6.0) and/or increased in vivo half-life in a mammal as compared to a reference Fc polypeptide, fragment, antibody, or antigen-binding fragment, respectively.

In some embodiments, a variant Fc polypeptide or fragment (or antibody or antigen-binding fragment comprising the same) preferably has a melting temperature (Tm) that is less than 12° C., less than 11° C., less than 10° C., less than 9° C., less than 8° C., less than 7° C., less than 6° C., less than 5° C., less than 4° C., less than 3° C., less than 2° C., or less than 1° C. below the Tm of a reference Fc polypeptide (or antibody or antigen-binding fragment comprising the same), or has a Tm that is higher than the Tm of the reference Fc polypeptide or fragment (or polypeptide or antibody comprising the same). In some embodiments, the reference polypeptide or fragment is or comprises a wild-type human Fc polypeptide (or antibody comprising the same).

In some embodiments, a variant Fc polypeptide or fragment (or antibody or antigen-binding fragment comprising the same) has a melting temperature that is higher than the melting temperature of a reference Fc polypeptide or fragment (or antibody or antigen-binding fragment comprising the same) that comprises the mutations G236A, A330L, I332E, and, optionally, M428L and N434S.

In some embodiments, a variant Fc polypeptide or fragment (or antibody or antigen-binding fragment comprising the same) is preferably capable of being produced in a host cell line (e.g., a CHO cell line) at least about as efficiently (e.g., produces at least about the same titer and/or within less than 0.1-fold, less than 0.09-fold, less than 0.08-fold, less than 0.07-fold, less than 0.06-fold, less than 0.05-fold, less than 0.04-fold, less than 0.03-fold, less than 0.02-fold, or less than 0.02-fold less) as compared to a reference Fc polypeptide or fragment (or antibody or antigen-binding fragment comprising the same).

In certain embodiments, an anti-NA antibody or antigen-binding fragment of the present disclosure comprises a variant of: (i) an IgG CH2 polypeptide or (ii) an IgG Fc polypeptide or a fragment thereof, wherein the variant comprises an alanine (A) at EU position 236, a valine (V) at EU position 328, and a glutamic acid (E) at EU position 295. In some embodiments, the IgG Fc polypeptide or fragment thereof comprises an (e.g., otherwise wild-type) IgG1 Fc polypeptide or fragment thereof (“GALVQE”). In some embodiments, the antibody or antigen-binding fragment further comprises the mutations M428L and N434S, or the mutations M428L and N434A, or any other mutation(s) that enhance binding to a human FcRn, such as those described herein. In certain embodiments, the antibody or antigen-binding fragment is afucosylated.

In certain other embodiments, an anti-NA antibody or antigen-binding fragment of the present disclosure comprises a variant of (i) an IgG hinge-CH2 polypeptide; or (ii) an IgG hinge-Fc polypeptide or a fragment thereof, wherein the variant comprises an alanine (A) at EU position 236, an alanine (A) at EU position 230, and a glutamic acid (E) at EU position 295. In some embodiments, the IgG Fc polypeptide or fragment thereof comprises an (e.g., otherwise wild-type) IgG1 Fc polypeptide or fragment thereof (“GAPAQE”). In some embodiments, the antibody or antigen-binding fragment further comprises the mutations M428L and N434S, or the mutations M428L and N434A, or any other mutation(s) that enhance binding to a human FcRn, such as those described herein. In certain embodiments, the antibody or antigen-binding fragment is afucosylated.

In certain other embodiments, an anti-NA antibody or antigen-binding fragment of the present disclosure comprises a variant of: an IgG Fc polypeptide or a fragment thereof, wherein the variant comprises an alanine (A) at EU position 236, a proline (P) at EU position 292, and an asparagine (N) at EU position 377. In some embodiments, the IgG Fc polypeptide or fragment thereof comprises an (e.g., otherwise wild-type) IgG1 Fc polypeptide or fragment thereof (“GARPIN”). In some embodiments, the antibody or antigen-binding fragment further comprises the mutations M428L and N434S, or the mutations M428L and N434A, or any other mutation(s) that enhance binding to a human FcRn, such as those described herein. In certain embodiments, the antibody or antigen-binding fragment is afucosylated.

In certain other embodiments, an anti-NA antibody or antigen-binding fragment comprises a variant of: (i) an IgG CH2 polypeptide or (ii) an IgG Fc polypeptide or a fragment thereof, wherein the variant comprises an alanine (A) at EU position 236, an alanine (A) at EU position 334, and a glutamic acid (E) at EU position 295. In some embodiments, the IgG Fc polypeptide or fragment thereof comprises an (e.g., otherwise wild-type) IgG1 Fc polypeptide or fragment thereof (“GAKAQE”). In some embodiments, the antibody or antigen-binding fragment further comprises the mutations M428L and N434S, or the mutations M428L and N434A, or any other mutation(s) that enhance binding to a human FcRn, such as those described herein. In certain embodiments, the antibody or antigen-binding fragment is afucosylated.

In certain other embodiments, an anti-NA antibody or antigen-binding fragment of the present disclosure comprises a variant of: (i) an IgG CH2 polypeptide or (ii) an IgG Fc polypeptide or a fragment thereof, wherein the variant comprises a serine (S) at EU position 236, a proline (P) at EU position 292, and a leucine (L) at EU position 300. In some embodiments, the IgG Fc polypeptide or fragment thereof comprises an (e.g., otherwise wild-type) IgG1 Fc polypeptide or fragment thereof (“GSRPYL”). In some embodiments, the antibody or antigen-binding fragment further comprises the mutations M428L and N434S, or the mutations M428L and N434A, or any other mutation(s) that enhance binding to a human FcRn, such as those described herein. In certain embodiments, the antibody or antigen-binding fragment is afucosylated.

In certain other embodiments, an anti-NA antibody or antigen-binding fragment of the present disclosure comprises a variant of: (i) an IgG CH2 polypeptide or (ii) an IgG Fc polypeptide or a fragment thereof, wherein the variant comprises an alanine (A) at EU position 236, a proline (P) at EU position 292, and a leucine (L) at EU position 300. In some embodiments, the IgG Fc polypeptide or fragment thereof comprises an (e.g., otherwise wild-type) IgG1 Fc polypeptide or fragment thereof (“GARPYL”). In some embodiments, the antibody or antigen-binding fragment further comprises the mutations M428L and N434S, or the mutations M428L and N434A, or any other mutation(s) that enhance binding to a human FcRn, such as those described herein. In certain embodiments, the antibody or antigen-binding fragment is afucosylated.

In certain other embodiments, an anti-NA antibody or antigen-binding fragment of the present disclosure comprises a variant of: (i) an IgG CH2 polypeptide or (ii) an IgG Fc polypeptide or a fragment thereof, wherein the variant comprises an alanine (A) at EU position 236 and a leucine (L) at EU position 300. In some embodiments, the IgG Fc polypeptide or fragment thereof comprises an (e.g., otherwise wild-type) IgG1 Fc polypeptide or fragment thereof (“GAYL”). In some embodiments, the antibody or antigen-binding fragment further comprises the mutations M428L and N434S, or the mutations M428L and N434A, or any other mutation(s) that enhance binding to a human FcRn, such as those described herein. In certain embodiments, the antibody or antigen-binding fragment is afucosylated.

In certain other embodiments, an anti-NA antibody or antigen-binding fragment of the present disclosure comprises a variant of (i) an IgG CH2 polypeptide; or (ii) an IgG Fc polypeptide or a fragment thereof, wherein the variant comprises an alanine (A) at EU position 236, an aspartic acid (D) at EU position 239, and a glutamic acid (E) at EU position 268. In some embodiments, the IgG Fc polypeptide or fragment thereof comprises an (e.g., otherwise wild-type) IgG1 Fc polypeptide or fragment thereof (“GASDHE”). In some embodiments, the antibody or antigen-binding fragment further comprises the mutations M428L and N434S, or the mutations M428L and N434A, or any other mutation(s) that enhance binding to a human FcRn, such as those described herein. In certain embodiments, the antibody or antigen-binding fragment is afucosylated.

In some embodiments, the antibody or antigen-binding fragment has increased binding to a human FcγRIIa and/or has decreased binding to a human FcγRIIb, as compared to the binding of a reference antibody or antigen-binding fragment to the human FcγRIIa or the human FcγRIIb, respectively, wherein, optionally, binding is as determined using an electrochemiluminescence assay, further optionally Meso Scale Discovery.

In certain embodiments, the increased binding to a human FcγRIIa comprises more than 1-fold, at least 2-fold, at least 3-fold, at least 4-fold, at least 5-fold, at least 6-fold, at least 7-fold, at least 8-fold, at least 9-fold, or at least 10-fold greater binding to the human FcγRIIa as compared to the binding of a reference antibody or antigen-binding fragment (optionally comprising a wild-type human IgG (e.g. IgG1) Fc polypeptide or a fragment thereof) to the human FcγRIIa.

In some embodiments, the human FcγRIIa comprises H131 and, optionally, the increased binding to the human FcγRIIa H131 comprises at least 4-fold, at least 5-fold, at least 6-fold, at least 7-fold, at least 8-fold, at least 9-fold, or at least 10-fold greater binding to the human FcγRIIa H131 as compared to the binding of a reference antibody or antigen-binding fragment (optionally comprising a wild-type human IgG (e.g. IgG1) Fc polypeptide or a fragment thereof) to the human FcγRIIa H131.

In some embodiments, the human FcγRIIa comprises R131 and, optionally, the increased binding to the human FcγRIIa R131 comprises more than 1-fold, at least 2-fold, at least 3-fold, at least 4-fold, at least 5-fold, at least 6-fold, at least 7-fold, at least 8-fold, at least 9-fold, or at least 10-fold greater binding to the human FcγRIIa R131 as compared to the binding of a reference antibody or antigen-binding fragment (optionally comprising a wild-type human IgG (e.g. IgG1) Fc polypeptide or a fragment thereof) to the human FcγRIIa R131.

In some embodiments, the decreased binding to a human FcγRIIb comprises less than 0.9-fold, less than 0.8-fold, less than 0.7-fold, less than 0.6-fold, or between 0.5-fold and 0.9-fold, of the binding of a reference antibody or antigen-binding fragment (optionally comprising a wild-type human IgG (e.g. IgG1) Fc polypeptide or a fragment thereof) to the human FcγRIIb.

In any of the presently disclosed embodiments, (1) a ratio of (i) the binding of the antibody or antigen-binding fragment to a human FcγRIIa to (ii) the binding of the antibody or antigen-binding fragment, respectively, to a human FcγRIIb is greater than (2) a ratio of (iii) the binding of a reference polypeptide to the human FcγRIIa to (iv) the binding of the reference antibody or antigen-binding fragment to the human FcγRIIb, wherein the reference antibody or antigen-binding fragment optionally comprises a wild-type human IgG (e.g. IgG1) Fc polypeptide or a fragment thereof, wherein, optionally, binding is as determined using an electrochemiluminescence assay, further optionally Meso Scale Discovery. In some embodiments, the human FcγRIIa comprises H131, R131, or both. In some embodiments, the ratio in (1) is more than 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, or at least 14-fold greater than the ratio in (2). Also provided is an anti-NA antibody or antigen-binding fragment of the present disclosure comprising a variant of: (i) an IgG CH2 polypeptide or (ii) an IgG Fc polypeptide or a fragment thereof, wherein the variant comprises an alanine (A) at EU position 236 and a leucine (L) at EU position 300. In some embodiments, the IgG Fc polypeptide or fragment thereof comprises an (e.g., otherwise wild-type) IgG1 Fc polypeptide or fragment thereof (“GAYL”). In certain further embodiments, the mutations M428L and N434S, or the mutations M428L and N434A, or any other mutation(s) that enhance binding to a human FcRn, such as those described herein, are present. In certain embodiments, the antibody or antigen-binding fragment is afucoyslated.

In some embodiments, the antibody or antigen-binding fragment has increased binding to a human FcγRIIa as compared to the binding of a reference antibody or antigen-binding fragment to the human FcγRIIa, wherein, optionally, binding is as determined using an electrochemiluminescence assay, further optionally Meso Scale Discovery.

In some embodiments, the increased binding to a human FcγRIIa comprises at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, or at least 18-fold greater binding to the human FcγRIIa as compared to the binding of a reference antibody or antigen-binding fragment (optionally comprising a wild-type human IgG (e.g. IgG1) Fc polypeptide or a fragment thereof) to the human FcγRIIa.

In some embodiments, the human FcγRIIa comprises H131 and, optionally, the increased binding to the human FcγRIIa H131 comprises at least 4-fold, at least 5-fold, at least 6-fold, at least 7-fold, at least 8-fold, at least 9-fold, or at least 10-fold, at least 11-fold, at least 12-fold, at least 13-fold, at least 14-fold, at least 15-fold, at least 16-fold, at least 17-fold, or at least 18-fold greater binding to the human FcγRIIa H131 as compared to the binding of a reference antibody or antigen-binding fragment (optionally comprising a wild-type human IgG (e.g. IgG1) Fc polypeptide or a fragment thereof) to the human FcγRIIa H131.

In some embodiments, the human FcγRIIa comprises R131 and, optionally, the increased binding to the human FcγRIIa R131 comprises at least 4-fold greater binding to the human FcγRIIa R131 as compared to the binding of a reference antibody or antigen-binding fragment (optionally comprising a wild-type human IgG (e.g. IgG1) Fc polypeptide or a fragment thereof) to the human FcγRIIa R131.

In certain embodiments, (1) a ratio of (i) the binding of the antibody or antigen-binding fragment to a human FcγRIIa to (ii) the binding of the antibody or antigen-binding fragment, respectively, to a human FcγRIIb is greater than (2) a ratio of (iii) the binding of a reference antibody or antigen-binding fragment to the human FcγRIIa to (iv) the binding of the reference polypeptide to the human FcγRIIb, wherein the reference antibody or antigen-binding fragment comprises a wild-type human IgG Fc polypeptide or a fragment thereof. In certain embodiments, the human FcγRIIa comprises H131, R131, or both. In further embodiments, the ratio in (1) is at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, or at least 17-fold greater than the ratio in (2).

Also provided is an anti-NA antibody or antigen-binding fragment of the present disclosure that comprises a variant of: (i) an IgG CH2 polypeptide or (ii) an IgG Fc polypeptide or a fragment thereof, wherein the variant comprises an alanine (A) at EU position 236, a proline (P) at EU position 292, and a leucine (L) at EU position 300. In some embodiments, the IgG Fc polypeptide or fragment thereof comprises an (e.g., otherwise wild-type) IgG1 Fc polypeptide or fragment thereof (“GARPYL”). In certain further embodiments, the mutations M428L and N434S, or the mutations M428L and N434A, or any other mutation(s) that enhance binding to a human FcRn, such as those described herein, are present. In certain embodiments, the antibody or antigen-binding fragment is afucoyslated.

In certain embodiments, the antibody or antigen-binding fragment has increased binding to a human FcγRIIIa, as compared to the binding of a reference antibody or antigen-binding fragment to the human FcγRIIIa, wherein, optionally, binding is as determined using an electrochemiluminescence assay, further optionally Meso Scale Discovery.

In some embodiments, the increased binding to a human FcγRIIa comprises at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, or at least 14-fold greater binding to the human FcγRIIa as compared to the binding of a reference antibody or antigen-binding fragment optionally comprising a wild-type human IgG Fc polypeptide or a fragment thereof to the human FcγRIIa.

In some embodiments, the human FcγRIIa comprises H131 and, optionally, the increased binding to the human FcγRIIa H131 comprises at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, or at least 14-fold greater binding to the human FcγRIIa H131 as compared to the binding of a reference antibody or antigen-binding fragment optionally comprising a wild-type human IgG Fc polypeptide or a fragment thereof to the human FcγRIIa H131.

In some embodiments, the human FcγRIIa comprises R131 and, optionally, the increased binding to the human FcγRIIa H131 comprises at least 2-fold greater binding to the human FcγRIIa R131 as compared to the binding of a reference antibody or antigen-binding fragment optionally comprising a wild-type human IgG Fc polypeptide or a fragment thereof to the human FcγRIIa R131.

In certain embodiments, (1) a ratio of (i) the binding of the antibody or antigen-binding fragment to a human FcγRIIa to (ii) the binding of the antibody or antigen-binding fragment, respectively, to a human FcγRIIb is greater than (2) a ratio of (iii) the binding of a reference antibody or antigen-binding fragment to the human FcγRIIa to (iv) the binding of the reference antibody or antigen-binding fragment to the human FcγRIIb, wherein the reference antibody or antigen-binding fragment optionally comprises a wild-type human IgG Fc polypeptide or a fragment thereof, wherein, optionally, binding is as determined using an electrochemiluminescence assay, further optionally Meso Scale Discovery. In some embodiments, the human FcγRIIa comprises H131, R131, or both. In some embodiments, the ratio in (1) is at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, or at least 15-fold greater than the ratio in (2).

In certain embodiments, the antibody or antigen-binding fragment has increased binding to a human FcγRIIIa, as compared to the binding of a reference antibody or antigen-binding fragment to the human FcγRIIIa, wherein, optionally, binding is as determined using an electrochemiluminescence assay, further optionally Meso Scale Discovery. In some embodiments, the human FcγRIII comprises V158, F158, or both. In certain further embodiments, the increased binding to a human FcγRIIIa comprises greater than 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3.0 fold, at least 3.1-fold, at least 3.2-fold, at least 3.3-fold, at least 3.4-fold, at least 3.5-fold, at least 3.6-fold, or at least 3.7-fold greater binding to the human FcγRIIIa as compared to the binding of a reference antibody or antigen-binding fragment optionally comprising a wild-type human IgG Fc polypeptide or a fragment thereof to the human FcγRIIIa.

In certain embodiments, the antibody or antigen-binding fragment is capable of binding to a human complement component 1q (C1q), wherein, optionally, binding is as determined using an electrochemiluminescence assay, further optionally Meso Scale Discovery.

Also provided is an anti-NA antibody or antigen-binding fragment of the present disclosure that comprises a variant of an IgG Fc polypeptide, wherein the variant comprises a seine (S) at EU position 236, a valine (V) at EU position 420, a glutamic acid (E) at EU position 446, and a threonine (T) at EU position 309. In some embodiments, the IgG Fc polypeptide or fragment thereof comprises an (e.g., otherwise wild-type) IgG1 Fc polypeptide or fragment thereof (“GSGVGELT”). In certain further embodiments, the mutations M428L and N434S, or the mutations M428L and N434A, or any other mutation(s) that enhance binding to a human FcRn, such as those described herein, are present. In certain embodiments, the antibody or antigen-binding fragment is afucoyslated.

Also provided is an anti-NA antibody or antigen-binding fragment of the present disclosure that comprises a variant of: (i) an IgG CH2 polypeptide or (ii) an IgG Fc polypeptide, wherein the variant comprises an alanine (A) at EU position 236 and a proline (P) at EU position 292. In some embodiments, the antibody or antigen-binding fragment comprises an (e.g., otherwise wild-type) IgG1 Fc polypeptide or fragment thereof (“GARP”). In certain further embodiments, the mutations M428L and N434S, or the mutations M428L and N434A, or any other mutation(s) that enhance binding to a human FcRn, such as those described herein, are present. In certain embodiments, the antibody or antigen-binding fragment is afucoyslated.

In certain embodiments, the antibody or antigen-binding fragment has decreased binding to a human FcγRIIb as compared to the binding of a reference antibody or antigen-binding fragment to the human FcγRIIb, wherein, optionally, binding is as determined using an electrochemiluminescence assay, further optionally Meso Scale Discovery. In some embodiments, the decreased binding to a human FcγRIIb comprises less than 0.9-fold, less than 0.8-fold, less than 0.7-fold, less than 0.6-fold, less than 0.5-fold, or less than 0.4-fold as compared to the binding of a reference antibody or antigen-binding fragment optionally comprising a wild-type human IgG Fc polypeptide or a fragment thereof to the human FcγRIIb.

In further embodiments, the antibody or antigen-binding fragment has increased binding to a human FcγRIIa as compared to the binding of a reference antibody or antigen-binding fragment to the human FcγRIIa, wherein, optionally, binding is as determined using an electrochemiluminescence assay, further optionally Meso Scale Discovery.

In some embodiments, the increased binding to the human FcγRIIa comprises greater than 1-fold, at least 2-fold, at least 3-fold, at least 4-fold, or at least 5-fold greater binding to the human FcγRIIa as compared to the binding of a reference antibody or antigen-binding fragment comprising a wild-type human IgG Fc polypeptide or a fragment thereof to the human FcγRIIa.

In certain embodiments, the human FcγRIIa comprises H131, R131, or both.

In some embodiments, (1) a ratio of (i) the binding of the antibody or antigen-binding fragment to a human FcγRIIa to (ii) the binding of the antibody or antigen-binding fragment, respectively, to a human FcγRIIb is greater than (2) a ratio of (iii) the binding of a antibody or antigen-binding fragment to the human FcγRIIa to (iv) the binding of the reference antibody or antigen-binding fragment to the human FcγRIIb, wherein the reference antibody or antigen-binding fragment optionally comprises a wild-type human IgG Fc polypeptide or a fragment thereof, wherein, optionally, binding is as determined using an electrochemiluminescence assay, further optionally Meso Scale Discovery. In some embodiments, the human FcγRIIa comprises H131, R131, or both. In certain embodiments, the ratio in (1) is at least 2-fold, at least 3-fold, at least 4-fold, at least 5-fold, at least 6-fold, at least 7-fold, at least 8-fold, at least 10-fold, at least 11-fold, or at least 12-fold greater than the ratio in (2).

Also provided is an anti-NA antibody or antigen-binding fragment of the present disclosure that comprises a variant of: (i) an IgG CH2 polypeptide or (ii) an IgG Fc polypeptide, wherein the variant comprises a proline (P) at EU position 292 and a leucine (L) at EU position 300, and wherein, optionally, variant and, further optionally, the antibody or antigen-binding fragment has increased binding to a human FcγRIIIa with as compared to the binding of a reference antibody or antigen-binding fragment to the human FcγRIIIa, wherein, optionally, the binding is as determined using an electrochemiluminescence assay, further optionally Meso Scale Discovery. In some embodiments, the antibody or antigen-binding fragment comprises an (e.g., otherwise wild-type) IgG1 CH2 polypeptide or IgG Fc polypeptide (“RPYL”). In certain further embodiments, the mutations M428L and N434S, or the mutations M428L and N434A, or any other mutation(s) that enhance binding to a human FcRn, such as those described herein, are present. In certain embodiments, the antibody or antigen-binding fragment is afucoyslated.

In certain embodiments, the human FcγRIIIa comprises V158, F158, or both, and wherein the increased binding to the human FcγRIIIa comprises at least 4-fold, at least 4.5-fold, at least 5-fold, at least 5.1-fold, or at least 5.2-fold greater binding as compared to the binding of a reference antibody or antigen-binding fragment optionally comprising a wild-type human IgG Fc polypeptide or a fragment thereof to the human FcγRIIa.

Also provided is an anti-NA antibody or antigen-binding fragment of the present disclosure that comprises a variant of: (i) an IgG CH2 polypeptide or (ii) an IgG Fc polypeptide or a fragment thereof, wherein the variant comprises a leucine (L) at EU position 300. In some embodiments, the IgG CH2 polypeptide or IgG Fc polypeptide or fragment thereof comprises an (e.g., otherwise wild-type) IgG1 Fc polypeptide or fragment thereof (“YL”). In certain further embodiments, the mutations M428L and N434S, or the mutations M428L and N434A, or any other mutation(s) that enhance binding to a human FcRn, such as those described herein, are present. In certain embodiments, the antibody or antigen-binding fragment is afucoyslated.

Also provided is an anti-NA antibody or antigen-binding fragment of the present disclosure that comprises a variant of: an IgG Fc polypeptide or a fragment thereof, wherein the variant comprises a lysine (K) at EU position 345, a serine (S) at EU position 236, tyrosine (Y) at EU position 235, and a glutamic acid (E) at EU position 267. In some embodiments, the IgG Fc polypeptide or fragment thereof comprises an (e.g., otherwise wild-type) IgG1 Fc polypeptide or fragment thereof (“GSEKLYSE”). In certain further embodiments, the mutations M428L and N434S, or the mutations M428L and N434A, or any other mutation(s) that enhance binding to a human FcRn, such as those described herein, are present. In certain embodiments, the antibody or antigen-binding fragment is afucoyslated.

Also provided is an anti-NA antibody or antigen-binding fragment of the present disclosure that comprises a variant of: (i) an IgG hinge-CH2 polypeptide or (ii) an IgG hinge-Fc polypeptide or a fragment thereof, wherein the variant comprises an arginine (R) at EU position 272, a threonine (T) at EU position 309, a tyrosine (Y) at EU position 219, and a glutamic acid (E) at EU position 267. In some embodiments, the IgG hinge-CH2 polypeptide or an IgG hinge-Fc polypeptide or a fragment thereof comprises an (e.g. otherwise wild-type) IgG1 hinge-CH2 polypeptide or IgG hinge-Fc polypeptide or a fragment thereof (“SYSEERLT”). In certain further embodiments, the mutations M428L and N434S, or the mutations M428L and N434A, or any other mutation(s) that enhance binding to a human FcRn, such as those described herein, are present. In certain embodiments, the antibody or antigen-binding fragment is afucoyslated.

Also provided is an anti-NA antibody or antigen-binding fragment of the present disclosure that comprises a variant of: (i) an IgG CH2 polypeptide or (ii) an IgG Fc polypeptide or a fragment thereof, wherein the variant comprises a tyrosine (Y) at EU position 236. In some embodiments, the IgG Fc polypeptide or fragment thereof comprises an (e.g., otherwise wild-type) IgG1 Fc polypeptide or fragment thereof (“GY”). In certain further embodiments, the mutations M428L and N434S, or the mutations M428L and N434A, or any other mutation(s) that enhance binding to a human FcRn, such as those described herein, are present. In certain embodiments, the antibody or antigen-binding fragment is afucoyslated.

Also provided is an anti-NA antibody or antigen-binding fragment of the present disclosure that comprises a variant of: (i) an IgG CH2 polypeptide or (ii) an IgG Fc polypeptide or a fragment thereof, wherein the variant comprises a tryptophan (W) at EU position 236. In some embodiments, the IgG CH2 polypeptide or (ii) an IgG Fc polypeptide or a fragment thereof comprises an (e.g., otherwise wild-type) IgG1 CH2 polypeptide or Fc polypeptide or fragment thereof (“GW”). In certain further embodiments, the mutations M428L and N434S, or the mutations M428L and N434A, or any other mutation(s) that enhance binding to a human FcRn, such as those described herein, are present. In certain embodiments, the antibody or antigen-binding fragment is afucoyslated.

Also provided is an anti-NA antibody or antigen-binding fragment of the present disclosure that comprises a variant of: (i) an IgG CH2 polypeptide or (ii) an IgG Fc polypeptide or a fragment thereof, wherein the variant comprises an alanine (A) at EU position 236, wherein the IgG Fc polypeptide or fragment thereof, and optionally the polypeptide, is afucosylated, and wherein, further optionally, the variant comprises a leucine (L) at EU position 330 and a glutamic acid (E) at EU position 332, wherein, still further optionally, the variant does not comprise an aspartic acid (D) at EU position 239, and, even further optionally, comprises a serine (S) at EU position 239. In some embodiments, the IgG CH2 polypeptide or (ii) an IgG Fc polypeptide or a fragment thereof comprises an (e.g., otherwise wild-type) IgG1 CH2 polypeptide or Fc polypeptide or fragment thereof (“GA-afuc” or “GAALIE-afuc”, respectively). In certain further embodiments, the mutations M428L and N434S, or the mutations M428L and N434A, or any other mutation(s) that enhance binding to a human FcRn, such as those described herein, are present.

Also provided is an anti-NA antibody or antigen-binding fragment of the present disclosure that comprises a variant of: an IgG Fc polypeptide or a fragment thereof, wherein the variant comprises a leucine (L) at EU position 243, a glutamic acid (E) at EU position 446, a leucine (L) at EU position 396, and a glutamic acid (E) at EU position 267. In some embodiments, the IgG Fc polypeptide or fragment thereof comprises an (e.g., otherwise wild-type) IgG1 Fc polypeptide or fragment thereof (“FLSEPLGE”). In certain further embodiments, the mutations M428L and N434S, or the mutations M428L and N434A, or any other mutation(s) that enhance binding to a human FcRn, such as those described herein, are present. In certain embodiments, the antibody or antigen-binding fragment is afucoyslated.

Also provided is an anti-NA antibody or antigen-binding fragment that comprises a variant of: (i) an IgG CH2 polypeptide or (ii) an IgG Fc polypeptide or a fragment thereof, wherein the variant comprises an alanine (A) at EU position 236, an aspartic acid (D) at EU position 239, a glutamic acid (E) and EU position 332, a leucine (L) at EU position 428, and a seine (S) or an alanine (A) at EU position 434. In some embodiments, the IgG Fc polypeptide or fragment thereof comprises an (e.g., otherwise wild-type) IgG1 Fc polypeptide or fragment thereof (“GASDIEMLNS” or “GASDIEMLNA”).

In some embodiments, an antibody or antigen-binding fragment of the present disclosure comprises a variant of an (e.g. IgG1) IgG Fc polypeptide, wherein the variant comprises the following mutations, according to EU numbering: (i) M428L, N434S, G236A, L328V, and Q295E; (ii) M428L, N434S, G236A, R292P, and I377N; (iii) M428L, N434S, G236A, and Y300L; (iv) M428L, N434S, G236A, R292P, and Y300L; (v) M428L, N434S, G236A, L328V, and Q295E, wherein the antibody or antigen-binding fragment is afucosylated; (vi) M428L, N434S, G236A, R292P, and 1377N, wherein the antibody or antigen-binding fragment is afucosylated; (vii) M428L, N434S, G236A, and Y300L, wherein the antibody or antigen-binding fragment is afucosylated; or (viii) M428L, N434S, G236A, R292P, and Y300L, wherein the antibody or antigen-binding fragment is afucosylated. In some embodiments, the variant of an (e.g. IgG1) IgG Fc polypeptide comprises amino acid substitutions that consist essentially of the substitution mutations in (i), (ii), (iii), (iv), (v), (vi), (vii), or (viii) above. In some embodiments, the antibody or antigen-binding fragment comprises a kappa light chain.

In some embodiments, an antibody or antigen-binding fragment of the present disclosure comprises a variant of an (e.g. IgG1) IgG Fc polypeptide, wherein the variant comprises the following mutations, according to EU numbering: (i) M428L, N434A, G236A, L328V, and Q295E; (ii) M428L, N434A, G236A, R292P, and I377N; (iii) M428L, N434A, G236A, and Y300L; (iv) M428L, N434A, G236A, R292P, and Y300L; (v) M428L, N434A, G236A, L328V, and Q295E, wherein the antibody or antigen-binding fragment is afucosylated; (vi) M428L, N434A, G236A, R292P, and 1377N, wherein the antibody or antigen-binding fragment is afucosylated; (vii) M428L, N434A, G236A, and Y300L, wherein the antibody or antigen-binding fragment is afucosylated; or (viii) M428L, N434A, G236A, R292P, and Y300L, wherein the antibody or antigen-binding fragment is afucosylated. In some embodiments, the variant of an IgG Fc polypeptide comprises amino acid substitutions that consist essentially of the substitution mutations in (i), (ii), (iii), (iv), (v), (vi), (vii), or (viii) above. In some embodiments, the antibody comprises a kappa light chain. In certain embodiments, the antibody or antigen-binding fragment is capable of eliciting continued protection in vivo in a subject even once no detectable levels of the antibody or antigen-binding fragment can be found in the subject (i.e., when the antibody or antigen-binding fragment has been cleared from the subject following administration). Such protection is referred to herein as a vaccinal effect. Without wishing to be bound by theory, it is believed that dendritic cells can internalize complexes of antibody and antigen and thereafter induce or contribute to an endogenous immune response against antigen. In certain embodiments, an antibody or antigen-binding fragment comprises one or more modifications, such as, for example, mutations in the Fc comprising G236A, A330L, and I332E, that are capable of activating dendritic cells that may induce, e.g., T cell immunity to the antigen. In any of the presently disclosed embodiments, the antibody or antigen-binding fragment comprises a Fc polypeptide or a fragment thereof, including a CH2 (or a fragment thereof, a CH3 (or a fragment thereof), or a CH2 and a CH3, wherein the CH2, the CH3, or both can be of any isotype and may contain amino acid substitutions or other modifications as compared to a corresponding wild-type CH2 or CH3, respectively. In certain embodiments, a Fc of the present disclosure comprises two CH2-CH3 polypeptides that associate to form a dimer.

Non-limiting examples of heavy chain or Fc amino sequences—including certain sequences comprising amino acid substitution mutations as disclosed herein—are provided in SEQ ID NOs:69-95. In certain embodiments, an anti-NA antibody or antigen-binding fragment of the present disclosure comprises a heavy chain comprising an amino acid sequence having at least 85%, at least 86%, at least 87%, at least 88%, at least 89%, at least 90%, at least 91%, at least 93%, at least 94%, at least 95%, at least 96%, at least 97%, at least 98%, or at least 99% identity (or similarity) to, or comprising or consisting of, the amino acid sequence set forth in any one of SEQ ID NOs:69-95.

In any of the presently disclosed embodiments, the antibody or antigen-binding fragment can be monoclonal. The term “monoclonal antibody” (mAb) as used herein refers to an antibody obtained from a population of substantially homogeneous antibodies, i.e., individual antibodies comprising the population are identical except for possible naturally occurring mutations that may be present, in some cases in minor amounts. Monoclonal antibodies are highly specific, being directed against a single antigenic site. Furthermore, in contrast to polyclonal antibody preparations that include different antibodies directed against different epitopes, each monoclonal antibody is directed against a single epitope of the antigen. In addition to their specificity, the monoclonal antibodies are advantageous in that they may be synthesized uncontaminated by other antibodies. The term “monoclonal” is not to be construed as requiring production of the antibody by any particular method. For example, monoclonal antibodies useful in the present invention may be prepared by the hybridoma methodology first described by Kohler et al., Nature 256:495 (1975), or may be made using recombinant DNA methods in bacterial, eukaryotic animal, or plant cells (see, e.g., U.S. Pat. No. 4,816,567). Monoclonal antibodies may also be isolated from phage antibody libraries using the techniques described in Clackson et al., Nature, 352:624-628 (1991) and Marks et al., J Mol. Biol., 222:581-597 (1991), for example. Monoclonal antibodies may also be obtained using methods disclosed in PCT Publication No. WO 2004/076677A2.

Antibodies and antigen-binding fragments of the present disclosure include “chimeric antibodies” in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, U.S. Pat. Nos. 4,816,567; 5,530,101 and 7,498,415; and Morrison et al., Proc. Natl. Acad. Sci. USA, 81:6851-6855 (1984)). For example, chimeric antibodies may comprise human and non-human residues. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). Chimeric antibodies also include primatized and humanized antibodies.

A “humanized antibody” is generally considered to be a human antibody that has one or more amino acid residues introduced into it from a source that is non-human. These non-human amino acid residues are typically taken from a variable domain. Humanization may be performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting non-human variable sequences for the corresponding sequences of a human antibody. Accordingly, such “humanized” antibodies are chimeric antibodies (U.S. Pat. Nos. 4,816,567; 5,530,101 and 7,498,415) wherein substantially less than an intact human variable domain has been substituted by the corresponding sequence from a non-human species. In some instances, a “humanized” antibody is one which is produced by a non-human cell or animal and comprises human sequences, e.g., Hc domains.

A “human antibody” is an antibody containing only sequences that are present in an antibody that is produced by a human (i.e., sequences that are encoded by human antibody-encoding genes). However, as used herein, human antibodies may comprise residues or modifications not found in a naturally occurring human antibody (e.g., an antibody that is isolated from a human), including those modifications and variant sequences described herein. These are typically made to further refine or enhance antibody performance. In some instances, human antibodies are produced by transgenic animals. For example, see U.S. Pat. Nos. 5,770,429; 6,596,541 and 7,049,426.

In certain embodiments, an antibody or antigen-binding fragment of the present disclosure is chimeric, humanized, or human.

In some embodiments, pharmacokinetic (“PK”) parameters are used to describe or characterize an antibody or antigen-binding fragment provided herein. Details regarding collection of antibody serum concentrations for purpose of evaluating PK parameters are described in association with the Examples herein. The term “t_{1/2}” or “half-life” refers to the elimination half-life of the antibody or antigen-binding fragment included in the pharmaceutical composition administered to a subject. The term “Cₗₐₛₜ” generally refers to the last measurable plasma concentration (i.e., subsequent thereto, the substance is not present at a measurable concentration in plasma).

In some embodiments, the VH and VL of an antibody or antigen-binding fragment comprise the CDRH1, CDRH2, CDRH3, CDRL1, CDRL2, and CDRL3 amino acid sequences, as determined by the IMGT numbering system or method, of the VH and VL set forth in SEQ ID NOs. (i) 45 and 37, respectively; (ii) 46 and 37, respectively; (iii) 48 and 37, respectively; (iv) 50 and 37, respectively; (v) 52 and 37, respectively; (vi) 54 and 37, respectively; (vii) 56 and 37, respectively; (viii) 58 and 37, respectively; (ix) 60 and 37, respectively; (x) 63 and 37, respectively; (xi) 65 and 37, respectively; (xii) 45 and 66, respectively; (xiii) 46 and 66, respectively; (xiv) 48 and 66, respectively; (xv) 50 and 66, respectively; (xvi) 52 and 66, respectively; (xvii) 54 and 66, respectively; (xviii) 56 and 66, respectively; (xix) 58 and 66, respectively; (xx) 60 and 66, respectively; (xxi) 63 and 66, respectively; (xxii) 65 and 66, respectively; (xxiii) 45 and 68, respectively; (xxiv) 46 and 68, respectively; (xxv) 48 and 68, respectively; (xxvi) 50 and 68, respectively; (xxvii) 52 and 68, respectively; (xxviii) 54 and 68, respectively; (xxix) 56 and 68, respectively; (xxx) 58 and 68, respectively; (xxxi) 60 and 68, respectively; (xxxii) 63 and 68, respectively; (xxxiii) 65 and 68, respectively; (xxxiv) 45, 46, 48, 50, 52, 54, 56, 58, 60, 63, or 65, and 8, respectively; (xxxv) 45, 46, 48, 50, 52, 54, 56, 58, 60, 63, or 65, and 17, respectively; (xxxvi) 45, 46, 48, 50, 52, 54, 56, 58, 60, 63, or 65, and 20, respectively; (xxxvii) 45, 46, 48, 50, 52, 54, 56, 58, 60, 63, or 65, and 23, respectively; (xxxviii) 45, 46, 48, 50, 52, 54, 56, 58, 60, 63, or 65, and 31, respectively; (xxxix) 2, 45, 46, 48, 50, 52, 54, 56, 58, 60, 63, or 65, and 37, respectively; (xxxx) 2, 45, 46, 48, 50, 52, 54, 56, 58, 60, 63, or 65, and 66, respectively; (xxxxi) 2, 45, 46, 48, 50, 52, 54, 56, 58, 60, 63, or 65, and 68, respectively; (xxxxii) 2, 14, 45, 46, 48, 50, 52, 54, 56, 58, 60, 63, or 65, and 66, respectively; (xxxxiii) 2 and 8, respectively; or (xxxxiv) 2 and 37, respectively. In some embodiments, CDRH3 and CDRL3 are according to the IMGT definition. In some embodiments, CDRH3 and CDRL3 are according to the IMGT-junction definition.

In some embodiments, the VH and VL of an antibody or antigen-binding fragment comprise the CDRH1, CDRH2, CDRH3, CDRL1, CDRL2, and CDRL3 amino acid sequences, as determined by the Kabat numbering system or method, of the VH and VL set forth in SEQ ID NOs: (i) 45 and 37, respectively; (ii) 46 and 37, respectively; (iii) 48 and 37, respectively; (iv) 50 and 37, respectively; (v) 52 and 37, respectively; (vi) 54 and 37, respectively; (vii) 56 and 37, respectively; (viii) 58 and 37, respectively; (ix) 60 and 37, respectively; (x) 63 and 37, respectively; (xi) 65 and 37, respectively; (xii) 45 and 66, respectively; (xiii) 46 and 66, respectively; (xiv) 48 and 66, respectively; (xv) 50 and 66, respectively; (xvi) 52 and 66, respectively; (xvii) 54 and 66, respectively; (xviii) 56 and 66, respectively; (xix) 58 and 66, respectively; (xx) 60 and 66, respectively; (xxi) 63 and 66, respectively; (xxii) 65 and 66, respectively; (xxiii) 45 and 68, respectively; (xxiv) 46 and 68, respectively; (xxv) 48 and 68, respectively; (xxvi) 50 and 68, respectively; (xxvii) 52 and 68, respectively; (xxviii) 54 and 68, respectively; (xxix) 56 and 68, respectively; (xxx) 58 and 68, respectively; (xxxi) 60 and 68, respectively; (xxxii) 63 and 68, respectively; (xxxiii) 65 and 68, respectively; (xxxiv) 45, 46, 48, 50, 52, 54, 56, 58, 60, 63, or 65, and 8, respectively; (xxxv) 45, 46, 48, 50, 52, 54, 56, 58, 60, 63, or 65, and 17, respectively; (xxxvi) 45, 46, 48, 50, 52, 54, 56, 58, 60, 63, or 65, and 20, respectively; (xxxvii) 45, 46, 48, 50, 52, 54, 56, 58, 60, 63, or 65, and 23, respectively; (xxxviii) 45, 46, 48, 50, 52, 54, 56, 58, 60, 63, or 65, and 31, respectively; (xxxix) 2, 45, 46, 48, 50, 52, 54, 56, 58, 60, 63, or 65, and 37, respectively; (xxxx) 2, 45, 46, 48, 50, 52, 54, 56, 58, 60, 63, or 65, and 66, respectively; (xxxxi) 2, 45, 46, 48, 50, 52, 54, 56, 58, 60, 63, or 65, and 68, respectively; (xxxxii) 2, 14, 45, 46, 48, 50, 52, 54, 56, 58, 60, 63, or 65, and 66, respectively; (xxxxiii) 2 and 8, respectively; or (xxxxiv) 2 and 37, respectively.

In some embodiments, the VH and VL of an antibody or antigen-binding fragment comprise the CDRH1, CDRH2, CDRH3, CDRL1, CDRL2, and CDRL3 amino acid sequences, as determined by the Chothia numbering system or method, of the VH and VL set forth in SEQ ID NOs: (i) 45 and 37, respectively; (ii) 46 and 37, respectively; (iii) 48 and 37, respectively; (iv) 50 and 37, respectively; (v) 52 and 37, respectively; (vi) 54 and 37, respectively; (vii) 56 and 37, respectively; (viii) 58 and 37, respectively; (ix) 60 and 37, respectively; (x) 63 and 37, respectively; (xi) 65 and 37, respectively; (xii) 45 and 66, respectively; (xiii) 46 and 66, respectively; (xiv) 48 and 66, respectively; (xv) 50 and 66, respectively; (xvi) 52 and 66, respectively; (xvii) 54 and 66, respectively; (xviii) 56 and 66, respectively; (xix) 58 and 66, respectively; (xx) 60 and 66, respectively; (xxi) 63 and 66, respectively; (xxii) 65 and 66, respectively; (xxiii) 45 and 68, respectively; (xxiv) 46 and 68, respectively; (xxv) 48 and 68, respectively; (xxvi) 50 and 68, respectively; (xxvii) 52 and 68, respectively; (xxviii) 54 and 68, respectively; (xxix) 56 and 68, respectively; (xxx) 58 and 68, respectively; (xxxi) 60 and 68, respectively; (xxxii) 63 and 68, respectively; (xxxiii) 65 and 68, respectively; (xxxiv) 45, 46, 48, 50, 52, 54, 56, 58, 60, 63, or 65, and 8, respectively; (xxxv) 45, 46, 48, 50, 52, 54, 56, 58, 60, 63, or 65, and 17, respectively; (xxxvi) 45, 46, 48, 50, 52, 54, 56, 58, 60, 63, or 65, and 20, respectively; (xxxvii) 45, 46, 48, 50, 52, 54, 56, 58, 60, 63, or 65, and 23, respectively; (xxxviii) 45, 46, 48, 50, 52, 54, 56, 58, 60, 63, or 65, and 31, respectively; (xxxix) 2, 45, 46, 48, 50, 52, 54, 56, 58, 60, 63, or 65, and 37, respectively; (xxxx) 2, 45, 46, 48, 50, 52, 54, 56, 58, 60, 63, or 65, and 66, respectively; (xxxxi) 2, 45, 46, 48, 50, 52, 54, 56, 58, 60, 63, or 65, and 68, respectively; (xxxxii) 2, 14, 45, 46, 48, 50, 52, 54, 56, 58, 60, 63, or 65, and 66, respectively; (xxxxiii) 2 and 8, respectively; or (xxxxiv) 2 and 37, respectively.

In some embodiments, the VH and VL of an antibody or antigen-binding fragment comprise the CDRH1, CDRH2, CDRH3, CDRL1, CDRL2, and CDRL3 amino acid sequences, as determined by the Enhanced Chothia numbering system or method, of the VH and VL set forth in SEQ ID NOs: (i) 45 and 37, respectively; (ii) 46 and 37, respectively; (iii) 48 and 37, respectively; (iv) 50 and 37, respectively; (v) 52 and 37, respectively; (vi) 54 and 37, respectively; (vii) 56 and 37, respectively; (viii) 58 and 37, respectively; (ix) 60 and 37, respectively; (x) 63 and 37, respectively; (xi) 65 and 37, respectively; (xii) 45 and 66, respectively; (xiii) 46 and 66, respectively; (xiv) 48 and 66, respectively; (xv) 50 and 66, respectively; (xvi) 52 and 66, respectively; (xvii) 54 and 66, respectively; (xviii) 56 and 66, respectively; (xix) 58 and 66, respectively; (xx) 60 and 66, respectively; (xxi) 63 and 66, respectively; (xxii) 65 and 66, respectively; (xxiii) 45 and 68, respectively; (xxiv) 46 and 68, respectively; (xxv) 48 and 68, respectively; (xxvi) 50 and 68, respectively; (xxvii) 52 and 68, respectively; (xxviii) 54 and 68, respectively; (xxix) 56 and 68, respectively; (xxx) 58 and 68, respectively; (xxxi) 60 and 68, respectively; (xxxii) 63 and 68, respectively; (xxxiii) 65 and 68, respectively; (xxxiv) 45, 46, 48, 50, 52, 54, 56, 58, 60, 63, or 65, and 8, respectively; (xxxv) 45, 46, 48, 50, 52, 54, 56, 58, 60, 63, or 65, and 17, respectively; (xxxvi) 45, 46, 48, 50, 52, 54, 56, 58, 60, 63, or 65, and 20, respectively; (xxxvii) 45, 46, 48, 50, 52, 54, 56, 58, 60, 63, or 65, and 23, respectively; (xxxviii) 45, 46, 48, 50, 52, 54, 56, 58, 60, 63, or 65, and 31, respectively; (xxxix) 2, 45, 46, 48, 50, 52, 54, 56, 58, 60, 63, or 65, and 37, respectively; (xxxx) 2, 45, 46, 48, 50, 52, 54, 56, 58, 60, 63, or 65, and 66, respectively; (xxxxi) 2, 45, 46, 48, 50, 52, 54, 56, 58, 60, 63, or 65, and 68, respectively; (xxxxii) 2, 14, 45, 46, 48, 50, 52, 54, 56, 58, 60, 63, or 65, and 66, respectively; (xxxxiii) 2 and 8, respectively; or (xxxxiv) 2 and 37, respectively.

In some embodiments, the VH and VL of an antibody or antigen-binding fragment comprise the CDRH1, CDRH2, CDRH3, CDRL1, CDRL2, and CDRL3 amino acid sequences, as determined by the AHo numbering system or method, of the VH and VL set forth in SEQ ID NOs: (i) 45 and 37, respectively; (ii) 46 and 37, respectively; (iii) 48 and 37, respectively; (iv) 50 and 37, respectively; (v) 52 and 37, respectively; (vi) 54 and 37, respectively; (vii) 56 and 37, respectively; (viii) 58 and 37, respectively; (ix) 60 and 37, respectively; (x) 63 and 37, respectively; (xi) 65 and 37, respectively; (xii) 45 and 66, respectively; (xiii) 46 and 66, respectively; (xiv) 48 and 66, respectively; (xv) 50 and 66, respectively; (xvi) 52 and 66, respectively; (xvii) 54 and 66, respectively; (xviii) 56 and 66, respectively; (xix) 58 and 66, respectively; (xx) 60 and 66, respectively; (xxi) 63 and 66, respectively; (xxii) 65 and 66, respectively; (xxiii) 45 and 68, respectively; (xxiv) 46 and 68, respectively; (xxv) 48 and 68, respectively; (xxvi) 50 and 68, respectively; (xxvii) 52 and 68, respectively; (xxviii) 54 and 68, respectively; (xxix) 56 and 68, respectively; (xxx) 58 and 68, respectively; (xxxi) 60 and 68, respectively; (xxxii) 63 and 68, respectively; (xxxiii) 65 and 68, respectively; (xxxiv) 45, 46, 48, 50, 52, 54, 56, 58, 60, 63, or 65, and 8, respectively; (xxxv) 45, 46, 48, 50, 52, 54, 56, 58, 60, 63, or 65, and 17, respectively; (xxxvi) 45, 46, 48, 50, 52, 54, 56, 58, 60, 63, or 65, and 20, respectively; (xxxvii) 45, 46, 48, 50, 52, 54, 56, 58, 60, 63, or 65, and 23, respectively; (xxxviii) 45, 46, 48, 50, 52, 54, 56, 58, 60, 63, or 65, and 31, respectively; (xxxix) 2, 45, 46, 48, 50, 52, 54, 56, 58, 60, 63, or 65, and 37, respectively; (xxxx) 2, 45, 46, 48, 50, 52, 54, 56, 58, 60, 63, or 65, and 66, respectively; (xxxxi) 2, 45, 46, 48, 50, 52, 54, 56, 58, 60, 63, or 65, and 68, respectively; (xxxxii) 2, 14, 45, 46, 48, 50, 52, 54, 56, 58, 60, 63, or 65, and 66, respectively; (xxxxiii) 2 and 8, respectively; or (xxxxiv) 2 and 37, respectively.

In some embodiments, the VH and VL of an antibody or antigen-binding fragment comprise the CDRH1, CDRH2, CDRH3, CDRL1, CDRL2, and CDRL3 amino acid sequences, as determined by the North numbering system or method, of the VH and VL set forth in SEQ ID NOs: (i) 45 and 37, respectively; (ii) 46 and 37, respectively; (iii) 48 and 37, respectively; (iv) 50 and 37, respectively; (v) 52 and 37, respectively; (vi) 54 and 37, respectively; (vii) 56 and 37, respectively; (viii) 58 and 37, respectively; (ix) 60 and 37, respectively; (x) 63 and 37, respectively; (xi) 65 and 37, respectively; (xii) 45 and 66, respectively; (xiii) 46 and 66, respectively; (xiv) 48 and 66, respectively; (xv) 50 and 66, respectively; (xvi) 52 and 66, respectively; (xvii) 54 and 66, respectively; (xviii) 56 and 66, respectively; (xix) 58 and 66, respectively; (xx) 60 and 66, respectively; (xxi) 63 and 66, respectively; (xxii) 65 and 66, respectively; (xxiii) 45 and 68, respectively; (xxiv) 46 and 68, respectively; (xxv) 48 and 68, respectively; (xxvi) 50 and 68, respectively; (xxvii) 52 and 68, respectively; (xxviii) 54 and 68, respectively; (xxix) 56 and 68, respectively; (xxx) 58 and 68, respectively; (xxxi) 60 and 68, respectively; (xxxii) 63 and 68, respectively; (xxxiii) 65 and 68, respectively; (xxxiv) 45, 46, 48, 50, 52, 54, 56, 58, 60, 63, or 65, and 8, respectively; (xxxv) 45, 46, 48, 50, 52, 54, 56, 58, 60, 63, or 65, and 17, respectively; (xxxvi) 45, 46, 48, 50, 52, 54, 56, 58, 60, 63, or 65, and 20, respectively; (xxxvii) 45, 46, 48, 50, 52, 54, 56, 58, 60, 63, or 65, and 23, respectively; (xxxviii) 45, 46, 48, 50, 52, 54, 56, 58, 60, 63, or 65, and 31, respectively; (xxxix) 2, 45, 46, 48, 50, 52, 54, 56, 58, 60, 63, or 65, and 37, respectively; (xxxx) 2, 45, 46, 48, 50, 52, 54, 56, 58, 60, 63, or 65, and 66, respectively; (xxxxi) 2, 45, 46, 48, 50, 52, 54, 56, 58, 60, 63, or 65, and 68, respectively; (xxxxii) 2, 14, 45, 46, 48, 50, 52, 54, 56, 58, 60, 63, or 65, and 66, respectively; (xxxxiii) 2 and 8, respectively; or (xxxxiv) 2 and 37, respectively.

In some embodiments, the VH and VL of an antibody or antigen-binding fragment comprise the CDRH1, CDRH2, CDRH3, CDRL1, CDRL2, and CDRL3 amino acid sequences, as determined by the Contact numbering system or method, of the VH and VL set forth in SEQ ID NOs: (i) 45 and 37, respectively; (ii) 46 and 37, respectively; (iii) 48 and 37, respectively; (iv) 50 and 37, respectively; (v) 52 and 37, respectively; (vi) 54 and 37, respectively; (vii) 56 and 37, respectively; (viii) 58 and 37, respectively; (ix) 60 and 37, respectively; (x) 63 and 37, respectively; (xi) 65 and 37, respectively; (xii) 45 and 66, respectively; (xiii) 46 and 66, respectively; (xiv) 48 and 66, respectively; (xv) 50 and 66, respectively; (xvi) 52 and 66, respectively; (xvii) 54 and 66, respectively; (xviii) 56 and 66, respectively; (xix) 58 and 66, respectively; (xx) 60 and 66, respectively; (xxi) 63 and 66, respectively; (xxii) 65 and 66, respectively; (xxiii) 45 and 68, respectively; (xxiv) 46 and 68, respectively; (xxv) 48 and 68, respectively; (xxvi) 50 and 68, respectively; (xxvii) 52 and 68, respectively; (xxviii) 54 and 68, respectively; (xxix) 56 and 68, respectively; (xxx) 58 and 68, respectively; (xxxi) 60 and 68, respectively; (xxxii) 63 and 68, respectively; (xxxiii) 65 and 68, respectively; (xxxiv) 45, 46, 48, 50, 52, 54, 56, 58, 60, 63, or 65, and 8, respectively; (xxxv) 45, 46, 48, 50, 52, 54, 56, 58, 60, 63, or 65, and 17, respectively; (xxxvi) 45, 46, 48, 50, 52, 54, 56, 58, 60, 63, or 65, and 20, respectively; (xxxvii) 45, 46, 48, 50, 52, 54, 56, 58, 60, 63, or 65, and 23, respectively; (xxxviii) 45, 46, 48, 50, 52, 54, 56, 58, 60, 63, or 65, and 31, respectively; (xxxix) 2, 45, 46, 48, 50, 52, 54, 56, 58, 60, 63, or 65, and 37, respectively; (xxxx) 2, 45, 46, 48, 50, 52, 54, 56, 58, 60, 63, or 65, and 66, respectively; (xxxxi) 2, 45, 46, 48, 50, 52, 54, 56, 58, 60, 63, or 65, and 68, respectively; (xxxxii) 2, 14, 45, 46, 48, 50, 52, 54, 56, 58, 60, 63, or 65, and 66, respectively; (xxxxiii) 2 and 8, respectively; or (xxxxiv) 2 and 37, respectively.

In some embodiments, the VH and VL of an antibody or antigen-binding fragment comprise the CDRH1, CDRH2, CDRH3, CDRL1, CDRL2, and CDRL3 amino acid sequences, as determined by the Martin numbering system or method, of the VH and VL set forth in SEQ ID NOs: (i) 45 and 37, respectively; (ii) 46 and 37, respectively; (iii) 48 and 37, respectively; (iv) 50 and 37, respectively; (v) 52 and 37, respectively; (vi) 54 and 37, respectively; (vii) 56 and 37, respectively; (viii) 58 and 37, respectively; (ix) 60 and 37, respectively; (x) 63 and 37, respectively; (xi) 65 and 37, respectively; (xii) 45 and 66, respectively; (xiii) 46 and 66, respectively; (xiv) 48 and 66, respectively; (xv) 50 and 66, respectively; (xvi) 52 and 66, respectively; (xvii) 54 and 66, respectively; (xviii) 56 and 66, respectively; (xix) 58 and 66, respectively; (xx) 60 and 66, respectively; (xxi) 63 and 66, respectively; (xxii) 65 and 66, respectively; (xxiii) 45 and 68, respectively; (xxiv) 46 and 68, respectively; (xxv) 48 and 68, respectively; (xxvi) 50 and 68, respectively; (xxvii) 52 and 68, respectively; (xxviii) 54 and 68, respectively; (xxix) 56 and 68, respectively; (xxx) 58 and 68, respectively; (xxxi) 60 and 68, respectively; (xxxii) 63 and 68, respectively; (xxxiii) 65 and 68, respectively; (xxxiv) 45, 46, 48, 50, 52, 54, 56, 58, 60, 63, or 65, and 8, respectively; (xxxv) 45, 46, 48, 50, 52, 54, 56, 58, 60, 63, or 65, and 17, respectively; (xxxvi) 45, 46, 48, 50, 52, 54, 56, 58, 60, 63, or 65, and 20, respectively; (xxxvii) 45, 46, 48, 50, 52, 54, 56, 58, 60, 63, or 65, and 23, respectively; (xxxviii) 45, 46, 48, 50, 52, 54, 56, 58, 60, 63, or 65, and 31, respectively; (xxxix) 2, 45, 46, 48, 50, 52, 54, 56, 58, 60, 63, or 65, and 37, respectively; (xxxx) 2, 45, 46, 48, 50, 52, 54, 56, 58, 60, 63, or 65, and 66, respectively; (xxxxi) 2, 45, 46, 48, 50, 52, 54, 56, 58, 60, 63, or 65, and 68, respectively; (xxxxii) 2, 14, 45, 46, 48, 50, 52, 54, 56, 58, 60, 63, or 65, and 66, respectively; (xxxxiii) 2 and 8, respectively; or (xxxxiv) 2 and 37, respectively.

In some embodiments, an anti-influenza neuraminidase (anti-NA) antibody, or an antigen-binding fragment thereof is provided that comprises (i) a heavy chain variable domain (VH) comprising a complementarity determining region (CDR)H1, a CDRH2, and a CDRH3, and (ii) and a light chain variable domain (VL) comprising a CDRL1, a CDRL2, and a CDRL3, wherein: (a) the CDRH1 comprises or consists of the amino acid sequence set forth in SEQ ID NO:3, SEQ ID NO:47, SEQ ID NO:49, or SEQ ID NO:55; and/or (b) the CDRH2 comprises or consists of the amino acid sequence set forth in SEQ ID NO.4, SEQ ID NO:57, or SEQ ID NO:61; and/or (c) the CDRH3 comprises or consists of the amino acid sequence set forth in SEQ ID NO:5, SEQ ID NO:15, SEQ ID NO:51, or SEQ ID NO:53, (d) the CDRL1 comprises or consists of the amino acid sequence set forth in SEQ ID NO.9 or SEQ ID NO:32; and/or (e) the CDRL2 comprises or consists of the amino acid sequence set forth in SEQ ID NO.: 10; and/or (f) the CDRL3 comprises or consists of the amino acid sequence set forth in SEQ ID NO:11, 18, 21, 24, 33, or 67, optionally provided that the CDRH1, the CDRH2, the CDRH3, the CDRL1, the CDRL2, and the CDRL3 do not comprise or consist of the amino acid sequences set forth in SEQ ID NOs: (i) 3-5 and 9-11, respectively; (ii) 3, 4, 15 and 9-11, respectively; (iii) 3-5, 9, 10, and 18, respectively; (iv) 3-5, 9, 10, and 21, respectively; (v) 3-5, 9-11, and 24, respectively; or (vi) 3-5, 32, 96, and 33, respectively.

In certain embodiments, the CDRH3 and CDRL3 comprise or consist of the amino acid sequences set forth in: (i) 5 and 11, respectively; (ii) 5 and 18, respectively; (iii) 5 and 21, respectively; (iv) 5 and 24, respectively; (v) 5 and 33, respectively; (vi) 5 and 67, respectively; (vii) 15 and 11, respectively; (viii) 15 and 18, respectively; (ix) 15 and 21, respectively; (x) 15 and 24, respectively; (xi) 15 and 33, respectively; (xii) 15 and 67, respectively; (xiii) 51 and 11, respectively; (xiv) 51 and 18, respectively; (xv) 51 and 21, respectively; (xvi) 51 and 24, respectively; (xvii) 51 and 33, respectively; (xviii) 51 and 67, respectively; (xix) 53 and 11, respectively; (xx) 53 and 18, respectively; (xxi) 53 and 21, respectively; (xxii) 53 and 24, respectively; (xxiii) 53 and 33, respectively; or (xxiv) 53 and 67, respectively.

In certain further embodiments, the CDRH1, CDRH2, CDRH3, CDRL1, CDRL2, and CDRL3 comprise or consist of the amino acid sequences set forth in SEQ ID NOs: (i) 3-5, 9, 10, and 67, respectively; (ii) 47, 4, 5, and 9-11, respectively; (iii) 47, 4, 5, 9, 10, and 67, respectively; (iv) 49, 4, 5, and 9-11, respectively; (v) 47, 4, 5, 9, 10, and 67, respectively; (vi) 55, 4, 5, and 9-11, respectively; (vii) 55, 4, 5, 9, 10, and 67, respectively; (viii) 3, 4, 51, and 9-11, respectively; (ix) 3, 4, 51, 9, 10, and 67, respectively; (x) 55, 4, 5, and 9-11, respectively; (xi) 55, 4, 5, 9, 10, and 67, respectively; (xii) 3, 61, 5, and 9-11, respectively; (xiii) 3, 61, 5, 9, 10, and 67 respectively; or (xiv) 3-5 and 9-11, respectively.

In some embodiments, an anti-influenza neuraminidase (anti-NA) antibody, or an antigen-binding fragment thereof is provided that comprises (i) a heavy chain variable domain (VH) and (ii) light chain variable domain (VL), wherein: (i) the VH comprises the complementarity determining region (CDR)H1, CDRH2, and CDRH3 of the VH amino acid sequence set forth in SEQ ID NO:2, and the VL comprises the CDRL1, CDRL2, and CDRL3 of the VL amino acid sequence set forth in SEQ ID NO:66; (ii) the VH comprises the CDRH1, CDRH2, and CDRH3 of the VH amino acid sequence set forth in SEQ ID NO:46, and the VL comprises the CDRL1, CDRL2, and CDRL3 of the VL amino acid sequence set forth in SEQ ID NO:8; (iii) the VH comprises the CDRH1, CDRH2, and CDRH3 of the VH amino acid sequence set forth in SEQ ID NO:46, and the VL comprises the CDRL1, CDRL2, and CDRL3 of the VL amino acid sequence set forth in SEQ ID NO.: 17; (iv) the VH comprises the CDRH1, CDRH2, and CDRH3 of the VH amino acid sequence set forth in SEQ ID NO:46, and the VL comprises the CDRL1, CDRL2, and CDRL3 of the VL amino acid sequence set forth in SEQ ID NO:20; (v) the VH comprises the CDRH1, CDRH2, and CDRH3 of the VH amino acid sequence set forth in SEQ ID NO:46, and the VL comprises the CDRL1, CDRL2, and CDRL3 of the VL amino acid sequence set forth in SEQ ID NO:23; (vi) the VH comprises the CDRH1, CDRH2, and CDRH3 of the VH amino acid sequence set forth in SEQ ID NO:46, and the VL comprises the CDRL1, CDRL2, and CDRL3 of the VL amino acid sequence set forth in SEQ ID NO:31; (vii) the VH comprises the CDRH1, CDRH2, and CDRH3 of the VH amino acid sequence set forth in SEQ ID NO:46, and the VL comprises the CDRL1, CDRL2, and CDRL3 of the VL amino acid sequence set forth in SEQ ID NO:66; (viii) the VH comprises the CDRH1, CDRH2, and CDRH3 of the VH amino acid sequence set forth in SEQ ID NO:48, and the VL comprises the CDRL1, CDRL2, and CDRL3 of the VL amino acid sequence set forth in SEQ ID NO:8; (ix) the VH comprises the CDRH1, CDRH2, and CDRH3 of the VH amino acid sequence set forth in SEQ ID NO:48, and the VL comprises the CDRL1, CDRL2, and CDRL3 of the VL amino acid sequence set forth in SEQ ID NO:17; (x) the VH comprises the CDRH1, CDRH2, and CDRH3 of the VH amino acid sequence set forth in SEQ ID NO:48, and the VL comprises the CDRL1, CDRL2, and CDRL3 of the VL amino acid sequence set forth in SEQ ID NO:20; (xi) the VH comprises the CDRH1, CDRH2, and CDRH3 of the VH amino acid sequence set forth in SEQ ID NO:48, and the VL comprises the CDRL1, CDRL2, and CDRL3 of the VL amino acid sequence set forth in SEQ ID NO:23; (xii) the VH comprises the CDRH1, CDRH2, and CDRH3 of the VH amino acid sequence set forth in SEQ ID NO:48, and the VL comprises the CDRL1, CDRL2, and CDRL3 of the VL amino acid sequence set forth in SEQ ID NO:31; (xiii) the VH comprises the CDRH1, CDRH2, and CDRH3 of the VH amino acid sequence set forth in SEQ ID NO:48, and the VL comprises the CDRL1, CDRL2, and CDRL3 of the VL amino acid sequence set forth in SEQ ID NO:66; (xiv) the VH comprises the CDRH1, CDRH2, and CDRH3 of the VH amino acid sequence set forth in SEQ ID NO:54, and the VL comprises the CDRL1, CDRL2, and CDRL3 of the VL amino acid sequence set forth in SEQ ID NO:8; (xv) the VH comprises the CDRH1, CDRH2, and CDRH3 of the VH amino acid sequence set forth in SEQ ID NO:54, and the VL comprises the CDRL1, CDRL2, and CDRL3 of the VL amino acid sequence set forth in SEQ ID NO:17; (xvi) the VH comprises the CDRH1, CDRH2, and CDRH3 of the VH amino acid sequence set forth in SEQ ID NO:54, and the VL comprises the CDRL1, CDRL2, and CDRL3 of the VL amino acid sequence set forth in SEQ ID NO:20; (xvii) the VH comprises the CDRH1, CDRH2, and CDRH3 of the VH amino acid sequence set forth in SEQ ID NO:54, and the VL comprises the CDRL1, CDRL2, and CDRL3 of the VL amino acid sequence set forth in SEQ ID NO:23; (xviii) the VH comprises the CDRH1, CDRH2, and CDRH3 of the VH amino acid sequence set forth in SEQ ID NO:54, and the VL comprises the CDRL1, CDRL2, and CDRL3 of the VL amino acid sequence set forth in SEQ ID NO:31; (xix) the VH comprises the CDRH1, CDRH2, and CDRH3 of the VH amino acid sequence set forth in SEQ ID NO:54, and the VL comprises the CDRL1, CDRL2, and CDRL3 of the VL amino acid sequence set forth in SEQ ID NO:66; (xx) the VH comprises the CDRH1, CDRH2, and CDRH3 of the VH amino acid sequence set forth in SEQ ID NO:56, and the VL comprises the CDRL1, CDRL2, and CDRL3 of the VL amino acid sequence set forth in SEQ ID NO:8; (xxi) the VH comprises the CDRH1, CDRH2, and CDRH3 of the VH amino acid sequence set forth in SEQ ID NO:56, and the VL comprises the CDRL1, CDRL2, and CDRL3 of the VL amino acid sequence set forth in SEQ ID NO:17; (xxii) the VH comprises the CDRH1, CDRH2, and CDRH3 of the VH amino acid sequence set forth in SEQ ID NO:56, and the VL comprises the CDRL1, CDRL2, and CDRL3 of the VL amino acid sequence set forth in SEQ ID NO:20; (xxiii) the VH comprises the CDRH1, CDRH2, and CDRH3 of the VH amino acid sequence set forth in SEQ ID NO:56, and the VL comprises the CDRL1, CDRL2, and CDRL3 of the VL amino acid sequence set forth in SEQ ID NO:23; (xxiv) the VH comprises the CDRH1, CDRH2, and CDRH3 of the VH amino acid sequence set forth in SEQ ID NO:54, and the VL comprises the CDRL1, CDRL2, and CDRL3 of the VL amino acid sequence set forth in SEQ ID NO:31; (xxv) the VH comprises the CDRH1, CDRH2, and CDRH3 of the VH amino acid sequence set forth in SEQ ID NO:54, and the VL comprises the CDRL1, CDRL2, and CDRL3 of the VL amino acid sequence set forth in SEQ ID NO:66; (xxvi) the VH comprises the CDRH1, CDRH2, and CDRH3 of the VH amino acid sequence set forth in SEQ ID NO:60, and the VL comprises the CDRL1, CDRL2, and CDRL3 of the VL amino acid sequence set forth in SEQ ID NO:8; (xxvii) the VH comprises the CDRH1, CDRH2, and CDRH3 of the VH amino acid sequence set forth in SEQ ID NO:60, and the VL comprises the CDRL1, CDRL2, and CDRL3 of the VL amino acid sequence set forth in SEQ ID NO:17; (xxviii) the VH comprises the CDRH1, CDRH2, and CDRH3 of the VH amino acid sequence set forth in SEQ ID NO:60, and the VL comprises the CDRL1, CDRL2, and CDRL3 of the VL amino acid sequence set forth in SEQ ID NO:20; (xxix) the VH comprises the CDRH1, CDRH2, and CDRH3 of the VH amino acid sequence set forth in SEQ ID NO:60, and the VL comprises the CDRL1, CDRL2, and CDRL3 of the VL amino acid sequence set forth in SEQ ID NO:23; (xxx) the VH comprises the CDRH1, CDRH2, and CDRH3 of the VH amino acid sequence set forth in SEQ ID NO:60, and the VL comprises the CDRL1, CDRL2, and CDRL3 of the VL amino acid sequence set forth in SEQ ID NO:31; (xxxi) the VH comprises the CDRH1, CDRH2, and CDRH3 of the VH amino acid sequence set forth in SEQ ID NO:60, and the VL comprises the CDRL1, CDRL2, and CDRL3 of the VL amino acid sequence set forth in SEQ ID NO:66; (xxxii) the VH comprises the CDRH1, CDRH2, and CDRH3 of the VH amino acid sequence set forth in SEQ ID NO:50, and the VL comprises the CDRL1, CDRL2, and CDRL3 of the VL amino acid sequence set forth in SEQ ID NO:8; (xxxiii) the VH comprises the CDRH1, CDRH2, and CDRH3 of the VH amino acid sequence set forth in SEQ ID NO:50, and the VL comprises the CDRL1, CDRL2, and CDRL3 of the VL amino acid sequence set forth in SEQ ID NO:17; (xxxiv) the VH comprises the CDRH1, CDRH2, and CDRH3 of the VH amino acid sequence set forth in SEQ ID NO:50, and the VL comprises the CDRL1, CDRL2, and CDRL3 of the VL amino acid sequence set forth in SEQ ID NO:20; (xxxv) the VH comprises the CDRH1, CDRH2, and CDRH3 of the VH amino acid sequence set forth in SEQ ID NO:50, and the VL comprises the CDRL1, CDRL2, and CDRL3 of the VL amino acid sequence set forth in SEQ ID NO:23; (xxxvi) the VH comprises the CDRH1, CDRH2, and CDRH3 of the VH amino acid sequence set forth in SEQ ID NO:50, and the VL comprises the CDRL1, CDRL2, and CDRL3 of the VL amino acid sequence set forth in SEQ ID NO:31; (xxxvii) the VH comprises the CDRH1, CDRH2, and CDRH3 of the VH amino acid sequence set forth in SEQ ID NO:50, and the VL comprises the CDRL1, CDRL2, and CDRL3 of the VL amino acid sequence set forth in SEQ ID NO:66; (xxxviii) the VH comprises the CDRH1, CDRH2, and CDRH3 of the VH amino acid sequence set forth in SEQ ID NO:52, and the VL comprises the CDRL1, CDRL2, and CDRL3 of the VL amino acid sequence set forth in SEQ ID NO:8; (xxxix) the VH comprises the CDRH1, CDRH2, and CDRH3 of the VH amino acid sequence set forth in SEQ ID NO:52, and the VL comprises the CDRL1, CDRL2, and CDRL3 of the VL amino acid sequence set forth in SEQ ID NO:17; (xxxx) the VH comprises the CDRH1, CDRH2, and CDRH3 of the VH amino acid sequence set forth in SEQ ID NO:52, and the VL comprises the CDRL1, CDRL2, and CDRL3 of the VL amino acid sequence set forth in SEQ ID NO:20; (xxxxi) the VH comprises the CDRH1, CDRH2, and CDRH3 of the VH amino acid sequence set forth in SEQ ID NO:52, and the VL comprises the CDRL1, CDRL2, and CDRL3 of the VL amino acid sequence set forth in SEQ ID NO:23; (xxxxii) the VH comprises the CDRH1, CDRH2, and CDRH3 of the VH amino acid sequence set forth in SEQ ID NO:52, and the VL comprises the CDRL1, CDRL2, and CDRL3 of the VL amino acid sequence set forth in SEQ ID NO:31; (xxxxiii) the VH comprises the CDRH1, CDRH2, and CDRH3 of the VH amino acid sequence set forth in SEQ ID NO:52, and the VL comprises the CDRL1, CDRL2, and CDRL3 of the VL amino acid sequence set forth in SEQ ID NO:66; or (xxxxiv) the VH comprises the CDRH1, CDRH2, and CDRH3 of the VH amino acid sequence set forth in SEQ ID NO:2, and the VL comprises the CDRL1, CDRL2, and CDRL3 of the VL amino acid sequence set forth in SEQ ID NO:8, wherein CDRH1, CDRH2, CDRH3, CDRL1, CDRL2, and CDRL3 are optionally defined in accordance with the IMGT numbering system.

In certain embodiments, (i) the VH comprises or consists of amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity (or similarity) to, or comprises or consists of, the amino acid sequence set forth in any one of SEQ ID NOs:2, 14, 45, 46, 48, 50, 52, 54, 56, 58, 60, 63, and 65; and (ii) the VL comprises or consists of amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity (or similarity) to, or comprises or consists of, the amino acid sequence set forth in any one of SEQ ID NOs:8, 17, 20, 23, 31, 37, 66, and 68, optionally provided that the VH and VL do not comprise or consist of the amino acid sequences set forth in: (a) 2 and 8, respectively; (b) 14 and 8, respectively; (c) 2 and 17, respectively; (d) 2 and 20, respectively; (e) 2 and 23, respectively; or (f) 2 and 31, respectively. In some embodiments, (i) the VH comprises or consists of amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity (or similarity) to, or comprises or consists of, the amino acid sequence set forth in SEQ ID NO:54, and (ii) the VL comprises or consists of amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity (or similarity) to, or comprises or consists of, the amino acid sequence set forth in SEQ ID NO.: 8.

In some embodiments, the VH and VL have at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity (or similarity) to, or comprise or consist of, the amino acid sequences set forth in SEQ ID NOs: (i) 2 and 66, respectively; (ii) 46 and 8, respectively; (iii) 46 and 17, respectively; (iv) 46 and 20, respectively; (v) 46 and 23, respectively; (vi) 46 and 31, respectively; (vii) 46 and 66, respectively; (viii) 48 and 8, respectively; (ix) 48 and 17, respectively; (x) 48 and 20, respectively; (xi) 48 and 23, respectively; (xii) 48 and 31, respectively; (xiii) 48 and 66, respectively; (xiv) 54 and 8, respectively; (xv) 54 and 17, respectively; (xvi) 54 and 20, respectively; (xvii) 54 and 23, respectively; (xviii) 54 and 31; (xix) 54 and 66, respectively; (xx) 56 and 8, respectively; (xxi) 56 and 17, respectively; (xxii) 56 and 20, respectively; (xxiii) 56 and 23, respectively; (xxiv) 54 and 31, respectively; (xxv) 54 and 66, respectively; (xxvi) 60 and 8, respectively; (xxvii) 60 and 17, respectively; (xxviii) 60 and 20, respectively; (xxix) 60 and 23, respectively; (xxx) 60 and 31, respectively; (xxxi) 60 and 66, respectively; (xxxii) 2, 14, 45, 46, 48, 50, 52, 54, 56, 58, 60, 63, or 65, and 37, respectively; (xxxiii) 2, 14, 45, 46, 48, 50, 52, 54, 56, 58, 60, 63, or 65, and 68, respectively; (xxxiv) 45, 46, 48, 50, 52, 54, 56, 58, 60, 63, or 65, and 8, respectively; (xxxv) 45, 46, 48, 50, 52, 54, 56, 58, 60, 63, or 65, and 17, respectively; (xxxvi) 45, 46, 48, 50, 52, 54, 56, 58, 60, 63, or 65, and 20, respectively; (xxxvii) 45, 46, 48, 50, 52, 54, 56, 58, 60, 63, or 65, and 23, respectively; (xxxviii) 45, 46, 48, 50, 52, 54, 56, 58, 60, 63, or 65, and 31, respectively; (xxxix) 45, 46, 48, 50, 52, 54, 56, 58, 60, 63, or 65, and 37, respectively; (xxxx) 45, 46, 48, 50, 52, 54, 56, 58, 60, 63, or 65, and 66, respectively; (xxxxi) 45, 46, 48, 50, 52, 54, 56, 58, 60, 63, or 65, and 68, respectively; (xxxxii) 2, 14, 45, 46, 48, 50, 52, 54, 56, 58, 60, 63, or 65, and 66, respectively; (xxxxiii) 2 and 8, respectively; or (xxxxiv) 2 and 37, respectively.

Variable domains of antibodies and antigen-binding fragments can comprise framework region amino acid sequences. Framework region amino acid sequences can be identified using, for example, the IMGT, Kabat, Chothia, Enhanced Chothia, Contact, Martin, or AHo numbering system or method. In some embodiments, a VH comprises a framework region (FR)1, FR2, FR3, and/or FR4 of a presently disclosed VH amino acid sequence, or comprises a variant of a disclosed FR1, FR2, FR3, and/or FR4 amino acid sequence, wherein the variant comprises, consists essentially of, or consists of, one, two, three, four, five, six, seven, eight, nine, or ten amino acid substitutions, insertions, and/or deletions as compared to a disclosed VH amino acid sequence. In some embodiments, a variant of a disclosed FR1, FR2, FR3, and/or FR4 amino acid sequence has at least 80%, at least 85%, at least 90%, at least 91%, at least 92%, at least 93%, at least 94%, at least 95%, at least 96%, at least 97%, at least 98%, or at least 99% identity (or similarity) to the disclosed FR1, FR2, FR3, and/or FR4 amino acid sequence, respectively. In some embodiments, a VL comprises a framework region (FR)1, FR2, FR3, and/or FR4 of a presently disclosed VL amino acid sequence, or comprises a variant of a disclosed FR1, FR2, FR3, and/or FR4 amino acid sequence, wherein the variant comprises, consists essentially of, or consists of, one, two, three, four, five, six, seven, eight, nine, or ten amino acid substitutions, insertions, and/or deletions as compared to a disclosed VL amino acid sequence. In some embodiments, a variant of a disclosed FR1, FR2, FR3, and/or FR4 amino acid sequence has at least 80%, at least 85%, at least 90%, at least 91%, at least 92%, at least 93%, at least 94%, at least 95%, at least 96%, at least 97%, at least 98%, or at least 99% identity (or similarity) to the disclosed FR1, FR2, FR3, and/or FR4 amino acid sequence, respectively.

In some embodiments, an antibody or antigen-binding fragment comprises:
(1) a VH comprising: (i) a FR1 comprising, consisting essentially of, or consisting of an VH FR1 amino acid sequence of any one of “FNI9-VH-WT”, “FNI9-VH-FR124GL”, “FNI9-VH.4”, “FNI9-VH.5”, “FNI9-VH.6”, “FNI9-VH.7”, “FNI9-VH.8”, “FNI9-VH.9”, “FNI9-VH.10”, “FNI9-VH.11”, “FNI9-VH.12”, and “FNI9-VH.13”, as shown in FIG. 71, or a variant thereof comprising one, two, three, four, five, six, seven, eight, nine, or ten amino acid substitutions, insertions, and/or deletions, (ii) a FR2 comprising, consisting essentially of, or consisting of a VH FR2 amino acid sequence of any one of “FNI9-VH-WT”, “FNI9-VH-FR124GL”, “FNI9-VH.4”, “FNI9-VH.5”, “FNI9-VH.6”, “FNI9-VH.7”, “FNI9-VH.8”, “FNI9-VH.9”, “FNI9-VH.10”, “FNI9-VH.11”, “FNI9-VH.12”, and “FNI9-VH.13”, as shown in FIG. 71, or a variant thereof comprising one, two, three, four, five, six, seven, eight, nine, or ten amino acid substitutions, insertions, and/or deletions, (iii) a FR3 comprising, consisting essentially of, or consisting of a VH FR3 amino acid sequence of any one of “FNI9-VH-WT”, “FNI9-VH-FR124GL”, “FNI9-VH.4”, “FNI9-VH.5”, “FNI9-VH.6”, “FNI9-VH.7”, “FNI9-VH.8”, “FNI9-VH.9”, “FNI9-VH.10”, “FNI9-VH.11”, “FNI9-VH.12”, and “FNI9-VH.13”, as shown in FIG. 71, or a variant thereof comprising one, two, three, four, five, six, seven, eight, nine, or ten amino acid substitutions, insertions, and/or deletions, and/or (iv) a FR4 comprising, consisting essentially of, or consisting of a VH FR4 amino acid sequence of any one of “FNI9-VH-WT”, “FNI9-VH-FR124GL”, “FNI9-VH.4”, “FNI9-VH.5”, “FNI9-VH.6”, “FNI9-VH.7”, “FNI9-VH.8”, “FNI9-VH.9”, “FNI9-VH.10”, “FNI9-VH.11”, “FNI9-VH.12”, and “FNI9-VH.13”, as shown in FIG. 71 or a variant thereof comprising one, two, three, four, five, six, seven, eight, nine, or ten amino acid substitutions, insertions, and/or deletions; and/or
(2) a VL comprising (i) a FR1 comprising, consisting essentially of, or consisting of an VL FR1 amino acid sequence of any one of “FNI9-VK-WT”, “FNI9-VK.7”, and “FNI9-VK.8”, as shown in FIG. 71, or a variant thereof comprising one, two, three, four, five, six, seven, eight, nine, or ten amino acid substitutions, insertions, and/or deletions, (ii) a FR2 comprising, consisting essentially of, or consisting of a VL FR2 amino acid sequence of any one of “FNI9-VK-WT”, “FNI9-VK.7”, and “FNI9-VK.8”, as shown in FIG. 71, or a variant thereof comprising one, two, three, four, five, six, seven, eight, nine, or ten amino acid substitutions, insertions, and/or deletions, (iii) a FR3 comprising, consisting essentially of, or consisting of a VL FR3 amino acid sequence of any one of “FNI9-VK-WT”, “FNI9-VK.7”, and “FNI9-VK.8”, as shown in FIG. 71, or a variant thereof comprising one, two, three, four, five, six, seven, eight, nine, or ten amino acid substitutions, insertions, and/or deletions, or a variant thereof comprising one, two, three, four, five, six, seven, eight, nine, or ten amino acid substitutions, insertions, and/or deletions, and/or (iv) a FR4 comprising, consisting essentially of, or consisting of a VL FR4 amino acid sequence of any one of “FNI9-VK-WT”, “FNI9-VK.7”, and “FNI9-VK.8”, as shown in FIG. 71 or a variant thereof comprising one, two, three, four, five, six, seven, eight, nine, or ten amino acid substitutions, insertions, and/or deletions. In some embodiments, VH FR1, VH FR2, VH FR3, VH FR4, VL FR1, VL FR2, VL FR3, and VL FR4 are according to IMGT. In some embodiments, VH FR1, VH FR2, VH FR3, VH FR4, VL FR1, VL FR2, VL FR3, and VL FR4 are according to Kabat. In some embodiments, VH FR1, VH FR2, VH FR3, VH FR4, VL FR1, VL FR2, VL FR3, and VL FR4 are according to Chothia. In some embodiments, VH FR1, VH FR2, VH FR3, VH FR4, VL FR1, VL FR2, VL FR3, and VL FR4 are according to Enhanced Chothia. In some embodiments, VH FR1, VH FR2, VH FR3, VH FR4, VL FR1, VL FR2, VL FR3, and VL FR4 are according to Contact. In some embodiments, VH FR1, VH FR2, VH FR3, VH FR4, VL FR1, VL FR2, VL FR3, and VL FR4 are according to Martin. In some embodiments, VH FR1, VH FR2, VH FR3, VH FR4, VL FR1, VL FR2, VL FR3, and VL FR4 are according to North. In some embodiments, VH FR1, VH FR2, VH FR3, VH FR4, VL FR1, VL FR2, VL FR3, and VL FR4 are according to AHo.

In some embodiments, an antibody or antigen-binding fragment comprises:
(1) a VH comprising: (i) a FR1 comprising, consisting essentially of, or consisting of an amino acid sequence having at least 80%, at least 85%, at least 90%, at least 91%, at least 92%, at least 93%, at least 94%, at least 95%, at least 96%, at least 97%, at least 98%, or at least 99% identity (or similarity) to the FR1 amino acid sequence of any one of “FNI9-VH-WT”, “FNI9-VH-FR124GL”, “FNI9-VH.4”, “FNI9-VH.5”, “FNI9-VH.6”, “FNI9-VH.7”, “FNI9-VH.8”, “FNI9-VH.9”, “FNI9-VH.10”, “FNI9-VH.11”, “FNI9-VH.12”, and “FNI9-VH.13”, as shown in FIG. 71, (ii) a FR2 comprising, consisting essentially of, or consisting of an amino acid sequence having at least 80%, at least 85%, at least 90%, at least 91%, at least 92%, at least 93%, at least 94%, at least 95%, at least 96%, at least 97%, at least 98%, or at least 99% identity (or similarity) to the VH FR2 amino acid sequence of any one of “FNI9-VH-WT”, “FNI9-VH-FR124GL”, “FNI9-VH.4”, “FNI9-VH.5”, “FNI9-VH.6”, “FNI9-VH.7”, “FNI9-VH.8”, “FNI9-VH.9”, “FNI9-VH.10”, “FNI9-VH.11”, “FNI9-VH.12”, and “FNI9-VH.13”, as shown in FIG. 71, (iii) a FR3 comprising, consisting essentially of, or consisting of an amino acid sequence having at least 80%, at least 85%, at least 90%, at least 91%, at least 92%, at least 93%, at least 94%, at least 95%, at least 96%, at least 97%, at least 98%, or at least 99% identity (or similarity) to the VH FR3 amino acid sequence of any one of “FNI9-VH-WT”, “FNI9-VH-FR124GL”, “FNI9-VH.4”, “FNI9-VH.5”, “FNI9-VH.6”, “FNI9-VH.7”, “FNI9-VH.8”, “FNI9-VH.9”, “FNI9-VH.10”, “FNI9-VH.11”, “FNI9-VH.12”, and “FNI9-VH.13”, as shown in FIG. 71, and/or (iv) a FR4 comprising, consisting essentially of, or consisting of an amino acid sequence having at least 80%, at least 85%, at least 90%, at least 91%, at least 92%, at least 93%, at least 94%, at least 95%, at least 96%, at least 97%, at least 98%, or at least 99% identity (or similarity) to the VH FR4 amino acid sequence of any one of “FNI9-VH-WT”, “FNI9-VH-FR124GL”, “FNI9-VH.4”, “FNI9-VH.5”, “FNI9-VH.6”, “FNI9-VH.7”, “FNI9-VH.8”, “FNI9-VH.9”, “FNI9-VH.10”, “FNI9-VH.11”, “FNI9-VH.12”, and “FNI9-VH.13”, as shown in FIG. 71; and/or
(2) a VL comprising (i) a FR1 comprising, consisting essentially of, or consisting of an amino acid sequence having at least 80%, at least 85%, at least 90%, at least 91%, at least 92%, at least 93%, at least 94%, at least 95%, at least 96%, at least 97%, at least 98%, or at least 99% identity (or similarity) to the VL FR1 amino acid sequence of any one of “FNI9-VK-WT”, “FNI9-VK.7”, and “FNI9-VK.8”, as shown in FIG. 71, (ii) a FR2 comprising, consisting essentially of, or consisting of an amino acid sequence having at least 80%, at least 85%, at least 90%, at least 91%, at least 92%, at least 93%, at least 94%, at least 95%, at least 96%, at least 97%, at least 98%, or at least 99% identity (or similarity) to the VL FR2 amino acid sequence of any one of “FNI9-VK-WT”, “FNI9-VK.7”, and “FNI9-VK.8”, as shown in FIG. 71, (iii) a FR3 comprising, consisting essentially of, or consisting of an amino acid sequence having at least 80%, at least 85%, at least 90%, at least 91%, at least 92%, at least 93%, at least 94%, at least 95%, at least 96%, at least 97%, at least 98%, or at least 99% identity (or similarity) to the VL FR3 amino acid sequence of any one of “FNI9-VK-WT”, “FNI9-VK.7”, and “FNI9-VK.8”, as shown in FIG. 71 and/or (iv) a FR4 comprising, consisting essentially of, or consisting of an amino acid sequence having at least 80%, at least 85%, at least 90%, at least 91%, at least 92%, at least 93%, at least 94%, at least 95%, at least 96%, at least 97%, at least 98%, or at least 99% identity (or similarity) to the VL FR4 amino acid sequence of any one of “FNI9-VK-WT”, “FNI9-VK.7”, and “FNI9-VK.8”, as shown in FIG. 71. In some embodiments, VH FR1, VH FR2, VH FR3, VH FR4, VL FR1, VL FR2, VL FR3, and VL FR4 are according to IMGT. In some embodiments, VH FR1, VH FR2, VH FR3, VH FR4, VL FR1, VL FR2, VL FR3, and VL FR4 are according to Kabat. In some embodiments, VH FR1, VH FR2, VH FR3, VH FR4, VL FR1, VL FR2, VL FR3, and VL FR4 are according to Chothia. In some embodiments, VH FR1, VH FR2, VH FR3, VH FR4, VL FR1, VL FR2, VL FR3, and VL FR4 are according to Enhanced Chothia. In some embodiments, VH FR1, VH FR2, VH FR3, VH FR4, VL FR1, VL FR2, VL FR3, and VL FR4 are according to Contact. In some embodiments, VH FR1, VH FR2, VH FR3, VH FR4, VL FR1, VL FR2, VL FR3, and VL FR4 are according to Martin. In some embodiments, VH FR1, VH FR2, VH FR3, VH FR4, VL FR1, VL FR2, VL FR3, and VL FR4 are according to North. In some embodiments, VH FR1, VH FR2, VH FR3, VH FR4, VL FR1, VL FR2, VL FR3, and VL FR4 are according to AHo.

In some embodiments, the VH and VL comprise or consist of the amino acid sequences set forth in SEQ ID NOs. (i) 45 and 37, respectively; (ii) 46 and 37, respectively; (iii) 48 and 37, respectively; (iv) 50 and 37, respectively; (v) 52 and 37, respectively; (vi) 54 and 37, respectively; (vii) 56 and 37, respectively; (viii) 58 and 37, respectively; (ix) 60 and 37, respectively; (x) 63 and 37, respectively; (xi) 65 and 37, respectively; (xii) 45 and 66, respectively; (xiii) 46 and 66, respectively; (xiv) 48 and 66, respectively; (xv) 50 and 66, respectively; (xvi) 52 and 66, respectively; (xvii) 54 and 66, respectively; (xviii) 56 and 66, respectively; (xix) 58 and 66, respectively; (xx) 60 and 66, respectively; (xxi) 63 and 66, respectively; (xxii) 65 and 66, respectively; (xxiii) 45 and 68, respectively; (xxiv) 46 and 68, respectively; (xxv) 48 and 68, respectively; (xxvi) 50 and 68, respectively; (xxvii) 52 and 68, respectively; (xxviii) 54 and 68, respectively; (xxix) 56 and 68, respectively; (xxx) 58 and 68, respectively; (xxxi) 60 and 68, respectively; (xxxii) 63 and 68, respectively; (xxxiii) 65 and 68, respectively; (xxxiv) 45, 46, 48, 50, 52, 54, 56, 58, 60, 63, or 65, and 8, respectively; (xxxv) 45, 46, 48, 50, 52, 54, 56, 58, 60, 63, or 65, and 17, respectively; (xxxvi) 45, 46, 48, 50, 52, 54, 56, 58, 60, 63, or 65, and 20, respectively; (xxxvii) 45, 46, 48, 50, 52, 54, 56, 58, 60, 63, or 65, and 23, respectively; (xxxviii) 45, 46, 48, 50, 52, 54, 56, 58, 60, 63, or 65, and 31, respectively; (xxxix) 2, 45, 46, 48, 50, 52, 54, 56, 58, 60, 63, or 65, and 37, respectively; (xxxx) 2, 45, 46, 48, 50, 52, 54, 56, 58, 60, 63, or 65, and 66, respectively; (xxxxi) 2, 45, 46, 48, 50, 52, 54, 56, 58, 60, 63, or 65, and 68, respectively; (xxxxii) 2, 14, 45, 46, 48, 50, 52, 54, 56, 58, 60, 63, or 65, and 66, respectively; (xxxxiii) 2 and 8, respectively; or (xxxxiv) 2 and 37, respectively.

In certain embodiments, the VH and VL comprise or consist of the amino acid sequences set forth in SEQ ID NOs:54 and 8, respectively.

In certain embodiments, an antibody comprising a heavy chain and a light chain is provided, wherein the heavy chain and the light chain comprise the amino acid sequences set forth in SEQ ID NOs: (i) 45 and 37, respectively; (ii) 46 and 37, respectively; (iii) 48 and 37, respectively; (iv) 50 and 37, respectively; (v) 52 and 37, respectively; (vi) 54 and 37, respectively; (vii) 56 and 37, respectively; (viii) 58 and 37, respectively; (ix) 60 and 37, respectively; (x) 63 and 37, respectively; (xi) 65 and 37, respectively; (xii) 45 and 66, respectively; (xiii) 46 and 66, respectively; (xiv) 48 and 66, respectively; (xv) 50 and 66, respectively; (xvi) 52 and 66, respectively; (xvii) 54 and 66, respectively; (xviii) 56 and 66, respectively; (xix) 58 and 66, respectively; (xx) 60 and 66, respectively; (xxi) 63 and 66, respectively; (xxii) 65 and 66, respectively; (xxiii) 45 and 68, respectively; (xxiv) 46 and 68, respectively; (xxv) 48 and 68, respectively; (xxvi) 50 and 68, respectively; (xxvii) 52 and 68, respectively; (xxviii) 54 and 68, respectively; (xxix) 56 and 68, respectively; (xxx) 58 and 68, respectively; (xxxi) 60 and 68, respectively; (xxxii) 63 and 68, respectively; (xxxiii) 65 and 68, respectively; (xxxiv) 45, 46, 48, 50, 52, 54, 56, 58, 60, 63, or 65, and 8, respectively; (xxxv) 45, 46, 48, 50, 52, 54, 56, 58, 60, 63, or 65, and 17, respectively; (xxxvi) 45, 46, 48, 50, 52, 54, 56, 58, 60, 63, or 65, and 20, respectively; (xxxvii) 45, 46, 48, 50, 52, 54, 56, 58, 60, 63, or 65, and 23, respectively; (xxxviii) 45, 46, 48, 50, 52, 54, 56, 58, 60, 63, or 65, and 31, respectively; (xxxix) 2, 45, 46, 48, 50, 52, 54, 56, 58, 60, 63, or 65, and 37, respectively; (xxxx) 2, 45, 46, 48, 50, 52, 54, 56, 58, 60, 63, or 65, and 66, respectively; (xxxxi) 2, 45, 46, 48, 50, 52, 54, 56, 58, 60, 63, or 65, and 68, respectively; (xxxxii) 2, 14, 45, 46, 48, 50, 52, 54, 56, 58, 60, 63, or 65, and 66, respectively; (xxxxiii) 2 and 8, respectively; or (xxxxiv) 2 and 37, respectively.

In certain embodiments, the heavy chain and the light chain comprise the amino acid sequences set forth in SEQ ID NOs:54 and 8, respectively.

In some embodiments, an anti-influenza neuraminidase (anti-NA) antibody, or an antigen-binding fragment thereof is provided that comprises (i) a heavy chain variable domain (VH) comprising a complementarity determining region (CDR)H1, a CDRH2, and a CDRH3, and (ii) and a light chain variable domain (VL) comprising a CDRL1, a CDRL2, and a CDRL3, wherein: (a) CDRH2 is as set forth in SEQ ID NO.4 and is comprised in SEQ ID NO:59, CDRH1 is as set forth in SEQ ID NO:3, and CDRH3 is as set forth in SEQ ID NO:5; (b) CDRH2 is as set forth in SEQ ID NO:61 and is comprised in SEQ ID NO.: 62, CDRH1 is as set forth in SEQ ID NO:3, and CDRH3 is as set forth in SEQ ID NO:5; or (c) CDRH2 is as set forth in SEQ ID NO:61 and is comprised in SEQ ID NO:64, CDRH1 is as set forth in SEQ ID NO:3, and CDRH3 is as set forth in SEQ ID NO.: 5.

In further embodiments, CDRL1 is as set forth in SEQ ID NO:9, CDRL2 is as set forth in SEQ ID NO:10, and CDRL3 is as set forth in any one of SEQ ID NOs:11, 18, 21, 24, 33, and 67. In other embodiments, CDRL1 is as set forth in SEQ ID NO:32, CDRL2 is as set forth in SEQ ID NO:10, and CDRL3 is as set forth in any one of SEQ ID NOs:11, 18, 21, 24, 33, and 67. In other embodiments, CDRL1 is as set forth in SEQ ID NO:32, CDRL2 is as set forth in SEQ ID NO:96, and CDRL3 is as set forth in SEQ ID NO:33.

In some embodiments, an anti-influenza neuraminidase (anti-NA) antibody, or an antigen-binding fragment thereof is provided that comprises a heavy chain variable domain (VH) and a light chain variable domain (VL), wherein the VH and VL comprise or consist of the amino acid sequences set forth in SEQ ID NOs: (i) 65 and 68, respectively; (ii) 46 and 8, respectively; (iii) 48 and 8, respectively; (iv) 50 and 8, respectively; (v) 52 and 8, respectively; (vi) 54 and 8, respectively; (vii) 56 and 8, respectively; (viii) 58 and 8, respectively; (ix) 60 and 8, respectively; (x) 63 and 8, respectively; (xi) 46 and 66, respectively; (xii) 48 and 66, respectively; (xiii) 50 and 66, respectively; (xiv) 52 and 66, respectively; (xv) 54 and 66, respectively; (xvi) 56 and 66, respectively; (xvii) 58 and 66, respectively; (xviii) 60 and 66, respectively; (xix) 63 and 66, respectively; or (xx) 2 and 37, respectively.

In some embodiments, the influenza comprises an influenza A virus, an influenza B virus, or both.

In some embodiments, the antibody, or the antigen-binding fragment, comprises a human antibody, a monoclonal antibody, a purified antibody, a single chain antibody, a Fab, a Fab′, a F(ab′)2, or Fv.

In some embodiments, the antibody or antigen-binding fragment is a multi-specific antibody or antigen-binding fragment, wherein, optionally, the antibody or antigen-binding fragment is a bi-specific antibody or antigen-binding fragment.

In some embodiments, the antibody or antigen-binding fragment comprises an (e.g., IgG1) Fc polypeptide or a fragment thereof. In some embodiments, the antibody or antigen-binding fragment comprises a IgG, IgA, IgM, IgE, or IgD isotype. In further embodiments, the antibody or antigen-binding fragment comprises an IgG isotype selected from IgG1, IgG2, IgG3, and IgG4. In certain embodiments, the antibody or antigen-binding fragment comprises an IgG1 isotype. In some embodiments, the antibody or antigen-binding fragment comprises an IgG1m3 allotype, an IgG1m17 allotype, an IgG1 ml allotype, or any combination thereof.

In some embodiments, the Fc polypeptide or fragment thereof comprises: (i) a mutation that increases binding affinity to a human FcRn (e.g., as measured using surface plasmon resonance (SPR) (e.g., Biacore, e.g., T200 instrument, using manufacturer's protocols)), as compared to a reference Fc polypeptide that does not comprise the mutation; and/or (ii) a mutation that increases binding affinity to a human FcγR (e.g., as measured using surface plasmon resonance (SPR) (e.g., Biacore, e.g., T200 instrument, using manufacturer's protocols, and/or as measured using mesoscale discovery (MSD))) as compared to a reference Fc polypeptide that does not comprise the mutation.

In some embodiments, the mutation that increases binding affinity to a human FcRn comprises: M428L; N434S; N434H; N434A; N434S; M252Y; S254T; T256E; T250Q; P257I; Q311I; D376V; T307A; E380A; or any combination thereof. In some embodiments, the mutation that increases binding affinity to a human FcRn comprises: (i) M428L/N434S; (ii) M252Y/S254T/T256E; (iii) T250Q/M428L; (iv) P257I/Q311I; (v) P257I/N434H; (vi) D376V/N434H; (vii) T307A/E380A/N434A; (viii) M428L/N434A; or (ix) any combination of (i)-(viii). In some embodiments, the mutation that increases binding affinity to a human FcRn comprises M428L/N434S or M428L/N434A.

In some embodiments, the mutation that enhances binding to a FcγR comprises S239D; I332E; A330L; G236A; or any combination thereof. In some embodiments, the mutation that enhances binding to a FcγR comprises: (i) S239D/I332E; (ii) S239D/A330L/I332E; (iii) G236A/S239D/I332E; or (iv) G236A/A330L/I332E, wherein the Fc polypeptide or fragment thereof optionally comprises Ser at position 239.

In some embodiments, the antibody or antigen-binding fragment comprises a mutation that alters glycosylation, wherein the mutation that alters glycosylation comprises N297A, N297Q, or N297G, and/or which is aglycosylated, and/or which is afucosylated.

In some embodiments, an anti-influenza neuraminidase (anti-NA) antibody, or an antigen-binding fragment thereof, is provided that comprises (i) in a heavy chain, (i)(a) a variable domain (VH) comprising the complementarity determining region (CDR)H1, CDRH2, and CDRH3 amino acid sequences set forth in SEQ ID NOs:3-5, respectively, and (i)(b) the mutations M428L and N434A; and (ii) in a light chain, a light chain variable domain (VL) comprising the CDRL1, CDRL2, and CDRL3 amino acid sequences set forth in SEQ ID NOs:9-11, respectively.

In some embodiments, an anti-influenza neuraminidase (anti-NA) antibody, or an antigen-binding fragment thereof, is provided that comprises (i) in a heavy chain, (i)(a) a variable domain (VH) comprising the complementarity determining region (CDR)H1, CDRH2, and CDRH3 amino acid sequences set forth in SEQ ID NOs:3-5, respectively, and (i)(b) the mutations M428L and N434S; and (ii) in a light chain, a light chain variable domain (VL) comprising the CDRL1, CDRL2, and CDRL3 amino acid sequences set forth in SEQ ID NOs:9-11, respectively.

In certain embodiments, VH and VL have at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity (or similarity) to, or comprise or consist of, the amino acid sequences set forth in SEQ ID NOs:2 and 8, respectively. In certain embodiments, VH and VL comprise or consist of, the amino acid sequences set forth in SEQ ID NOs:2 and 8, respectively.

In certain embodiments, VH and VL have at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity (or similarity) to, or comprise or consist of, the amino acid sequences set forth in SEQ ID NOs:2 and 37, respectively. In certain embodiments, VH and VL comprise or consist of, the amino acid sequences set forth in SEQ ID NOs:2 and 37, respectively.

In certain embodiments, the antibody or antigen-binding fragment comprises an IgG1m3 allotype, an IgG1m17 allotype, an IgG1 ml allotype, or any combination thereof.

In certain embodiments, VH is comprised in a heavy chain that further comprises the CHT-CH3 amino acid sequence set forth in SEQ ID NO:34, SEQ ID NO:36, or SEQ ID NO:38. In certain embodiments, VL is comprised in a light chain that further comprises the CL amino acid sequence set forth in SEQ ID NO:35.

In some embodiments, an anti-influenza neuraminidase (anti-NA) antibody, or an antigen-binding fragment thereof, is provided that comprises (i) a heavy chain comprising or consisting of the amino acid sequence set forth in SEQ ID NO:39, or comprising or consisting of SEQ ID NO:39 with the C-terminal lysine removed; and (ii) a light chain comprising or consisting of the amino acid sequence set forth in SEQ ID NO:41.

In some embodiments, an anti-influenza neuraminidase (anti-NA) antibody, or an antigen-binding fragment thereof, is provided that comprises (i) a heavy chain comprising or consisting of the amino acid sequence set forth in SEQ ID NO:40, or comprising or consisting of SEQ ID NO:40 with the C-terminal lysine removed; and (ii) a light chain comprising or consisting of the amino acid sequence set forth in SEQ ID NO:41. In some embodiments, an anti-influenza neuraminidase (anti-NA) antibody, or an antigen-binding fragment thereof, is provided that comprises (i) a heavy chain comprising or consisting of the amino acid sequence set forth in SEQ ID NO:42, or comprising or consisting of SEQ ID NO:42 with the C-terminal lysine removed; and (ii) a light chain comprising or consisting of the amino acid sequence set forth in SEQ ID NO:44. In some embodiments, an anti-influenza neuraminidase (anti-NA) antibody, or an antigen-binding fragment thereof, is provided that comprises (i) a heavy chain comprising or consisting of the amino acid sequence set forth in SEQ ID NO:43, or comprising or consisting of SEQ ID NO:43 with the C-terminal lysine removed; and (ii) a light chain comprising or consisting of the amino acid sequence set forth in SEQ ID NO:44. In some embodiments, an anti-influenza neuraminidase (anti-NA) antibody, or an antigen-binding fragment thereof, is provided that comprises (i) two heavy chains, each comprising or consisting of the amino acid sequence set forth in SEQ ID NO:39, or comprising or consisting of SEQ ID NO:39 with the C-terminal lysine removed; and (ii) two light chains, each comprising or consisting of the amino acid sequence set forth in SEQ ID NO:41.

In some embodiments, an anti-influenza neuraminidase (anti-NA) antibody, or an antigen-binding fragment thereof, is provided that comprises (i) two heavy chains, each comprising or consisting of the amino acid sequence set forth in SEQ ID NO:40, or comprising or consisting of SEQ ID NO:40 with the C-terminal lysine removed; and (ii) two light chains, each comprising or consisting of the amino acid sequence set forth in SEQ ID NO:41.

In some embodiments, an anti-influenza neuraminidase (anti-NA) antibody, or an antigen-binding fragment thereof, is provided that comprises (i) two heavy chains, each comprising or consisting of the amino acid sequence set forth in SEQ ID NO:42, or comprising or consisting of SEQ ID NO:42 with the C-terminal lysine removed; and (ii) two lights, each chain comprising or consisting of the amino acid sequence set forth in SEQ ID NO:44.

In some embodiments, an anti-influenza neuraminidase (anti-NA) antibody, or an antigen-binding fragment thereof, is provided that comprises (i) two heavy chains, each comprising or consisting of the amino acid sequence set forth in SEQ ID NO:43, or comprising or consisting of SEQ ID NO:43 with the C-terminal lysine removed; and (ii) two light chains, each comprising or consisting of the amino acid sequence set forth in SEQ ID NO:44.

In some embodiments, an anti-influenza neuraminidase (anti-NA) antibody, or an antigen-binding fragment thereof, is provided that comprises, in a heavy chain thereof, the amino acid mutation(s) set forth in any one of (i)-(xviii): (i) G236A, L328V, and Q295E; (ii) G236A, P230A, and Q295E; (iii) G236A, R292P, and 1377N; (iv) G236A, K334A, and Q295E; (v) G236S, R292P, and Y300L; (vi) G236A and Y300L; (vii) G236A, R292P, and Y300L; (viii) G236S, G420V, G446E, and L309T; (ix) G236A and R292P; (x) R292P and Y300L; (xi) G236A and R292P; (xii) Y300L; (xiii) E345K, G236S, L235Y, and S267E; (xiv) E272R, L309T, S219Y, and S267E; (xv) G236Y; (xvi) G236W; (xvii) F243L, G446E, P396L, and S267E; (xviii) G236A, S239D, and H268E.

In certain embodiments, the antibody or antigen-binding fragment comprises, in a heavy chain thereof, the amino acid mutation G236A.

In certain embodiments, the present disclosure provides an antibody comprising: (i) a heavy chain comprising the amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSSASTKGPSVFPLAPSSKSTSGGTAALGCLVKDYFPEPVTVSWNSGALTS GVHTFPAVLQSSGLYSLSSVVTVPSSSLGTQTYICNVNHKPSNTKVDKRVEPKSCDKTHT CPPCPAPELLGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKAKGQ PREPQVYTLPPSREEMTKNQVSLTCLVKGFYPSDIAVEWESNGQPENNYKTTPPVLDSD GSFFLYSKLTVDKSRWQQGNVFSCSVLHEALHSHYTQKSLSLSPGK (SEQ ID NO:107), or SEQ ID NO:107 with the C-terminal lysine removed or the C-terminal glycine-lysine removed; and
(ii) a light chain comprising the amino acid sequence

(SEQ ID NO.: 108)
EIVMTQSPATLSLSSGERATLSCRASRSVSSNLAWYQQKPGQAPRLLIY
DASTRATGFSARFAGSGSGTEFTLTISSLQSEDSAIYYCQQYNNWPPWT
FGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC.

In certain embodiments, the present disclosure provides an antibody comprising: (i) a heavy chain consisting of the amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSSASTKGPSVFPLAPSSKSTSGGTAALGCLVKDYFPEPVTVSWNSGALTS GVHTFPAVLQSSGLYSLSSVVTVPSSSLGTQTYICNVNHKPSNTKVDKRVEPKSCDKTHT CPPCPAPELLGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKAKGQ PREPQVYTLPPSREEMTKNQVSLTCLVKGFYPSDIAVEWESNGQPENNYKTTPPVLDSD GSFFLYSKLTVDKSRWQQGNVFSCSVLHEALHSHYTQKSLSLSPGK (SEQ ID NO.: 107), or SEQ ID NO:107 with the C-terminal lysine removed or the C-terminal glycine-lysine removed; and
(ii) a light chain consisting of the amino acid sequence

(SEQ ID NO.: 108)
EIVMTQSPATLSLSSGERATLSCRASRSVSSNLAWYQQKPGQAPRLLIY
DASTRATGFSARFAGSGSGTEFTLTISSLQSEDSAIYYCQQYNNWPPWT
FGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC.

In certain embodiments, the present disclosure provides an antibody comprising: (i) two heavy chains, wherein each of the two heavy chains comprises the amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSSASTKGPSVFPLAPSSKSTSGGTAALGCLVKDYFPEPVTVSWNSGALTS GVHTFPAVLQSSGLYSLSSVVTVPSSSLGTQTYICNVNHKPSNTKVDKRVEPKSCDKTHT CPPCPAPELLGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKAKGQ PREPQVYTLPPSREEMTKNQVSLTCLVKGFYPSDIAVEWESNGQPENNYKTTPPVLDSD GSFFLYSKLTVDKSRWQQGNVFSCSVLHEALHSHYTQKSLSLSPGK (SEQ ID NO.: 107), or SEQ ID NO:107 with the C-terminal lysine removed or the C-terminal glycine-lysine removed; and
(ii) two light chains, wherein each of the two light chains comprises the amino acid sequence

(SEQ ID NO.: 108)
EIVMTQSPATLSLSSGERATLSCRASRSVSSNLAWYQQKPGQAPRL
LIYDASTRATGFSARFAGSGSGTEFTLTISSLQSEDSAIYYCQQYN
NWPPWTFGQGTKVEIKRTVAAPSVFIFPPSDEQLKSGTASVVCLLN
NFYPREAKVQWKVDNALQSGNSQESVTEQDSKDSTYSLSSTLTLSK
ADYEKHKVYACEVTHQGLSSPVTKSFNRGEC.

In certain embodiments, the present disclosure provides an antibody comprising: (i) two heavy chains, wherein each of the two heavy chains consists of the amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSSASTKGPSVFPLAPSSKSTSGGTAALGCLVKDYFPEPVTVSWNSGALTS GVHTFPAVLQSSGLYSLSSVVTVPSSSLGTQTYICNVNHKPSNTKVDKRVEPKSCDKTHT CPPCPAPELLGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKAKGQ PREPQVYTLPPSREEMTKNQVSLTCLVKGFYPSDIAVEWESNGQPENNYKTTPPVLDSD GSFFLYSKLTVDKSRWQQGNVFSCSVLHEALHSHYTQKSLSLSPGK (SEQ ID NO:107), or SEQ ID NO:107 with the C-terminal lysine removed or the C-terminal glycine-lysine removed; and
(ii) two light chains, wherein each of the two light chains consists of the amino acid sequence

(SEQ ID NO.: 108)
EIVMTQSPATLSLSSGERATLSCRASRSVSSNLAWYQQKPGQAPRL
LIYDASTRATGFSARFAGSGSGTEFTLTISSLQSEDSAIYYCQQYN
NWPPWTFGQGTKVEIKRTVAAPSVFIFPPSDEQLKSGTASVVCLLN
NFYPREAKVQWKVDNALQSGNSQESVTEQDSKDSTYSLSSTLTLSK
ADYEKHKVYACEVTHQGLSSPVTKSFNRGEC.

### Polynucleotides, Vectors, and Host Cells

In another aspect, the present disclosure provides isolated polynucleotides that encode any of the presently disclosed antibodies or an antigen-binding fragment thereof, or a portion thereof (e.g., a CDR, a VH, a VL, a heavy chain, or a light chain, or a heavy chain and a light chain), or that encode a presently disclosed polypeptide.

In certain embodiments, the polynucleotide comprises deoxyribonucleic acid (DNA) or ribonucleic acid (RNA), wherein the RNA optionally comprises messenger RNA (mRNA).

In some embodiments, the polynucleotide comprises a modified nucleoside, a cap-1 structure, a cap-2 structure, or any combination thereof. In certain embodiments, the polynucleotide comprises a pseudouridine, a N6-methyladenonsine, a 5-methylcytidine, a 2-thiouridine, or any combination thereof. In some embodiments, the pseudouridine comprises N1-methylpseudouridine.

In certain embodiments, the polynucleotide is codon-optimized for expression in a host cell (e.g., a human cell, or a CHO cell). Once a coding sequence is known or identified, codon optimization can be performed using known techniques and tools, e.g., using the GenScript® OptimiumGene™ tool, or the like). Codon-optimized sequences include sequences that are partially codon-optimized (i.e., one or more codon is optimized for expression in the host cell) and those that are fully codon-optimized.

It will also be appreciated that polynucleotides encoding antibodies and antigen-binding fragments of the present disclosure may possess different nucleotide sequences while still encoding a same antibody or antigen-binding fragment due to, for example, the degeneracy of the genetic code, splicing, and the like.

In certain embodiments, a polynucleotide comprises (i) a nucleotide sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 98%, or at least 99% identity (or similarity) to the nucleotide sequence set forth in SEQ ID NO:109 and (ii) a nucleotide sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 98%, or at least 99% identity (or similarity) to the nucleotide sequence set forth in SEQ ID NO.: 110. In certain embodiments, the polynucleotide encodes: SEQ ID NO:107 and SEQ ID NO:108.

In certain embodiments, a first polynucleotide comprises a nucleotide sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 98%, or at least 99% identity (or similarity) to the nucleotide sequence set forth in SEQ ID NO:109 and a second polynucleotide comprises a nucleotide sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 98%, or at least 99% identity (or similarity) to the nucleotide sequence set forth in SEQ ID NO:110. In certain embodiments, the first polynucleotide encodes SEQ ID NO:107 and the second polynucleotide encodes SEQ ID NO:108.

In any of the presently disclosed embodiments, the polynucleotide can comprise deoxyribonucleic acid (DNA) or ribonucleic acid (RNA). In some embodiments, the RNA comprises messenger RNA (mRNA).

Vectors are also provided, wherein the vectors comprise or contain a polynucleotide as disclosed herein (e.g., a polynucleotide that encodes an antibody or antigen-binding fragment or polypeptide that binds to IAV NA, IBV NA, or both). A vector can comprise any one or more of the vectors disclosed herein. In particular embodiments, a vector is provided that comprises a DNA plasmid construct encoding the antibody or antigen-binding fragment, or a portion thereof (e.g., so-called “DMAb”; see, e.g., Muthumani et al., J Infect Dis. 214(3):369-378 (2016); Muthumani et al., Hum Vaccin Immunother 9:2253-2262 (2013)); Flingai et al., Sci Rep. 5:12616 (2015); and Elliott et al., NPJ Vaccines 18 (2017), which antibody-coding DNA constructs and related methods of use, including administration of the same, are incorporated herein by reference). In certain embodiments, a DNA plasmid construct comprises a single open reading frame encoding a heavy chain and a light chain (or a VH and a VL) of the antibody or antigen-binding fragment, wherein the sequence encoding the heavy chain and the sequence encoding the light chain are optionally separated by polynucleotide encoding a protease cleavage site and/or by a polynucleotide encoding a self-cleaving peptide. In some embodiments, the substituent components of the antibody or antigen-binding fragment are encoded by a polynucleotide comprised in a single plasmid. In other embodiments, the substituent components of the antibody or antigen-binding fragment are encoded by a polynucleotide comprised in two or more plasmids (e.g., a first plasmid comprises a polynucleotide encoding a heavy chain, VH, or VH+CH1, and a second plasmid comprises a polynucleotide encoding the cognate light chain, VL, or VL+CL). In certain embodiments, a single plasmid comprises a polynucleotide encoding a heavy chain and/or a light chain from two or more antibodies or antigen-binding fragments of the present disclosure. An exemplary expression vector is pVax1, available from Invitrogen®. A DNA plasmid of the present disclosure can be delivered to a subject by, for example, electroporation (e.g., intramuscular electroporation), or with an appropriate formulation (e.g., hyaluronidase).

In some embodiments, a polynucleotide (e.g. mRNA) is provided that comprises (i) a nucleotide sequence encoding SEQ ID NO.: 107, or SEQ ID NO:107 with the C-terminal lysine removed or the C-terminal glycine-lysine removed, and (ii) a nucleotide sequence encoding SEQ ID NO:108. The polynucleotide can be comprised in a composition that further comprises a pharmaceutically acceptable carrier, excipient, or diluent.

In some embodiments, a first plasmid or vector is provided that comprises a polynucleotide (e.g. mRNA) encoding SEQ ID NO:107, or SEQ ID NO:107 with the C-terminal lysine removed or the C-terminal glycine-lysine removed, and a second plasmid or vector is provided that comprises a polynucleotide (e.g. mRNA) encoding SEQ ID NO:108. The first plasmid or vector and/or the second plasmid or vector can be comprised in a composition that further comprises a pharmaceutically acceptable carrier, excipient, or diluent.

In certain embodiments, a first plasmid or vector is provided that comprises a polynucleotide (e.g. mRNA) comprising or consisting of SEQ ID NO:109, and a second plasmid or vector is provided that comprises a polynucleotide (e.g. mRNA) comprising or consisting of SEQ ID NO:110. The first plasmid or vector and/or the second plasmid or vector can be comprised in a composition that further comprises a pharmaceutically acceptable carrier, excipient, or diluent.

In some embodiments, a method is provided that comprises administering to a subject a first polynucleotide (e.g., mRNA) encoding an antibody heavy chain, a VH, or a Fd (VH+CH1), and administering to the subject a second polynucleotide (e.g., mRNA) encoding the cognate antibody light chain, VL, or VL+CL.

In some embodiments, a method is provided that comprises administering to a subject a polynucleotide (e.g. mRNA) that comprises (i) a nucleotide sequence encoding SEQ ID NO:107, or SEQ ID NO.: 107 with the C-terminal lysine removed or the C-terminal glycine-lysine removed, and (ii) a nucleotide sequence encoding SEQ ID NO:108. The polynucleotide can be comprised in a composition that further comprises a pharmaceutically acceptable carrier, excipient, or diluent.

In some embodiments, a method is provided that comprises administering to a subject (i) a first plasmid or vector that comprises a polynucleotide (e.g. mRNA) encoding SEQ ID NO:107, or SEQ ID NO:107 with the C-terminal lysine removed or the C-terminal glycine-lysine removed, and (ii) a second plasmid or vector that comprises a polynucleotide (e.g. mRNA) encoding SEQ ID NO.: 108. The first plasmid or vector and/or the second plasmid or vector can be comprised in a composition that further comprises a pharmaceutically acceptable carrier, excipient, or diluent.

In certain embodiments, a method is provided that comprises administering to a subject a first plasmid or vector that comprises a polynucleotide (e.g. mRNA) comprising or consisting of SEQ ID NO:109, and a second plasmid or vector is provided that comprises a polynucleotide (e.g. mRNA) comprising or consisting of SEQ ID NO:110. The first plasmid or vector and/or the second plasmid or vector can be comprised in a composition that further comprises a pharmaceutically acceptable carrier, excipient, or diluent. In some embodiments, a polynucleotide (e.g., mRNA) is provided that encodes a heavy chain and a light chain of an antibody or antigen-binding fragment thereof. In some embodiments, a polynucleotide (e.g., mRNA) is provided that encodes two heavy chains and two light chains of an antibody or antigen-binding fragment thereof. See, e.g. Li, J Q., Zhang, Z R., Zhang, H Q. et al. Intranasal delivery of replicating mRNA encoding neutralizing antibody against SARS-CoV-2 infection in mice. Sig Transduct Target Ther 6, 369 (2021). https://doi.org/10.1038/s41392-021-00783-1, the antibody-encoding mRNA constructs, vectors, and related techniques of which are incorporated herein by reference. In some embodiments, a polynucleotide is delivered to a subject via an alphavirus replicon particle (VRP) delivery system. In some embodiments, a replicon comprises a modified VEEV replicon comprising two subgenomic promoters. In some embodiments, a polynucleotide or replicon can translate simultaneously the heavy chain (or VH, or VH+1) and the light chain (or VL, or VL+CL) of an antibody or antigen-binding fragment thereof. In some embodiments, a method is provided that comprises delivering to a subject such a polynucleotide or replicon.

In a further aspect, the present disclosure also provides a host cell expressing an antibody or antigen-binding fragment according to the present disclosure; or comprising or containing a vector or polynucleotide according the present disclosure.

Examples of such cells include but are not limited to, eukaryotic cells, e.g., yeast cells, animal cells, insect cells, plant cells; and prokaryotic cells, including E. coli. In some embodiments, the cells are mammalian cells, such as human B cells. In certain such embodiments, the cells are a mammalian cell line such as CHO cells (e.g., DHFR-CHO cells (Urlaub et al., PNAS 77:4216 (1980)), human embryonic kidney cells (e.g., HEK293T cells), PER.C6 cells, Y0 cells, Sp2/0 cells. NS0 cells, human liver cells, e.g. Hepa RG cells, myeloma cells or hybridoma cells. Other examples of mammalian host cell lines include mouse sertoli cells (e.g., TM4 cells); monkey kidney CV1 line transformed by SV40 (COS-7); baby hamster kidney cells (BHK); African green monkey kidney cells (VERO-76); monkey kidney cells (CV1); human cervical carcinoma cells (HELA); human lung cells (W138); human liver cells (Hep G2); canine kidney cells (MDCK; buffalo rat liver cells (BRL 3A); mouse mammary tumor (MMT 060562); TRI cells; MRC 5 cells; and FS4 cells. Mammalian host cell lines suitable for antibody production also include those described in, for example, Yazaki and Wu, Methods in Molecular Biology, Vol. 248 (B. K. C. Lo, ed., Humana Press, Totowa, N.J.), pp. 255-268 (2003).

In certain embodiments, a host cell is a prokaryotic cell, such as an E. coli. The expression of peptides in prokaryotic cells such as E. coli is well established (see, e.g., Pluckthun, A. Bio Technology 9:545-551 (1991). For example, antibodies may be produced in bacteria, in particular when glycosylation and Fc effector function are not needed. For expression of antibody fragments and polypeptides in bacteria, see, e.g., U.S. Pat. Nos. 5,648,237; 5,789,199; and 5,840,523.

In particular embodiments, the cell may be transfected with a vector according to the present description with an expression vector. The term “transfection” refers to the introduction of nucleic acid molecules, such as DNA or RNA (e.g. mRNA) molecules, into cells, such as into eukaryotic cells. In the context of the present description, the term “transfection” encompasses any method known to the skilled person for introducing nucleic acid molecules into cells, such as into eukaryotic cells, including into mammalian cells. Such methods encompass, for example, electroporation, lipofection, e.g., based on cationic lipids and/or liposomes, calcium phosphate precipitation, nanoparticle based transfection, virus based transfection, or transfection based on cationic polymers, such as DEAE-dextran or polyethylenimine, etc. In certain embodiments, the introduction is non-viral.

Moreover, host cells of the present disclosure may be transfected stably or transiently with a vector according to the present disclosure, e.g. for expressing an antibody, or an antigen-binding fragment thereof, according to the present disclosure. In such embodiments, the cells may be stably transfected with the vector as described herein. Alternatively, cells may be transiently transfected with a vector according to the present disclosure encoding an antibody or antigen-binding fragment as disclosed herein. In any of the presently disclosed embodiments, a polynucleotide may be heterologous to the host cell.

Accordingly, the present disclosure also provides recombinant host cells that heterologously express an antibody or antigen-binding fragment of the present disclosure. For example, the cell may be of a species that is different to the species from which the antibody was fully or partially obtained (e.g., CHO cells expressing a human antibody or an engineered human antibody). In some embodiments, the cell type of the host cell does not express the antibody or antigen-binding fragment in nature. Moreover, the host cell may impart a post-translational modification (PTM; e.g., glysocylation or fucosylation), or a lack thereof, on the antibody or antigen-binding fragment that is not present in a native state of the antibody or antigen-binding fragment (or in a native state of a parent antibody from which the antibody or antigen binding fragment was engineered or derived). Such a PTM, or a lack thereof, may result in a functional difference (e.g., reduced immunogenicity). Accordingly, an antibody or antigen-binding fragment of the present disclosure that is produced by a host cell as disclosed herein may include one or more post-translational modification that is distinct from the antibody (or parent antibody) in its native state (e.g., a human antibody produced by a host cell can comprise one or more post-translational modification, or can include fewer post-translational modification(s), such that it is distinct from the antibody when isolated from the human and/or produced by the native human B cell or plasma cell).

Insect cells useful expressing a binding protein of the present disclosure are known in the art and include, for example, Spodoptera frugipera Sf9 cells, Trichoplusia ni BTI-TN5B1-4 cells, and Spodoptera frugipera SfSWT01 “Mimic™” cells. See, e.g., Palmberger et al., J Biotechnol. 153(3-4):160-166 (2011). Numerous baculoviral strains have been identified which may be used in conjunction with insect cells, particularly for transfection of Spodoptera frugiperda cells.

Eukaryotic microbes such as filamentous fungi or yeast are also suitable hosts for cloning or expressing protein-encoding vectors, and include fungi and yeast strains with “humanized” glycosylation pathways, resulting in the production of an antibody with a partially or fully human glycosylation pattern. See Gerngross, Nat. Biotech. 22:1409-1414 (2004); Li et al., Nat. Biotech. 24:210-215 (2006).

Plant cells can also be utilized as hosts for expressing an antibody or antigen-binding fragment of the present disclosure. For example, PLANTIBODIES™ technology (described in, for example, U.S. Pat. Nos. 5,959,177; 6,040,498; 6,420,548; 7,125,978; and 6,417,429) employs transgenic plants to produce antibodies.

In certain embodiments, the host cell comprises a mammalian cell. In particular embodiments, the host cell is a CHO cell, a HEK293 cell, a PER.C6 cell, a Y0 cell, a Sp2/0 cell, a NS0 cell, a human liver cell, a myeloma cell, or a hybridoma cell.

In a related aspect, the present disclosure provides methods for producing an antibody, or antigen-binding fragment, wherein the methods comprise culturing a host cell of the present disclosure under conditions and for a time sufficient to produce the antibody, or the antigen-binding fragment. Methods useful for isolating and purifying recombinantly produced antibodies, by way of example, may include obtaining supernatants from suitable host cell/vector systems that secrete the recombinant antibody into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of the isolated/recombinant antibody described herein include batch cell culture, which is monitored and controlled to maintain appropriate culture conditions. Purification of soluble antibodies may be performed according to methods described herein and known in the art and that comport with laws and guidelines of domestic and foreign regulatory agencies.

### Compositions

Also provided herein are compositions that comprise a presently disclosed antibody, antigen-binding fragment, polypeptide, polynucleotide, vector, or host cell, singly or in any combination, and can further comprise a pharmaceutically acceptable carrier, excipient, or diluent. Such compositions, as well as carriers, excipients, and diluents, are discussed in further detail herein.

In certain embodiments, a composition comprises a first vector comprising a first plasmid, and a second vector comprising a second plasmid, wherein the first plasmid comprises a polynucleotide encoding a heavy chain, VH, or VH+CH1, and a second plasmid comprises a polynucleotide encoding the cognate light chain, VL, or VL+CL of the antibody or antigen-binding fragment thereof. In certain embodiments, a composition comprises a polynucleotide (e.g., mRNA) coupled to a suitable delivery vehicle or carrier. In certain embodiments, a composition comprises a first polynucleotide (e.g., mRNA) encoding an antibody heavy chain, a VH, or a Fd (VH+CH1), and a second polynucleotide (e.g., mRNA) encoding the cognate antibody light chain or VL. In one example, a composition can comprise a first polynucleotide (e.g., mRNA) encoding an antibody heavy chain comprising the VH set forth in SEQ ID NO.: 54 and a second polynucleotide (e.g., mRNA) encoding an antibody light chain comprising the VL set forth in SEQ ID NO:8. In another example, a composition can comprise a first polynucleotide (e.g., mRNA) encoding an antibody heavy chain comprising CDRH1, CDRH2, and CDRH3 sequences as set forth in SEQ ID NOs: (i) 55, 4, and 5, respectively, and a second polynucleotide (e.g., mRNA) encoding an antibody light chain comprising CDRL1, CDRL2, and CDRL3 sequences as set forth in SEQ ID Nos.: 9-11, respectively.

Exemplary vehicles or carriers for administration to a human subject include a lipid or lipid-derived delivery vehicle, such as a liposome, solid lipid nanoparticle, oily suspension, submicron lipid emulsion, lipid microbubble, inverse lipid micelle, cochlear liposome, lipid microtubule, lipid microcylinder, or lipid nanoparticle (LNP) or a nanoscale platform (see, e.g., Li et al. Wilery Interdiscip Rev. Nanomed Nanobiotechnol. 11(2):e1530 (2019)). Principles, reagents, and techniques for designing appropriate mRNA and formulating mRNA-LNP and delivering the same are described in, for example, Pardi et al. (J Control Release 217345-351 (2015)); Thess et al. (Mol Ther 23: 1456-1464 (2015)); Thran et al. (EMBO Mol Med 9(10):1434-1448 (2017); Kose et al. (Sci. Immunol. 4 eaaw6647 (2019); and Sabnis et al. (Mol. Ther. 26:1509-1519 (2018)), which techniques, include capping, codon optimization, nucleoside modification, purification of mRNA, incorporation of the mRNA into stable lipid nanoparticles (e.g., ionizable cationic lipid/phosphatidylcholine/cholesterol/PEG-lipid; ionizable lipid:distearoyl PC:cholesterol:polyethylene glycol lipid), and subcutaneous, intramuscular, intradermal, intravenous, intraperitoneal, and intratracheal administration of the same, are incorporated herein by reference.

In some embodiments, a polynucleotide (e.g. mRNA) is provided that comprises (i) a nucleotide sequence encoding SEQ ID NO:107, or SEQ ID NO:107 with the C-terminal lysine removed or the C-terminal glycine-lysine removed, and (ii) a nucleotide sequence encoding SEQ ID NO:108. The polynucleotide can be comprised in a composition that further comprises a pharmaceutically acceptable carrier, excipient, or diluent.

In some embodiments, a first plasmid or vector is provided that comprises a polynucleotide (e.g. mRNA) encoding SEQ ID NO:107, or SEQ ID NO:107 with the C-terminal lysine removed or the C-terminal glycine-lysine removed, and a second plasmid or vector is provided that comprises a polynucleotide (e.g. mRNA) encoding SEQ ID NO:108. The first plasmid or vector and/or the second plasmid or vector can be comprised in a composition that further comprises a pharmaceutically acceptable carrier, excipient, or diluent.

In certain embodiments, a first plasmid or vector is provided that comprises a polynucleotide (e.g. mRNA) comprising or consisting of SEQ ID NO:109, and a second plasmid or vector is provided that comprises a polynucleotide (e.g. mRNA) comprising or consisting of SEQ ID NO:110. The first plasmid or vector and/or the second plasmid or vector can be comprised in a composition that further comprises a pharmaceutically acceptable carrier, excipient, or diluent.

In certain embodiments, a composition comprises a first antibody or antigen-binding fragment of the present disclosure and a second antibody or antigen-binding fragment of the present disclosure, wherein of the first antibody or antigen-binding fragment and the second antibody or antigen-binding fragment are different.

### METHODS AND USES

Also provided herein are methods for use of an antibody or antigen-binding fragment, nucleic acid, vector, cell, or composition of the present disclosure in the diagnosis of an influenza infection (e.g., in a human subject, or in a sample obtained from a human subject).

Methods of diagnosis (e.g., in vitro, ex vivo) may include contacting an antibody, antibody fragment (e.g., antigen binding fragment) with a sample. Such samples may be isolated from a subject, for example an isolated tissue sample taken from, for example, nasal passages, sinus cavities, salivary glands, lung, liver, pancreas, kidney, ear, eye, placenta, alimentary tract, heart, ovaries, pituitary, adrenals, thyroid, brain, skin or blood. The methods of diagnosis may also include the detection of an antigen/antibody complex, in particular following the contacting of an antibody or antibody fragment with a sample. Such a detection step can be performed at the bench, i.e. without any contact to the human or animal body. Examples of detection methods are well-known to the person skilled in the art and include, e.g., ELISA (enzyme-linked immunosorbent assay), including direct, indirect, and sandwich ELISA.

Also provided herein are methods of treating a subject using an antibody or antigen-binding fragment of the present disclosure, or a composition comprising the same, wherein the subject has, is believed to have, or is at risk for having an infection by influenza. “Treat,” “treatment,” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising an antibody or composition of the present disclosure is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay or prevention of disease progression; remission; survival; prolonged survival; or any combination thereof. In certain embodiments, therapeutic or prophylactic/preventive benefit includes reduction or prevention of hospitalization for treatment of an influenza infection (i.e., in a statistically significant manner). In certain embodiments, therapeutic or prophylactic/preventive benefit includes a reduced duration of hospitalization for treatment of an influenza infection (i.e., in a statistically significant manner). In certain embodiments, therapeutic or prophylactic/preventive benefit includes a reduced or abrogated need for respiratory intervention, such as intubation and/or the use of a respirator device. In certain embodiments, therapeutic or prophylactic/preventive benefit includes reversing a late-stage disease pathology and/or reducing mortality.

In certain embodiments, for a single dose, e.g. a daily, weekly or monthly dose, the amount of the antibody or antigen-binding fragment in a composition or method according to the present disclosure, may not exceed 1 g or 500 mg. In some embodiments, for a single dose, the amount of the antibody or antigen-binding fragment in a composition or method according to the present disclosure, may not exceed 200 mg, or 100 mg. For example, in some embodiments, for a single dose, the amount of the antibody or antigen-binding fragment in a composition or method according to the present disclosure, may not exceed 50 mg.

A “therapeutically effective amount” or “effective amount” of an antibody, antigen-binding fragment, polynucleotide, vector, host cell, or composition of this disclosure refers to an amount of the composition or molecule sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially, sequentially, or simultaneously.

Accordingly, in certain embodiments, methods are provided for treating an influenza infection in a subject, wherein the methods comprise administering to the subject an effective amount of an antibody, antigen-binding fragment, polynucleotide, vector, host cell, or composition as disclosed herein.

Subjects that can be treated by the present disclosure are, in general, human and other primate subjects, such as monkeys and apes for veterinary medicine purposes. Other model organisms, such as mice and rats, may also be treated according to the present disclosure. In any of the aforementioned embodiments, the subject may be a human subject. The subjects can be male or female and can be any suitable age, including infant, juvenile, adolescent, adult, and geriatric subjects.

A number of criteria are believed to contribute to high risk for severe symptoms or death associated with an influenza infection. These include, but are not limited to, age, occupation, general health, pre-existing health conditions, locale, and lifestyle habits. In some embodiments, a subject treated according to the present disclosure comprises one or more risk factors.

In certain embodiments, a human subject treated according to the present disclosure is an infant, a child, a young adult, an adult of middle age, or an elderly person. In certain embodiments, a human subject treated according to the present disclosure is less than 1 year old, or is 1 to 5 years old, or is between 5 and 125 years old (e.g., 5, 10, 15, 20, 25, 30, 35, 40, 45, 50, 55, 60, 65, 70, 75, 80, 85, 90, 95, 100, 105, 110, 115, or 125 years old, including any and all ages therein or therebetween). In certain embodiments, a human subject treated according to the present disclosure is 0-19 years old, 20-44 years old, 45-54 years old, 55-64 years old, 65-74 years old, 75-84 years old, or 85 years old, or older. Persons of middle, and especially of elderly age can be at particular risk. In particular embodiments, the human subject is 45-54 years old, 55-64 years old, 65-74 years old, 75-84 years old, or 85 years old, or older. In some embodiments, the human subject is male. In some embodiments, the human subject is female.

In certain embodiments, a subject treated according to the present disclosure has received a vaccine for influenza and the vaccine is determined to be ineffective, e.g., by post-vaccine infection or symptoms in the subject, by clinical diagnosis or scientific or regulatory consensus.

Prophylaxis of infection with influenza virus refers in particular to prophylactic settings, wherein the subject was not diagnosed with infection with influenza virus (either no diagnosis was performed or diagnosis results were negative) and/or the subject does not show or experience symptoms of infection with influenza virus. Prophylaxis of infection with influenza virus is particularly useful in subjects at greater risk of severe disease or complications when infected, such as pregnant women, children (such as children under 59 months), the elderly, individuals with chronic medical conditions (such as chronic cardiac, pulmonary, renal, metabolic, neurodevelopmental, liver or hematologic diseases) and individuals with immunosuppressive conditions (such as HIV/AIDS, receiving chemotherapy or steroids, or malignancy). Moreover, prophylaxis of infection with influenza virus is also particularly useful in subjects at greater risk acquiring influenza virus infection, e.g., due to increased exposure, for example subjects working or staying in public areas, in particular health care workers.

In certain embodiments, treatment is administered as peri-exposure or pre-exposure prophylaxis. In certain embodiments, treatment is administered as post-exposure prophylaxis.

In some embodiments, a method is provided for reducing the risk of an influenza infection in a subject, wherein the method comprises administering to the subject an effective amount of a presently disclosed antibody, antigen-binding fragment, polynucleotide, vector, host cell, human B cell, or composition. In some embodiments, the antibody or antigen-binding fragment, or the encoded antibody or antigen-binding fragment, comprises a heavy chain comprising SEQ ID NO:54 (and optionally, M428L and N434S mutations in the Fc) and a light chain comprising SEQ ID NO:8. In further embodiments, the heavy chain comprises SEQ ID NO:107 or SEQ ID NO:107 with the C-terminal lysine or the C-terminal glycine-lysine removed, and the light chain comprises SEQ ID NO:108.

In some embodiments, a method is provided for pre-exposure prophylaxis against an influenza infection in a subject, wherein the method comprises administering to the subject an effective amount of a presently disclosed antibody, antigen-binding fragment, polynucleotide, vector, host cell, human B cell, or composition. In some embodiments, the antibody or antigen-binding fragment, or the encoded antibody or antigen-binding fragment, comprises a heavy chain comprising SEQ ID NO:54 (and optionally, M428L and N434S mutations in the Fc) and a light chain comprising SEQ ID NO:8. In further embodiments, the heavy chain comprises SEQ ID NO:107 or SEQ ID NO:107 with the C-terminal lysine or the C-terminal glycine-lysine removed, and the light chain comprises SEQ ID NO:108.

In some embodiments, a method is provided for peri-exposure prophylaxis against an influenza infection in a subject, wherein the method comprises administering to the subject an effective amount of a presently disclosed antibody, antigen-binding fragment, polynucleotide, vector, host cell, human B cell, or composition. In some embodiments, the antibody or antigen-binding fragment, or the encoded antibody or antigen-binding fragment, comprises a heavy chain comprising SEQ ID NO:54 (and optionally, M428L and N434S mutations in the Fc) and a light chain comprising SEQ ID NO:8. In further embodiments, the heavy chain comprises SEQ ID NO:107 or SEQ ID NO:107 with the C-terminal lysine or the C-terminal glycine-lysine removed, and the light chain comprises SEQ ID NO:108.

In some embodiments, a method is provided for peri-exposure prophylaxis against an influenza infection in a subject, wherein the method comprises administering to the subject an effective amount of a presently disclosed antibody, antigen-binding fragment, polynucleotide, vector, host cell, human B cell, or composition. In some embodiments, the antibody or antigen-binding fragment, or the encoded antibody or antigen-binding fragment, comprises a heavy chain comprising SEQ ID NO.: 54 (and optionally, M428L and N434S mutations in the Fc) and a light chain comprising SEQ ID NO:8. In further embodiments, the heavy chain comprises SEQ ID NO:107 or SEQ ID NO.: 107 with the C-terminal lysine or the C-terminal glycine-lysine removed, and the light chain comprises SEQ ID NO:108.

In therapeutic settings, in contrast, the subject is typically infected with influenza virus, diagnosed with influenza virus infection, and/or showing symptoms of influenza virus infection. Of note, the terms “treatment” and “therapy”/“therapeutic” of influenza virus infection can refer to (complete) cure as well as attenuation/reduction of influenza virus infection and/or related symptoms (e.g., attenuation/reduction of severity of infection and/or symptoms, number of symptoms, duration of infection and/or symptoms, or any combination thereof).

It will be understood that reference herein to a reduced number and/or severity of symptoms, which reduction results from administration of a presently disclosed pharmaceutical composition, describes a comparison with a reference subject who did not receive a disclosed pharmaceutical composition. A reference subject can be, for example, (i) the same subject during an earlier period of time (e.g., a prior influenza A virus season), (ii) a subject of a same or a similar: age or age group; gender; pregnancy status; chronic medical condition (such as chronic cardiac, pulmonary, renal, metabolic, neurodevelopmental, liver or hematologic diseases) or lack thereof; and/or immunosuppressive condition or lack thereof; or (iii) a typical subject within a population (e.g., local, regional, or national, including of a same or similar age or age range and/or general state of health) during an influenza virus season. Prophylaxis can be determined by, for example, the failure to develop a diagnosed influenza infection and/or the lack of symptoms associated with influenza infection during a part of a full influenza season, or over a full influenza season.

In certain embodiments, the methods provided herein include administering a therapeutically effective amount of a composition according to the present disclosure to a subject at immediate risk of influenza infection. An immediate risk of influenza infection typically occurs during an influenza epidemic. Influenza viruses are known to circulate and cause seasonal epidemics of disease (WHO, Influenza (Seasonal) Fact sheet, Nov. 6, 2018). In temperate climates, seasonal epidemics occur mainly during winter, while in tropical regions, influenza may occur throughout the year, causing outbreaks more irregularly. For example, in the northern hemisphere, the risk of an influenza epidemic is high during November, December, January, February and March, while in the southern hemisphere the risk of an influenza epidemic is high during May, June, July, August and September.

In some embodiments, treatment and/or prevention comprises post-exposure prophylaxis.

In some embodiments, the subject has received, is receiving, or will receive an antiviral agent. In some embodiments, the antiviral agent comprises a neuraminidase inhibitor, an influenza polymerase inhibitor, or both. In certain embodiments, the antiviral agent comprises oseltamivir, lanamivir, peramivir, zanamivir, baloxavir, or any combination thereof.

Typical routes of administering the presently disclosed compositions include, without limitation, oral, topical, transdermal, inhalation, parenteral, sublingual, buccal, rectal, vaginal, and intranasal. The term “parenteral”, as used herein, includes subcutaneous injections, intravenous, intramuscular, intrasternal injection or infusion techniques. In certain embodiments, administering comprises administering by a route that is selected from oral, intravenous, parenteral, intragastric, intrapleural, intrapulmonary, intrarectal, intradermal, intraperitoneal, intratumoral, subcutaneous, topical, transdermal, intracisternal, intrathecal, intranasal, and intramuscular. In particular embodiments, a method comprises orally administering the antibody, antigen-binding fragment, polynucleotide, vector, host cell, or composition to the subject.

Pharmaceutical compositions according to certain embodiments of the present invention are formulated so as to allow the active ingredients contained therein to be bioavailable upon administration of the composition to a patient. Compositions that will be administered to a subject or patient may take the form of one or more dosage units, where for example, a tablet may be a single dosage unit, and a container of a herein described an antibody or antigen-binding in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain an effective amount of an antibody or antigen-binding fragment, polynucleotide, vector, host cell, or composition of the present disclosure, for treatment of a disease or condition of interest in accordance with teachings herein.

A composition may be in the form of a solid or liquid. In some embodiments, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi solid, semi liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the composition is in the form of a capsule, for example, a gelatin capsule, it may contain, in addition to materials of the above type, a liquid carrier such as polyethylene glycol or oil.

The composition may be in the form of a liquid, for example, an elixir, syrup, solution, emulsion or suspension. The liquid may be for oral administration or for delivery by injection, as two examples. When intended for oral administration, preferred compositions contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

Liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid composition intended for either parenteral or oral administration should contain an amount of an antibody or antigen-binding fragment as herein disclosed such that a suitable dosage will be obtained. Typically, this amount is at least 0.01% of the antibody or antigen-binding fragment in the composition. When intended for oral administration, this amount may be varied to be between 0.1 and about 70% of the weight of the composition. Certain oral pharmaceutical compositions contain between about 4% and about 75% of the antibody or antigen-binding fragment. In certain embodiments, pharmaceutical compositions and preparations according to the present invention are prepared so that a parenteral dosage unit contains between 0.01 to 10% by weight of antibody or antigen-binding fragment prior to dilution.

The composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

A composition may include various materials which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The composition in solid or liquid form may include an agent that binds to the antibody or antigen-binding fragment of the disclosure and thereby assists in the delivery of the compound. Suitable agents that may act in this capacity include monoclonal or polyclonal antibodies, one or more proteins or a liposome. The composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi phasic, or tri phasic systems in order to deliver the active ingredient(s). Delivery of the aerosol includes the necessary container, activators, valves, subcontainers, and the like, which together may form a kit. One of ordinary skill in the art, without undue experimentation, may determine preferred aerosols.

It will be understood that compositions of the present disclosure also encompass carrier molecules for polynucleotides, as described herein (e.g., lipid nanoparticles, nanoscale delivery platforms, and the like).

The pharmaceutical compositions may be prepared by methodology well known in the pharmaceutical art. For example, a composition intended to be administered by injection can be prepared by combining a composition that comprises an antibody, antigen-binding fragment thereof, or antibody conjugate as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the peptide composition so as to facilitate dissolution or homogeneous suspension of the antibody or antigen-binding fragment thereof in the aqueous delivery system.

In any of the presently disclosed embodiments, an antibody or antigen-binding fragment of a composition can comprise CDRH1, CDRH2, CDRH3, CDRL1, CDRL2, and CDRL3 amino acid sequences as set forth in SEQ ID NOs:55, 4, 5, 9, 10, and 11, respectively. In certain embodiments, an antibody or antigen-binding fragment of a composition comprises the VH and VL amino acid sequences set forth in SEQ ID NOs:54 and 8, respectively. In certain embodiments, an antibody or antigen-binding fragment of a composition comprises a human IgG1 isotype (e.g., comprising an allotype such as IgG1m3 or IgG1m17,1) and comprises M428L and N434S mutations in the Fc. In some embodiments, an antibody or antigen-binding fragment of a composition comprises the heavy chain amino acid sequence set forth in SEQ ID NO:107 (or SEQ ID NO:107 with the C-terminal lysine removed or SEQ ID NO:107 with the C-terminal glycine-lysine removed) and the light chain amino acid sequence set forth in SEQ ID NO:108. In some embodiments, an antibody or antigen-binding fragment of a composition comprises two heavy chains each comprising or consisting of the amino acid sequence set forth in SEQ ID NO:107 (or SEQ ID NO:107 with the C-terminal lysine removed or SEQ ID NO:107 with the C-terminal glycine-lysine removed) and two light chains each comprising or consisting of the amino acid sequence set forth in SEQ ID NO.: 108. In some embodiments, a composition is provided that comprises the antibody or antigen-binding fragment at a concentration of 3 mg/kg, 0.9 mg/kg, or 0.3 mg/kg relative to the weight (kg) of a subject in need of the composition. In certain embodiments, the composition comprising the antibody or antigen-binding fragment: has an osmolality of 280-315 mOsm/kg; has no detectable endotoxin with a sensitivity <0.05 EU/mg; is sterile; has a pH of 7.4; and comprises the antibody or antigen-binding fragment purified by affinity chromatography.

In general, an appropriate dose and treatment regimen provide the composition(s) in an amount sufficient to provide therapeutic and/or prophylactic benefit (such as described herein, including an improved clinical outcome (e.g., a decrease in frequency, duration, or severity of diarrhea or associated dehydration, or inflammation, or longer disease-free and/or overall survival, or a lessening of symptom severity). For prophylactic use, a dose should be sufficient to prevent, delay the onset of, or diminish the severity of a disease associated with disease or disorder. Prophylactic benefit of the compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Compositions are administered in an effective amount (e.g., to treat an influenza virus infection), which will vary depending upon a variety of factors including the activity of the specific compound employed; the metabolic stability and length of action of the compound; the age, body weight, general health, sex, and diet of the subject; the mode and time of administration; the rate of excretion; the drug combination; the severity of the particular disorder or condition; and the subject undergoing therapy. In certain embodiments, following administration of therapies according to the formulations and methods of this disclosure, test subjects will exhibit about a 10% up to about a 99% reduction in one or more symptoms associated with the disease or disorder being treated as compared to placebo-treated or other suitable control subjects.

Generally, a therapeutically effective dose of an antibody or antigen binding fragment is (for a 70 kg mammal) from about 0.001 mg/kg (i.e., 0.07 mg) to about 100 mg/kg (i.e., 7.0 g); preferably a therapeutically effective dose is (for a 70 kg mammal) from about 0.01 mg/kg (i.e., 0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., 1.75 g). For polynucleotides, vectors, host cells, and related compositions of the present disclosure, a therapeutically effective dose may be different than for an antibody or antigen-binding fragment.

In some embodiments, a method or use comprises administering a dose of the antibody or antigen-binding fragment to a subject in need thereof, wherein the dose comprises, consists essentially of, or consists of 3 mg/kg, 0.9 mg/kg, or 0.3 mg/kg of the antibody or antigen-binding fragment thereof. In some embodiments, a method or use comprises administering a dose of a composition comprising the antibody or antigen-binding fragment to a subject in need thereof, wherein the dose comprises, consists essentially of, or consists of 3 mg/kg, 0.9 mg/kg, or 0.3 mg/kg of the composition. In some embodiments, a composition is provided that comprises the antibody or antigen-binding fragment at a concentration of 3 mg/kg, 0.9 mg/kg, or 0.3 mg/kg relative to the weight (kg) of a subject in need of the composition. In any of the presently disclosed embodiments, the antibody or antigen-binding fragment can comprise CDRH1, CDRH2, CDRH3, CDRL1, CDRL2, and CDRL3 amino acid sequences as set forth in SEQ ID NOs:55, 4, 5, 9, 10, and 11, respectively. In certain embodiments, the antibody or antigen-binding fragment comprises the VH and VL amino acid sequences set forth in SEQ ID NOs:54 and 8, respectively. In certain embodiments, the antibody or antigen-binding fragment comprises a human IgG1 isotype (e.g., comprising an allotype such as IgG1m3 or IgG1m17,1) and comprises M428L and N434S mutations in the Fc. In some embodiments, the antibody or antigen-binding fragment comprises the heavy chain amino acid sequence set forth in SEQ ID NO:107 (or SEQ ID NO:107 with the C-terminal lysine removed or SEQ ID NO:107 with the C-terminal glycine-lysine removed) and the light chain amino acid sequence set forth in SEQ ID NO:108. In some embodiments, the antibody or antigen-binding fragment comprises two heavy chains each comprising or consisting of the amino acid sequence set forth in SEQ ID NO:107 (or SEQ ID NO:107 with the C-terminal lysine removed or SEQ ID NO:107 with the C-terminal glycine-lysine removed) and two light chains each comprising or consisting of the amino acid sequence set forth in SEQ ID NO:108. In some embodiments, administration comprises intravenous administration to the subject. In some embodiments, a composition is formulated for intravenous administration to the subject. In some embodiments, the method or use comprises a single administration of the antibody, antigen-binding fragment, or composition. In some embodiments, the method or use comprises a single dose of the antibody, antigen-binding fragment, or composition. In certain embodiments, the composition comprising the antibody or antigen-binding fragment: has an osmolality of 280-315 mOsm/kg; has no detectable endotoxin with a sensitivity <0.05 EU/mg; is sterile; has a pH of 7.4; and comprises the antibody or antigen-binding fragment purified by affinity chromatography. In some embodiments, the subject has a weight of about 70 kg. In any of the presently disclosed embodiments, the subject has an H5N1 influenza infection, is at risk of contracting an H5N1 influenza infection, or has been exposed to an H5N1 influenza. In any of the presently disclosed embodiments, the subject has an H7N9 influenza infection, is at risk of contracting an H7N9 influenza infection, or has been exposed to an H7N9 influenza. In some embodiments, a method, use, dose, or composition is for prophylaxis against an influenza infection, such as infection by an influenza H5N1 and/or by an influenza H7N9. In some embodiments, prophylaxis comprises post-exposure prophylaxis.

In certain embodiments, a method comprises administering the antibody, antigen-binding fragment, polynucleotide, vector, host cell, or composition to the subject at 2, 3, 4, 5, 6, 7, 8, 9, 10 times, or more. In certain embodiments, a method comprises administering the antibody, antigen-binding fragment, polynucleotide, vector, host cell, or composition to the subject at least 2, 3, 4, 5, 6, 7, 8, 9, 10 times, or more.

In certain embodiments, a method comprises administering the antibody, antigen-binding fragment, or composition to the subject a plurality of times, wherein a second or successive administration is performed at about 6, about 7, about 8, about 9, about 10, about 11, about 12, about 24, about 48, about 74, about 96 hours, or more, following a first or prior administration, respectively.

In certain embodiments, a method comprises administering the antibody, antigen-binding fragment, polynucleotide, vector, host cell, or composition at least one time prior to the subject being infected by influenza.

Compositions comprising an antibody, antigen-binding fragment, polynucleotide, vector, host cell, or composition of the present disclosure may also be administered simultaneously with, prior to, or after administration of one or more other therapeutic agents, such as, for example, a neuraminidase inhibitor, e.g., oseltamivir, zanamivir, peramivir, or laninamivir. Such combination therapy may include administration of a single pharmaceutical dosage formulation which contains a compound of the invention and one or more additional active agents, as well as administration of compositions comprising an antibody or antigen-binding fragment of the disclosure and each active agent in its own separate dosage formulation. For example, an antibody or antigen-binding fragment thereof as described herein and the other active agent can be administered to the patient together in a single oral dosage composition such as a tablet or capsule, or each agent administered in separate oral dosage formulations. Similarly, an antibody or antigen-binding fragment as described herein and the other active agent can be administered to the subject together in a single parenteral dosage composition such as in a saline solution or other physiologically acceptable solution, or each agent administered in separate parenteral dosage formulations. Where separate dosage formulations are used, the compositions comprising an antibody or antigen-binding fragment and one or more additional active agents can be administered at essentially the same time, i.e., concurrently, or at separately staggered times, i.e., sequentially and in any order; combination therapy is understood to include all these regimens.

In some embodiments, an antibody (or one or more nucleic acid, host cell, vector, or composition) is administered to a subject who has previously received one or more anti-inflammatory agent and/or one or more antiviral agent. In some embodiments, the antiviral is a neuramidase inhibitor (NAI), such as, for example, oseltamivir, zanamivir, peramivir, or laninamivir. In some embodiments, one or more anti-inflammatory agent and/or one or more antiviral agent is administered to a subject who has previously received an antibody (or one or more nucleic acid, host cell, vector, or composition). In some embodiments, the antiviral is a neuramidase inhibitor (NAI), such as, for example, oseltamivir, zanamivir, peramivir, or laninamivir.

In a related aspect, uses of the presently disclosed antibodies, antigen-binding fragments, vectors, host cells, and compositions (e.g., in the diagnosis, prophylaxis, and/or treatment of an influenza infection, in the manufacture of a medicament for preventing or treating an influenza infection) are provided.

In certain embodiments, an antibody, antigen-binding fragment, polynucleotide, vector, host cell, or composition is provided for use in a method of treating or preventing an influenza infection in a subject.

In certain embodiments, an antibody, antigen-binding fragment, or composition is provided for use in a method of manufacturing or preparing a medicament for treating or preventing an influenza infection in a subject.

The present disclosure also provides the following non-limiting enumerated Embodiments.

Embodiment 1. An anti-influenza neuraminidase (anti-NA) antibody, or an antigen-binding fragment thereof, comprising (i) a heavy chain variable domain (VH) comprising a complementarity determining region (CDR)H1, a CDRH2, and a CDRH3, and (ii) and a light chain variable domain (VL) comprising a CDRL1, a CDRL2, and a CDRL3, wherein: (a) the CDRH1 comprises or consists of the amino acid sequence set forth in SEQ ID NO:55, SEQ ID NO.: 3, SEQ ID NO:47, or SEQ ID NO.: 49; and/or (b) the CDRH2 comprises or consists of the amino acid sequence set forth in SEQ ID NO:4, SEQ ID NO:57, or SEQ ID NO:61; and/or (c) the CDRH3 comprises or consists of the amino acid sequence set forth in SEQ ID NO:5, SEQ ID NO:15, SEQ ID NO:51, or SEQ ID NO:53, (d) the CDRL1 comprises or consists of the amino acid sequence set forth in SEQ ID NO.9 or SEQ ID NO:32; and/or (e) the CDRL2 comprises or consists of the amino acid sequence set forth in SEQ ID NO.: 10; and/or (f) the CDRL3 comprises or consists of the amino acid sequence set forth in SEQ ID NO:11, 18, 21, 24, 33, or 67, optionally provided that the CDRH1, the CDRH2, the CDRH3, the CDRL1, the CDRL2, and the CDRL3 do not comprise or consist of the amino acid sequences set forth in SEQ ID NOs: (i) 3-5 and 9-11, respectively; (ii) 3, 4, 15 and 9-11, respectively; (iii) 3-5, 9, 10, and 18, respectively; (iv) 3-5, 9, 10, and 21, respectively; (v) 3-5, 9-11, and 24, respectively; or (vi) 3-5, 32, 96, and 33, respectively.

Embodiment 2. The antibody or antigen-binding fragment of Embodiment 1, wherein the CDRH3 and CDRL3 comprise or consist of the amino acid sequences set forth in SEQ ID NOs: (i) 5 and 11, respectively; (ii) 5 and 18, respectively; (iii) 5 and 21, respectively; (iv) 5 and 24, respectively; (v) 5 and 33, respectively; (vi) 5 and 67, respectively; (vii) 15 and 11, respectively; (viii) 15 and 18, respectively; (ix) 15 and 21, respectively; (x) 15 and 24, respectively; (xi) 15 and 33, respectively; (xii) 15 and 67, respectively; (xiii) 51 and 11, respectively; (xiv) 51 and 18, respectively; (xv) 51 and 21, respectively; (xvi) 51 and 24, respectively; (xvii) 51 and 33, respectively; (xviii) 51 and 67, respectively; (xix) 53 and 11, respectively; (xx) 53 and 18, respectively; (xxi) 53 and 21, respectively; (xxii) 53 and 24, respectively; (xxiii) 53 and 33, respectively; or (xxiv) 53 and 67, respectively.

Embodiment 3. The antibody or antigen-binding fragment of Embodiment 1 or 2, wherein the CDRH1, CDRH2, CDRH3, CDRL1, CDRL2, and CDRL3 comprise or consist of the amino acid sequences set forth in SEQ ID NOs: (i) 55, 4, 5, and 9-11, respectively; (ii) 47, 4, 5, and 9-11, respectively; (iii) 47, 4, 5, 9, 10, and 67, respectively; (iv) 49, 4, 5, and 9-11, respectively; (v) 47, 4, 5, 9, 10, and 67, respectively; (vi) 3-5, 9, 10, and 67, respectively; (vii) 55, 4, 5, 9, 10, and 67, respectively; (viii) 3, 4, 51, and 9-11, respectively; (ix) 3, 4, 51, 9, 10, and 67, respectively; (x) 55, 4, 5, and 9-11, respectively; (xi) 55, 4, 5, 9, 10, and 67, respectively; (xii) 3, 61, 5, and 9-11, respectively; (xiii) 3, 61, 5, 9, 10, and 67 respectively; (xiv) 3-5 and 9-11, respectively; or (xv) 3, 57, 5, and 9-11, respectively.

Embodiment 3a. The antibody or antigen-binding fragment of Embodiment 1 or 2, wherein the CDRH1, CDRH2, CDRH3, CDRL1, CDRL2, and CDRL3 amino acid sequences are as set forth in SEQ ID NOs: (i) 55, 4, 5, and 9-11, respectively; (ii) 47, 4, 5, and 9-11, respectively; (iii) 47, 4, 5, 9, 10, and 67, respectively; (iv) 49, 4, 5, and 9-11, respectively; (v) 47, 4, 5, 9, 10, and 67, respectively; (vi) 3-5, 9, 10, and 67, respectively; (vii) 55, 4, 5, 9, 10, and 67, respectively; (viii) 3, 4, 51, and 9-11, respectively; (ix) 3, 4, 51, 9, 10, and 67, respectively; (x) 55, 4, 5, and 9-11, respectively; (xi) 55, 4, 5, 9, 10, and 67, respectively; (xii) 3, 61, 5, and 9-11, respectively; (xiii) 3, 61, 5, 9, 10, and 67 respectively; (xiv) 3-5 and 9-11, respectively; or (xv) 3, 57, 5, and 9-11, respectively.

Embodiment 4. An anti-influenza neuraminidase (anti-NA) antibody, or an antigen-binding fragment thereof, comprising (i) a heavy chain variable domain (VH) and (ii) light chain variable domain (VL), wherein: (i) the VH comprises the CDRH1, CDRH2, and CDRH3 of the VH amino acid sequence set forth in SEQ ID NO:54, and the VL comprises the CDRL1, CDRL2, and CDRL3 of the VL amino acid sequence set forth in SEQ ID NO:8; (ii) the VH comprises the CDRH1, CDRH2, and CDRH3 of the VH amino acid sequence set forth in SEQ ID NO:46, and the VL comprises the CDRL1, CDRL2, and CDRL3 of the VL amino acid sequence set forth in SEQ ID NO:8; (iii) the VH comprises the CDRH1, CDRH2, and CDRH3 of the VH amino acid sequence set forth in SEQ ID NO:46, and the VL comprises the CDRL1, CDRL2, and CDRL3 of the VL amino acid sequence set forth in SEQ ID NO:17; (iv) the VH comprises the CDRH1, CDRH2, and CDRH3 of the VH amino acid sequence set forth in SEQ ID NO:46, and the VL comprises the CDRL1, CDRL2, and CDRL3 of the VL amino acid sequence set forth in SEQ ID NO:20; (v) the VH comprises the CDRH1, CDRH2, and CDRH3 of the VH amino acid sequence set forth in SEQ ID NO:46, and the VL comprises the CDRL1, CDRL2, and CDRL3 of the VL amino acid sequence set forth in SEQ ID NO:23; (vi) the VH comprises the CDRH1, CDRH2, and CDRH3 of the VH amino acid sequence set forth in SEQ ID NO:46, and the VL comprises the CDRL1, CDRL2, and CDRL3 of the VL amino acid sequence set forth in SEQ ID NO:31; (vii) the VH comprises the CDRH1, CDRH2, and CDRH3 of the VH amino acid sequence set forth in SEQ ID NO:46, and the VL comprises the CDRL1, CDRL2, and CDRL3 of the VL amino acid sequence set forth in SEQ ID NO:66; (viii) the VH comprises the CDRH1, CDRH2, and CDRH3 of the VH amino acid sequence set forth in SEQ ID NO:48, and the VL comprises the CDRL1, CDRL2, and CDRL3 of the VL amino acid sequence set forth in SEQ ID NO:8; (ix) the VH comprises the CDRH1, CDRH2, and CDRH3 of the VH amino acid sequence set forth in SEQ ID NO:48, and the VL comprises the CDRL1, CDRL2, and CDRL3 of the VL amino acid sequence set forth in SEQ ID NO:17; (x) the VH comprises the CDRH1, CDRH2, and CDRH3 of the VH amino acid sequence set forth in SEQ ID NO:48, and the VL comprises the CDRL1, CDRL2, and CDRL3 of the VL amino acid sequence set forth in SEQ ID NO:20; (xi) the VH comprises the CDRH1, CDRH2, and CDRH3 of the VH amino acid sequence set forth in SEQ ID NO:48, and the VL comprises the CDRL1, CDRL2, and CDRL3 of the VL amino acid sequence set forth in SEQ ID NO:23; (xii) the VH comprises the CDRH1, CDRH2, and CDRH3 of the VH amino acid sequence set forth in SEQ ID NO:48, and the VL comprises the CDRL1, CDRL2, and CDRL3 of the VL amino acid sequence set forth in SEQ ID NO:31; (xiii) the VH comprises the CDRH1, CDRH2, and CDRH3 of the VH amino acid sequence set forth in SEQ ID NO:48, and the VL comprises the CDRL1, CDRL2, and CDRL3 of the VL amino acid sequence set forth in SEQ ID NO:66; (xiv) the VH comprises the complementarity determining region (CDR)H1, CDRH2, and CDRH3 of the VH amino acid sequence set forth in SEQ ID NO:2, and the VL comprises the CDRL1, CDRL2, and CDRL3 of the VL amino acid sequence set forth in SEQ ID NO:66; (xv) the VH comprises the CDRH1, CDRH2, and CDRH3 of the VH amino acid sequence set forth in SEQ ID NO:54, and the VL comprises the CDRL1, CDRL2, and CDRL3 of the VL amino acid sequence set forth in SEQ ID NO:17; (xvi) the VH comprises the CDRH1, CDRH2, and CDRH3 of the VH amino acid sequence set forth in SEQ ID NO:54, and the VL comprises the CDRL1, CDRL2, and CDRL3 of the VL amino acid sequence set forth in SEQ ID NO:20; (xvii) the VH comprises the CDRH1, CDRH2, and CDRH3 of the VH amino acid sequence set forth in SEQ ID NO:54, and the VL comprises the CDRL1, CDRL2, and CDRL3 of the VL amino acid sequence set forth in SEQ ID NO:23; (xviii) the VH comprises the CDRH1, CDRH2, and CDRH3 of the VH amino acid sequence set forth in SEQ ID NO:54, and the VL comprises the CDRL1, CDRL2, and CDRL3 of the VL amino acid sequence set forth in SEQ ID NO:31; (xix) the VH comprises the CDRH1, CDRH2, and CDRH3 of the VH amino acid sequence set forth in SEQ ID NO:54, and the VL comprises the CDRL1, CDRL2, and CDRL3 of the VL amino acid sequence set forth in SEQ ID NO:66; (xx) the VH comprises the CDRH1, CDRH2, and CDRH3 of the VH amino acid sequence set forth in SEQ ID NO:56, and the VL comprises the CDRL1, CDRL2, and CDRL3 of the VL amino acid sequence set forth in SEQ ID NO:8; (xxi) the VH comprises the CDRH1, CDRH2, and CDRH3 of the VH amino acid sequence set forth in SEQ ID NO:56, and the VL comprises the CDRL1, CDRL2, and CDRL3 of the VL amino acid sequence set forth in SEQ ID NO:17; (xxii) the VH comprises the CDRH1, CDRH2, and CDRH3 of the VH amino acid sequence set forth in SEQ ID NO:56, and the VL comprises the CDRL1, CDRL2, and CDRL3 of the VL amino acid sequence set forth in SEQ ID NO:20; (xxiii) the VH comprises the CDRH1, CDRH2, and CDRH3 of the VH amino acid sequence set forth in SEQ ID NO:56, and the VL comprises the CDRL1, CDRL2, and CDRL3 of the VL amino acid sequence set forth in SEQ ID NO:23; (xxiv) the VH comprises the CDRH1, CDRH2, and CDRH3 of the VH amino acid sequence set forth in SEQ ID NO:54, and the VL comprises the CDRL1, CDRL2, and CDRL3 of the VL amino acid sequence set forth in SEQ ID NO:31; (xxv) the VH comprises the CDRH1, CDRH2, and CDRH3 of the VH amino acid sequence set forth in SEQ ID NO:54, and the VL comprises the CDRL1, CDRL2, and CDRL3 of the VL amino acid sequence set forth in SEQ ID NO:66; (xxvi) the VH comprises the CDRH1, CDRH2, and CDRH3 of the VH amino acid sequence set forth in SEQ ID NO:60, and the VL comprises the CDRL1, CDRL2, and CDRL3 of the VL amino acid sequence set forth in SEQ ID NO:8; (xxvii) the VH comprises the CDRH1, CDRH2, and CDRH3 of the VH amino acid sequence set forth in SEQ ID NO:60, and the VL comprises the CDRL1, CDRL2, and CDRL3 of the VL amino acid sequence set forth in SEQ ID NO:17; (xxviii) the VH comprises the CDRH1, CDRH2, and CDRH3 of the VH amino acid sequence set forth in SEQ ID NO:60, and the VL comprises the CDRL1, CDRL2, and CDRL3 of the VL amino acid sequence set forth in SEQ ID NO:20; (xxix) the VH comprises the CDRH1, CDRH2, and CDRH3 of the VH amino acid sequence set forth in SEQ ID NO:60, and the VL comprises the CDRL1, CDRL2, and CDRL3 of the VL amino acid sequence set forth in SEQ ID NO:23; (xxx) the VH comprises the CDRH1, CDRH2, and CDRH3 of the VH amino acid sequence set forth in SEQ ID NO:60, and the VL comprises the CDRL1, CDRL2, and CDRL3 of the VL amino acid sequence set forth in SEQ ID NO:31; (xxxi) the VH comprises the CDRH1, CDRH2, and CDRH3 of the VH amino acid sequence set forth in SEQ ID NO:60, and the VL comprises the CDRL1, CDRL2, and CDRL3 of the VL amino acid sequence set forth in SEQ ID NO:66; (xxxii) the VH comprises the CDRH1, CDRH2, and CDRH3 of the VH amino acid sequence set forth in SEQ ID NO:50, and the VL comprises the CDRL1, CDRL2, and CDRL3 of the VL amino acid sequence set forth in SEQ ID NO:8; (xxxiii) the VH comprises the CDRH1, CDRH2, and CDRH3 of the VH amino acid sequence set forth in SEQ ID NO:50, and the VL comprises the CDRL1, CDRL2, and CDRL3 of the VL amino acid sequence set forth in SEQ ID NO:17; (xxxiv) the VH comprises the CDRH1, CDRH2, and CDRH3 of the VH amino acid sequence set forth in SEQ ID NO:50, and the VL comprises the CDRL1, CDRL2, and CDRL3 of the VL amino acid sequence set forth in SEQ ID NO:20; (xxxv) the VH comprises the CDRH1, CDRH2, and CDRH3 of the VH amino acid sequence set forth in SEQ ID NO:50, and the VL comprises the CDRL1, CDRL2, and CDRL3 of the VL amino acid sequence set forth in SEQ ID NO:23; (xxxvi) the VH comprises the CDRH1, CDRH2, and CDRH3 of the VH amino acid sequence set forth in SEQ ID NO:50, and the VL comprises the CDRL1, CDRL2, and CDRL3 of the VL amino acid sequence set forth in SEQ ID NO:31; (xxxvii) the VH comprises the CDRH1, CDRH2, and CDRH3 of the VH amino acid sequence set forth in SEQ ID NO:50, and the VL comprises the CDRL1, CDRL2, and CDRL3 of the VL amino acid sequence set forth in SEQ ID NO:66; (xxxviii) the VH comprises the CDRH1, CDRH2, and CDRH3 of the VH amino acid sequence set forth in SEQ ID NO:52, and the VL comprises the CDRL1, CDRL2, and CDRL3 of the VL amino acid sequence set forth in SEQ ID NO:8; (xxxix) the VH comprises the CDRH1, CDRH2, and CDRH3 of the VH amino acid sequence set forth in SEQ ID NO:52, and the VL comprises the CDRL1, CDRL2, and CDRL3 of the VL amino acid sequence set forth in SEQ ID NO.: 17; (xxxx) the VH comprises the CDRH1, CDRH2, and CDRH3 of the VH amino acid sequence set forth in SEQ ID NO:52, and the VL comprises the CDRL1, CDRL2, and CDRL3 of the VL amino acid sequence set forth in SEQ ID NO:20; (xxxxi) the VH comprises the CDRH1, CDRH2, and CDRH3 of the VH amino acid sequence set forth in SEQ ID NO:52, and the VL comprises the CDRL1, CDRL2, and CDRL3 of the VL amino acid sequence set forth in SEQ ID NO:23; (xxxxii) the VH comprises the CDRH1, CDRH2, and CDRH3 of the VH amino acid sequence set forth in SEQ ID NO:52, and the VL comprises the CDRL1, CDRL2, and CDRL3 of the VL amino acid sequence set forth in SEQ ID NO:31; (xxxxiii) the VH comprises the CDRH1, CDRH2, and CDRH3 of the VH amino acid sequence set forth in SEQ ID NO:52, and the VL comprises the CDRL1, CDRL2, and CDRL3 of the VL amino acid sequence set forth in SEQ ID NO:66; (xxxxiv) the VH comprises the CDRH1, CDRH2, and CDRH3 of the VH amino acid sequence set forth in SEQ ID NO:2, and the VL comprises the CDRL1, CDRL2, and CDRL3 of the VL amino acid sequence set forth in SEQ ID NO:8, or (xxxxv) the VH comprises the CDRH1, CDRH2, and CDRH3 of the VH amino acid sequence set forth in SEQ ID NO:65, and the VL comprises the CDRL1, CDRL2, and CDRL3 of the VL amino acid sequence set forth in SEQ ID NO:68, wherein CDRH1, CDRH2, CDRH3, CDRL1, CDRL2, and CDRL3 are optionally defined in accordance with the IMGT numbering system.

Embodiment 5. The antibody or antigen-binding fragment of any one of Embodiments 1-4, wherein: (i) the VH comprises or consists of amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity (or similarity) to, or comprises or consists of, the amino acid sequence set forth in any one of SEQ ID NOs: 54, 2, 14, 45, 46, 48, 50, 52, 56, 58, 60, 63, and 65; and (ii) the VL comprises or consists of amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity (or similarity) to, or comprises or consists of, the amino acid sequence set forth in any one of SEQ ID NOs:8, 17, 20, 23, 31, 37, 66, and 68, optionally provided that the VH and VL do not comprise or consist of the amino acid sequences set forth in: (a) 2 and 8, respectively; (b) 14 and 8, respectively; (c) 2 and 17, respectively; (d) 2 and 20, respectively; (e) 2 and 23, respectively; or (f) 2 and 31, respectively.

Embodiment 6. The antibody or antigen-binding fragment of any one of Embodiments 1-5, wherein the VH and VL have at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity (or similarity) to, or comprise or consist of, the amino acid sequences set forth in SEQ ID NOs: (i) 54 and 8, respectively; (ii) 46 and 8, respectively; (iii) 46 and 17, respectively; (iv) 46 and 20, respectively; (v) 46 and 23, respectively; (vi) 46 and 31, respectively; (vii) 46 and 66, respectively; (viii) 48 and 8, respectively; (ix) 48 and 17, respectively; (x) 48 and 20, respectively; (xi) 48 and 23, respectively; (xii) 48 and 31, respectively; (xiii) 48 and 66, respectively; (xiv) 2 and 66, respectively; (xv) 54 and 17, respectively; (xvi) 54 and 20, respectively; (xvii) 54 and 23, respectively; (xviii) 54 and 31; (xix) 54 and 66, respectively; (xx) 56 and 8, respectively; (xxi) 56 and 17, respectively; (xxii) 56 and 20, respectively; (xxiii) 56 and 23, respectively; (xxiv) 54 and 31, respectively; (xxv) 54 and 66, respectively; (xxvi) 60 and 8, respectively; (xxvii) 60 and 17, respectively; (xxviii) 60 and 20, respectively; (xxix) 60 and 23, respectively; (xxx) 60 and 31, respectively; (xxxi) 60 and 66, respectively; (xxxii) 2, 14, 45, 46, 48, 50, 52, 54, 56, 58, 60, 63, or 65, and 37, respectively; (xxxiii) 2, 14, 45, 46, 48, 50, 52, 54, 56, 58, 60, 63, or 65, and 68, respectively; (xxxiv) 45, 46, 48, 50, 52, 54, 56, 58, 60, 63, or 65, and 8, respectively; (xxxv) 45, 46, 48, 50, 52, 54, 56, 58, 60, 63, or 65, and 17, respectively; (xxxvi) 45, 46, 48, 50, 52, 54, 56, 58, 60, 63, or 65, and 20, respectively; (xxxvii) 45, 46, 48, 50, 52, 54, 56, 58, 60, 63, or 65, and 23, respectively; (xxxviii) 45, 46, 48, 50, 52, 54, 56, 58, 60, 63, or 65, and 31, respectively; (xxxix) 45, 46, 48, 50, 52, 54, 56, 58, 60, 63, or 65, and 37, respectively; (xxxx) 45, 46, 48, 50, 52, 54, 56, 58, 60, 63, or 65, and 66, respectively; (xxxxi) 45, 46, 48, 50, 52, 54, 56, 58, 60, 63, or 65, and 68, respectively; (xxxxii) 2, 14, 45, 46, 48, 50, 52, 54, 56, 58, 60, 63, or 65, and 66, respectively; (xxxxiii) 2 and 8, respectively; (xxxxiv) 2 and 37, respectively; or (xxxxv) 65 and 68, respectively.

Embodiment 7. The antibody or antigen-binding fragment of any one of Embodiments 1-6, wherein the VH and VL comprise or consist of the amino acid sequences set forth in SEQ ID NOs: (i) 54 and 8, respectively; (ii) 46 and 37, respectively; (iii) 48 and 37, respectively; (iv) 50 and 37, respectively; (v) 52 and 37, respectively; (vi) 54 and 37, respectively; (vii) 56 and 37, respectively; (viii) 58 and 37, respectively; (ix) 60 and 37, respectively; (x) 63 and 37, respectively; (xi) 65 and 37, respectively; (xii) 45 and 66, respectively; (xiii) 46 and 66, respectively; (xiv) 48 and 66, respectively; (xv) 50 and 66, respectively; (xvi) 52 and 66, respectively; (xvii) 54 and 66, respectively; (xviii) 56 and 66, respectively; (xix) 58 and 66, respectively; (xx) 60 and 66, respectively; (xxi) 63 and 66, respectively; (xxii) 65 and 66, respectively; (xxiii) 45 and 68, respectively; (xxiv) 46 and 68, respectively; (xxv) 48 and 68, respectively; (xxvi) 50 and 68, respectively; (xxvii) 52 and 68, respectively; (xxviii) 54 and 68, respectively; (xxix) 56 and 68, respectively; (xxx) 58 and 68, respectively; (xxxi) 60 and 68, respectively; (xxxii) 63 and 68, respectively; (xxxiii) 65 and 68, respectively; (xxxiv) 45, 46, 48, 50, 52, 54, 56, 58, 60, 63, or 65, and 8, respectively; (xxxv) 45, 46, 48, 50, 52, 54, 56, 58, 60, 63, or 65, and 17, respectively; (xxxvi) 45, 46, 48, 50, 52, 54, 56, 58, 60, 63, or 65, and 20, respectively; (xxxvii) 45, 46, 48, 50, 52, 54, 56, 58, 60, 63, or 65, and 23, respectively; (xxxviii) 45, 46, 48, 50, 52, 54, 56, 58, 60, 63, or 65, and 31, respectively; (xxxix) 2, 45, 46, 48, 50, 52, 54, 56, 58, 60, 63, or 65, and 37, respectively; (xxxx) 2, 45, 46, 48, 50, 52, 54, 56, 58, 60, 63, or 65, and 66, respectively; (xxxxi) 2, 45, 46, 48, 50, 52, 54, 56, 58, 60, 63, or 65, and 68, respectively; (xxxxii) 2, 14, 45, 46, 48, 50, 52, 54, 56, 58, 60, 63, or 65, and 66, respectively; (xxxxiii) 2 and 8, respectively; (xxxxiv) 2 and 37, respectively; (xxxxv) 45 and 37, respectively; (xxxxvi) 46 and 8, respectively; or (xxxxvii) 56 and 8, respectively.

Embodiment 7a. The antibody or antigen-binding fragment of any one of Embodiments 1-6, wherein the VH and VL comprise or consist of the amino acid sequences set forth in SEQ ID NOs: (i) 54 and 8, respectively.

Embodiment 8. An anti-influenza neuraminidase (anti-NA) antibody, or an antigen-binding fragment thereof, comprising (i) a heavy chain variable domain (VH) comprising a complementarity determining region (CDR)H1, a CDRH2, and a CDRH3, and (ii) and a light chain variable domain (VL) comprising a CDRL1, a CDRL2, and a CDRL3, wherein: (a) CDRH2 is as set forth in SEQ ID NO.4 and is comprised in SEQ ID NO:59, CDRH1 is as set forth in SEQ ID NO:3, and CDRH3 is as set forth in SEQ ID NO:5; (b) CDRH2 is as set forth in SEQ ID NO:61 and is comprised in SEQ ID NO:62, CDRH1 is as set forth in SEQ ID NO:3, and CDRH3 is as set forth in SEQ ID NO:5; or (c) CDRH2 is as set forth in SEQ ID NO:61 and is comprised in SEQ ID NO:64, CDRH1 is as set forth in SEQ ID NO:3, and CDRH3 is as set forth in SEQ ID NO.: 5.

Embodiment 9. The antibody or antigen-binding fragment of Embodiment 8, wherein CDRL1 is as set forth in SEQ ID NO.: 9, CDRL2 is as set forth in SEQ ID NO:10, and CDRL3 is as set forth in any one of SEQ ID NOs:11, 18, 21, 24, 33, and 67.

Embodiment 10. The antibody or antigen-binding fragment of Embodiment 8, wherein CDRL1 is as set forth in SEQ ID NO:32, CDRL2 is as set forth in SEQ ID NO:10 or 96, and CDRL3 is as set forth in any one of SEQ ID NOs:11, 18, 21, 24, 33, and 67.

Embodiment 11. An anti-influenza neuraminidase (anti-NA) antibody, or an antigen-binding fragment thereof, comprising a heavy chain variable domain (VH) and a light chain variable domain (VL), wherein the VH and VL comprise or consist of the amino acid sequences set forth in SEQ ID NOs: (i) 54 and 8, respectively; (ii) 46 and 8, respectively; (iii) 48 and 8, respectively; (iv) 50 and 8, respectively; (v) 52 and 8, respectively; (vi) 65 and 68, respectively; (vii) 56 and 8, respectively; (viii) 58 and 8, respectively; (ix) 60 and 8, respectively; (x) 63 and 8, respectively; (xi) 46 and 66, respectively; (xii) 48 and 66, respectively; (xiii) 50 and 66, respectively; (xiv) 52 and 66, respectively; (xv) 54 and 66, respectively; (xvi) 56 and 66, respectively; (xvii) 58 and 66, respectively; (xviii) 60 and 66, respectively; (xix) 63 and 66, respectively; or (xx) 2 and 37, respectively.

Embodiment 12. The antibody or antigen-binding fragment of any one of Embodiments 1-11, wherein the influenza comprises an influenza A virus, an influenza B virus, or both.

Embodiment 13. The antibody or antigen-binding fragment of any one of Embodiments 1-12, wherein the antibody, or the antigen-binding fragment, comprises a human antibody, a monoclonal antibody, a purified antibody, a single chain antibody, a Fab, a Fab′, a F(ab′)2, or Fv.

Embodiment 14. The antibody or antigen-binding fragment of any one of Embodiments 1-13, wherein the antibody or antigen-binding fragment is a multi-specific antibody or antigen-binding fragment, wherein, optionally, the antibody or antigen-binding fragment is a bi-specific antibody or antigen-binding fragment.

Embodiment 15. The antibody or antigen-binding fragment of any one of Embodiments 1-14, wherein the antibody or antigen-binding fragment comprises an (e.g., IgG1) Fc polypeptide or a fragment thereof.

Embodiment 16. The antibody or antigen-binding fragment of any one of Embodiments 1-15, which comprises a IgG, IgA, IgM, IgE, or IgD isotype.

Embodiment 17. The antibody or antigen-binding fragment of any one of Embodiments 1-16, which comprises an IgG isotype selected from IgG1, IgG2, IgG3, and IgG4, optionally with a C-terminal lysine removed or a C-terminal glycine-lysine removed.

Embodiment 18. The antibody or antigen-binding fragment of any one of Embodiments 1-17, which comprises an IgG1 isotype.

Embodiment 19. The antibody or antigen-binding fragment of any one of Embodiments 1-18, which comprises an IgG1m3 allotype, an IgG1m17 allotype, an IgG1 ml allotype, or any combination thereof.

Embodiment 20. The antibody or antigen-binding fragment of Embodiment 15-19, wherein the Fc polypeptide or fragment thereof comprises: (i) a mutation that increases binding affinity to a human FcRn (e.g., as measured using surface plasmon resonance (SPR) (e.g., Biacore, e.g., T200 instrument, using manufacturer's protocols)), as compared to a reference Fc polypeptide that does not comprise the mutation; and/or (ii) a mutation that increases binding affinity to a human FcγR (e.g., as measured using surface plasmon resonance (SPR) (e.g., Biacore, e.g., T200 instrument, using manufacturer's protocols, and/or as measured using mesoscale discovery (MSD))) as compared to a reference Fc polypeptide that does not comprise the mutation.

Embodiment 21. The antibody or antigen-binding fragment of Embodiment 20, wherein the mutation that increases binding affinity to a human FcRn comprises: M428L; N434S; N434H; N434A; N434S; M252Y; S254T; T256E; T250Q; P257I; Q311I; D376V; T307A; E380A; or any combination thereof.

Embodiment 22. The antibody or antigen-binding fragment of Embodiment 20 or 21, wherein the mutation that increases binding affinity to a human FcRn comprises: (i) M428L/N434S; (ii) M252Y/S254T/T256E; (iii) T250Q/M428L; (iv) P257I/Q311I; (v) P257I/N434H; (vi) D376V/N434H; (vii) T307A/E380A/N434A; (viii) M428L/N434A; or (ix) any combination of (i)-(viii).

Embodiment 23. The antibody or antigen-binding fragment of any one of Embodiments 20-22, wherein the mutation that increases binding affinity to a human FcRn comprises M428L/N434S or M428L/N434A.

Embodiment 24. The antibody or antigen-binding fragment of any one of Embodiments 20-23, wherein the mutation that enhances binding to a FcγR comprises S239D; I332E; A330L; G236A; or any combination thereof.

Embodiment 25. The antibody or antigen-binding fragment of any one of Embodiments 20-24, wherein the mutation that enhances binding to a FcγR comprises: (i) S239D/I332E; (ii) S239D/A330L/I332E; (iii) G236A/S239D/I332E; or (iv) G236A/A330L/I332E, wherein the Fc polypeptide or fragment thereof optionally comprises Ser at position 239.

Embodiment 26. The antibody or antigen-binding fragment of any one of Embodiments 1-25, which comprises a mutation that alters glycosylation, wherein the mutation that alters glycosylation comprises N297A, N297Q, or N297G, and/or which is aglycosylated, and/or which is afucosylated.

Embodiment 27. An anti-influenza neuraminidase (anti-NA) antibody, or an antigen-binding fragment thereof, comprising (i) in a heavy chain, (i)(a) a variable domain (VH) comprising the complementarity determining region (CDR)H1, CDRH2, and CDRH3 amino acid sequences set forth in SEQ ID NOs:3-5, respectively, and (i)(b) the mutations M428L and N434A; and (ii) in a light chain, a light chain variable domain (VL) comprising the CDRL1, CDRL2, and CDRL3 amino acid sequences set forth in SEQ ID NOs:9-11, respectively.

Embodiment 28. An anti-influenza neuraminidase (anti-NA) antibody, or an antigen-binding fragment thereof, comprising (i) in a heavy chain, (i)(a) a variable domain (VH) comprising the complementarity determining region (CDR)H1, CDRH2, and CDRH3 amino acid sequences set forth in SEQ ID NOs:3-5, respectively, and (i)(b) the mutations M428L and N434S; and (ii) in a light chain, a light chain variable domain (VL) comprising the CDRL1, CDRL2, and CDRL3 amino acid sequences set forth in SEQ ID NOs:9-11, respectively.

Embodiment 29. An anti-influenza neuraminidase (anti-NA) antibody, or an antigen-binding fragment thereof, comprising (i) in a heavy chain, (i)(a) a variable domain (VH) comprising the complementarity determining region (CDR)H1, CDRH2, and CDRH3 amino acid sequences set forth in SEQ ID NOs:55, 4, and 5, respectively, wherein, optionally, the VH comprises an amino acid sequence having at least 90%, at least 91%, at least 92%, at least 93%, at least 94%, at least 95%, at least 96%, at least 97%, at least 98%, or at least 99% identity to, or consisting of, the amino acid sequence set forth in SEQ ID NO:54, and (i)(b) the mutations M428L and N434S; and (ii) in a light chain, a light chain variable domain (VL) comprising the CDRL1, CDRL2, and CDRL3 amino acid sequences set forth in SEQ ID NOs:9-11, respectively, wherein, optionally, the VL comprises an amino acid sequence having at least 90%, at least 91%, at least 92%, at least 93%, at least 94%, at least 95%, at least 96%, at least 97%, at least 98%, or at least 99% identity (or similarity) to, or consisting of, the amino acid sequence set forth in SEQ ID NO.: 8.

Embodiment 30. An anti-influenza neuraminidase (anti-NA) antibody, or an antigen-binding fragment thereof, comprising (i) in a heavy chain, (i)(a) a variable domain (VH) comprising the complementarity determining region (CDR)H1, CDRH2, and CDRH3 amino acid sequences set forth in SEQ ID NOs:47, 4, and 5, respectively, wherein, optionally, the VH comprises an amino acid sequence having at least 90%, at least 91%, at least 92%, at least 93%, at least 94%, at least 95%, at least 96%, at least 97%, at least 98%, or at least 99% identity (or similarity) to, or consisting of, the amino acid sequence set forth in SEQ ID NO:46, and (i)(b) the mutations M428L and N434S; and (ii) in a light chain, a light chain variable domain (VL) comprising the CDRL1, CDRL2, and CDRL3 amino acid sequences set forth in SEQ ID NOs:9-11, respectively, wherein, optionally, the VL comprises an amino acid sequence having at least 90%, at least 91%, at least 92%, at least 93%, at least 94%, at least 95%, at least 96%, at least 97%, at least 98%, or at least 99% identity (or similarity) to, or consisting of, the amino acid sequence set forth in SEQ ID NO:8.

Embodiment 31. An anti-influenza neuraminidase (anti-NA) antibody, or an antigen-binding fragment thereof, comprising (i) in a heavy chain, (i)(a) a variable domain (VH) comprising the complementarity determining region (CDR)H1, CDRH2, and CDRH3 amino acid sequences set forth in SEQ ID NOs:3-5, respectively, wherein, optionally, the VH comprises an amino acid sequence having at least 90%, at least 91%, at least 92%, at least 93%, at least 94%, at least 95%, at least 96%, at least 97%, at least 98%, or at least 99% identity (or similarity) to, or consisting of, the amino acid sequence set forth in SEQ ID NO:65, and (i)(b) the mutations M428L and N434S; and (ii) in a light chain, a light chain variable domain (VL) comprising the CDRL1, CDRL2, and CDRL3 amino acid sequences set forth in SEQ ID NOs:9-11, respectively, wherein, optionally, the VL comprises an amino acid sequence having at least 90%, at least 91%, at least 92%, at least 93%, at least 94%, at least 95%, at least 96%, at least 97%, at least 98%, or at least 99% identity (or similarity) to, or consisting of, the amino acid sequence set forth in SEQ ID NO:68.

Embodiment 32. An anti-influenza neuraminidase (anti-NA) antibody, or an antigen-binding fragment thereof, comprising (i) in a heavy chain, (i)(a) a variable domain (VH) comprising the complementarity determining region (CDR)H1, CDRH2, and CDRH3 amino acid sequences set forth in SEQ ID NOs:3, 57, and 5, respectively, wherein, optionally, the VH comprises an amino acid sequence having at least 90%, at least 91%, at least 92%, at least 93%, at least 94%, at least 95%, at least 96%, at least 97%, at least 98%, or at least 99% identity (or similarity) to, or consisting of, the amino acid sequence set forth in SEQ ID NO:56, and (i)(b) the mutations M428L and N434S; and (ii) in a light chain, a light chain variable domain (VL) comprising the CDRL1, CDRL2, and CDRL3 amino acid sequences set forth in SEQ ID NOs:9-11, respectively, wherein, optionally, the VL comprises an amino acid sequence having at least 90%, at least 91%, at least 92%, at least 93%, at least 94%, at least 95%, at least 96%, at least 97%, at least 98%, or at least 99% identity (or similarity) to, or consisting of, the amino acid sequence set forth in SEQ ID NO:8.

Embodiment 33. The antibody or antigen-binding fragment of any one of Embodiments 27-32, wherein VH and VL have at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity (or similarity) to, or comprise or consist of, the amino acid sequences set forth in SEQ ID NOs: (i) 54 and 8, respectively; (ii) 2 and 31, respectively; (iii) 2 and 8, respectively; (iv) 46 and 8, respectively; (v) 65 and 68, respectively; or (vi) 56 and 8, respectively.

Embodiment 34. The antibody or antigen-binding fragment of any one of Embodiments 27-33, wherein VH and VL comprise or consist of, the amino acid sequences set forth in SEQ ID NOs: (i) 54 and 8, respectively; (ii) 2 and 31, respectively; (iii) 2 and 8, respectively; (iv) 46 and 8, respectively; (v) 65 and 68, respectively; or (vi) 56 and 8, respectively.

Embodiment 35. The antibody or antigen-binding fragment of any one of Embodiments 27-34, wherein VH and VL have at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity (or similarity) to, or comprise or consist of, the amino acid sequences set forth in SEQ ID NOs:2 and 37, respectively.

Embodiment 36. The antibody or antigen-binding fragment of any one of Embodiments 27-33 and 31, wherein VH and VL comprise or consist of, the amino acid sequences set forth in SEQ ID NOs:2 and 37, respectively.

Embodiment 37. The antibody or antigen-binding fragment of any one of Embodiments 27-36, which comprises an IgG1m3 allotype, an IgG1m17 allotype, an IgG1 ml allotype, or any combination thereof.

Embodiment 38. The antibody or antigen-binding fragment of any one of Embodiments 1-37, wherein the VH is comprised in a heavy chain that further comprises the CH1-CH3 amino acid sequence set forth in SEQ ID NO:34, SEQ ID NO:36, or SEQ ID NO:38, or comprises SEQ ID NO:34 with the C-terminal lysine and optionally the C-terminal glycine-lycine removed, SEQ ID NO:36 with the C-terminal glycine removed, or SEQ ID NO:38 with the C-terminal glycine removed.

Embodiment 39. The antibody or antigen-binding fragment of any one of Embodiments 1-38, wherein the VL is comprised in a light chain that further comprises the CL amino acid sequence set forth in SEQ ID NO:35.

Embodiment 40. An anti-influenza neuraminidase (anti-NA) antibody comprising (i) a heavy chain comprising or consisting of the amino acid sequence set forth in SEQ ID NO:39, or comprising or consisting of SEQ ID NO:39 with the C-terminal lysine removed or with the C-terminal glycine-lysine removed; and (ii) a light chain comprising or consisting of the amino acid sequence set forth in SEQ ID NO.41 Embodiment 41. An anti-influenza neuraminidase (anti-NA) antibody comprising (i) a heavy chain comprising or consisting of the amino acid sequence set forth in SEQ ID NO:40, or comprising or consisting of SEQ ID NO:40 with the C-terminal lysine removed or with the C-terminal glycine-lysine removed; and (ii) a light chain comprising or consisting of the amino acid sequence set forth in SEQ ID NO.41 Embodiment 42. An anti-influenza neuraminidase (anti-NA) antibody comprising (i) a heavy chain comprising or consisting of the amino acid sequence set forth in SEQ ID NO:42, or comprising or consisting of SEQ ID NO:42 with the C-terminal lysine removed, or with the C-terminal glycine-lysine removed; and (ii) a light chain comprising or consisting of the amino acid sequence set forth in SEQ ID NO:44.

Embodiment 43. An anti-influenza neuraminidase (anti-NA) antibody comprising (i) a heavy chain comprising or consisting of the amino acid sequence set forth in SEQ ID NO:43, or comprising or consisting of SEQ ID NO:43 with the C-terminal lysine removed, or with the C-terminal glycine-lysine removed; and (ii) a light chain comprising or consisting of the amino acid sequence set forth in SEQ ID NO:44.

Embodiment 44. An anti-influenza neuraminidase (anti-NA) antibody comprising (i) two heavy chains, each comprising or consisting of the amino acid sequence set forth in SEQ ID NO:39, or comprising or consisting of SEQ ID NO:39 with the C-terminal lysine removed, or with the C-terminal glycine-lysine removed; and (ii) two light chains, each comprising or consisting of the amino acid sequence set forth in SEQ ID NO.41 Embodiment 45. An anti-influenza neuraminidase (anti-NA) antibody comprising (i) two heavy chains, each comprising or consisting of the amino acid sequence set forth in SEQ ID NO:40, or with the C-terminal glycine-lysine removed, or comprising or consisting of SEQ ID NO:40 with the C-terminal lysine removed, or with the C-terminal glycine-lysine removed; and (ii) two light chains, each comprising or consisting of the amino acid sequence set forth in SEQ ID NO:41.

Embodiment 46. An anti-influenza neuraminidase (anti-NA) antibody comprising (i) two heavy chains, each comprising or consisting of the amino acid sequence set forth in SEQ ID NO:42, or comprising or consisting of SEQ ID NO:42 with the C-terminal lysine removed, or with the C-terminal glycine-lysine removed; and (ii) two light chains, each chain comprising or consisting of the amino acid sequence set forth in SEQ ID NO:44.

Embodiment 47. An anti-influenza neuraminidase (anti-NA) antibody comprising (i) two heavy chains, each comprising or consisting of the amino acid sequence set forth in SEQ ID NO:43, or comprising or consisting of SEQ ID NO:43 with the C-terminal lysine removed, or with the C-terminal glycine-lysine removed; and (ii) two light chains, each comprising or consisting of the amino acid sequence set forth in SEQ ID NO:44.

Embodiment 48. An anti-influenza neuraminidase (anti-NA) antibody or antigen-binding fragment comprising the VH amino acid sequence set forth in SEQ ID NO:54 and the VL amino acid sequence set forth in SEQ ID NO:8.

Embodiment 48a. An anti-influenza neuraminidase (anti-NA) antibody or antigen-binding fragment comprising (i) a VH comprising or consisting of the amino acid sequence of any one of SEQ ID NOs:2, 45, 46, 48, 50, 52, 54, 56, 58, 60, 63, and 65, and (ii) a VL comprising or consisting of the VL amino acid sequence of SEQ ID NO:8.

Embodiment 48b. An anti-influenza neuraminidase (anti-NA) antibody or antigen-binding fragment comprising: (i) a VH comprising the amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSS (SEQ ID NO:54); and (ii) a VL comprising the amino acid sequence

(SEQ ID NO.: 8)
EIVMTQSPATLSLSSGERATLSCRASRSVSSNLAWYQQKPGQAPRL
LIYDASTRATGFSARFAGSGSGTEFTLTISSLQSEDSAIYYCQQYN
NWPPWTFGQGTKVEIK.

Embodiment 48c. An anti-influenza neuraminidase (anti-NA) antibody or antigen-binding fragment comprising: (i) a VH consisting of the amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSS (SEQ ID NO:54); and (ii) a VL consisting of the amino acid sequence

(SEQ ID NO.: 8)
EIVMTQSPATLSLSSGERATLSCRASRSVSSNLAWYQQKPGQAPRL
LIYDASTRATGFSARFAGSGSGTEFTLTISSLQSEDSAIYYCQQYN
NWPPWTFGQGTKVEIK.

Embodiment 48d. An anti-influenza neuraminidase (anti-NA) antibody or antigen-binding fragment comprising: (i) a heavy chain comprising the VH amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSS (SEQ ID NO:54); and (ii) a light chain comprising the VL amino acid sequence

(SEQ ID NO.: 8)
EIVMTQSPATLSLSSGERATLSCRASRSVSSNLAWYQQKPGQAPRL
LIYDASTRATGFSARFAGSGSGTEFTLTISSLQSEDSAIYYCQQYN
NWPPWTFGQGTKVEIK,

wherein, optionally, the antibody or antigen-binding fragment is an (e.g. human) IgG1 isotype and optionally comprises M428L and N434S mutations, and/or wherein the light chain is an IgG1 kappa light chain.

Embodiment 48e. An anti-influenza neuraminidase (anti-NA) antibody or antigen-binding fragment comprising: (i) two heavy chains each comprising the VH amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSS (SEQ ID NO:54); and (ii) two light chains each comprising the VL amino acid sequence

(SEQ ID NO.: 8)
EIVMTQSPATLSLSSGERATLSCRASRSVSSNLAWYQQKPGQAPRL
LIYDASTRATGFSARFAGSGSGTEFTLTISSLQSEDSAIYYCQQYN
NWPPWTFGQGTKVEIK,

wherein, optionally, the antibody or antigen-binding fragment is an (e.g. human) IgG1 isotype and optionally comprises M428L and N434S mutations, and/or wherein the light chains are each an IgG1 kappa light chain.

Embodiment 48f. An anti-influenza neuraminidase (anti-NA) antibody or antigen-binding fragment comprising: (i) a VH comprising CDRH1, CDRH2, and CDRH3 of the VH amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSS (SEQ ID NO:54), wherein, optionally, the CDRs are defined according to IMGT; and (ii) a VL comprising CDRL1, CDRL2, and CDRL3 of the VL amino acid sequence

(SEQ ID NO.: 8)
EIVMTQSPATLSLSSGERATLSCRASRSVSSNLAWYQQKPGQAPRL
LIYDASTRATGFSARFAGSGSGTEFTLTISSLQSEDSAIYYCQQYN
NWPPWTFGQGTKVEIK,

wherein, optionally, the CDRs are defined according to IMGT.

Embodiment 48g. An anti-influenza neuraminidase (anti-NA) antibody or antigen-binding fragment comprising: (i) a heavy chain comprising, in a VH, CDRH1, CDRH2, and CDRH3 of the VH amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSS (SEQ ID NO:54), wherein, optionally, the CDRs are defined according to IMGT; and (ii) a light chain comprising, in a VL, CDRL1, CDRL2, and CDRL3 of the VL amino acid sequence

(SEQ ID NO.: 8)
EIVMTQSPATLSLSSGERATLSCRASRSVSSNLAWYQQKPGQAPRL
LIYDASTRATGFSARFAGSGSGTEFTLTISSLQSEDSAIYYCQQYN
NWPPWTFGQGTKVEIK,

wherein, optionally, the CDRs are defined according to IMGT, wherein, further optionally, the antibody or antigen-binding fragment is an (e.g. human) IgG1 isotype and optionally comprises M428L and N434S mutations, and/or wherein the light chain is an IgG1 kappa light chain.

Embodiment 48h. An anti-influenza neuraminidase (anti-NA) antibody or antigen-binding fragment comprising: (i) two heavy chains each comprising, in a VH, CDRH1, CDRH2, and CDRH3 of the VH amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSS (SEQ ID NO:54) wherein, optionally, the CDRs are defined according to IMGT; and (ii) two light chains each comprising, in a VL, CDRL1, CDRL2, and CDRL3 of the VL amino acid sequence

(SEQ ID NO.: 8)
EIVMTQSPATLSLSSGERATLSCRASRSVSSNLAWYQQKPGQAPRL
LIYDASTRATGFSARFAGSGSGTEFTLTISSLQSEDSAIYYCQQYN
NWPPWTFGQGTKVEIK,

wherein, optionally, the CDRs are defined according to IMGT, and wherein, further optionally, the antibody or antigen-binding fragment is an (e.g. human) IgG1 isotype and optionally comprises M428L and N434S mutations, and/or wherein the light chains are each an IgG1 kappa light chain.

Embodiment 49. An anti-influenza neuraminidase (anti-NA) antibody comprising (i) two heavy chains, wherein each of the two heavy chains comprises the VH amino acid sequence set forth in SEQ ID NO:54 and (ii) two light chains, wherein each of the two light chains comprises amino acid sequence set forth in SEQ ID NO:8, wherein, optionally, the antibody is an IgG1 isotype and wherein, further optionally, the antibody comprises M428L and N434S mutations (EU numbering).

Embodiment 50. The antibody or antigen-binding fragment of any one of Embodiments 1-49, comprising in a heavy chain thereof, the amino acid mutation(s) set forth in any one of (i)-(xviii): (i) G236A, L328V, and Q295E; (ii) G236A, P230A, and Q295E; (iii) G236A, R292P, and I377N; (iv) G236A, K334A, and Q295E; (v) G236S, R292P, and Y300L; (vi) G236A and Y300L; (vii) G236A, R292P, and Y300L; (viii) G236S, G420V, G446E, and L309T; (ix) G236A and R292P; (x) R292P and Y300L; (xi) G236A and R292P; (xii) Y300L; (xiii) E345K, G236S, L235Y, and S267E; (xiv) E272R, L309T, S219Y, and S267E; (xv) G236Y; (xvi) G236W; (xvii) F243L, G446E, P396L, and S267E; (xviii) G236A, S239D, and H268E.

Embodiment 51. The antibody or antigen-binding fragment of any one of Embodiments 1-50, comprising in a heavy chain thereof, the amino acid mutation G236A.

Embodiment 52. The antibody or antigen-binding fragment of any one of Embodiments 1-51, which: is afucosylated; has been produced in a host cell that is incapable of fucosylation or that is inhibited in its ability to fucosylate a polypeptide; has been produced under conditions that inhibit fucosylation thereof by a host cell; or any combination thereof.

Embodiment 53. The antibody or antigen-binding fragment of any one of Embodiments 1-52, which is human, humanized, or chimeric.

Embodiment 54. The antibody or antigen-binding fragment of any one of Embodiments 1-53, which is capable of binding to a NA from: (i) an influenza A virus (IAV), wherein the IAV comprises a Group 1 IAV, a Group 2 IAV, or both; and (ii) an influenza B virus (IBV).

Embodiment 55. The antibody or antigen-binding fragment of Embodiment 54, wherein: (i) the Group 1 IAV NA comprises a N1, a N4, a N5, and/or a N8; and/or (ii) the Group 2 IAV NA comprises a N2, a N3, a N6, a N7, and/or a N9.

Embodiment 56. The antibody or antigen-binding fragment of Embodiment 55, wherein: (i) the N1 is a N1 from any one or more of: A/California/07/2009, A/California/07/2009 I223R/H275Y, A/California/07/2009 Q250S, A/Swine/Jiangsu/J004/2018, A/Swine/Hebei/2017, A/Stockholm/18/2007, A/Brisbane/02/2018, A/Michigan/45/2015, A/Mississippi/3/2001, A/Netherlands/603/2009, A/Netherlands/602/2009, A/Vietnam/1203/2004, A/Vietnam/1203/2004 S247R, A/Vietnam/1203/2004 I223R, A/Vietnam/1203/2004 R152I, A/Vietnam/1203/2004 D199N, A/G4/SW/Shangdong/1207/2016, A/G4/SW/Henan/SN13/2018, A/G4/SW/Jiangsu/J004/2018, A/Mink/Spain/2022, and A/New Jersey/8/1976; (ii) the N4 is from A/mallard duck/Netherlands/30/2011; (iii) the N5 is from A/aquatic bird/Korea/CN5/2009; (iv) the N8 is from A/harbor seal/New Hampshire/179629/2011 or A/chicken/Russia/3-29/2020; (v) the N2 is a N2 from any one or more of: A/Washington/01/2007, A/Washington/01/2007 R292K, A/HongKong/68, A/South Australia/34/2019, A/Switzerland/8060/2017, A/Singapore/INFIMH-16-0019/2016, A/Switzerland/9715293/2013, A/Leningrad/134/17/57, A/Florida/4/2006, A/Netherlands/823/1992, A/Norway/466/2014, A/Switzerland/8060/2017, A/Texas/50/2012, A/Victoria/361/2011, A/HongKong/2671/2019, A/HongKong/2671/2019 K431E, A/SW/Mexico/SG1444/2011, A/Tanzania/205/2010, A/Aichi/2/1968, A/Bilthoven/21793/1972, A/Netherlands/233/1982, A/Shanghai/11/1987, A/Nanchang/933/1995, A/Fukui/45/2004, A/Brisbane/10/2007, A/Tasmania/503/2020 A/Cambodia/2020, A/Perth/16/2009, A/Kansas/14/2017, A/Swine/Kansas/2021, A/Canine/Korea/VC378/2012, and A/Canine/Indiana/003018/2016; (vi) the N3 is a N3 from any one or more of A/Canada/rv504/2004 and A/Ck/Ja/2017; (v) the N6 is a N6 from any one or more of A/swine/Ontario/01911/1/99, A/Ck/Suzhou/2019, and A/Hangzhou/2021; (vi) the N7 is a N7 from any one or more of A/Netherlands/078/03, A/Ck/621572/03; (vii) the N8 is from any one or more of A/harbor seal/New Hampshire/179629/2011 and A/Ck/Russia/2020; and/or (viii) the N9 is a N9 from any one or more of: A/Anhui/2013 and A/Hong Kong/56/2015.

Embodiment 57. The antibody or antigen-binding fragment of any one of Embodiments 54-56, wherein the IBV NA is a NA from any one or more of: B/Lee/10/1940 (Ancestral); B/Brisbane/60/2008 (Victoria); B/Malaysia/2506/2004 (Victoria); B/Malaysia/3120318925/2013 (Yamagata); B/Wisconsin/1/2010 (Yamagata); B/Yamanashi/166/1998 (Yamagata); B/Brisbane/33/2008; B/Colorado/06/2017; B/Hubei-wujiang/158/2009; B/Massachusetts/02/2012; B/Netherlands/234/2011; B/Perth/211/2001; B/Texas/06/2011 (Yamagata); B/Perth/211/2011; B/HongKong/05/1972; B/Phuket/3073/2013, B/Harbin/7/1994 (Victoria), B/Washington/02/2019 (Victoria); B/Victoria/504/2000 (Yamagata); B/Victoria/2/87; B/Victoria/2/87-lineage; B/Yamagata/16/88; and B/Yamagata/16/88-lineage.

Embodiment 58. The antibody or antigen-binding fragment of any one of Embodiments 1-57, wherein the antibody or antigen-binding fragment is capable of binding to each of: (i) a Group 1 IAV NA; (ii) a Group 2 IAV NA; and (iii) a IBV NA with an EC₅₀ in a range from about 0.1 μg/mL to about 50 μg/mL, or in a range from about 0.1 μg/mL to about 2 μg/mL, or in a range from 0.1 μg/mL to about 10 μg/mL, or in a range from 2 μg/mL to about 10 μg/mL, or in a range from about 0.4 μg/mL to about 50 μg/mL, or in a range from about 0.4 μg/mL to about 2 μg/mL, or in a range from 0.4 μg/mL to about 10 μg/mL, or in a range from 2 μg/mL to about 10 μg/mL, or in a range from 0.4 μg/mL to about 1 μg/mL, or 0.4 μg/mL or less.

Embodiment 59. The antibody or antigen-binding fragment of Embodiment 58, wherein the antibody or antigen-binding fragment is capable of binding to: (i) the Group 1 IAV NA with an EC₅₀ in a range from about 0.4 μg/mL to about 50 μg/mL, from about 0.4 μg/mL to about 10 μg/mL, from about 0.4 μg/mL to about 2 μg/mL, from about 2 μg/mL to about 50 μg/mL, from about 2 μg/mL to about 10 μg/mL, or from about 10 μg/mL to about 50 μg/mL; (ii) the Group 2 IAV NA with an EC₅₀ in a range from about 0.4 μg/mL to about 50 μg/mL, or from about 0.4 μg/mL to about 10 μg/mL, or from about 0.4 μg/mL to about 2 μg/mL, or from about 2 μg/mL to about 50 μg/mL, or from about 2 μg/mL to about 10 μg/mL, or from about 10 μg/mL to about 50 μg/mL; and/or (iii) the IBV NA with an EC₅₀ of about 0.4 μg/mL, or in a range from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL.

Embodiment 60. The antibody or antigen-binding fragment of Embodiment 59, wherein the antibody or antigen-binding fragment is capable of binding to: (i) a N1 with an EC₅₀ of about 0.4 μg/mL, or in a range from about 0.4 μg/mL to about 50 μg/mL, or in a range from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL; (ii) a N4 with an EC₅₀ of about 0.4 μg/mL, or in a range from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL; (iii) a N5 with an EC₅₀ in a range from about 0.4 μg/mL to about 2 μg/mL; (iv) a N8 with an EC₅₀ of about 50 μg/mL; (v) a N2 with an EC₅₀ in a range from about 0.4 μg/mL to about 20 μg/mL, or from about 0.4 μg/mL to about 10 μg/mL, or from about 0.4 μg/mL to about 2 μg/mL, from about 1 μg/mL to about 10 μg/mL, or from about 1 μg/mL to about 20 μg/mL, or from about 1 μg/mL to about 5 μg/mL, or about 1, 2, 3, 4, 5, 6, 7, 8, 9, 10, 15, or 20 μg/mL; (vi) a N3 with an EC₅₀ of about 0.4 μg/mL, or in a range from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL; (vii) a N6 with an EC₅₀ of about 0.4 μg/mL, or in a range from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL; (viii) a N7 with an EC₅₀ in a range from about 2 μg/mL to about 50 μg/mL; (ix) a N9 with an EC₅₀ of about 0.4 μg/mL, or in a range from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL; and/or (xi) a IBV NA with an EC₅₀ of about 0.4 μg/mL, or in a range from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL.

Embodiment 61. The antibody or antigen-binding fragment of Embodiment 59 or 60, wherein the antibody or antigen-binding fragment is capable of binding to: (i) one or more of: N1 A/California/07/2009, N1 A/California/07/2009 I223R/H275Y, N1 A/Stockholm/18/2007, N1 A/Swine/Jiangsu/J004/2008, N4 A/mallard duck/Netherlands/30/2011, N5 A/aquatic bird/Korea/CN5/2009, N2 A/Hong Kong/68, N2 A/Leningrad/134/17/57, N3 A/Canada/rv504/2004, N6 A/Swine/Ontario/01911/1/99, N9 A/Anhui/1/2013, B/Lee/10/1940 (Ancestral), B/Brisbane/60/2008 (Victoria), B/Malaysia/2506/2004 (Victoria), B/Malaysia/3120318925/2013 (Yamagata), B/Wisconsin/l/2010 (Yamagata), and B/Yamanashi/166/1998 (Yamagata), with an EC₅₀ of about 0.4 μg/mL, or in a range of from about 0.1 μg/mL to about 1.9 μg/mL, or of from about 0.1 μg/mL to about 1.5 μg/mL, or of from about 0.1 μg/mL to about 1.0 μg/mL, or about 0.1, 0.2, 0.3, 0.4, 0.5, 0.6, 0.7, 0.8, 0.9, or 1.0 μg/mL; (ii) N5 A/aquatic bird/Korea/CN5/2009 with an EC₅₀ of about 2 μg/mL, or in a range of from about 2 μg/mL to about 10 μg/mL; (iii) N8 A/harbor seal/New Hampshire/179629/2011 with an EC₅₀ of about 50 μg/mL; (iv) N2 A/Washington/01/2007 with an EC₅₀ in a range from about 2 μg/mL to about 10 μg/mL; (v) N7 A/Netherlands/078/03 with an EC₅₀ in a range from about 2 μg/mL to about 50 μg/mL; (vi) N2 A/South Australia/34/2019 with an EC₅₀ in a range of from about 0.4 μg/mL to about 50 μg/mL; (vii) N2 A/Switzerland/8060/2017 with an EC₅₀ in a range of from about 9.5 μg/mL to about 3.8 μg/mL; (viii) N2 A/Singapore/INFIMH-16-0019/2016 with an EC₅₀ in a range of from about 18.4 μg/mL to about 2.2 μg/mL; (iv) N2 A/Switzerland/9715293/2013 with an EC₅₀ in a range of from about 1.6 μg/mL to about 1.2 μg/mL; and/or (v) N1 A/Swine/Jiangsu/J004/2018 with an EC₅₀ in a range of from about 0.4 μg/mL to about 50 μg/mL, or about 0.4, about 2, about 10, or about 50 μg/mL.

Embodiment 62. The antibody or antigen-binding fragment of any one of Embodiments 1-61, wherein the NA is expressed on the surface of a host cell (e.g., a CHO cell) and binding to NA is according to flow cytometry.

Embodiment 63. The antibody or antigen-binding fragment of any one of Embodiments 1-62, which is capable of binding to a NA with a KD of less than 1.0E-12 M, less than 1.0E-11 M, less than 1.0 E-11 M, or of 1.0E-12M or less, 1.0E-11M or less, or 1.0E-10 or less, or with a KD between 1.0E-10 and 1.0E-13, or with a KD between 1.0E-11 and 1.0E-13, wherein, optionally, the binding is as assessed by biolayer interferometry (BLI).

Embodiment 64. The antibody or antigen-binding fragment of Embodiment 63, wherein the NA is a N1, a N2, and/or a N9.

Embodiment 65. The antibody or antigen-binding fragment of any one of Embodiments 1-64, which is capable of binding to: (1) (i) a NA epitope that comprises any one or more of the following amino acids (N1 NA numbering): R368, R293, E228, E344, S247, D198, D151, R118; and/or (ii) a NA epitope that comprises any one or more of the following amino acids (N2 NA numbering): R371, R292, E227, E344, S247, D198, D151, R118; and/or (2) (i) a NA epitope that comprises the amino acids R368, R293, E228, D151, and R118 (N1 NA numbering); and/or (ii) a NA epitope that comprises the amino acids R371, R292, E227, D151, and R118 (N2 NA numbering); and/or (3) an epitope comprised in or comprising a NA active site, wherein, optionally, the NA active site comprises the following amino acids (N2 numbering): R118, D151, R152, R224, E276, R292, R371, Y406, E119, R156, W178, S179, D/N198, 1222, E227, H274, E277, D293, E425; and/or (4) an IBV NA epitope that comprises: (i) any one or more of the following amino acids: R116, D149, E226, R292, and R374; or (ii) the amino acids R116, D149, E226, R292, and R374.

Embodiment 66. The antibody or antigen-binding fragment of Embodiment 65, wherein: (1) the epitope further comprises any one or more of the following NA amino acids (N2 numbering): E344, E227, S247, and D198; and/or (2) the antibody or antigen-binding fragment is capable of binding to a NA comprising a S245N amino acid mutation and/or a E221D amino acid mutation.

Embodiment 67. The antibody or antigen-binding fragment of any one of Embodiments 1-66, which is capable of binding to a NA comprising a S245N amino acid mutation and/or a E221D amino acid mutation.

Embodiment 68. The antibody or antigen-binding fragment of any one of Embodiments 1-67, wherein the antibody or antigen-binding fragment is capable of inhibiting a sialidase activity of (i) an IAV NA, wherein the IAV NA comprises a Group 1 IAV NA, a Group 2 IAV NA, or both, and/or of (ii) an IBV NA in an in vitro model of infection, an in vivo animal model of infection, and/or in a human.

Embodiment 69. The antibody or antigen-binding fragment of Embodiment 68, wherein: (i) the Group 1 IAV NA comprises a H1N1 and/or a H5N1; (ii) the Group 2 IAV NA comprises a H3N2 and/or a H7N9; and/or (iii) the IBV NA comprises one or more of: B/Lee/10/1940 (Ancestral); B/HongKong/05/1972; B/Taiwan/2/1962 (Ancestral); B/Brisbane/33/2008 (Victoria); B/Brisbane/60/2008 (Victoria); B/Malaysia/2506/2004 (Victoria); B/New York/1056/2003 (Victoria); B/Florida/4/2006 (Yamagata); B/Jiangsu/10/2003 (Yamagata); B/Texas/06/2011 (Yamagata); B/Perth/211/2011; B/Harbin/7/1994 (Victoria); B/Colorado/06/2017 (Victoria); B/Washington/02/2019 (Victoria); B/Perth/211/2001 (Yamagata); B/Hubei-wujiagang/158/2009 (Yamagata); B/Wisconsin/01/2010 (Yamagata); B/Massachusetts/02/2012 (Yamagata); and B/Phuket/3073/2013 (Yamagata).

Embodiment 70. The antibody or antigen-binding fragment of any one of Embodiments 1-69, wherein the antibody or antigen-binding fragment is capable of inhibiting a sialidase activity by: a Group 1 IAV NA; a Group 2 IAV NA; and/or a IBV NA, with an IC50 in a range of from about 0.0008 μg/mL to about 4 μg/mL, from about 0.0008 μg/mL to about 3 μg/mL, from about 0.0008 μg/mL to about 2 μg/mL, from about 0.0008 μg/mL to about 1 μg/mL, from about 0.0008 μg/mL to about 0.9 μg/mL, from about 0.0008 μg/mL to about 0.8 μg/mL, from about 0.0008 μg/mL to about 0.7 μg/mL, from about 0.0008 μg/mL to about 0.6 μg/mL, from about 0.0008 μg/mL to about 0.5 μg/mL, from about 0.0008 μg/mL to about 0.4 μg/mL, from about 0.0008 μg/mL to about 0.3 μg/mL, from about 0.0008 μg/mL to about 0.2 μg/mL, from about 0.0008 μg/mL to about 0.1 μg/mL, from about 0.0008 μg/mL to about 0.09 μg/mL, from about 0.0008 μg/mL to about 0.08 μg/mL, from about 0.0008 μg/mL to about 0.07 μg/mL, from about 0.0008 μg/mL to about 0.06 μg/mL, about 0.0008 μg/mL to about 0.05 μg/mL, about 0.0008 μg/mL to about 0.04 μg/mL, about 0.0008 μg/mL to about 0.03 μg/mL, about 0.0008 μg/mL to about 0.02 μg/mL, about 0.0008 μg/mL to about 0.01 μg/mL, from 0.002 μg/mL to about 4 μg/mL, from about 0.001 μg/mL to 50 μg/mL, from about 0.1 μg/mL to about 30 μg/mL, from about 0.1 μg/mL to about 20 μg/mL, from about 0.1 μg/mL to about 10 μg/mL, from about 0.1 μg/mL to about 9 μg/mL, from about 0.1 μg/mL to about 8 μg/mL, from about 0.1 μg/mL to about 7 μg/mL, from about 0.1 μg/mL to about 6 μg/mL, from about 0.1 μg/mL to about 5 μg/mL, from about 0.1 μg/mL to about 4 μg/mL, from about 0.1 μg/mL to about 3 μg/mL, from about 0.1 μg/mL to about 2 μg/mL, from about 0.1 μg/mL to about 1 μg/mL, from about 0.1 μg/mL to about 0.9 μg/mL, from about 0.1 μg/mL to about 0.8 μg/mL, from about 0.1 μg/mL to about 0.7 μg/mL, from about 0.1 μg/mL to about 0.6 μg/mL, from about 0.1 μg/mL to about 0.5 μg/mL, from about 0.1 μg/mL to about 0.4 μg/mL, from about 0.1 μg/mL to about 0.3 μg/mL, from about 0.1 μg/mL to about 0.2 μg/mL, from about 0.8 μg/mL to about 30 μg/mL, from about 0.8 μg/mL to about 20 μg/mL, from about 0.8 μg/mL to about 10 μg/mL, from about 0.8 μg/mL to about 9 μg/mL, from about 0.8 μg/mL to about 8 μg/mL, from about 0.8 μg/mL to about 7 μg/mL, from about 0.8 μg/mL to about 6 μg/mL, from about 0.8 μg/mL to about 5 μg/mL, from about 0.8 μg/mL to about 4 μg/mL, from about 0.8 μg/mL to about 3 μg/mL, from about 0.8 μg/mL to about 2 μg/mL, from about 0.8 μg/mL to about 1 μg/mL, or of about 0.1 μg/mL, about 0.2 μg/mL, about 0.3 μg/mL, about 0.4 μg/mL, about 0.5 μg/mL, about 0.6 μg/mL, about 0.7 μg/mL, about 0.8 μg/mL, about 0.9 μg/mL, about 1.0 μg/mL, about 1.5 μg/mL, about 2.0 μg/mL, about 2.5 μg/mL, about 3.0 μg/mL, about 3.5 μg/mL, about 4.0 μg/mL, about 4.5 μg/mL, about 5.0 μg/mL, about 5.5 μg/mL, about 6.0 μg/mL, about 6.5 μg/mL, about 7.0 μg/mL, about 7.5 μg/mL, about 8.0 μg/mL, about 8.5 μg/mL, about 9.0 μg/mL, about 10 μg/mL, about 11 μg/mL, about 12 μg/mL, about 13 μg/mL, about 14 μg/mL, about 15 μg/mL, about 16 μg/mL, about 17 μg/mL, about 18 μg/mL, about 19 μg/mL, about 20 μg/mL, about 25 μg/mL, and/or about 30 μg/mL.

Embodiment 71. The antibody or antigen-binding fragment of Embodiment 70, which is capable of inhibiting NA sialidase activity of one or more Group 1 and/or Group 2 IAV, and/or of one or more IBV, with an IC50 in a range of from: about 0.00001 μg/ml to about 25 μg/ml, or about 0.0001 μg/ml to about 10 μg/ml, or about 0.0001 μg/ml to about 1 μg/ml, or about 0.0001 μg/ml to about 0.1 μg/ml, or about 0.0001 μg/ml to about 0.01 μg/ml, or about 0.0001 μg/ml to about 0.001 μg/ml, or about 0.0001 μg/ml to about 0.0001 μg/ml, or about 0.0001 μg/ml to about 25 μg/ml, or about 0.0001 μg/ml to about 10 μg/ml, or about 0.0001 μg/ml to about 1 μg/ml, or about 0.0001 μg/ml to about 0.1 μg/ml, or about 0.0001 μg/ml to about 0.01 μg/ml, or about 0.001 μg/ml to about 25 μg/ml, or about 0.001 μg/ml to about 10 μg/ml, or about 0.001 μg/ml to about 1 μg/ml, or about 0.001 μg/ml to about 0.1 μg/ml, or about 0.001 μg/ml to about 0.01 μg/ml, or about 0.01 μg/ml to about 25 μg/ml, or about 0.01 μg/ml to about 10 μg/ml, or about 0.01 μg/ml to about 1 μg/ml, or about 0.01 μg/ml to about 0.1 μg/ml, or about 1 μg/ml to about 25 μg/ml, or about 1 μg/ml to about 10 μg/ml, or of about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 μg/ml.

Embodiment 72. The antibody or antigen-binding fragment of any one of Embodiments 1-71, which is capable of activating a human FcγRIIIa.

Embodiment 73. The antibody or antigen-binding fragment of Embodiment 72, wherein activation is as determined using a host cell (optionally, a Jurkat cell) comprising: (i) the human FcγRIIIa (optionally, a F158 allele); and (ii) a NFAT expression control sequence operably linked to a sequence encoding a reporter, such as a luciferase reporter, following incubation (e.g., of 23 hours) of the antibody or antigen-binding fragment with a target cell (e.g., a A549 cell) infected with a IAV.

Embodiment 74. The antibody or antigen-binding fragment of Embodiment 73, wherein activation is as determined following an incubation (optionally, for about 23 hours) of the antibody or antigen-binding fragment with the target cell infected with a H1N1 IAV, wherein, optionally, the H1N1 IAV is A/PR8/34, and/or wherein, optionally, the infection has a multiplicity of infection (MOI) of 6.

Embodiment 75. The antibody or antigen-binding fragment of any one of Embodiments 1-74, which is capable of neutralizing infection by an IAV and/or an IBV.

Embodiment 76. The antibody or antigen-binding fragment of Embodiment 75, wherein the IAV and/or the IBV is antiviral-resistant, wherein, optionally, the antiviral is oseltamivir.

Embodiment 77. The antibody or antigen-binding fragment of any one of Embodiments 54-76, wherein the IAV comprises a N1 NA that comprises the amino acid mutation(s): H275Y; E119D+H275Y; S247N+H275Y; I222V; and/or N294S, wherein, optionally, the IAV comprises CA09 or A/Aichi.

Embodiment 78. The antibody or antigen-binding fragment of any one of Embodiments 54-77, wherein the IAV comprises a N2 NA that comprises the amino acid mutation(s) E119V, Q136K, and/or R292K.

Embodiment 79. The antibody or antigen-binding fragment of any one of Embodiments 1-78, wherein the antibody or antigen-binding fragment is capable of treating and/or preventing (i) an IAV infection and/or (ii) an IBV infection, in a subject.

Embodiment 80. The antibody or antigen-binding fragment of any one of Embodiments 1-79, wherein the antibody or antigen-binding fragment is capable of treating and/or attenuating an infection by: (i) a H1N1 virus, wherein, optionally, the H1N1 virus comprises A/PR8/34; and/or (ii) a H3N2 virus, wherein, optionally, the H3N2 virus optionally comprises A/Hong Kong/68.

Embodiment 81. The antibody or antigen-binding fragment of any one of Embodiments 1-80, wherein the antibody or antigen-binding fragment is capable of preventing weight loss in a subject infected by the IAV and/or IBV, optionally for (i) up to 15 days, or (ii) more than 15 days, following administration of an effective amount of the antibody or antigen-binding fragment.

Embodiment 82. The antibody or antigen-binding fragment of any one of Embodiments 1-81, wherein the antibody or antigen-binding fragment is capable of preventing a loss in body weight of greater than 10% in a subject having an IAV infection and/or an IBV infection, as determined by reference to the subject's body weight just prior to the IAV and/or IBV infection.

Embodiment 83. The antibody or antigen-binding fragment of any one of Embodiments 1-82, wherein the antibody or antigen-binding fragment is capable extending survival of a subject having an IAV infection and/or an IBV infection.

Embodiment 84. The antibody or antigen-binding fragment of any one of Embodiments 1-83, wherein the antibody or antigen-binding fragment has an in vivo half-life in a mouse (e.g., a tg32 mouse): (i) in a range of from: about 10 days to about 14 days, about 10.2 days to about 13.8 days, about 10.5 days to about 13.5 days, about 11 days to about 13 days, about 11.5 days to about 12.5 days, between 10 days and 14 days, or between 10.5 days and 13.5 days, or between 11 days and 13 days, or of about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, or 14.0 days; or (ii) in a range of from about 12 days to about 16 days, about 12.5 days to 15.5 days, about 13 days to 15 days, about 13.5 days to about 14.5 days, or between 12 days and 16 days, or between 13 days and 15 days, or between 13.5 days and 14.5 days, or of about 12.0, 12.1, 12.2, 12.3, 12.4, 12.5, 12.6, 12.7, 12.8, 12.9, 13.0, 13.1, 13.2, 13.3, 13.4, 13.5, 1.36, 13.7, 13.8, 13.9, 14.0, 14.1, 14.2, 14.3, 14.4, 14.5, 14.6, 14.7, 14.8, 14.9, 15.0 15.1, 15.2, 15.3, 15.4, 15.5, 1.56, 15.7, 15.8, 15.9, or 16.0 days.

Embodiment 85. The antibody or antigen-binding fragment of Embodiment 83 or 84, wherein the antibody or antigen-binding fragment is capable of binding to a neuraminidase (NA) from: (i) an influenza A virus (IAV), wherein the IAV comprises a Group 1 IAV, a Group 2 IAV, or both; and/or (ii) an influenza B virus (IBV), and wherein, optionally, the antibody or antigen-binding fragment is capable of (1) inhibiting NA sialidase activity and/or (2) neutralizing infection by the IAV and/or IBV.

Embodiment 85a. An antibody comprising:
(i) a heavy chain comprising the amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSSASTKGPSVFPLAPSSKSTSGGTAALGCLVKDYFPEPVTVSWNSGALTS GVHTFPAVLQSSGLYSLSSVVTVPSSSLGTQTYICNVNHKPSNTKVDKRVEPKSCDKTHT CPPCPAPELLGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKAKGQ PREPQVYTLPPSREEMTKNQVSLTCLVKGFYPSDIAVEWESNGQPENNYKTTPPVLDSD GSFFLYSKLTVDKSRWQQGNVFSCSVLHEALHSHYTQKSLSLSPGK (SEQ ID NO:107), or SEQ ID NO:107 with the C-terminal lysine removed or the C-terminal glycine-lysine removed; and
(ii) a light chain comprising the amino acid sequence

(SEQ ID NO.: 108)
EIVMTQSPATLSLSSGERATLSCRASRSVSSNLAWYQQKPGQAPRL
LIYDASTRATGFSARFAGSGSGTEFTLTISSLQSEDSAIYYCQQYN
NWPPWTFGQGTKVEIKRTVAAPSVFIFPPSDEQLKSGTASVVCLLN
NFYPREAKVQWKVDNALQSGNSQESVTEQDSKDSTYSLSSTLTLSK
ADYEKHKVYACEVTHQGLSSPVTKSFNRGEC.

Embodiment 85b. An antibody comprising:
(i) a heavy chain consisting of the amino acid sequence

(SEQ ID NO.: 107)
QVHLVQSGAEVKEPGSSVTVSCKASGGTFNNQAISWVRQAPGQGLE
WMGGIFPISGTPTSAQRFQGRVTFTADESTTTVYMDLSSLRSDDTA
VYYCARAGSDYFNRDLGWENYYFASWGQGTLVTVSSASTKGPSVFP
LAPSSKSTSGGTAALGCLVKDYFPEPVTVSWNSGALTSGVHTFPAV
LQSSGLYSLSSVVTVPSSSLGTQTYICNVNHKPSNTKVDKRVEPKS
CDKTHTCPPCPAPELLGGPSVFLFPPKPKDTLMISRTPEVTCVVVD
VSHEDPEVKFNWYVDGVEVHNAKTKPREEQYNSTYRVVSVLTVLHQ
DWLNGKEYKCKVSNKALPAPIEKTISKAKGQPREPQVYTLPPSREE
MTKNQVSLTCLVKGFYPSDIAVEWESNGQPENNYKTTPPVLDSDGS
FFLYSKLTVDKSRWQQGNVFSCSVLHEALHSHYTQKSLSLSPGK,

or SEQ ID NO.: 107 with the C-terminal lysine removed or the C-terminal glycine-lysine removed; and
(ii) a light chain consisting of the amino acid sequence

(SEQ ID NO.: 108)
EIVMTQSPATLSLSSGERATLSCRASRSVSSNLAWYQQKPGQAPRL
LIYDASTRATGFSARFAGSGSGTEFTLTISSLQSEDSAIYYCQQYN
NWPPWTFGQGTKVEIKRTVAAPSVFIFPPSDEQLKSGTASVVCLLN
NFYPREAKVQWKVDNALQSGNSQESVTEQDSKDSTYSLSSTLTLSK
ADYEKHKVYACEVTHQGLSSPVTKSFNRGEC.

Embodiment 85c. An antibody comprising:
(i) two heavy chains, wherein each of the two heavy chains comprises the amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSSASTKGPSVFPLAPSSKSTSGGTAALGCLVKDYFPEPVTVSWNSGALTS GVHTFPAVLQSSGLYSLSSVVTVPSSSLGTQTYICNVNHKPSNTKVDKRVEPKSCDKTHT CPPCPAPELLGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKAKGQ PREPQVYTLPPSREEMTKNQVSLTCLVKGFYPSDIAVEWESNGQPENNYKTTPPVLDSD GSFFLYSKLTVDKSRWQQGNVFSCSVLHEALHSHYTQKSLSLSPGK (SEQ ID NO.: 107), or SEQ ID NO:107 with the C-terminal lysine removed or the C-terminal glycine-lysine removed; and
(ii) two light chains, wherein each of the two light chains comprises the amino acid sequence

(SEQ ID NO.: 108)
EIVMTQSPATLSLSSGERATLSCRASRSVSSNLAWYQQKPGQAPRL
LIYDASTRATGFSARFAGSGSGTEFTLTISSLQSEDSAIYYCQQYN
NWPPWTFGQGTKVEIKRTVAAPSVFIFPPSDEQLKSGTASVVCLLN
NFYPREAKVQWKVDNALQSGNSQESVTEQDSKDSTYSLSSTLTLSK
ADYEKHKVYACEVTHQGLSSPVTKSFNRGEC.

Embodiment 85d. An antibody comprising:
(i) two heavy chains, wherein each of the two heavy chains consists of the amino acid sequence

(SEQ ID NO.: 107)
QVHLVQSGAEVKEPGSSVTVSCKASGGTFNNQAISWVRQAPGQGLE
WMGGIFPISGTPTSAQRFQGRVTFTADESTTTVYMDLSSLRSDDTA
VYYCARAGSDYFNRDLGWENYYFASWGQGTLVTVSSASTKGPSVFP
LAPSSKSTSGGTAALGCLVKDYFPEPVTVSWNSGALTSGVHTFPAV
LQSSGLYSLSSVVTVPSSSLGTQTYICNVNHKPSNTKVDKRVEPKS
CDKTHTCPPCPAPELLGGPSVFLFPPKPKDTLMISRTPEVTCVVVD
VSHEDPEVKFNWYVDGVEVHNAKTKPREEQYNSTYRVVSVLTVLHQ
DWLNGKEYKCKVSNKALPAPIEKTISKAKGQPREPQVYTLPPSREE
MTKNQVSLTCLVKGFYPSDIAVEWESNGQPENNYKTTPPVLDSDGS
FFLYSKLTVDKSRWQQGNVFSCSVLHEALHSHYTQKSLSLSPGK,

or SEQ ID NO:107 with the C-terminal lysine removed or the C-terminal glycine-lysine removed; and
(ii) two light chains, wherein each of the two light chains consists of the amino acid sequence

(SEQ ID NO.: 108)
EIVMTQSPATLSLSSGERATLSCRASRSVSSNLAWYQQKPGQAPRL
LIYDASTRATGFSARFAGSGSGTEFTLTISSLQSEDSAIYYCQQYN
NWPPWTFGQGTKVEIKRTVAAPSVFIFPPSDEQLKSGTASVVCLLN
NFYPREAKVQWKVDNALQSGNSQESVTEQDSKDSTYSLSSTLTLSK
ADYEKHKVYACEVTHQGLSSPVTKSFNRGEC.

Embodiment 86. An isolated polynucleotide encoding the antibody or antigen-binding fragment of any one of Embodiments 1-85d, or encoding a VH, a Fd, a heavy chain, a VL, and/or a light chain of the antibody or the antigen-binding fragment, wherein, optionally: (1) the heavy chain comprises or consists of SEQ ID NO:107 or SEQ ID NO:107 with the C-terminal lysine removed or the C-terminal glycine removed; (2) the light chain comprises or consists of SEQ ID NO:108; (3) the polynucleotide comprises SEQ ID NO:109; and/or (4) the polynucleotide comprises SEQ ID NO:110.

Embodiment 87. The polynucleotide of Embodiment 86, wherein the polynucleotide comprises deoxyribonucleic acid (DNA) or ribonucleic acid (RNA), wherein the RNA optionally comprises messenger RNA (mRNA).

Embodiment 88. The polynucleotide of any one of Embodiments 86-87, comprising a modified nucleoside, a cap-1 structure, a cap-2 structure, or any combination thereof.

Embodiment 89. The polynucleotide of Embodiment 88, wherein the polynucleotide comprises a pseudouridine, a N6-methyladenonsine, a 5-methylcytidine, a 2-thiouridine, or any combination thereof.

Embodiment 90. The polynucleotide of Embodiment 89, wherein the pseudouridine comprises N1-methylpseudouridine.

Embodiment 91. The polynucleotide of any one of Embodiments 86-90, which is codon-optimized for expression in a host cell.

Embodiment 92. The polynucleotide of Embodiment 91, wherein the host cell comprises a human cell.

Embodiment 93. A recombinant vector comprising the polynucleotide of any one of Embodiments 86-92, wherein, optionally, the polynucleotide encodes: (1) SEQ ID NO:107, or SEQ ID NO:107 with the C-terminal lysine removed or the C-terminal glycine-lysine removed; and (2) SEQ ID NO:108, wherein, further optionally, the vector comprises SEQ ID NO:109 and SEQ ID NO.: 110.

Embodiment 94. A host cell comprising the polynucleotide of any one of Embodiments 86-92 and/or the vector of Embodiment 93, wherein the polynucleotide is optionally heterologous to the host cell and/or wherein the host cell is capable of expressing the encoded antibody or antigen-binding fragment or polypeptide.

Embodiment 95. An isolated human B cell comprising the polynucleotide of any one of Embodiments 86-92 and/or the vector of Embodiment 93, wherein polynucleotide is optionally heterologous to the human B cell and/or wherein the human B cell is immortalized.

Embodiment 96. A composition comprising: (i) the antibody or antigen-binding fragment of any one of Embodiments 1-85d; (ii) the polynucleotide of any one of Embodiments 86-92; (iii) the recombinant vector of Embodiment 93; (iv) the host cell of Embodiment 94; and/or (v) the human B cell of Embodiment 95, and a pharmaceutically acceptable excipient, carrier, or diluent.

Embodiment 97. The composition of Embodiment 96, comprising a first antibody or antigen-binding fragment and a second antibody or antigen-binding fragment, wherein each of the first antibody or antigen-binding fragment and the second antibody or antigen-binding fragment are different and are each according to any one of Embodiments 1-85d.

Embodiment 97a. The composition of Embodiment 96, comprising the polynucleotide of any one of embodiments 86-92, wherein the polynucleotide encodes (i) SEQ ID NO:107 or SEQ ID NO:107 with the C-terminal lysine or C-terminal glycine-lysine removed, and (ii) SEQ ID NO:108, and the polynucleotide optionally comprises SEQ ID NO:109 and SEQ ID NO:110.

Embodiment 97b. The composition of Embodiment 96, comprising (i) a first polynucleotide encoding SEQ ID NO:107 or SEQ ID NO:107 with the C-terminal lysine or C-terminal glycine-lysine removed, wherein, optionally, the first polynucleotide comprises SEQ ID NO:109 and (ii) a second polynucleotide encoding SEQ ID NO:108, wherein, optionally, the second polynucleotide comprises SEQ ID NO.: 110.

Embodiment 97c. The composition of Embodiment 96, comprising (1) a first plasmid or vector comprising a polynucleotide encoding SEQ ID NO:107 or SEQ ID NO:107 with the C-terminal lysine or C-terminal glycine-lysine removed, wherein, optionally, the polynucleotide comprises SEQ ID NO.: 109, and (2) a second plasmid or vector comprising a polynucleotide encoding SEQ ID NO.: 108, wherein, optionally, the polynucleotide comprises SEQ ID NO:110.

Embodiment 98. A composition comprising the polynucleotide of any one of Embodiments 86-92 or the vector of Embodiment 93 encapsulated in a carrier molecule, wherein the carrier molecule optionally comprises a lipid, a lipid-derived delivery vehicle, such as a liposome, a solid lipid nanoparticle, an oily suspension, a submicron lipid emulsion, a lipid microbubble, an inverse lipid micelle, a cochlear liposome, a lipid microtubule, a lipid microcylinder, lipid nanoparticle (LNP), or a nanoscale platform.

Embodiment 99. The polynucleotide of any one of Embodiments 86-92, the vector of Embodiment 93, or the composition of Embodiment 96 or 98, comprising a first polynucleotide (e.g., mRNA) encoding an antibody heavy chain comprising the VH set forth in SEQ ID NO.: 54 and a second polynucleotide (e.g., mRNA) encoding an antibody light chain comprising the VL set forth in SEQ ID NO:8.

Embodiment 100. The polynucleotide of any one of Embodiments 86-92, the vector of Embodiment 93, or the composition of Embodiment 96 or 98, comprising a first polynucleotide (e.g., mRNA) encoding an antibody heavy chain comprising CDRH1, CDRH2, and CDRH3 sequences as set forth in SEQ ID NOs: (i) 55, 4, and 5, respectively, and a second polynucleotide (e.g., mRNA) encoding an antibody light chain comprising CDRL1, CDRL2, and CDRL3 sequences as set forth in SEQ ID Nos.: 9-11, respectively.

Embodiment 101. A method of making an antibody or antigen-binding fragment of any one of Embodiments 1-85d, comprising culturing the host cell of Embodiment 94 or the human B cell of Embodiment 95 for a time and under conditions sufficient for the host cell or human B cell, respectively, to express the antibody or antigen-binding fragment.

Embodiment 102. The method of Embodiment 101, further comprising isolating the antibody or antigen-binding fragment.

Embodiment 103. A method of treating or preventing an IAV infection and/or an IBV infection in a subject, the method comprising administering to the subject an effective amount of: (i) the antibody or antigen-binding fragment of any one of Embodiments 1-85d; (ii) the polynucleotide of any one of Embodiments 86-92, 99, and 100; (iii) the recombinant vector of Embodiment 93, 99, or 100; (iv) the host cell of Embodiment 94; (v) the human B cell of Embodiment 95; and/or (vi) the composition of any one of Embodiments 96-100.

Embodiment 104. The method according to Embodiment 103, wherein the antibody or antigen-binding fragment is administered to the subject at a dose of about 3 mg/kg (e.g., such as a dose of 3 mg/kg), about 0.9 mg/kg (e.g., such as a dose of 0.9 mg/kg), or about 0.3 mg/kg (e.g., such as a dose of 0.3 mg/kg).

Embodiment 105. The method according to Embodiment 103 or Embodiment 104, wherein the IAV infection is a H5N1 and/or a H7N9 infection.

Embodiment 106. A method of treating or preventing an influenza infection in a human subject, the method comprising administering to the subject the polynucleotide of any one of Embodiments 86-92, 99, and 100, the recombinant vector of Embodiment 93, 99, or 100, or the composition of any one of Embodiments 96-100, wherein the polynucleotide comprises mRNA.

Embodiment 107. The method of Embodiment 106, wherein the influenza infection comprises an IAV infection and/or an IBV infection.

Embodiment 108. The method of any one of Embodiments 103-107, comprising administering a single dose of the antibody or antigen-binding fragment, polypeptide, polynucleotide, recombinant vector, host cell, or composition to the subject.

Embodiment 109. The method of any one of Embodiments 103-107, comprising administering two or more doses of the antibody or antigen-binding fragment, polypeptide, polynucleotide, recombinant vector, host cell, or composition to the subject.

Embodiment 110. The method of any one of Embodiments 103-109, comprising administering a dose of the antibody or antigen-binding fragment, polypeptide, polynucleotide, recombinant vector, host cell, or composition to the subject once yearly, optionally in advance of or during an influenza season.

Embodiment 111. The method of any one of Embodiments 103-109, comprising administering a dose of the antibody or antigen-binding fragment, polypeptide, polynucleotide, recombinant vector, host cell, or composition to the subject two or more times per year; e.g. about once every 6 months.

Embodiment 112. The method of any one of Embodiments 103-111, comprising administering the antibody or antigen-binding fragment, polypeptide, polynucleotide, recombinant vector, host cell, or composition intramuscularly, subcutaneously, or intravenously.

Embodiment 113. The method of any one of Embodiments 103-112, wherein the treatment and/or prevention comprises post-exposure prophylaxis.

Embodiment 114. The method of any one of Embodiments 103-113, wherein the subject has received, is receiving, or will receive an antiviral.

Embodiment 115. The method of Embodiment 114, wherein the antiviral comprises a neuraminidase inhibitor, an influenza polymerase inhibitor, or both.

Embodiment 116. The method of Embodiment 114 or 115, wherein the antiviral comprises oseltamivir, zanamivir, baloxavir, peramivir, laninamivir, or any combination thereof.

Embodiment 117. The antibody or antigen-binding fragment of any one of Embodiments 1-85d, the polynucleotide of any one of Embodiments 86-92, 99, and 100, the recombinant vector of Embodiment 93, 99, or 100, the host cell of Embodiment 94, the human B cell of Embodiment 95, and/or the composition of any one of Embodiments 96-100, for use in a method of treating or preventing an IAV infection and/or an IBV infection in a subject.

Embodiment 118. The antibody or antigen-binding fragment of any one of Embodiments 1-85d, the polynucleotide of any one of Embodiments 86-92, 99, and 100, the recombinant vector of Embodiment 93, 99, or 100, the host cell of Embodiment 94, the human B cell of Embodiment 95, and/or the composition of any one of Embodiments 96-100, for use in the preparation of a medicament for the treatment or prevention of an IAV infection and/or an IBV infection in a subject.

Embodiment 119. A method for in vitro diagnosis of an IAV infection and/or an IBV infection, the method comprising: (i) contacting a sample from a subject with an antibody or antigen-binding fragment of any one of Embodiments 1-85d; and (ii) detecting a complex comprising an antigen and the antibody, or comprising an antigen and the antigen-binding fragment.

Embodiment 120. The method of any one of Embodiments 103, 104, 106-116, and 119 or the antibody or antigen-binding fragment, the polypeptide, the polynucleotide, the recombinant vector, the host cell, the human B cell, and/or the composition for use of any one of Embodiments 117 and 119, wherein: (i) the IAV comprises a Group 1 IAV, a Group 2 IAV, or both, wherein, optionally, the Group 1 IAV NA comprises a N1, a N4, a N5, and/or a N8; and/or the Group 2 IAV NA comprises a N2, a N3, a N6, a N7, and/or a N9, wherein, further optionally, the N1 is from A/California/07/2009, is from A/California/07/2009 I223R/H275Y, is from A/California/07/2009 Q250S, is from A/Swine/Jiangsu/J004/2018, is from A/Swine/Hebei/2017, is from A/Stockholm/18/2007, is from A/Brisbane/02/2018, is from A/Michigan/45/2015, is from A/Mississippi/3/2001, is from A/Netherlands/603/2009, is from A/Netherlands/602/2009, is from A/Vietnam/1203/2004, is from A/Vietnam/1203/2004 S247R, is from A/Vietnam/1203/2004 I223R, is from A/Vietnam/1203/2004 R152I, is from A/Vietnam/1203/2004 D199N, is from A/G4/SW/Shangdong/1207/2016, is from A/G4/SW/Henan/SN13/2018, is from A/G4/SW/Jiangsu/J004/2018, is from A/Mink/Spain/2022, and/or is from A/New Jersey/8/1976; the N4 is from A/mallard duck/Netherlands/30/2011; the N5 is from A/aquatic bird/Korea/CN5/2009; the N8 is from A/harbor seal/New Hampshire/179629/2011 and/or is from A/chicken/Russia/3-29/2020; the N2 is from A/Washington/01/2007, is from A/HongKong/68, is from A/HongKong/2671/2019, is from A/HongKong/2671/2019 K431E, is from A/South Australia/34/2019, is from A/Switzerland/8060/2017, is from A/Singapore/INFIMH-16-0019/2016, is from A/Switzerland/9715293/2013, is from A/Leningrad/134/17/57, is from A/Florida/4/2006, is from A/Netherlands/823/1992, is from A/Norway/466/2014, is from is from A/Texas/50/2012, is from A/Victoria/361/2011, is from A/SW/Mexico/SG1444/2011, is from A/Aichi/2/1968, is from A/Bilthoven/21793/1972, is from A/Netherlands/233/1982, is from A/Shanghai/11/1987, is from A/Nanchang/933/1995, is from A/Fukui/45/2004, A/Brisbane/10/2007, is from A/Tanzania/205/2010, is from A/Cambodia/2020, is from A/Perth/16/2009, is from A/Kansas/14/2017, is A/Swine/Kansas/2021, is from A/Canine/Korea/VC378/2012, and/or is from A/Canine/Indiana/003018/2016; the N3 is from A/Canada/rv504/2004 and/or is from, A/chicken/Jalisco/PAVX17170/2017; the N6 is from A/swine/Ontario/01911/1/99 is from A/Ck/Suzhou/j6/2019, and/or is from A/Hangzhou/01/2021; the N7 is from A/Netherlands/078/03 and/or is from and A/Ck/621572/03; and/or the N9 is from A/Anhui/2013, is from A/Hong Kong/56/2015; and/or (ii) the IBV NA is from: B/Lee/10/1940 (Ancestral); B/Brisbane/60/2008 (Victoria); B/Malaysia/2506/2004 (Victoria); B/Malaysia/3120318925/2013 (Yamagata); B/Wisconsin/1/2010 (Yamagata); B/Yamanashi/166/1998 (Yamagata); B/Brisbane/33/2008 (Victoria); B/Colorado/06/2017 (Victoria); B/Hubei-wujiang/158/2009 (Yamagata); B/Massachusetts/02/2012 (Yamagata); B/Netherlands/234/2011; B/Perth/211/2001 (Yamagata); B/Phuket/3073/2013 (Yamagata); B/Texas/06/2011 (Yamagata); B/HongKong/05/1972; B/Harbin/7/1994 (Victoria); B/Washington/02/2019 (Victoria); B/Perth/211/2011; B/Victoria/2/87; B/Victoria/2/87-lineage; B/Yamagata/16/88; B/Yamagata/16/88-lineage, or any combination thereof.

Embodiment 121. A method of treating or preventing an influenza infection in a subject, the method comprising administering to the subject an antibody or antigen-binding fragment at a dose of about 3 mg/kg (e.g., such as a dose of 3 mg/kg), about 0.9 mg/kg (e.g., such as a dose of 0.9 mg/kg), or about 0.3 mg/kg (e.g., such as a dose of 0.3 mg/kg), or administering to the subject a composition comprising the antibody or antigen-binding fragment at a dose of about 3 mg/kg (e.g., such as a dose of 3 mg/kg), about 0.9 mg/kg (e.g., such as a dose of 0.9 mg/kg), or about 0.3 mg/kg (e.g., such as a dose of 0.3 mg/kg), wherein: (i) the CDRH1, CDRH2, CDRH3, CDRL1, CDRL2, and CDRL3 amino acid sequences of the antibody or antigen-binding fragment are as set forth in SEQ ID NOs: 55, 4, 5, and 9-11, respectively; (ii) a VH of the antibody or antigen-binding fragment comprises the CDRH1, CDRH2, and CDRH3 of the VH amino acid sequence set forth in SEQ ID NO:54, and a VL of the antibody or antigen-binding fragment comprises the CDRL1, CDRL2, and CDRL3 of the VL amino acid sequence set forth in SEQ ID NO:8; (iii) a VH and a VL of the antibody or antigen-binding fragment comprise or consist of the amino acid sequences set forth in SEQ ID NOs:54 and 8, respectively; and/or (iv) a heavy chain and a light chain of the antibody or antigen-binding fragment comprise or consist of the amino acid sequences set forth in SEQ ID NOs: 107 (or SEQ ID NO:107 with the c-terminal lysine removed or SEQ ID NO:107 with the c-terminal glycine-lysine removed) and 108, respectively.

Embodiment 122. The method of Embodiment 121, wherein the influenza infection comprises a H5N1 IAV, a H7N9 IAV, or both.

Embodiment 123. The method of Embodiment 121 or 122, wherein the method comprises administering a single dose of the antibody or antigen-binding fragment to the subject.

Embodiment 124. The method of any one of Embodiments 121-123, wherein the method comprises administering 3 mg/kg of the antibody or antigen-binding fragment to the subject.

Embodiment 125. The method of any one of Embodiments 121-123, wherein the method comprises administering 0.9 mg/kg of the antibody or antigen-binding fragment to the subject.

Embodiment 126. The method of any one of Embodiments 121-123, wherein the method comprises administering 0.3 mg/kg of the antibody or antigen-binding fragment to the subject.

Embodiment 127. The method of any one of Embodiments 121-126, wherein the antibody or antigen-binding fragment comprises a human IgG1 isotype (e.g., comprising an allotype such as IgG1m3 or IgG1m17,1) and comprises M428L and N434S mutations in the Fc.

Embodiment 128. The method of any one of Embodiments 121-127, wherein the antibody or antigen-binding fragment comprises two heavy chains each comprising or consisting of the amino acid sequence set forth in SEQ ID NO:107 (or SEQ ID NO.: 107 with the C-terminal lysine removed or SEQ ID NO:107 with the C-terminal glycine-lysine removed) and two light chains each comprising or consisting of the amino acid sequence set forth in SEQ ID NO:108.

Embodiment 129. The method of any one of Embodiments 121-128, comprising administering the antibody, antigen-binding fragment, or composition to the subject by intravenous administration.

Embodiment 130. The method of any one of Embodiments 121-129, wherein the composition comprising the antibody or antigen-binding fragment: has an osmolality of 280-315 mOsm/kg; has no detectable endotoxin with a sensitivity <0.05 EU/mg; is sterile; has a pH of 7.4; and comprises the antibody or antigen-binding fragment purified by affinity chromatography.

Embodiment 131. The method of any one of Embodiments 121-130, wherein the subject: has an H5N1 influenza infection; is at risk of contracting an H5N1 influenza infection; has been exposed to an H5N1 influenza; has an H7N9 influenza infection; is at risk of contracting an H7N9 influenza infection; and/or or has been exposed to an H7N9 influenza.

Embodiment 132. The method of any one of Embodiments 121-131, wherein the treating or preventing comprises prophylaxis.

Embodiment 133. The method of any one of Embodiments 121-131, wherein the treating or preventing comprises post-exposure prophylaxis.

Embodiment 134. A composition comprising an anti-NA antibody or antigen-binding fragment and a pharmaceutically acceptable carrier, excipient, or diluent, wherein: (i) the antibody or antigen-binding fragment comprises CDRH1, CDRH2, CDRH3, CDRL1, CDRL2, and CDRL3 amino acid sequences as set forth in SEQ ID NOs:55, 4, 5, 9, 10, and 11, respectively; and/or (ii) the antibody or antigen-binding fragment comprises the VH and VL amino acid sequences set forth in SEQ ID NOs:54 and 8, respectively.

Embodiment 135. The composition of Embodiment 134, wherein the antibody or antigen-binding fragment comprises a human IgG1 isotype (e.g., comprising an allotype such as IgG1m3 or IgG1m17,1) and comprises M428L and N434S mutations in the Fc.

Embodiment 136. The composition of Embodiment 134 or 135, wherein the antibody or antigen-binding fragment comprises the heavy chain amino acid sequence set forth in SEQ ID NO:107 (or SEQ ID NO:107 with the C-terminal lysine removed or SEQ ID NO:107 with the C-terminal glycine-lysine removed) and the light chain amino acid sequence set forth in SEQ ID NO:108.

Embodiment 137. The composition of any one of Embodiments 134-136, wherein the antibody or antigen-binding fragment of a composition comprises two heavy chains each comprising or consisting of the amino acid sequence set forth in SEQ ID NO.: 107 (or SEQ ID NO.: 107 with the C-terminal lysine removed or SEQ ID NO.: 107 with the C-terminal glycine-lysine removed) and two light chains each comprising or consisting of the amino acid sequence set forth in SEQ ID NO:108.

Embodiment 138. The composition of any one of Embodiments 134-137, comprising the antibody or antigen-binding fragment at a concentration of 3 mg/kg, 0.9 mg/kg, or 0.3 mg/kg relative to the weight (kg) of a subject in need of the composition.

Embodiment 139. The composition of any one of Embodiments 134-138, wherein the composition: has an osmolality of 280-315 mOsm/kg; has no detectable endotoxin with a sensitivity <0.05 EU/mg; is sterile; has a pH of 7.4; and comprises the antibody or antigen-binding fragment purified by affinity chromatography.

TABLE 1
Table of Certain Sequences and Seq ID Numbers

| SEQ ID | | |
|---|---|---|
| NO | Sequence | Identifier |
| 1 | CAGGTCCACCTGGTGCAGTCTGGGGCTGAGGTGAAGGAGCC | FNI9 VH (wt-nt) |
| | TGGGTCCTCGGTGACGGTCTCCTGCAAGGCATCTGGAGGCA | |
| | GCTTCAACAACCAGGCTATTAGCTGGGTGCGACAGGCCCC | |
| | AGGACAAGGCCTTGAGTGGATGGGAGGGATCTTCCCTATC | |
| | TCTGGCACACCGACCAGCGCACAGAGGTTCCAGGGCAGAG | |
| | TCACATTTACCGCGGACGAGTCCACGACCACAGTCTACATG | |
| | GATCTGAGCAGCCTGAGATCTGACGACACGGCCGTCTACTA | |
| | CTGTGCGAGAGCGGGTTCGGATTACTTTAATAGAGACCT | |
| | CGGCTGGGAAAACTACTACTTTGCGTCCTGGGGCCAGGG | |
| | AACCCTGGTCACCGTCTCCTCAG | |
| 2 | QVHLVQSGAEVKEPGSSVTVSCKASGGSFNNQAISWVRQAPG | FNI9 VH (aa) |
| | QGLEWMGGIFPISGTPTSAQRFQGRVTFTADESTTTVYMDLSS | |
| | LRSDDTAVYYCARAGSDYFNRDLGWENYYFASWGQGTLVT | |
| | VSS | |
| 3 | GGSFNNQA | FNI9 CDRHI |
| | | (aa) |
| 4 | IFPISGTP | FNI9 CDRH2 |
| | | (aa) |
| 5 | ARAGSDYFNRDLGWENYYFAS | FNI9 CDRH3 |
| | | (aa) |
| 6 | CAGGTGCACCTGGTGCAGAGCGGAGCTGAGGTGAAGGAGCC | FNI9 VH (co-nt) |
| | AGGATCCAGCGTGACAGTGTCTTGCAAGGCTTCCGGCGGCA | |
| | GCTTCAACAATCAGGCTATCTCCTGGGTGAGGCAGGCTCCA | |
| | GGACAGGGACTGGAGTGGATGGGCGGCATCTTTCCCATCTC | |
| | TGGCACACCTACCTCCGCCCAGAGGTTCCAGGGAAGGGTGA | |
| | CCTTCACCGCTGACGAGAGCACCACAACCGTGTACATGGAT | |
| | CTGTCTTCCCTGAGATCTGACGATACCGCCGTGTACTATTGT | |
| | GCCAGAGCTGGCTCCGACTATTICAACCGCGATCTGGGCTGG | |
| | GAGAATTACTATTTTGCTTCCTGGGGCCAGGGCACACTGGTG | |
| | ACCGTGAGCTCT | |
| 7 | GAAATCGTGATGACGCAGTCTCCAGCCACCCTGTCTCTATCT | FNI9 Vk (wt-nt) |
| | TCAGGGGAAAGAGCCACCCTCTCCTGCAGGGCCAGTCGGAG | |
| | TGTTAGTAGCAACTTAGCCTGGTACCAGCAGAAACCTGGCC | |
| | AGGCTCCCAGGCTCCTCATTTATGATGCATCCACCAGGGCC | |
| | ACTGGTTTTTCAGCCAGGTTCGCTGGCAGTGGGTCTGGGACA | |
| | GAGTTCACTCTCACCATCAGCAGCCTGCAGTCTGAAGATTCT | |
| | GCTATTTATTACTGTCAGCAGTATAATAACTGGCCTCCGTG | |
| | GACGTTCGGCCAAGGGACCAAGGTGGAAATCAAAC | |
| 8 | EIVMTQSPATLSLSSGERATLSCRASRSVSSNLAWYQQKPGQAPFNI9 | VK (aa) |
| | RLLIYDASTRATGFSARFAGSGSGTEFTLTISSLQSEDSAIYYCQ | |
| | QYNNWPPWTFGQGTKVEIK | |
| 9 | RSVSSN | FNI9 CDRLI(aa) |
| 10 | DAS | FNI9 CDRL2(aa) |
| 11 | QQYNNWPPWT | FNI9 CDRL3(aa) |
| 12 | GAGATCGTGATGACCCAGTCCCCAGCCACACTGAGCCTGTC | FNI9 Vk (co-nt) |
| | CAGCGGAGAGAGGGCCACCCTGTCCTGCAGGGCTTCCCGGA | |
| | GCGTGTCTTCCAACCTGGCCTGGTACCAGCAGAAGCCAGGC | |
| | CAGGCTCCCAGACTGCTGATCTATGACGCCTCTACCAGAGCT | |
| | ACAGGCTTCTCCGCCAGGTTTGCTGGATCTGGATCCGGCACA | |
| | GAGTTCACCCTGACAATCAGCTCTCTGCAGAGCGAGGATTCT | |
| | GCTATCTACTATTGTCAGCAGTACAACAATTGGCCCCCTTGG | |
| | ACCTTTGGCCAGGGCACAAAGGTGGAGATCAAG | |
| 13 | CAGGTGCACCTGGTGCAGAGCGGAGCTGAGGTGAAGGAGCC | FNI9-VH-W110F (nt) |
| | AGGATCCAGCGTGACAGTGTCTTGCAAGGCTTCCGGCGGCA | |
| | GCTTCAACAATCAGGCTATCTCCTGGGTGAGGCAGGCTCCA | |
| | GGACAGGGACTGGAGTGGATGGGCGGCATCTTTCCCATCT | |
| | CTGGCACACCTACCTCCGCCCAGAGGTTCCAGGGAAGGGT | |
| | GACCTTCACCGCTGACGAGAGCACCACAACCGTGTACATGG | |
| | ATCTGTCTTCCCTGAGATCTGACGATACCGCCGTGTACTATT | |
| | GTGCCAGAGCTGGCTCCGACTATTTCAACCGCGATCTGG | |
| | GCTTCGAGAATTACTATTTTGCTTCCTGGGGCCAGGGCAC | |
| | ACTGGTGACCGTGAGCTCT | |
| 14 | QVHLVQSGAEVKEPGSSVTVSCKASGGSFNNQAISWVRQAPG | FNI9-VH-W110F |
| | QGLEWMGGIFPISGTPTSAQRFQGRVTFTADESTTTVYMDLSS | (aa) |
| | LRSDDTAVYYCARAGSDYFNRDLGFENYYFASWGQGTLVTV | |
| | SS | |
| 15 | ARAGSDYFNRDLGFENYYFAS | FNI9-VH-W110F |
| | | CDRH3 (aa) |
| 16 | GAGATCGTGATGACCCAGTCCCCAGCCACACTGAGCCTGTC | FNI9-VK-W94F |
| | CAGCGGAGAGAGGGCCACCCTGTCCTGCAGGGCTTCCCGGA | (nt) |
| | GCGTGTCTTCCAACCTGGCCTGGTACCAGCAGAAGCCAGG | |
| | CCAGGCTCCCAGACTGCTGATCTATGACGCCTCTACCAGAG | |
| | CTACAGGCTTCTCCGCCAGGTTTGCTGGATCTGGATCCGGCA | |
| | CAGAGTTCACCCTGACAATCAGCTCTCTGCAGAGCGAGGAT | |
| | TCTGCTATCTACTATTGTCAGCAGTACAACAATTTCCCCCC | |
| | TTGGACCTTTGGCCAGGGCACAAAGGTGGAGATCAAG | |
| 17 | EIVMTQSPATLSLSSGERATLSCRASRSVSSNLAWYQQKPGQAP | FNI9-VK-W94F |
| | RLLIYDASTRATGFSARFAGSGSGTEFTLTISSLQSEDSAIYYCQ | (aa) |
| | QYNNFPPWTFGQGTKVEIK | |
| 18 | QQYNNFPPWT | FNI9-VK-W94F |
| | | CDRL3 (aa) |
| 19 | GAGATCGTGATGACCCAGTCCCCAGCCACACTGAGCCTGTC | FNI9-VK-W97F |
| | CAGCGGAGAGAGGGCCACCCTGTCCTGCAGGGCTTCCCGGA | (nt) |
| | GCGTGTCTTCCAACCTGGCCTGGTACCAGCAGAAGCCAGG | |
| | CCAGGCTCCCAGACTGCTGATCTATGACGCCTCTACCAGAG | |
| | CTACAGGCTTCTCCGCCAGGTTTGCTGGATCTGGATCCGGCA | |
| | CAGAGTTCACCCTGACAATCAGCTCTCTGCAGAGCGAGGAT | |
| | TCTGCTATCTACTATTGTCAGCAGTACAACAATTGGCCCCC | |
| | TTTCACCTTTGGCCAGGGCACAAAGGTGGAGATCAAG | |
| 20 | EIVMTQSPATLSLSSGERATLSCRASRSVSSNLAWYQQKPGQAP | FNI9-VK-W97F |
| | RLLIYDASTRATGFSARFAGSGSGTEFTLTISSLQSEDSAIYYCQ | (aa) |
| | QYNNWPPFTFGQGTKVEIK | |
| 21 | QQYNNWPPFT | FNI9-VK-W97F |
| | | CDRL3 (aa) |
| 22 | GAGATCGTGATGACCCAGTCCCCAGCCACACTGAGCCTGTC | FNI9-VK-W94F- |
| | CAGCGGAGAGAGGGCCACCCTGTCCTGCAGGGCTTCCCGGA | W97F (nt) |
| | GCGTGTCTTCCAACCTGGCCTGGTACCAGCAGAAGCCAGG | |
| | CCAGGCTCCCAGACTGCTGATCTATGACGCCTCTACCAGAG | |
| | CTACAGGCTTCTCCGCCAGGTTTGCTGGATCTGGATCCGGCA | |
| | CAGAGTTCACCCTGACAATCAGCTCTCTGCAGAGCGAGGAT | |
| | TCTGCTATCTACTATTGTCAGCAGTACAACAATTTTCCCCC | |
| | TTTCACCTTTGGCCAGGGCACAAAGGTGGAGATCAAG | |
| 23 | EIVMTQSPATLSLSSGERATLSCRASRSVSSNLAWYQQKPGQAP | FNI9-VK-W94F- |
| | RLLIYDASTRATGFSARFAGSGSGTEFTLTISSLQSEDSAIYYCQ | W97F (aa) |
| | QYNNFPPFTFGQGTKVEIK | |
| 24 | QQYNNFPPFT | FNI9-VK-W94F- |
| | | W97F CDRL3 |
| | | (aa) |
| 25 | QVQLQQSGPGLVKPSQTLSLTCAISGDSVSSYNAVWNWIRQSP | FM08 VH |
| | SRGLEWLGRTYYRSGWYNDYAESVKSRITINPDTSKNQFSLQL | |
| | NSVTPEDTAVYYCARSGHITVFGVNVDAFDMWGQGTMVTVSS | |
| 26 | DIQMTQSPSSLSASVGDRVTITCRTSQSLSSYTHWYQQKPGKAP | FM08 VL |
| | KLLIYAASSRGSGVPSRFSGSGSGTDFTLTISSLQPEDFATYYCQ | |
| | QSRTFGQGTKVEIK | |
| 27 | APELLGGPSVFLFPPKPKDTLMISRTPEVTCVVVDVSHEDPEVK | WT hIgG1 Fc |
| | FNWYVDGVEVHNAKTKPREEQYNSTYRVVSVLTVLHQDWLN | |
| | GKEYKCKVSNKALPAPIEKTISKAKGQPREPQVYTLPPSRDELT | |
| | KNQVSLTCLVKGFYPSDIAVEWESNGQPENNYKTTPPVLDSDG | |
| | SFFLYSKLTVDKSRWQQGNVFSCSVMHEALHNHYTQKSLSLSP | |
| | GK | |
| 28 | ESKYGPPCPPCPAPPVAGP | Chimeric hinge |
| | | sequence |
| 29 | CAGGTGCACCTGGTGCAGAGCGGAGCTGAGGTGAAGGAGCC | FNI9-v5-VH (co- |
| | AGGATCCAGCGTGACAGTGTCTTGCAAGGCTTCCGGCGGCA | (nt) |
| | GCTTCAACAATCAGGCTATCTCCTGGGTGAGGCAGGCTCCA | |
| | GGACAGGGACTGGAGTGGATGGGCGGCATCTTTCCCATCTC | |
| | TGGCACACCTACCTCCGCCCAGAGGTTCCAGGGAAGGGTGA | |
| | CCTTCACCGCTGACGAGAGCACCACAACCGTGTACATGGAT | |
| | CTGTCTTCCCTGAGATCTGACGATACCGCCGTGTACTATTGT | |
| | GCCAGAGCTGGCTCCGACTATTTCAACCGCGATCTGGGCTGG | |
| | GAGAATTACTATTTTGCTTCCTGGGGCCAGGGCACACTGGTG | |
| | ACCGTGAGCTCT | |
| 30 | GAGATTGTGATGACCCAGTCCCCTGCTACCCTGAGCGTGTCC | FNI9-v5-VK (co- |
| | CCCGGAGAGAGAGCTACCCTGAGTTGCCGCGCCAGCCGCAG | nt) |
| | TGTCTCTGACAACCTGGCTTGGTACCAGCAGAAGCCAGGAC | |
| | AGGCTCCTAGGCTGCTGATCTATGGCGCCTCCACCAGGGCTA | |
| | CAGGCATCCCAGCTCGGTTCTCTGGATCCGGAAGCGGCACC | |
| | GAGTTTACCCTGACAATCTCCAGCCTGCAGAGCGAGGATTTC | |
| | GCCGTGTACTATTGCCAGCATTACAACATCTGGCCTCCTTGG | |
| | ACATTCGGTCAGGGAACTAAAGTGGAAATTAAG | |
| 31 | EIVMTQSPATLSVSPGERATLSCRASRSVSDNLAWYQQKPGQA | FNI9-v5-VK (aa) |
| | PRLLIYGASTRATGIPARFSGSGSGTEFTLTISSLQSEDFAVYYC | |
| | QHYNIWPPWTFGQGTKVEIK | |
| 32 | RSVSDN | FNI9-v5-VK |
| | | CDRLI (aa) |
| 33 | QHYNIWPPWT | FNI9-v5-VK |
| | | CDRL3 (aa) |
| 34 | ASTKGPSVFPLAPSSKSTSGGTAALGCLVKDYFPEPVTVSWNSG | IgHG1*01, G1m3 |
| | ALTSGVHTFPAVLQSSGLYSLSSVVTVPSSSLGTQTYICNVNHK | CH1-CH3 with |
| | PSNTKVDKRVEPKSCDKTHTCPPCPAPELLGGPSVFLFPPKPKD | M428L and |
| | TLMISRTPEVTCVVVDVSHEDPEVKFNWYVDGVEVHNAKTKP | N434S mutations |
| | REEQYNSTYRVVSVLTVLHQDWLNGKEYKCKVSNKALPAPIE | and C-terminal |
| | KTISKAKGQPREPQVYTLPPSREEMTKNQVSLTCLVKGFYPSDI | lysine |
| | AVEWESNGQPENNYKTTPPVLDSDGSFFLYSKLTVDKSRWQQ | |
| | GNVFSCSVLHEALHSHYTQKSLSLSPGK | |
| 35 | RTVAAPSVFIFPPSDEQLKSGTASVVCLLNNFYPREAKVQWKV | Kappa light chain |
| | DNALQSGNSQESVTEQDSKDSTYSLSSTLTLSKADYEKHKVYA | CL |
| | CEVTHQGLSSPVTKSFNRGEC | |
| 36 | ASTKGPSVFPLAPSSKSTSGGTAALGCLVKDYFPEPVTVSWNSG | IgHG1*01, G1m3 |
| | ALTSGVHTFPAVLQSSGLYSLSSVVTVPSSSLGTQTYICNVNHK | CH1-CH3 with |
| | PSNTKVDKRVEPKSCDKTHTCPPCPAPELLGGPSVFLFPPKPKD | M428L and |
| | TLMISRTPEVTCVVVDVSHEDPEVKFNWYVDGVEVHNAKTKP | N434S mutations, |
| | REEQYNSTYRVVSVLTVLHQDWLNGKEYKCKVSNKALPAPIE | without C- |
| | KTISKAKGQPREPQVYTLPPSREEMTKNQVSLTCLVKGFYPSDI | terminal lysine |
| | AVEWESNGQPENNYKTTPPVLDSDGSFFLYSKLTVDKSRWQQ | |
| | GNVFSCSVLHEALHSHYTQKSLSLSPG | |
| 37 | EIVMTQSPATLSVSPGERATLSCRASRSVSSNLAWYQQKPGQA | FNI9-variant VL |
| | PRLLIYDASTRATGIPARFSGSGSGTEFTLTISSLQSEDFAVYYC | |
| | QQYNNWPPWTFGQGTKVEIK | |
| 38 | ASTKGPSVFPLAPSSKSTSGGTAALGCLVKDYFPEPVTVSWNSG | IgHG1*01, G1m3 |
| | ALTSGVHTFPAVLQSSGLYSLSSVVTVPSSSLGTQTYICNVNHK | CH1-CH3 with |
| | PSNTKVDKRVEPKSCDKTHTCPPCPAPELLGGPSVFLFPPKPKD | M428L and |
| | TLMISRTPEVTCVVVDVSHEDPEVKFNWYVDGVEVHNAKTKP | N434A |
| | REEQYNSTYRVVSVLTVLHQDWLNGKEYKCKVSNKALPAPIE | mutations, |
| | KTISKAKGQPREPQVYTLPPSREEMTKNQVSLTCLVKGFYPSDI | without C- |
| | AVEWESNGQPENNYKTTPPVLDSDGSFFLYSKLTVDKSRWQQ | terminal lysine |
| | GNVFSCSVLHEALHAHYTQKSLSLSPG | |
| 39 | QVHLVQSGAEVKEPGSSVTVSCKASGGSFNNQAISWVRQAPG | FNI9 MLNS |
| | QGLEWMGGIFPISGTPTSAQRFQGRVTFTADESTTTVYMDLSSL | heavy chain |
| | RSDDTAVYYCARAGSDYFNRDLGWENYYFASWGQGTLVTVS | sequence, with C- |
| | SASTKGPSVFPLAPSSKSTSGGTAALGCLVKDYFPEPVTVSWNS | terminal lysine |
| | GALTSGVHTFPAVLQSSGLYSLSSVVTVPSSSLGTQTYICNVNH | |
| | KPSNTKVDKRVEPKSCDKTHTCPPCPAPELLGGPSVFLFPPKPK | |
| | DTLMISRTPEVTCVVVDVSHEDPEVKFNWYVDGVEVHNAKTK | |
| | PREEQYNSTYRVVSVLTVLHQDWLNGKEYKCKVSNKALPAPIE | |
| | KTISKAKGQPREPQVYTLPPSREEMTKNQVSLTCLVKGFYPSDI | |
| | AVEWESNGQPENNYKTTPPVLDSDGSFFLYSKLTVDKSRWQQ | |
| | GNVFSCSVLHEALHSHYTQKSLSLSPGK | |
| 40 | QVHLVQSGAEVKEPGSSVTVSCKASGGSFNNQAISWVRQAPG | FNI9_MLNA |
| | QGLEWMGGIFPISGTPTSAQRFQGRVTFTADESTTTVYMDLSSL | heavy chain |
| | RSDDTAVYYCARAGSDYFNRDLGWENYYFASWGQGTLVTVS | sequence, with C- |
| | SASTKGPSVFPLAPSSKSTSGGTAALGCLVKDYFPEPVTVSWNS | terminal lysine |
| | GALTSGVHTFPAVLQSSGLYSLSSVVTVPSSSLGTQTYICNVNH | |
| | KPSNTKVDKRVEPKSCDKTHTCPPCPAPELLGGPSVFLFPPKPK | |
| | DTLMISRTPEVTCVVVDVSHEDPEVKFNWYVDGVEVHNAKTK | |
| | PREEQYNSTYRVVSVLTVLHQDWLNGKEYKCKVSNKALPAPIE | |
| | KTISKAKGQPREPQVYTLPPSREEMTKNQVSLTCLVKGFYPSDI | |
| | AVEWESNGQPENNYKTTPPVLDSDGSFFLYSKLTVDKSRWQQ | |
| | GNVFSCSVLHEALHAHYTQKSLSLSPGK | |
| 41 | EIVMTQSPATLSLSSGERATLSCRASRSVSSNLAWYQQKPGQAP | FNI9 light chain |
| | RLLIYDASTRATGFSARFAGSGSGTEFTLTISSLQSEDSAIYYCQ | sequence |
| | QYNNWPPWTFGQGTKVEIKRTVAAPSVFIFPPSDEQLKSGTAS | |
| | VVCLLNNFYPREAKVQWKVDNALQSGNSQESVTEQDSKDSTY | |
| | SLSSTLTLSKADYEKHKVYACEVTHQGLSSPVTKSFNRGEC | |
| 42 | QVHLVQSGAEVKEPGSSVTVSCKASGGSFNNQAISWVRQAPG | FNI9- |
| | QGLEWMGGIFPISGTPTSAQRFQGRVTFTADESTTTVYMDLSS | variant_MLNS |
| | LRSDDTAVYYCARAGSDYFNRDLGWENYYFASWGQGTLVT | heavy chain |
| | VSSASTKGPSVFPLAPSSKSTSGGTAALGCLVKDYFPEPVTVSW | sequence, with C- |
| | NSGALTSGVHTFPAVLQSSGLYSLSSVVTVPSSSLGTQTYICNV | terminal lysine |
| | NHKPSNTKVDKRVEPKSCDKTHTCPPCPAPELLGGPSVFLFPPK | |
| | PKDTLMISRTPEVTCVVVDVSHEDPEVKFNWYVDGVEVHNAK | |
| | TKPREEQYNSTYRVVSVLTVLHQDWLNGKEYKCKVSNKALPA | |
| | PIEKTISKAKGQPREPQVYTLPPSREEMTKNQVSLTCLVKGFYP | |
| | SDIAVEWESNGQPENNYKTTPPVLDSDGSFFLYSKLTVDKSRW | |
| | QQGNVFSCSVLHEALHSHYTQKSLSLSPGK | |
| 43 | QVHLVQSGAEVKEPGSSVTVSCKASGGSFNNQAISWVRQAPG | FNI9- |
| | QGLEWMGGIFPISGTPTSAQRFQGRVTFTADESTTTVYMDLSS | variant_MLNA |
| | LRSDDTAVYYCARAGSDYFNRDLGWENYYFASWGQGTLVT | heavy chain |
| | VSSASTKGPSVFPLAPSSKSTSGGTAALGCLVKDYFPEPVTVSW | sequence, with C- |
| | NSGALTSGVHTFPAVLQSSGLYSLSSVVTVPSSSLGTQTYICNV | terminal lysine |
| | NHKPSNTKVDKRVEPKSCDKTHTCPPCPAPELLGGPSVFLFPPK | |
| | PKDTLMISRTPEVTCVVVDVSHEDPEVKFNWYVDGVEVHNAK | |
| | TKPREEQYNSTYRVVSVLTVLHQDWLNGKEYKCKVSNKALPA | |
| | PIEKTISKAKGQPREPQVYTLPPSREEMTKNQVSLTCLVKGFYP | |
| | SDIAVEWESNGQPENNYKTTPPVLDSDGSFFLYSKLTVDKSRW | |
| | QQGNVFSCSVLHEALHAHYTQKSLSLSPGK | |
| 44 | EIVMTQSPATLSVSPGERATLSCRASRSVSSNLAWYQQKPGQA | FNI9-variant light |
| | PRLLIYDASTRATGIPARFSGSGSGTEFTLTISSLQSEDFAVYYC | chain sequence |
| | QQYNNWPPWTFGQGTKVEIKRTVAAPSVFIFPPSDEQLKSGTA | |
| | SVVCLLNNFYPREAKVQWKVDNALQSGNSQESVTEQDSKDST | |
| | YSLSSTLTLSKADYEKHKVYACEVTHQGLSSPVTKSFNRGEC | |
| 45 | QVQLVQSGAEVKKPGSSVKVSCKASGGSFNNQAISWVRQAPG | FNI9-VH- |
| | QGLEWMGGIFPISGTPTSAQRFQGRVTFTADESTTTVYMDLSS | FR124GL |
| | LRSDDTAVYYCARAGSDYFNRDLGWENYYFASWGQGTLVT | |
| | VSS | |
| 46 | QVHLVQSGAEVKEPGSSVTVSCKASGGSFSNQAISWVRQAPG | FNI9-VH.4 |
| | QGLEWMGGIFPISGTPTSAQRFQGRVTFTADESTTTVYMDLSS | |
| | LRSDDTAVYYCARAGSDYFNRDLGWENYYFASWGQGTLVT | |
| | VSS | |
| 47 | GGSFSNQA | FNI9-VH.4 |
| | | CDRH1 |
| 48 | QVHLVQSGAEVKEPGSSVTVSCKASGGSFNKQAISWVRQAPG | FNI9-VH.5 |
| | QGLEWMGGIFPISGTPTSAQRFQGRVTFTADESTTTVYMDLSS | |
| | LRSDDTAVYYCARAGSDYFNRDLGWENYYFASWGQGTLVT | |
| | VSS | |
| 49 | GGSFNKQA | FNI9-VH.5 |
| | | CDRH1 |
| 50 | QVHLVQSGAEVKEPGSSVTVSCKASGGSFNNQAISWVRQAPG | FNI9-VH.6 |
| | QGLEWMGGIFPISGTPTSAQRFQGRVTFTADESTTTVYMDLSS | |
| | LRSDDTAVYYCARAGSEYFNRDLGWENYYFASWGQGTLVT | |
| | VSS | |
| 51 | ARAGSEYFNRDLGWENYYFAS | FNI9-VH.6 |
| | | CDRH3 |
| 52 | QVHLVQSGAEVKEPGSSVTVSCKASGGSFNNQAISWVRQAPG | FNI9-VH.7 |
| | QGLEWMGGIFPISGTPTSAQRFQGRVTFTADESTTTVYMDLSS | |
| | LRSDDTAVYYCARAGSDYFNRDLGWEQYYFASWGQGTLVT | |
| | VSS | |
| 53 | ARAGSDYFNRDLGWEQYYFAS | FNI9-VH.7 |
| | | CDRH3 |
| 54 | QVHLVQSGAEVKEPGSSVTVSCKASGGTFNNQAISWVRQAPG | FNI9-VH.8 |
| | QGLEWMGGIFPISGTPTSAQRFQGRVTFTADESTTTVYMDLSS | |
| | LRSDDTAVYYCARAGSDYFNRDLGWENYYFASWGQGTLVT | |
| | VSS | |
| 55 | GGTFNNQA | FNI9-VH.8 |
| | | CDRH1 |
| 56 | QVHLVQSGAEVKEPGSSVTVSCKASGGSFNNQAISWVRQAPG | FNI9-VH.9 |
| | QGLEWMGGIFPISGTATSAQRFQGRVTITADESTTTVYMDLSS | |
| | LRSDDTAVYYCARAGSDYFNRDLGWENYYFASWGQGTLVT | |
| | VSS | |
| 57 | IFPISGTA | FNI9-VH.9 |
| | | CDRH2 |
| 58 | QVHLVQSGAEVKEPGSSVTVSCKASGGSFNNQAISWVRQAPG | FNI9-VH.10 |
| | QGLEWMGGIFPISGTPTYAQRFQGRVTITADESTTTVYMDLSS | |
| | LRSDDTAVYYCARAGSDYFNRDLGWENYYFASWGQGTLVT | |
| | VSS | |
| 59 | IFPISGTPTY | FNI9-VH.10 |
| | | CDRH2 and two |
| | | C-terminal |
| | | flanking amino |
| | | acids |
| 60 | QVHLVQSGAEVKEPGSSVTVSCKASGGSFNNQAISWVRQAPG | FNI9-VH.11 |
| | QGLEWMGGIFPISGTATYAQRFQGRVTITADESTTTVYMDLSS | |
| | LRSDDTAVYYCARAGSDYFNRDLGWENYYFASWGQGTLVT | |
| | VSS | |
| 61 | IFPISGTA | FNI9-VH.11 |
| | | CDRH2 |
| 62 | IFPISGTATY | FNI9-VH.11 |
| | | CDRH2 and two |
| | | C-terminal |
| | | flanking amino |
| | | acids |
| 63 | QVHLVQSGAEVKEPGSSVTVSCKASGGSFNNQAISWVRQAPG | FNI9-VH.12 |
| | QGLEWMGGIFPISGTANYAQRFQGRVTITADESTTTVYMDLSS | |
| | LRSDDTAVYYCARAGSDYFNRDLGWENYYFASWGQGTLVT | |
| | VSS | |
| 64 | IFPISGTANY | FNI9-VH.12 |
| | | CDRH2 and two |
| | | C-terminal |
| | | flanking amino |
| | | acids |
| 65 | QVQLVQSGAEVKEPGSSVTVSCKASGGSFNNQAISWVRQAPG | FNI9-VH. 13 |
| | QGLEWMGGIFPISGTPTSAQRFQGRVTFTADESTTTVYMDLSS | |
| | LRSDDTAVYYCARAGSDYFNRDLGWENYYFASWGQGTLVT | |
| | VSS | |
| 66 | EIVMTQSPATLSLSSGERATLSCRASRSVSSNLAWYQQKPGQAP | FNI9-VK.7 |
| | RLLIYDASTRATGFSARFAGSGSGTEFTLTISSLQSEDSAIYYCQ | |
| | QYNIWPPWTFGQGTKVEIK | |
| 67 | QQYNIWPPWT | FNI9-VK.7 |
| | | CDRL3 |
| 68 | EIVMTQSPATLSVSPGERATLSCRASRSVSSNLAWYQQKPGQA | FNI9-VK.8 |
| | PRLLIYDASTRATGFSARFAGSGSGTEFTLTISSLQSEDSAIYYC | |
| | QQYNNWPPWTFGQGTKVEIK | |
| 69 | ASTKGPSVFPLAPSSKSTSGGTAALGCLVKDYFPEPV | Wild-type human |
| | TVSWNSGALTSGVHTFPAVLQSSGLYSLSSVVTVPSS | IgG1 CH1-CH3 |
| | SLGTQTYICNVNHKPSNTKVDKKVEPKSCDKTHTCPP | (UniProtKB |
| | CPAPELLGGPSVFLFPPKPKDTLMISRTPEVTCVVVDV | P01857); hinge |
| | SHEDPEVKFNWYVDGVEVHNAKTKPREEQYNSTYRV | underlined, CH2 |
| | VSVLTVLHQDWLNGKEYKCKVSNKALPAPIEKTISKA | italicized |
| | KGQPREPQVYTLPPSRDELTKNQVSLTCLVKGFYPSD | |
| | IAVEWESNGQPENNYKTTPPVLDSDGSFFLYSKLTVD | |
| | KSRWQQGNVFSCSVMHEALHNHYTQKSLSLSPGK | |
| 70 | EPKSCDKTHTCPPCPAPELLGGPSVFLFPPKPKDTLMISRTPEVTC | Wild-type human |
| | VVVDVSHEDPEVKFNWYVDGVEVHNAKTKPREEQYNSTYRVVSVL | IgG1 hinge-CH2- |
| | TVLHQDWLNGKEYKCKVSNKALPAPIEKTISKAKGQPREPQVYTL | CH3; hinge |
| | PPSRDELTKNQVSLTCLVKGFYPSDIAVEWESNGQPENNYKTTP | underlined, CH2 |
| | PVLDSDGSFFLYSKLTVDKSRWQQGNVFSCSVMHEALHNHYT | italicized |
| | QKSLSLSPGK | |
| 71 | APELLGGPSVFLFPPKPKDTLMISRTPEVTCVVVDVSHEDPEVK | Wild-type human |
| | FNWYVDGVEVHNAKTKPREEQYNSTYRVVSVLTVLHQDWLN | IgG1 CH2 |
| | GKEYKCKVSNKALPAPIEKTISKAK | |
| 72 | GQPREPQVYTLPPSRDELTKNQVSLTCLVKGFYPSDIAVEWESN | Wild-type human |
| | GQPENNYKTTPPVLDSDGSFFLYSKLTVDKSRWQQGNVFSCSV | IgG1 CH3 |
| | MHEALHNHYTQKSLSLSPGK | |
| 73 | EPKSCDKTHTCPPCPAPELLGGPSVFLFPPKPKDTLMISRTPEVTC | Wild-type human |
| | VVVDVSHEDPEVKFNWYVDGVEVHNAKTKPREEQYNSTYRVVSVL | IgG1 hinge-CH2; |
| | TVLHQDWLNGKEYKCKVSNKALPAPIEKTISKAK | hinge underlined, |
| | | CH2 italicized |
| 74 | APELLAGPSVFLFPPKPKDTLMISRTPEVTCVVVDVSHEDPEVK | Human IgG1 |
| | FNWYVDGVEVHNAKTKPREEEYNSTYRVVSVLTVLHQDWLN | CH2-CH3 amino |
| | GKEYKCKVSNKAVPAPIEKTISKAKGQPREPQVYTLPPSRDELT | acid sequence |
| | KNQVSLTCLVKGFYPSDIAVEWESNGQPENNYKTTPPVLDSDG | with G236A, |
| | SFFLYSKLTVDKSRWQQGNVFSCSVMHEALHNHYTQKSLSLSP | L328V, and |
| | GK | Q295E mutations |
| 75 | EPKSCDKTHTCPPCAAPELLAGPSVFLFPPKPKDTLMISRTPEVT | Human IgG1 |
| | CVVVDVSHEDPEVKFNWYVDGVEVHNAKTKPREEEYNSTYR | hinge-CH2-CH3 |
| | VVSVLTVLHQDWLNGKEYKCKVSNKALPAPIEKTISKAKGQPR | amino acid |
| | EPQVYTLPPSRDELTKNQVSLTCLVKGFYPSDIAVEWESNGQPE | sequence with |
| | NNYKTTPPVLDSDGSFFLYSKLTVDKSRWQQGNVFSCSVMHE | G236A, P230A, |
| | ALHNHYTQKSLSLSPGK | and Q295E |
| | | mutations |
| 76 | APELLAGPSVFLFPPKPKDTLMISRTPEVTCVVVDVSHEDPEVK | Human IgG1 |
| | FNWYVDGVEVHNAKTKPPEEQYNSTYRVVSVLTVLHQDWLN | CH2-CH3 amino |
| | GKEYKCKVSNKALPAPIEKTISKAKGQPREPQVYTLPPSRDELT | acid sequence |
| | KNQVSLTCLVKGFYPSDNAVEWESNGQPENNYKTTPPVLDSD | with G236A, |
| | GSFFLYSKLTVDKSRWQQGNVFSCSVMHEALHNHYTQKSLSLS | R292P, and |
| | PGK | I377N mutations |
| 77 | APELLAGPSVFLFPPKPKDTLMISRTPEVTCVVVDVSHEDPEVK | Human IgG1 |
| | FNWYVDGVEVHNAKTKPREEEYNSTYRVVSVLTVLHQDWLN | CH2-CH3 amino |
| | GKEYKCKVSNKALPAPIEATISKAKGQPREPQVYTLPPSRDELT | acid sequence |
| | KNQVSLTCLVKGFYPSDIAVEWESNGQPENNYKTTPPVLDSDG | with G236A, |
| | SFFLYSKLTVDKSRWQQGNVFSCSVMHEALHNHYTQKSLSLSP | K334A, and |
| | GK | Q295E mutations |
| 78 | APELLSGPSVFLFPPKPKDTLMISRTPEVTCVVVDVSHEDPEVKF | Human IgG1 |
| | NWYVDGVEVHNAKTKPPEEQYNSTLRVVSVLTVLHQDWLNG | CH2-CH3 amino |
| | KEYKCKVSNKALPAPIEKTISKAKGQPREPQVYTLPPSRDELTK | acid sequence |
| | NQVSLTCLVKGFYPSDIAVEWESNGQPENNYKTTPPVLDSDGS | with G236S, |
| | FFLYSKLTVDKSRWQQGNVFSCSVMHEALHNHYTQKSLSLSPG | R292P, and |
| | K | Y300L mutations |
| 79 | APELLAGPSVFLFPPKPKDTLMISRTPEVTCVVVDVSHEDPEVK | Human IgG1 |
| | FNWYVDGVEVHNAKTKPREEQYNSTLRVVSVLTVLHQDWLN | CH2-CH3 amino |
| | GKEYKCKVSNKALPAPIEKTISKAKGQPREPQVYTLPPSRDELT | acid sequence |
| | KNQVSLTCLVKGFYPSDIAVEWESNGQPENNYKTTPPVLDSDG | with G236A and |
| | SFFLYSKLTVDKSRWQQGNVFSCSVMHEALHNHYTQKSLSLSP | Y300L mutations |
| | GK | |
| 80 | APELLAGPSVFLFPPKPKDTLMISRTPEVTCVVVDVSHEDPEVK | Human IgG1 |
| | FNWYVDGVEVHNAKTKPPEEQYNSTLRVVSVLTVLHQDWLN | CH2-CH3 amino |
| | GKEYKCKVSNKALPAPIEKTISKAKGQPREPQVYTLPPSRDELT | acid sequence |
| | KNQVSLTCLVKGFYPSDIAVEWESNGQPENNYKTTPPVLDSDG | with G236A, |
| | SFFLYSKLTVDKSRWQQGNVFSCSVMHEALHNHYTQKSLSLSP | R292P, and |
| | GK | Y300L mutations |
| 81 | APELLSGPSVFLFPPKPKDTLMISRTPEVTCVVVDVSHEDPEVKF | Human IgG1 |
| | NWYVDGVEVHNAKTKPREEQYNSTYRVVSVLTVTHQDWLNG | CH2-CH3 amino |
| | KEYKCKVSNKALPAPIEKTISKAKGQPREPQVYTLPPSRDELTK | acid sequence |
| | NQVSLTCLVKGFYPSDIAVEWESNGQPENNYKTTPPVLDSDGS | with G236S, |
| | FFLYSKLTVDKSRWQQVNVFSCSVMHEALHNHYTQKSLSLSPE | G420V, G446E, |
| | K | and L309T |
| | | mutations |
| 82 | APELLAGPSVFLFPPKPKDTLMISRTPEVTCVVVDVSHEDPEVK | Human IgG1 |
| | FNWYVDGVEVHNAKTKPPEEQYNSTYRVVSVLTVLHQDWLN | CH2-CH3 amino |
| | GKEYKCKVSNKALPAPIEKTISKAKGQPREPQVYTLPPSRDELT | acid sequence |
| | KNQVSLTCLVKGFYPSDIAVEWESNGQPENNYKTTPPVLDSDG | with G236A and |
| | SFFLYSKLTVDKSRWQQGNVFSCSVMHEALHNHYTQKSLSLSP | R292P mutations |
| | GK | |
| 83 | APELLGGPSVFLFPPKPKDTLMISRTPEVTCVVVDVSHEDPEVK | Human IgG1 |
| | FNWYVDGVEVHNAKTKPPEEQYNSTLRVVSVLTVLHQDWLN | CH2-CH3 amino |
| | GKEYKCKVSNKALPAPIEKTISKAKGQPREPQVYTLPPSRDELT | acid sequence |
| | KNQVSLTCLVKGFYPSDIAVEWESNGQPENNYKTTPPVLDSDG | with R292P and |
| | SFFLYSKLTVDKSRWQQGNVFSCSVMHEALHNHYTQKSLSLSP | Y300L mutations |
| | GK | |
| 84 | APELLAGPSVFLFPPKPKDTLMISRTPEVTCVVVDVSHEDPEVK | Human IgG1 |
| | FNWYVDGVEVHNAKTKPPEEQYNSTYRVVSVLTVLHQDWLN | CH2-CH3 amino |
| | GKEYKCKVSNKALPAPIEKTISKAKGQPREPQVYTLPPSRDELT | acid sequence |
| | KNQVSLTCLVKGFYPSDIAVEWESNGQPENNYKTTPPVLDSDG | with G236A and |
| | SFFLYSKLTVDKSRWQQGNVFSCSVMHEALHNHYTQKSLSLSP | R292P mutations |
| | GK | |
| 85 | APELLGGPSVFLFPPKPKDTLMISRTPEVTCVVVDVSHEDPEVK | Human IgG1 |
| | FNWYVDGVEVHNAKTKPREEQYNSTLRVVSVLTVLHQDWLN | CH2-CH3 amino |
| | GKEYKCKVSNKALPAPIEKTISKAKGQPREPQVYTLPPSRDELT | acid sequence |
| | KNQVSLTCLVKGFYPSDIAVEWESNGQPENNYKTTPPVLDSDG | with Y300L |
| | SFFLYSKLTVDKSRWQQGNVFSCSVMHEALHNHYTQKSLSLSP | mutation |
| | GK | |
| 86 | APELYSGPSVFLFPPKPKDTLMISRTPEVTCVVVDVEHEDPEVK | Human IgG1 |
| | FNWYVDGVEVHNAKTKPREEQYNSTYRVVSVLTVLHQDWLN | CH2-CH3 amino |
| | GKEYKCKVSNKALPAPIEKTISKAKGQPRKPQVYTLPPSRDELT | acid sequence |
| | KNQVSLTCLVKGFYPSDIAVEWESNGQPENNYKTTPPVLDSDG | with E345K, |
| | SFFLYSKLTVDKSRWQQGNVFSCSVMHEALHNHYTQKSLSLSP | G236S, L235Y, |
| | GK | and S267E |
| | | mutations |
| 87 | EPKYCDKTHTCPPCPAPELLGGPSVFLFPPKPKDTLMISRTPEVT | Human IgG1 |
| | CVVVDVEHEDPRVKFNWYVDGVEVHNAKTKPREEQYNSTYR | hinge-CH2-CH3 |
| | VVSVLTVTHQDWLNGKEYKCKVSNKALPAPIEKTISKAKGQPR | amino acid |
| | EPQVYTLPPSRDELTKNQVSLTCLVKGFYPSDIAVEWESNGQPE | sequence with |
| | NNYKTTPPVLDSDGSFFLYSKLTVDKSRWQQGNVFSCSVMHE | E272R, L309T, |
| | ALHNHYTQKSLSLSPGK | S219Y, and |
| | | S267E mutations |
| 88 | APELLYGPSVFLFPPKPKDTLMISRTPEVTCVVVDVSHEDPEVK | Human IgG1 |
| | FNWYVDGVEVHNAKTKPREEQYNSTYRVVSVLTVLHQDWLN | CH2-CH3 amino |
| | GKEYKCKVSNKALPAPIEKTISKAKGQPREPQVYTLPPSRDELT | acid sequence |
| | KNQVSLTCLVKGFYPSDIAVEWESNGQPENNYKTTPPVLDSDG | with G236Y |
| | SFFLYSKLTVDKSRWQQGNVFSCSVMHEALHNHYTQKSLSLSP | mutation |
| | GK | |
| 89 | APELLWGPSVFLFPPKPKDTLMISRTPEVTCVVVDVSHEDPEVK | Human IgG1 |
| | FNWYVDGVEVHNAKTKPREEQYNSTYRVVSVLTVLHQDWLN | CH2-CH3 amino |
| | GKEYKCKVSNKALPAPIEKTISKAKGQPREPQVYTLPPSRDELT | acid sequence |
| | KNQVSLTCLVKGFYPSDIAVEWESNGQPENNYKTTPPVLDSDG | with G236W |
| | SFFLYSKLTVDKSRWQQGNVFSCSVMHEALHNHYTQKSLSLSP | mutation |
| | GK | |
| 90 | APELLGGPSVFLLPPKPKDTLMISRTPEVTCVVVDVEHEDPEVK | Human IgG1 |
| | FNWYVDGVEVHNAKTKPREEQYNSTYRVVSVLTVLHQDWLN | CH2-CH3 amino |
| | GKEYKCKVSNKALPAPIEKTISKAKGQPREPQVYTLPPSRDELT | acid sequence |
| | KNQVSLTCLVKGFYPSDIAVEWESNGQPENNYKTTPLVLDSDG | with F243L, |
| | SFFLYSKLTVDKSRWQQGNVFSCSVMHEALHNHYTQKSLSLSP | G446E, P396L, |
| | EK | and S267E |
| | | mutations |
| 91 | APELLAGPSVFLFPPKPKDTLMISRTPEVTCVVVDVSHEDPEVK | Human IgG1 |
| | FNWYVDGVEVHNAKTKPREEQYNSTYRVVSVLTVLHQDWLN | CH2-CH3 amino |
| | GKEYKCKVSNKALPAPIEKTISKAKGQPREPQVYTLPPSRDELT | acid sequence |
| | KNQVSLTCLVKGFYPSDIAVEWESNGQPENNYKTTPPVLDSDG | with G236A |
| | SFFLYSKLTVDKSRWQQGNVFSCSVMHEALHNHYTQKSLSLSP | mutation |
| | GK | |
| 92 | APELLAGPDVFLFPPKPKDTLMISRTPEVTCVVVDVSEEDPEVK | Human IgG1 |
| | FNWYVDGVEVHNAKTKPREEQYNSTYRVVSVLTVLHQDWLN | CH2-CH3 amino |
| | GKEYKCKVSNKALPAPIEKTISKAKGQPREPQVYTLPPSRDELT | acid sequence |
| | KNQVSLTCLVKGFYPSDIAVEWESNGQPENNYKTTPPVLDSDG | with G236A, |
| | SFFLYSKLTVDKSRWQQGNVFSCSVMHEALHNHYTQKSLSLSP | S239D, and |
| | GK | H268E mutations |
| 93 | ASTKGPSVFPLAPSSKSTSGGTAALGCLVKDYFPEPVTVSWNSG | IgG1m3 CH1- |
| | ALTSGVHTFPAVLQSSGLYSLSSVVTVPSSSLGTQTYICNVNHK | CH3 |
| | PSNTKVDKRVEPKSCDKTHTCPPCPAPELLGGPSVFLFPPKPKD | |
| | TLMISRTPEVTCVVVDVSHEDPEVKFNWYVDGVEVHNAKTKP | |
| | REEQYNSTYRVVSVLTVLHQDWLNGKEYKCKVSNKALPAPIE | |
| | KTISKAKGQPREPQVYTLPPSREEMTKNQVSLTCLVKGFYPSDI | |
| | AVEWESNGQPENNYKTTPPVLDSDGSFFLYSKLTVDKSRWQQ | |
| | GNVFSCSVMHEALHNHYTQKSLSLSPGK | |
| 94 | ASTKGPSVFPLAPSSKSTSGGTAALGCLVKDYFPEPVTVSWNSG | IgG1m17, 1 CH1- |
| | ALTSGVHTFPAVLQSSGLYSLSSVVTVPSSSLGTQTYICNVNHK | CH3 |
| | PSNTKVDKKVEPKSCDKTHTCPPCPAPELLGGPSVFLFPPKPKD | |
| | TLMISRTPEVTCVVVDVSHEDPEVKFNWYVDGVEVHNAKTKP | |
| | REEQYNSTYRVVSVLTVLHQDWLNGKEYKCKVSNKALPAPIE | |
| | KTISKAKGQPREPQVYTLPPSRDELTKNQVSLTCLVKGFYPSDI | |
| | AVEWESNGQPENNYKTTPPVLDSDGSFFLYSKLTVDKSRWQQ | |
| | GNVFSCSVMHEALHNHYTQKSLSLSPGK | |
| 95 | ASTKGPSVFPLAPSSKSTSGGTAALGCLVKDYFPEPVTVSWNSG | IgG1m3, 1 CH1- |
| | ALTSGVHTFPAVLQSSGLYSLSSVVTVPSSSLGTQTYICNVNHK | CH3 |
| | PSNTKVDKRVEPKSCDKTHTCPPCPAPELLGGPSVFLFPPKPKD | |
| | TLMISRTPEVTCVVVDVSHEDPEVKFNWYVDGVEVHNAKTKP | |
| | REEQYNSTYRVVSVLTVLHQDWLNGKEYKCKVSNKALPAPIE | |
| | KTISKAKGQPREPQVYTLPPSRDELTKNQVSLTCLVKGFYPSDI | |
| | AVEWESNGQPENNYKTTPPVLDSDGSFFLYSKLTVDKSRWQQ | |
| | GNVFSCSVMHEALHNHYTQKSLSLSPGK | |
| 96 | GAS | FNI9-v5 CDRL2 |
| | | (aa) |
| 97 | caggtgcagctggtgcagtctggcgccgaggtgaagaagccaggctccagcgtga | FNI-UCA-IGH |
| | aggtgagctgcaaggcttctggcggcaccttctcttcctacgctatctcctgggt | (wt-nt) |
| | gaggcaggctccaggacagggactggagtggatgggcggcatcatccctatcttc | |
| | ggcacagccaactacgctcagaagtttcagggcagagtgaccatcacagccgacg | |
| | agtctacctccacagcttatatggagctgagctctctgcgctccgaggataccgc | |
| | cgtgtactattgtgccagggctggcagcgactacttcaaccgggatctgggctgg | |
| | gagaattactattttgactattggggccagggcaccctggtgacagtgtccagc | |
| 98 | QVQLVQSGAEVKKPGSSVKVSCKASGGTFSSYAISWVRQAPGQ | FNI-UCA VH |
| | GLEWMGGIIPIFGTANYAQKFQGRVTITADESTSTAYMELSSLR | (aa) |
| | SEDTAVYYCARAGSDYFNRDLGWENYYFDYWGQGTLVTVSS | |
| 99 | gagatcgtgatgacccagtctcctgccacactgagcgtgtctccaggagagaggg | FNI-UCA-IGK |
| | ccaccctgtcctgcagggcttcccagagcgtgtccagcaacctggcctggtacca | (wt-nt) |
| | gcagaagccaggccaggctcccaggctgctgatctatggcgccagcaccagagct | |
| | acaggcatcccagctcgcttctctggatccggaagcggcacagagtttaccctga | |
| | caatctcttccctgcagtctgaggacttcgccgtgtactattgtcagcagtacaa | |
| | caattggcccccttggacctttggccagggcacaaaggtggagatcaag | |
| 100 | EIVMTQSPATLSVSPGERATLSCRASQSVSSNLAWYQQKPGQA | FNI-UCA VK |
| | PRLLIYGASTRATGIPARFSGSGSGTEFTLTISSLQSEDFAVYYCQ(aa) | |
| | QYNNWPPWTFGQGTKVEIK | |
| 101 | GGTFSSYA | FNI-UCA |
| | | CDRHI |
| 102 | IIPIFGTA | FNI-UCA |
| | | CDRH2 |
| 103 | ARAGSDYFNRDLGWENYYFDY | FNI-UCA |
| | | CDRH3 |
| 104 | QSVSSN | FNI-UCA |
| | | CDRL1 |
| 105 | GAS | FNI-UCA |
| | | CDRL2 |
| 106 | QQYNNWPPWT | FNI-UCA |
| | | CDRL3 |
| 107 | QVHLVQSGAEVKEPGSSVTVSCKASGGTFNNQAISWVRQAPG | FNI9-v8.1 heavy |
| | QGLEWMGGIFPISGTPTSAQRFQGRVTFTADESTTTVYMDLSSL | chain amino acid |
| | RSDDTAVYYCARAGSDYFNRDLGWENYYFASWGQGTLVTVS | (IgG1m3 with |
| | SASTKGPSVFPLAPSSKSTSGGTAALGCLVKDYFPEPVTVSWNS | M428L and |
| | GALTSGVHTFPAVLQSSGLYSLSSVVTVPSSSLGTQTYICNVNH | N434S mutations |
| | KPSNTKVDKRVEPKSCDKTHTCPPCPAPELLGGPSVFLFPPKPK | in CH3) |
| | DTLMISRTPEVTCVVVDVSHEDPEVKFNWYVDGVEVHNAKTK | |
| | PREEQYNSTYRVVSVLTVLHQDWLNGKEYKCKVSNKALPAPIE | |
| | KTISKAKGQPREPQVYTLPPSREEMTKNQVSLTCLVKGFYPSDI | |
| | AVEWESNGQPENNYKTTPPVLDSDGSFFLYSKLTVDKSRWQQ | |
| | GNVFSCSVLHEALHSHYTQKSLSLSPGK | |
| 108 | EIVMTQSPATLSLSSGERATLSCRASRSVSSNLAWYQQKPGQAP | FN19-v8.1 light |
| | RLLIYDASTRATGFSARFAGSGSGTEFTLTISSLQSEDSAIYYCQ | chain amino acid |
| | QYNNWPPWTFGQGTKVEIKRTVAAPSVFIFPPSDEQLKSGTASV | |
| | VCLLNNFYPREAKVQWKVDNALQSGNSQESVTEQDSKDSTYS | |
| | LSSTLTLSKADYEKHKVYACEVTHQGLSSPVTKSFNRGEC | |
| 109 | CAGGTGCACCTGGTGCAGAGCGGAGCTGAGGTGAAGGAGCC | FNI9-v8.1 heavy |
| | AGGATCCAGCGTGACAGTGTCTTGCAAGGCTTCCGGCGGCA | chain nucleotide |
| | CCTTCAACAATCAGGCTATCTCCTGGGTGAGGCAGGCTCCAG | (IgG1m3 with |
| | GACAGGGACTGGAGTGGATGGGCGGCATCTTTCCCATCTCT | M428L and |
| | GGCACACCTACCTCCGCCCAGAGGTTCCAGGGAAGGGTGAC | N434S mutations |
| | CTTCACCGCTGACGAGAGCACCACAACCGTGTACATGGATC | in CH3) |
| | TGTCTTCCCTGAGATCTGACGATACCGCCGTGTACTATTGTG | |
| | CCAGAGCTGGCTCCGACTATTTCAACCGCGATCTGGGCTGGG | |
| | AGAATTACTATTTTGCTTCCTGGGGCCAGGGCACACTGGTGA | |
| | CCGTGAGCTCTGCGTCGACCAAGGGCCCATCGGTCTTCCCCC | |
| | TGGCACCAAGTAGCAAGAGCACATCCGGTGGCACAGCCGCC | |
| | CTGGGTTGTCTGGTGAAAGATTATTTCCCTGAGCCCGTGACA | |
| | GTCTCCTGGAACTCTGGCGCCCTGACCTCCGGAGTGCACACA | |
| | TTCCCTGCTGTGCTGCAGTCCAGCGGCCTGTACTCCCTGTCTT | |
| | CCGTGGTGACCGTGCCAAGCTCTTCCCTGGGCACCCAGACAT | |
| | ATATCTGCAACGTGAATCACAAGCCTTCCAATACAAAGGTG | |
| | GACAAGAGGGTGGAGCCAAAGAGCTGTGATAAGACCCATAC | |
| | ATGCCCACCTTGTCCAGCTCCAGAGCTGCTGGGCGGCCCATC | |
| | CGTGTTCCTGTTTCCACCCAAGCCCAAGGACACCCTGATGAT | |
| | CTCTAGAACCCCAGAGGTGACATGCGTGGTGGTGGACGTGT | |
| | CCCACGAGGATCCCGAGGTGAAGTTTAACTGGTACGTGGAT | |
| | GGCGTGGAGGTGCATAATGCTAAGACAAAGCCCAGGGAGG | |
| | AGCAGTACAACAGCACCTATCGGGTGGTGTCTGTGCTGACA | |
| | GTGCTGCATCAGGACTGGCTGAACGGCAAGGAGTATAAGTG | |
| | CAAGGTGAGCAATAAGGCCCTGCCTGCTCCAATCGAGAAGA | |
| | CCATCTCTAAGGCCAAGGGCCAGCCCAGAGAGCCTCAGGTG | |
| | TACACACTGCCTCCAAGCCGCGAGGAGATGACCAAGAACCA | |
| | GGTGTCTCTGACATGTCTGGTGAAGGGCTTCTATCCCTCTGA | |
| | CATCGCTGTGGAGTGGGAGTCCAATGGCCAGCCTGAGAACA | |
| | ATTACAAGACCACACCCCCTGTGCTGGACTCCGATGGCAGCT | |
| | TCTTTCTGTATTCCAAGCTGACCGTGGATAAGAGCAGGTGGC | |
| | AGCAGGGCAACGTGTTCTCCTGTTCTGTGCTGCACGAAGCCC | |
| | TGCACTCCCATTATACTCAGAAGTCCCTGTCCCTGTCCCCTG | |
| | GAAAA | |
| 110 | GAGATCGTGATGACACAGTCTCCAGCCACCCTGTCCCTGTCC | FNI9-v8.1 light |
| | AGCGGAGAGAGAGCCACACTGAGCTGCAGGGCTAGCAGGTC | chain nucleotide |
| | CGTGTCCTCCAACCTGGCCTGGTACCAGCAGAAGCCTGGCC | |
| | AGGCTCCACGCCTGCTGATCTATGACGCCAGCACAAGAGCT | |
| | ACCGGCTTCTCTGCCAGGTTTGCTGGATCCGGAAGCGGAACC | |
| | GAGTTCACCCTGACAATCAGCTCTCTGCAGAGCGAGGACTCT | |
| | GCTATCTACTATTGCCAGCAGTACAACAATTGGCCCCCTTGG | |
| | ACATTCGGCCAGGGCACCAAGGTGGAGATCAAGCGTACGGT | |
| | GGCTGCACCATCTGTCTTCATCTTCCCGCCATCTGATGAGCA | |
| | ATTGAAATCTGGAACTGCCTCCGTGGTGTGCCTGCTGAACAA | |
| | TTTCTACCCCAGGGAGGCCAAGGTGCAGTGGAAGGTGGACA | |
| | ACGCTCTGCAGAGCGGCAATTCTCAGGAGTCCGTGACCGAG | |
| | CAGGACAGCAAGGATTCTACATATTCCCTGTCCAGCACCCTG | |
| | ACACTGAGCAAGGCCGATTACGAGAAGCACAAGGTGTATGC | |
| | TTGTGAGGTGACCCATCAGGGCCTGTCTTCCCCTGTGACAAA | |
| | GTCTTTCAACAGGGGAGAGTGT | |
| 111 | QVQLQQSGPGLVKPSQTLSLTCAISGDSVSSYNAVWNWIRQSP | FM08_MLNS |
| | SRGLEWLGRTYYRSGWYNDYAESVKSRITINPDTSKNQFSLQL | heavy chain |
| | NSVTPEDTAVYYCARSGHITVFGVNVDAFDMWGQGTMVTVSS | amino acid |
| | ASTKGPSVFPLAPSSKSTSGGTAALGCLVKDYFPEPVTVSWNSG | |
| | ALTSGVHTFPAVLQSSGLYSLSSVVTVPSSSLGTQTYICNVNHK | |
| | PSNTKVDKRVEPKSCDKTHTCPPCPAPELLGGPSVFLFPPKPKD | |
| | TLMISRTPEVTCVVVDVSHEDPEVKFNWYVDGVEVHNAKTKP | |
| | REEQYNSTYRVVSVLTVLHQDWLNGKEYKCKVSNKALPAPIE | |
| | KTISKAKGQPREPQVYTLPPSREEMTKNQVSLTCLVKGFYPSDI | |
| | AVEWESNGQPENNYKTTPPVLDSDGSFFLYSKLTVDKSRWQQ | |
| | GNVFSCSVLHEALHSHYTQKSLSLSPGK | |
| 112 | DIQMTQSPSSLSASVGDRVTITCRTSQSLSSYTHWYQQKPGKAP | FM08 light chain |
| | KLLIYAASSRGSGVPSRFSGSGSGTDFTLTISSLQPEDFATYYCQ | amino acid |
| | QSRTFGQGTKVEIKRTVAAPSVFIFPPSDEQLKSGTASVVCLLN | |
| | NFYPREAKVQWKVDNALQSGNSQESVTEQDSKDSTYSLSSTLT | |
| | LSKADYEKHKVYACEVTHQGLSSPVTKSFNRGEC | |
| 113 | AGCRAAAGCAGG | Primer Uni12 |
| 114 | caattggctctgtctctct | Forward Primer |
| 115 | atgaaattgatgttcgccc | Reverse Primer |
| 116 | gaagagccgatactagaa | Primer |
| 117 | ttctagtatcggctcttc | Primer |
| 118 | ACGCGTGATCAGCAAAAGCAGG | MBTuni-12 |
| | | Primer |
| 119 | ACGCGTGATCAGTAGAAACAAGG | MBTuni-13 |
| | | Primer |
| 120 | ATTCTCCTTGGAATTTGCCCTT | Forward Primer |
| 121 | CATAGCGTAAAAGGAGCAACA | Reverse Primer |

TABLE 2
SEQ ID NOS. (Amino Acid Sequences) of Certain FNI9 Antibodies

| | | | CDRH2 + | CDRH | | | | | |
|---|---|---|---|---|---|---|---|---|---|
| | CDRH1 | CDRH2 | flanking | 3 | VH | CDRL1 | CDRL2 | CDRL3 | VL |
| FNI9 | 3 | 4 | — | 5 | 2 | 9 | 10 | 11 | 8 |
| FNI9- | 3 | 4 | — | 15 | 14 | | | | |
| VH- | | | | | | | | | |
| W110F | | | | | | | | | |
| FNI9- | | | — | | | 9 | 10 | 18 | 17 |
| VK- | | | | | | | | | |
| W94F | | | | | | | | | |
| FNI9- | | | — | | | 9 | 10 | 21 | 20 |
| VK- | | | | | | | | | |
| W97F | | | | | | | | | |
| FNI9- | | | — | | | 9 | 10 | 24 | 23 |
| VK- | | | | | | | | | |
| W94F- | | | | | | | | | |
| W97F | | | | | | | | | |
| FNI9- | | | — | | | 32 | 96 | 33 | 31 |
| v5-VK | | | | | | | | | |
| FNI9- | | | — | | | 9 | 10 | 11 | 37 |
| variant | | | | | | | | | |
| VL | | | | | | | | | |
| FNI9- | 3 | 4 | — | 5 | 45 | | | | |
| VH- | | | | | | | | | |
| FR124 | | | | | | | | | |
| GL | | | | | | | | | |
| FNI9- | 47 | 4 | — | 5 | 46 | | | | |
| VH.4 | | | | | | | | | |
| FNI9- | 49 | 4 | — | 5 | 48 | | | | |
| VH.5 | | | | | | | | | |
| FNI9- | 3 | 4 | — | 51 | 50 | | | | |
| VH.6 | | | | | | | | | |
| FNI9- | 3 | 4 | — | 53 | 52 | | | | |
| VH.7 | | | | | | | | | |
| FNI9- | 55 | 4 | — | 5 | 54 | | | | |
| VH.8 | | | | | | | | | |
| FNI9- | 3 | 57 | — | 5 | 56 | | | | |
| VH.9 | | | | | | | | | |
| FNI9- | 3 | 4 | 59 | 5 | 58 | | | | |
| VH.10 | | | | | | | | | |
| FNI9- | 3 | 61 | 62 | 5 | 60 | | | | |
| VH.11 | | | | | | | | | |
| FNI9- | 3 | 61 | 64 | 5 | 63 | | | | |
| VH.12 | | | | | | | | | |
| FNI9- | 3 | 4 | | 5 | 65 | | | | |
| VH.13 | | | | | | | | | |
| FNI9- | | | | | | 9 | 10 | 67 | 66 |
| VK.7 | | | | | | | | | |
| FNI9- | | | | | | 9 | 10 | 11 | 68 |
| VK.8 | | | | | | | | | |
| FNI9- | 3 | 4 | | 5 | 65 | 9 | 10 | 11 | 68 |
| v13.8 | | | | | | | | | |
| FNI9- | 47 | 4 | — | 5 | 46 | 9 | 10 | 11 | 8 |
| v4.1 | | | | | | | | | |
| FNI9- | 49 | 4 | — | 5 | 48 | 9 | 10 | 11 | 8 |
| v5.1 | | | | | | | | | |
| FNI9- | 3 | 4 | — | 51 | 50 | 9 | 10 | 11 | 8 |
| v6.1 | | | | | | | | | |
| FNI9- | 3 | 4 | — | 53 | 52 | 9 | 10 | 11 | 8 |
| v7.1 | | | | | | | | | |
| FNI9- | 55 | 4 | — | 5 | 54 | 9 | 10 | 11 | 8 |
| v8.1 | | | | | | | | | |
| FNI9- | 3 | 57 | — | 5 | 56 | 9 | 10 | 11 | 8 |
| v9.1 | | | | | | | | | |
| FNI9- | 3 | 4 | 59 | 5 | 58 | 9 | 10 | 11 | 8 |
| v10.1 | | | | | | | | | |
| FNI9- | 3 | 61 | 62 | 5 | 60 | 9 | 10 | 11 | 8 |
| v11.1 | | | | | | | | | |
| FNI9- | 3 | 61 | 64 | 5 | 63 | 9 | 10 | 11 | 8 |
| v12.1 | | | | | | | | | |
| FNI9- | 47 | 4 | — | 5 | 46 | 9 | 10 | 67 | 66 |
| v4.7 | | | | | | | | | |
| FNI9- | 49 | 4 | — | 5 | 48 | 9 | 10 | 67 | 66 |
| v5.7 | | | | | | | | | |
| FNI9- | 3 | 4 | — | 51 | 50 | 9 | 10 | 67 | 66 |
| v6.7 | | | | | | | | | |
| FNI9- | 3 | 4 | — | 53 | 52 | 9 | 10 | 67 | 66 |
| v7.7 | | | | | | | | | |
| FNI9- | 55 | 4 | — | 5 | 54 | 9 | 10 | 67 | 66 |
| v8.7 | | | | | | | | | |
| FNI9- | 3 | 57 | — | 5 | 56 | 9 | 10 | 67 | 66 |
| v9.7 | | | | | | | | | |
| FNI9- | 3 | 4 | 59 | 5 | 58 | 9 | 10 | 67 | 66 |
| v10.7 | | | | | | | | | |
| FNI9- | 3 | 61 | 62 | 5 | 60 | 9 | 10 | 67 | 66 |
| v11.7 | | | | | | | | | |
| FNI9- | 3 | 61 | 64 | 5 | 63 | 9 | 10 | 67 | 66 |
| v12.7 | | | | | | | | | |
| FNI9- | 3 | 4 | — | 5 | 2 | 9 | 10 | 11 | 8 |
| v1.1 | | | | | | | | | |

TABLE 3
Neuraminidase Amino Acid Position Comparison (H1N1
California.07.2009 to H3N2 New York.392.2004)

| residue_N1 | position_N1 | residue_N2 | position_N2 |
|---|---|---|---|
| M | 1 | M | 1 |
| N | 2 | N | 2 |
| P | 3 | P | 3 |
| N | 4 | N | 4 |
| Q | 5 | Q | 5 |
| K | 6 | K | 6 |
| I | 7 | I | 7 |
| I | 8 | I | 8 |
| T | 9 | T | 9 |
| I | 10 | I | 10 |
| G | 11 | G | 11 |
| S | 12 | S | 12 |
| V | 13 | V | 13 |
| C | 14 | S | 14 |
| M | 15 | L | 15 |
| T | 16 | T | 16 |
| I | 17 | I | 17 |
| G | 18 | S | 18 |
| M | 19 | T | 19 |
| A | 20 | I | 20 |
| N | 21 | C | 21 |
| L | 22 | F | 22 |
| I | 23 | F | 23 |
| L | 24 | M | 24 |
| Q | 25 | Q | 25 |
| I | 26 | I | 26 |
| G | 27 | A | 27 |
| N | 28 | I | 28 |
| I | 29 | L | 29 |
| I | 30 | I | 30 |
| S | 31 | T | 31 |
| I | 32 | T | 32 |
| W | 33 | V | 33 |
| I | 34 | T | 34 |
| S | 35 | L | 35 |
| H | 36 | H | 36 |
| S | 37 | F | 37 |
| I | 38 | K | 38 |
| Q | 39 | Q | 39 |
| L | 40 | Y | 40 |
| G | 41 | E | 41 |
| N | 42 | F | 42 |
| Q | 43 | N | 43 |
| N | 44 | S | 44 |
| Q | 45 | P | 45 |
| I | 46 | P | 46 |
| E | 47 | N | 47 |
| T | 48 | — | NA |
| C | 49 | — | NA |
| N | 50 | N | 48 |
| Q | 51 | Q | 49 |
| S | 52 | V | 50 |
| V | 53 | M | 51 |
| I | 54 | L | 52 |
| T | 55 | C | 53 |
| Y | 56 | E | 54 |
| E | 57 | P | 55 |
| N | 58 | T | 56 |
| N | 59 | I | 57 |
| T | 60 | I | 58 |
| W | 61 | E | 59 |
| V | 62 | R | 60 |
| N | 63 | N | 61 |
| Q | 64 | I | 62 |
| T | 65 | T | 63 |
| — | NA | E | 64 |
| Y | 66 | I | 65 |
| V | 67 | V | 66 |
| N | 68 | Y | 67 |
| I | 69 | L | 68 |
| S | 70 | T | 69 |
| N | 71 | N | 70 |
| T | 72 | T | 71 |
| N | 73 | T | 72 |
| F | 74 | I | 73 |
| A | 75 | E | 74 |
| A | 76 | K | 75 |
| G | 77 | E | 76 |
| Q | 78 | M | 77 |
| S | 79 | C | 78 |
| V | 80 | P | 79 |
| V | 81 | K | 80 |
| S | 82 | L | 81 |
| V | 83 | A | 82 |
| K | 84 | E | 83 |
| L | 85 | Y | 84 |
| A | 86 | R | 85 |
| G | 87 | N | 86 |
| N | 88 | W | 87 |
| S | 89 | S | 88 |
| S | 90 | K | 89 |
| — | NA | P | 90 |
| L | 91 | Q | 91 |
| C | 92 | C | 92 |
| P | 93 | D | 93 |
| V | 94 | I | 94 |
| S | 95 | T | 95 |
| G | 96 | G | 96 |
| W | 97 | F | 97 |
| A | 98 | A | 98 |
| I | 99 | P | 99 |
| Y | 100 | F | 100 |
| S | 101 | S | 101 |
| K | 102 | K | 102 |
| D | 103 | D | 103 |
| N | 104 | N | 104 |
| S | 105 | S | 105 |
| V | 106 | I | 106 |
| R | 107 | R | 107 |
| I | 108 | L | 108 |
| G | 109 | S | 109 |
| S | 110 | A | 110 |
| K | 111 | G | 111 |
| G | 112 | G | 112 |
| D | 113 | D | 113 |
| V | 114 | I | 114 |
| F | 115 | W | 115 |
| V | 116 | V | 116 |
| I | 117 | T | 117 |
| R | 118 | R | 118 |
| E | 119 | E | 119 |
| P | 120 | P | 120 |
| F | 121 | Y | 121 |
| I | 122 | V | 122 |
| S | 123 | S | 123 |
| C | 124 | C | 124 |
| S | 125 | D | 125 |
| P | 126 | P | 126 |
| L | 127 | D | 127 |
| E | 128 | K | 128 |
| C | 129 | C | 129 |
| R | 130 | Y | 130 |
| T | 131 | Q | 131 |
| F | 132 | F | 132 |
| F | 133 | A | 133 |
| L | 134 | L | 134 |
| T | 135 | G | 135 |
| Q | 136 | Q | 136 |
| G | 137 | G | 137 |
| A | 138 | T | 138 |
| L | 139 | T | 139 |
| L | 140 | L | 140 |
| N | 141 | N | 141 |
| D | 142 | N | 142 |
| K | 143 | V | 143 |
| H | 144 | H | 144 |
| S | 145 | S | 145 |
| N | 146 | N | 146 |
| G | 147 | D | 147 |
| T | 148 | T | 148 |
| I | 149 | V | 149 |
| K | 150 | H | 150 |
| D | 151 | D | 151 |
| R | 152 | R | 152 |
| S | 153 | T | 153 |
| P | 154 | P | 154 |
| Y | 155 | Y | 155 |
| R | 156 | R | 156 |
| T | 157 | T | 157 |
| L | 158 | L | 158 |
| M | 159 | L | 159 |
| S | 160 | M | 160 |
| C | 161 | N | 161 |
| P | 162 | E | 162 |
| I | 163 | L | 163 |
| G | 164 | G | 164 |
| E | 165 | — | NA |
| V | 166 | V | 165 |
| P | 167 | P | 166 |
| S | 168 | F | 167 |
| P | 169 | H | 168 |
| Y | 170 | L | 169 |
| N | 171 | G | 170 |
| S | 172 | T | 171 |
| R | 173 | K | 172 |
| F | 174 | Q | 173 |
| E | 175 | V | 174 |
| S | 176 | C | 175 |
| V | 177 | I | 176 |
| A | 178 | A | 177 |
| W | 179 | W | 178 |
| S | 180 | S | 179 |
| A | 181 | S | 180 |
| S | 182 | S | 181 |
| A | 183 | S | 182 |
| C | 184 | C | 183 |
| H | 185 | H | 184 |
| D | 186 | D | 185 |
| G | 187 | G | 186 |
| I | 188 | K | 187 |
| N | 189 | A | 188 |
| W | 190 | W | 189 |
| L | 191 | L | 190 |
| T | 192 | H | 191 |
| I | 193 | V | 192 |
| G | 194 | C | 193 |
| I | 195 | V | 194 |
| S | 196 | T | 195 |
| G | 197 | G | 196 |
| P | 198 | D | 197 |
| D | 199 | D | 198 |
| N | 200 | K | 199 |
| G | 201 | N | 200 |
| A | 202 | A | 201 |
| V | 203 | T | 202 |
| A | 204 | A | 203 |
| V | 205 | S | 204 |
| L | 206 | F | 205 |
| K | 207 | I | 206 |
| Y | 208 | Y | 207 |
| N | 209 | N | 208 |
| G | 210 | G | 209 |
| I | 211 | R | 210 |
| I | 212 | L | 211 |
| T | 213 | V | 212 |
| D | 214 | D | 213 |
| T | 215 | S | 214 |
| I | 216 | I | 215 |
| K | 217 | V | 216 |
| S | 218 | S | 217 |
| W | 219 | W | 218 |
| R | 220 | S | 219 |
| N | 221 | K | 220 |
| N | 222 | K | 221 |
| I | 223 | I | 222 |
| L | 224 | L | 223 |
| R | 225 | R | 224 |
| T | 226 | T | 225 |
| Q | 227 | Q | 226 |
| E | 228 | E | 227 |
| S | 229 | S | 228 |
| E | 230 | E | 229 |
| C | 231 | C | 230 |
| A | 232 | V | 231 |
| C | 233 | C | 232 |
| V | 234 | I | 233 |
| N | 235 | N | 234 |
| G | 236 | G | 235 |
| S | 237 | T | 236 |
| C | 238 | C | 237 |
| F | 239 | T | 238 |
| T | 240 | V | 239 |
| V | 241 | V | 240 |
| M | 242 | M | 241 |
| T | 243 | T | 242 |
| D | 244 | D | 243 |
| G | 245 | G | 244 |
| P | 246 | S | 245 |
| S | 247 | A | 246 |
| N | 248 | S | 247 |
| G | 249 | G | 248 |
| Q | 250 | K | 249 |
| A | 251 | A | 250 |
| S | 252 | D | 251 |
| Y | 253 | T | 252 |
| K | 254 | K | 253 |
| I | 255 | I | 254 |
| F | 256 | L | 255 |
| R | 257 | F | 256 |
| I | 258 | I | 257 |
| E | 259 | E | 258 |
| K | 260 | E | 259 |
| G | 261 | G | 260 |
| K | 262 | K | 261 |
| I | 263 | I | 262 |
| V | 264 | I | 263 |
| K | 265 | H | 264 |
| S | 266 | T | 265 |
| V | 267 | S | 266 |
| E | 268 | T | 267 |
| M | 269 | L | 268 |
| N | 270 | S | 269 |
| A | 271 | G | 270 |
| P | 272 | S | 271 |
| N | 273 | A | 272 |
| Y | 274 | Q | 273 |
| H | 275 | H | 274 |
| Y | 276 | V | 275 |
| E | 277 | E | 276 |
| E | 278 | E | 277 |
| C | 279 | C | 278 |
| S | 280 | S | 279 |
| C | 281 | C | 280 |
| Y | 282 | Y | 281 |
| P | 283 | P | 282 |
| D | 284 | R | 283 |
| S | 285 | Y | 284 |
| S | 286 | P | 285 |
| E | 287 | G | 286 |
| I | 288 | V | 287 |
| T | 289 | R | 288 |
| C | 290 | C | 289 |
| V | 291 | V | 290 |
| C | 292 | C | 291 |
| R | 293 | R | 292 |
| D | 294 | D | 293 |
| N | 295 | N | 294 |
| W | 296 | W | 295 |
| H | 297 | K | 296 |
| G | 298 | G | 297 |
| S | 299 | S | 298 |
| N | 300 | N | 299 |
| R | 301 | R | 300 |
| P | 302 | P | 301 |
| W | 303 | I | 302 |
| V | 304 | V | 303 |
| S | 305 | D | 304 |
| F | 306 | I | 305 |
| N | 307 | N | 306 |
| — | NA | I | 307 |
| Q | 308 | K | 308 |
| N | 309 | D | 309 |
| L | 310 | Y | 310 |
| E | 311 | S | 311 |
| Y | 312 | I | 312 |
| Q | 313 | V | 313 |
| I | 314 | S | 314 |
| G | 315 | S | 315 |
| Y | 316 | Y | 316 |
| I | 317 | V | 317 |
| C | 318 | C | 318 |
| S | 319 | S | 319 |
| G | 320 | G | 320 |
| I | 321 | L | 321 |
| F | 322 | V | 322 |
| G | 323 | G | 323 |
| D | 324 | D | 324 |
| N | 325 | T | 325 |
| P | 326 | P | 326 |
| R | 327 | R | 327 |
| P | 328 | K | 328 |
| N | 329 | N | 329 |
| D | 330 | D | 330 |
| K | 331 | S | 331 |
| T | 332 | S | 332 |
| G | 333 | S | 333 |
| S | 334 | S | 334 |
| C | 335 | S | 335 |
| — | NA | H | 336 |
| — | NA | C | 337 |
| — | NA | L | 338 |
| G | 336 | D | 339 |
| P | 337 | P | 340 |
| V | 338 | N | 341 |
| S | 339 | N | 342 |
| S | 340 | E | 343 |
| N | 341 | E | 344 |
| G | 342 | G | 345 |
| A | 343 | G | 346 |
| N | 344 | H | 347 |
| G | 345 | G | 348 |
| V | 346 | V | 349 |
| K | 347 | K | 350 |
| G | 348 | G | 351 |
| F | 349 | W | 352 |
| S | 350 | A | 353 |
| F | 351 | F | 354 |
| K | 352 | D | 355 |
| Y | 353 | D | 356 |
| G | 354 | G | 357 |
| N | 355 | N | 358 |
| G | 356 | D | 359 |
| V | 357 | V | 360 |
| W | 358 | W | 361 |
| I | 359 | M | 362 |
| G | 360 | G | 363 |
| R | 361 | R | 364 |
| T | 362 | T | 365 |
| K | 363 | I | 366 |
| S | 364 | S | 367 |
| I | 365 | E | 368 |
| S | 366 | K | 369 |
| S | 367 | L | 370 |
| R | 368 | R | 371 |
| N | 369 | S | 372 |
| G | 370 | G | 373 |
| F | 371 | Y | 374 |
| E | 372 | E | 375 |
| M | 373 | T | 376 |
| I | 374 | F | 377 |
| W | 375 | K | 378 |
| D | 376 | V | 379 |
| P | 377 | I | 380 |
| N | 378 | E | 381 |
| G | 379 | G | 382 |
| W | 380 | W | 383 |
| T | 381 | S | 384 |
| G | 382 | K | 385 |
| T | 383 | P | 386 |
| D | 384 | N | 387 |
| N | 385 | S | 388 |
| N | 386 | K | 389 |
| F | 387 | L | 390 |
| S | 388 | Q | 391 |
| I | 389 | I | 392 |
| — | NA | N | 393 |
| K | 390 | R | 394 |
| Q | 391 | Q | 395 |
| D | 392 | V | 396 |
| I | 393 | I | 397 |
| V | 394 | V | 398 |
| G | 395 | D | 399 |
| I | 396 | R | 400 |
| N | 397 | G | 401 |
| E | 398 | N | 402 |
| W | 399 | R | 403 |
| S | 400 | S | 404 |
| G | 401 | G | 405 |
| Y | 402 | Y | 406 |
| S | 403 | S | 407 |
| G | 404 | G | 408 |
| S | 405 | I | 409 |
| F | 406 | F | 410 |
| V | 407 | — | NA |
| Q | 408 | — | NA |
| H | 409 | — | NA |
| P | 410 | — | NA |
| E | 411 | S | 411 |
| L | 412 | V | 412 |
| T | 413 | E | 413 |
| G | 414 | G | 414 |
| L | 415 | K | 415 |
| D | 416 | S | 416 |
| C | 417 | C | 417 |
| I | 418 | I | 418 |
| R | 419 | N | 419 |
| P | 420 | R | 420 |
| C | 421 | C | 421 |
| F | 422 | F | 422 |
| W | 423 | Y | 423 |
| V | 424 | V | 424 |
| E | 425 | E | 425 |
| L | 426 | L | 426 |
| I | 427 | I | 427 |
| R | 428 | R | 428 |
| G | 429 | G | 429 |
| R | 430 | R | 430 |
| P | 431 | K | 431 |
| K | 432 | E | 432 |
| E | 433 | E | 433 |
| N | 434 | T | 434 |
| T | 435 | E | 435 |
| I | 436 | V | 436 |
| — | NA | L | 437 |
| W | 437 | W | 438 |
| T | 438 | T | 439 |
| S | 439 | S | 440 |
| G | 440 | N | 441 |
| S | 441 | S | 442 |
| S | 442 | I | 443 |
| I | 443 | V | 444 |
| S | 444 | V | 445 |
| F | 445 | F | 446 |
| C | 446 | C | 447 |
| G | 447 | G | 448 |
| V | 448 | T | 449 |
| N | 449 | S | 450 |
| S | 450 | G | 451 |
| D | 451 | T | 452 |
| T | 452 | Y | 453 |
| V | 453 | G | 454 |
| G | 454 | T | 455 |
| W | 455 | G | 456 |
| S | 456 | S | 457 |
| W | 457 | W | 458 |
| P | 458 | P | 459 |
| D | 459 | D | 460 |
| G | 460 | G | 461 |
| A | 46 | A | 462 |
| E | 462 | D | 463 |
| L | 463 | I | 464 |
| P | 464 | N | 465 |
| F | 465 | L | 466 |
| T | 466 | — | NA |
| I | 467 | M | 467 |
| D | 468 | P | 468 |
| K | 469 | I | 469 |

### EXAMPLES

### Example 1

### Identification and Testing of Anti-NA Monoclonal Antibodies

Peripheral blood mononuclear cells (PBMCs) from anonymous human donors were selected based on binding of the corresponding serum against N1 and N4 (G1); and N2, N3 and N9 (G2) influenza pseudoviruses. Donors were selected by screening serum from tonsillar donor samples (n=50) for reactivity against neuraminidase subtype N1 and N2 antigens, and serum from PBMC donor samples (n=124) for reactivity against neuraminidase subtype N4, N3, and N9. Neuraminidase antigens for screening were expressed in mammalian cells and binding was evaluated by flow cytometry.

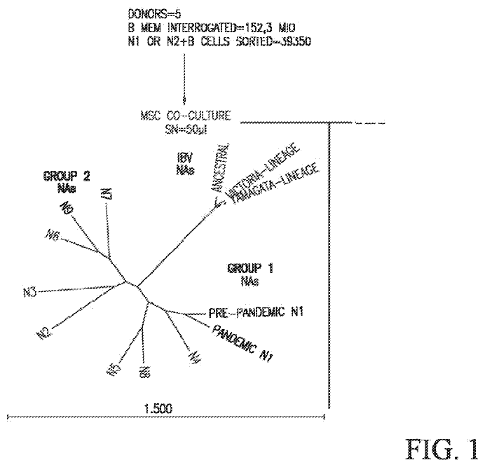

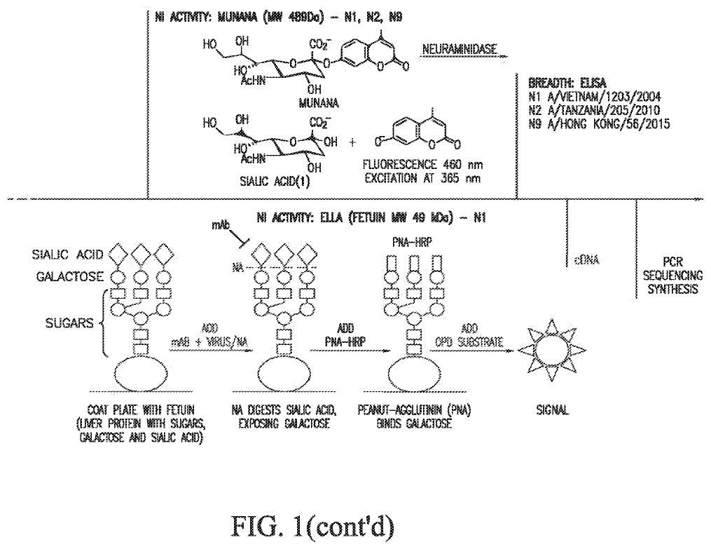

B memory cells from five donors were sorted by flow cytometry for input into the discovery workflow (FIG. 1). Single sorted B cells (n=39,350) were co-cultured with mesenchymal stromal cells (MSC) in 50 μl cultures to stimulate antibody secretion. Secreted antibodies were evaluated by binding and NA inhibition assays. Inhibition of N1 sialidase activity was evaluated using ELLA (enzyme-linked lectin assay), an absorbance-based assay that utilizes a large glycoprotein substrate, fetuin, as a substrate for sialic acid cleavage by NA (Lambre et al. J Immunol Methods. 1990). Inhibition of N1, N2, and N9 sialidase activity was measured using a fluorescence-based assay that measures cleavage of the 2′-(4-Methylumbelliferyl)-α-D-N-acetylneuraminic acid (MUNANA) by the NA enzyme (Potier et al. Anal. Biochem. 1979.).

Binding to NAs from group 1 IAV N1 A/Vietnam/1203/2004, and group 2 IAVs N2 A/Tanzania/205/2010 and N9 A/Hong Kong/56/2015 was evaluated by ELISA to determine breadth. Antibody sequences from selected B cells were cloned as cDNAs and sequenced.

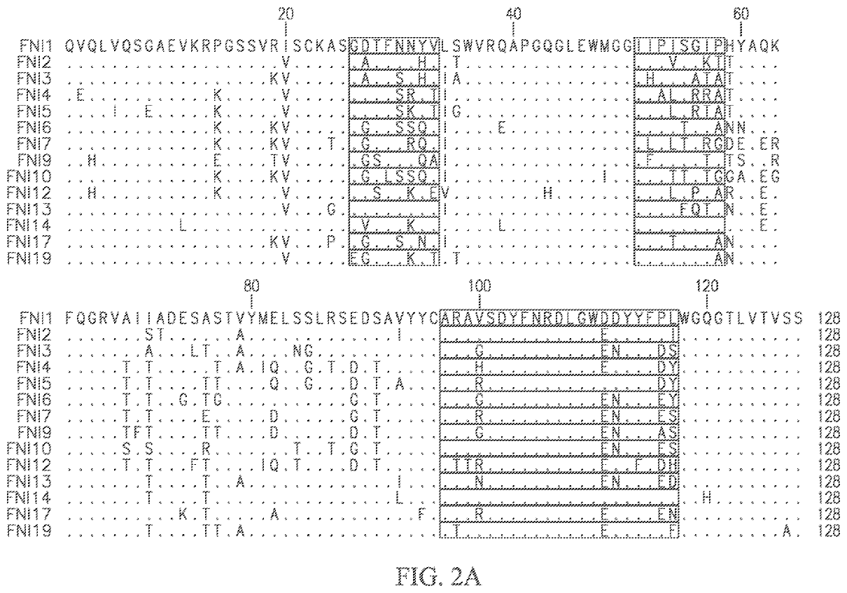

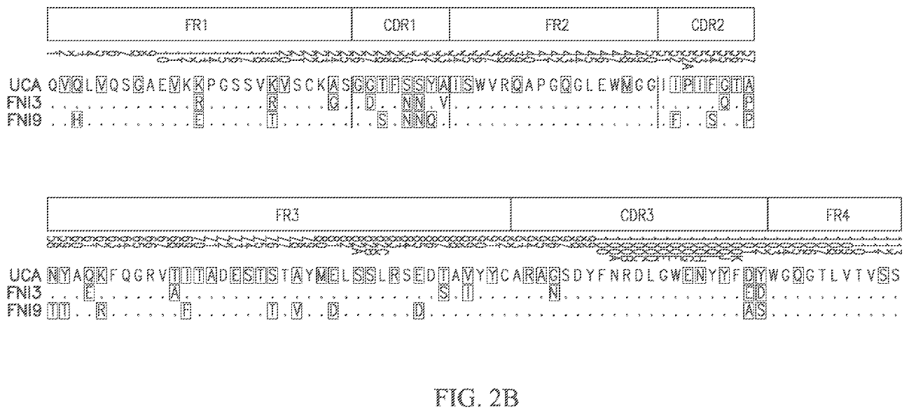

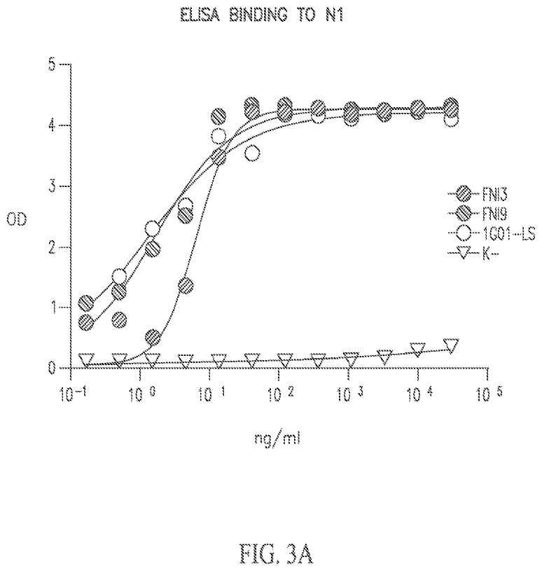

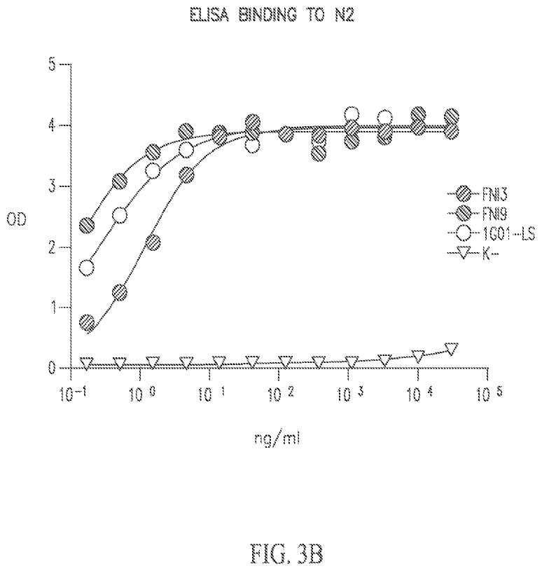

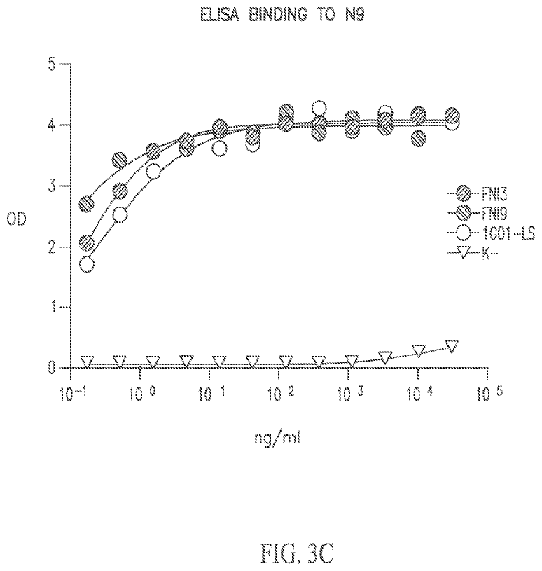

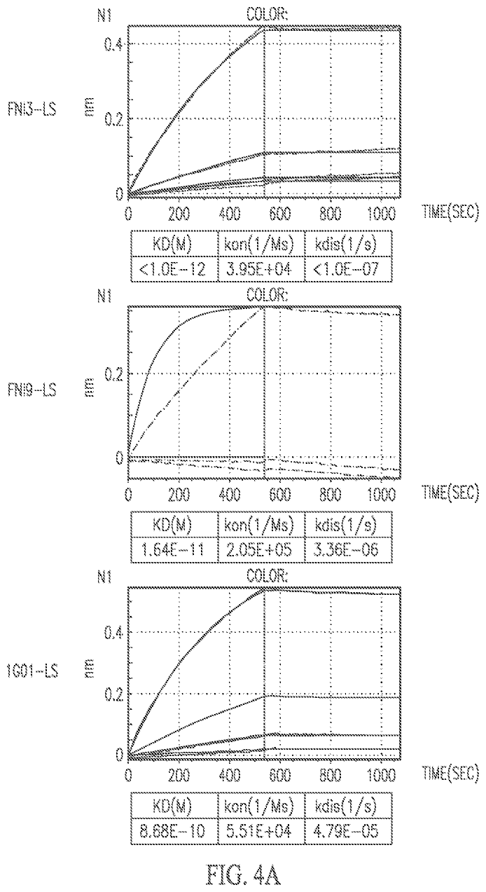

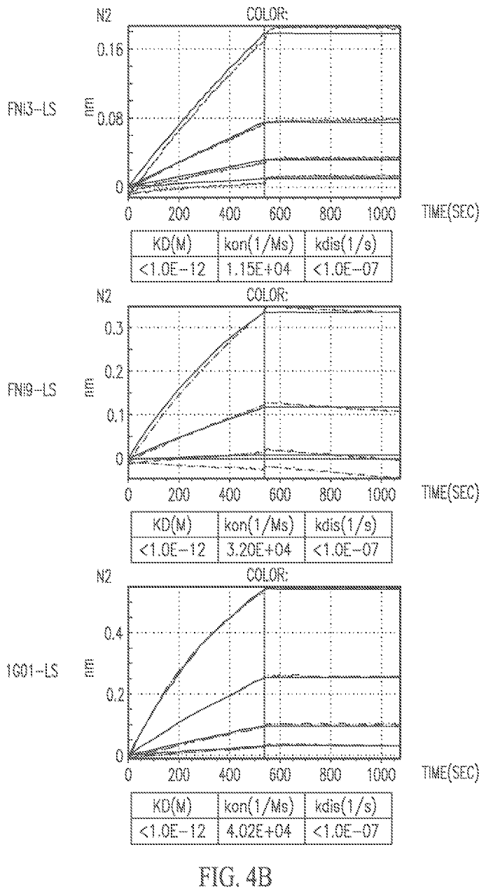

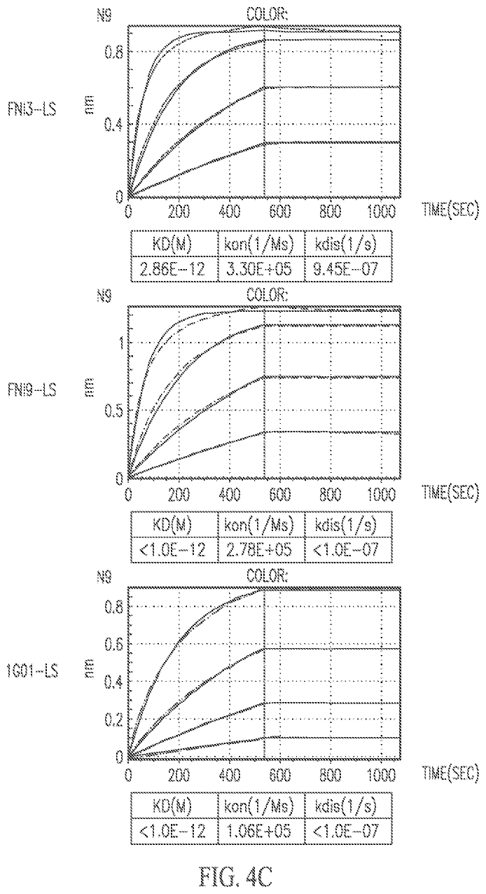

Fourteen clonally related monoclonal antibodies resulted from the discovery workflow (FIG. 2A). FNI3 and FNI9 (FNI9 comprises the following VH and VL amino acid sequences: VH: SEQ ID NO:2; VL: SEQ ID NO:8) were selected for further evaluation and testing. Alignment of FNI3 and FNI9 VH with that of the unmutated common ancestor, “UCA”, is shown in FIG. 2B. The UCA binds to a breadth of IAV and IBV NAs (data not shown). Binding of FNI3 and FNI9 to NA subtypes was evaluated. ELISA (enzyme-linked immunosorbent assay) was used to measure binding of FNI3 and FNI9 to N1 (FIG. 3A), N2 (FIG. 3B), and N9 (FIG. 3C) and reported as optical density (OD) versus concentration in ng/ml. Bio-Layer Interferometry (BLI) was used to measure KD, association (kon), and dissociation (kdis) of FNI3 and FNI9 for N1 binding (FIG. 4A), N2 (FIG. 4B), and N9 (FIG. 4C). Binding by a comparator antibody, 1G01-LS (1G01 is described by Stadlbauer et al. (Science 366(6464):499-504 (2019); see FIG. 1B; the VH and VL amino acid sequences of antibody 1G01, as well as those of antibodies 1E01 and 1G04, are incorporated herein by reference), and in these experiments bore M428L and N434S Fc mutations), was also measured by ELISA and BLI assays. A negative control antibody, K−, was used in the ELISA assays.

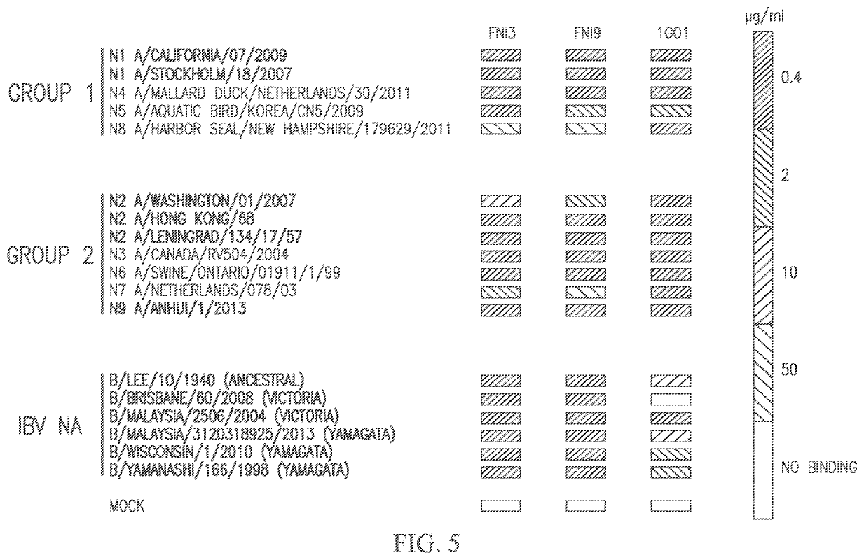

Binding of FNI3 and FNI9 to NAs from group I IAVs, group II IAVs, and IBVs is summarized in FIG. 5 (with comparator 1G01). Binding was quantified using a FACS-based assay in which NAs were expressed on the surface of mammalian cells. Briefly, Expi-CHO cells were transiently transfected with plasmids encoding different IAV and IBV NAs. At 48 hours post-transfection cells were incubated with the serial dilutions of the different mAbs. After 60 minutes incubation, the cells were washed and then incubated with an anti-Human IgG-AF647 secondary antibody. Cells were then washed twice and antibody binding was evaluated at the FACS. 1G01 was used as a comparator.

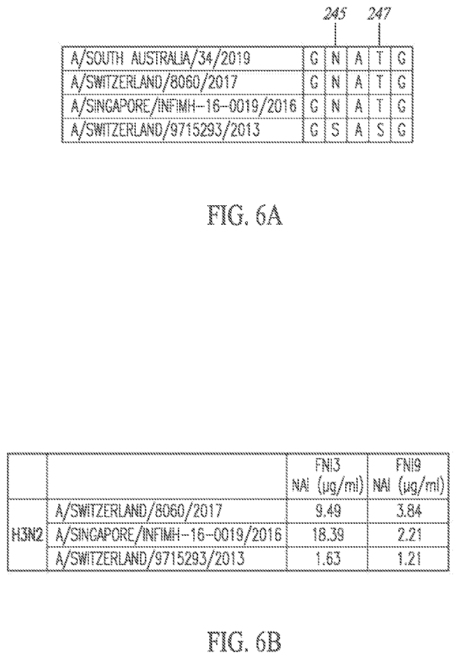

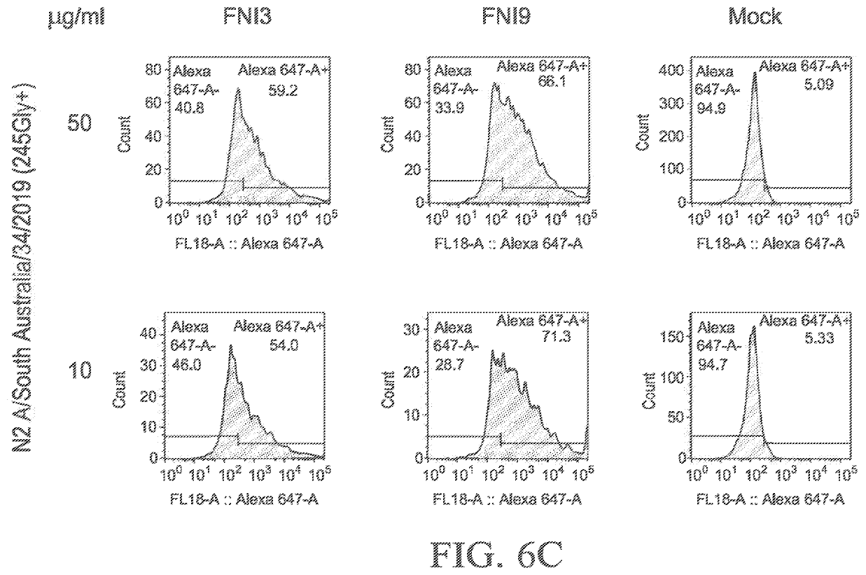

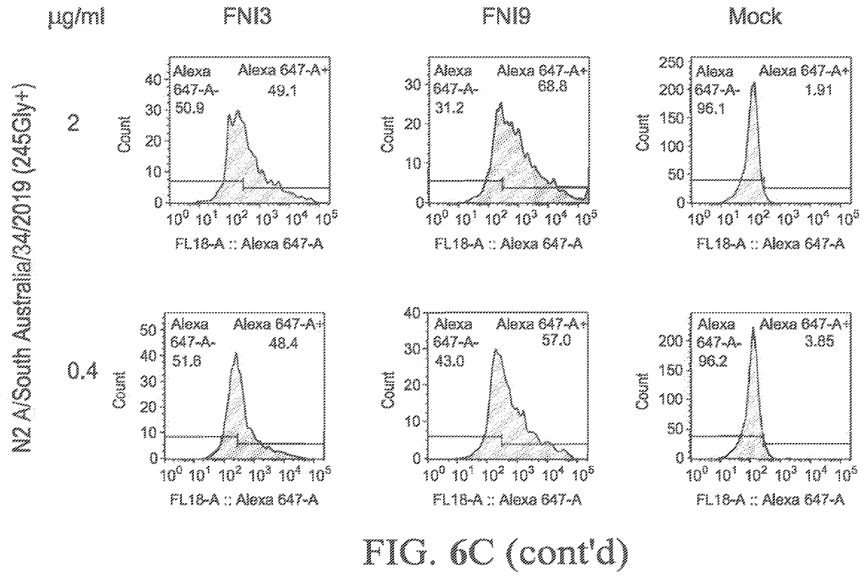

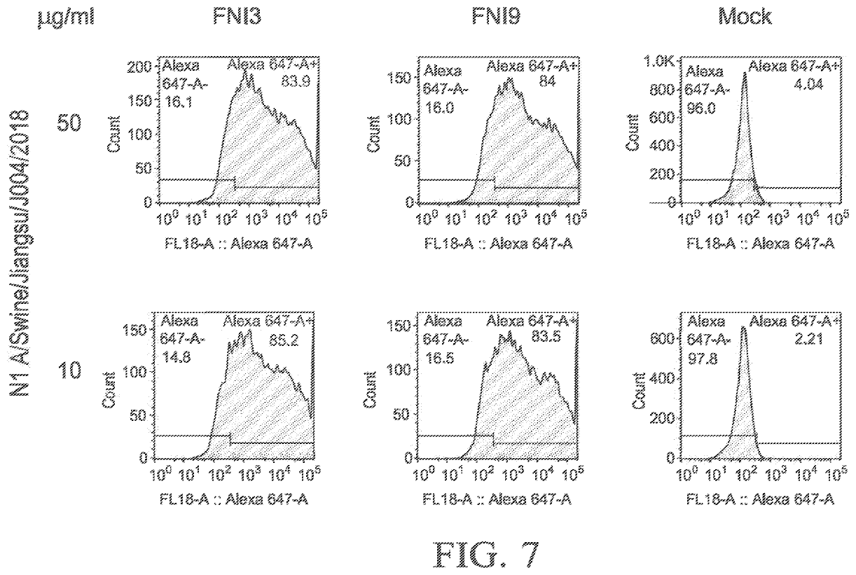

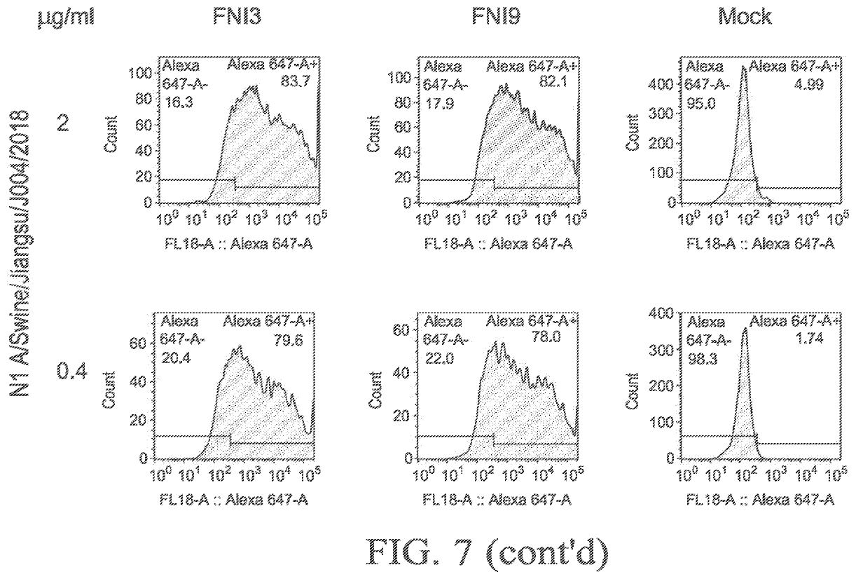

Glycosylation of influenza neuraminidase has implications for immune evasion and viral fitness in a host population. Glycosylation sites can occur at positions 245 (245Gly+) and 247 (247Gly+) (Wan et al. Nat Microbiology. 2019). Exemplary 245Gly+ and 247+Gly modification sites in A/South Australia/34/2019, A/Switzerland/8060/2017, A/Singapore/INFIMH-16-0019/2016, and A/Switzerland/9715293/2013 are shown in FIG. 6A. FIG. 6B shows inhibition of sialidase activity (NAI) activity against A/Switzerland/8060/2017, A/Singapore/INFIMH-16-0019/2016, and A/Switzerland/9715293/2013 live virus stocks, reported as EC50 in μg/ml. Binding of FNI3 and FNI9 to N2 in mammalian cells infected with A/South Australia/34/2019 (245Gly+) was measured by flow cytometry (FIG. 6C). Eurasian avian-like influenza virus strains isolated from swine are genetically diverse (Sun et al. Proc Natl Acad Sci USA. 2020). Binding of FNI3 and FNI9 to NA in mammalian cells infected with a H1N1 Swine Eurasian avian-like (EA) strain, A/Swine/Jiangsu/J004/2018 was measured by flow cytometry, as shown in FIG. 7.

The potential for polyreactivity of FNI3 and FNI9 was evaluated in human epithelial type 2 (HEP-2) cells. A comparator anti-HA antibody, FI6v3, was used as a positive control, and anti-paramyxovirus antibody “MPE8” (Corti et al. Nature 501(7467):439-43 (2013)) was included as a negative control. FNI3 and FNI9 showed a lack of polyreactivity in HEP-2 cells (data not shown).

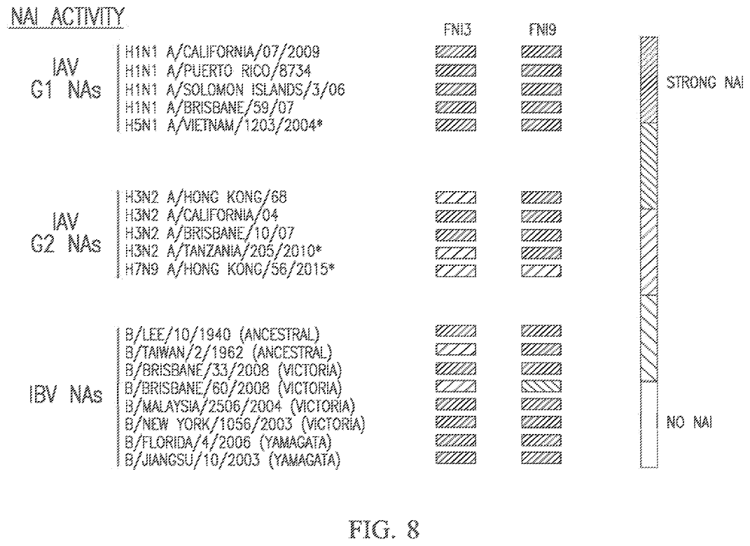

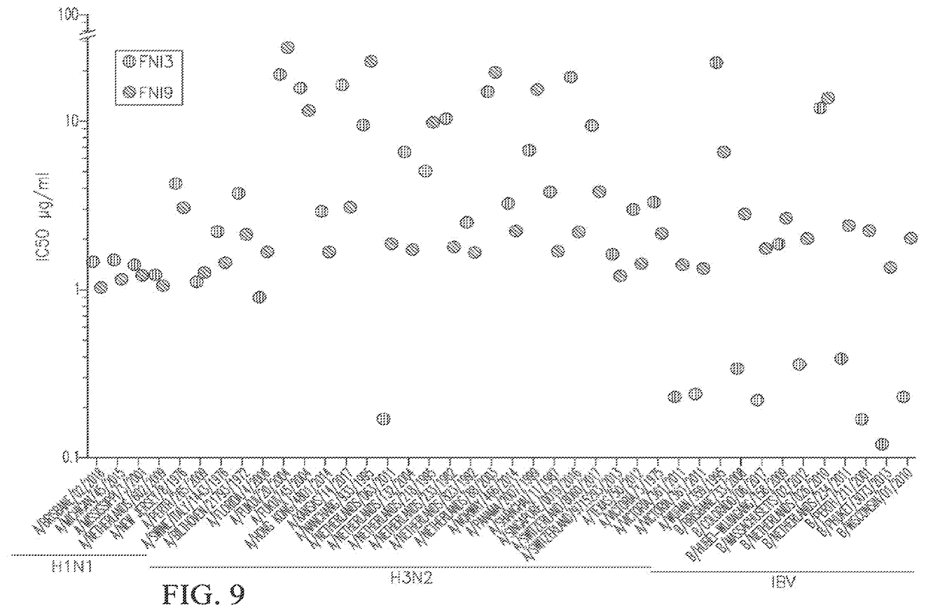

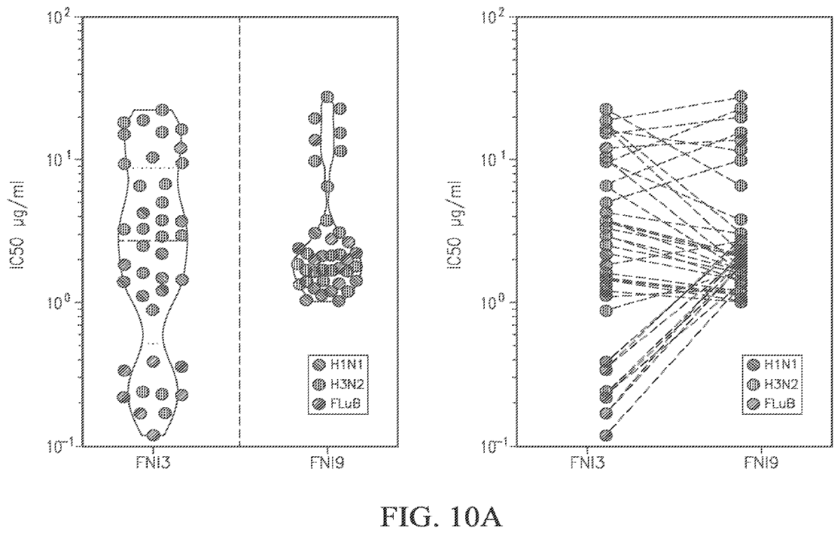

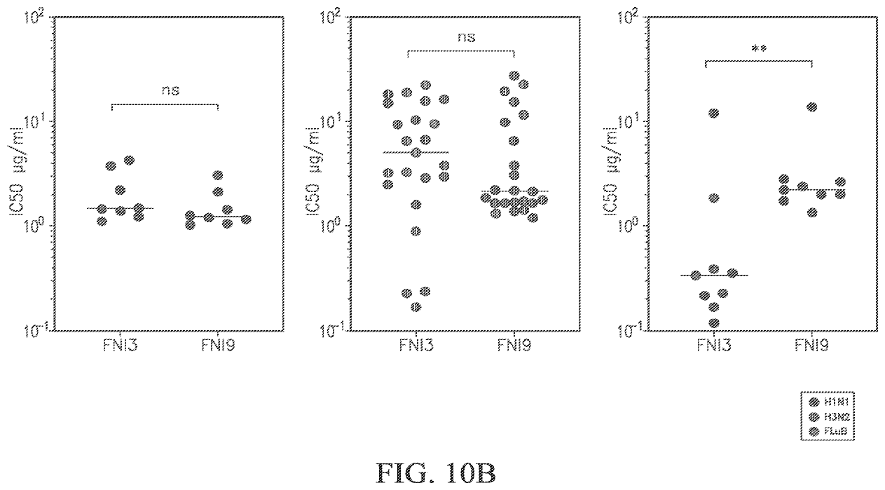

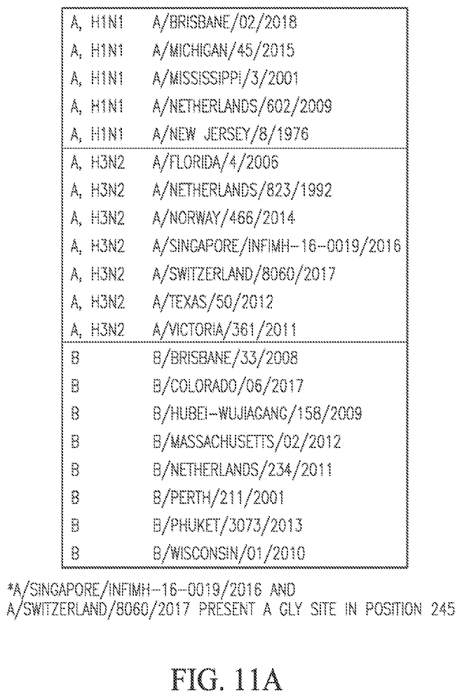

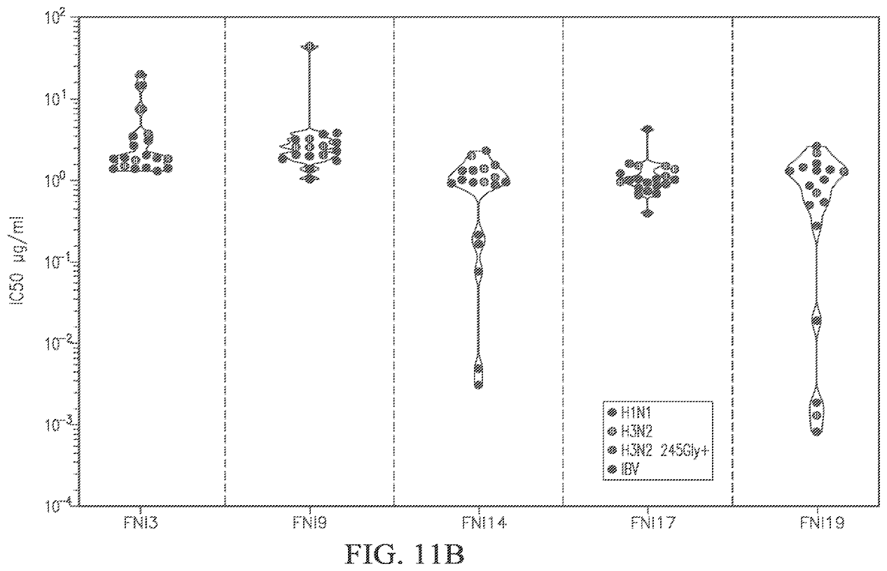

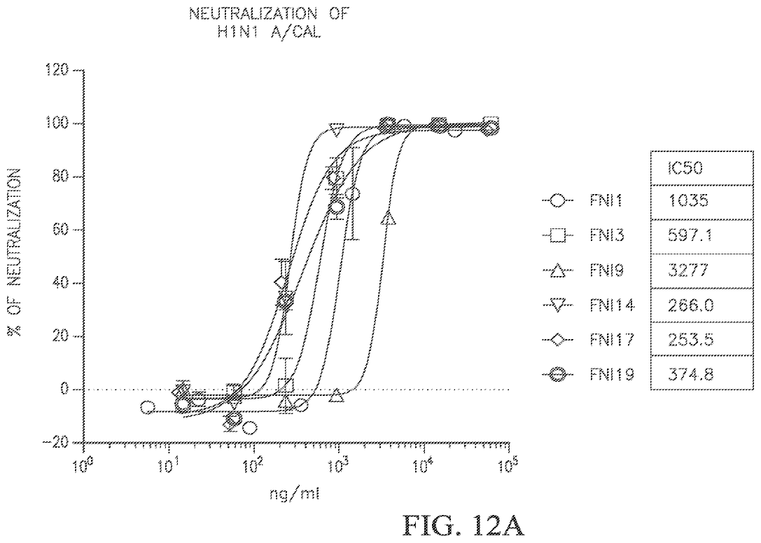

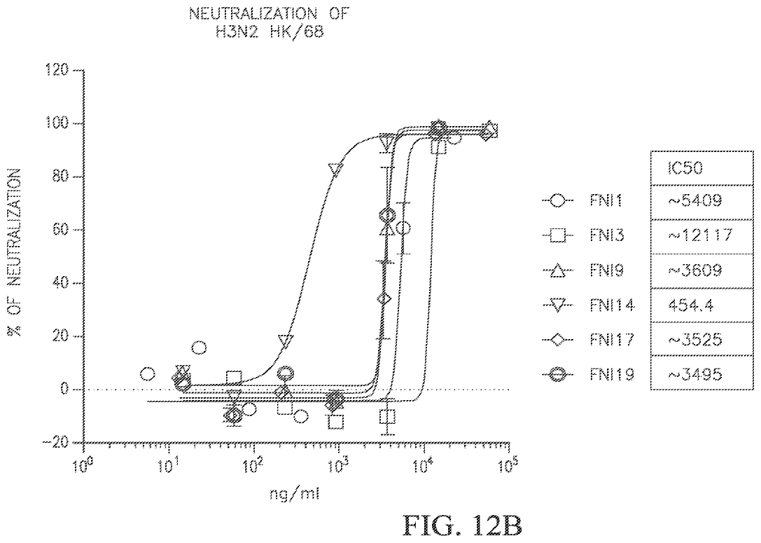

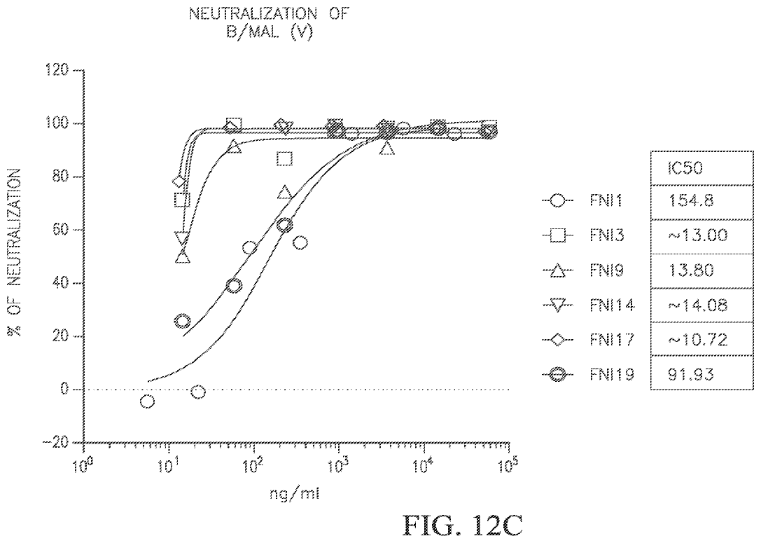

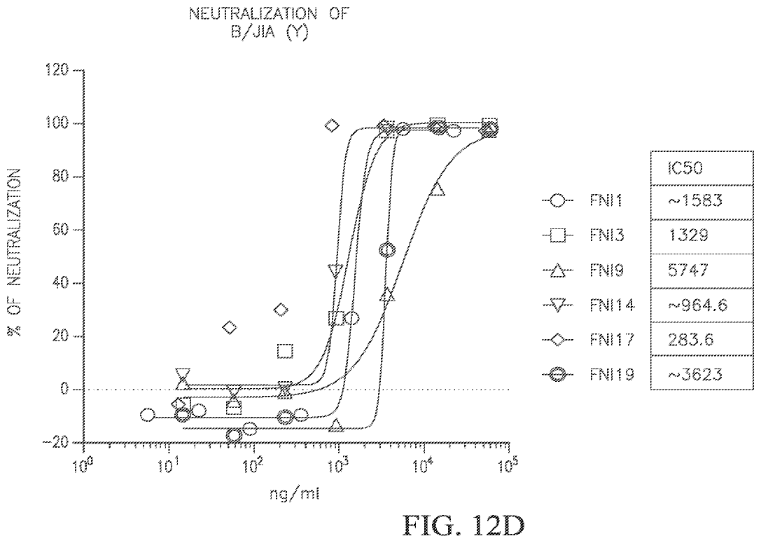

Inhibition of sialidase activity in NAs was measured using a MUNANA assay against group I IAVs, group II IAVs, and IBVs, with results summarized in FIG. 8. Sialidase inhibition of antibody (reported as IC50 in μg/ml) against multiple group I IAVs, group II IAVs, and IBVs strains is summarized in FIG. 9. FIGS. 10A and 10B show in vitro inhibition of sialidase activity (reported as IC50 in μg/ml) by FNI3 or FNI9 against group I (H1N1) IAV, group II (H3N2) IAV, and IBV NAs. FIG. 10A depicts group I IAVs, group II IAVs, and IBVs within the same plot, and FIG. 10B depicts the groups in separate plots. FNI3, FNI9, FNI14, FNI17, and FNI19 were also evaluated for their ability to inhibit sialidase activity (FIG. 11B) of NAs from a panel IAV and IBV strains (FIG. 11A), some of which harbor a glycosylation site at position 245, as indicated by an asterisk. FIGS. 12A-12D show neutralization curves for FNI1, FNI3, FNI9, FNI14, FNI17, and FNI19 against H1N1 A/California/07/2009 (FIG. 12A), H3N2 A/Hong Kong/8/68 (FIG. 12B), B/Malaysia/2506/2004 (FIG. 12C), and B/Jiangsu/10/2003 (FIG. 12D) NAs (reported as IC50 (μg/ml).

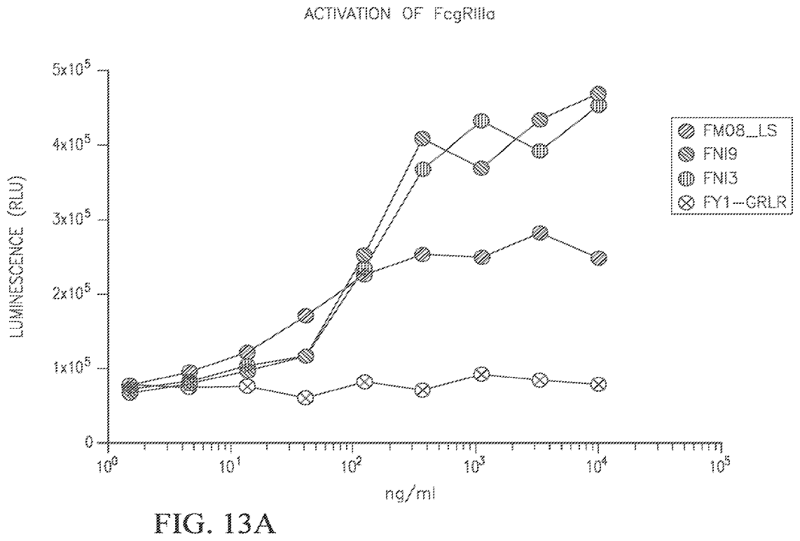

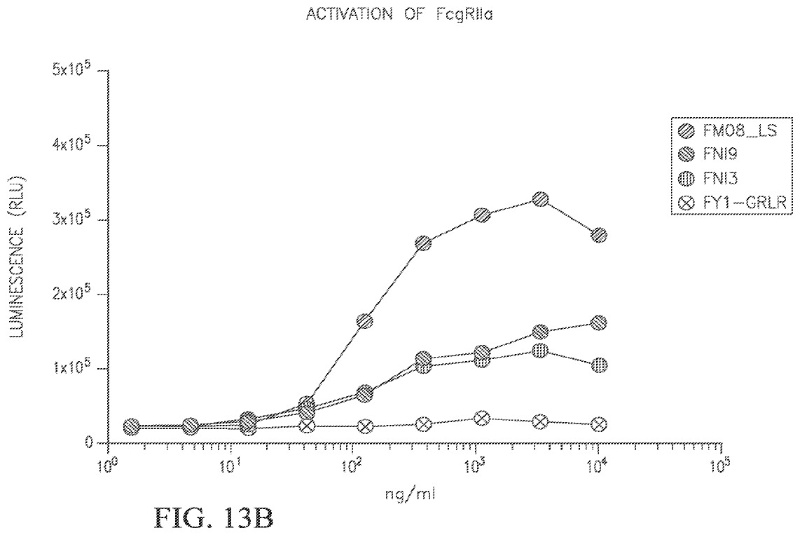

FNI3 and FNI9 were evaluated for activation of FcγRIIIa (FIG. 13A) and FcγRIIa (FIG. 13B) using a NFAT-driven luciferase reporter assay. Activation of Jurkat-FcγRIIIa (F158 allele) and Jurkat-FcγRIIa (H131 allele) cell lines was assessed following a 23 hour incubation with A549 cells infected with H1N1 influenza strain A/Puerto Rico/8/1934 at a multiplicity of infection (MOI) of 6. Comparator antibodies FYT-GRLR and IgG1 antibody FM08_LS, the latter having a VH of SEQ ID NO:25 and a VL of SEQ ID NO:26 and comprising M428L and N434S (EU numbering) Fc mutations, were also tested.

### Example 2

### Structural and Functional Studies of Anti-NA Antibodies

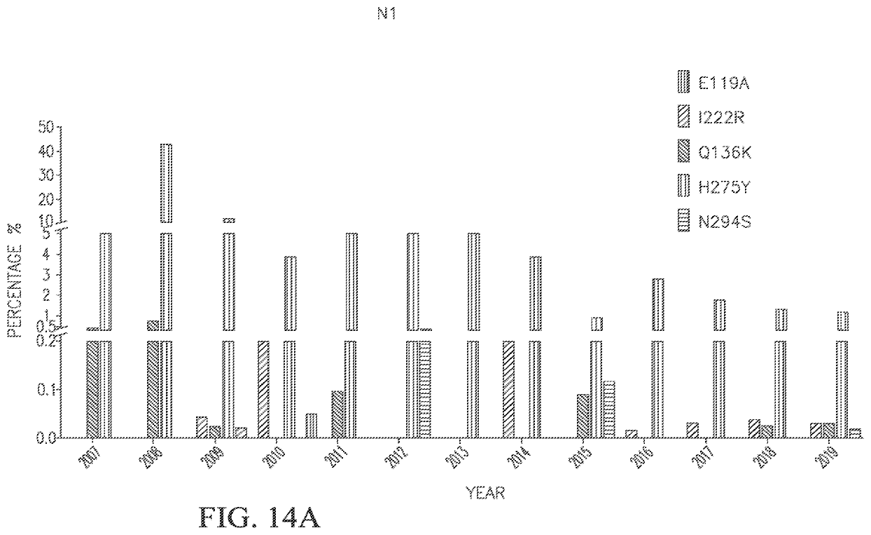

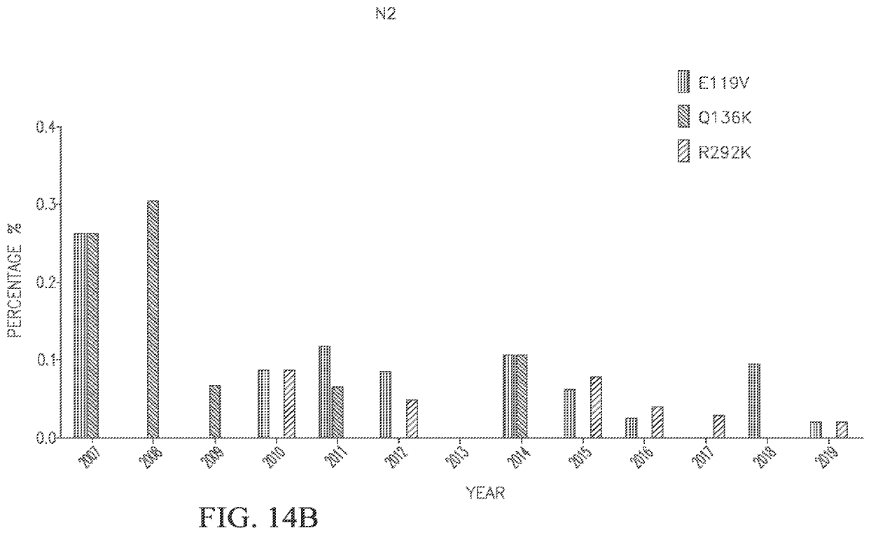

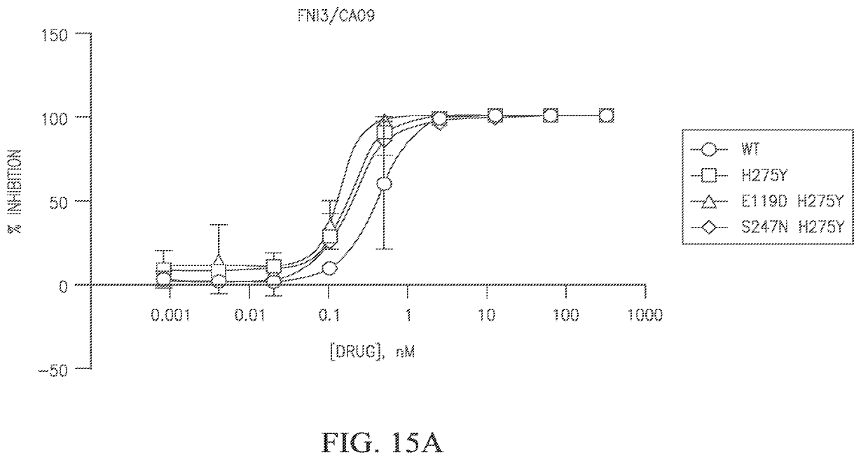

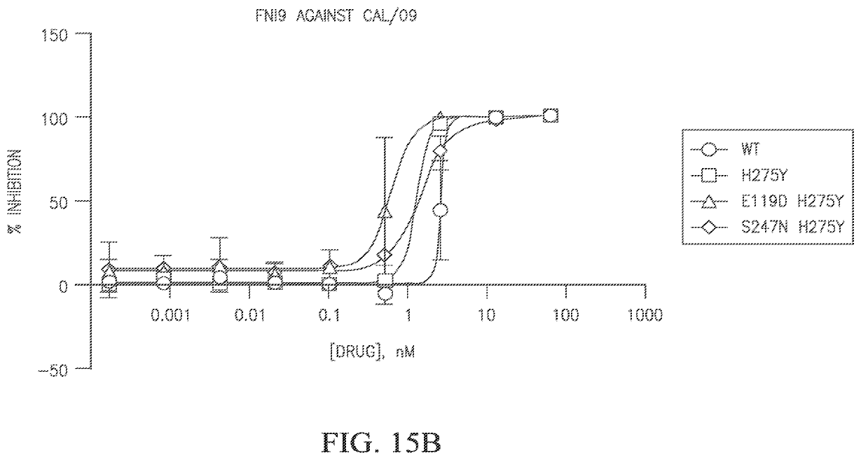

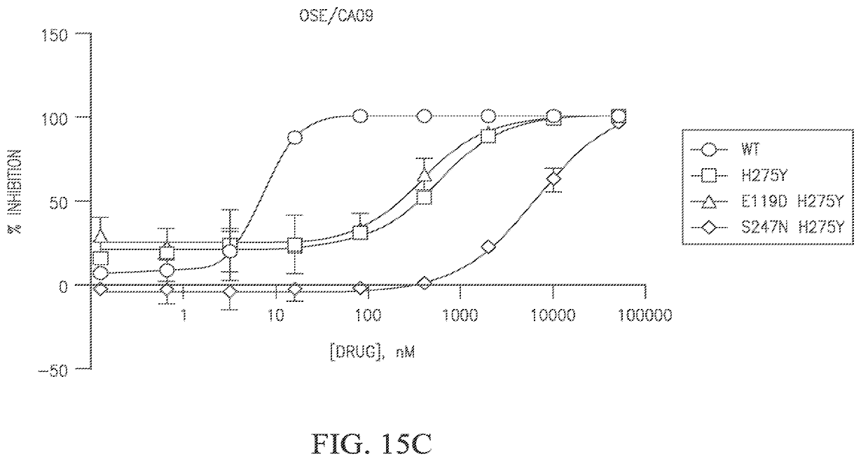

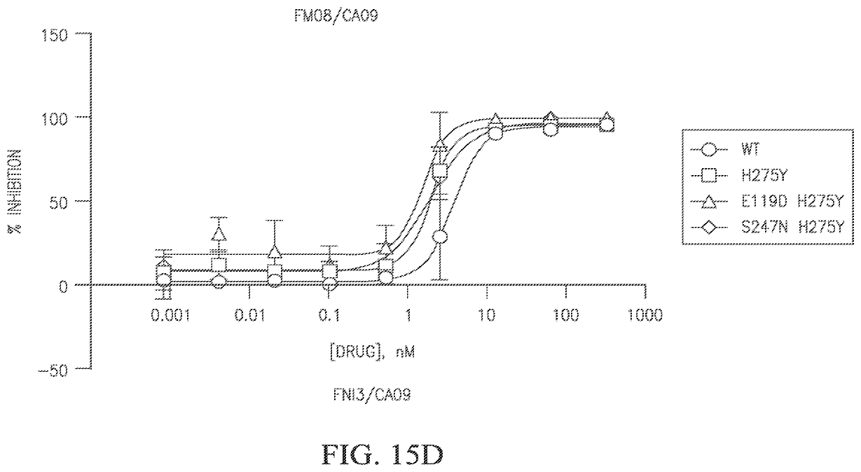

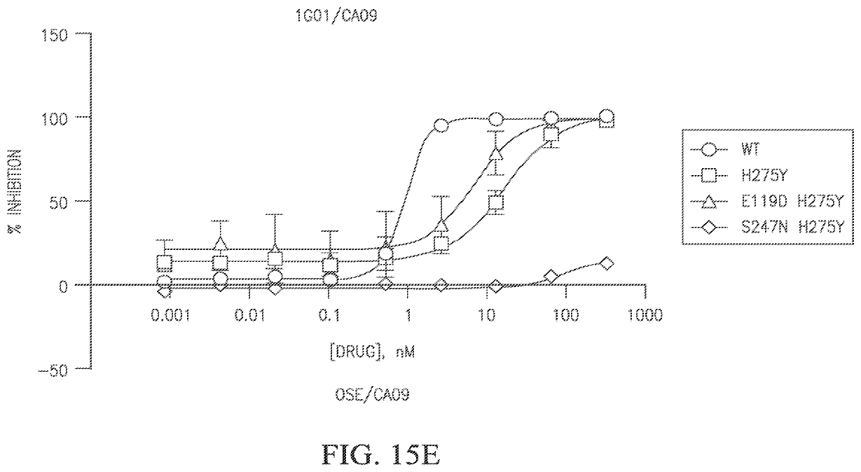

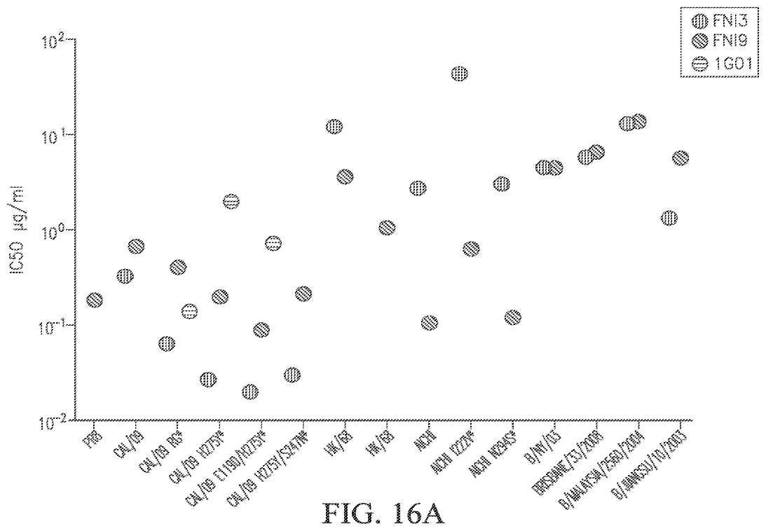

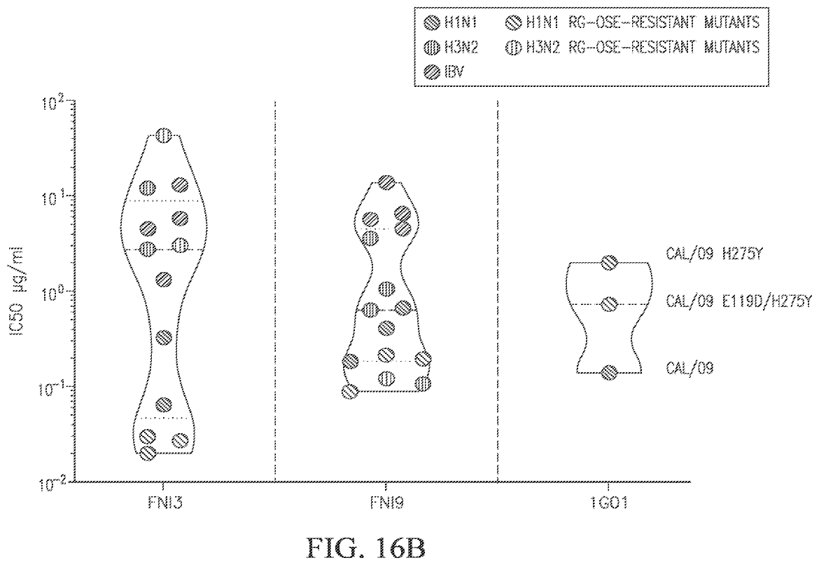

Neuraminidase (NA) mutations responsible for influenza resistance to oseltamivir can vary according to the NA subtype (see, e.g., Hussain et al., Infection and Drug Resistance 10:121-134 (2017)). FIGS. 14A and 14B show frequency by year of NA antiviral-resistant mutations in (FIG. 14A) N1 (H1N1, swine H1N1, and avian H5N1) and (FIG. 14B) N2 (H3N2, H2N2). A reverse genetics approach was used to engineer H1N1 A/California/07/2009 to harbor oseltamivir (OSE)-resistant mutations (H275Y, E119D and H275Y, S247N and H275Y). Neutralization of reverse-engineered H1N1 A/California/07/2009 virus by FNI3 (FIG. 15A), FNI9 (FIG. 15B), and oseltamivir (FIG. 15C) was measured, along with neutralization by comparator antibodies FM08 (FIG. 15D) and 1G01 (FIG. 15E) antibodies and reported as % inhibition in nM. These data suggest a structural basis for the lack of susceptibility of FNI3 and FNI9 to OSE-resistant NA mutations. Next, additional viruses, including Group I (H1N1) IAV, group II (H3N2) IAV, and IBV viruses were engineered with reverse genetics to bear OSE-resistant mutations (H275Y, E119D/H275Y, H275Y/S247N, 1222V, and N294S). Neutralization activity of FNI3, FNI9, and comparator antibody 1G01 was measured and reported as IC50 in μg/ml. FIG. 16A depicts neutralization of individual viral strains and FIG. 16B depicts neutralization of viral strains grouped by neutralizing anti-NA antibody.

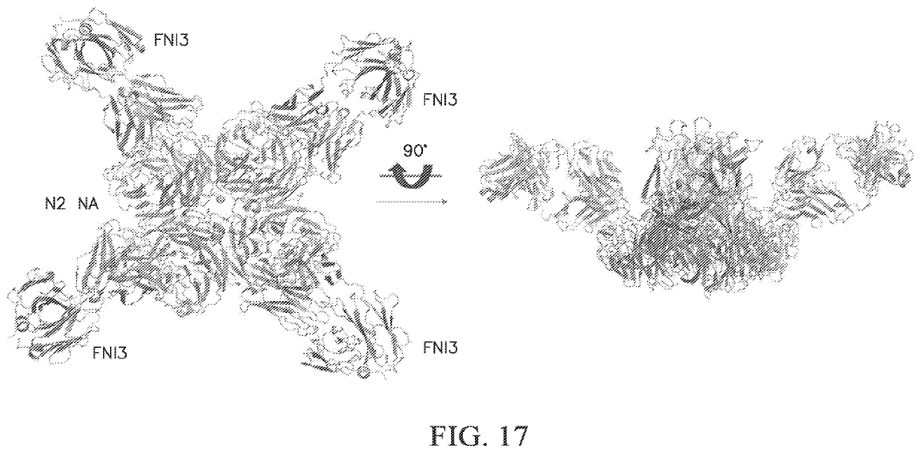

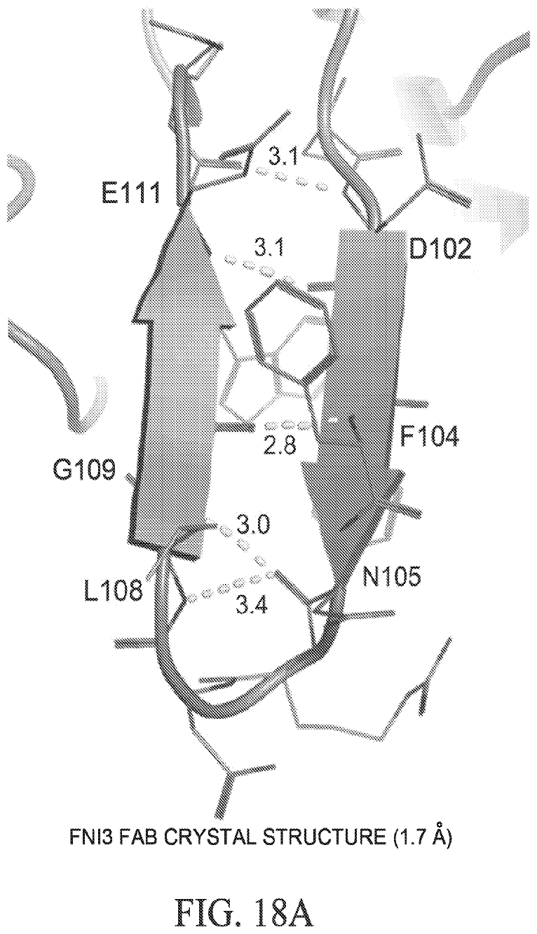

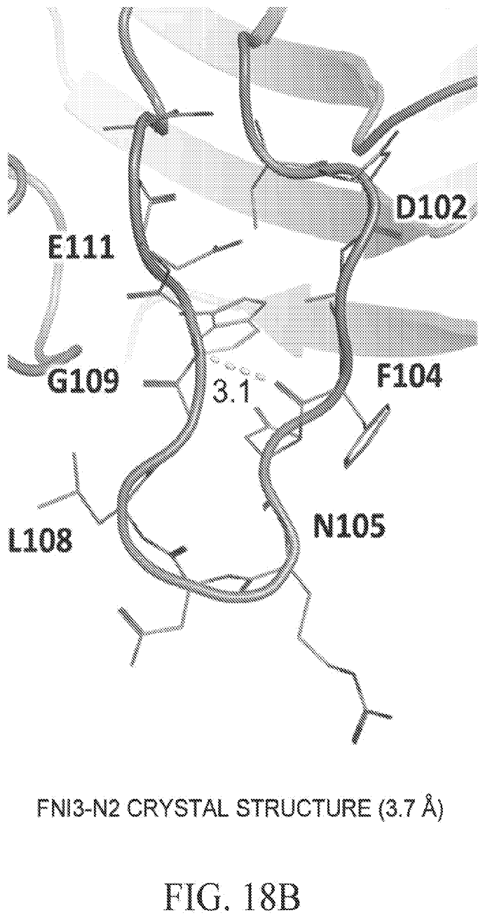

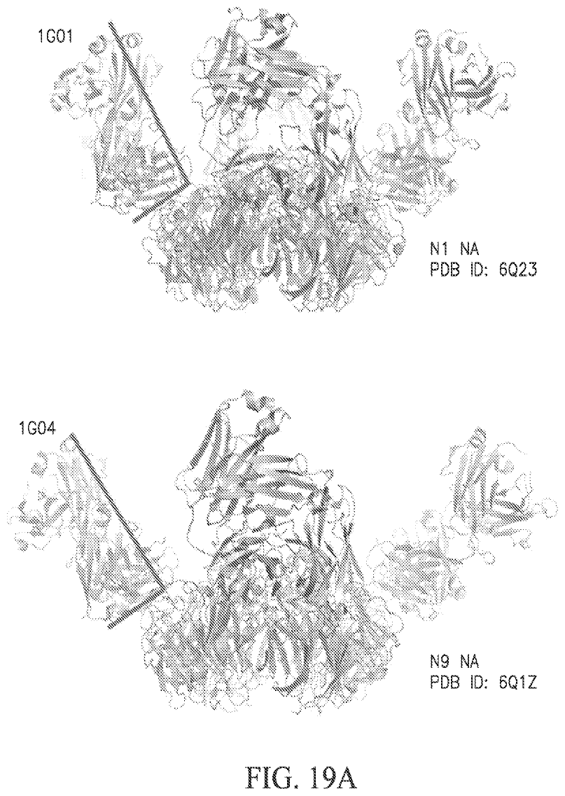

The crystal structure of FNI3 (alone or in complex with NA) was determined to investigate binding function. A relatively flat docking angle of the FNI3 antigen-binding fragment (Fab) domain in complex with NA is shown in FIG. 17. Crystal structure analysis of the complementarity-determining region 3 (CDR3) of the FNI3 heavy chain was performed for unbound (FIG. 18A) or N2 NA-bound states (FIG. 18B). From these studies, unbound FNI3 crystal structure (FIG. 18A) shows a beta sheet conformation and intact main chain hydrogen bonds between carboxylic acid groups (CO) and amino groups (NH) of residues E111 (CO)-D102 (NH), E111 (NH)-D102 (CO), G109 (CO)-F104 (NH), G109 (NH)-N105 (CO), and L108 (NH)-N105 (CO); bound FNI3-N2 crystal structure (FIG. 18B) shows disruption of the beta sheet conformation and one intact main chain hydrogen bond between G109 (CO)-F104 (NH). Without being bound by theory, absence of beta sheet structure in the FNI3-N2 crystal structure might be explained by two potential scenarios: (1) disruption of beta sheet may occur due to induced fit by binding to N2 NA; (2) beta sheet formation may occur due to induced fit by crystal contacts for the Fab domain alone.

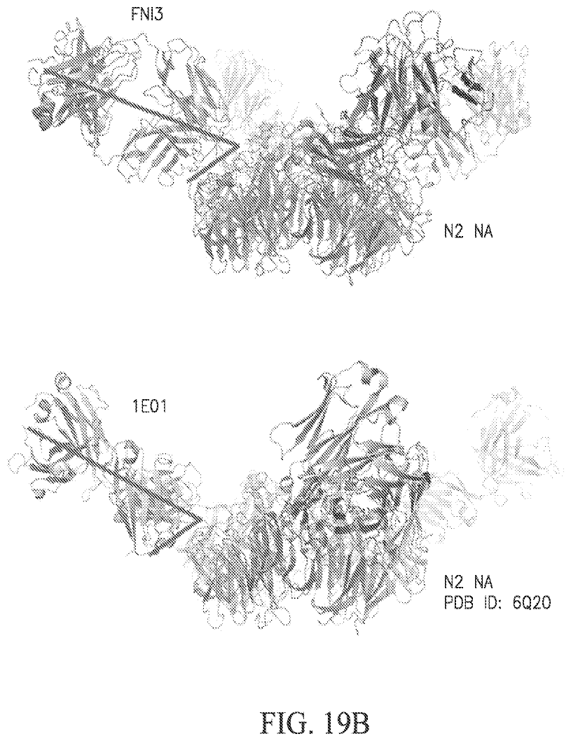

Crystal structure and angle of docking of the Fab domain of the FNI3 antibody in complex with NA subtypes was compared to analogous properties of other anti-NA antibodies to further characterize docking properties of FNI3. FIG. 19A shows comparator antibodies: 1G01 in complex with N1 NA (upper panel); and 1G04 (Stadlbauer et al., supra) in complex with N9 NA (lower panel). FIG. 19B shows FNI3 in complex with N2 NA (upper panel) wherein the docking angle is the same as shown in FIG. 17, but the Fab domain is in a different orientation. FIG. 19B also shows a comparator antibody, 1E01 (Stadlbauer et al., supra), in complex with N2 NA (lower panel). Lines indicate angle of docking and Protein Data Bank (PDB) identification codes are shown for comparator antibodies. From these studies, FNI3 has a similar docking angle to 1E01, but a different Fab orientation.

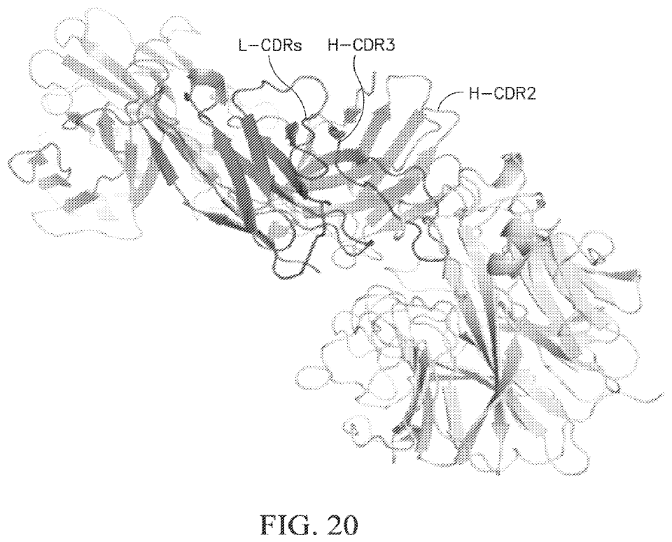

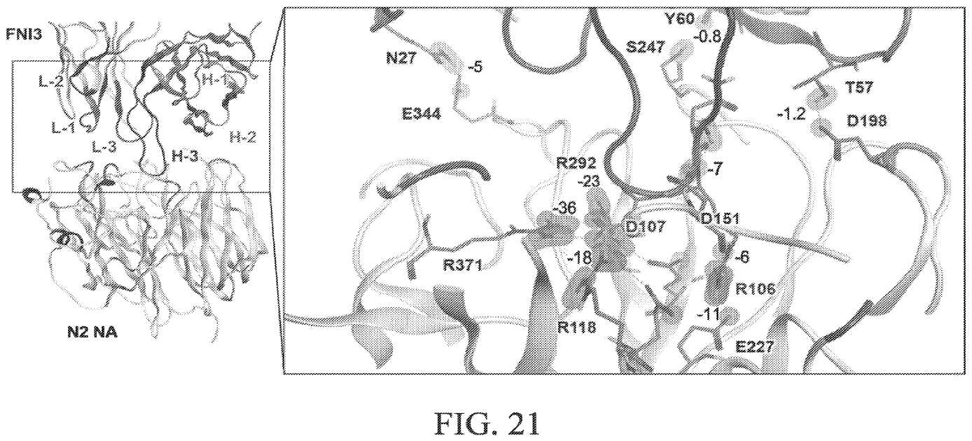

FNI3 complementarity-determining region (CDR) interactions are shown schematically in FIG. 20, from “quick prepped” protein using MOE (Molecular Operating Environment by the Chemical Computing Group; www.chemcomp.com). From this analysis, CDRH3 bends at an almost 90° angle to occupy the NA binding pocket and CDRH2 lays flat on the NA surface. From this analysis, CDRH2 does not appear to energetically contribute to binding and CDRLs do not appear to contribute to the binding interaction. From this study, within the CDRH3, D107 and R106 appear to contribute to N2 NA binding (FIG. 21). Negative numbers are interaction energy in kcal/mol.

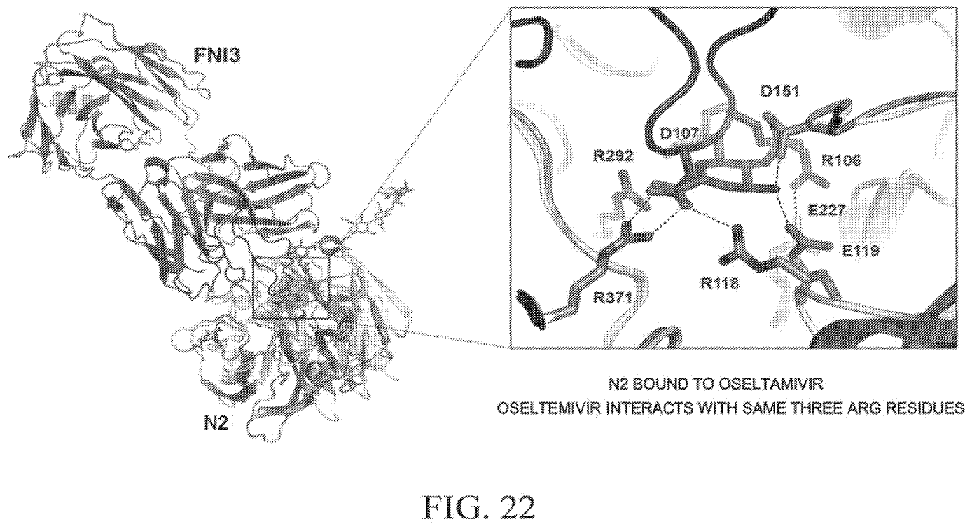

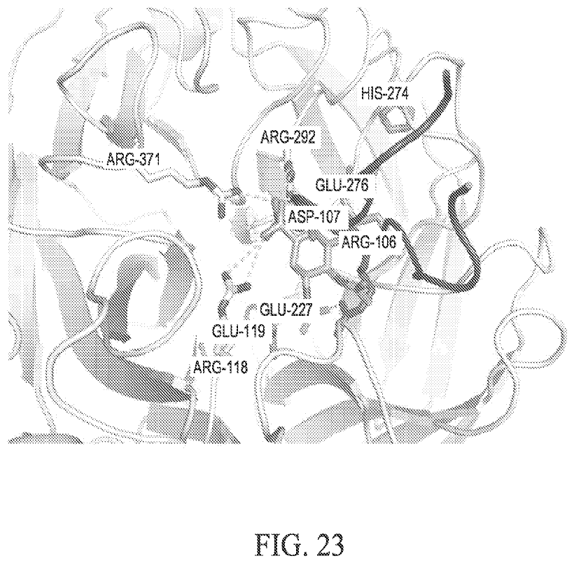

The crystal structure of FNI3 was overlaid on the structure of oseltamivir-bound N2 NA (FIG. 22, FIG. 23), showing that oseltamivir interacts with R118, R292, and R371.

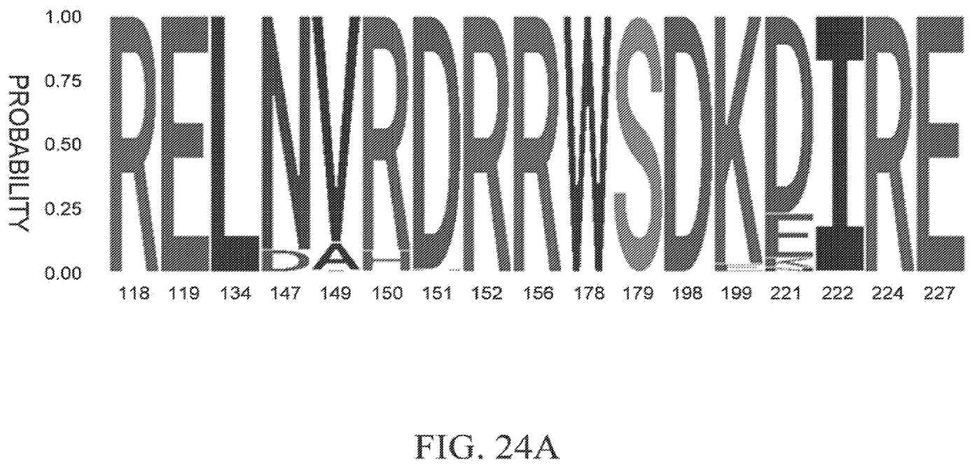

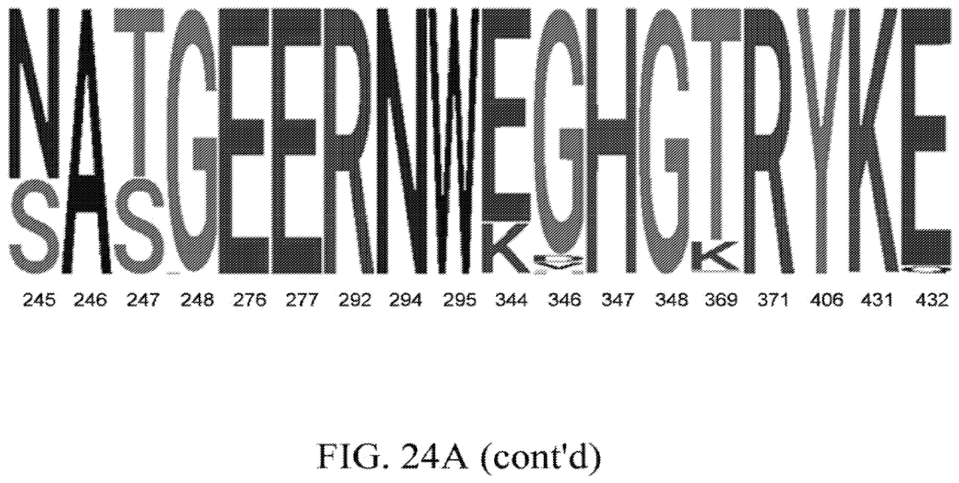

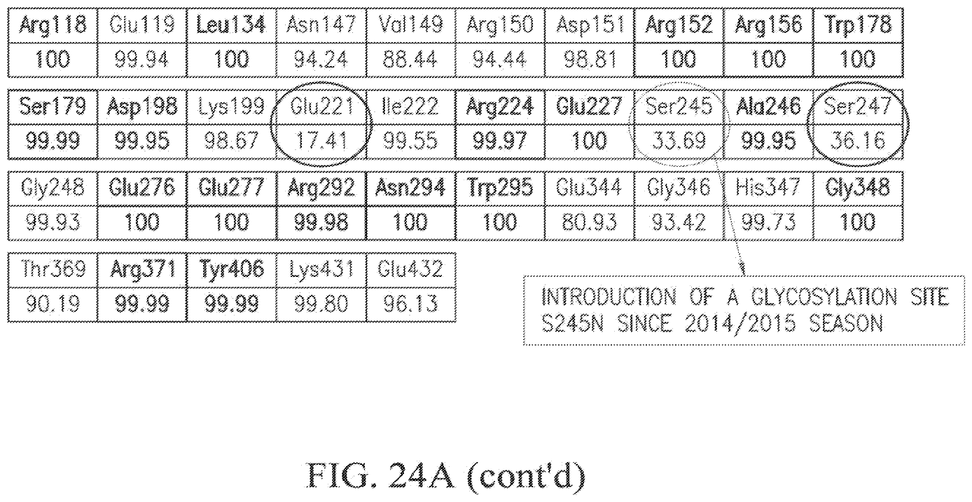

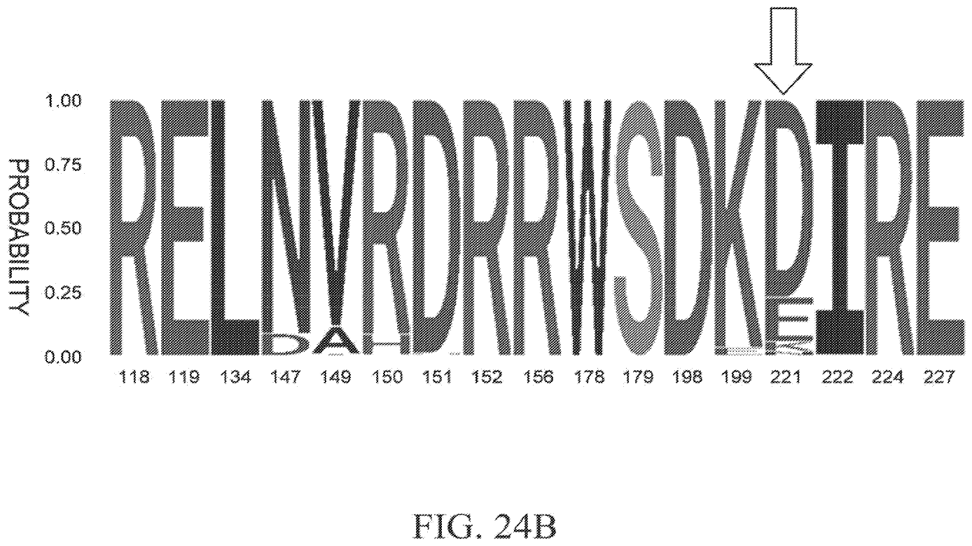

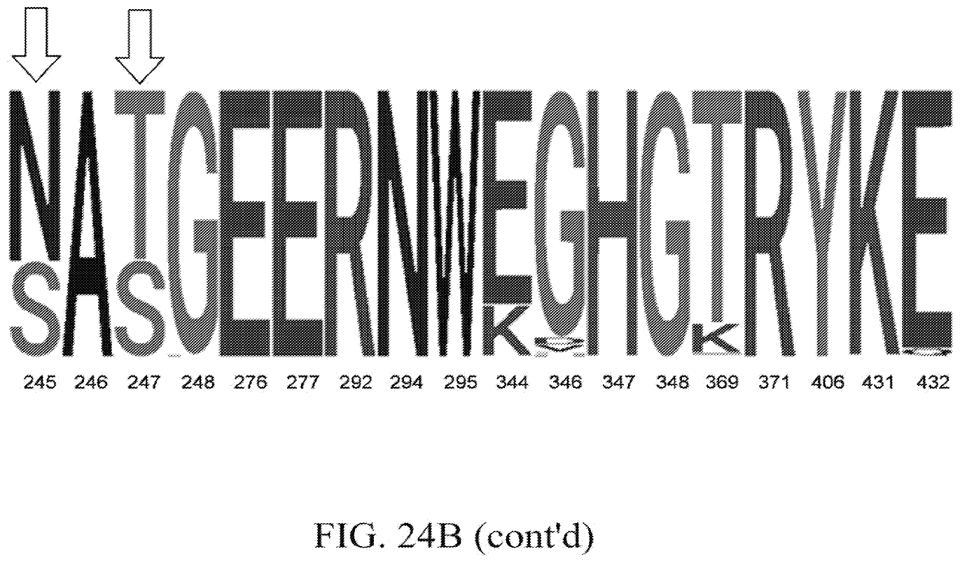

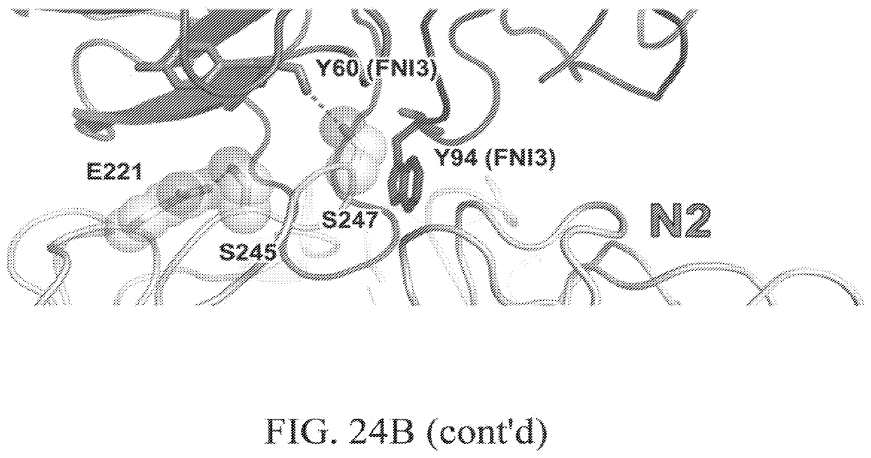

Conservation of the FNI3 epitope was investigated using N2 NA sequences from H3N2 viruses (n=60,597) isolated between the years 2000 and 2020. The epitope region consensus amino acid sequence is shown in FIG. 24A, with a table showing the frequency of an amino acid at a particular position in the group of analyzed N2 NA sequences. Circled values indicate amino acids appearing at the lowest three frequencies, Glu221 (E221, 17.41%), Ser245 (S245, 33.69%), and Ser247 (S247, 36.16%). FIG. 24B (lower portion) shows interaction of Y60 and Y94 from FNI3 with residues E221, S245, and S247 of N2 NA. Using simple modeling, a S245N mutation increased binding, a S247T mutation decreased binding, and a E221D mutation was neutral in effect (data not shown).

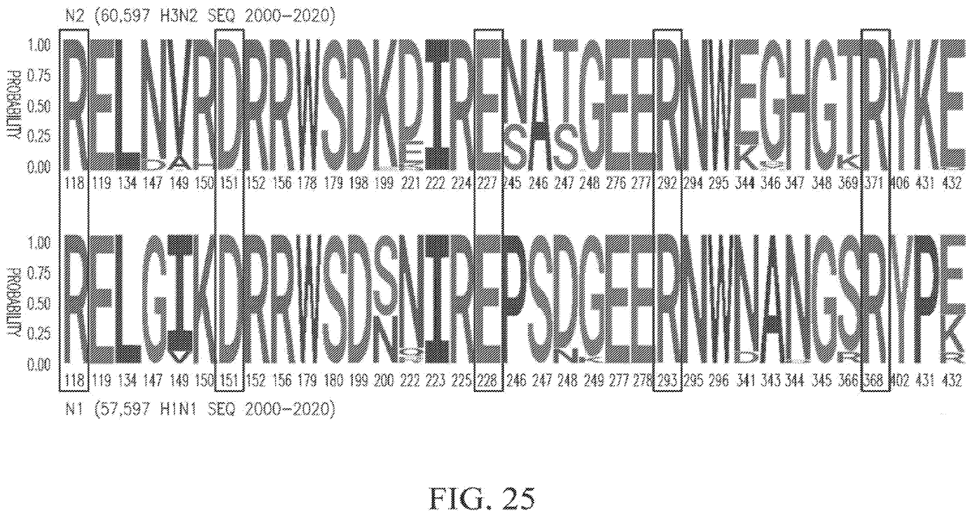

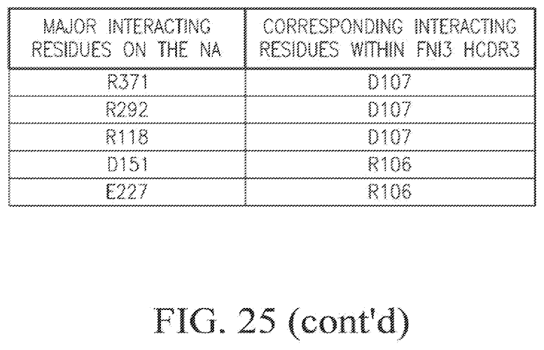

Conservation of the FNI3 epitope was investigated using N1 NA sequences from H1N1 viruses (n=57,597) isolated between the years 2000 and 2020. FIG. 25 shows a comparison of N2 NA FNI3 epitope conservation analysis (shown in FIGS. 24A and 24B) with analysis of FNI3 epitope conservation in N1 NA sequences from H1N1. Pairs of consensus residues were identified, R118 (N2) and R118 (N1), D151 (N2) and D151 (N1), E227 (N2) and E228 (N1), R292 (N2) and R293 (N1), and R371 (N2) and R368 (N1). Important FNI3-interacting residues within N2 NA and counterpart FNI3 CDRH3 residues are shown in the table in the lower panel. Residues R371, R292, and R118 interact with D107 of FNI3 CDRH3 and residues D151 and E227 interact with R106 of FNI3 CDRH3.

### Example 3

### Prophylactic Activity of Anti-Na Monoclonal Antibodies

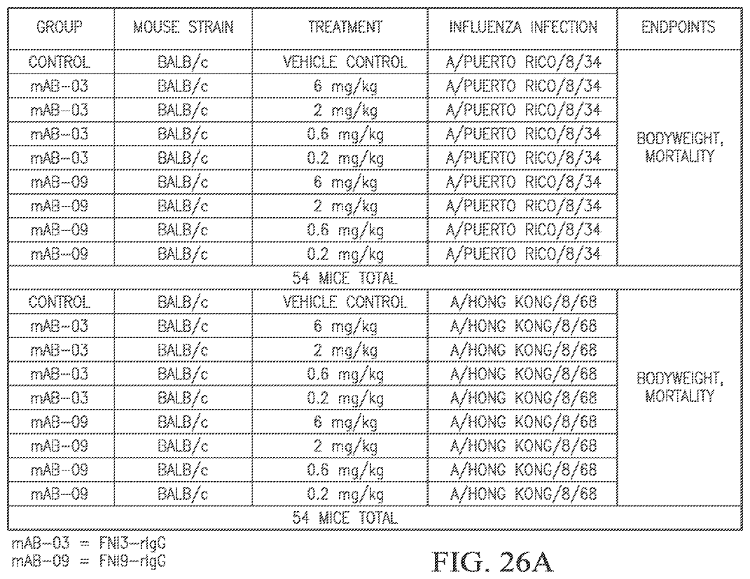

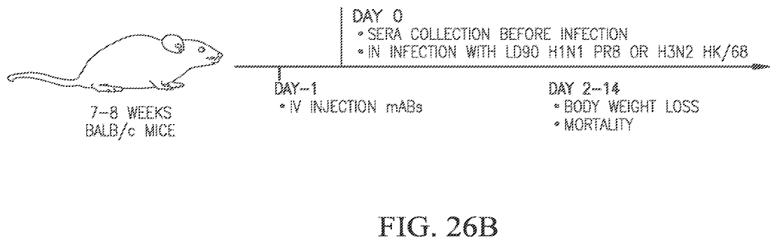

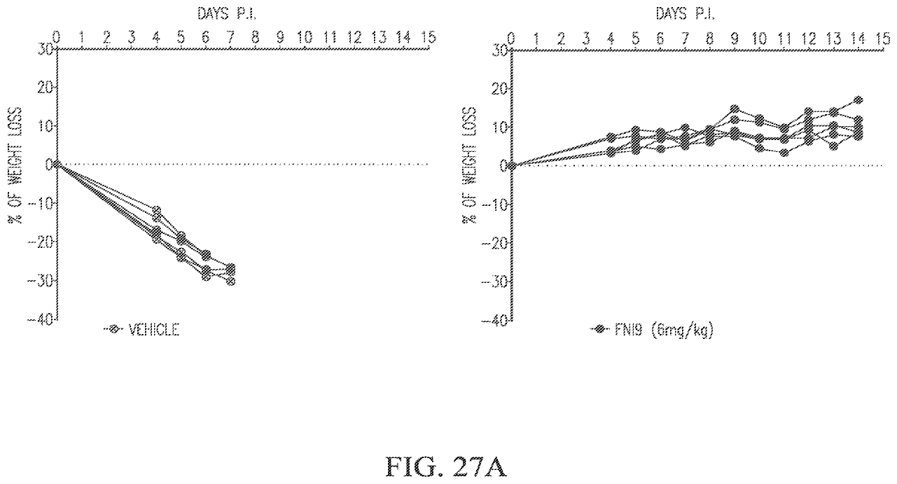

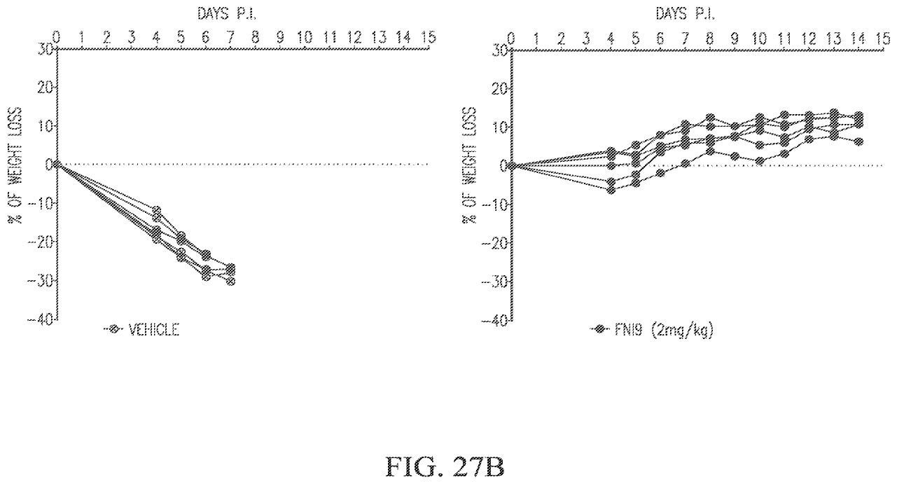

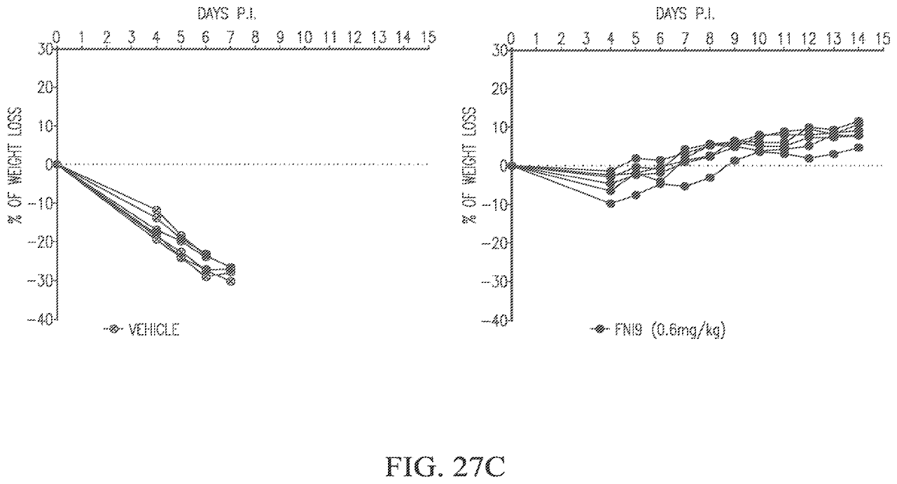

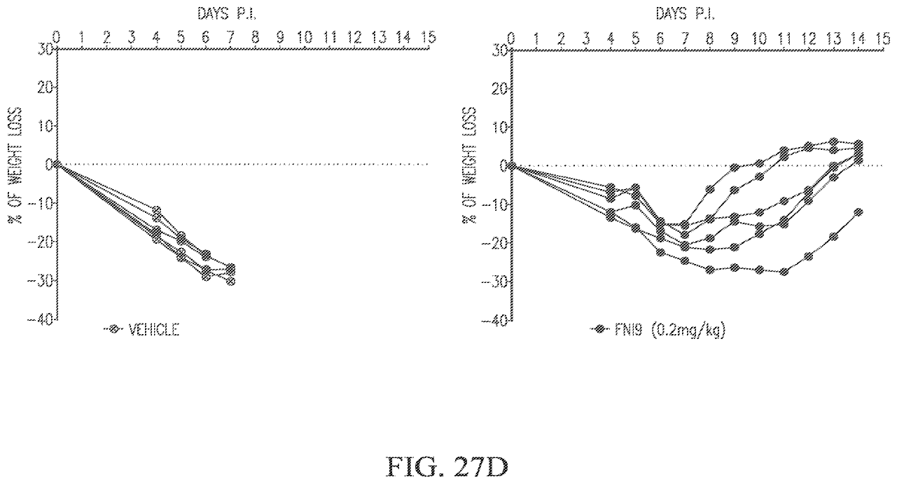

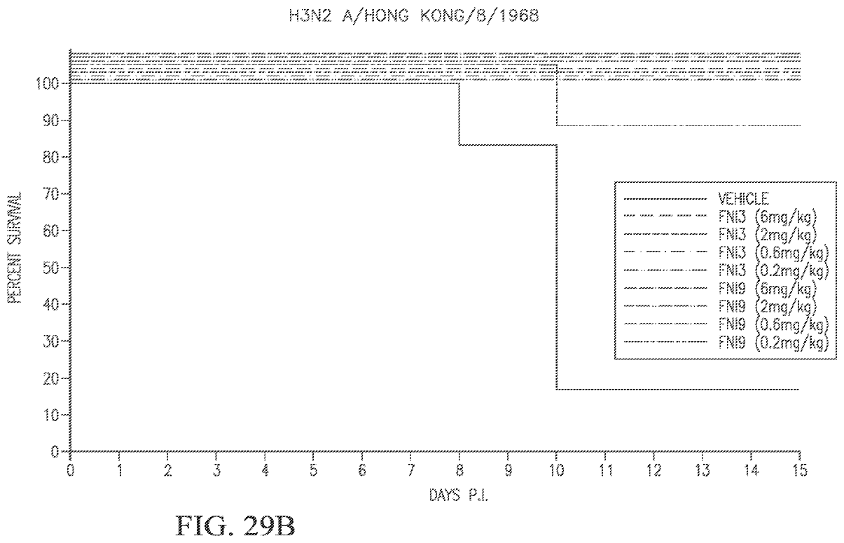

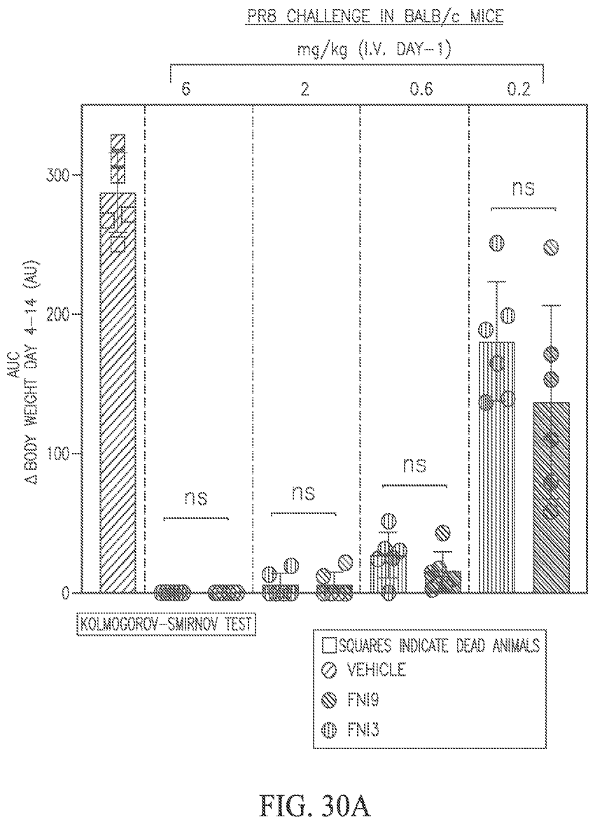

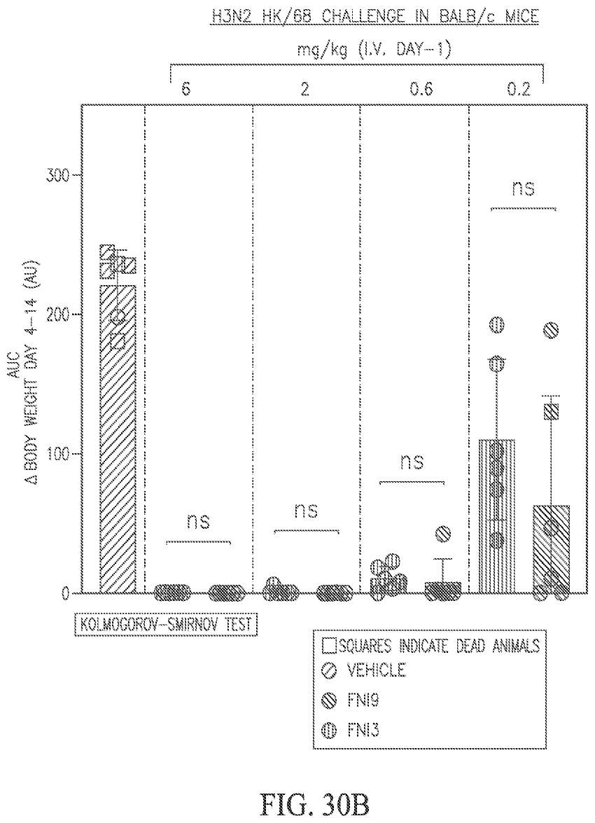

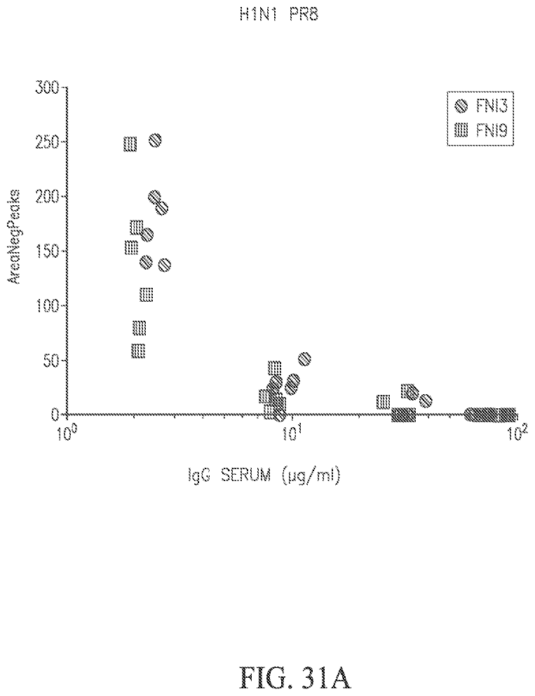

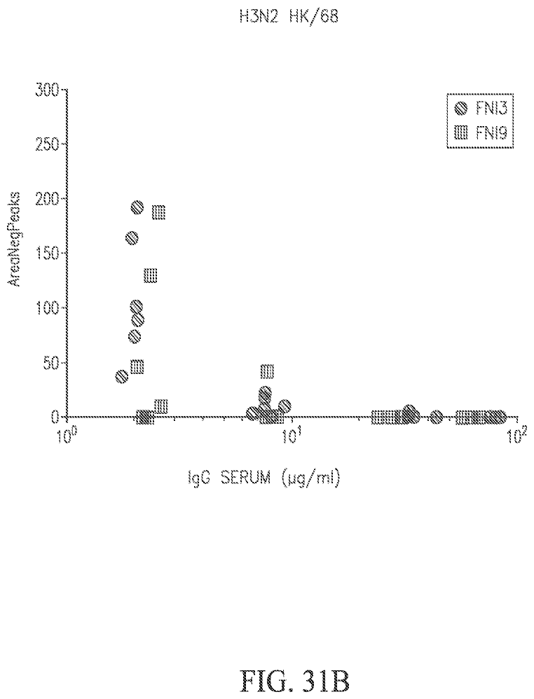

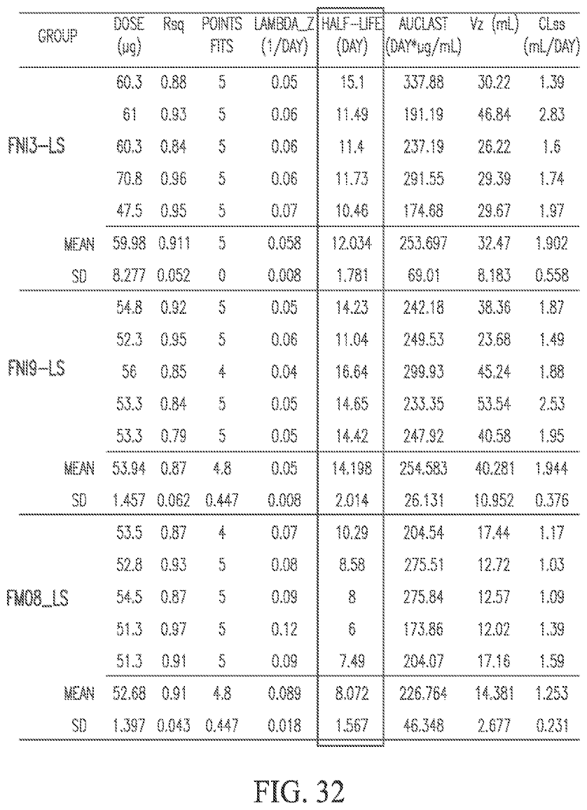

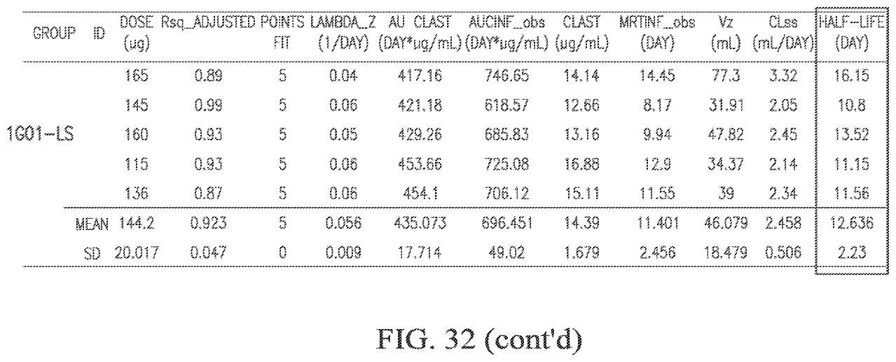

Prophylactic activity of FNI3 and FNI9 was evaluated in a murine BALB/c model of IAV infection. Briefly, BALB/c mice, 7-8 weeks of age, were administered (i.v.) FNI3 (“mAb-03” in FIG. 26A), FNI9 (“mAb-09” in FIG. 26A), or vehicle control one day prior to intranasal infection at LD90 (90% of a lethal dose) with H1N1 subtype A/Puerto Rico/8/34 or H3N2 subtype A/Hong Kong/1/68 (FIGS. 26A and 26B). Antibody was administered (i.v.) at 0.2. 0.6, 2, or 6 mg/kg. Baseline serum was collected at the start of infection, and both body weight and mortality were evaluated on each of Days 2-14 post-infection (FIG. 26B). Body weight measurements for certain of the test conditions over fifteen days are shown in FIGS. 27A-27D (A/Puerto Rico/8/34 FNI9 test group) and 28A-28D (A/Hong Kong/1/68 FNI9 test group). Administration of FNI3 (data not shown) or FNI9 reduced body weight loss (as compared to vehicle) at all antibody doses (0.2. 0.6, 2, and 6 mg/kg). Overall mortality was also measured (FIG. 29A, A/Puerto Rico/8/34-infected mice; FIG. 29B, A/Hong Kong/1/68-infected mice). FIGS. 30A and 30B show body weight loss reported as area-under-the-curve in mice infected with A/Puerto Rico/8/34 (FIG. 30A) or A/Hong Kong/8/68 (FIG. 30B). Negative area-under-the-curve peaks compared with IgG in serum from area-under-the-curve analyses of body weight loss in BALB/c mice infected with A/Puerto Rico/8/34 (FIG. 31A) or A/Hong Kong/8/68 (FIG. 31B) are also shown. Pharmacokinetics of FNI3 (“FNI3-LS”), FNI9 (“FNI9-LS”) and comparator antibodies FM08_LS and 1G01 (“1G01-LS”) in tg32 mice is shown in FIG. 32.

### Example 4

### Pharmacokinetic Study

Pharmacokinetic analysis of Fc variants (M428L/N434S mutations) of FNI3 (“FNI3-LS”), FNI9 (“FNI9-LS”), and comparator antibodies FM08_LS and 1G01-LS was assessed in tg32 mice, and half-life was performed, with results summarized in FIG. 32. Plasma concentration of the antibodies was determined in vitro using an ELISA assay. Goat anti-human IgG antibody (Southern Biotechnology: 2040-01) was diluted to 10 μg/ml in PBS and 25 μl was added to the wells of a 96-well flat bottom ½-area ELISA plate for coating over night at 4° C. After coating, the plates were washed twice with 0.5×PBS supplemented with 0.05% Tween20 (wash solution) using an automated ELISA washer. Then, plates were blocked with 100 μl/well of PBS supplemented with 1% BSA (blocking solution) for 1 h at room temperature (RT) and then washed twice. Samples were then diluted 1:2 stepwise in duplicates for a total of 8 dilutions. Standards for each antibody to be tested were prepared similarly via diluting the antibodies to 0.5 μg/ml. Standards were then diluted 1:3 stepwise in blocking solution in duplicates for a total of 8 dilutions. Twenty-five μl of the prepared samples or standards were added to Goat anti human IgG-coated wells and incubated for 1 h at RT. After four washes, 25 μl of polyclonal anti-IgG-alkaline phosphatase conjugated antibodies (Southern Biotechnology: 2040-04) diluted in blocking solution 1:500 were added per well for detection and incubated at RT for 1 h. After four washes, plates were developed by adding 80 μl/well of substrate solution (1 tablet of p-NitroPhenyl Phosphate (Sigma-Aldrich: N2765-100TAB) in 20 ml bicarbonate buffer). After 30 min incubation at RT, the absorbance was measured at 405 nm using a spectrophotometer.

To determine the concentration of the antibodies in mouse plasma, OD values from ELISA data were plotted vs. concentration in Gen5 software (BioTek). A non-linear curve fit was applied using a variable slope model, four parameters, and the equation: Y=(A−D)/(1+(X/C)\hat{ }B)+D). The OD values of the sample dilutions that fell within the predictable assay range of the standard curve % as determined in setup experiment by quality control samples in the upper, medium, or lower range of the curve % were interpolated to quantify the samples. Plasma concentration of the antibodies were then determined considering the final dilution of the sample. If more than one value of the sample dilutions fell within the linear range of the standard curve, an average of these values was used. Pharmacokinetics (PK) data were analyzed by using WINNONLIN NONCOMPARTMENTAL ANALYSIS PROGRAM (8.1.0.3530 Core Version, Phoenix software, Certara) with the following settings: Model: Plasma Data, i.v. Bolus Administration; Number of non-missing observations: 8; Steady state interval Tau: 1.00; Dose time: 0.00; Dose amount: 5.00 mg/kg; Calculation method: Linear Trapezoidal with Linear Interpolation; Weighting for lambda_z calculations: Uniform weighting; Lambda_z method: Find best fit for lambda_z, Log regression. Graphing and statistical analyses (linear regression or outlier analysis) were performed using Prism 7.0 software (GraphPad, La Jolla, CA, USA).

### Example 5

### Generation of FNI3 and FNI9 Variant Antibodies

Variants of FNI3 and FNI9 were generated by mutating amino acids in the variable regions. See Tables 1 and 2.

### Example 6

### Additional Studies

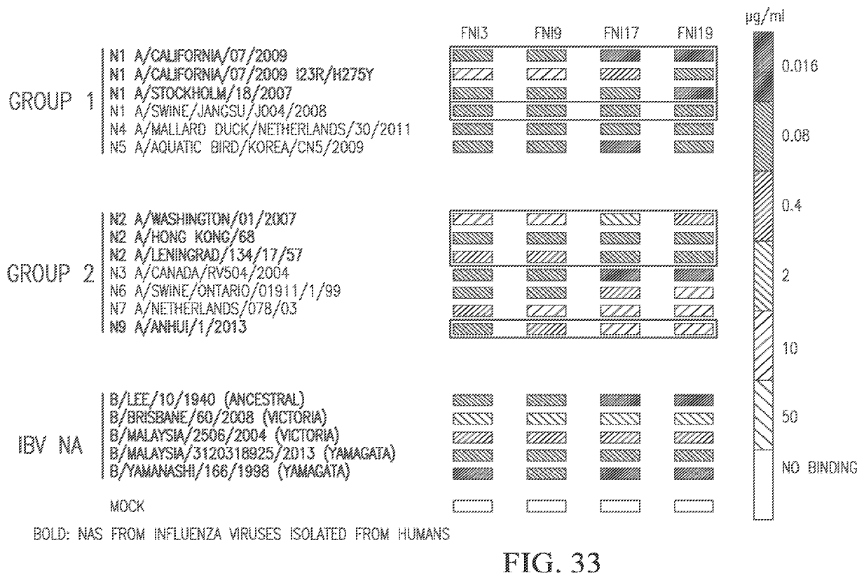

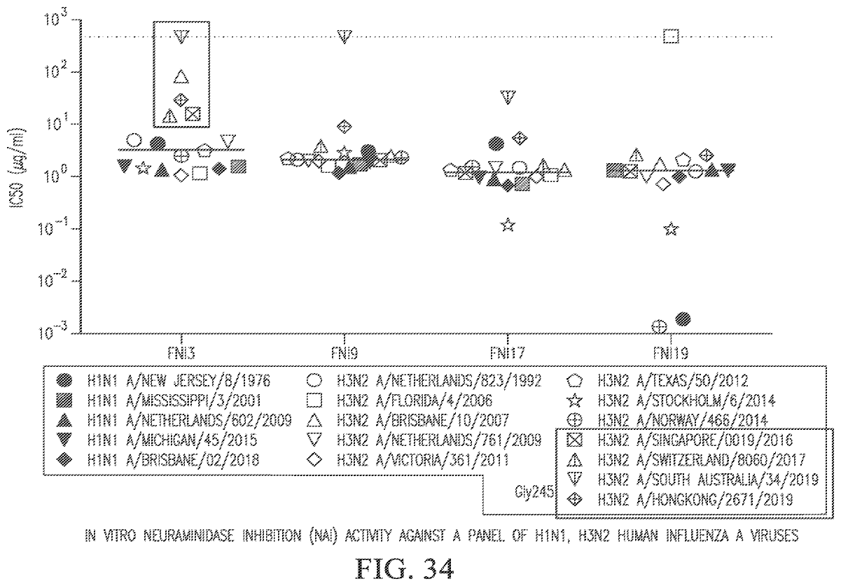

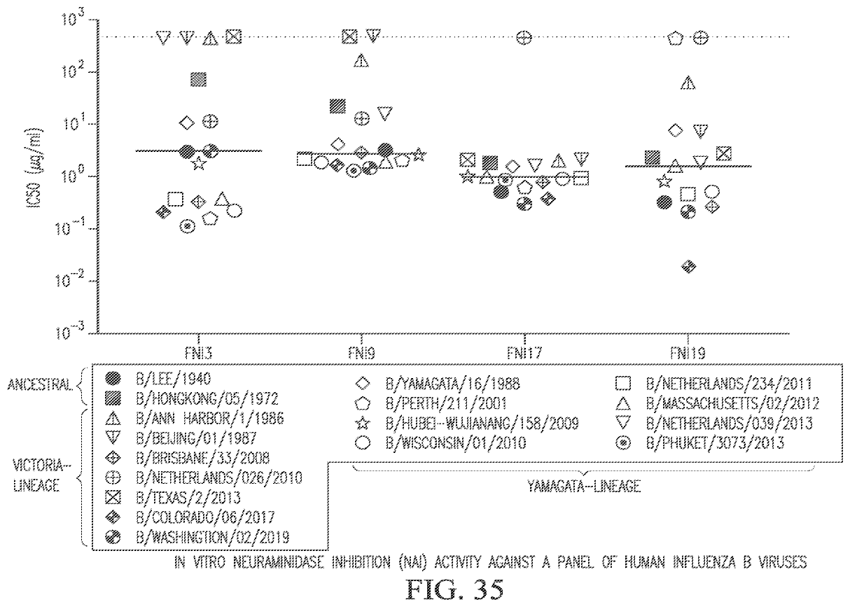

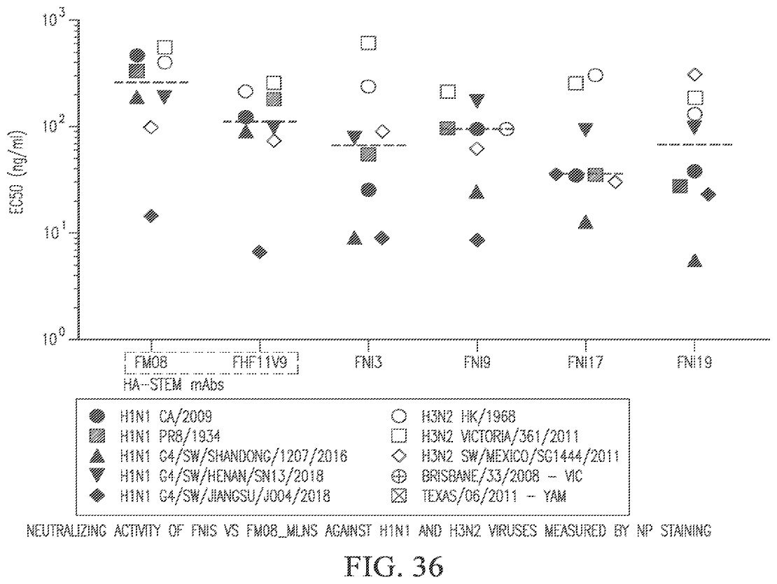

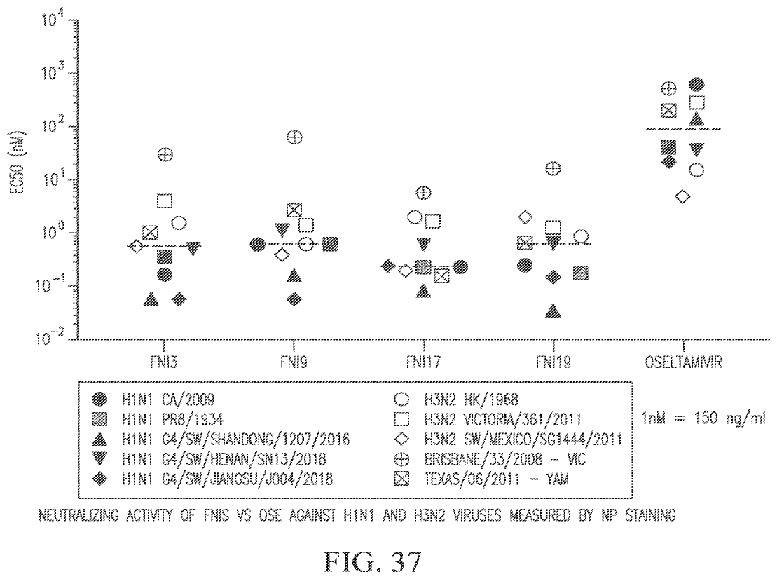

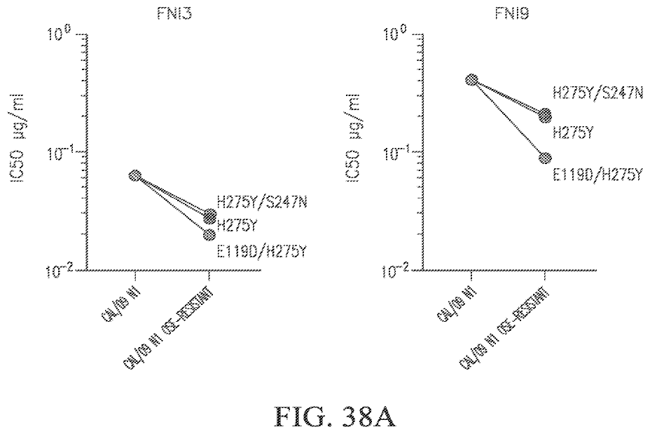

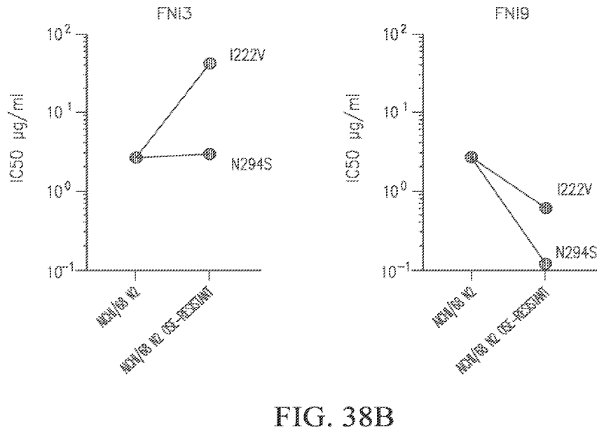

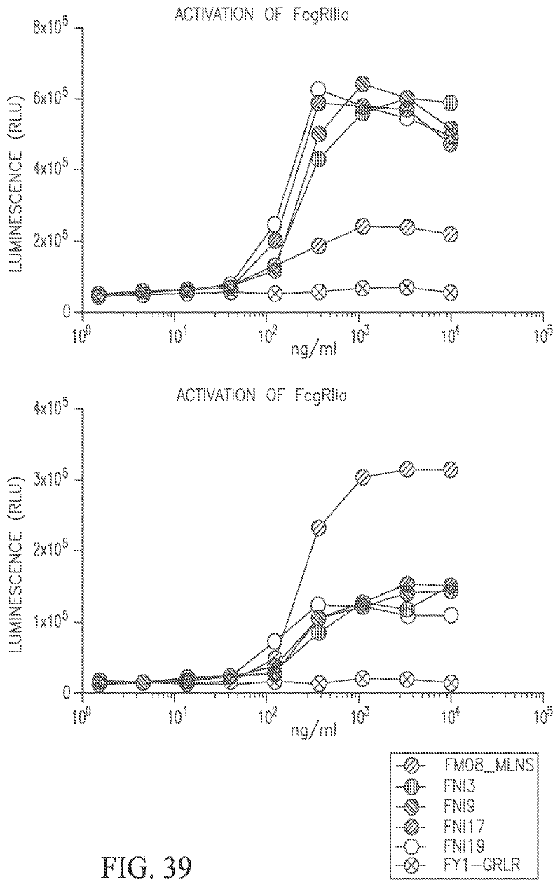

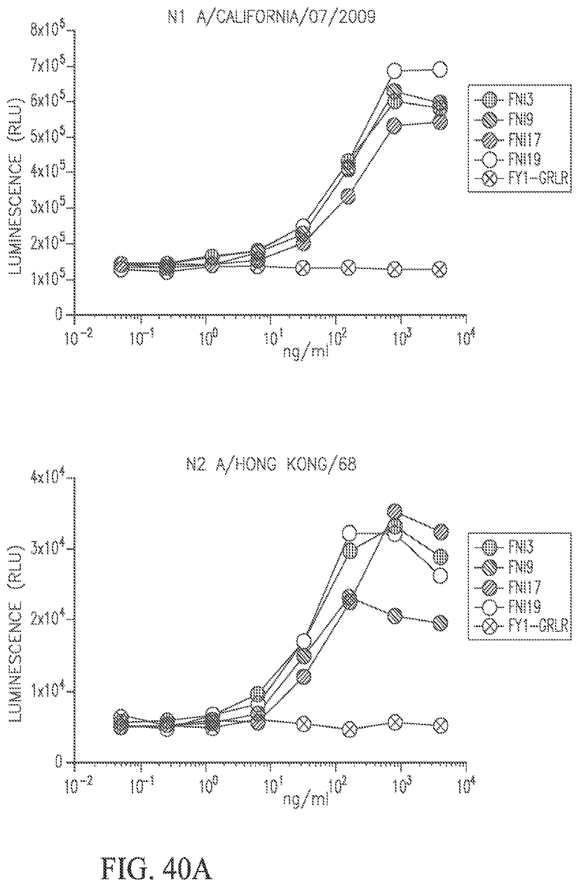

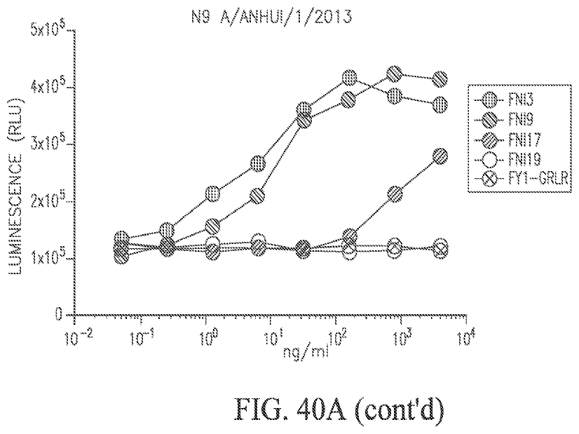

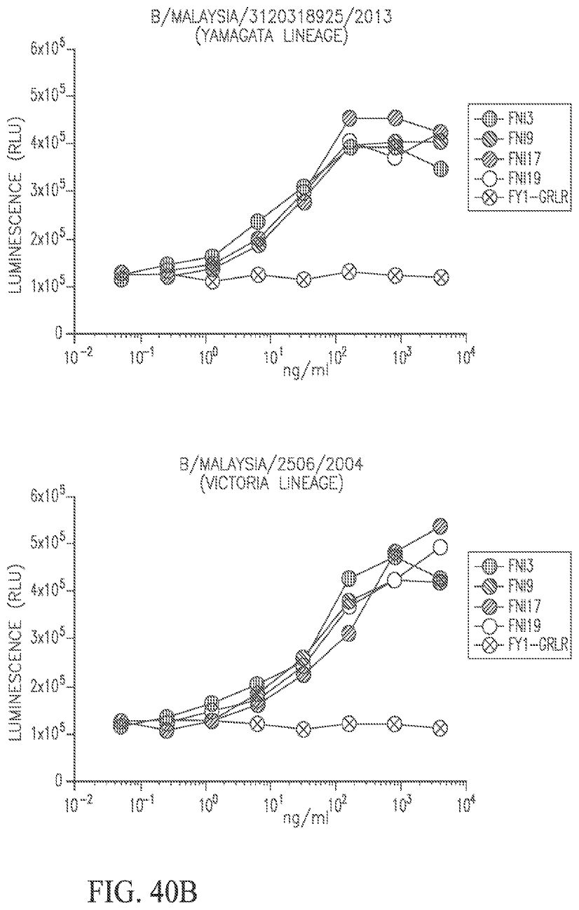

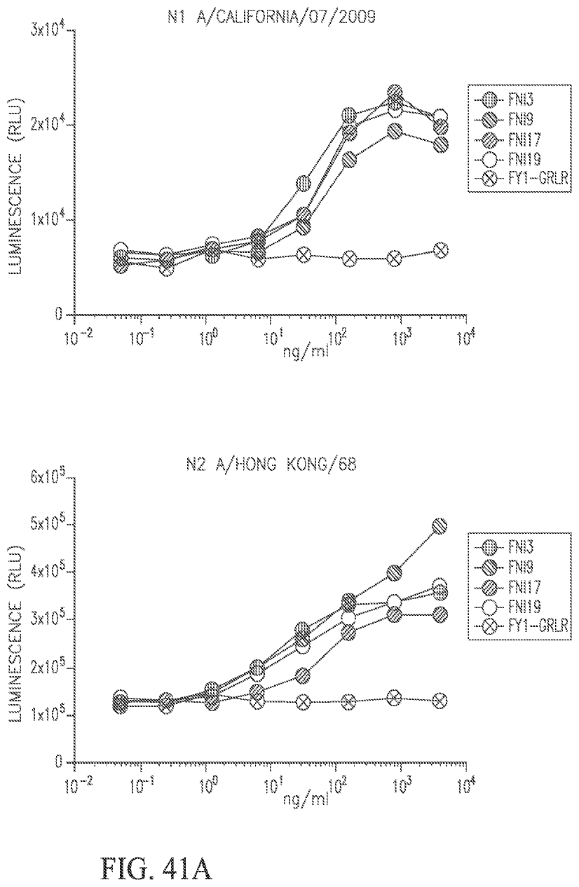

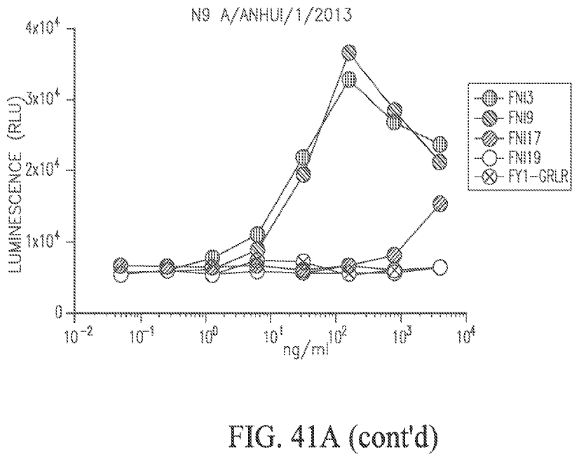

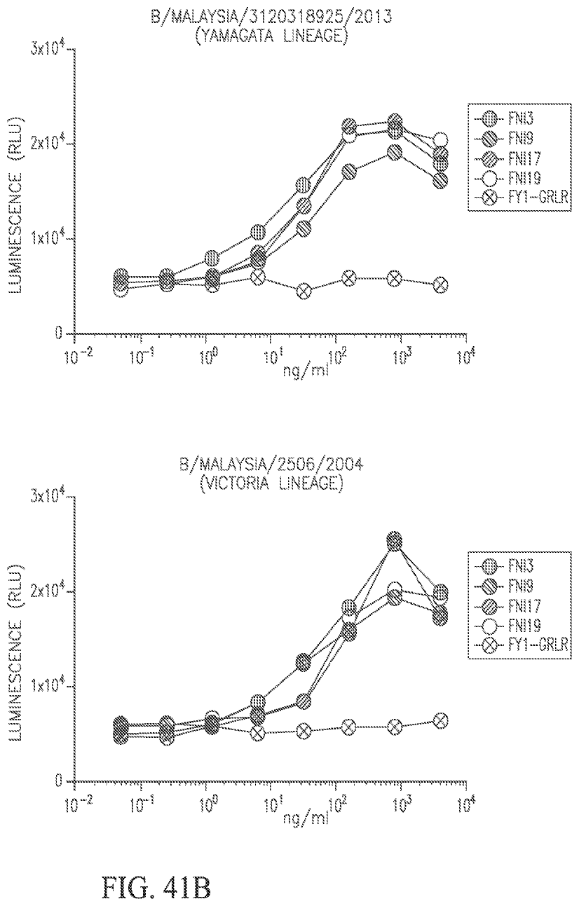

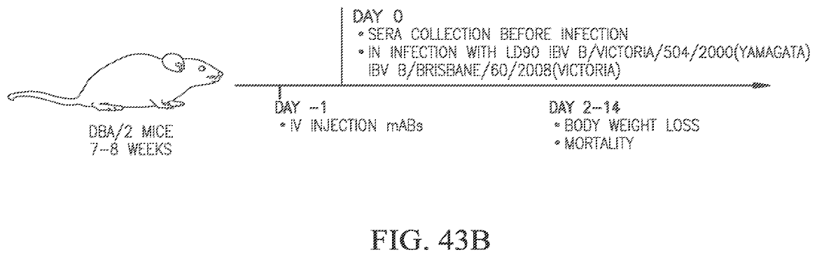

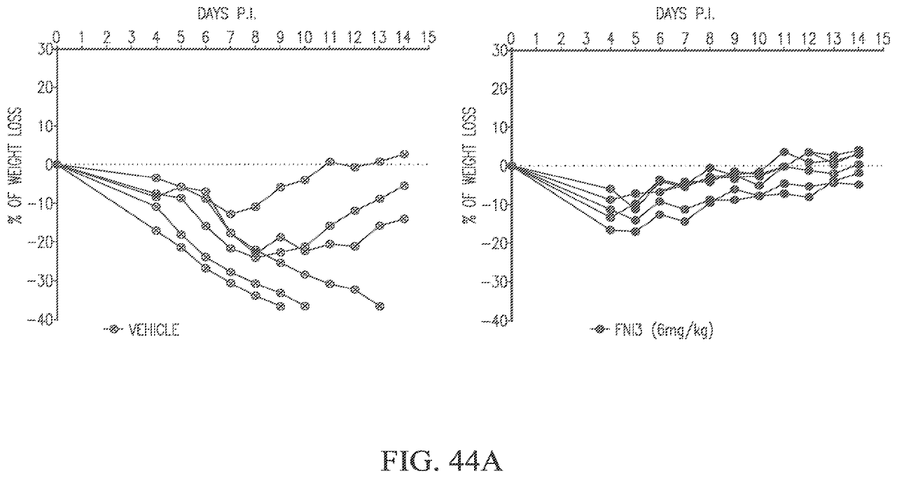

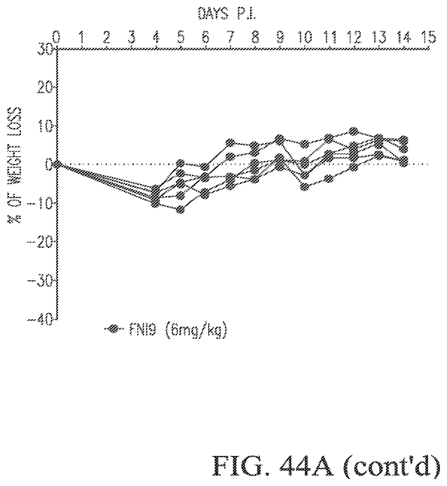

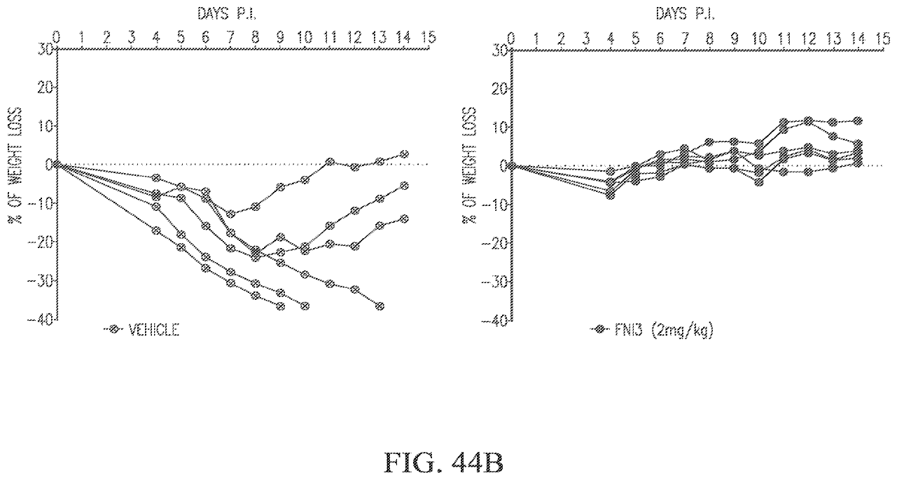

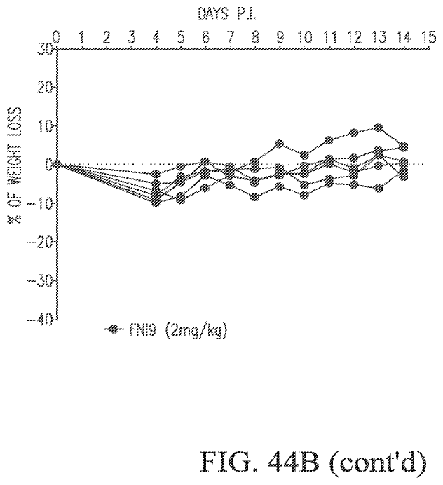

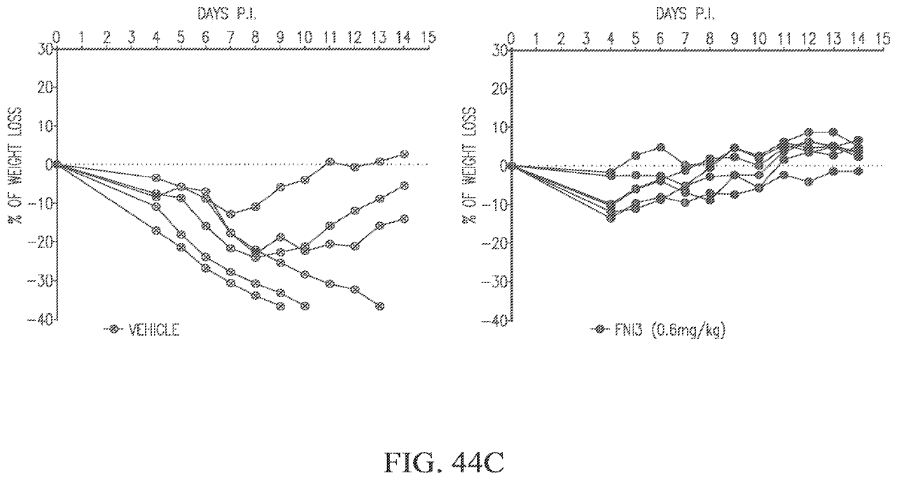

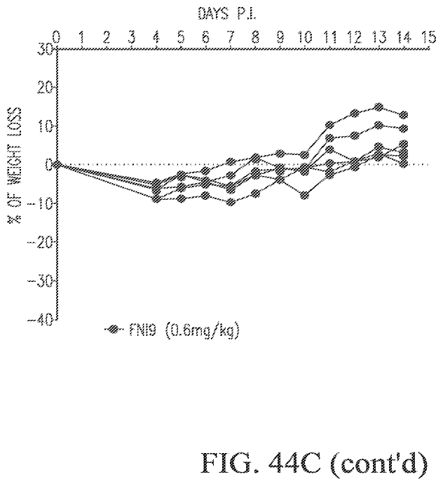

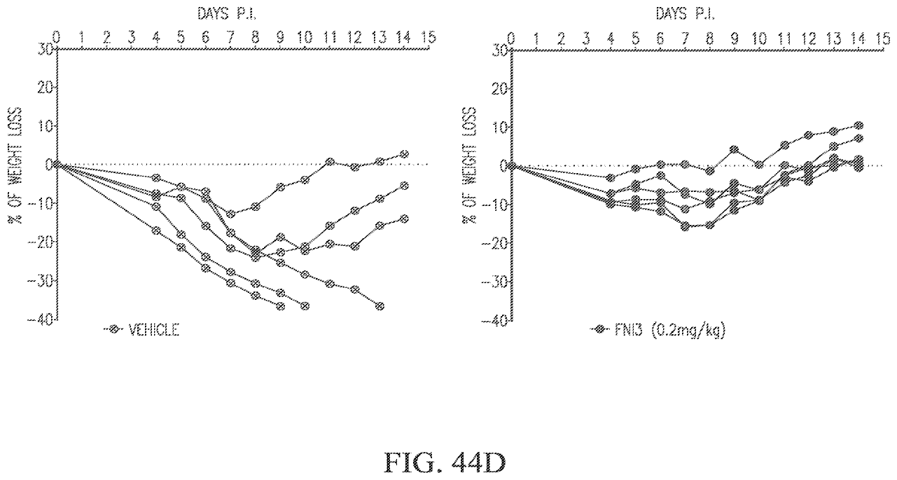

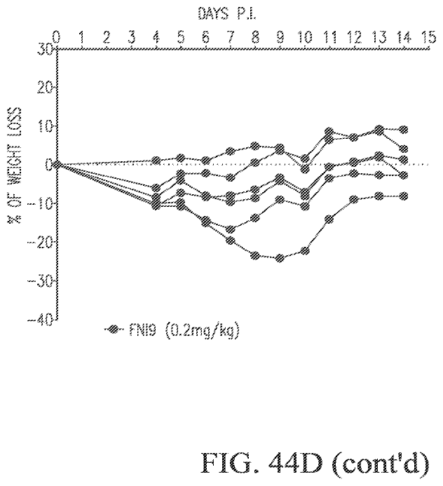

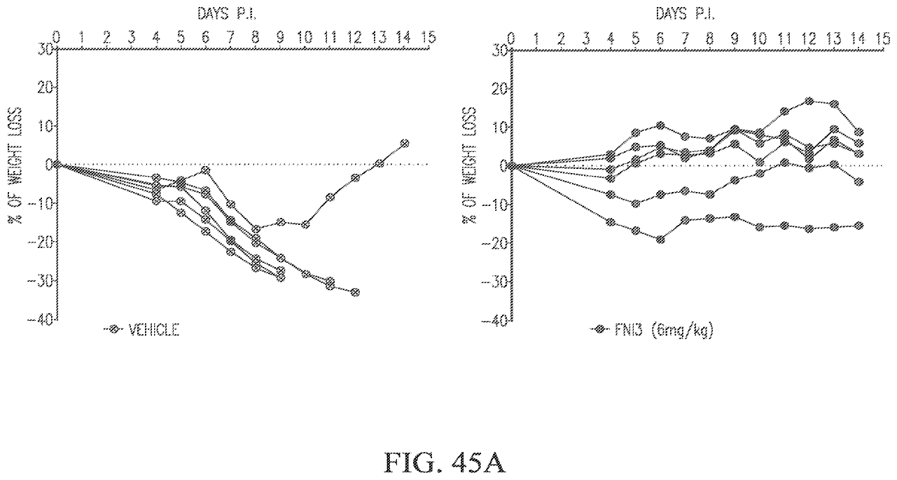

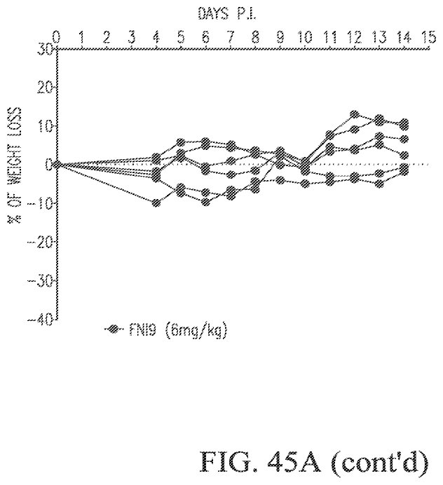

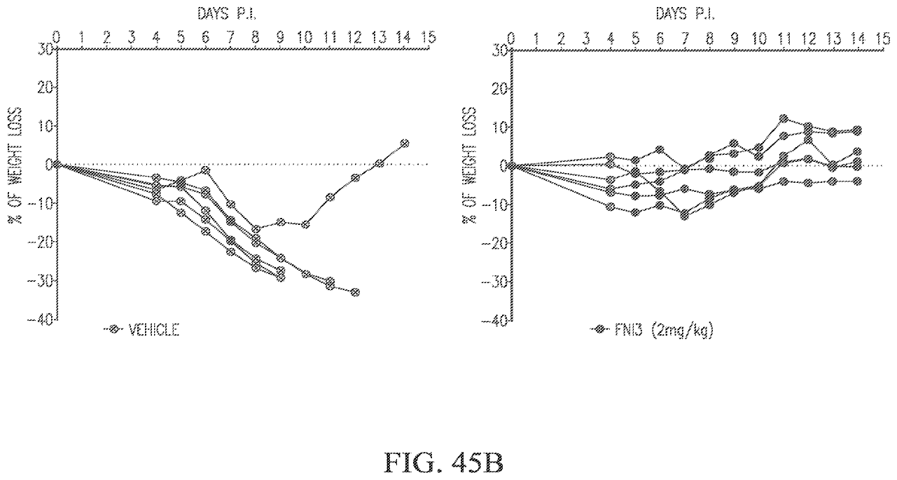

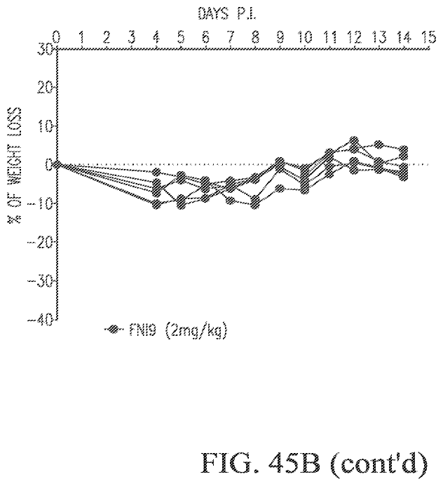

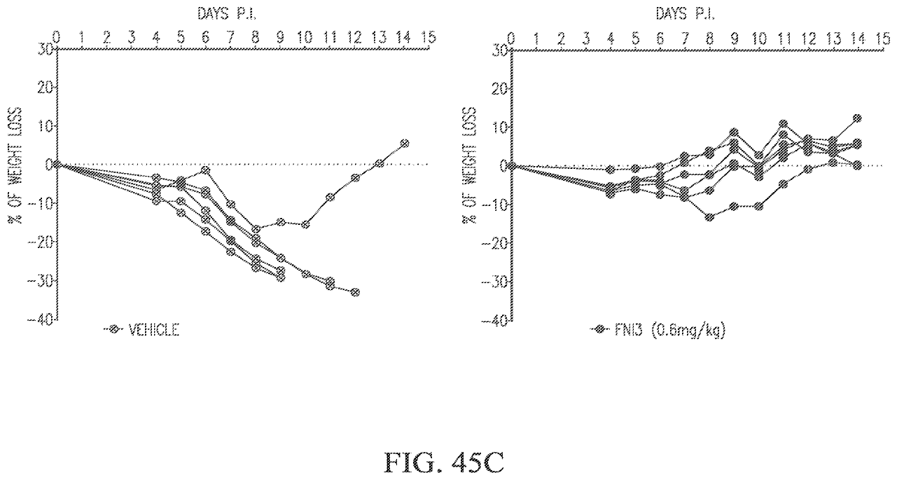

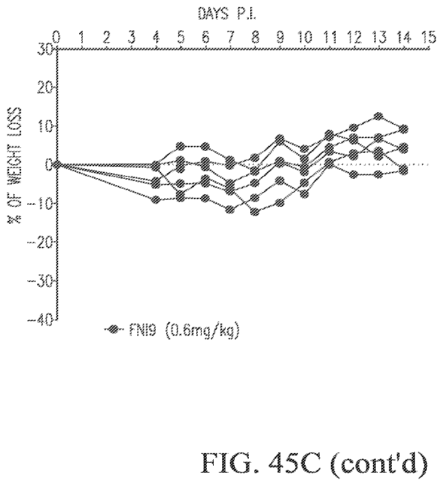

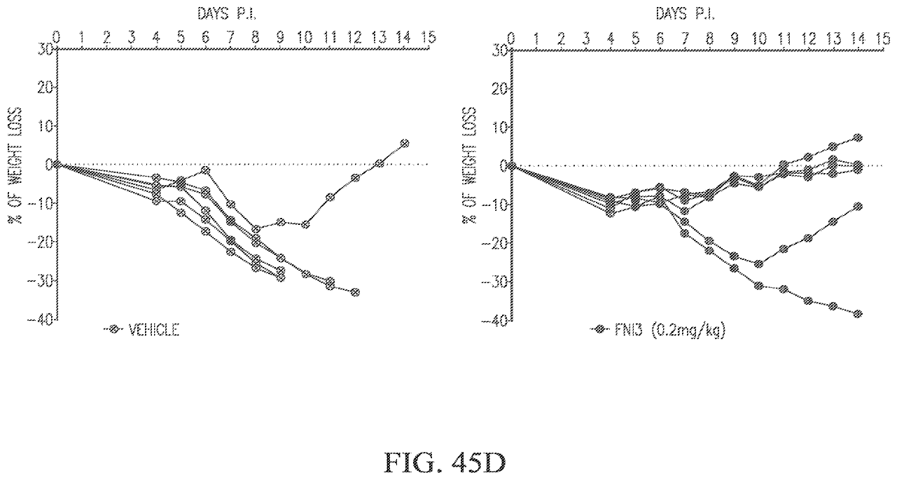

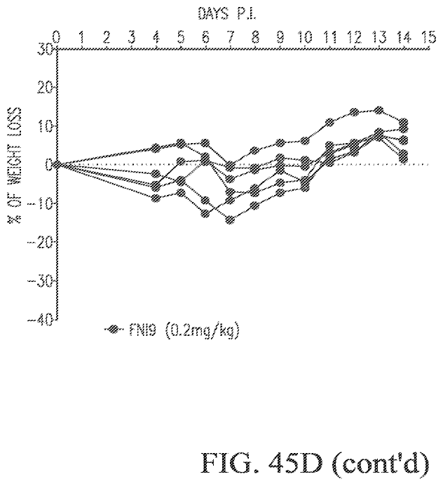

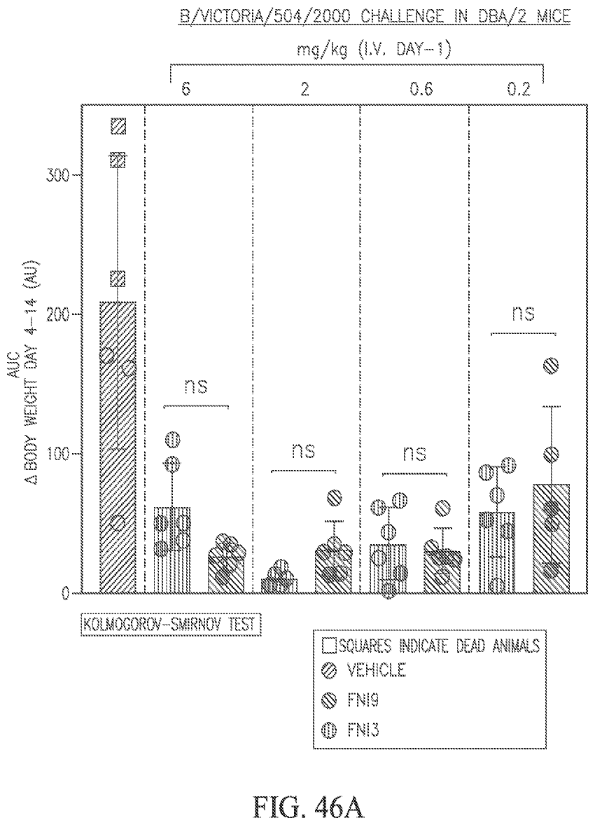

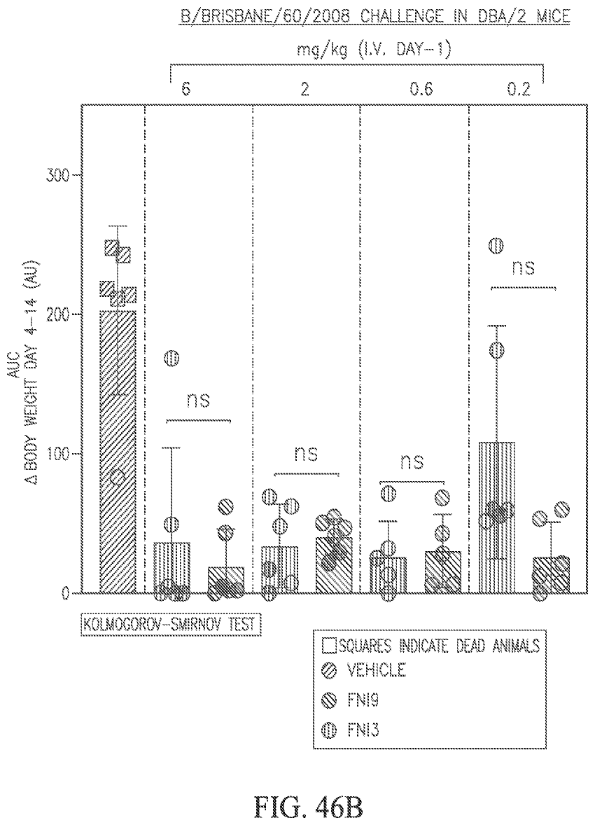

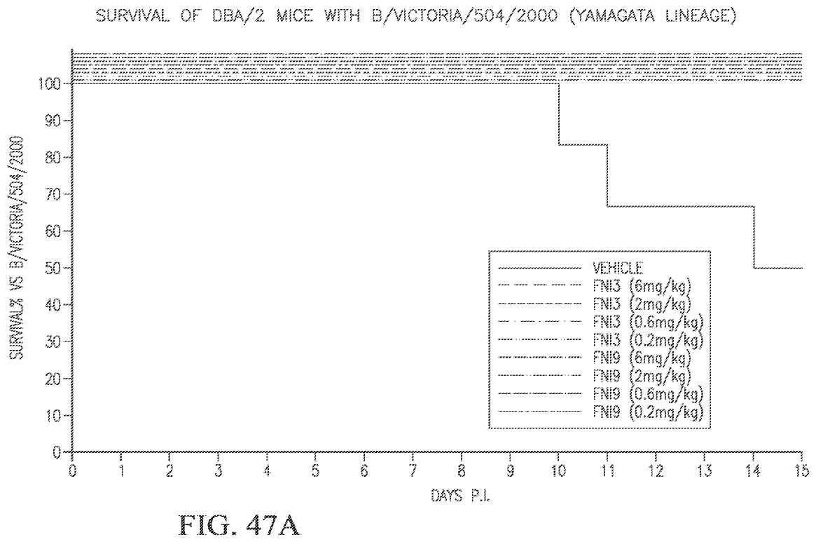

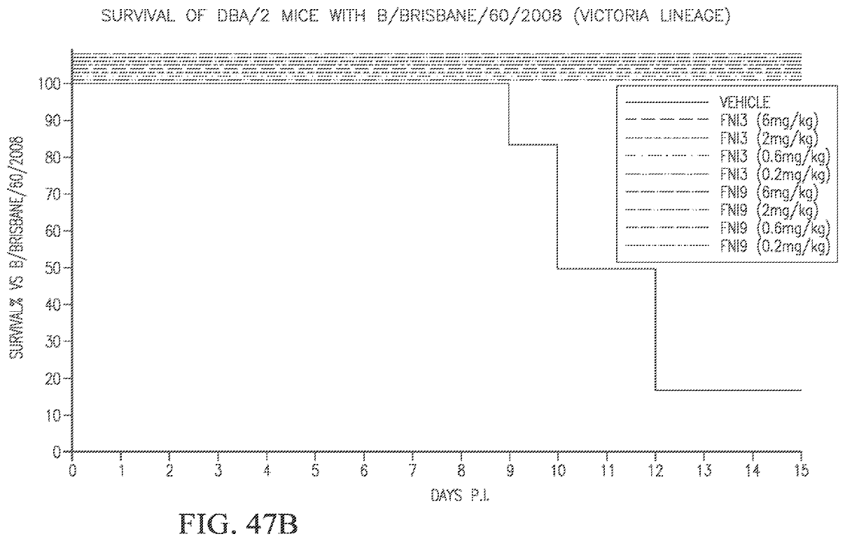

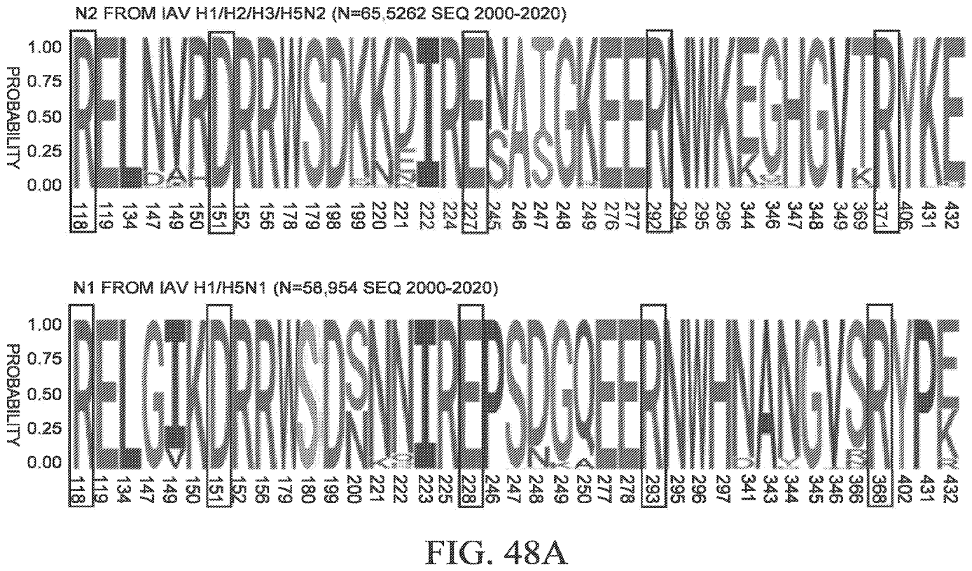

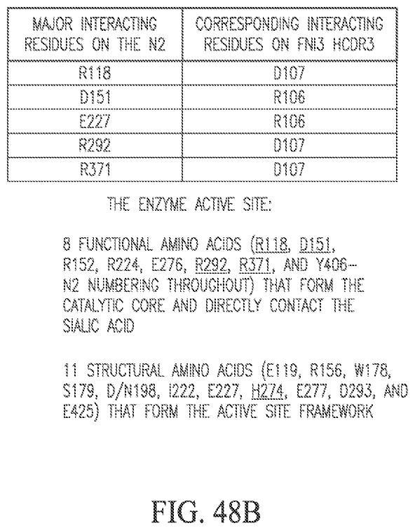

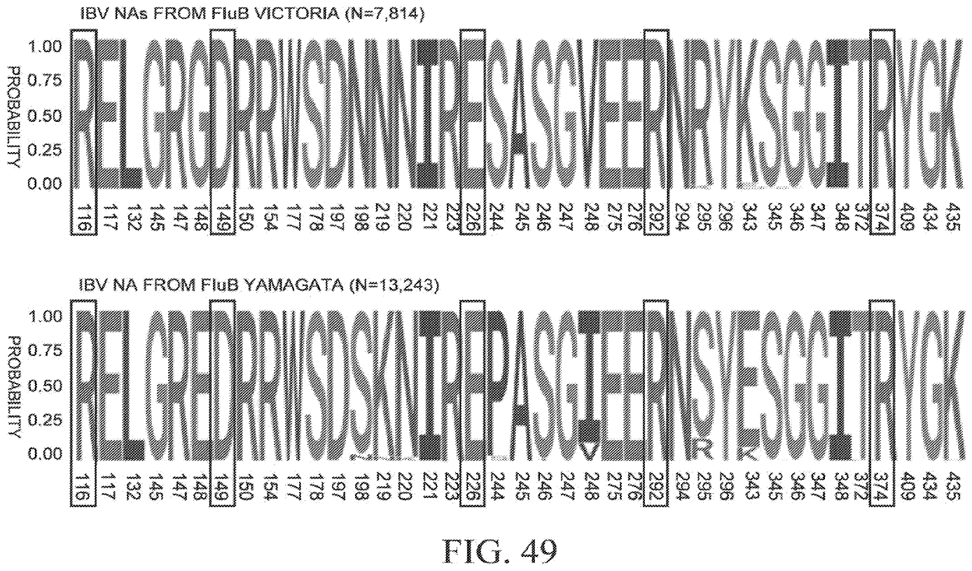

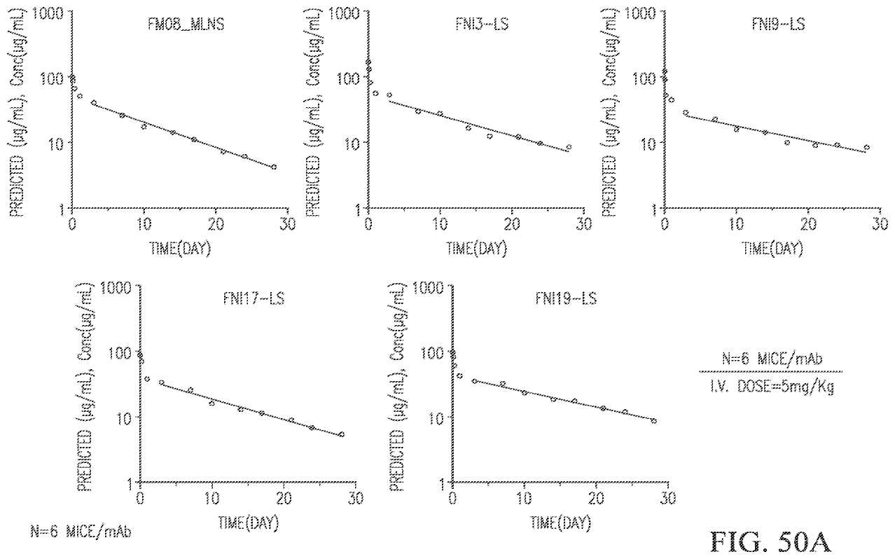

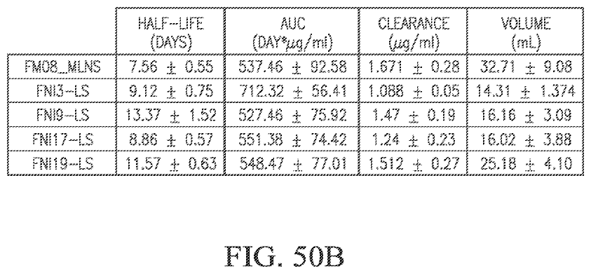

FNI antibodies were evaluated for binding and NAI activity against a panel of IAV NAs and IBV NAs (FIG. 33). FNI17 and FNI19 bound NA from human IAV circulating strains (e.g. N1 from A/California/07/2009 or N2 from A/Washington/01/2007) at a lower concentration than FNI3 and FNI9 (see data highlighted by rectangle in FIG. 33). FNI3 and FNI9 displayed higher cross-reactivity toward NAs from zoonotic strains (e.g. N9 from A/Anhui/1/2013, see data highlighted by rectangle in FIG. 33). All FNI antibodies bound to N1 from A/Swine/Jiangsu/J004/2018 (see data highlighted by rectangle, second from top in FIG. 33) which has been characterized as having pandemic potential (Sun et al. Proc Natl Acad Sci USA. 2020). The FNI sequence variants were analyzed for function. FNI antibodies were tested in further neutralization and NAI studies against IAVs and viruses bearing OSE-resistant mutations. FNI antibodies were tested for activation of FcγRs following incubation with IAV and IBV NAs. Epitope conservation studies and in vitro resistance selection studies were performed. In vivo prophylaxis studies of FNI3 and FNI9 against IAVs and against B/Victoria/504/2000 and B/Brisbane/60/2008 were performed in Balb/c and DBA/2 mice, respectively. In vivo pharmacokinetics of FNI antibodies bearing MLNS Fc mutations was tested in SCID Tg32 mice. Data from the above-mentioned studies are shown in FIGS. 33-50B. FNI9 showed a lack of polyreactivity against HEP-2 cells (data not shown).

Further studies (cryo-EM, resistance vs. H1N1 A/California/07/2009, PK in NHP, efficacy) are performed using FNI9, FNI7, and FNI19.

### Example 7

Conservation within the FNI NA Epitope

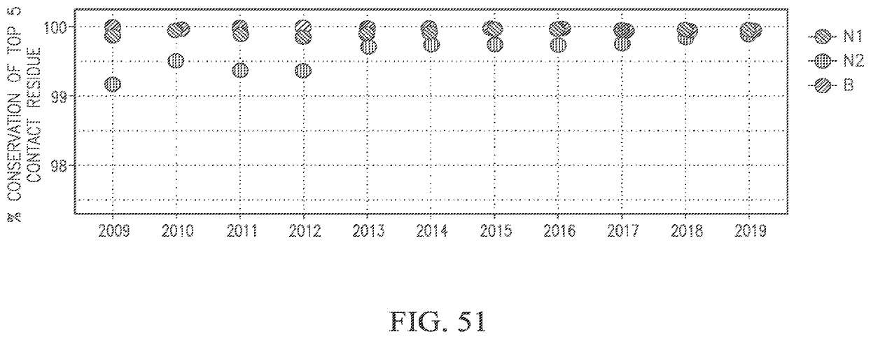

Binding interactions between anti-NA antibodies (FNI3, FNI17, and FNI19) and NA were evaluated by crystal structure studies and docking analysis. Conservation of the top five interacting residues within the FNI NA epitope in group I IAVs, group II IAVs, and IBVs from 2009 to 2019 are shown in FIG. 51.

### Example 8

In Vitro Potency: Comparison of FNI Antibodies with FM08 and Oseltamivir

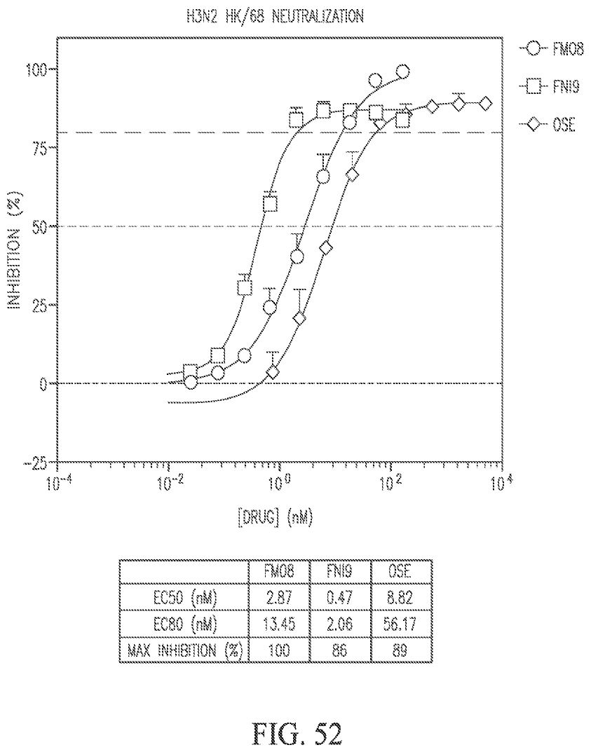

In vitro potency of FNI antibodies was evaluated in comparison with potency of OSE and FM08. In vitro neutralizing activity of FNI9, OSE, and a comparator antibody “FM08”, measured by nucleoprotein (NP) staining against H3N2 A/Hong Kong/8/68 virus, is shown in FIG. 52.

### Example 9

In Vivo Potency: Comparison of FNI Antibodies with FM08 and Oseltamivir

In vivo potency of FNI antibodies was evaluated in comparison with potency of OSE and FM08.

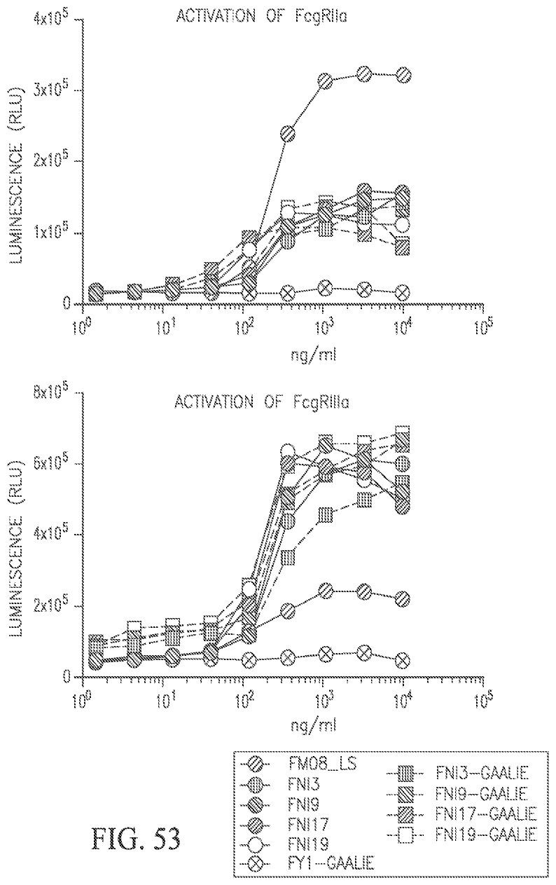

Antibody activation of FcγRIIIa and FcγRIIa by “GAALIE” variant antibodies (G236A/A330L/I332E variants) was tested, as shown in FIG. 53. Activation of FcγRIIIa (F158 allele) and FcγRIIa (H131 allele) was measured using an NFAT-mediated Luciferase reporter in engineered Jurkat cells. Activation was assessed following incubation with A549 cells infected with H1N1 influenza strain A/Puerto Rico/8/34 at a multiplicity of infection (MOI) of 6. FNI3, FNI9, FNI17, and FNI19 were tested, along with FNI3, FNI9, FNI17, and FNI19 antibodies bearing GAALIE mutations (suffix “-GAALIE”). A comparator antibody “FM08_LS” and a negative control antibody (FY1-GRLR) were also tested.

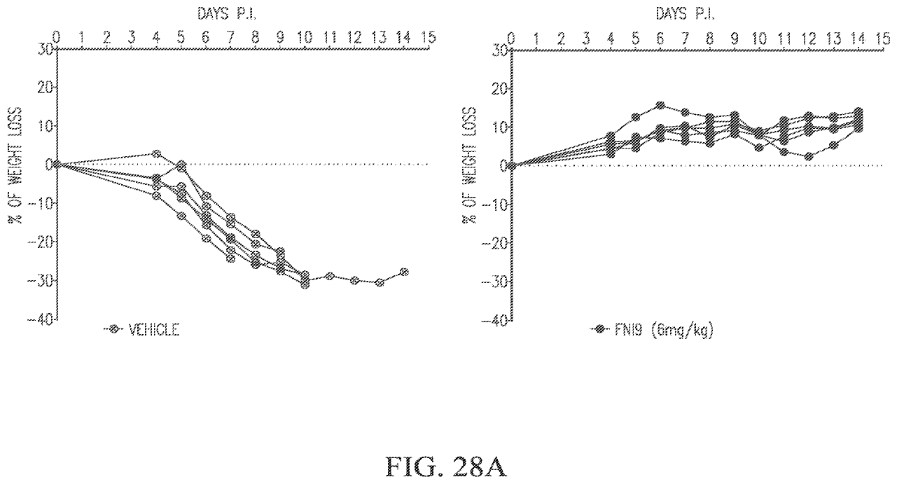

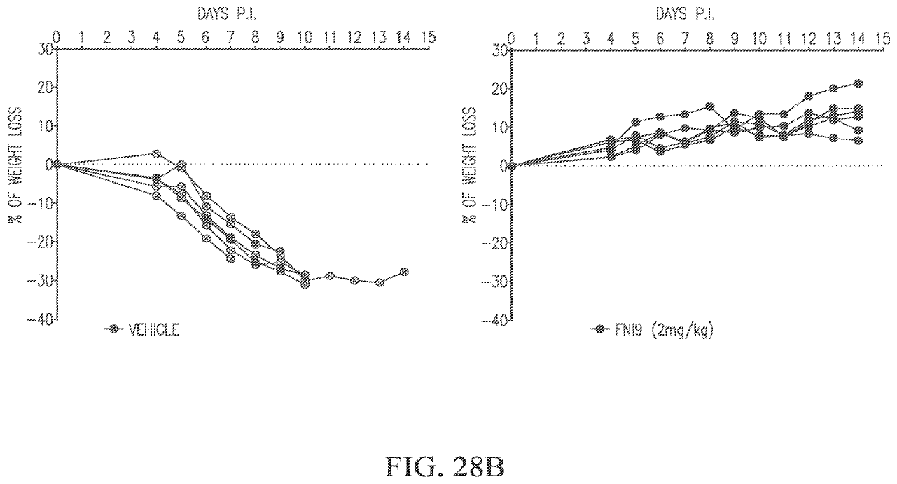

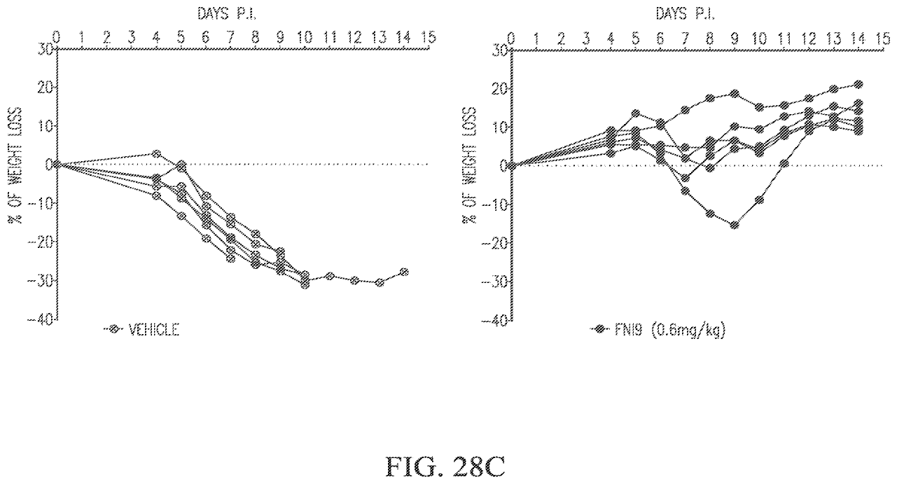

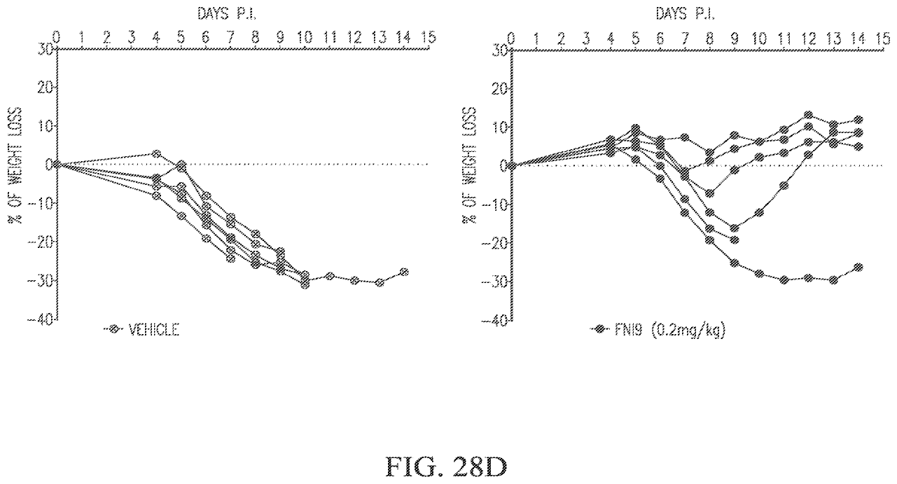

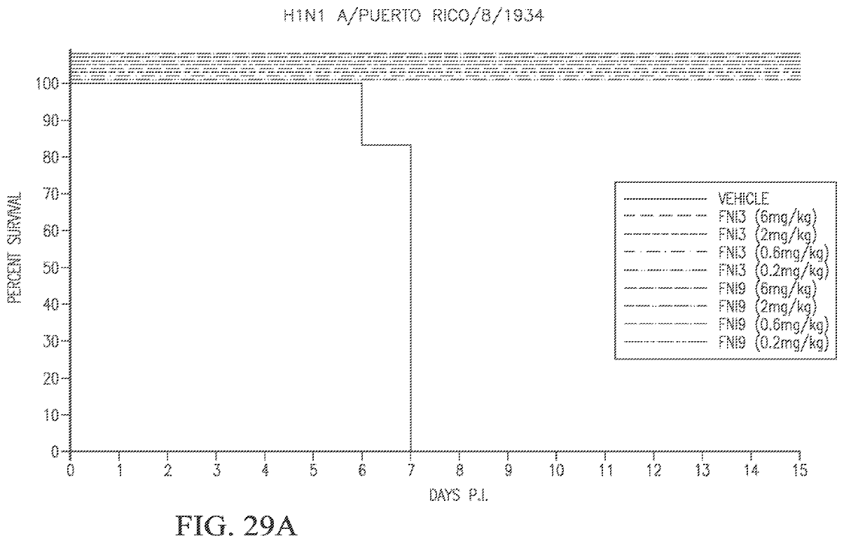

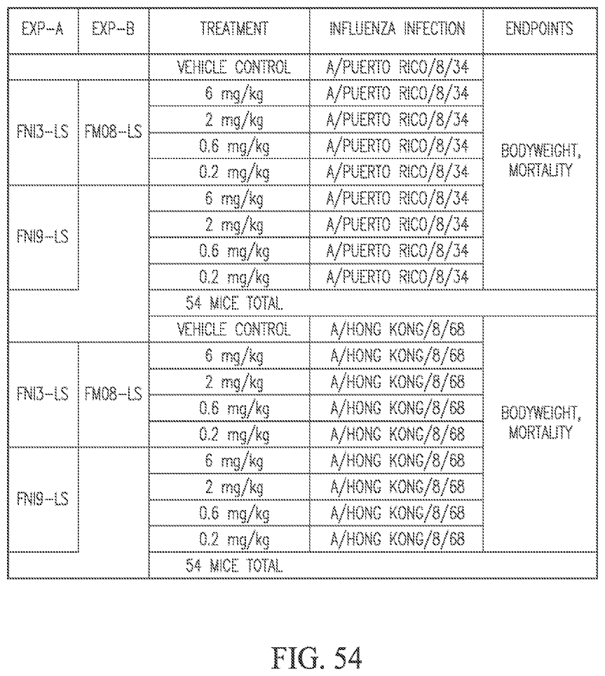

An inter-experiment in vivo study was designed to compare prophylactic activity of FM08_LS with FNI3 and FNI9 in BALB/c mice infected with H1N1 IAV A/Puerto Rico/8/34 or H3N2 IAV A/Hong Kong/8/68 (FIG. 54). Antibody was administered at 6 mg/kg, 2 mg/kg, 0.6 mg/kg, or 0.2 mg/kg, one day prior to infection with a LD90 (90% lethal dose) of A/Puerto Rico/8/34 or H3N2 IAV A/Hong Kong/8/68. The timeline, data collection, and endpoints of the study are the same as those seen in FIG. 26B. In Experiment A (“Exp-A”) BALB/c mice were infected with A/Puerto Rico/8/34 following pre-treatment with FNI3 (data not shown) or FNI9 (FIGS. 27A-27D). In another arm of Experiment A, BALB/c mice were infected with A/Hong Kong/8/68 following pre-treatment with FNI3 (data not shown) or FNI9 (FIGS. 28A-28D). In Experiment B (“Exp-B”) BALB/c mice were infected with A/Puerto Rico/8/34 (data not shown) or A/Hong Kong/8/68 (data not shown) following pre-treatment with FM08_LS.

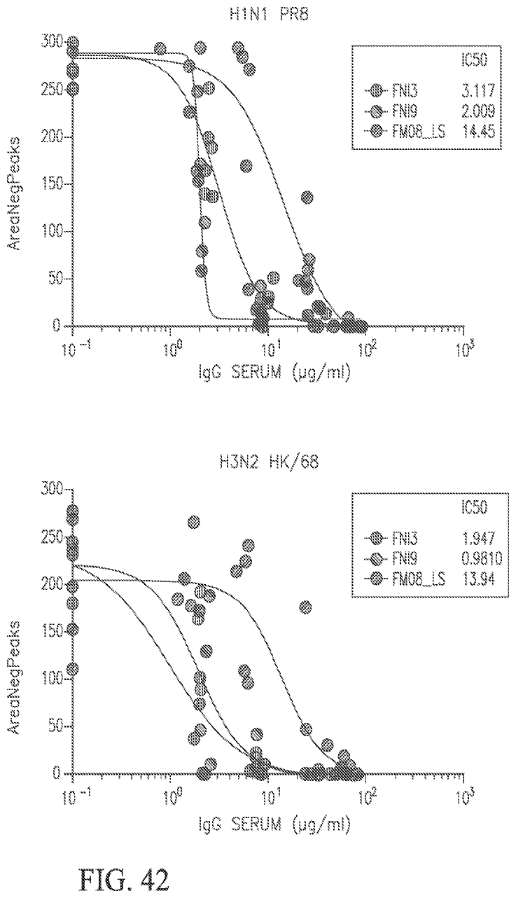

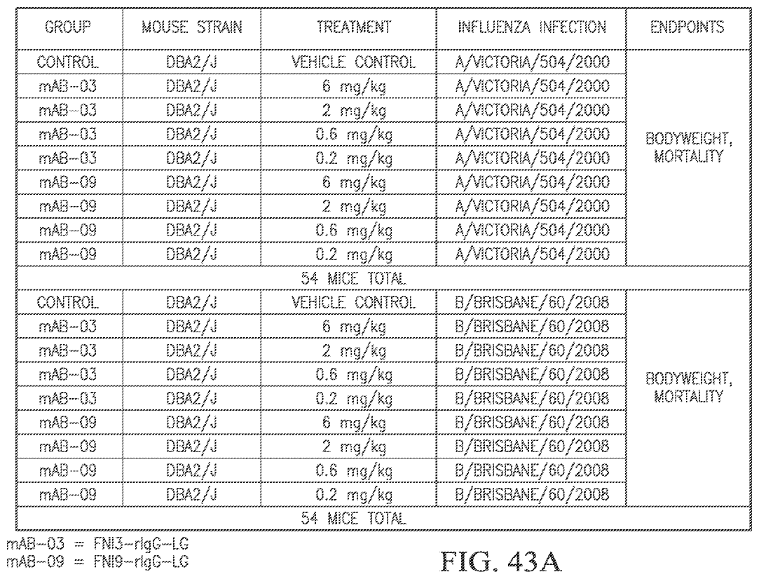

Body weight measurements for the FNI9 test group over fifteen days are shown in FIGS. 27A-27D (A/Puerto Rico/8/34 FNI9 test group) and 28A-28D (A/Hong Kong/1/68 FNI9 test group). Negative area-under-the-curve peak values compared with IgG in serum from area-under-the-curve analysis of body weight loss in BALB/c mice infected with A/Puerto Rico/8/34 (H1N1) or A/Hong Kong/8/68 (H3N2) following treatment with FNI3 or, FNI9, or FM08_LS are shown in FIG. 42.

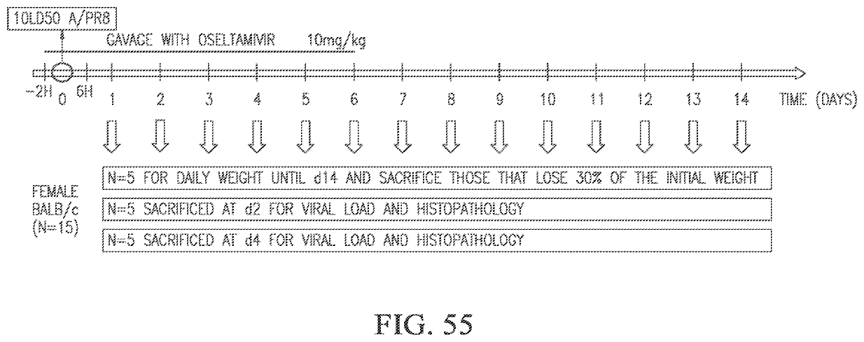

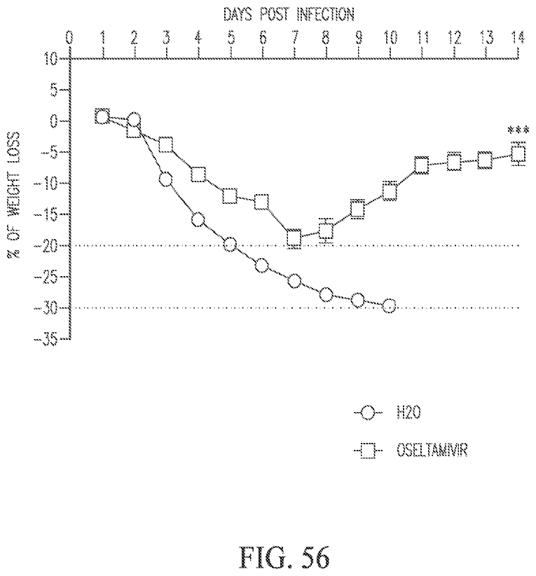

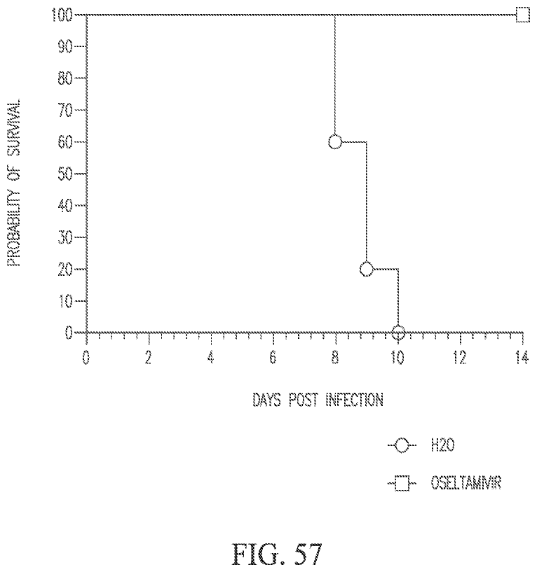

An in vivo study was designed to evaluate biological potency of oseltamivir (OSE) in female BALB/c mice infected with IAV A/Puerto Rico/8/34 (FIG. 55). OSE was administered at 10 mg/kg by oral gavage on Day 0 beginning at two hours prior to infection with 10-fold LD50 (50% lethal dose) of A/Puerto Rico/8/34. OSE was administered at the same dose at 6 hours post-infection and then twice daily until day 6 post-infection. Body weight measurements over fourteen days are shown in FIG. 56 and survival over fourteen days is shown in FIG. 57.

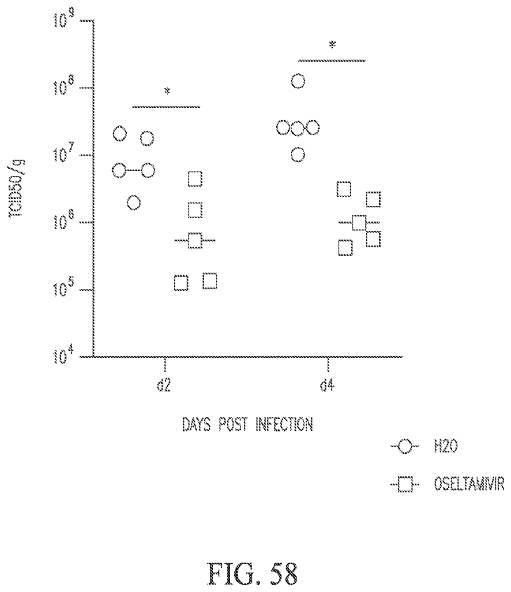

Viral titers in lung homogenates from OSE-treated mice were measured from samples obtained at two and four days post-infection (FIG. 58).

### Example 10

### Generation and Characterization of FNI3, FNI9, FNI17, and FNI19 Variant Antibodies

Variable domain sequence variants were generated from FNI3, FNI9, FNI17, and FNI19 and characterized for binding and neutralization. A total of thirty-two (32) variant antibodies were generated, in which twenty-six (26) variants contained a reversion of VH and/or VL framework amino acid(s) to germline sequence, three (3) FNI17 variants contained a reversion of VH framework regions to germline sequence and a W97A/L/Y mutation in VL, and three (3) FNI17 variants contained a wild-type VH and a W97A/L/Y mutation in VL. A total of 11 variants were generated from FNI3, 5 variants from FNI9, 11 variants from FNI17, and 5 variants from FNI19.

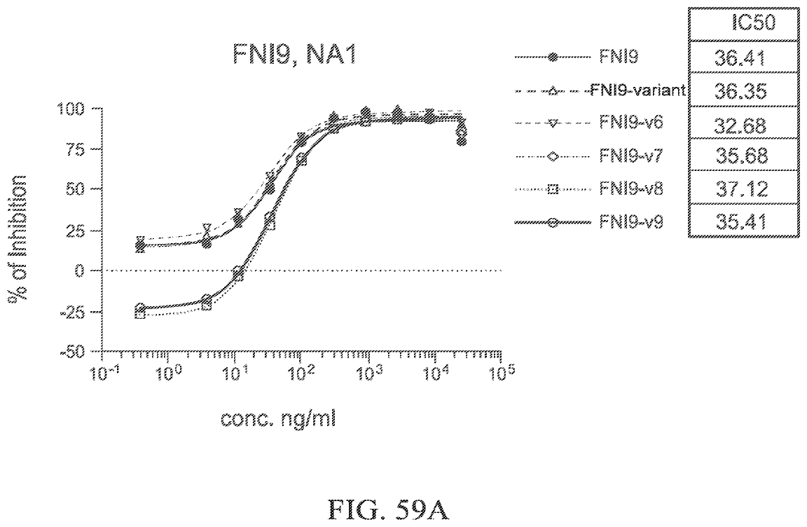

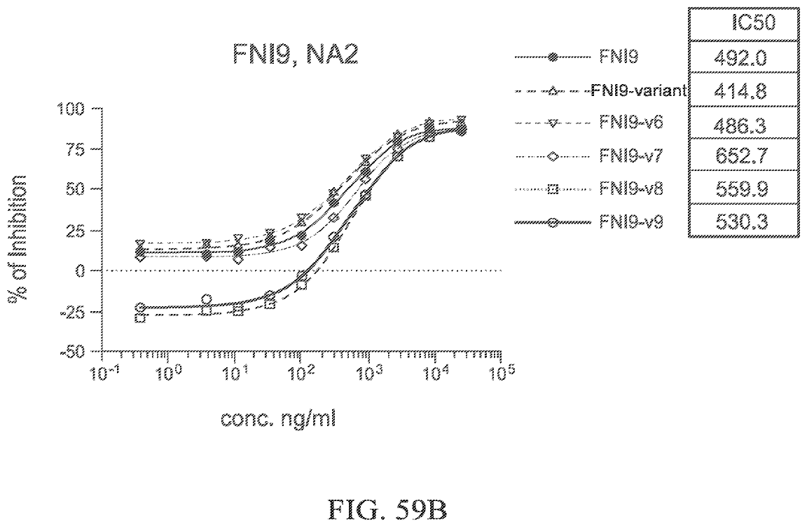

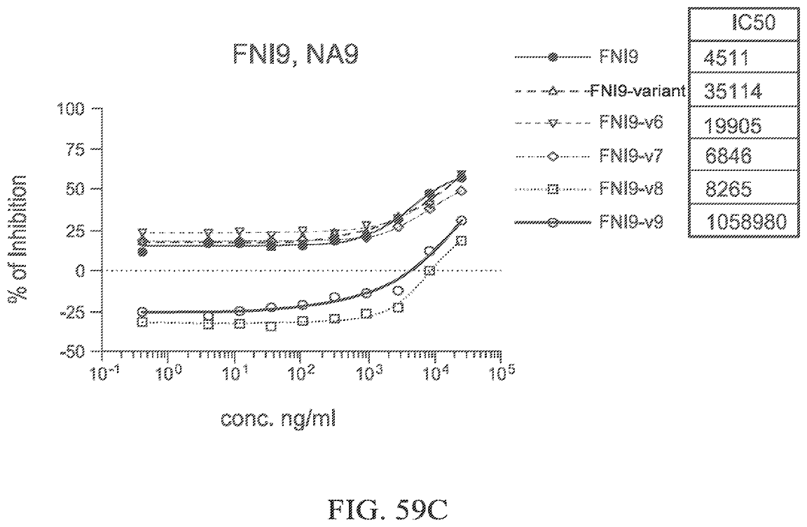

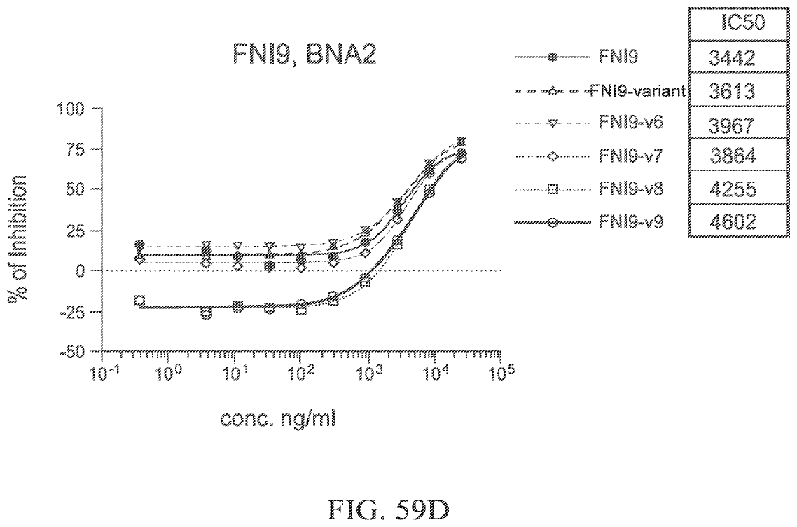

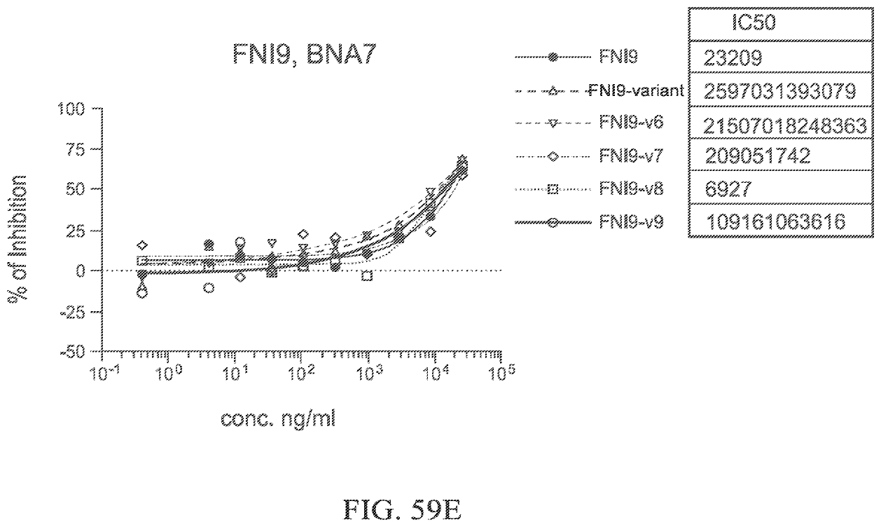

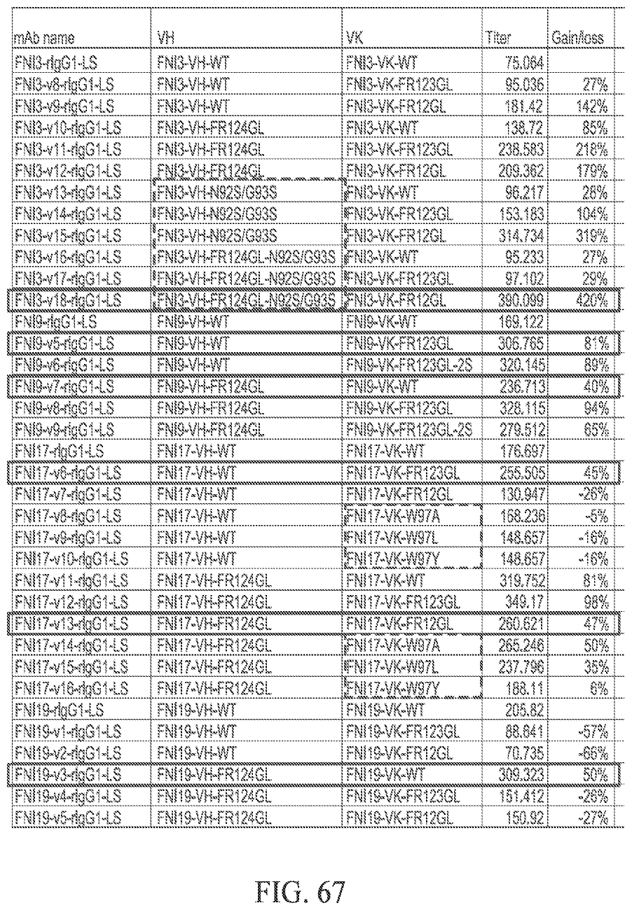

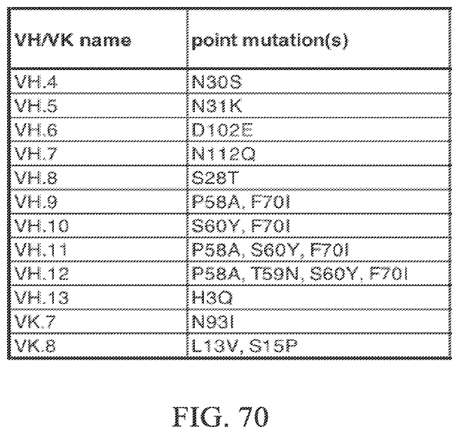

In vitro inhibition of sialidase activity against IAV NAs (NA1 from H5N1 A/Vietnam/1203/2004; NA2 from H3N2 A/Tanzania/205/2010; NA9 from H7N9 A/Hong Kong/56/2015) and IBV NAs (BNA7 from B/Malaysia/2506/2004; BNA2 from B/Perth/211/2011) by the developability variants was measured. Inhibition activity for FNI9 and variants “FNI9-variant” through “FNI9-v9” is shown in FIGS. 59A-59E). In these figures, “FNI9-v6”, “FNI9-v7”, “FNI9-v8”, and “FNI9-v9” are as shown in FIG. 67 and are not the same as the FNI9 variant sequences “-VH.6”, “-VH.7”, “-VH.8”, and “-VH.9” as shown in e.g. FIGS. 70-72.

Binding of all thirty-two (32) variants to IAV NAs and IBV NAs was evaluated by FACS to exclude potential loss of breadth due to reversion to germline of mAb framework regions. Results included positive binding of FNI9 and the generated FNI9 variants to N1 from A/Stockholm/18/2007, A/California/07/2009, and A/California/07/2009 I23R/H275Y; N2 from A/South Australia/34/2019, A/Leningrad/134/17/57, and A/Washington/01/2007; N3 from A/Canada/rv504/2004; N6 from A/swine/Ontario/01911/1/99; N7 from A/Netherlands/078/03; IBV NA from B/Yamanashi/166/1998 (Yamagata), B/Malaysia/2506/2004 (Victoria), and B/Lee/10/1940 (Ancestral).

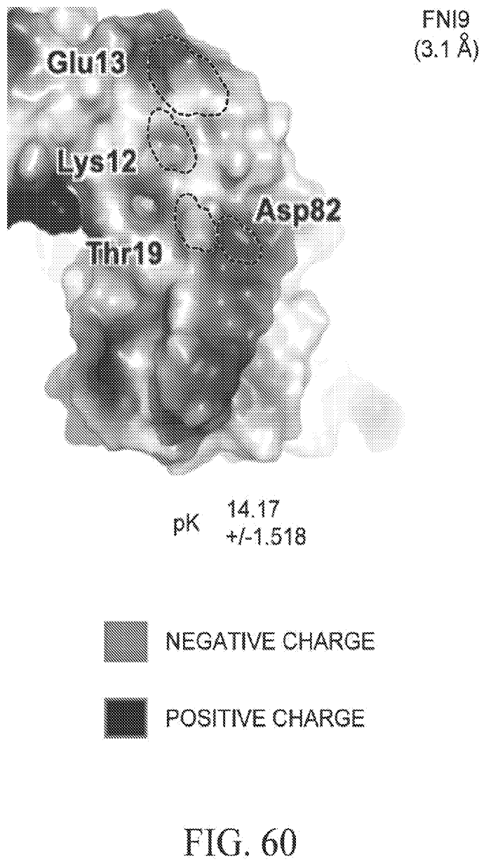

Surface charge and pharmacokinetic (pK) values were determined for FNI3, FNI9, FNI17, and FNI19. Overall surface charge maps were generated using PyMOL (The PyMOL Molecular Graphics System, Version 1.2r3pre, Schrödinger, LLC), data for FNI9 are provided (FIG. 60) along with pK values and resolution (reported in Å). Without being bound by theory, decreases in overall positive charge on the surface of the antibody may serve to reduce sequestration of the antibody by pinocytosis on the cell surface. FNI9 presented a more negative surface charge and a correspondingly improved pK value in comparison to FNI3, FNI17, and FNI19 (data not shown).

### Example 11

### Further Studies

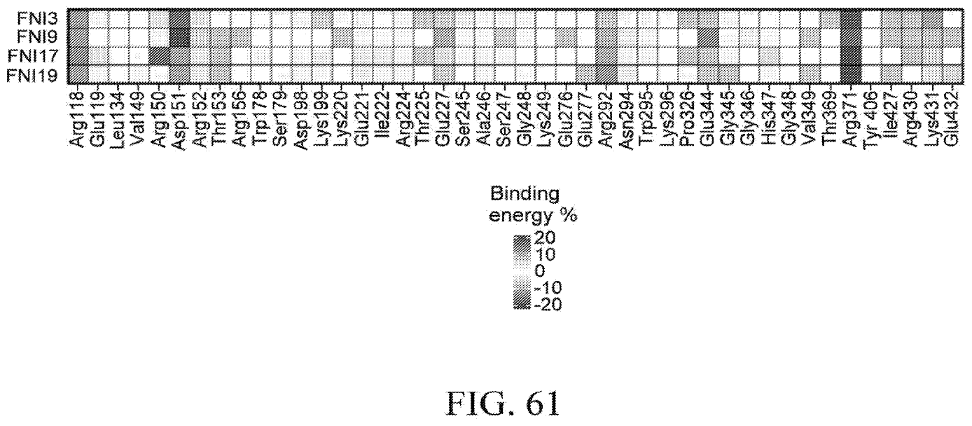

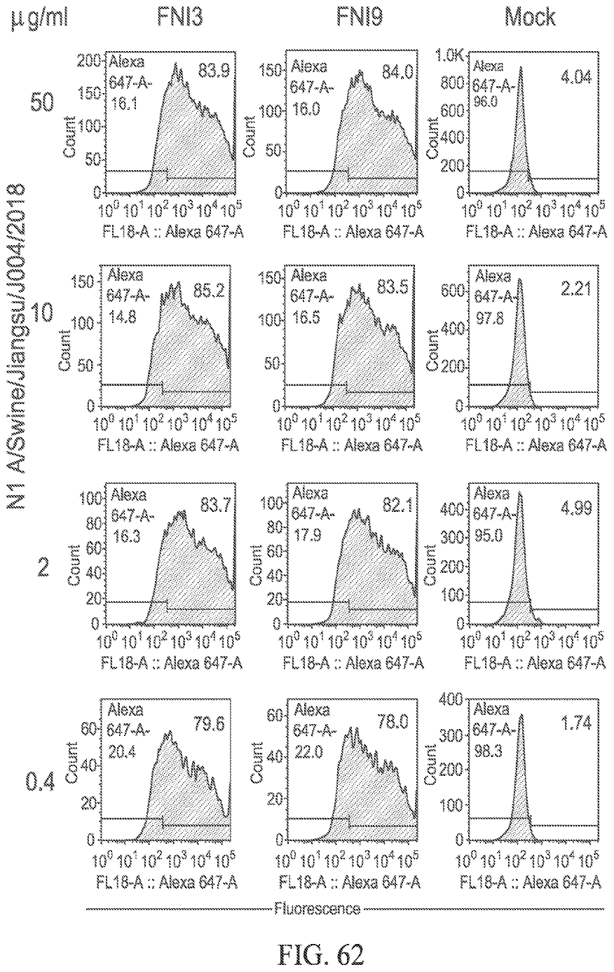

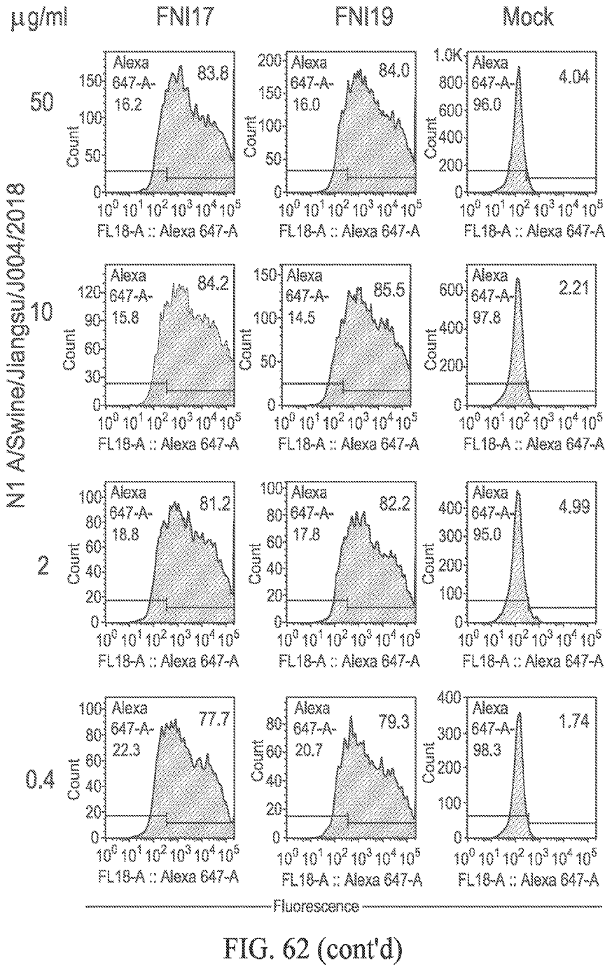

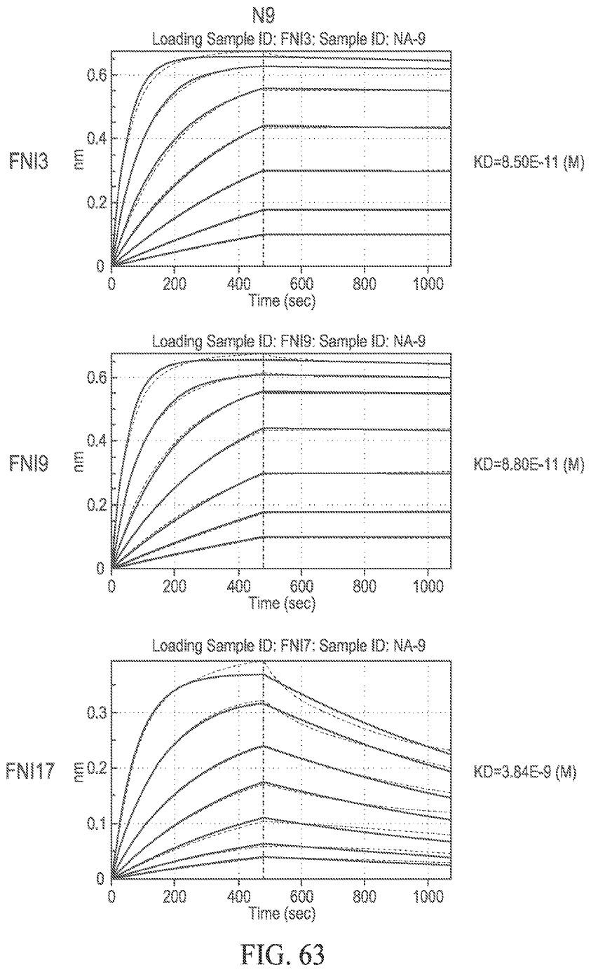

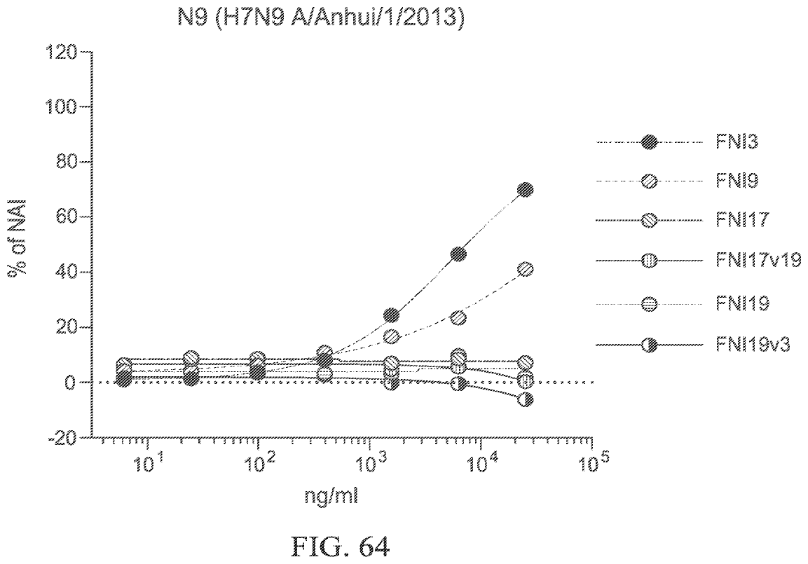

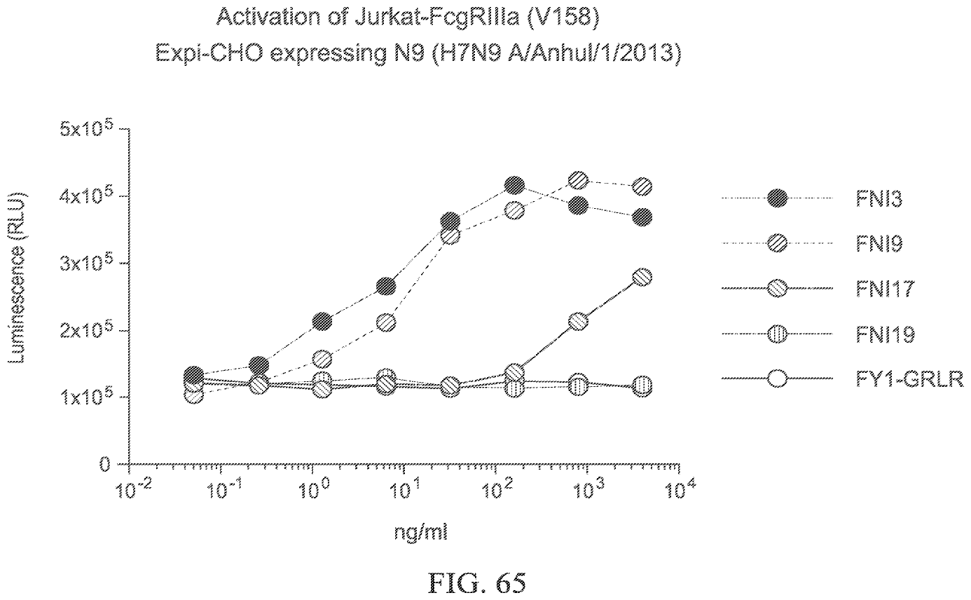

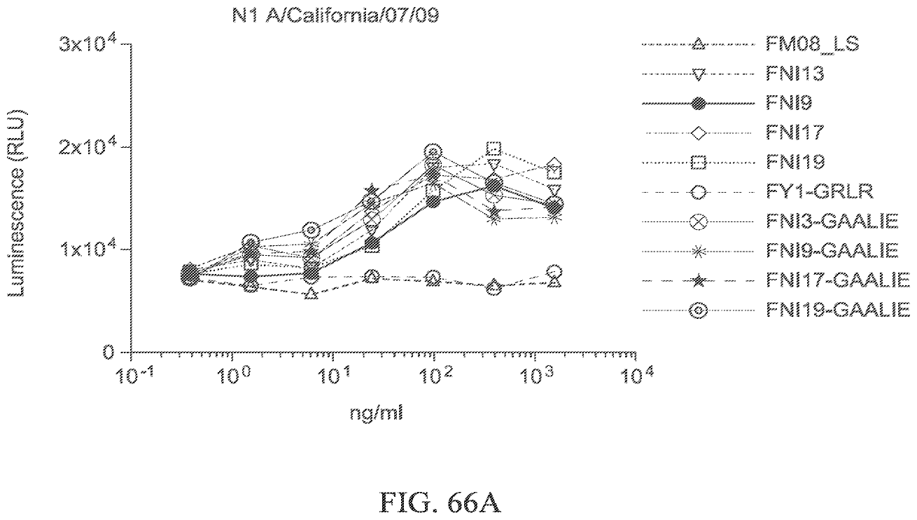

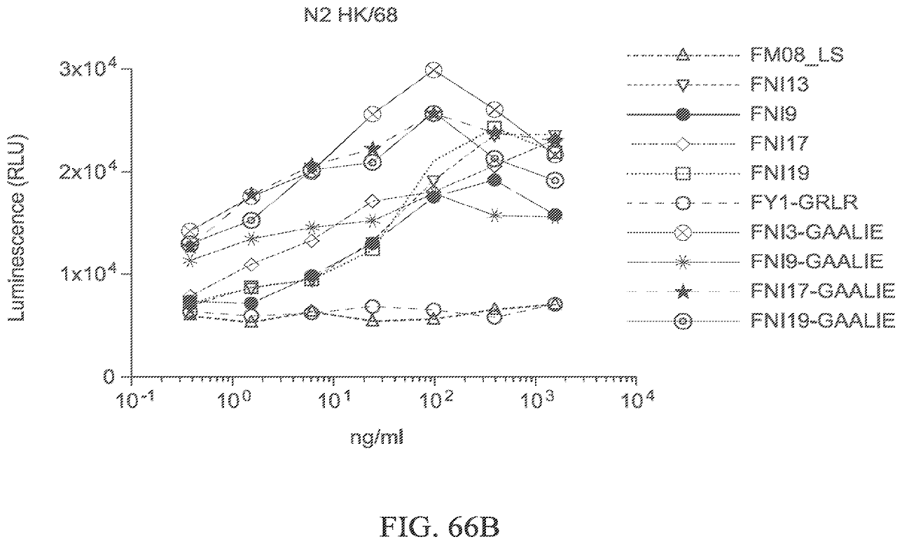

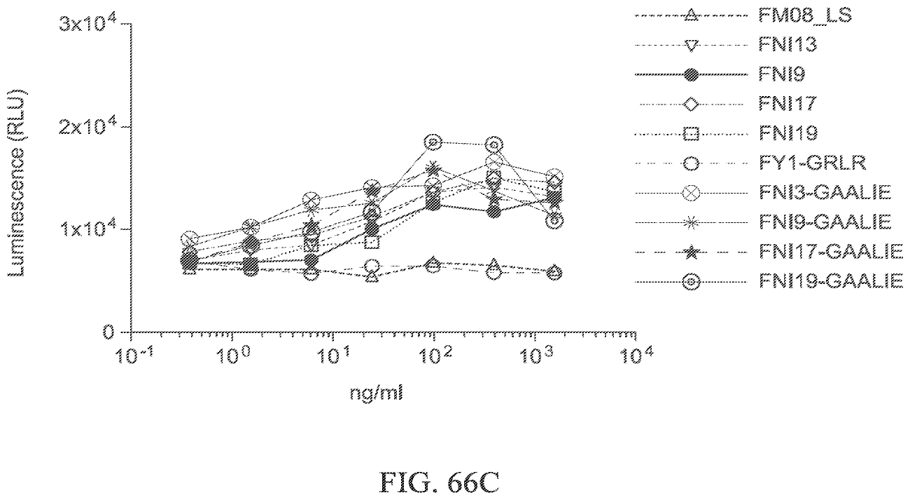

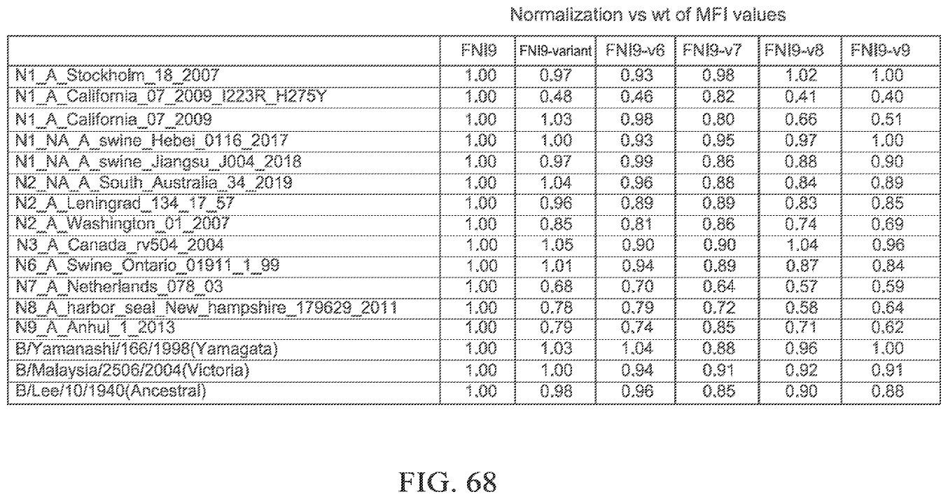

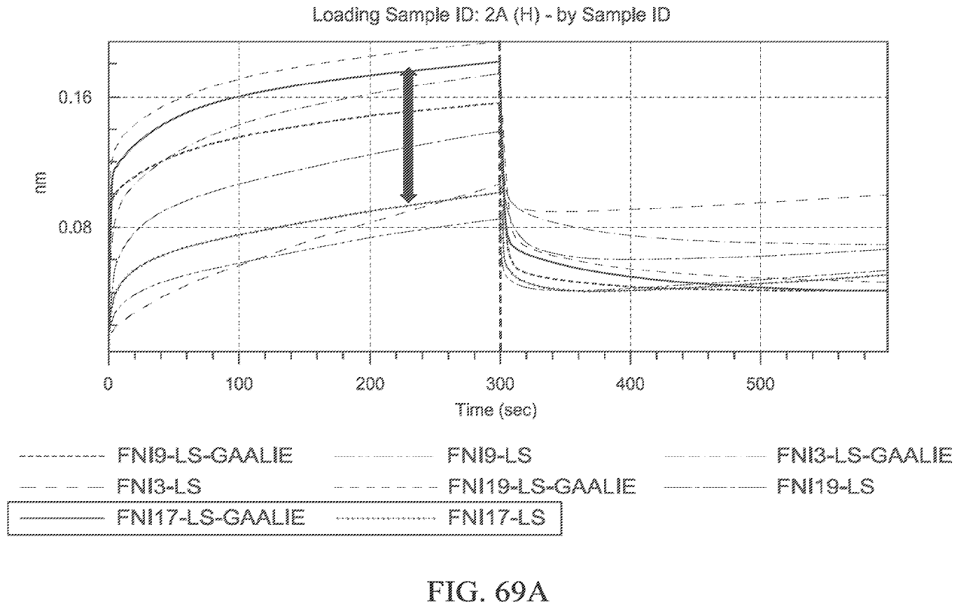

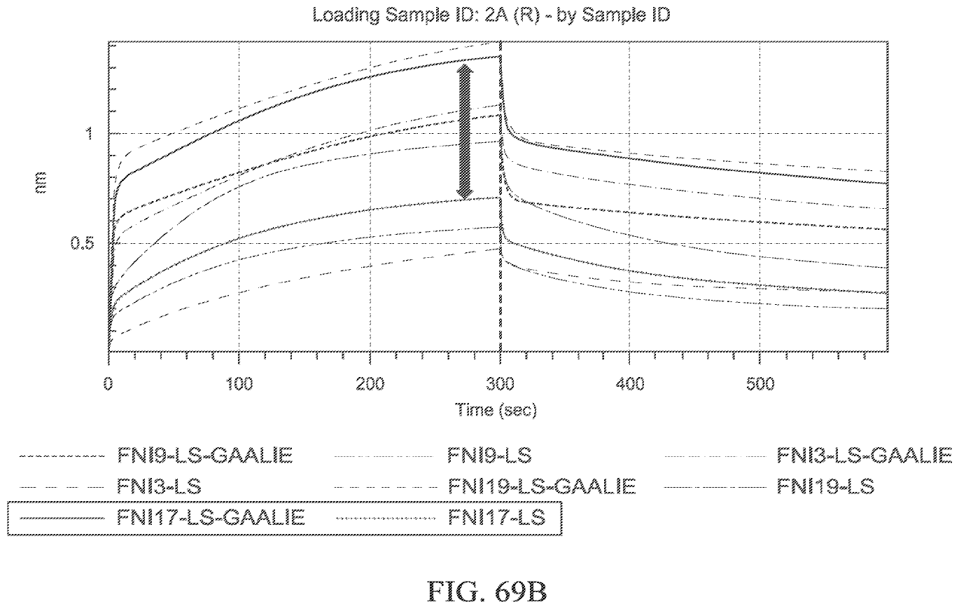

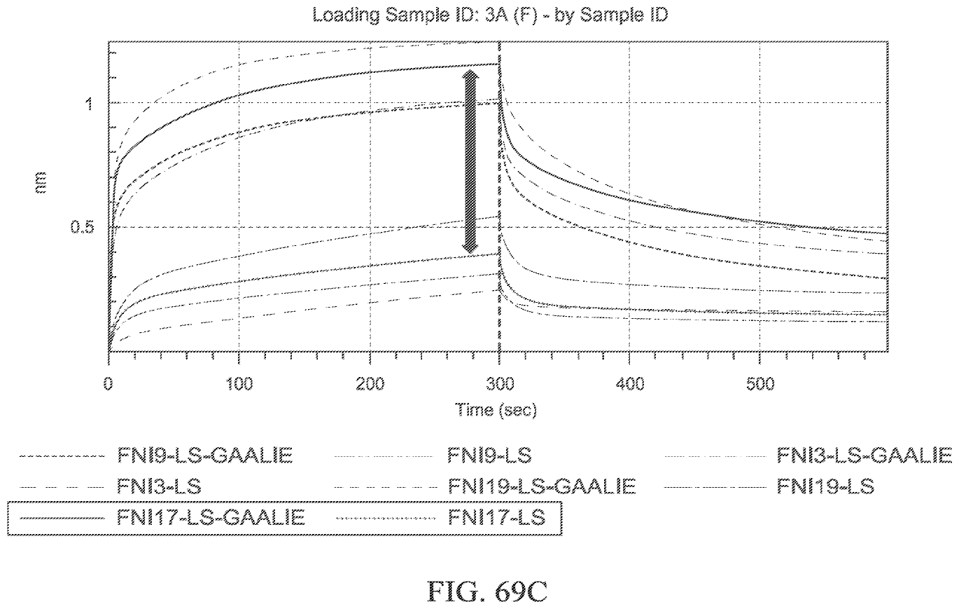

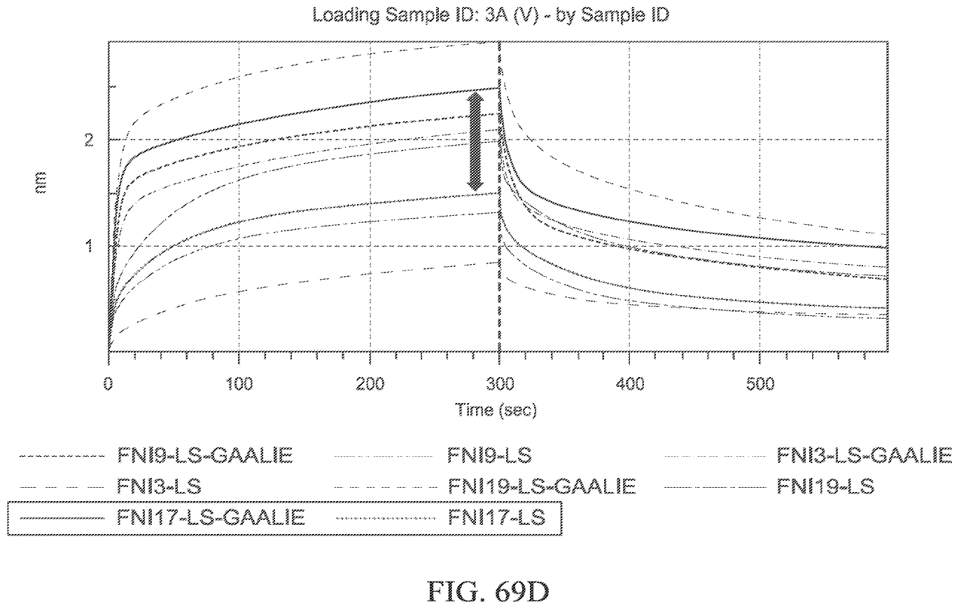

Additional studies were performed, as described and shown in FIGS. 61-69D.

### Example 12

### Testing of FNI9-v5

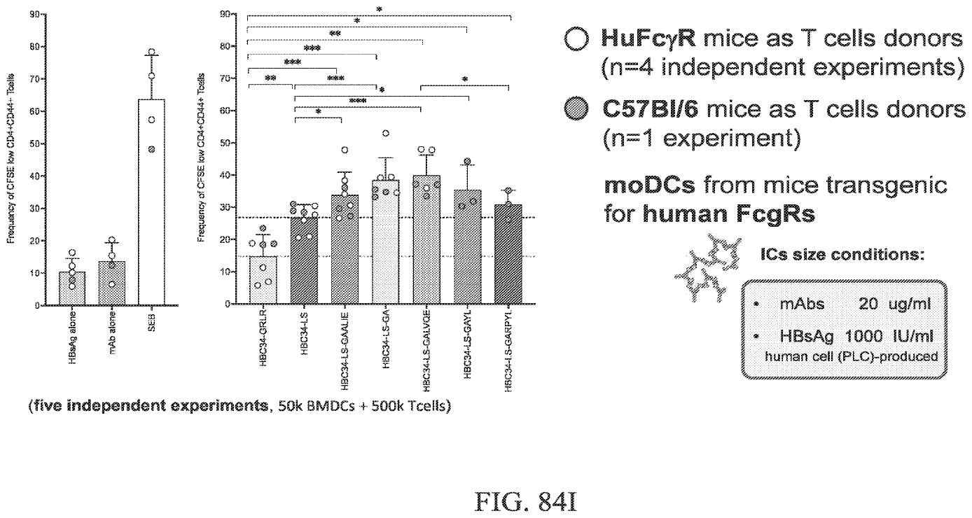

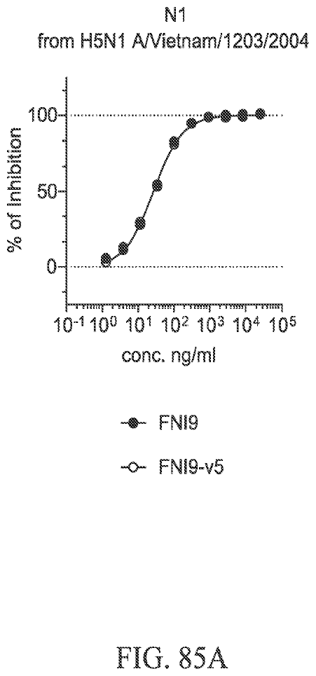

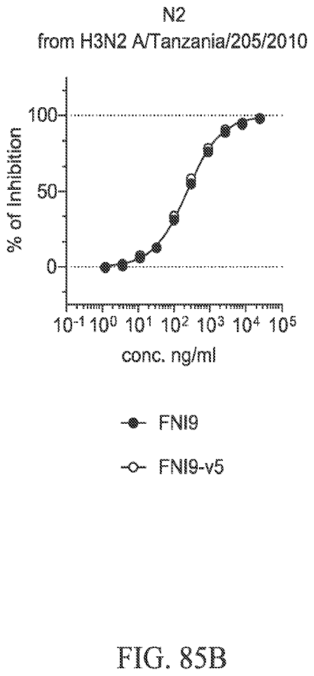

Binding and neutralization by the FNI9 variant antibody FNI9-v5 was evaluated in vitro. Inhibition of sialidase activity by FNI9 and FNI9-v5 against NAs was measured in vitro by MUNANA assay. For the MUNANA assay, FNI9 and FNI9-v5 antibodies were serially diluted and mixed with a fixed amount of purified neuraminidase in 96-well plates and, after a short incubation time, the substrate (MUNANA; 2′-(4-Methylumbelliferyl)-α-D-N-acetylneuraminic acid) was added. The plates were incubated for 2.5 h at 37° C., after which, the reaction was stopped and NA activity was measured by fluorescence. Inhibition of sialidase activity against N1 from H5N1 A/Vietnam/1203/2004 (FIG. 85A) and N2 from H3N2 A/Tanzania/205/2010 (FIG. 85B) was measured.

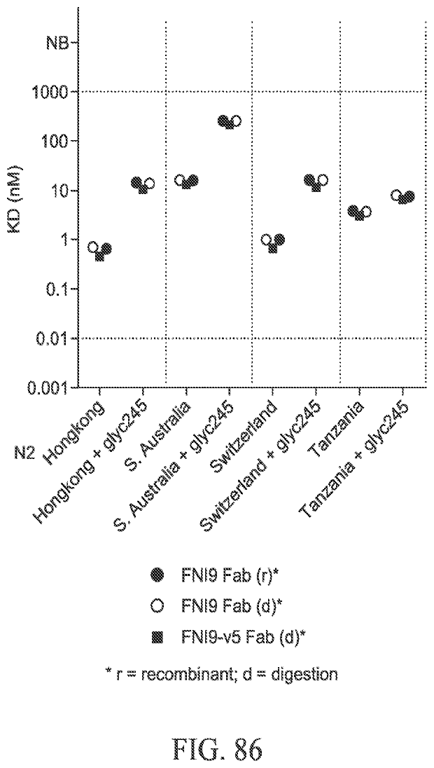

Binding affinity of FNI9 and FNI9-v5 Fab fragments to N2 antigens with or without a glycosylation site at position 245, was measured by surface plasmon resonance (SPR; FIG. 86).

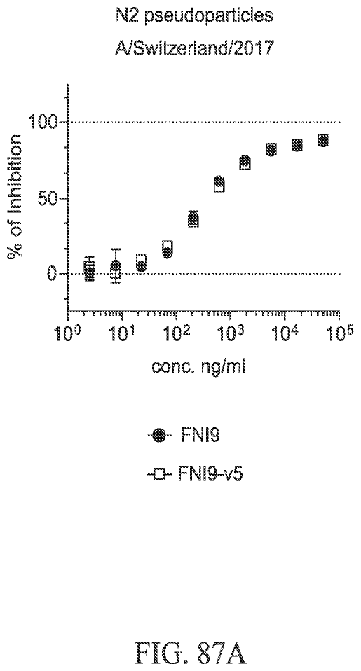

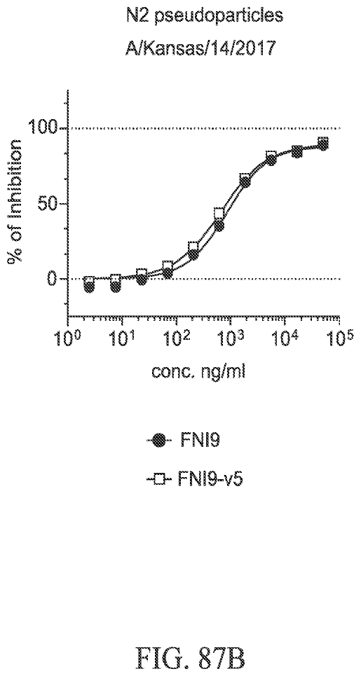

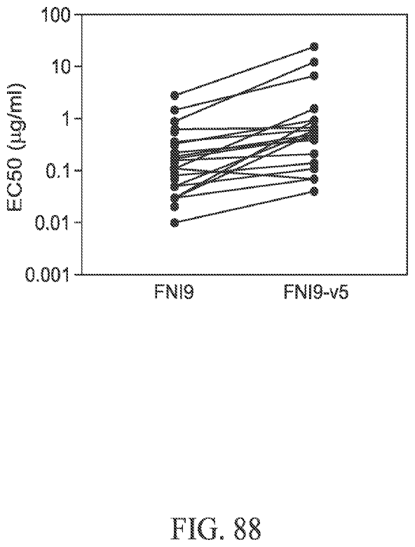

Inhibition of N2 sialidase activity in pseudotype virus particles by FNI9 and FNI9-v5 was measured in vitro by ELLA (enzyme-linked lectin assay). Neuraminidase (NA) activity was quantified using ELLA by measuring the amount of galactose exposed when sialic acid is cleaved by NA sialidase activity. FNI9 and FNI9-v5 antibodies were serially diluted and mixed with a fixed amount of NA-virus-like-particles (pseudoparticles bearing NA only). After a short incubation time, the mix was transferred to fetuin-coated plates. After an overnight incubation at 37° C., peanut-agglutinin (PNA) conjugated with horseradish peroxidase (HRP) was added for detection. The colorimetric read-out was proportional to NA activity. Inhibition of sialidase activity against A/Switzerland/2017 (FIG. 87A) and A/Kansas/14/2017 pseudovirus particles (FIG. 87B) was measured.

### Example 13

### Additional Engineering in FNI9 Antibodies

A reduction of activity was observed for FNI17-v19 against contemporary H3N2 strains carrying a glycan at position 245 of the NA. As compared to FNI17-v19, FNI9-v5 and FNI9-v13 demonstrated similar breadth and potency but higher neutralizing activity against the glycan-bearing NAs. FNI9-v5 was selected over FNI19-v13 for further studies due to higher activity against N9 glycoprotein. However, FNI9-v5 showed lower neutralizing activity than parental FNI9 against some IAV and IBV strains. FNI9 comprises the VH amino acid sequence of SEQ ID NO:2 and the VL amino acid sequence of SEQ ID NO.: 8. Additional FNI9 variant antibodies were generated by introducing one or more amino acid mutations in the VH and/or the VL of parental FNI9. See FIGS. 70-72. The variants summarized in FIG. 72 are:

| Variant | VH amino acid | VL amino acid |
|---|---|---|
| Antibody | SEQ ID NO.: | SEQ ID NO.: |
| FNI9-v13.8 | 65 | 68 |
| FNI9-v4.1 | 46 | 8 |
| FNI9-v5.1 | 48 | 8 |
| FNI9-v6.1 | 50 | 8 |
| FNI9-v7.1 | 52 | 8 |
| FNI9-v8.1 | 54 | 8 |
| FNI9-v9.1 | 56 | 8 |
| FNI9-v10.1 | 58 | 8 |
| FNI9-v11.1 | 60 | 8 |
| FNI9-v12.1 | 63 | 8 |
| FNI9-v4.7 | 46 | 66 |
| FNI9-v5.7 | 48 | 66 |
| FNI9-v6.7 | 50 | 66 |
| FNI9-v7.7 | 52 | 66 |
| FNI9-v8.7 | 54 | 66 |
| FNI9-v9.7 | 56 | 66 |
| FNI9-v10.7 | 58 | 66 |
| FNI9-v11.7 | 60 | 66 |
| FNI9-v12.7 | 63 | 66 |

Also generated was a variant antibody comprising the VH amino acid sequence set forth in SEQ ID NO:2 and the VL amino acid sequence set forth in SEQ ID NO:37.

Also generated was a variant antibody comprising the VH amino acid sequence set forth in SEQ ID NO:45 and the VL amino acid sequence set forth in SEQ ID NO.: 8.

An example of a FNI9-v8.1 full-length heavy chain amino acid sequence is provided in SEQ ID NO.: 107. An example of a polynucleotide encoding this full-length heavy chain is provided in SEQ ID NO. 109. An example of a FNI9-v8.1 full-length light chain amino acid sequence is provided in SEQ ID NO. 108. An example of a polynucleotide encoding this full-length light chain is provided in SEQ ID NO.: 110.

### Example 14

### Design of Novel FC Variants

Human IgG1 Fc regions were engineered for improved function, such as to potentially promote prophylactic, therapeutic, or vaccinal effects by activating certain FcγRs (e.g. FcγRIIA, FcγRIIIA, FcγRIIB). Enhancing activation of FcγRIIA in early infection may promote antibody-dependent cellular phagocytosis (ADCP) and viral neutralization. Enhancing activation of FcγRIIA and/or FcγRIIIA in late or established infection may promote ADCP and/or antibody-dependent cellular cytotoxicity (ADCC), facilitate clearance of virally infected cells, and block viral spread. Enhancing activation of FcγRIIA and/or FcγRIIIA at any time during infection may provide a vaccinal effect by promoting antigen presentation and adaptive immunity.

Fc variants were assessed and new variants were developed using an iterative discovery workflow. An initial set of approximately 2500 Fc point mutations was generated, and functional data was collected and analyzed. Functional data included binding interactions (e.g. to FcγRI, FcγRIIA (R131), FcγRIIB, FcγRIIC, FcγRIIIA (V158), FcRn, and C1q), signaling via FcγRs, thermostability, expressibility, polyreactivity, and half-life extendibility. A machine learning and multi-factor prediction-based algorithm was developed to assist in designing further variants. Fc variants were expressed as anti-influenza A IgG1 antibodies (with FY1 Fabs; Kallewaard et al. Cell 166(3):596-608 (2016)) in CHO cells, titered using high-performance liquid chromatography (HPLC), and purified using protein A columns. A first plate (2×96, with or without 2-deoxy-2-fluoro-L-fucose (2FF), which inhibits fucosylation) contained wells for measuring effects of known mutations (as reference) and wells for measuring effects of novel mutations (single or combination).

Fc variants were analyzed using various assays to evaluate biophysical, biochemical, and biological properties. These included aggregation (e.g. by size-exclusion chromatography), thermostability, glycosylation, structure, signaling, and binding (e.g. using surface plasmon resonance or meso scale discovery-based assays). Effector functions were also tested, including antibody-dependent cellular cytotoxicity (ADCC) and antibody-dependent cellular phagocytosis (ADCP). Binding characteristics of single Fc mutations were evaluated, combinations of up to three mutations were identified that had the highest effect on increasing the IIA/IIB ratio, and additional variations included. The resulting further variants were analyzed. Characteristics of interest included increased affinity for FcγRIIa with reduced affinity for FcγRIIb, or vice versa. Using unbiased cluster analysis and radar plotting with manual analysis, nine clusters of Fc variants having strongly increased, increased, similar or the same, decreased, or strongly decreased affinity for various FcγRs and FcRn were identified.

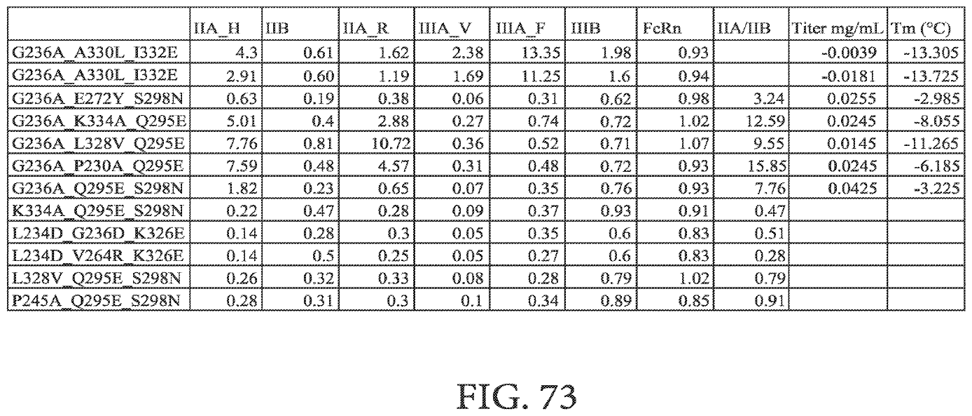

Binding affinities (measure by MSD), Tm, and production titers for certain Fc variants are shown in FIG. 73. Of ten variants that were initially predicted to increase the ratio of FcγRIIA/FcγRIIB binding, five of these (with IIA/IIB values shown in bold in FIG. 73) increased the ratio, as measured by MSD.

### Example 15

### Further Testing of FC Variants

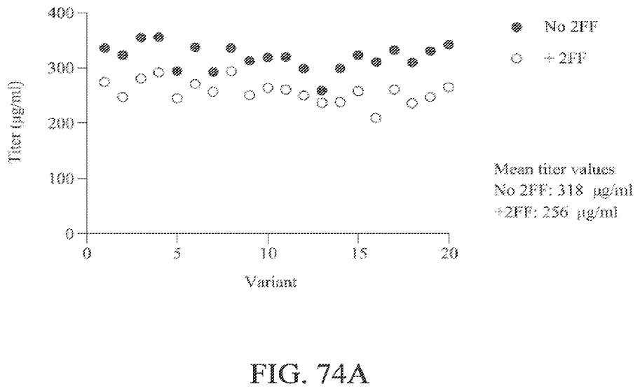

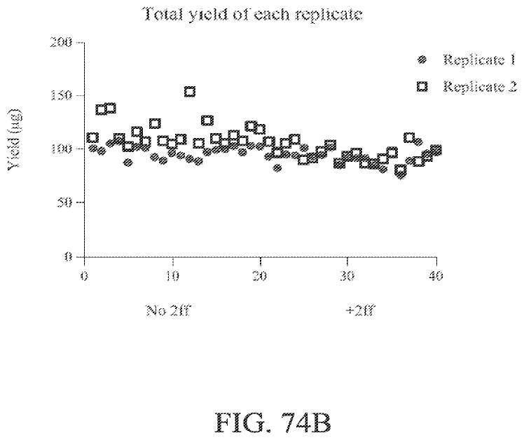

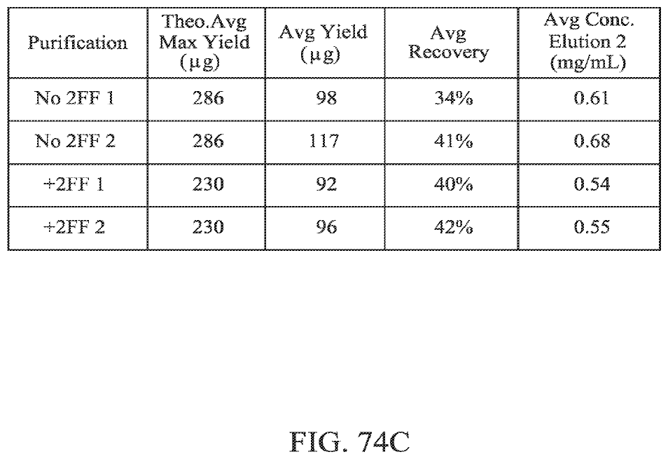

Based on results obtained from the first plate of variants described in Example 14, a second plate (2×20, with and without 2FF) of variants was generated. The twenty Fc variant antibodies were expressed and purified to evaluate titer and yield. Variants were expressed without or with 2FF to determine the effect of fucosylation on titer and yield (FIGS. 74A-74C). FIG. 4A shows antibody titers as determined using a Protein A column. Mean titer was higher for variants expressed in cells without 2FF. FIG. 74B shows yields resulting from two replicate purifications (input volume of 900 μL), with two elutions per purification. FIG. 74C summarizes the theoretical maximum yield, average yield, average recovery and protein concentration of the second elution (measured in μg/ml). Fc variants were purified using two elutions and combined prior to determining yield. Average yield was higher in purified Fc variants expressed without 2FF.

Purified antibodies (+2FF and no 2FF) were analyzed for potential to dimerize using size-exclusion chromatography and Tm were evaluated. Low molecular weight species were observed for C220P and R292P_Y300L variants (+2FF samples only). Tm values for certain Fc variants were within 4.2° C. of WT; by comparison, Tm of the GAALIE Fc variant was approximately 14° C. lower relative to WT (tested on two plates). CH2 unfolding could not be resolved for variants including R292P except for the combination mutant G236A_R292P_1377N.

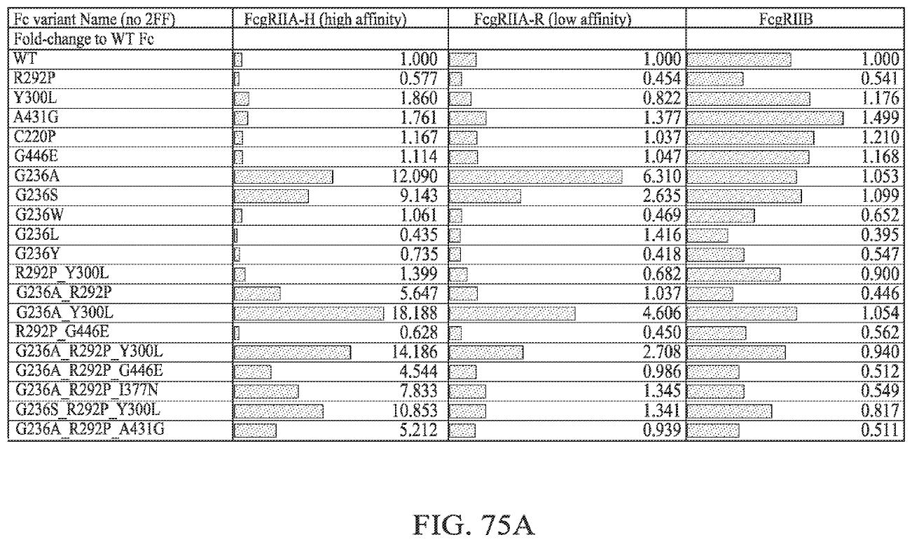

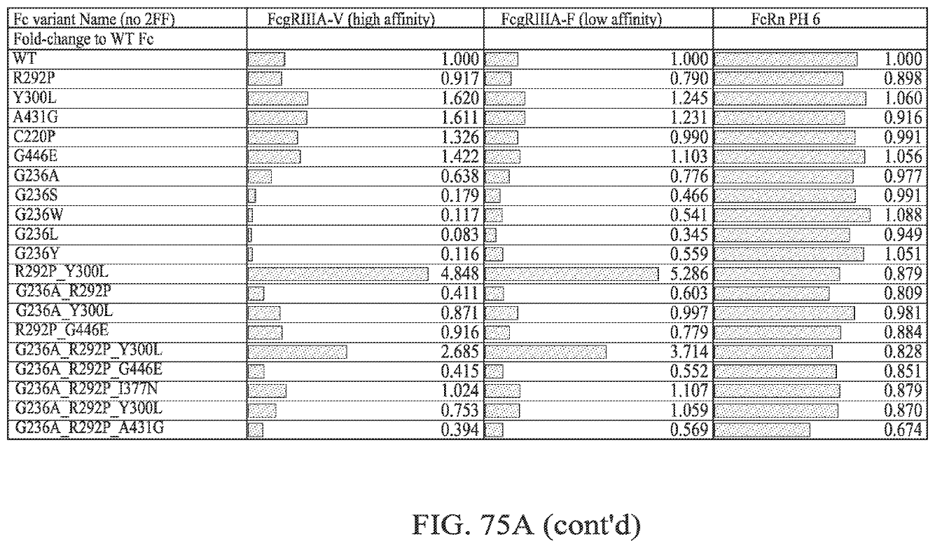

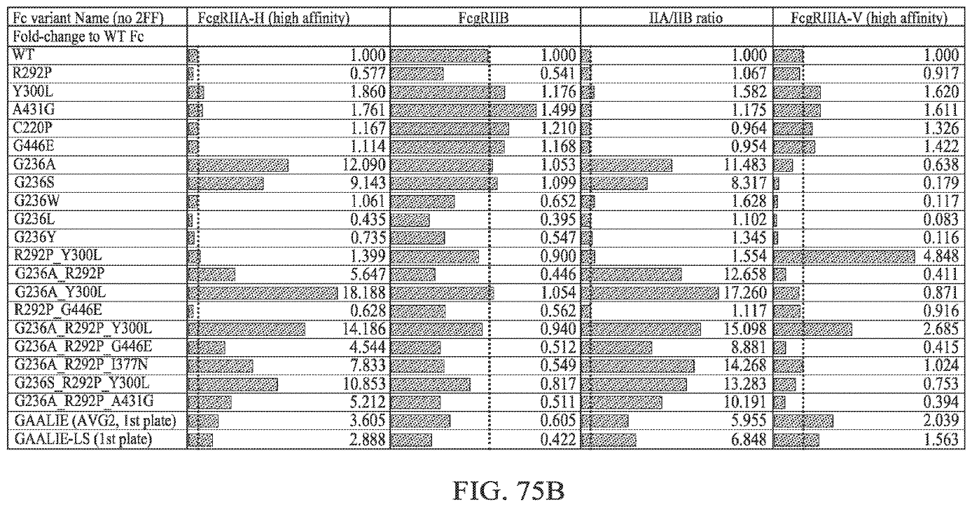

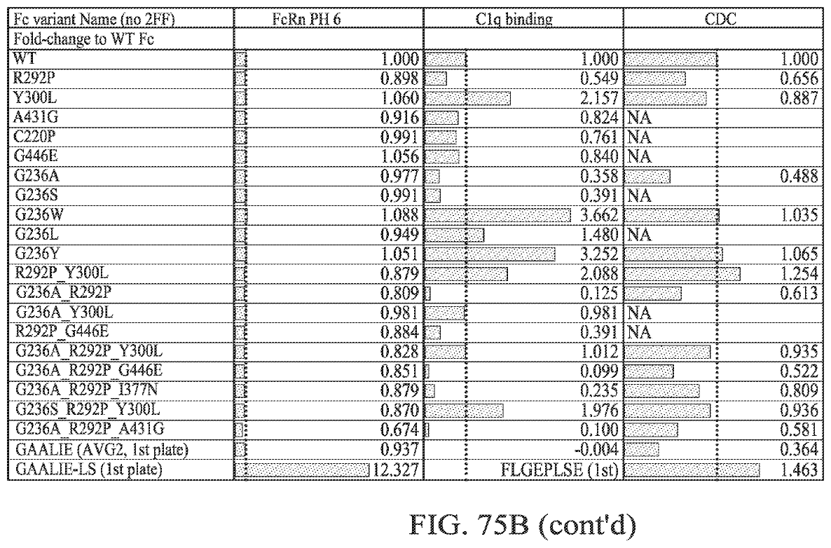

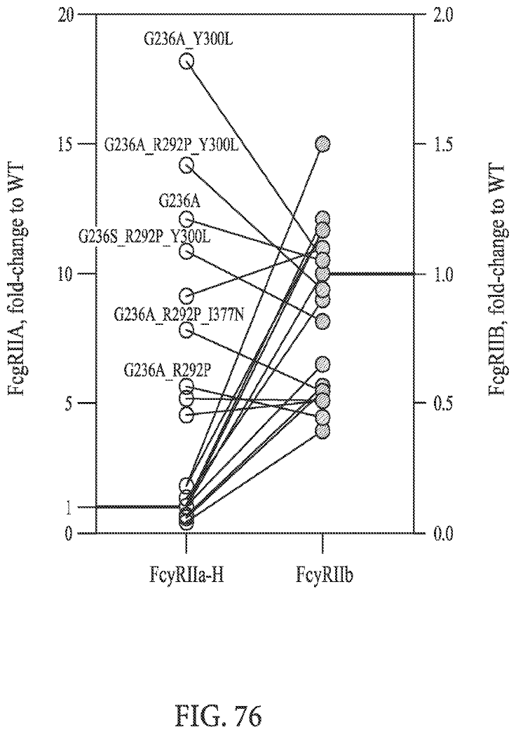

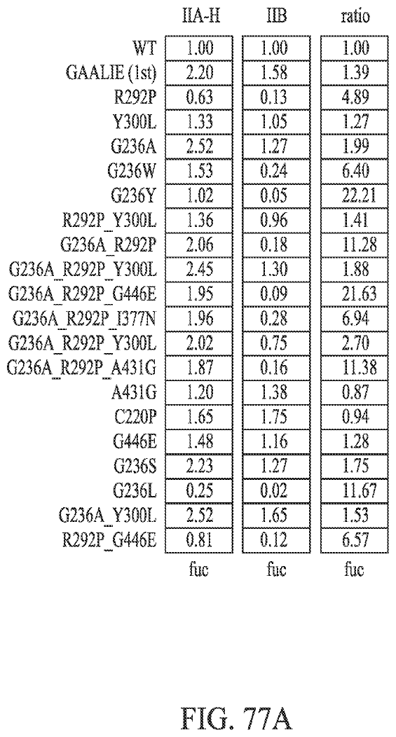

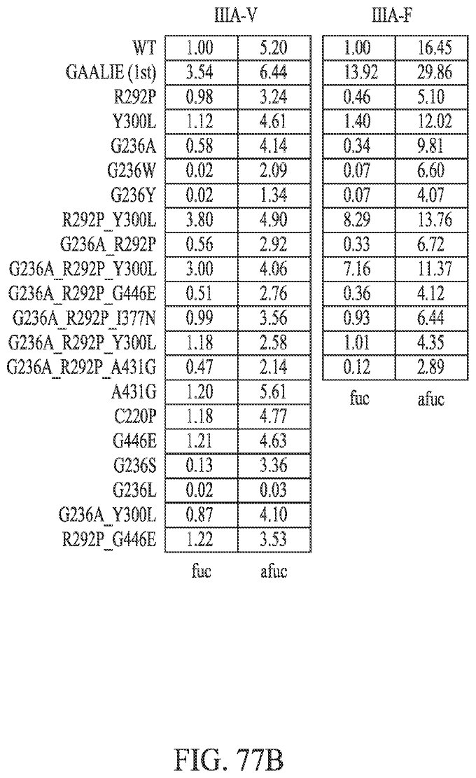

Binding of (No 2FF) Fc variants to FcγRIIA-H (high affinity), FcγRIIA-R (low affinity), FcγRIIB, FcγRIIIA-V (high affinity), FcγRIIIA-F (low affinity), and FcRn (at pH 6) was tested and expressed as fold-change relative to wild-type Fc (FIG. 75A). The ratio of FcγRIIA-H/FcγRIIB binding, as well C1q binding and complement-dependent cytotoxicity (CDC) data, are also shown in FIG. 75B. Variants shown in FIG. 76 were not treated with 2FF. Antibody signaling through different FcγRs was measured using a reporter assay (Promega™ luciferase reporter cells; average of 3 experiments). Fucosylated Fc variants were tested for signalling through all four FcγR receptors shown (FIG. 77A), while afucosylated variants were tested for signalling through FcγRIIIA-V and FcγRIIIA-F (FIG. 77B). A number of variant Fcs were selected for further characterization.

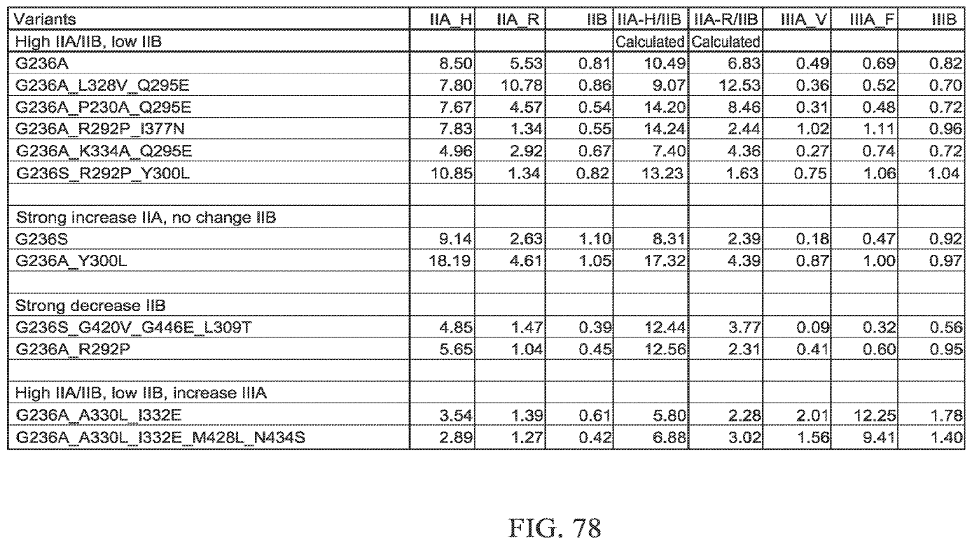

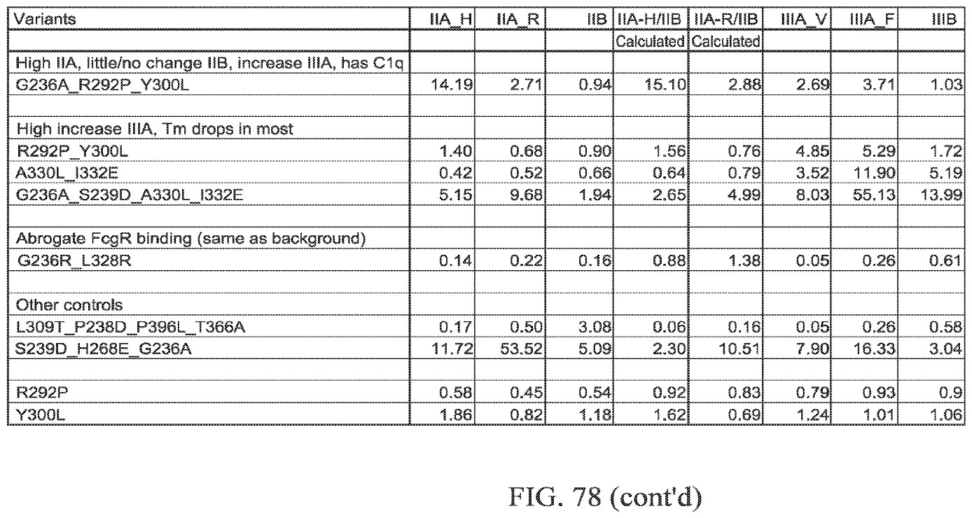

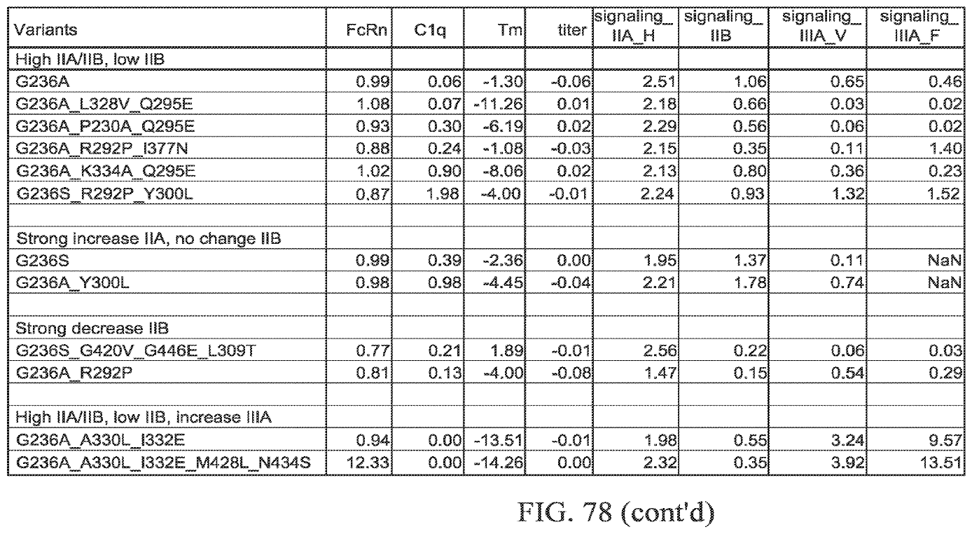

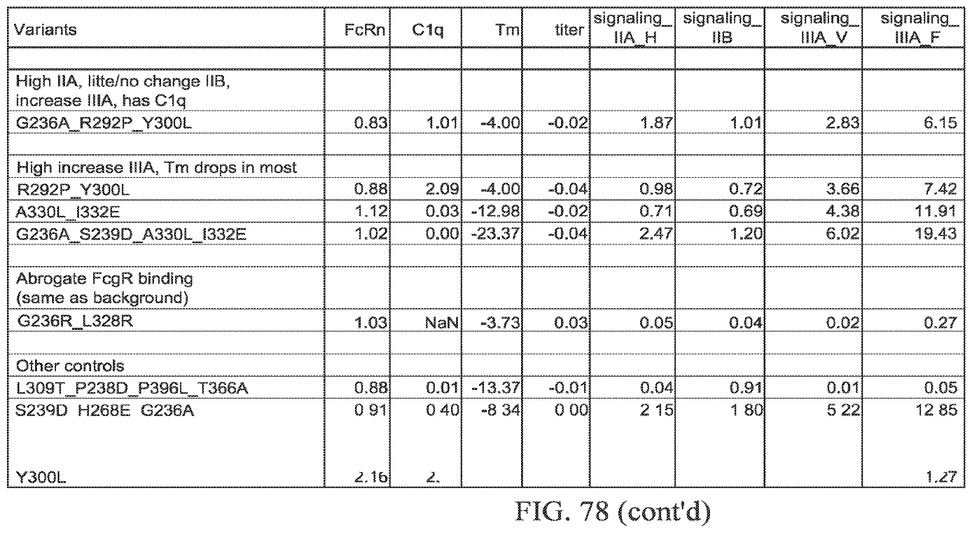

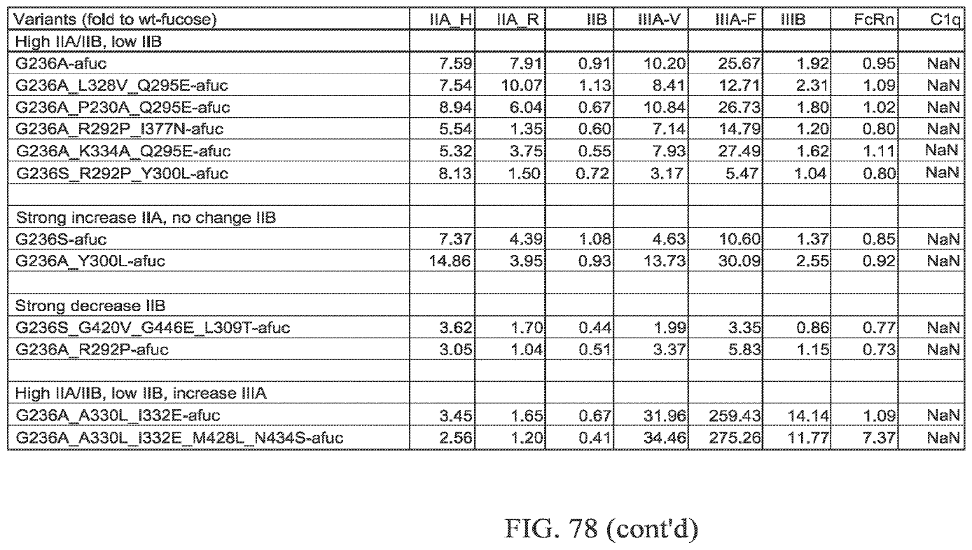

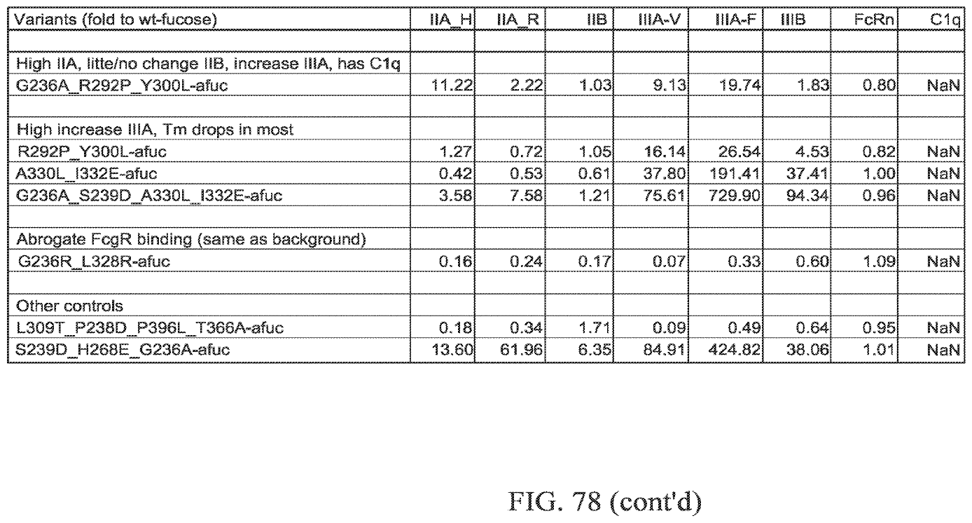

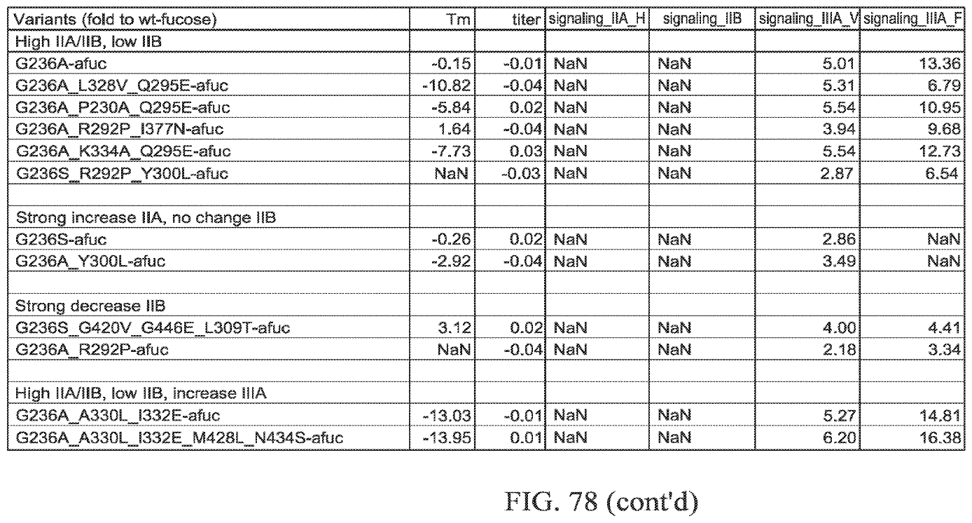

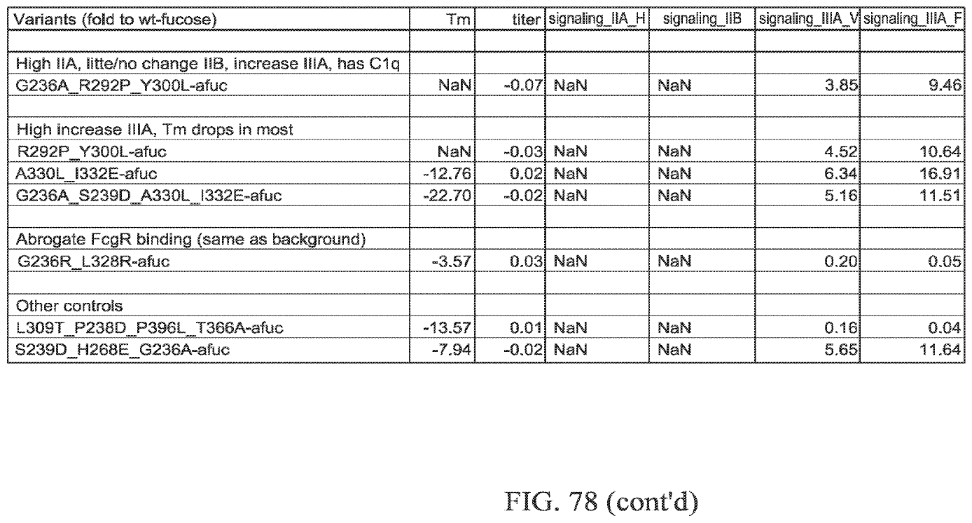

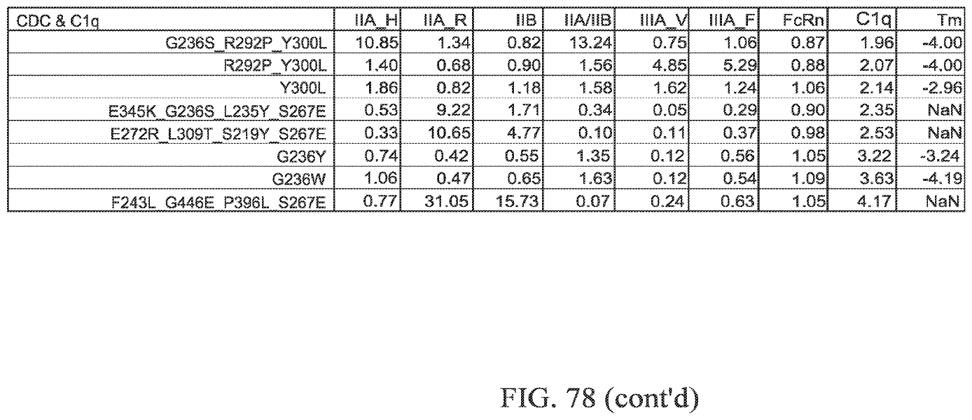

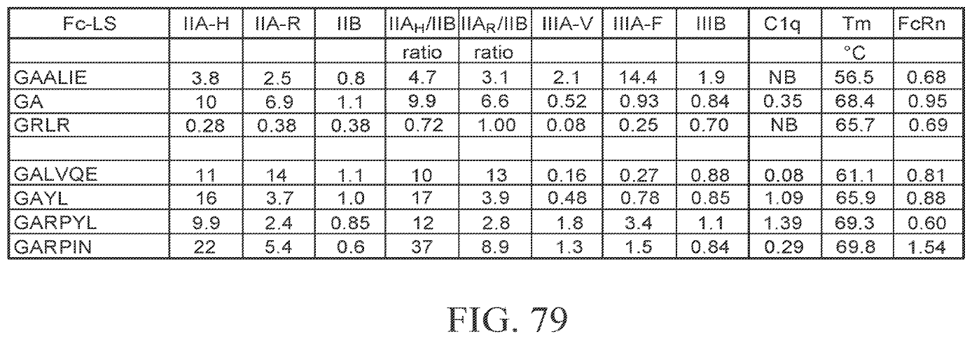

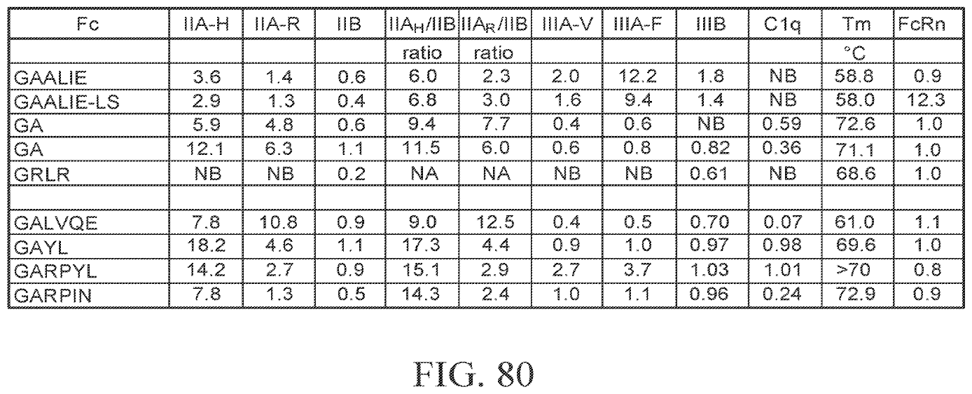

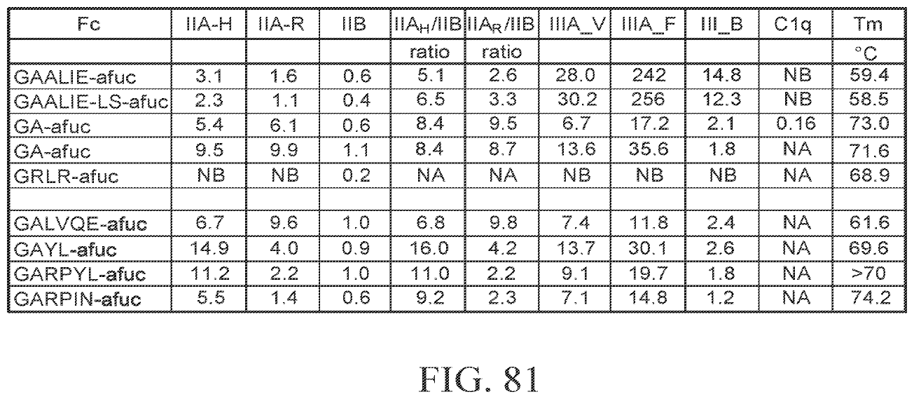

A summary of characteristics of these variants (both fucosylated and afucosylated), as well as of comparator variant Fcs comprising known mutations (e.g., G236A_S239D_A330L_I332E (“GASDALIE”); G236A_A330L_I332E (“GAALIE”) is shown in FIG. 78.

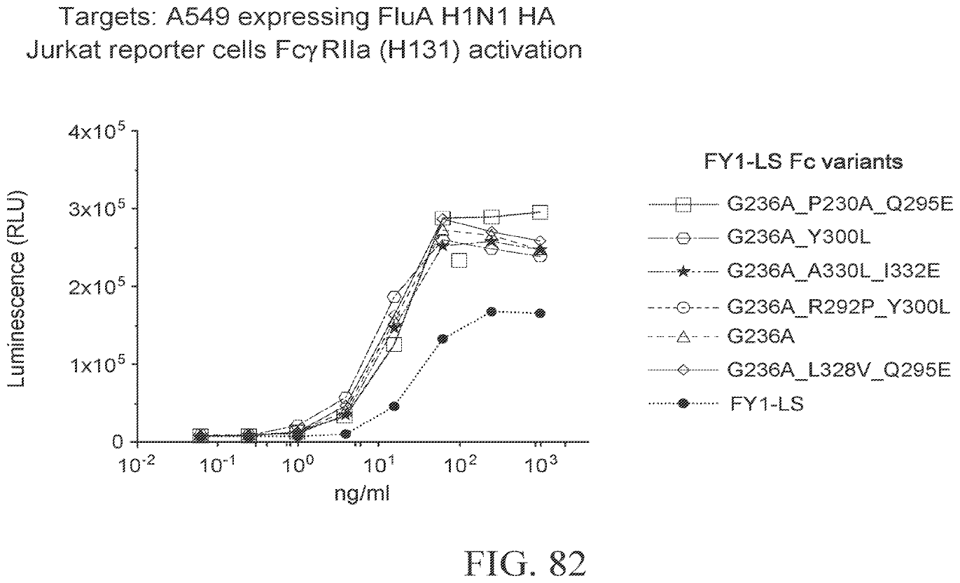

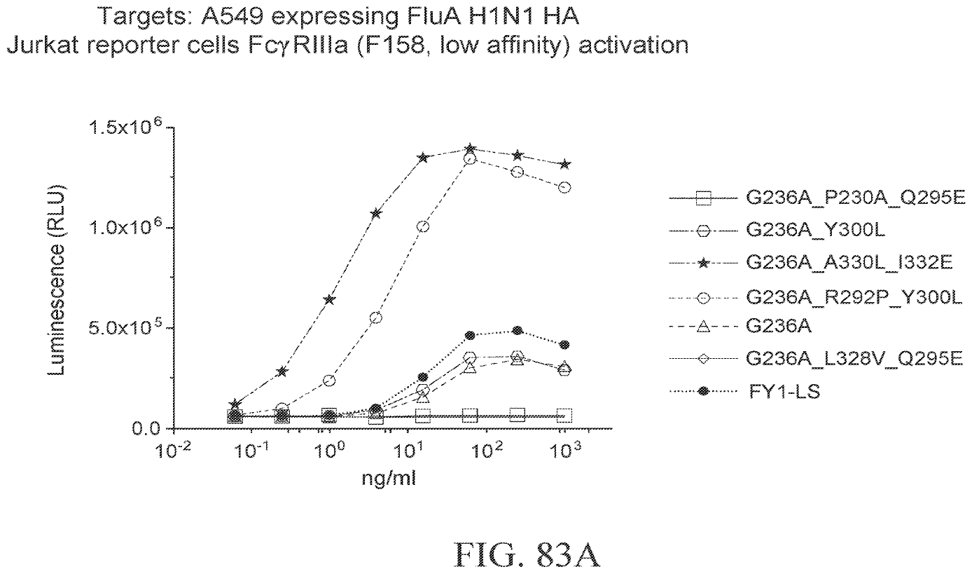

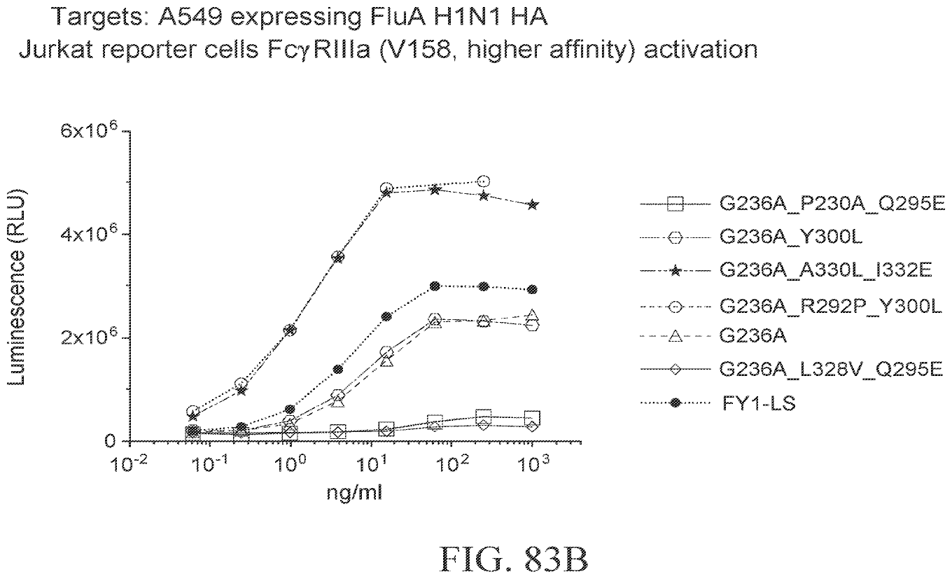

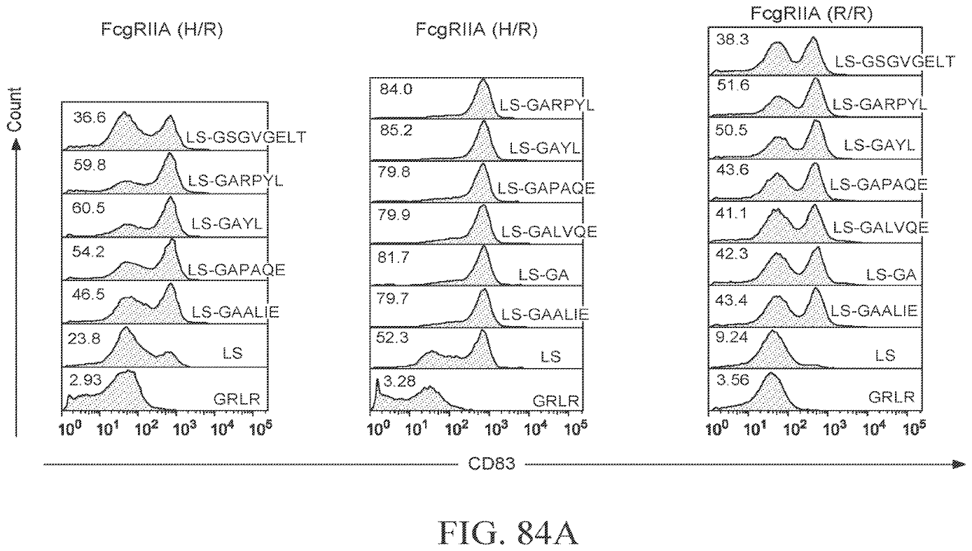

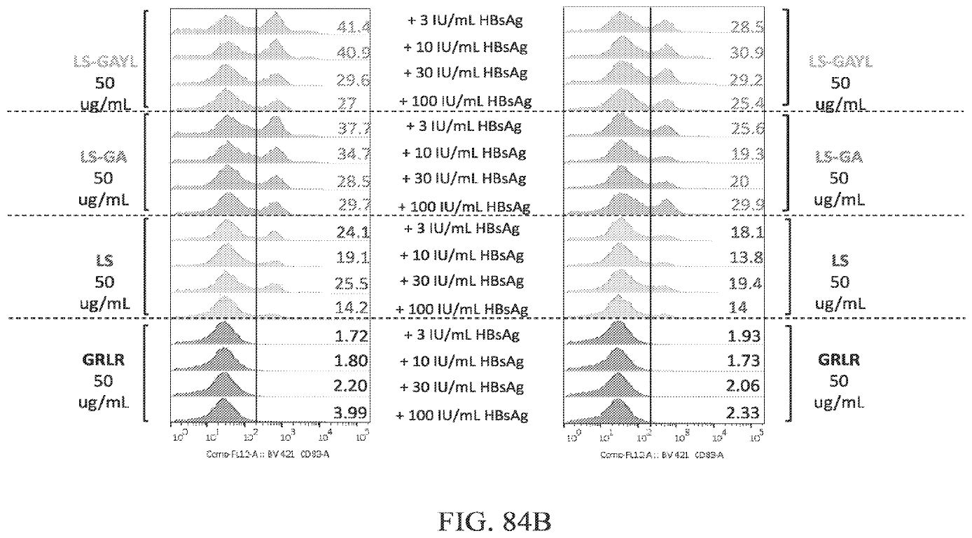

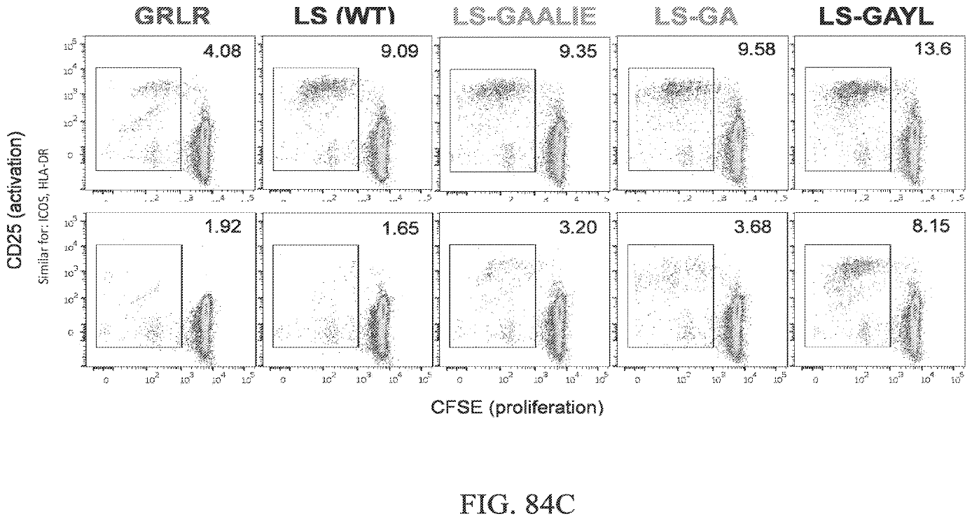

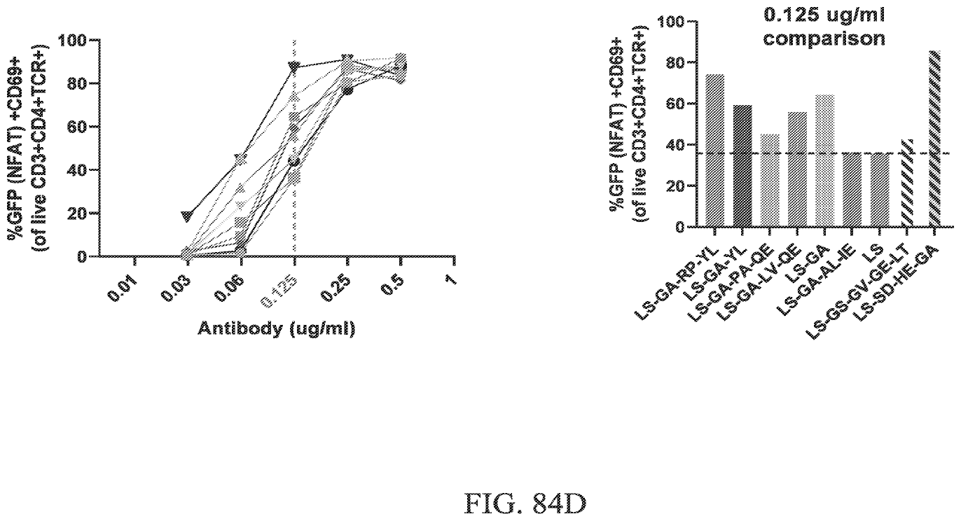

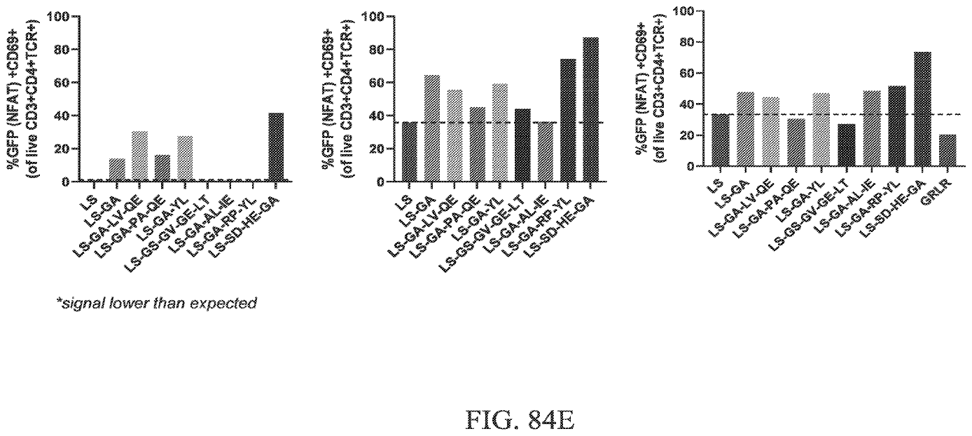

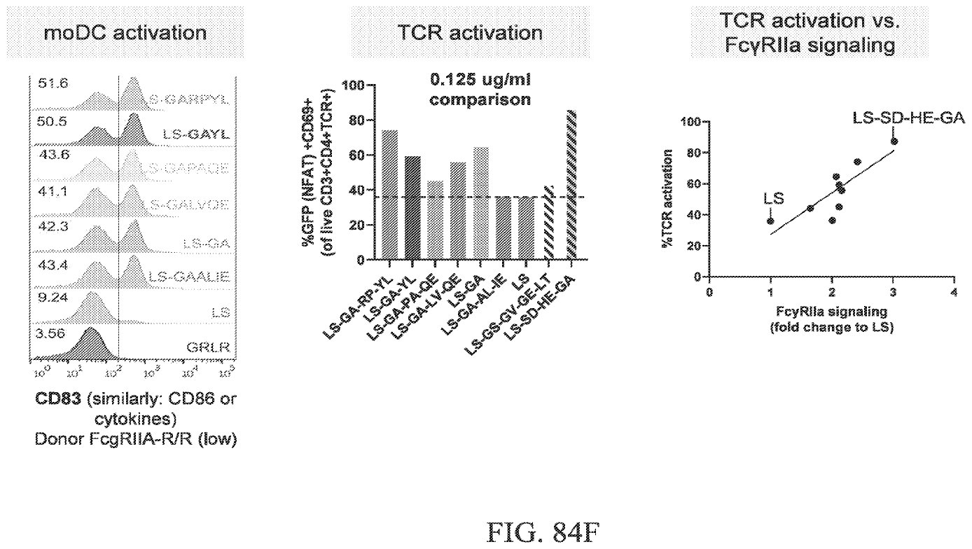

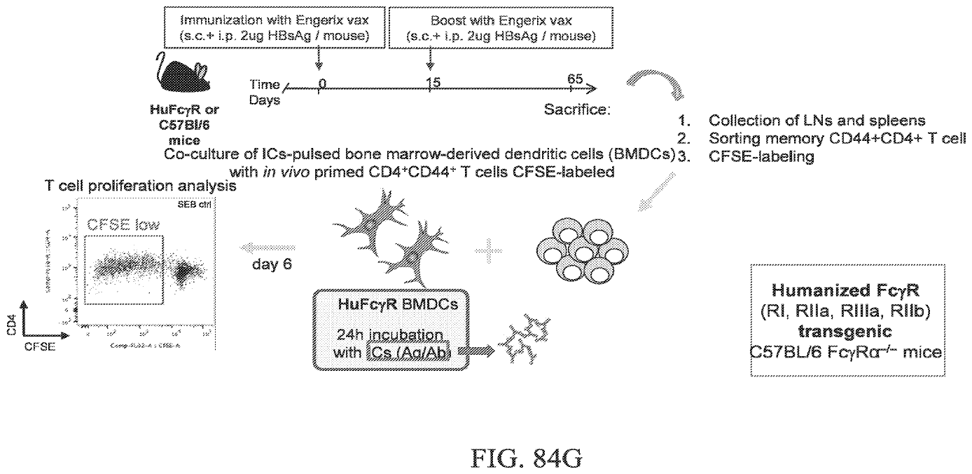

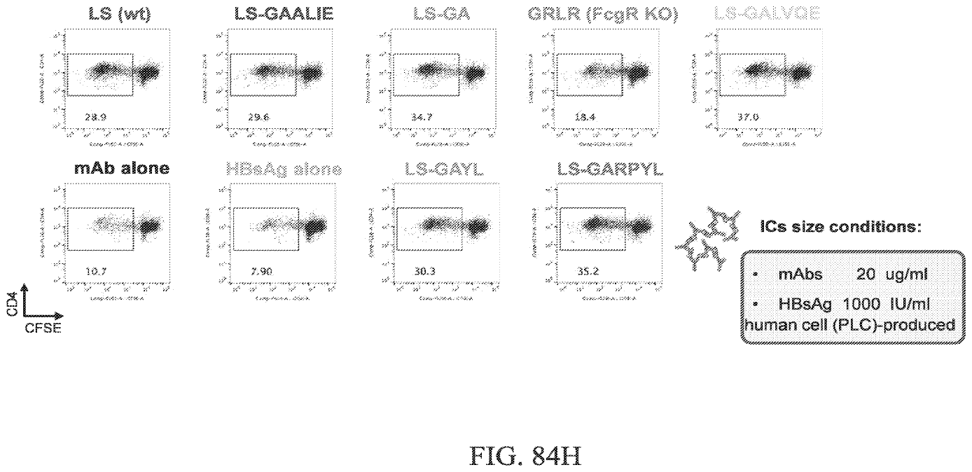

Further studies were performed as shown and described in FIGS. 82-84I. FIGS. 82-83B show FcγR activation and binding by anti-influenza (HA) antibody “FY1” Fc variants. FIGS. 84A-84I relate to anti-HBsAg (HBC34-v40) Fc variant antibodies and show: activation of and cytokine production by human monocyte-derived dendritic cells (moDCs) using antibodies and HBsAg; activation of human HBsAg-specific CD4+ memory T cells; activation of HBsAg-specific TCR-transgenic Jurkat reporter CD4+ T cells; re-stimulation of CD4+ memory T cells from HBV-vaccinated huFcγR mice by antibody:HBsAg immune complexes (ICs); and binding kinetics (including fold-change vs. control Fc) of HBC34-v40 Fc variants for human FcγRs.

### Example 16

### Testing of Additional FNI9 Variant Antibodies

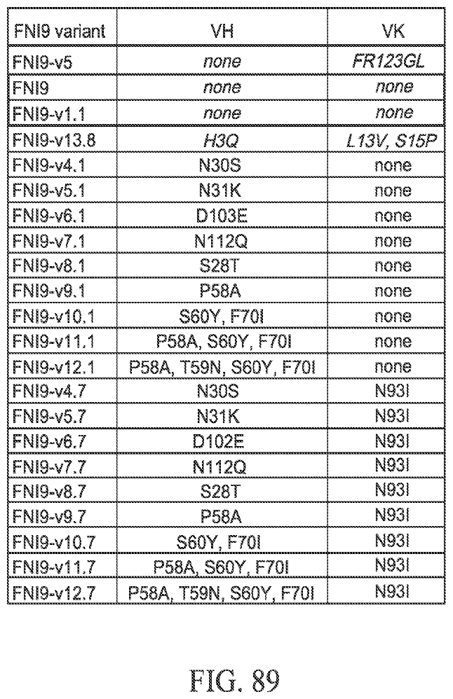

FNI9 variant antibodies as described in Example 13 were further characterized. The “FNI9” and “FNI9-v1.1” antibodies shown herein (FNI9-v1.1 is referenced in, for example, FIGS. 89-93D, 95A, 95B, 96A, 97A, 97B, and other figures), have the same VH and VK amino acid sequences. FNI9-v1.1 was produced concurrently with the FNI9 variants to control for any variance in production conditions.

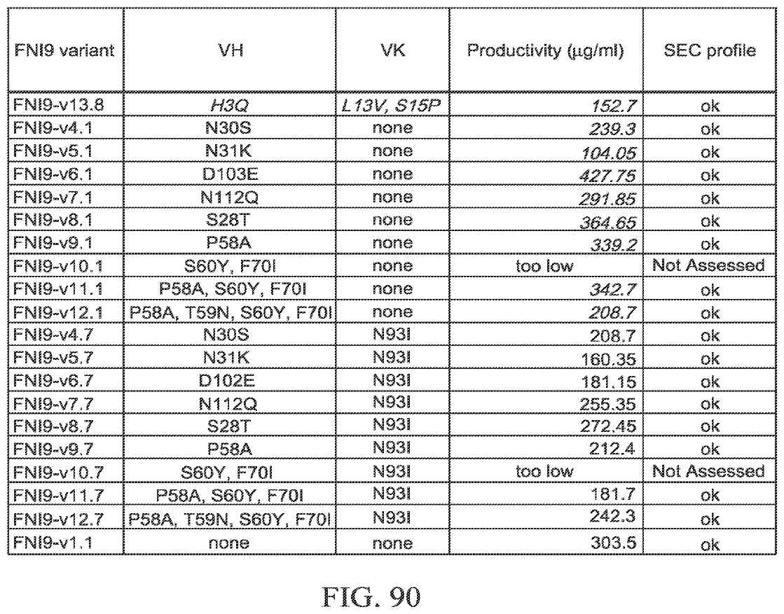

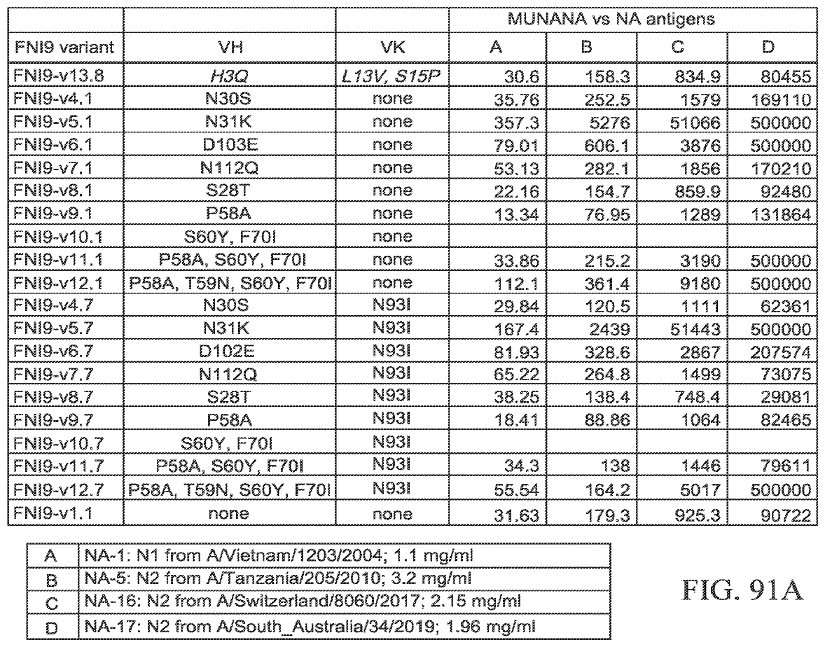

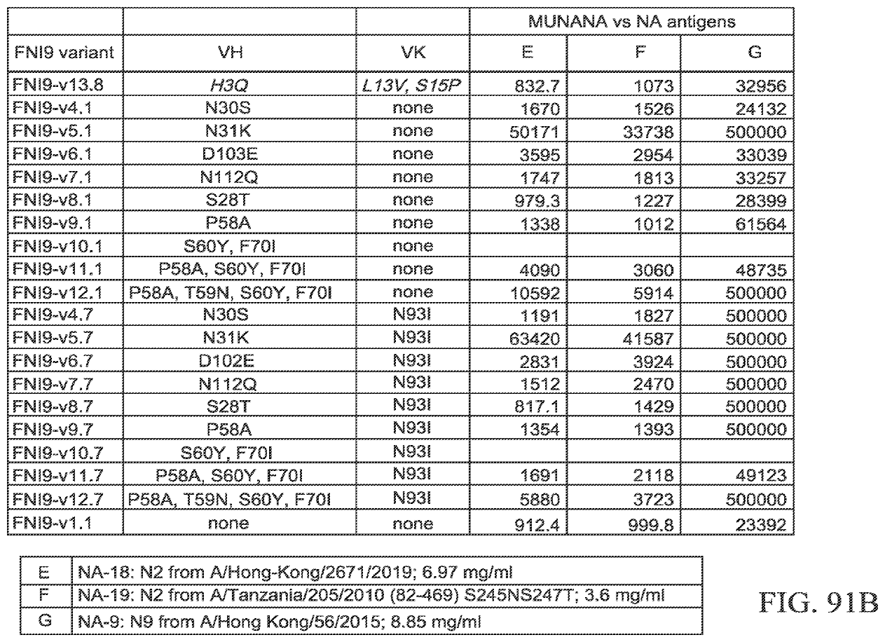

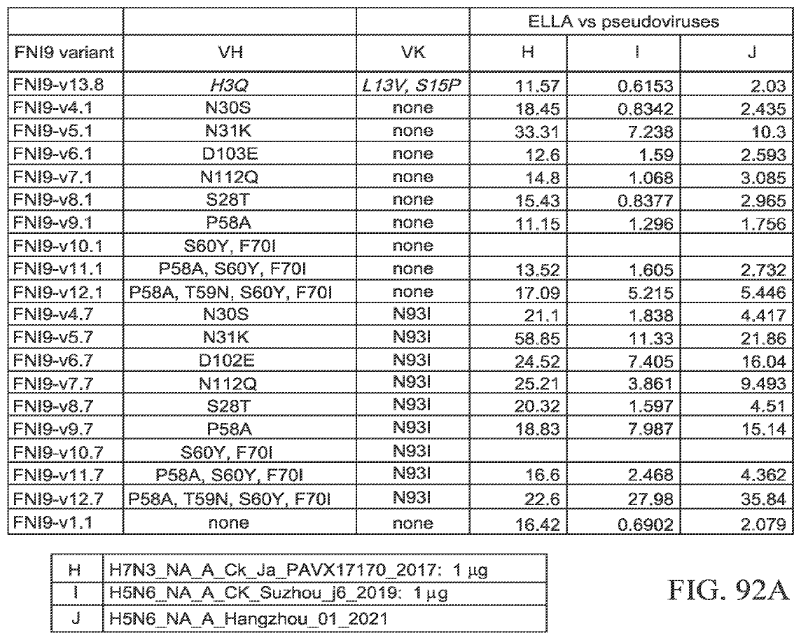

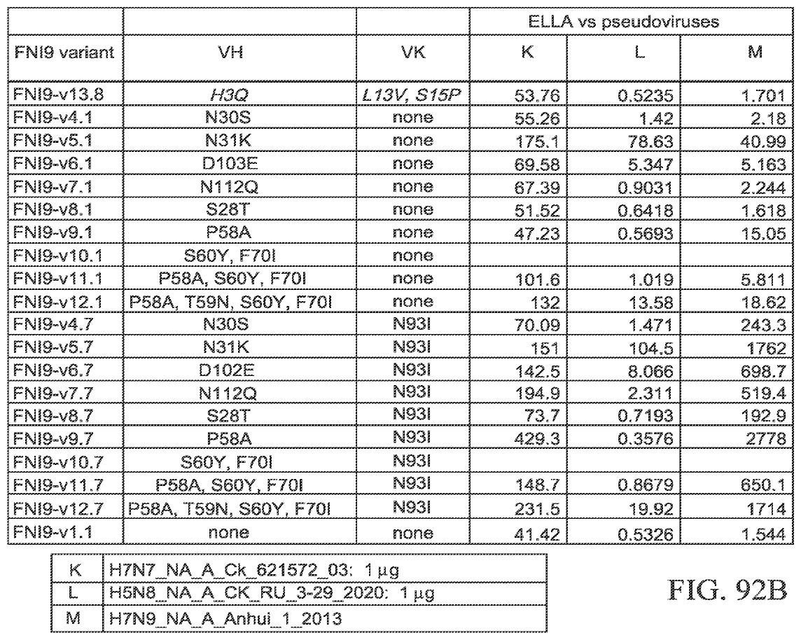

Production titer and size exclusion chromatography (SEC) profiles for FNI9 variant antibodies are shown in FIG. 90. In vitro inhibition of sialidase activity by FNI9 variants against N1, N2, and N9 NAs was measured by MUNANA assay (FIGS. 91A-91B). In vitro inhibition of sialidase activity by FNI9 variants against pseudovirus-derived NAs was measured by ELLA (FIGS. 92A-92B).

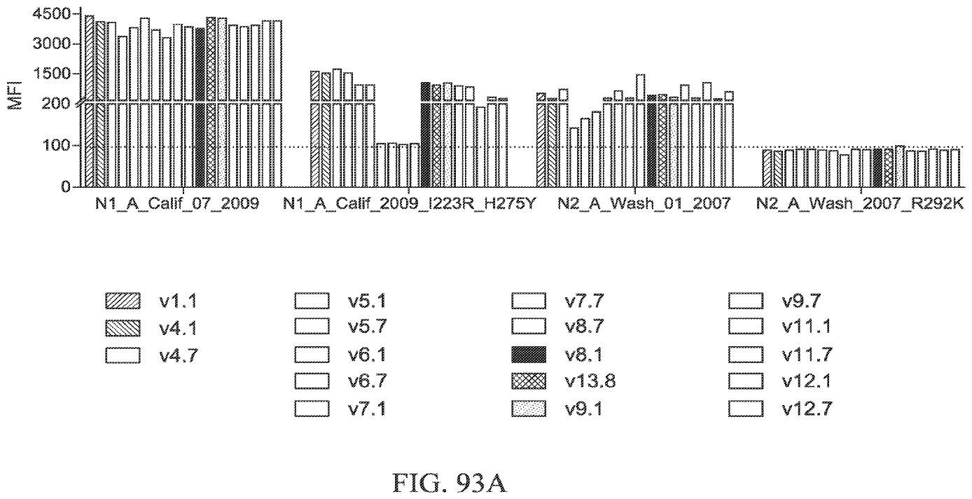

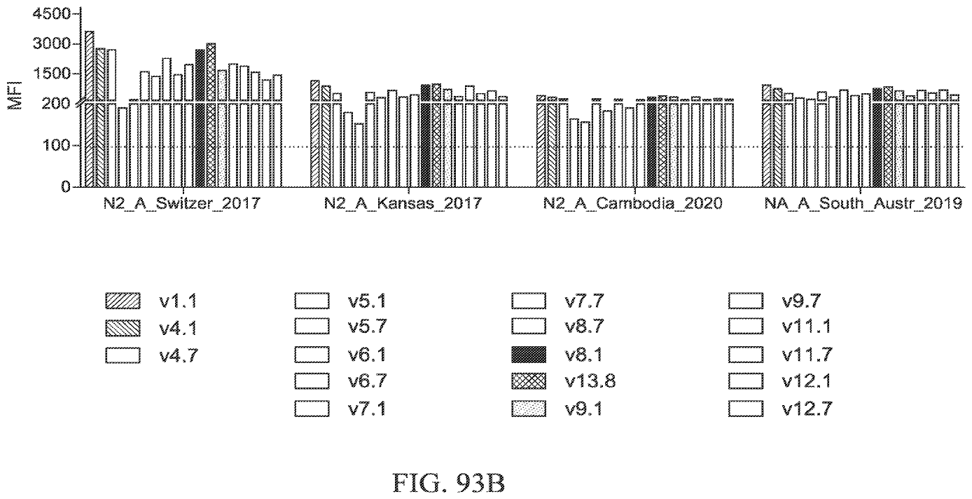

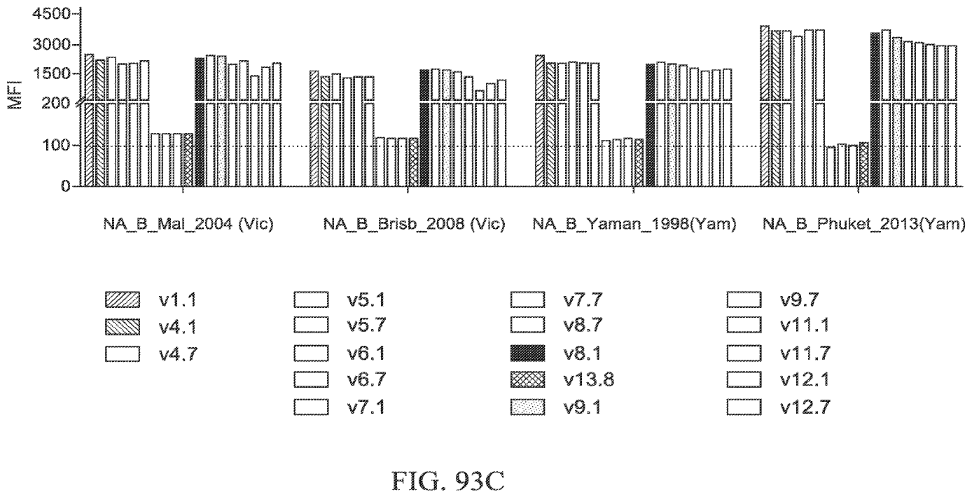

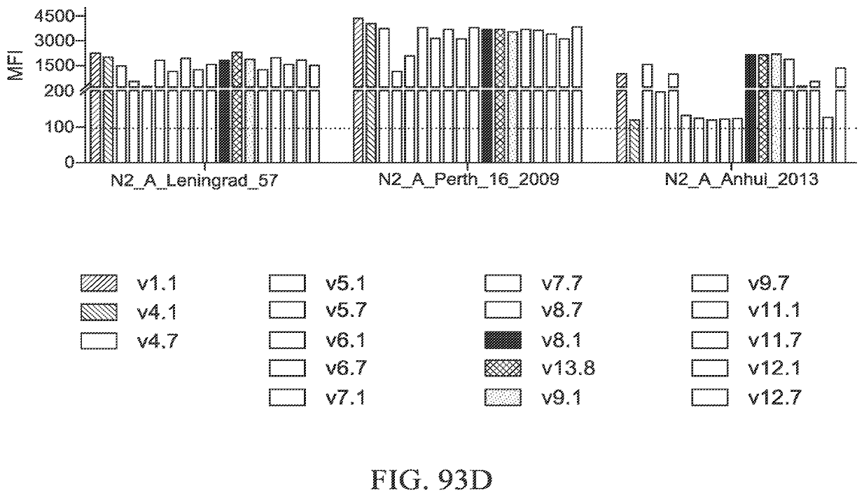

Binding of FNI9 variant antibodies to NAs transiently expressed in mammalian cells was measured by flow cytometry (FIGS. 93A-93D). FIG. 93A shows binding to N1 from A/California/07/2009 and A/California/07/2009 I223R/H275Y, and N2 in A/Washington/01/2007 and A/Washington/01/2007 R292K. FIG. 93B shows binding to N2 from A/Switzerland/8060/2017, A/Kansas/14/2017, A/Cambodia/2020, and A/South Australia/34/2019. FIG. 93C shows binding to IBV NAs from B/Malaysia/2506/2004 (Victoria), B/Brisbane/2008 (Victoria), B/Yamanashi/166/1998 (Yamagata), and B/Phuket/3073/2013 (Yamagata). FIG. 93D shows binding to N2 from A/Leningrad/134/17/57 and A/Perth/16/2009, and N9 from A/Anhui/1/2013.

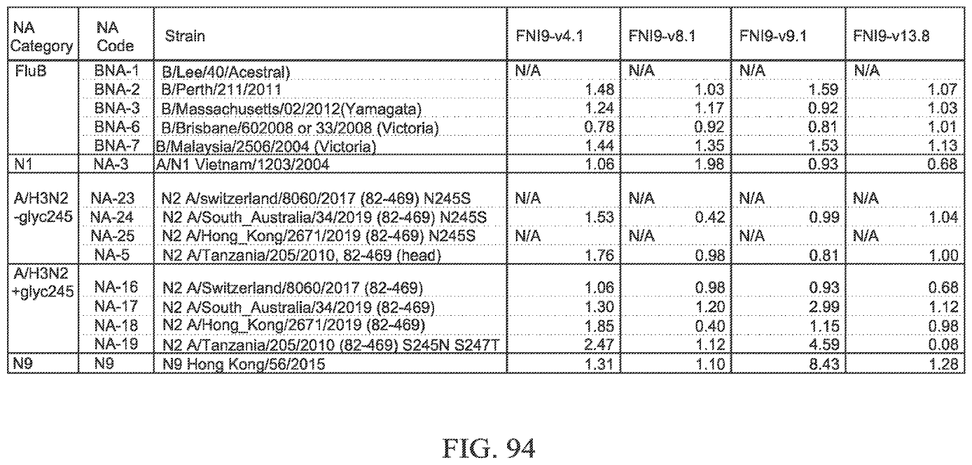

Binding affinity of certain FNI9 variant antibodies for IAV and IBV NA antigens was measured by SPR (FIG. 94) using a Biacore instrument. NAs were immobilized on anti-Avi chips and serial dilutions of each FNI9 variant Fab were added onto the immobilized ligands. The association and dissociation kinetics were measured in real time and a 1:1 binding model was used to extrapolate kinetics parameters and calculate KD values. Affinity was assessed as fold-change compared to the parental antibody, “FNI9-v1.1”. “FNI9-v4.1”, “FNI9-v8.1”, “FNI9-v9.1”, and “FNI9-v13.8” (expressed as IgG1) were tested for binding to IBV NA, IAV N1, IAV N2 (with and without glycosylation at position 245), and IAV N9.

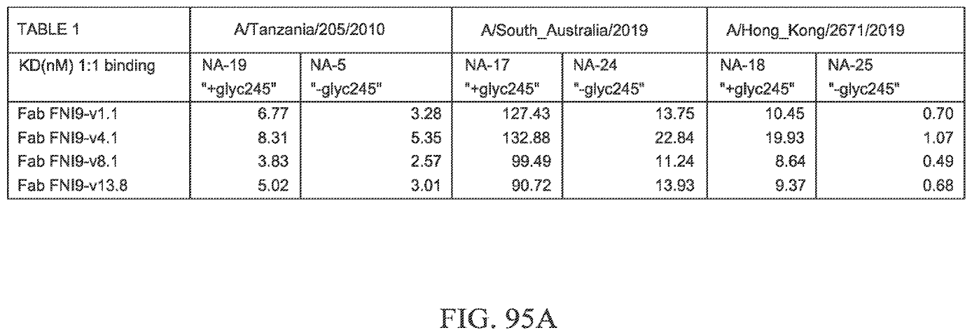

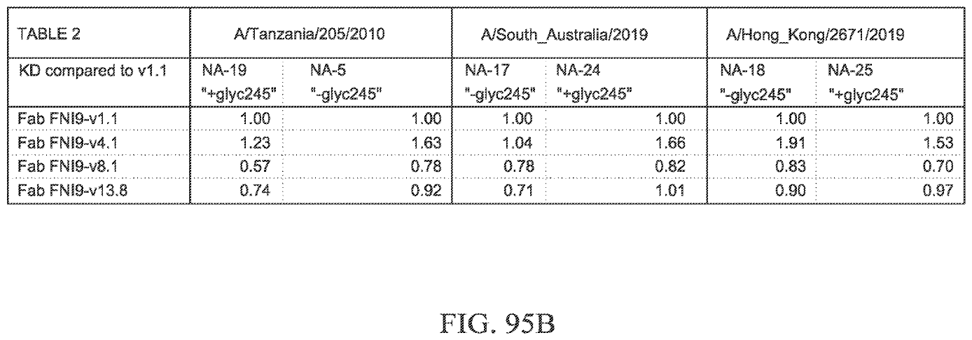

Binding affinity of Fab fragments from FNI9-v1.1, FNI9-v4.1, FNI9-v8.1, and FNI9-v13.8 for N2 antigens was measured by SPR (FIGS. 95A-95B). Binding to A/Tanzania/205/2010, A/South Australia/34/2019, and A/HongKong/2671/2019 with (labelled “+gly245”) or without (labelled “-gly245”) glycosylation at position 245 was tested. FIG. 95A shows binding affinity reported as the equilibrium constant, KD, in nM, while FIG. 95B shows binding affinity reported as fold-change compared to Fab from the parental antibody, FNI9-v1.1.

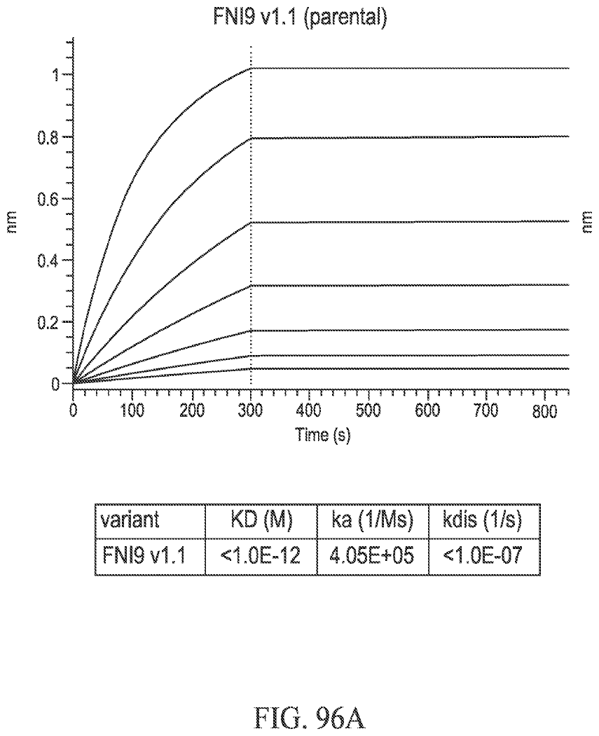

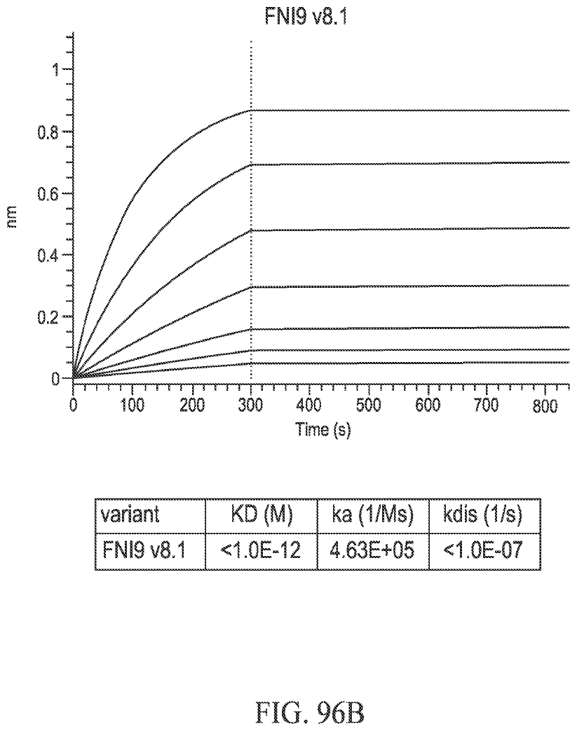

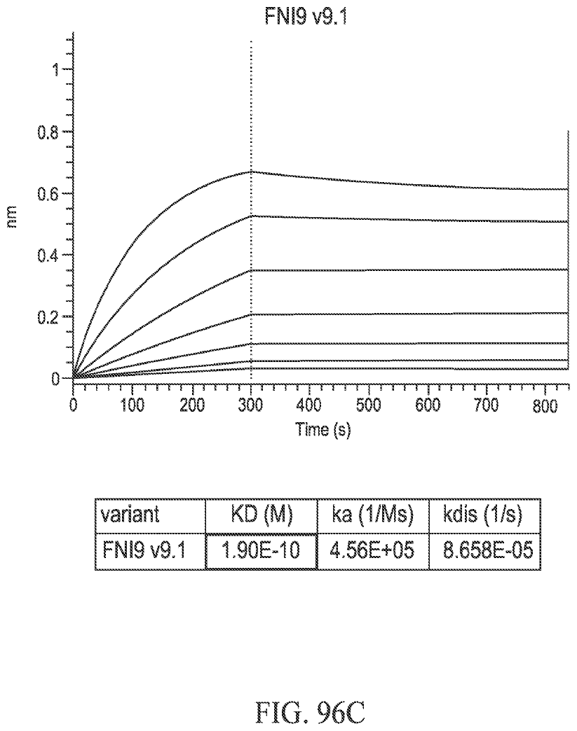

Binding kinetics of FNI9-v1.1 (FIG. 96A), FNI9-v8.1 (FIG. 96B), and FNI9-v9.1 (FIG. 96C) to N9 NAs were measured by Bio-Layer Interferometry (BLI; FIGS. 96A-96C).

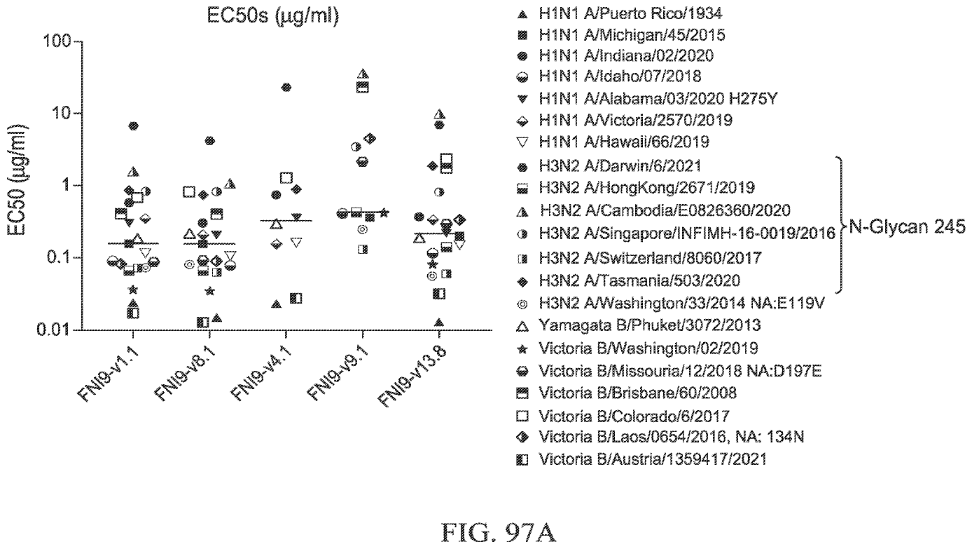

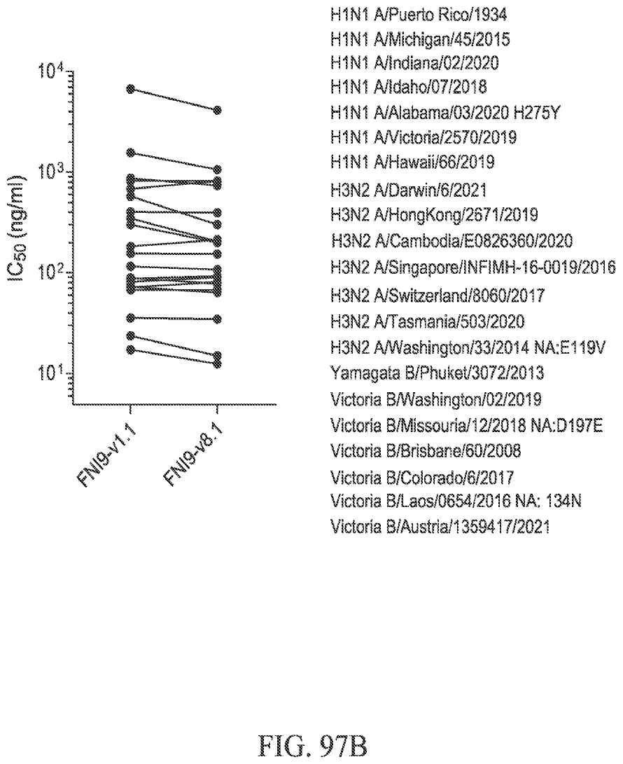

In vitro neutralizing activity of FNI9-v1.1, FNI9-v8.1, FNI9-v4.1, FNI9-v9.1, and FNI9-v13.8 was measured in a microneutralization assay (reported as EC50 in μg/ml) against a panel of seasonal IAVs (with and without glycosylation at position 245) and IBVs. Viruses were obtained from the International Reagent Resource (CDC, Atlanta, Georgia, USA) and Centre for Biological Reference Materials (NIBSC, Potters Bar, UK). Antibodies were serially diluted and added onto MDCK-LN cells previously infected with a defined viral input for 30 minutes. After 24 hours, cell infection was measured through viral nucleoprotein staining and analyzed via cell imaging. Dose-response curves were plotted to interpolate antibody's IC50 (half maximal inhibiting concentration) and IC90, which were reported as geometric mean of two independent experiments (FIGS. 97A-97B).

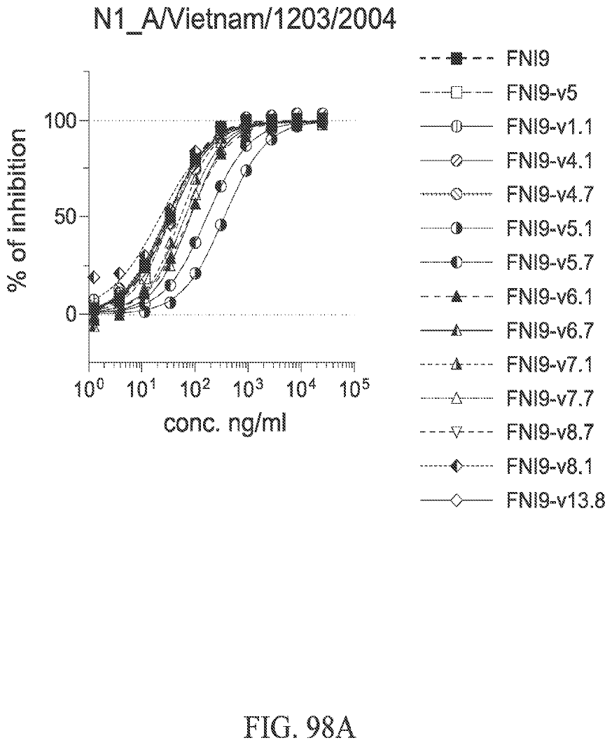

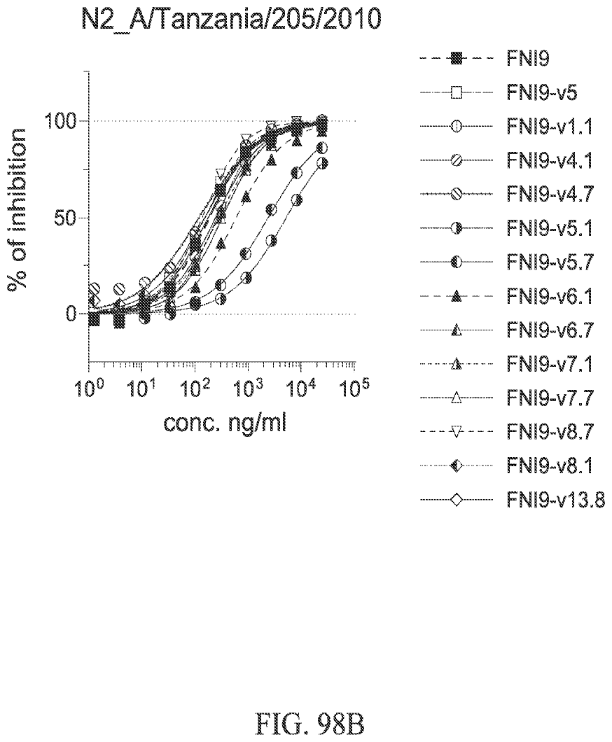

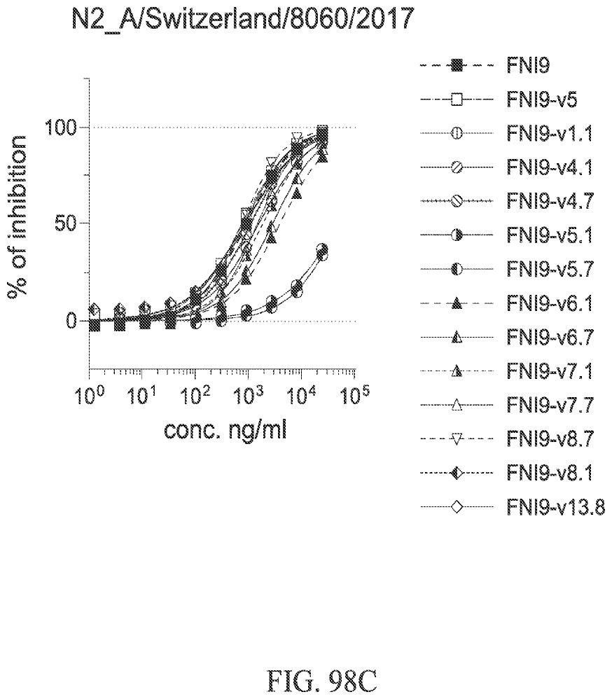

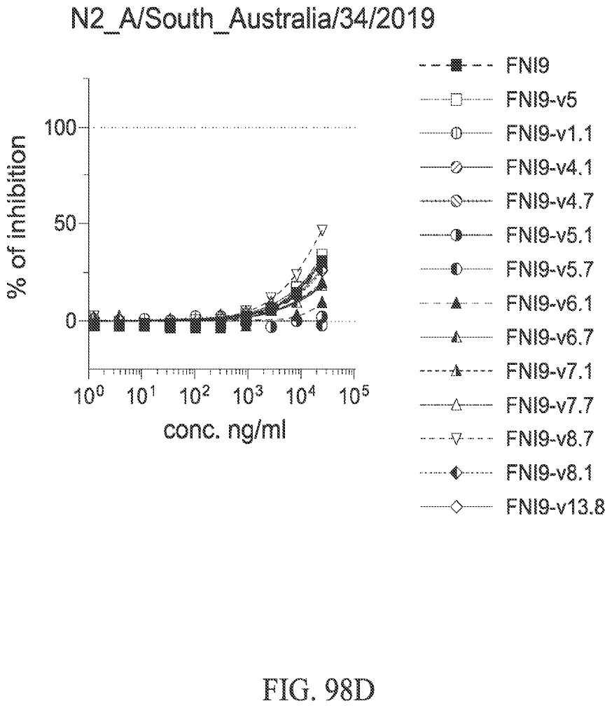

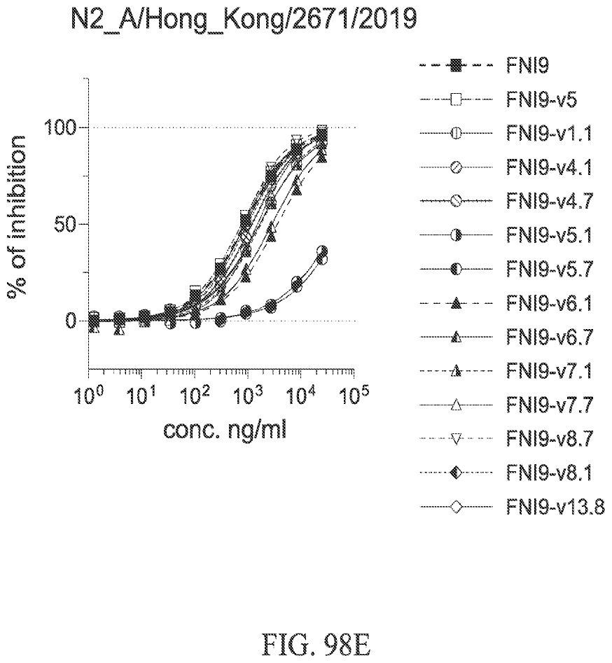

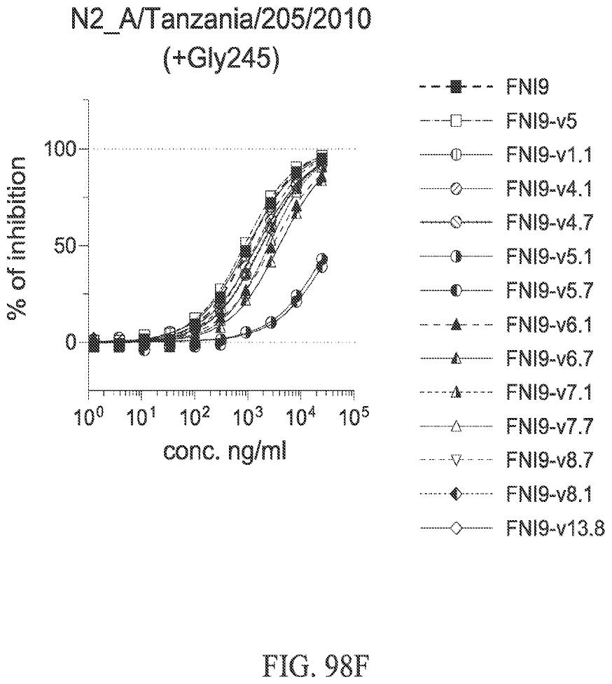

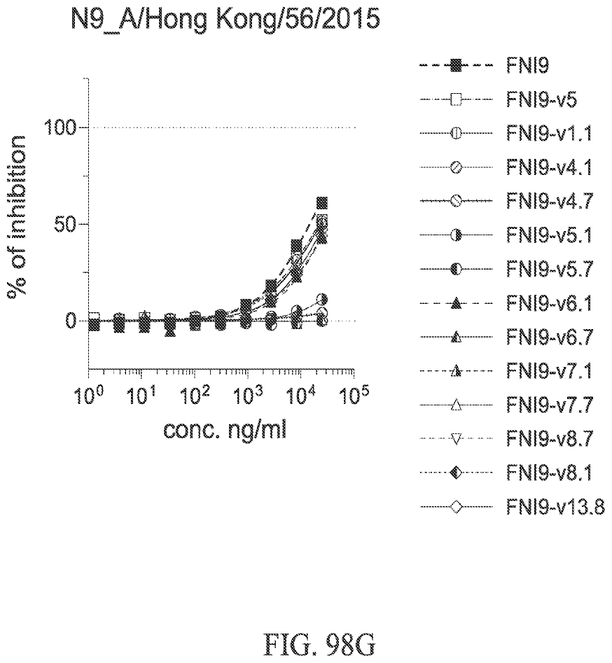

In vitro inhibition of sialidase activity by certain FNI9 variants against N1, N2, and N9 NAs was measured by MUNANA assay (FIGS. 98A-98G). FNI9-v4.1, FNI9-v4.7, FNI9-v5.1, FNI9-v5.7, FNI9-v6.1, FNI9-v6.7, FNI9-v7.1, FNI9-v7.7, FNI9-v8.7, FNI9-v8.1, and FNI9-v13.8 antibodies were tested. FNI9, FNI9-v1.1, and FNI9-v5 were tested as comparator antibodies. Inhibition of sialidase activity against N1 in A/Vietnam/1203/2004 (FIG. 98A), N2 in A/Tanzania/205/2010 (FIG. 98B), N2 in A/Switzerland/8060/2017 (FIG. 98C), N2 in A/South Australia/34/2019 (FIG. 98D), N2 in A/HongKong/2671/2019 (FIG. 98E), N2 in A/Tanzania/205/2010 (+Gly245) (FIG. 98F), and N9 in A/Hong Kong/56/2015 (FIG. 98G) is shown.

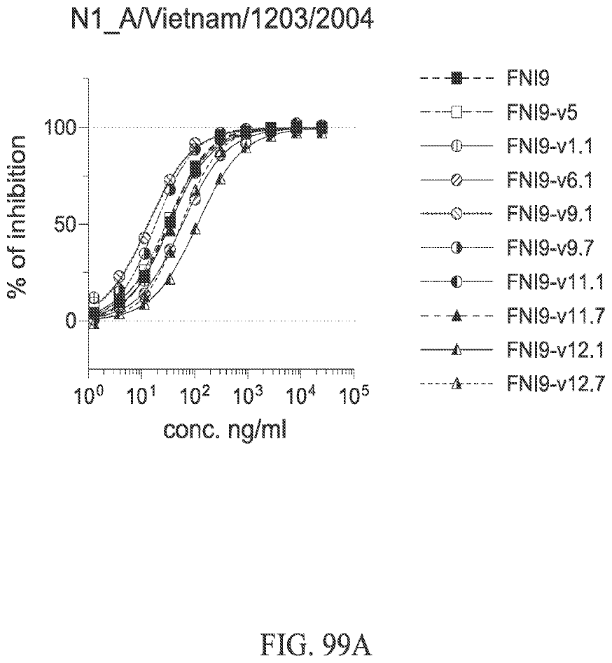

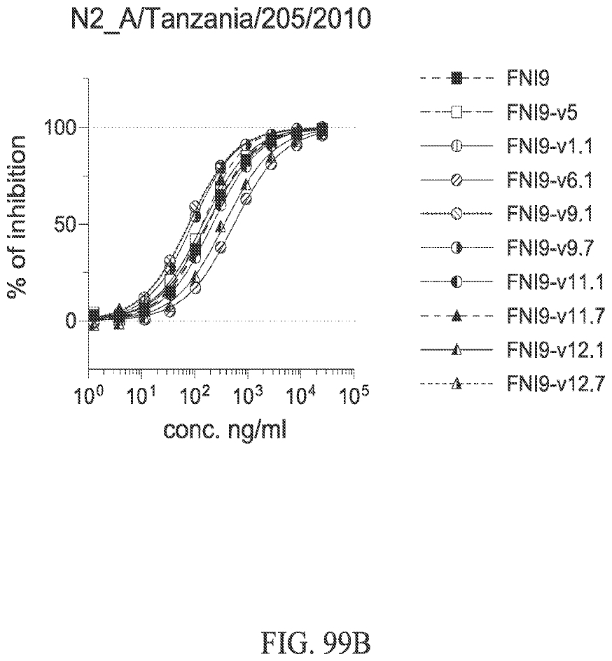

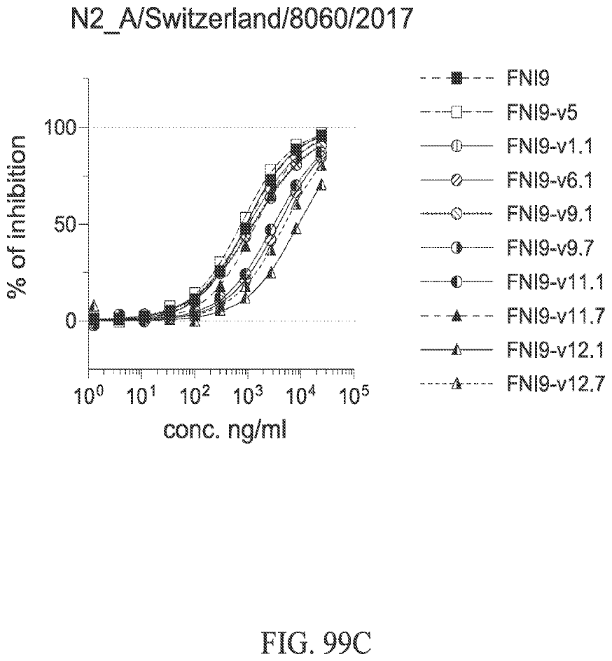

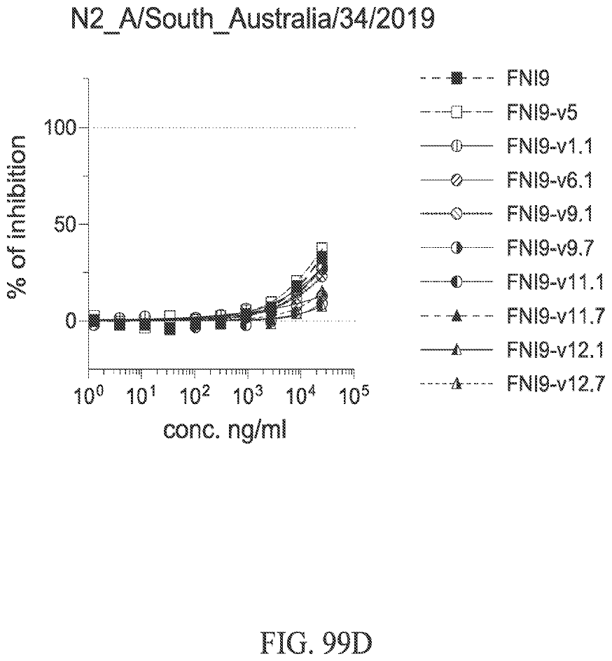

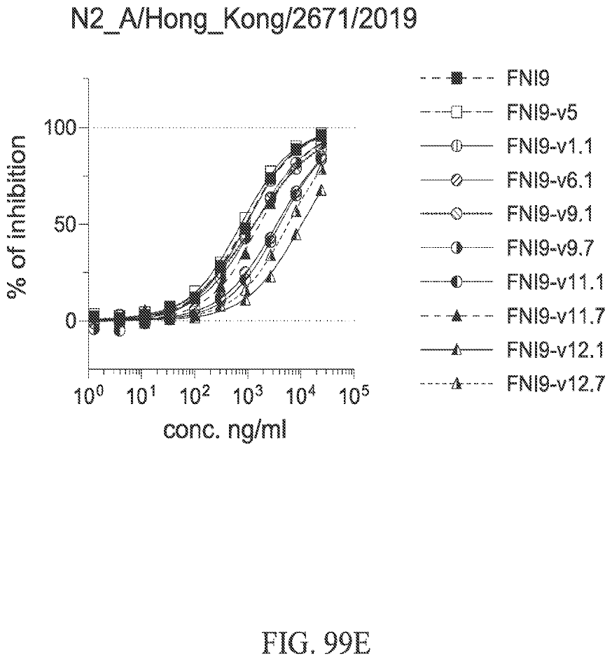

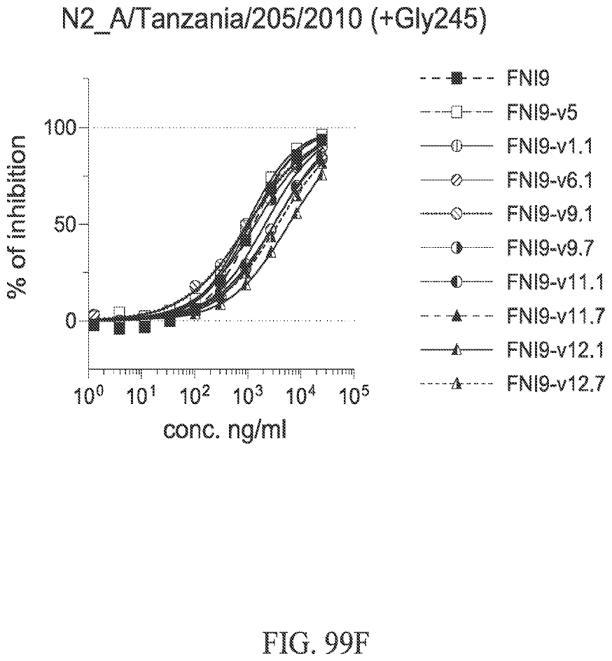

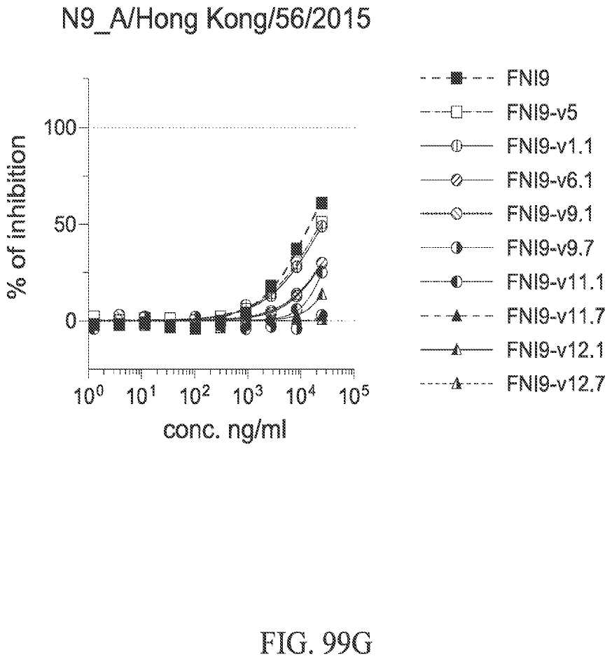

In vitro inhibition of sialidase activity by certain FNI9 variants against N1, N2, and N9 NAs was measured by MUNANA assay (FIGS. 99A-99G). FNI9-v6.1, FNI9-v9.1, FNI9-v9.7, FNI9-v11.1, FNI9-v11.7, FNI9-v12.1, and FNI9-v12.7 antibodies were tested. FNI9, FNI9-v1.1, and FNI9-v5 were tested as comparator antibodies. Inhibition of sialidase activity against N1 in A/Vietnam/1203/2004 (FIG. 99A), N2 in A/Tanzania/205/2010 (FIG. 99B), N2 in A/Switzerland/8060/2017 (FIG. 99C), N2 in A/South Australia/34/2019 (FIG. 99D), N2 in A/HongKong/2671/2019 (FIG. 99E), N2 in A/Tanzania/205/2010 (+Gly245) (FIG. 99F), and N9 in A/Hong Kong/56/2015 (FIG. 99G) is shown.

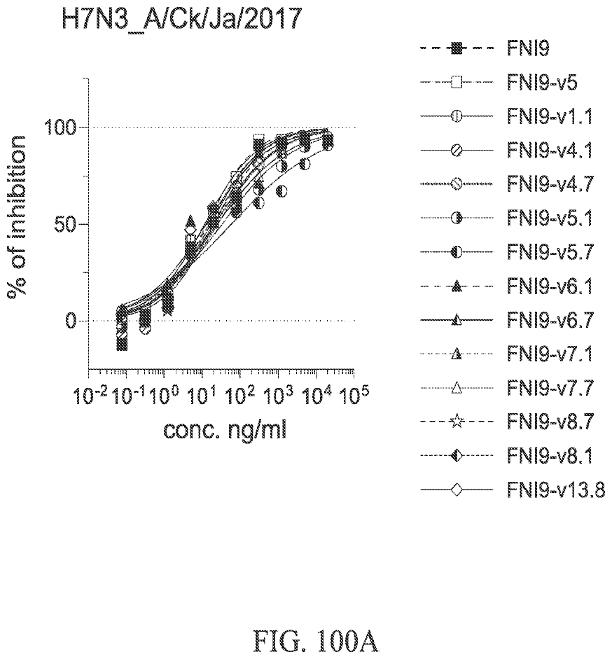

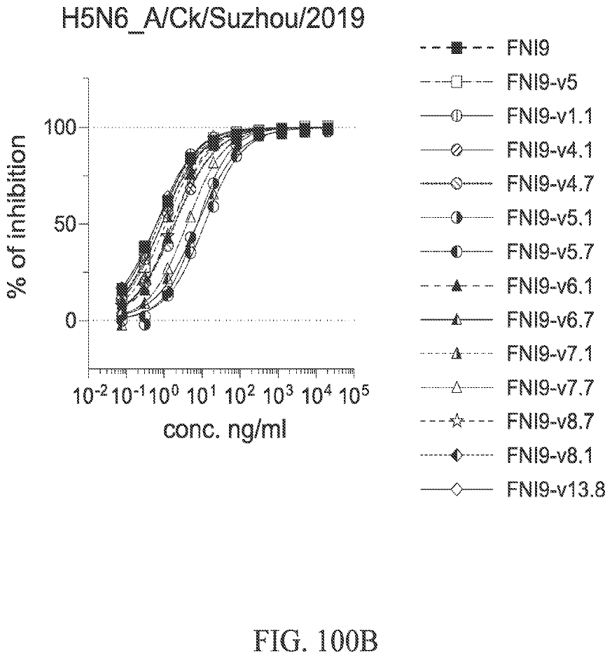

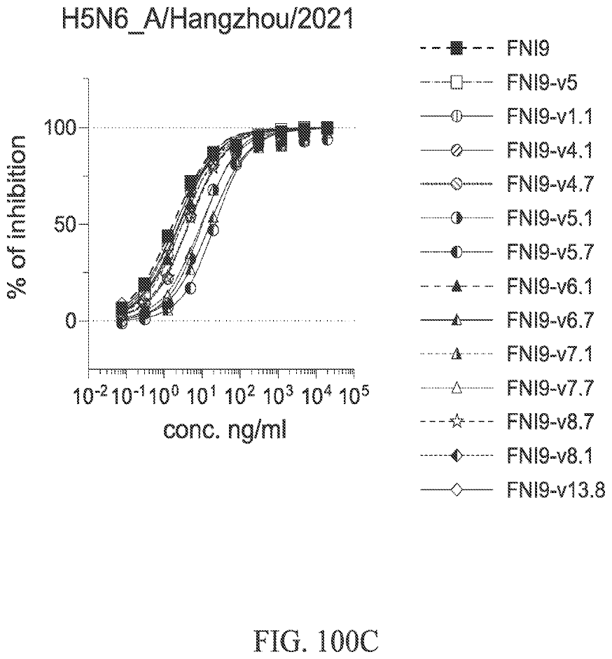

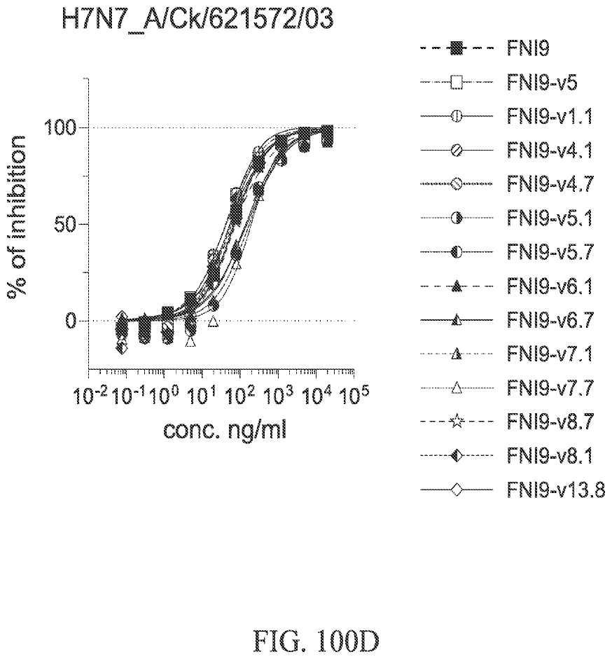

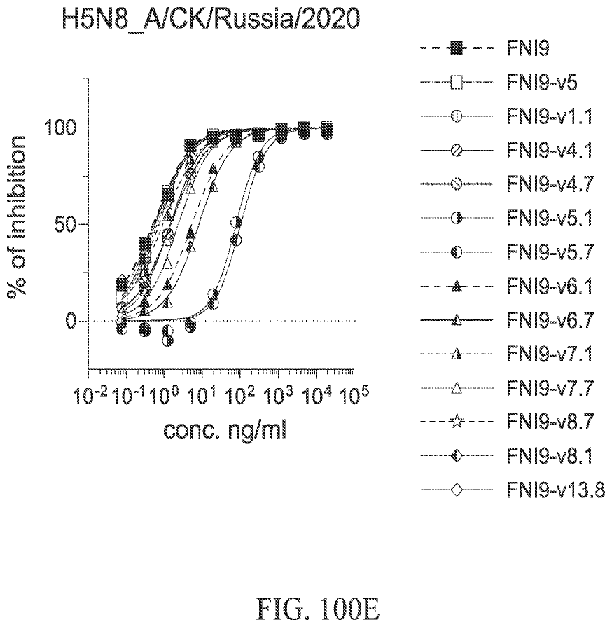

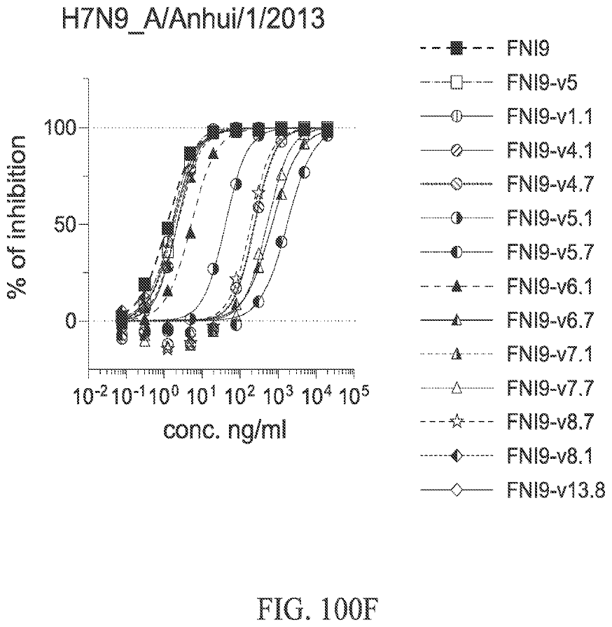

In vitro inhibition of sialidase activity by certain FNI9 variants against pseudovirus-derived NAs was measured by ELLA (FIGS. 100A-100F). FNI9-v4.1, FNI9-v4.7, FNI9-v5.1, FNI9-v5.7, FNI9-v6.1, FNI9-v6.7, FNI9-v7.1, FNI9-v7.7, FNI9-v8.7, FNI9-v8.1, and FNI9-v13.8 antibodies were tested. FNI9, FNI9-v1.1, and FNI9-v5 were tested as comparator antibodies. Inhibition of sialidase activity against H7N3 A/Ck/Ja/2017 (FIG. 100A), H5N6 A/Ck/Suzhou/2019 (FIG. 100B), H5N6 A/Hangzhou/2021 (FIG. 100C), H7N7 A/Ck/621572/03 (FIG. 100D), H5N8 A/Ck/Russia/2020 (FIG. 100E), and H7N9 A/Anhui/1/2013 (FIG. 100F) is shown.

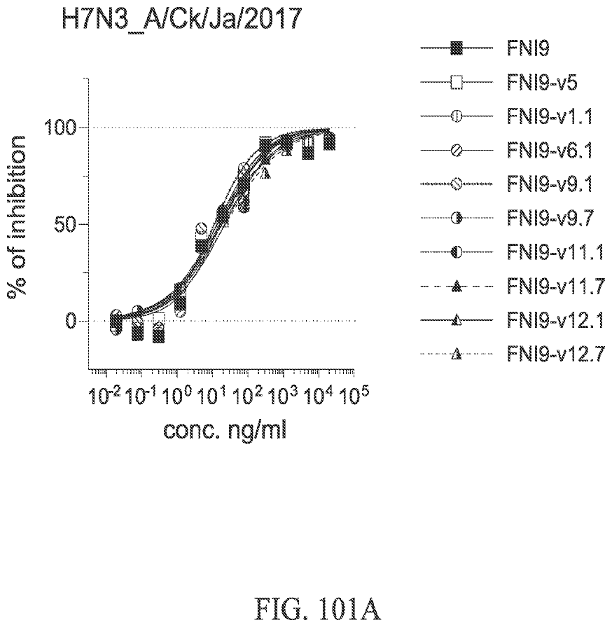

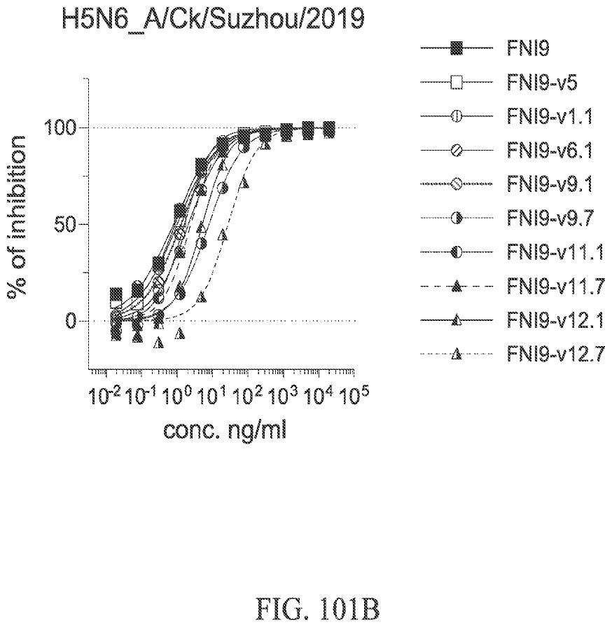

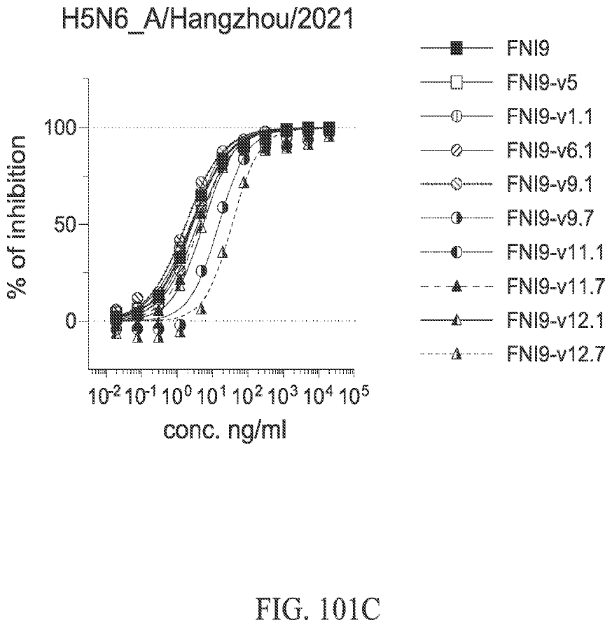

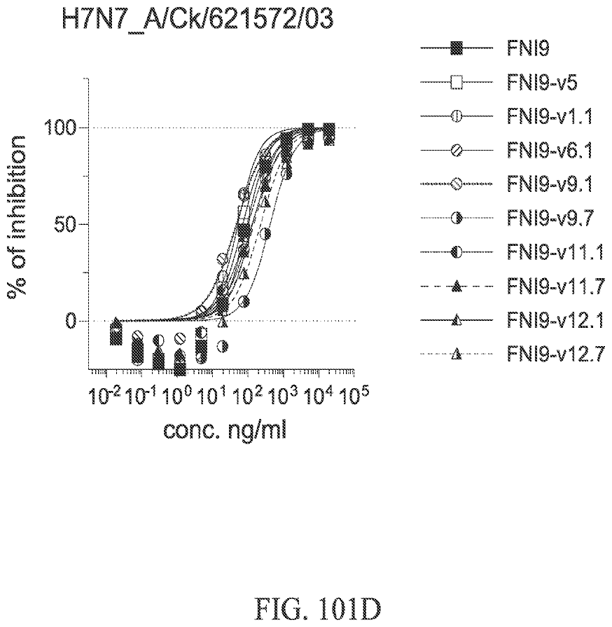

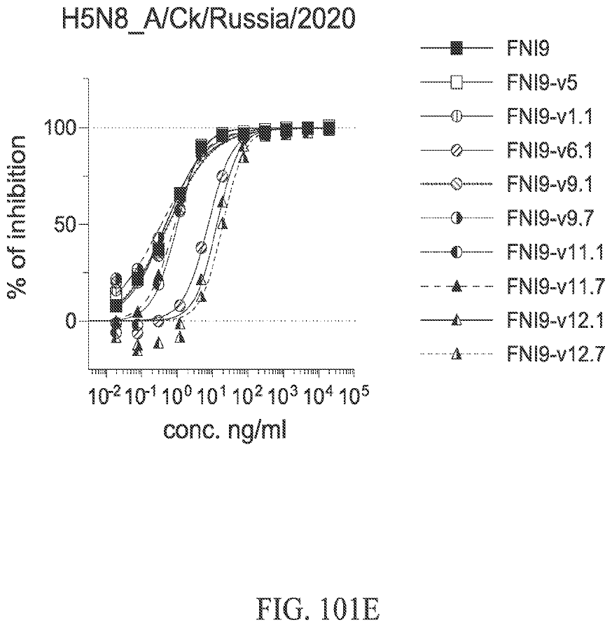

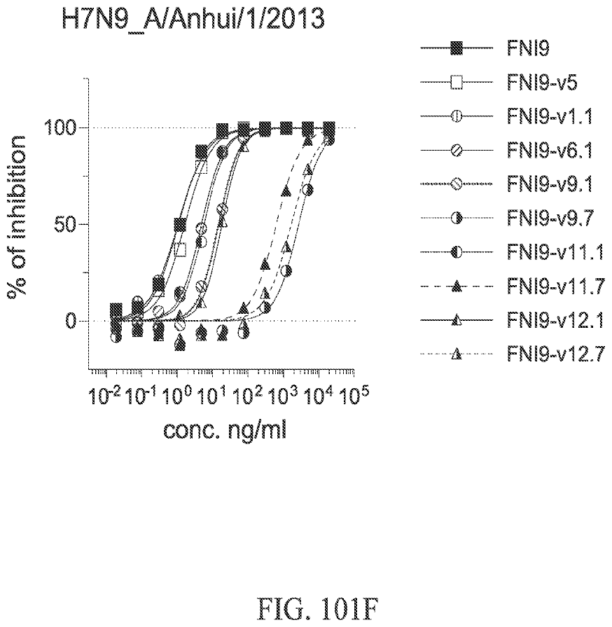

In vitro inhibition of sialidase activity by certain FNI9 variants against the indicated pseudovirus-derived NAs was measured by ELLA. FNI9-v6.1, FNI9-v9.1, FNI9-v9.7, FNI9-v11.1, FNI9-v11.7, FNI9-v12.1, and FNI9-v12.7 antibodies were tested. FNI9, FNI9-v1.1, and FNI9-v5 were tested as comparator antibodies. Inhibition of sialidase activity against H7N3 A/Ck/Ja/2017 (FIG. 101A) H5N6 A/Ck/Suzhou/2019 (FIG. 101B) H5N6 A/Hangzhou/2021 (FIG. 101C) H7N7 A/Ck/621572/03 (FIG. 101D) H5N8 A/Ck/Russia/2020 (FIG. 101E), and H7N9 A/Anhui/1/2013 (FIG. 101F) is shown.

### Example 17

### In Vitro Potency: FNI9, FNI19-v3, FNI17-v19, and FNI17-v19-LS

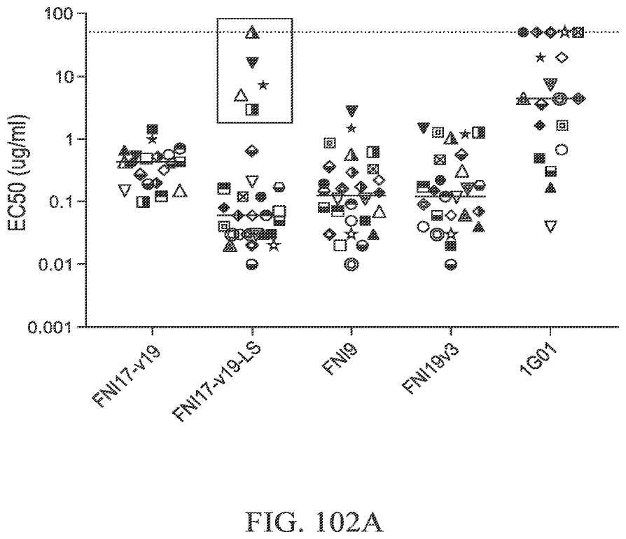

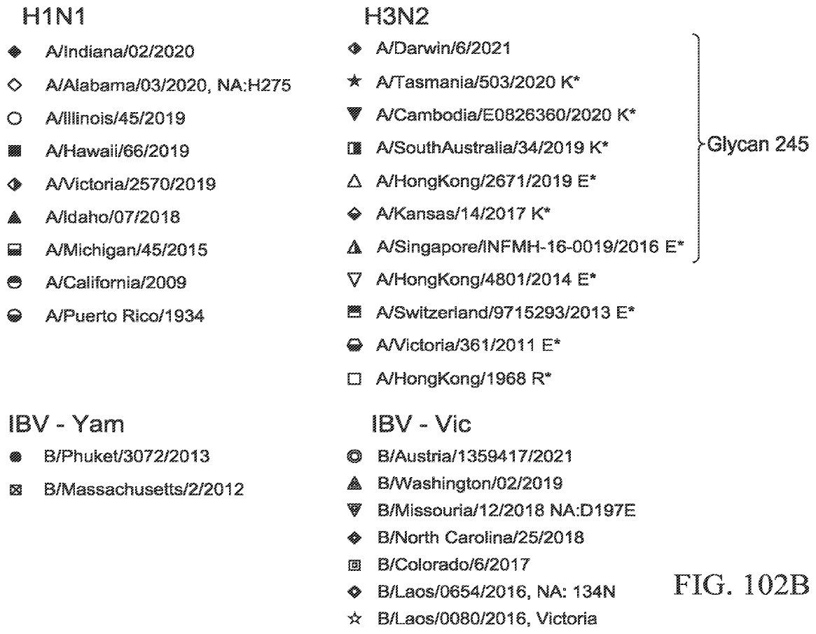

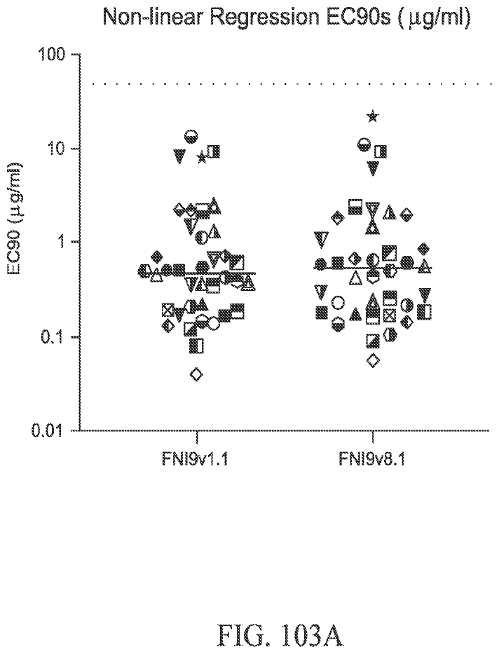

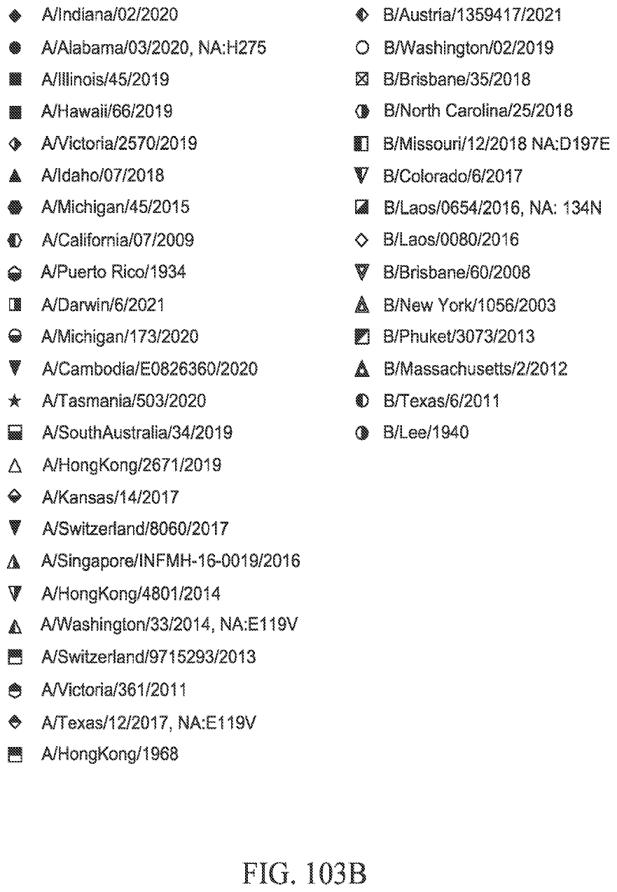

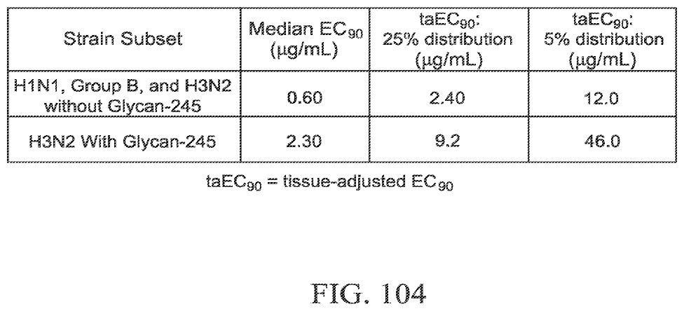

In vitro potency of FNI9, FNI19-v3, FNI17-v19, and FNI17-v19-LS, against group I (H1N1) IAV, group II (H3N2) IAV, and IBV NAs was measured by nucleoprotein (NP) staining. The results are shown in FIGS. 102A-102B and the rectangle indicates group II (H3N2) NAs harboring glycosylation at position 245. Neutralizing activity of a comparator antibody, 1G01, was also measured.

### Example 18

### In Vitro Potency Vs In Vivo Prophylactic Activity of Certain FNI Antibodies

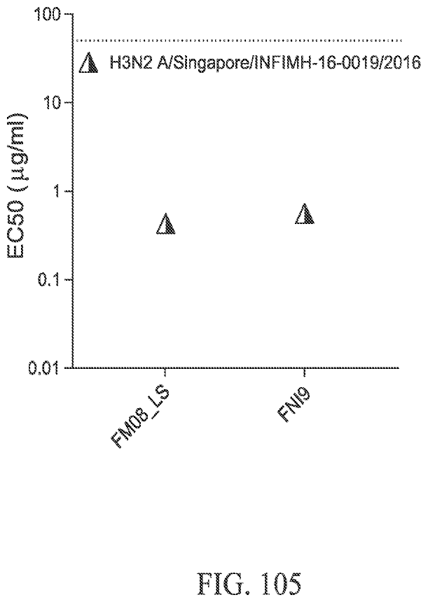

In vitro potency of FNI9 was tested. FIG. 105 shows in vitro neutralizing activity of FNI9 against H3N2 A/Singapore/INFIMH-16-0019/2016. Neutralizing activity of an anti-HA comparator antibody, FM08-LS, was also measured.

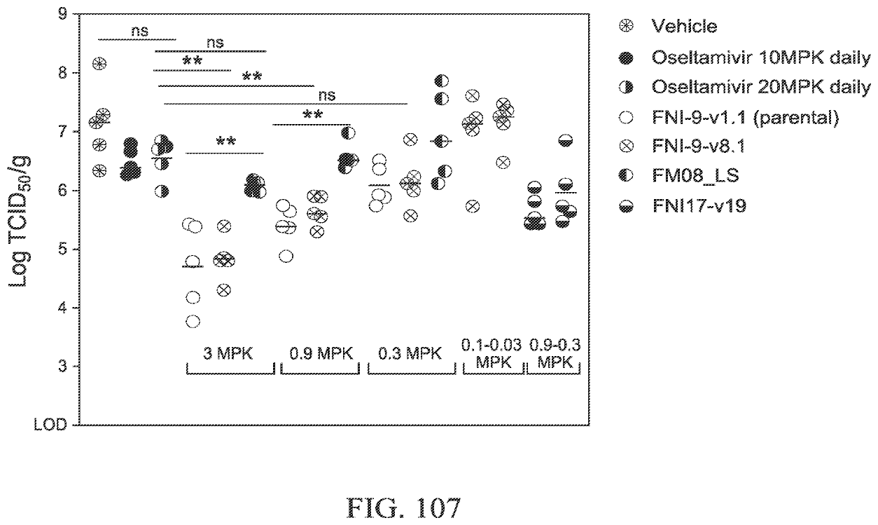

Prophylactic activity of FNI9, FNI9-v8.1, and FNI17-v19 were evaluated in a murine BALB/c model of H3N2 IAV infection. Briefly, BALB/c mice were treated with FNI9-v8.1, FNI9, or FNI17-v19 at 3 mg/kg, 0.9 mg/kg, 0.3 mg/kg, 0.1 mg/kg, or 0.03 mg/kg prior to infection with H3N2 A/Singapore/INFIMH-16-0019/2016. Viral titers in lung homogenates from tissue collected at four days post-infection are shown in FIG. 107. Viral titers were also measured in mice treated with OSE (10 mg/kg or 20 mg/kg) or an anti-HA comparator antibody, FM08_LS.

### Example 19

### Additional In Vitro Potency Vs In Vivo Prophylactic Activity Studies

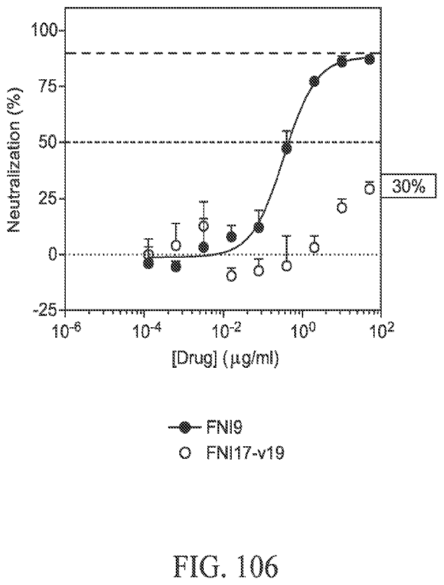

In vitro potency of FNI9 and FNI17-v19 was tested. FIG. 106 shows a dose response curve of in vitro neutralizing activity by FNI9 and FNI17-v19 against H3N2 A/Singapore/INFIMH-16-0019/2016.

Prophylactic activity of FNI9, FNI9-v8.1, and FNI17-v19 were evaluated in a murine BALB/c model of H3N2 IAV infection. Briefly, BALB/c mice were treated with FNI9-v8.1, FNI9, or FNI17-v19 at 3 mg/kg, 0.9 mg/kg, 0.3 mg/kg, 0.1 mg/kg, or 0.03 mg/kg prior to infection with H3N2 A/Singapore/INFIMH-16-0019/2016. Viral titers in lung homogenates from tissue collected at four days post-infection are shown in FIG. 107. Viral titers were also measured in mice treated with OSE (10 mg/kg or 20 mg/kg) or an anti-HA comparator antibody, FM08_LS.

### Example 20

### Prophylactic Activity: FNI9-v8.1

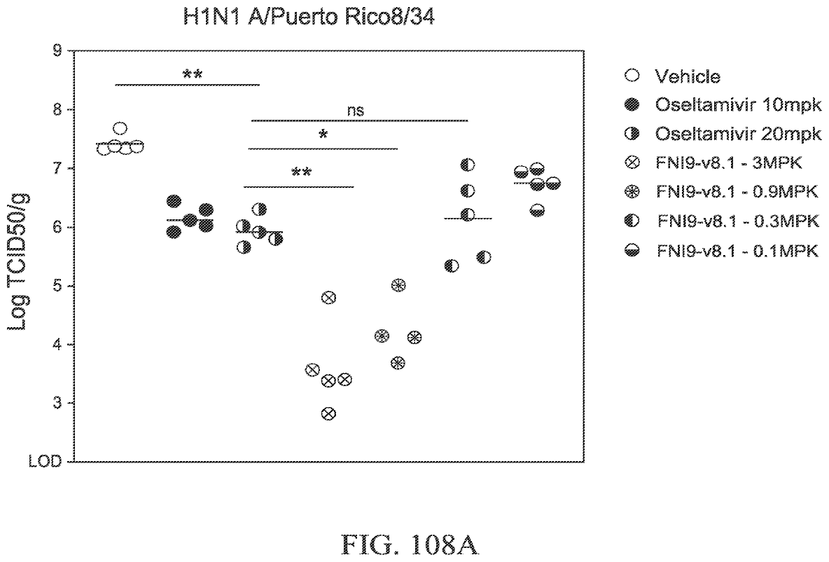

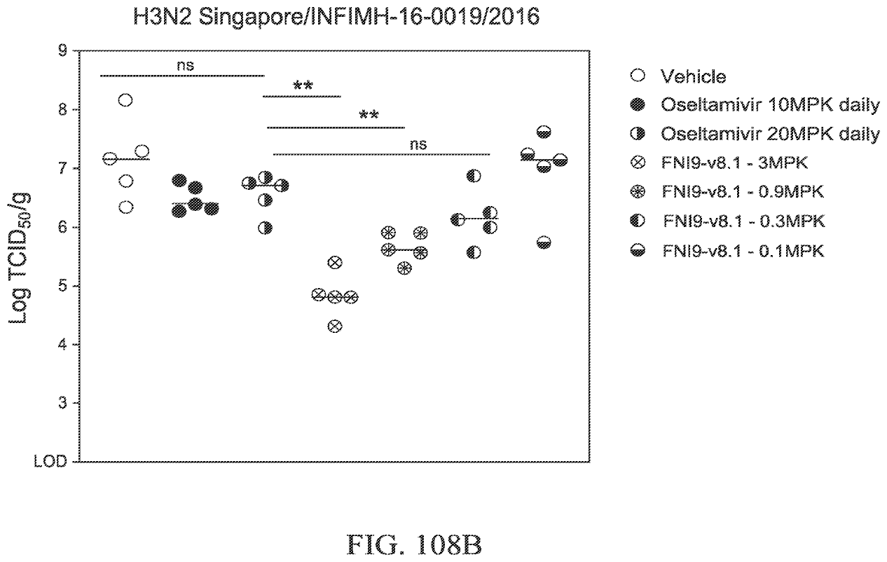

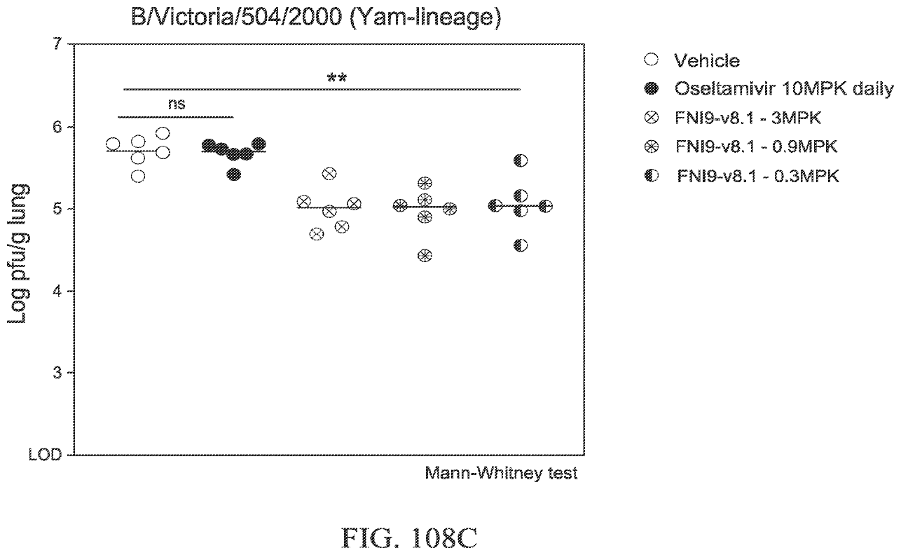

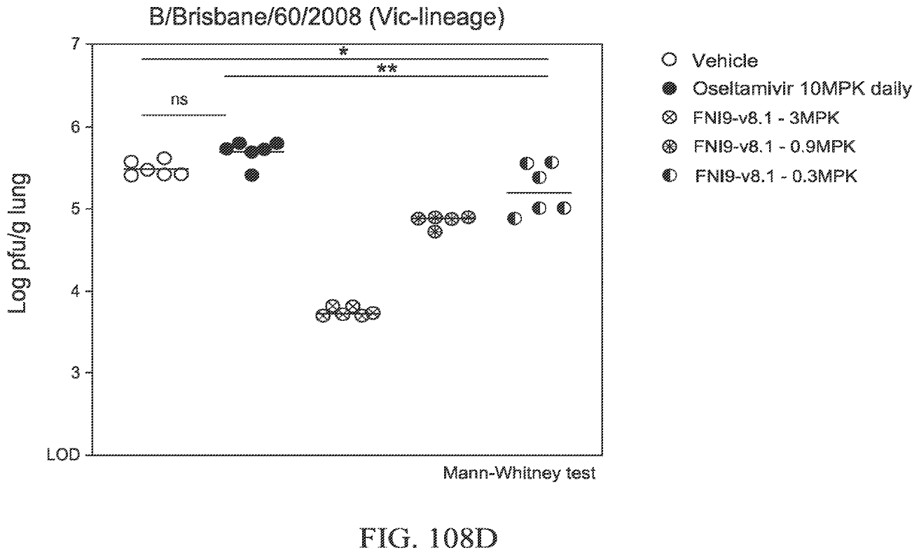

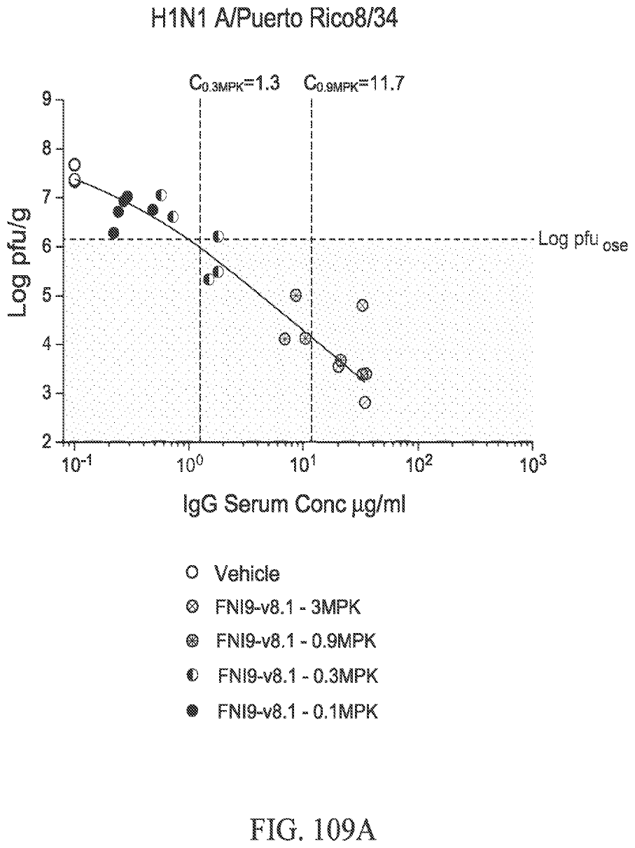

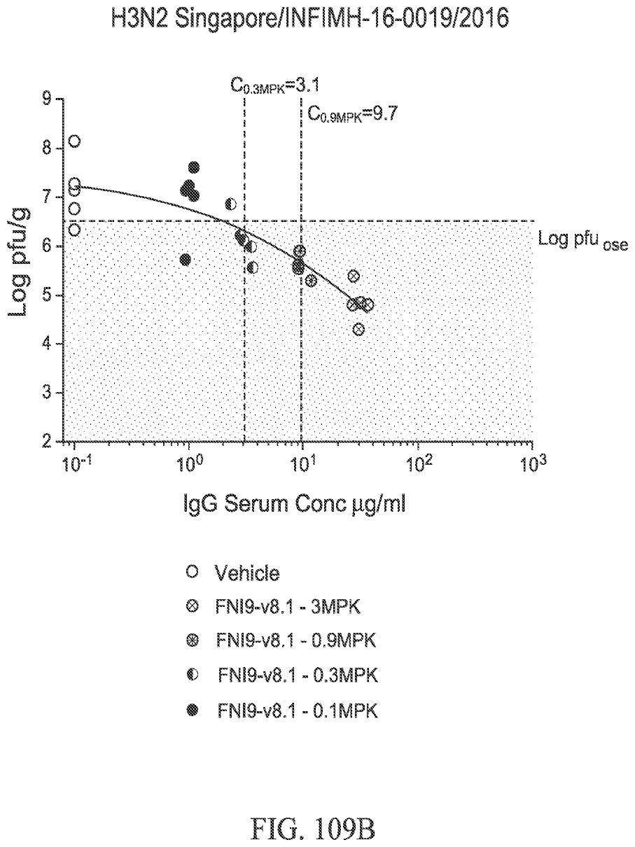

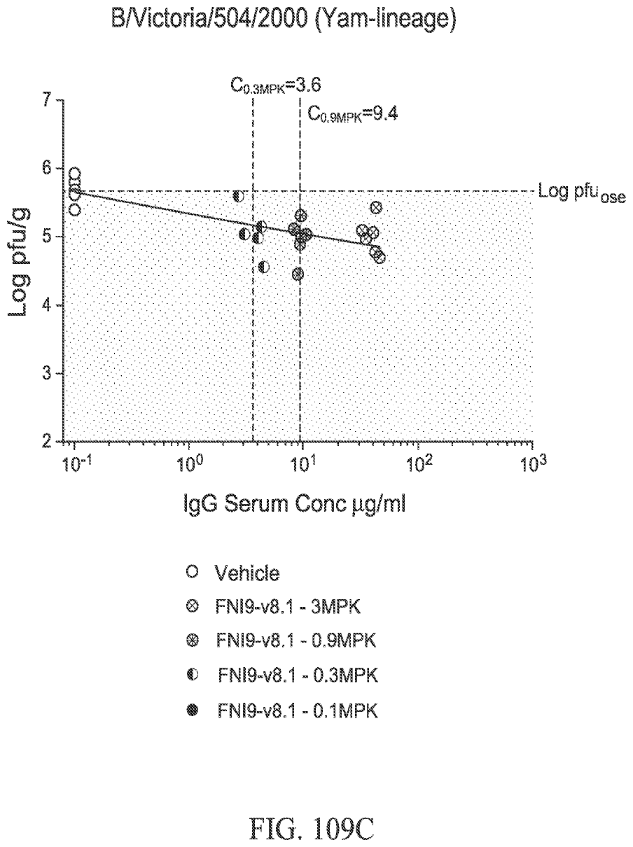

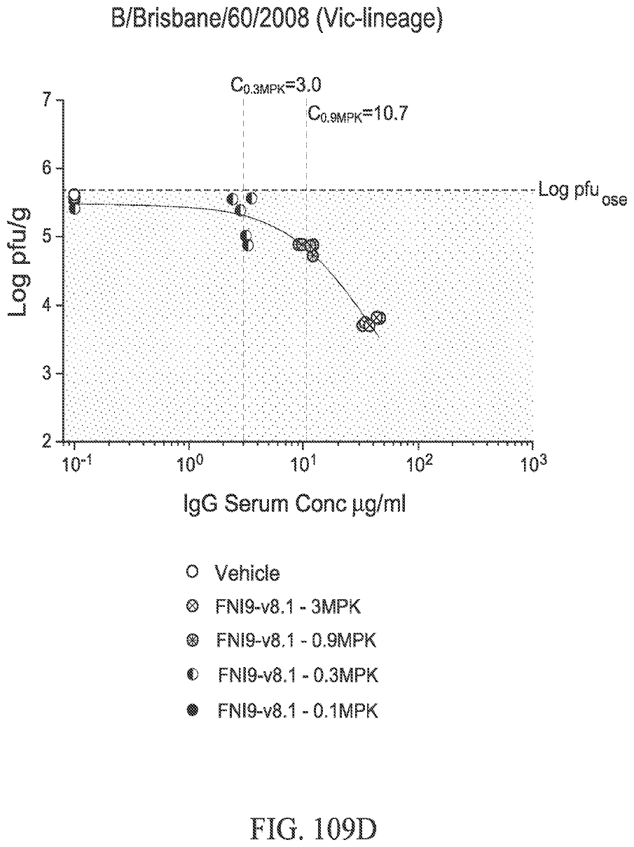

Prophylactic activity of FNI9-v8.1 was measured in a murine model of IAV and IBV infection. Briefly, BALB/c mice were treated with FNI9-v8.1 at 3 mg/kg, 0.9 mg/kg, 0.3 mg/kg, or 0.1 mg/kg prior to infection with H1N1 A/Puerto Rico/8/34 (FIG. 108A), H3N2 A/Singapore/INFIMH-16-0019/2016 (FIG. 108B), B/Victoria/504/2000 (Yamagata) (FIG. 108C), or B/Brisbane/60/2008 (Victoria) (FIG. 108D). Viral titers in lung homogenates from tissue collected at four days post-infection are shown in FIGS. 108A-108D. FIGS. 109A-109D show titer data reported as log plaque-forming units per gram tissue (Log pfu/g). Viral titers were also measured in mice treated with OSE (10 mg/kg or 20 mg/kg) or an anti-HA comparator antibody, FM08_LS.

### Example 21

### In Vitro Potency: FNI9-v8.1

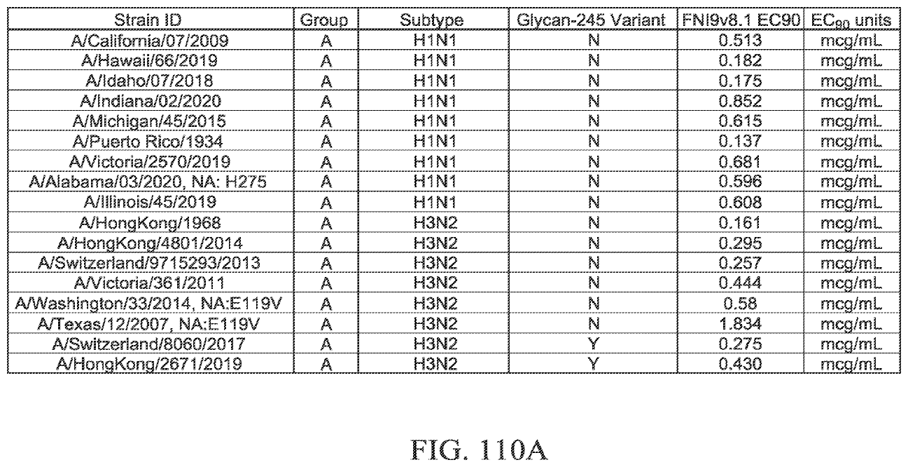

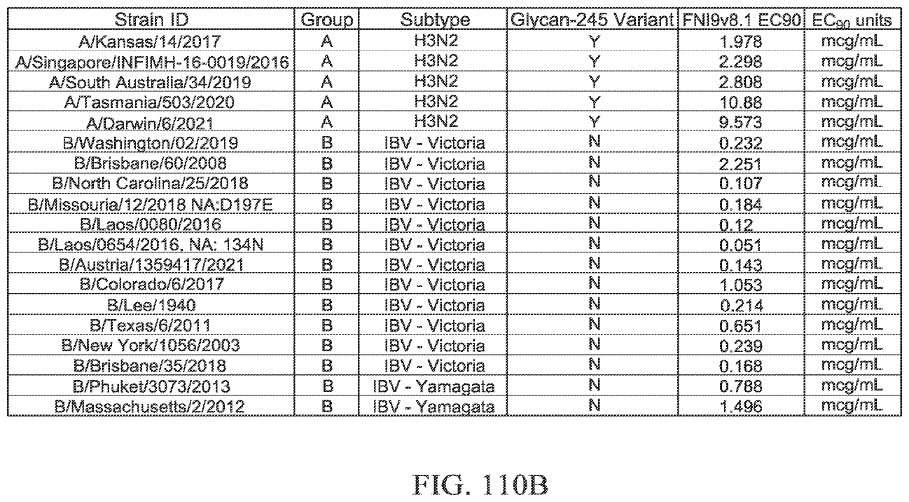

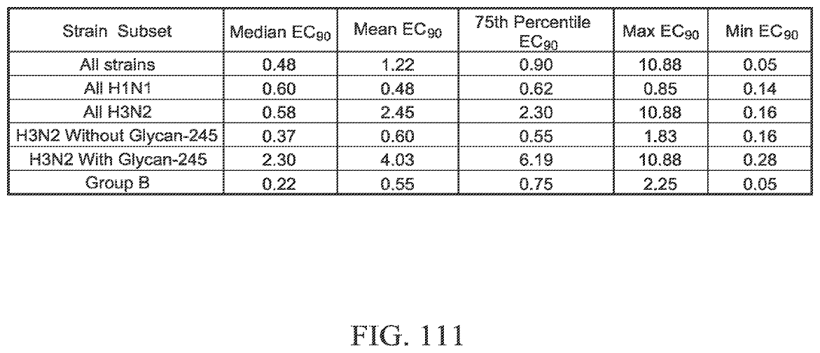

In vitro neutralization data by FNI9-v8.1 against a panel of H1N1 IAVs, H3N2 IAVs (with or without a glycosylation site at position 245), and IBVs was tested (FIGS. 110A and 110B).

A summary of in vitro neutralization statistics for FNI9-v8.1 against all strains, H1N1 IAVs, H3N2 IAVs, H3N2 IAVs (without a glycosylation site at position 245), H3N2 IAVs (with a glycosylation site at position 245), and IBVs is shown in FIG. 111.

### Example 22

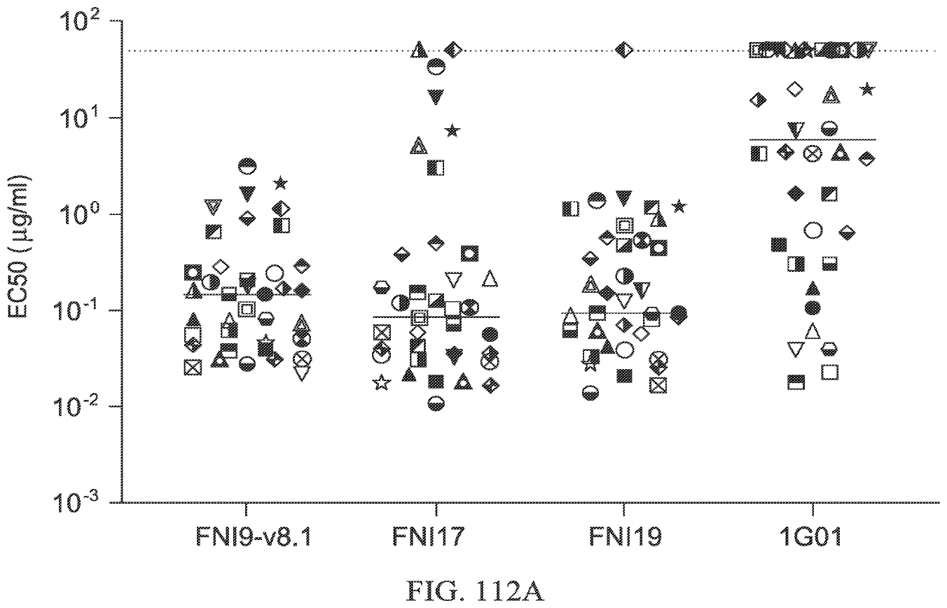

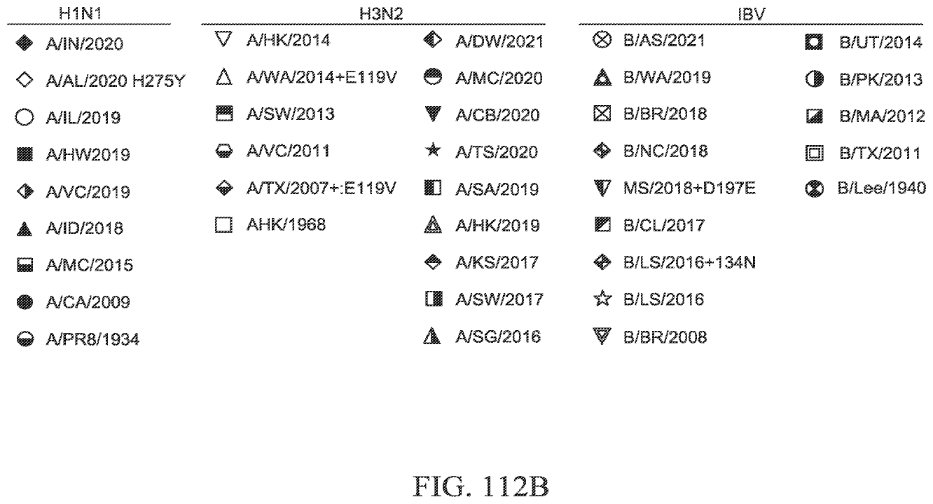

In Vitro Potency: FNI9-v8.1, FNI17, and FNI19 In vitro potency of FNI9-v8.1, FNI17, and FNI19, against group I (H1N1) IAV, group II (H3N2) IAV, and IBV NAs was measured by nucleoprotein (NP) staining. The results are shown in FIGS. 112A-112B. Neutralizing activity of comparator antibody, 1G01, was also measured.

### Example 23

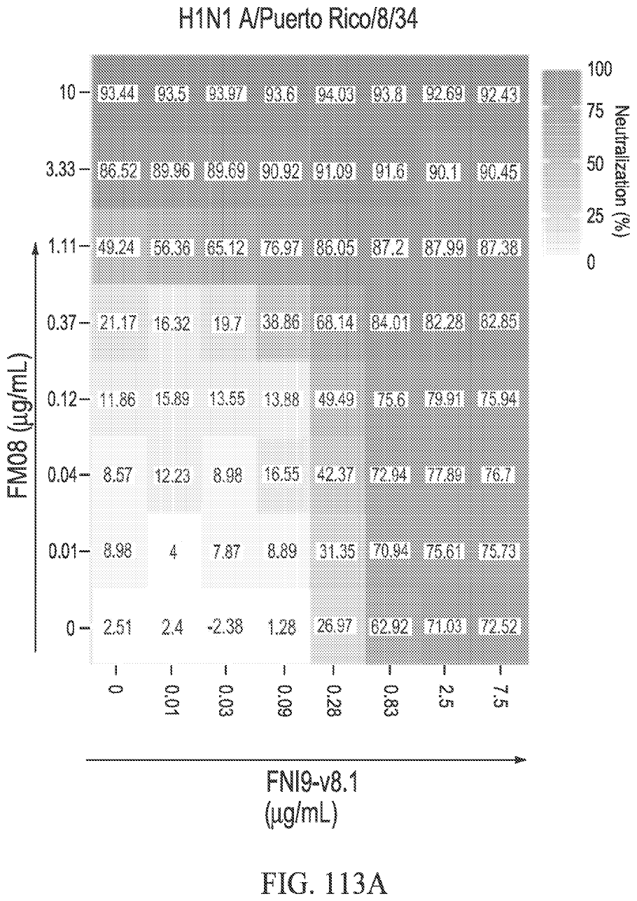

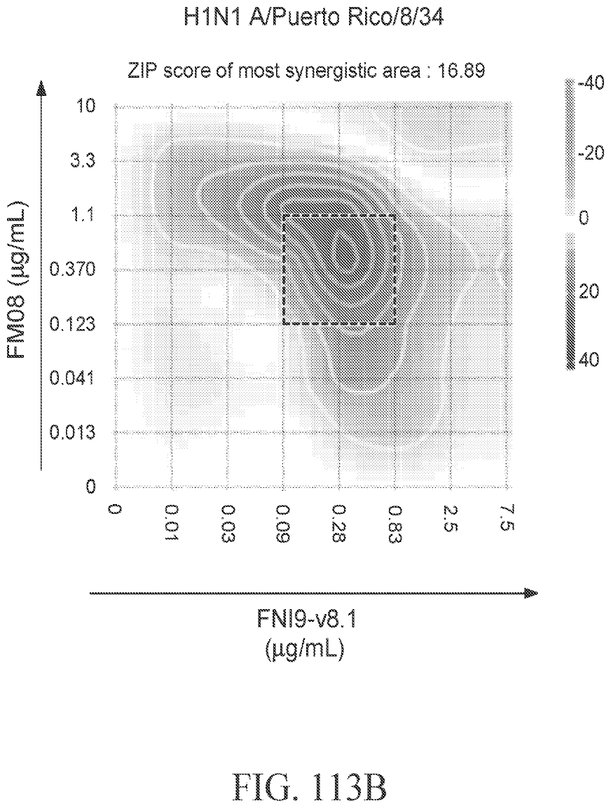

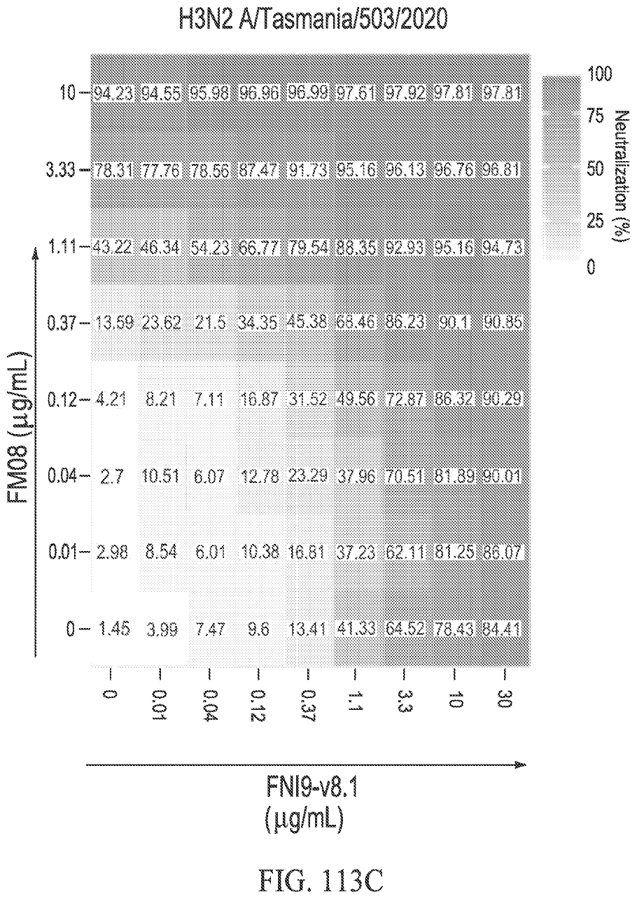

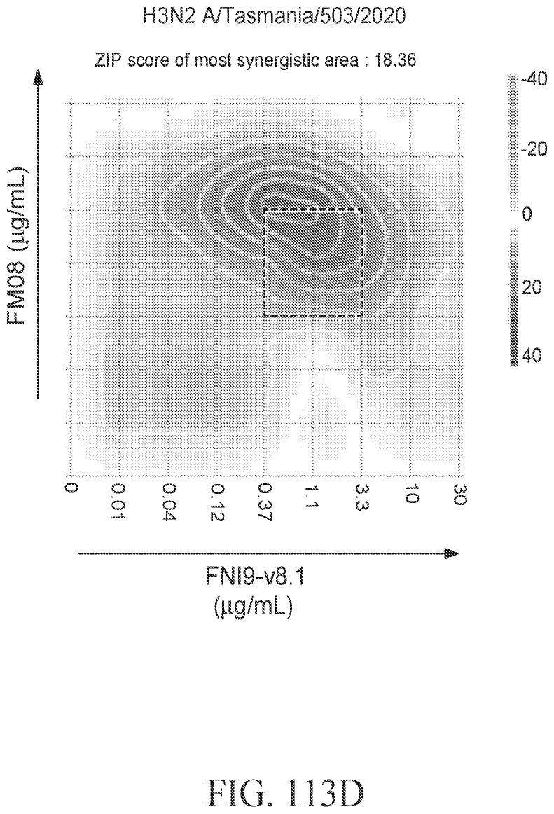

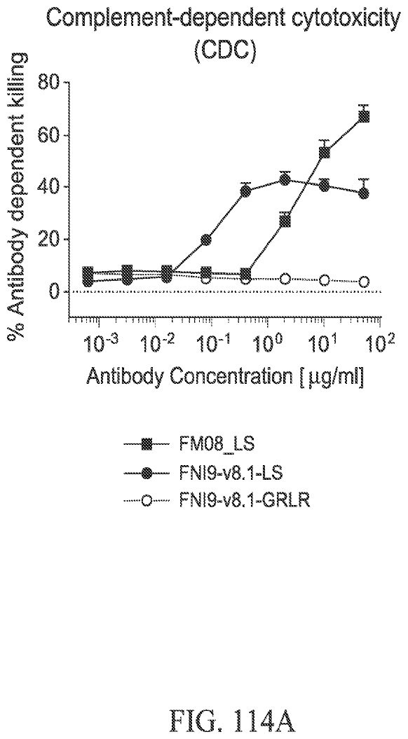

In Vitro Potency: Combination Activity of FNI9-v8.1 and FM08_Ls Combined in vitro neutralization activity of FNI9-v8.1 (anti-NA) and FM08 (anti-HA) was tested against H1N1 A/Puerto Rico/8/34 (FIGS. 113A and 113B) and H3N2 A/Tasmania/503/2020 (FIGS. 113C and 113D). Neutralization matrixes (reported in μg/ml) are shown in FIGS. 113A and 113C. Synergy plots are shown in FIGS. 113B and 113D.

### Example 24

### Antibody Effector Functions: FNI9-v8.1

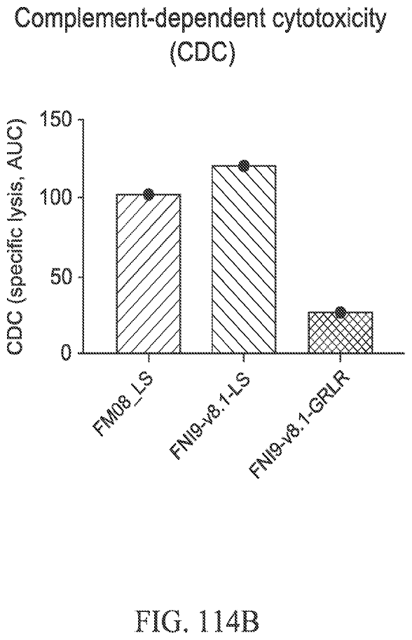

Antibody effector functions were evaluated. Complement-dependent cytotoxicity (CDC) mediated by FNI9-v8.1-LS on MDCK-LN cells infected with H1N1 A/Puerto Rico/8/34 in the presence of guinea pig complement was measured (FIGS. 114A-114B). CDC of an anti-HA comparator antibody, FM08_LS, and a Fc-silent negative control, FNI9-v8.1-GRLR, was also measured. CDC is reported as % antibody-dependent killing in FIG. 114A and area-under-the-curve of antibody-dependent killing in FIG. 114B.

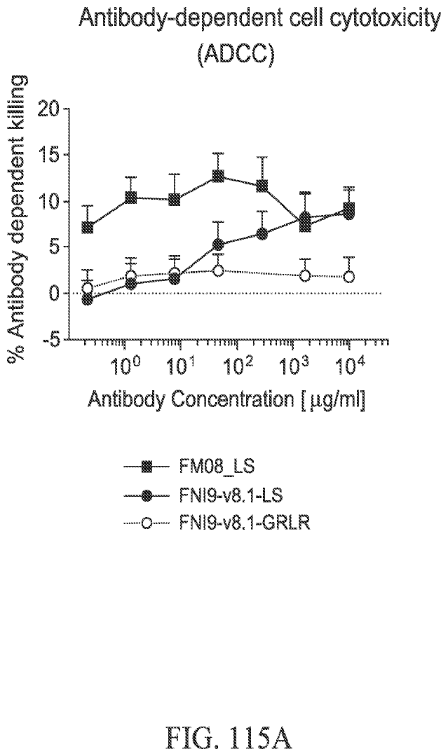

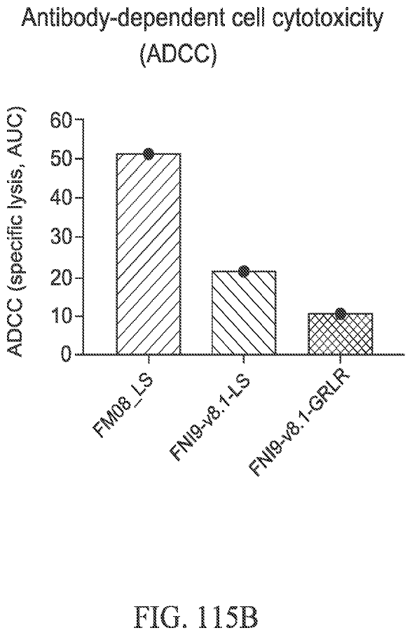

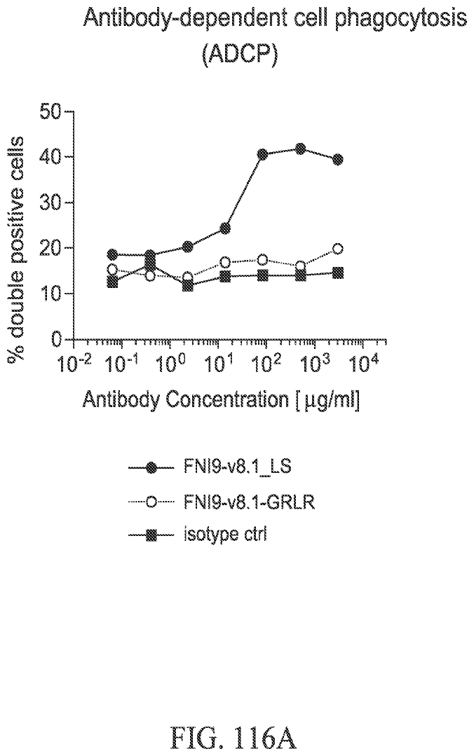

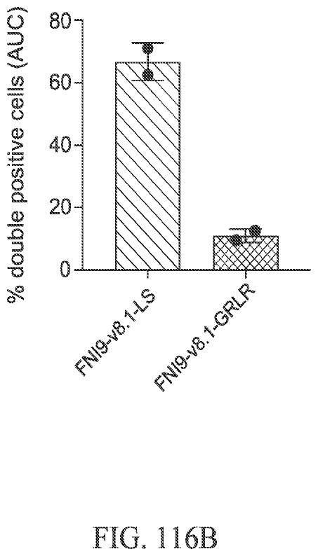

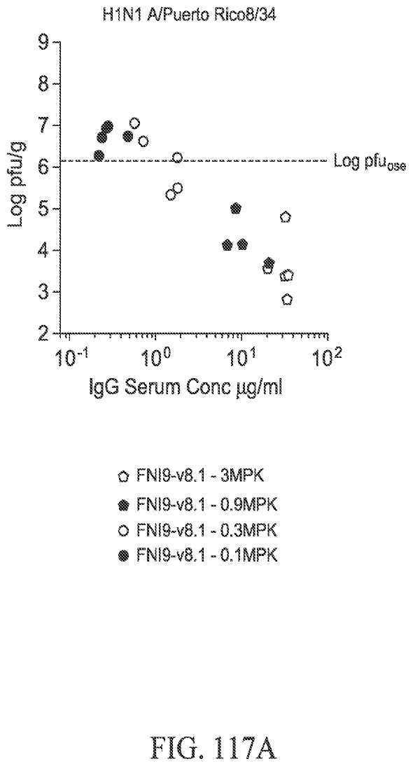

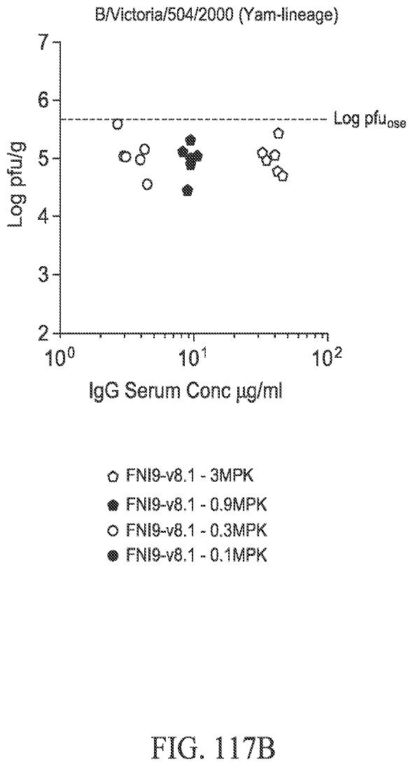

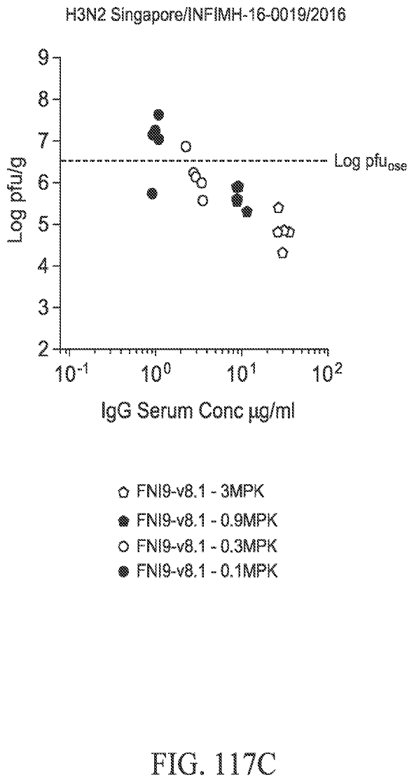

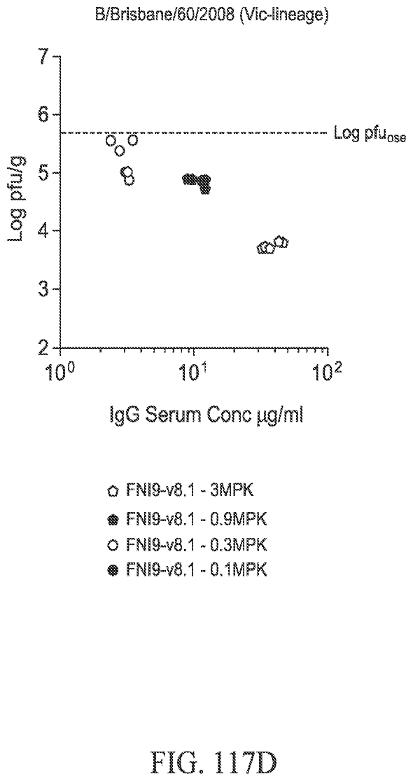

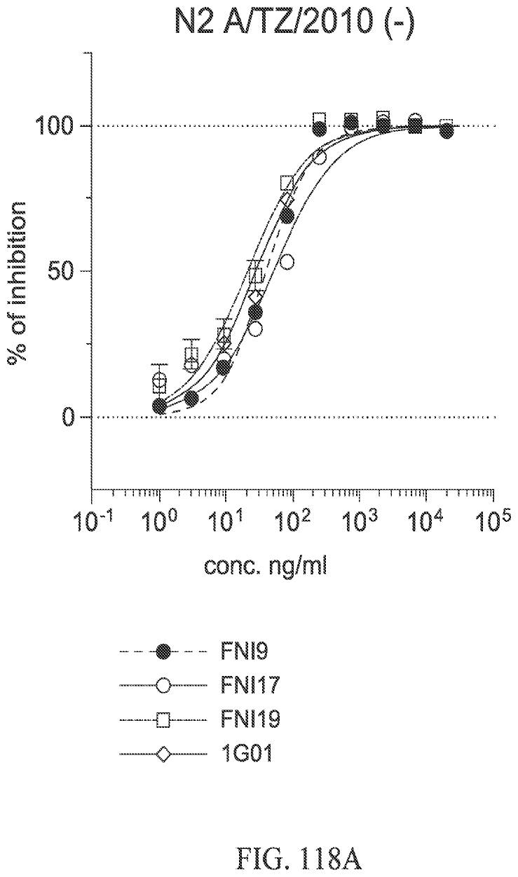

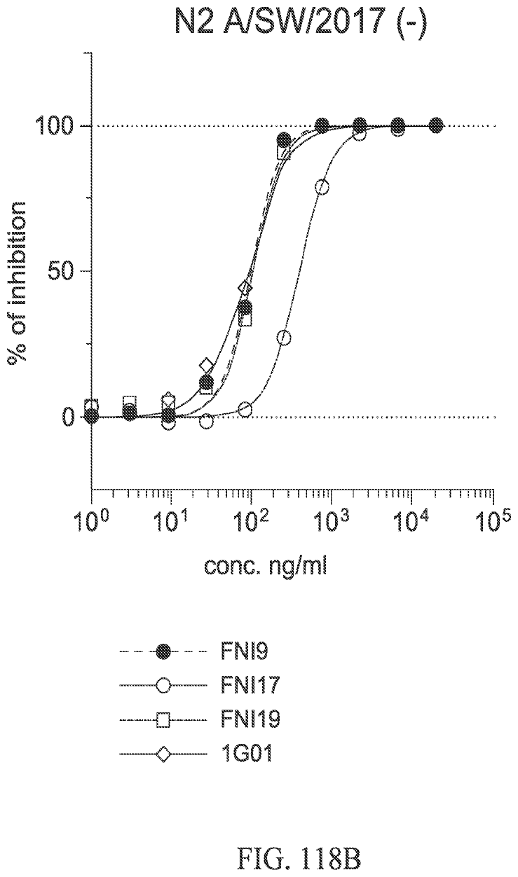

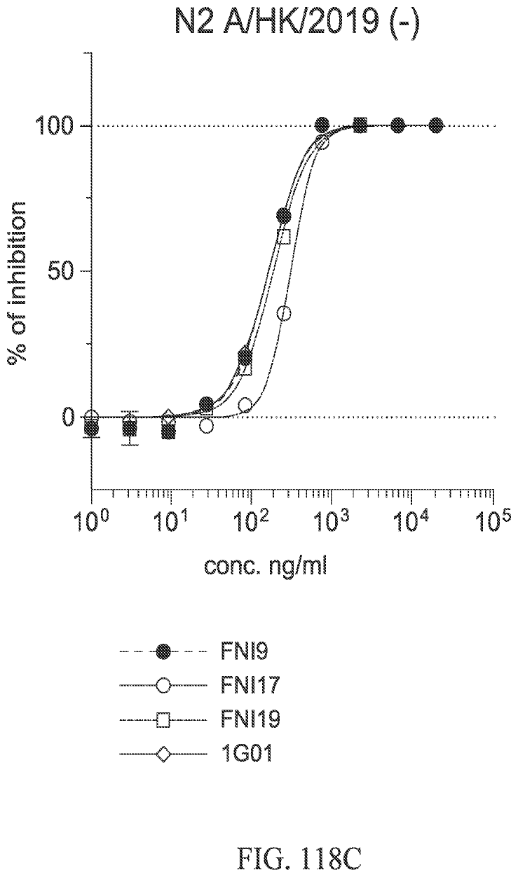

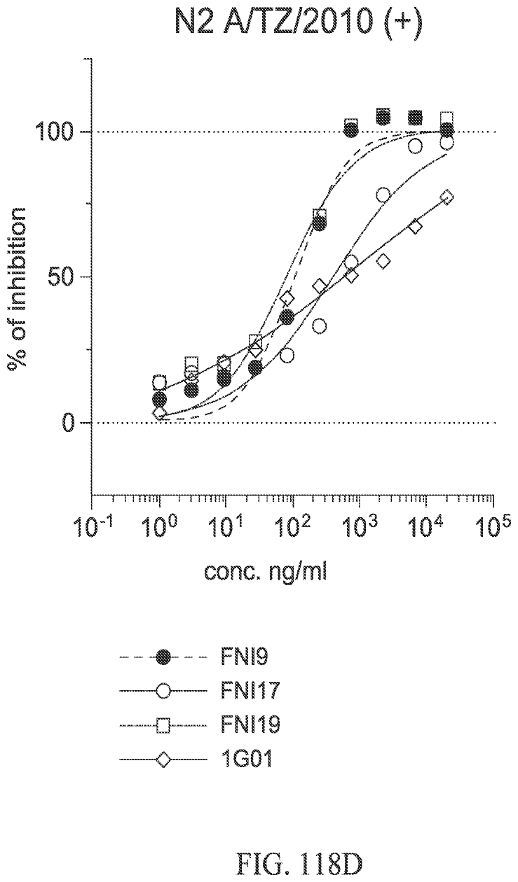

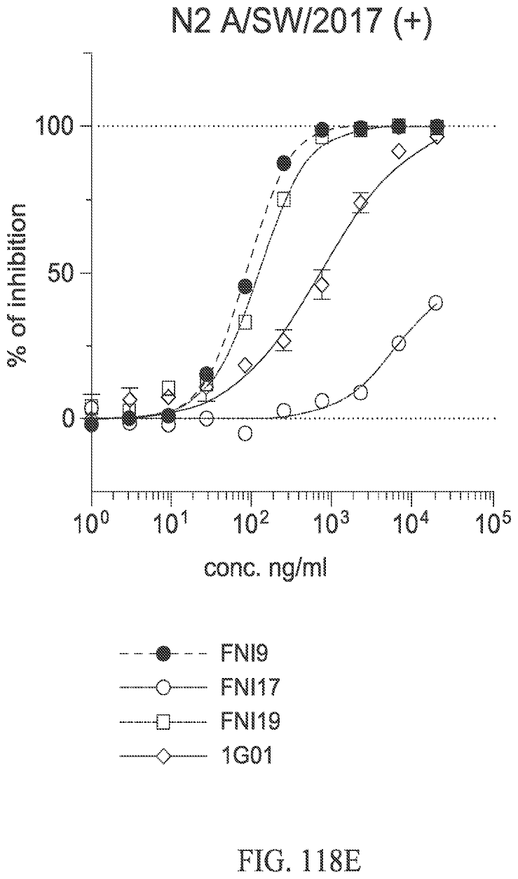

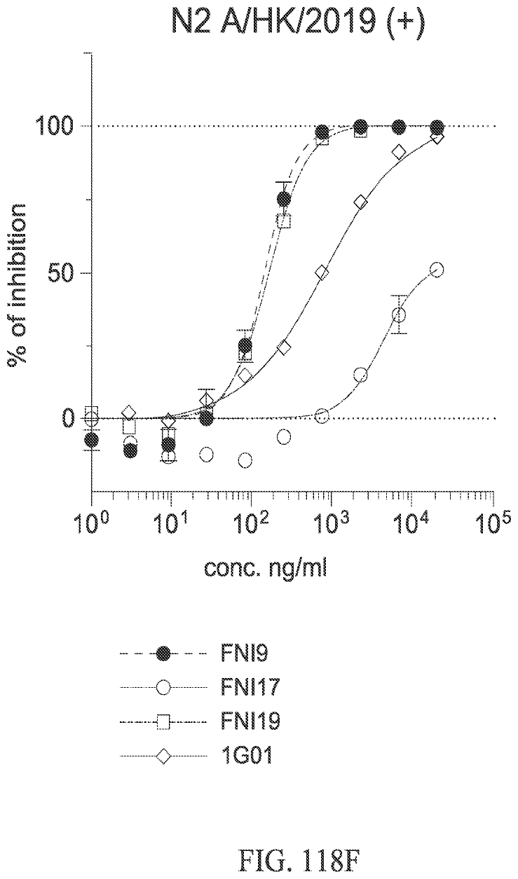

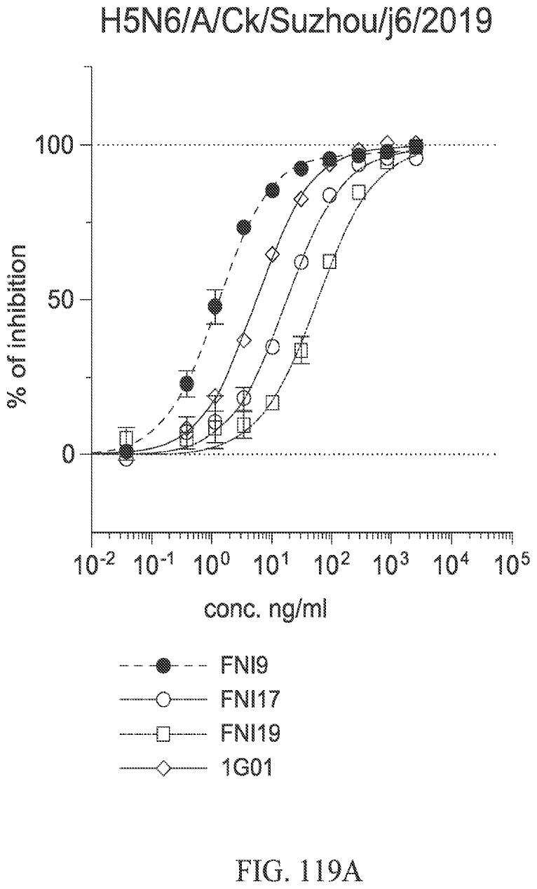

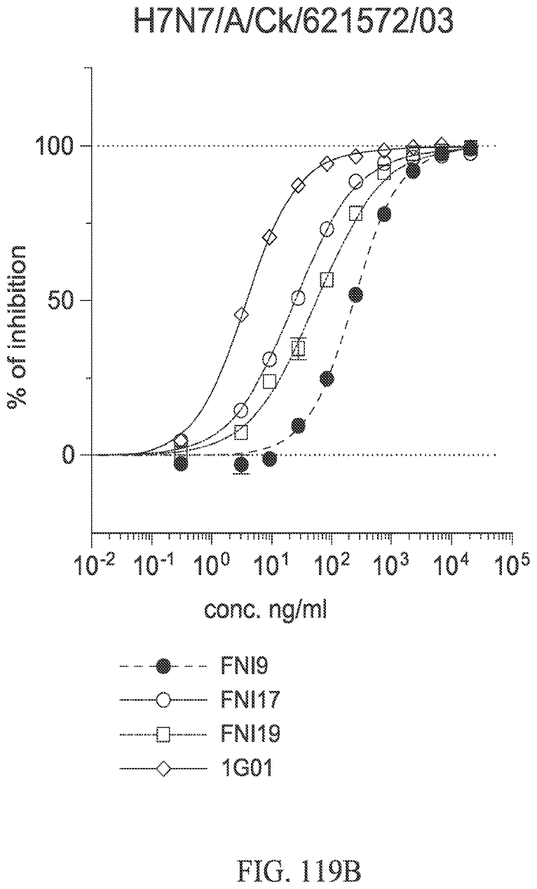

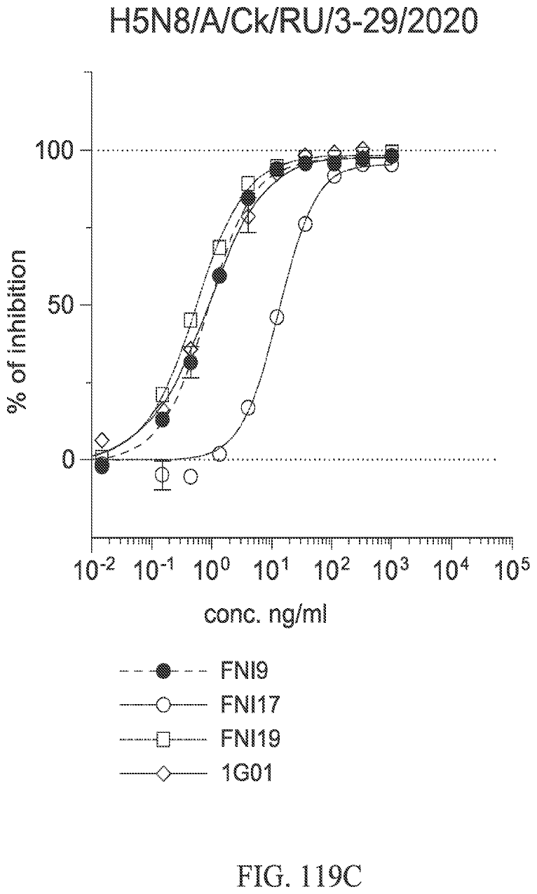

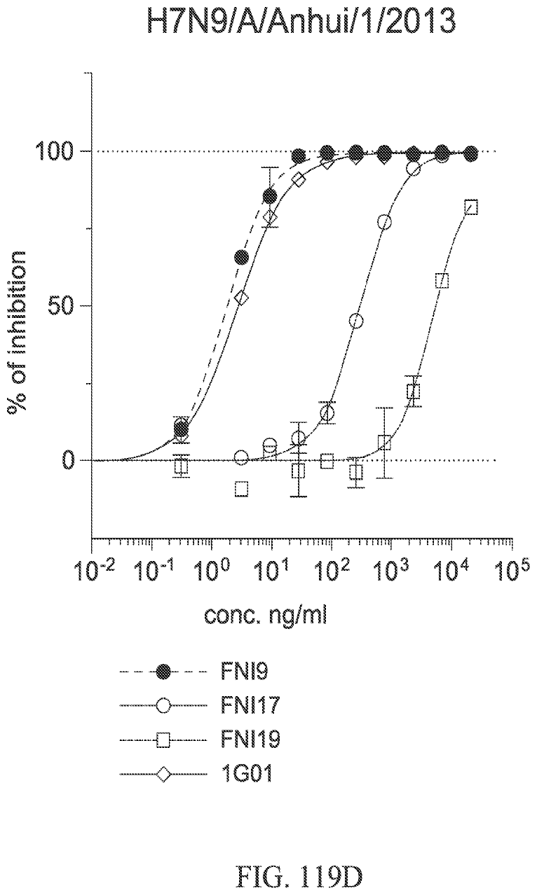

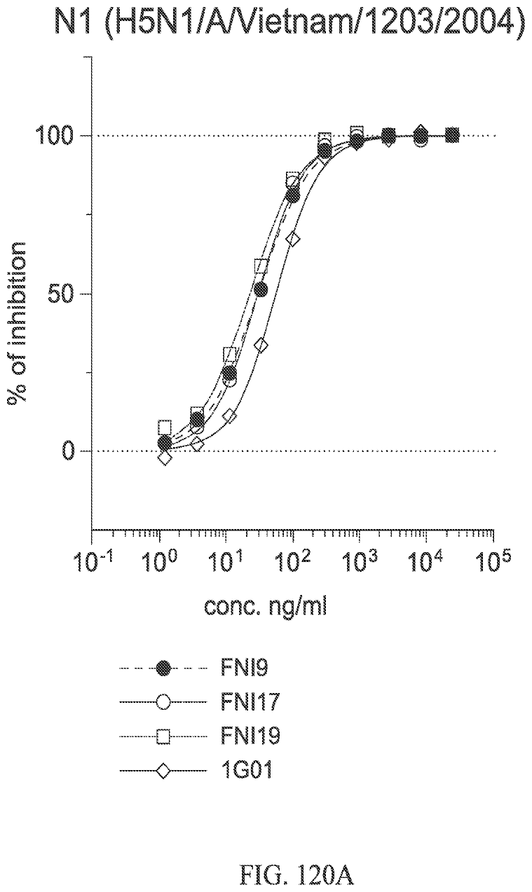

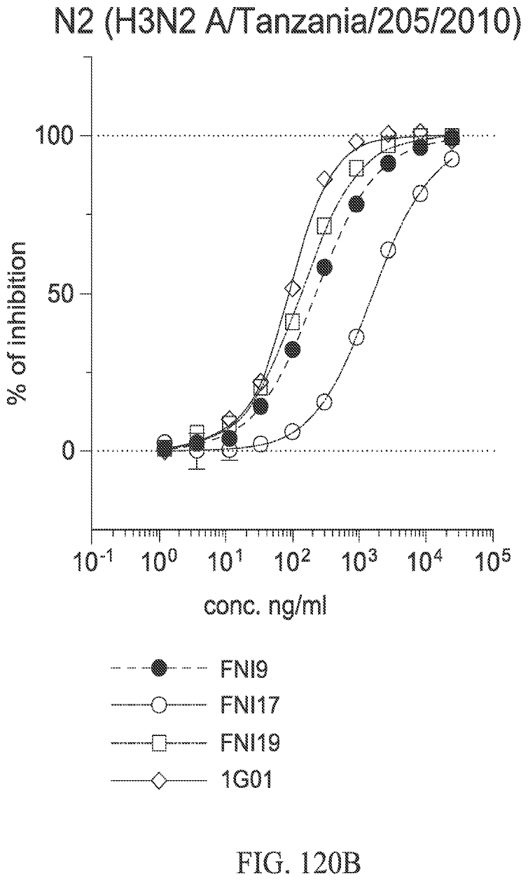

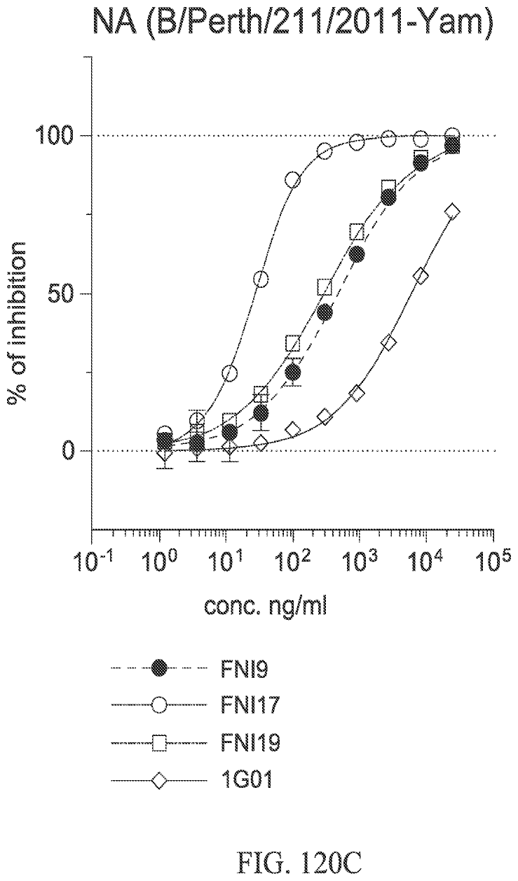

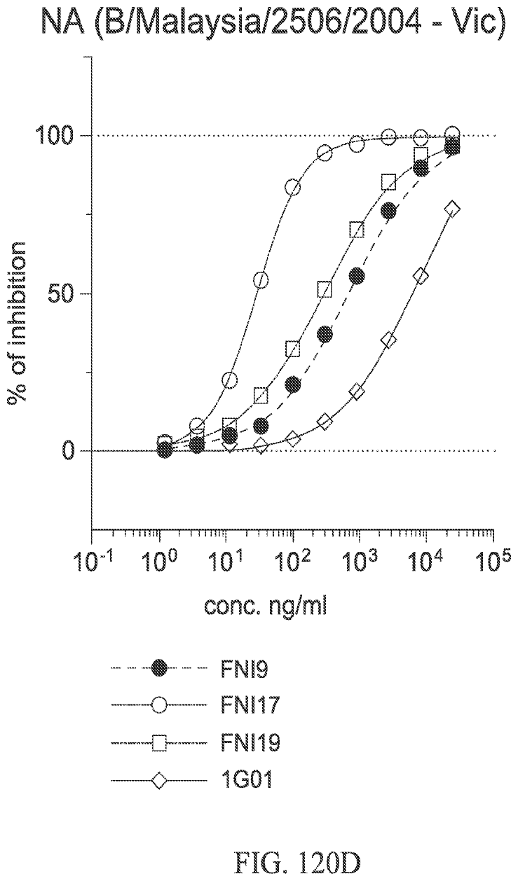

Antibody-dependent cell cytotoxicity (ADCC) mediated by FNI9-v8.1-LS on A549 cells infected with H1N1 A/Puerto Rico/8/34 in the presence of human natural killer cells was measured (FIGS. 115A-115B). ADCC of an anti-HA comparator antibody, FM08_LS, and a Fc-silent negative control, FNI9-v8.1-GRLR, was also measured. ADCC is reported as % antibody-dependent killing in FIG. 115A and area-under-the-curve in FIG. 115B.

### Example 25

### Pharmacokinetic Studies: FNI9-v5 and FNI9-v8.1

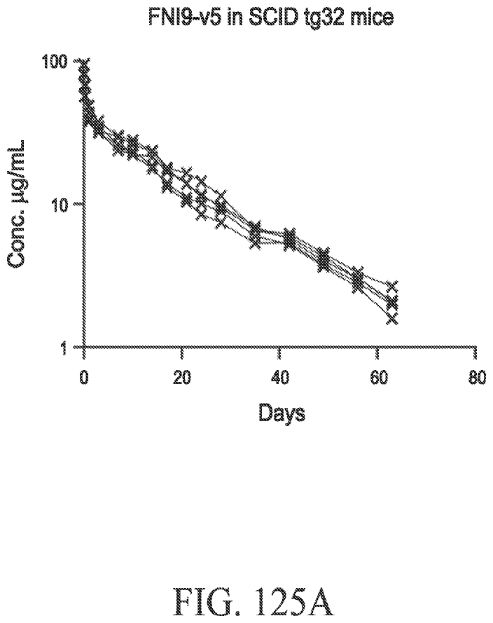

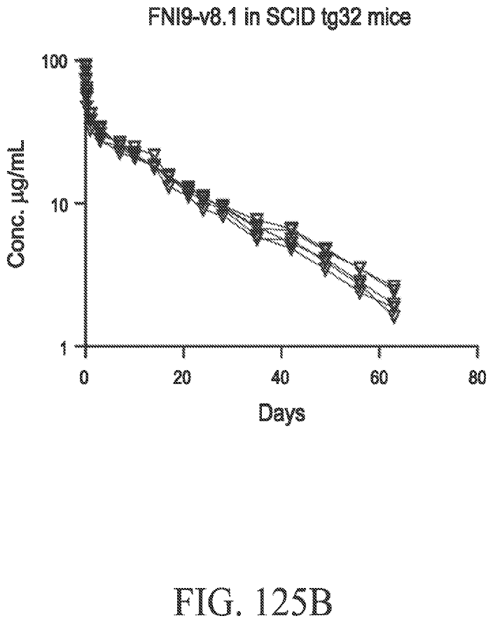

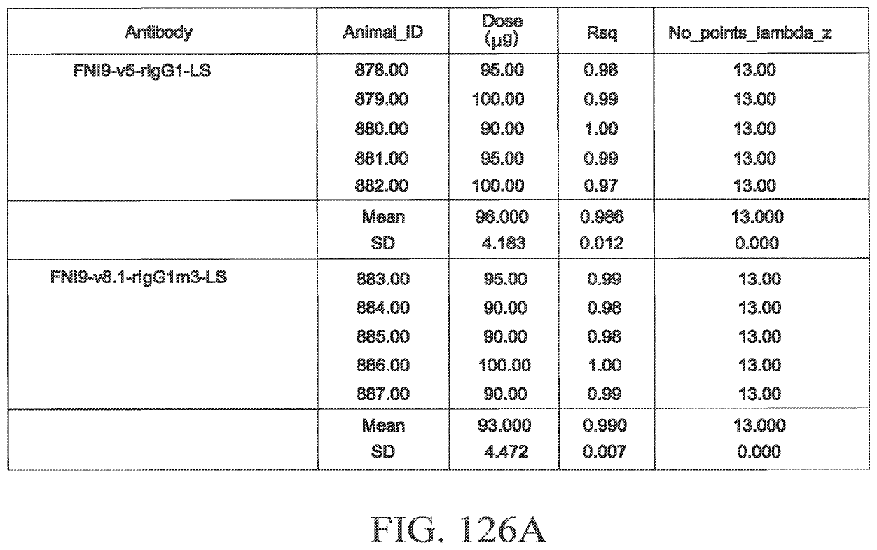

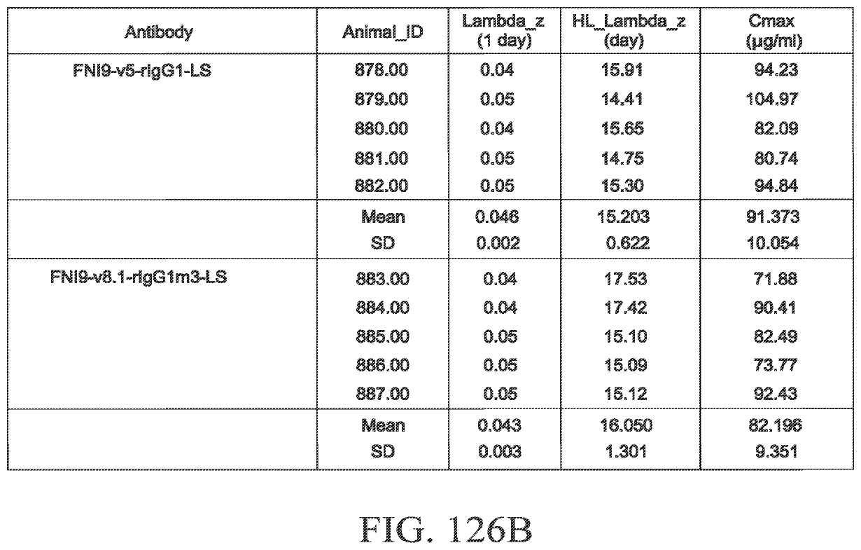

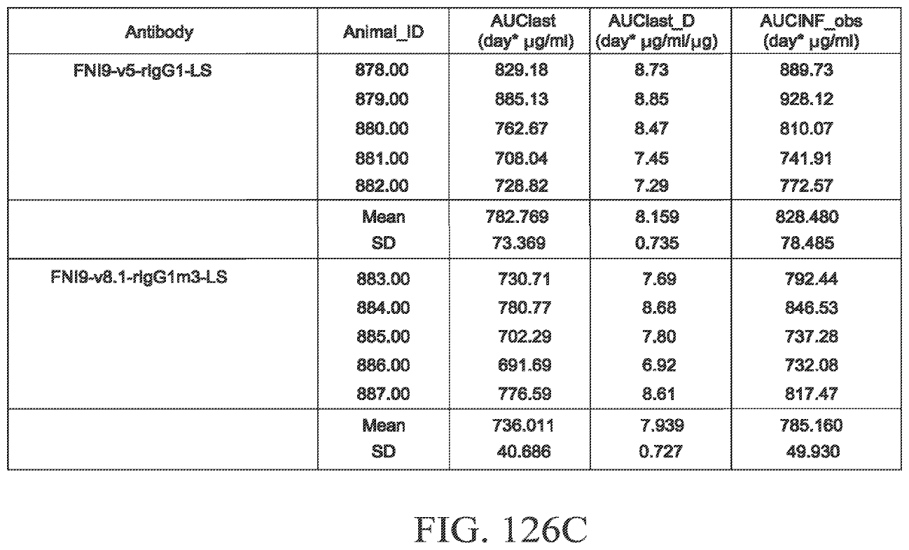

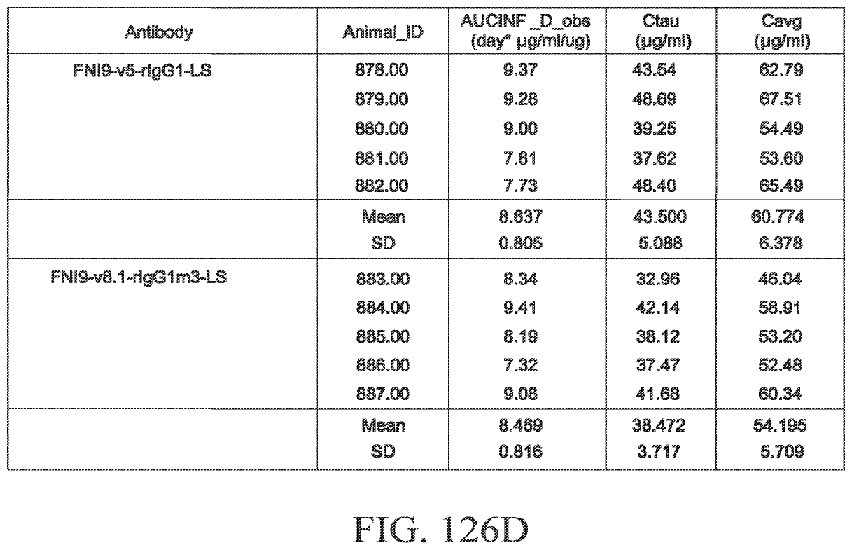

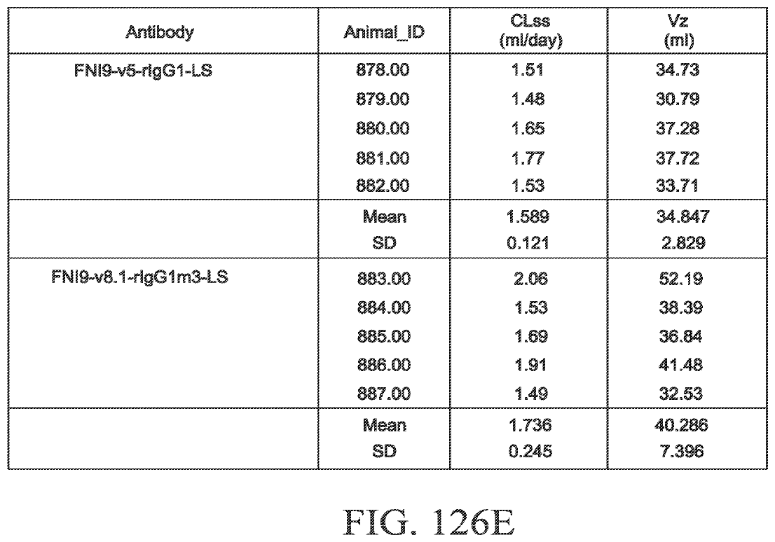

In vivo pharmacokinetics of FNI9-v5 and FNI9-v8.1 were tested in SCID tg32 mice. Antibodies were expressed as recombinant IgG1m3 with M428L and N434S mutations in the Fc. Antibody concentration over time (reported as μg/ml) for FNI9-v5 and FNI9-v8.1 in SCID tg32 mice over approximately 60 days post-administration is shown in FIGS. 125A and 125B, respectively. FIGS. 126A-126E summarize in vivo pharmacokinetic data of FNI9-v5-LS (“FNI9-v5-rIgG1-LS”) and FNI9-v8.1-LS (“FNI9-v8.1-rIgG1m3-LS”) in SCID tg32 mice (n=five individual animals per antibody).

### Example 26

### Tissue Cross-Reactivity (TCR) Assessment: FNI9-V5 and FNI9-V8.1

The potential for tissue cross-reactivity (TCR) of FNI9-v5 and FNI9-v8.1 antibodies was assessed in a panel of human tissues using immunohistochemistry staining.

To facilitate immunohistochemical detection, FNI9-v5, FNI9-v8.1 (each expressed as recombinant IgG1m3 with M428L and N434S mutations in the Fc), and an isotype control (“ctrl-IgG1”, targeting a non-influenza antigen) were conjugated with Alexa Fluor 488 using the commercial Alexa Fluor™ 488 protein labelling kit. The presence of the conjugated protein was determined by measuring the absorbance at 280 nm. The respective conjugated antibody protein concentrations were 2.17 mg/mL (FNI9-v5), 1.49 mg/mL (FNI9-v8.1), and 1.62 mg/mL (ctrl-IgG1). The respective degrees of labelling were 1.63 (FNI9-v5), 0.86 (FNI9-v8.1), and 2.56 (ctrl-IgG1).

Cryo-sections from influenza-positive and influenza-negative cells were prepared as controls and a panel of human tissues for examination were prepared. The panel included adrenal, urinary bladder, blood cell, bone marrow, breast, brain-cerebellum, brain-cortex, endothelium, eye, fallopian tube, oesophagus, gastric antrum, gastric body, duodenum, ileum, colon, heart, kidney, liver, lung, lymph node, ovary, pancreas, parotid, peripheral nerve, pituitary, placenta, prostate, skin, spinal cord, spleen, striated muscle, testis, thymus, thyroid, tonsil, ureter, uterine-cervix), and uterine-endometrium tissues. To assess tissue viability, the panel of human tissues was stained immunohistochemically to demonstrate vimentin, cytokeratin and von Willebrand factor. The assessment of tissue viability indicated that the panel of human tissues was viable.

FNI9-v5-AF488 was titrated in control samples (influenza positive and negative cells). Positive membranous and cytoplasmic concentration-dependent staining of individual cells was observed throughout the preparation of influenza positive cells (positive control). No FNI9-v5-AF488 positive staining was observed in influenza negative cells (negative control). No staining was seen in either control sample with ctrl-IgG1-AF488 or the antibody diluent.

Following slide evaluation of the positive control titration, three concentrations were selected for testing in the human tissue panel: 1.25, 0.16, and 0.020 μg/mL. To confirm suitability of the staining method and concentrations used for FNI9-v5-AF488 for use with FNI9-v8.1-AF488, a method transfer was completed where the control samples were stained with FNI9-v8.1-AF488 at the three previously used concentrations: 1.25, 0.16 and 0.020 μg/mL. Staining observed in the positive control sample with FNI9-v8.1-AF488 was consistent with the staining previously observed with FNI9-v5-AF488 and the staining method and concentrations were therefore considered suitable for use in the tissue titration using FNI9-v8.1-AF488.

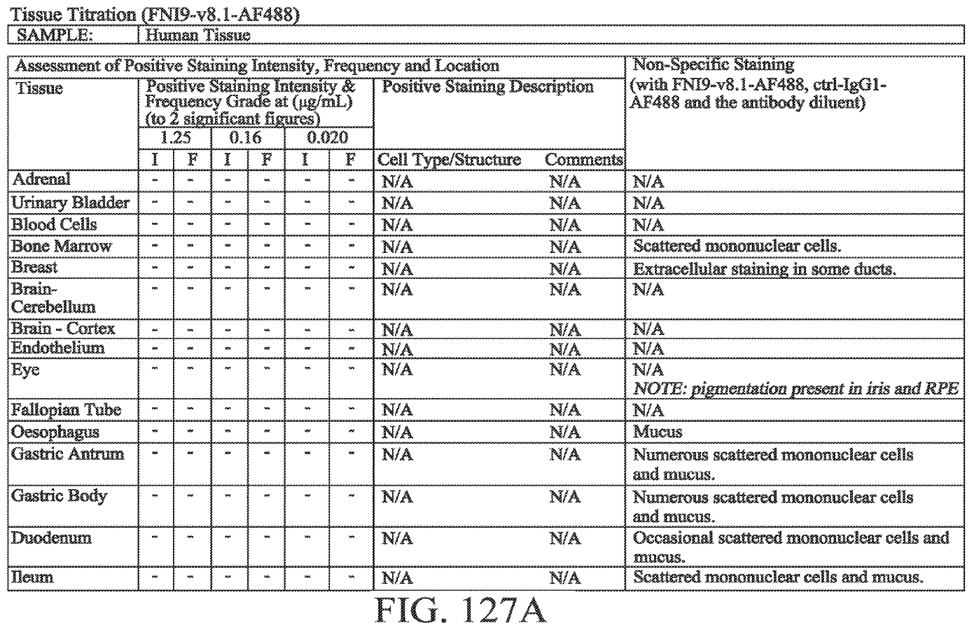

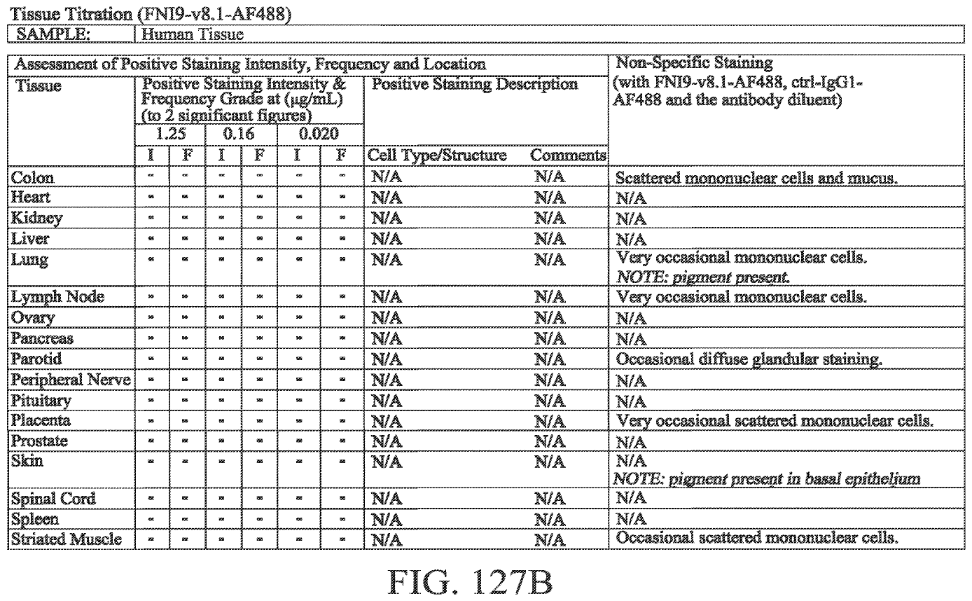

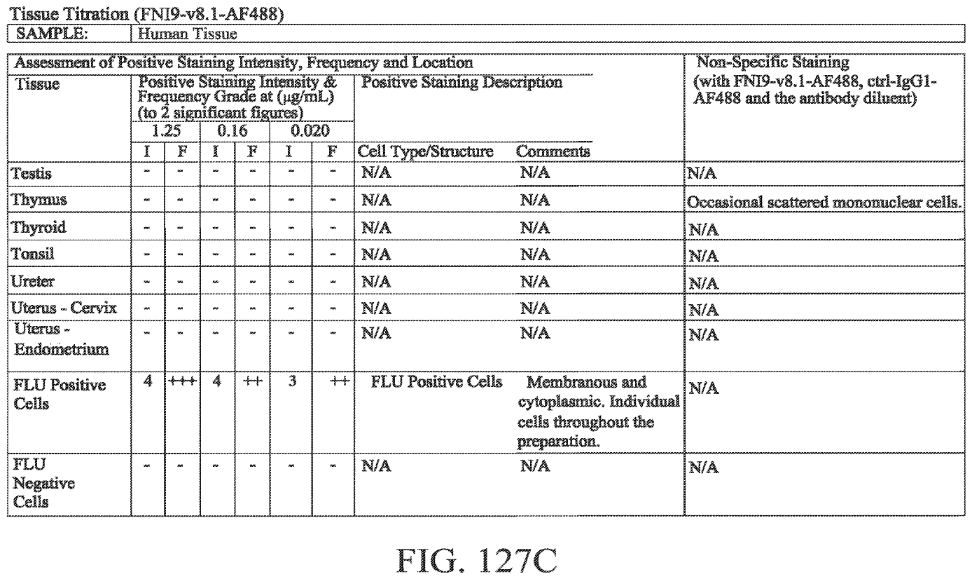

Tissue titration was performed in the panel of human tissues. Positive staining with FNI9-v5-AF488, consistent with that observed in the control titration, was observed in the positive control sample and comparable staining was observed with FNI9-v8.1-AF488. No positive staining was observed with FNI9-v5-AF488 or FNI9-v8.1-AF488 in the negative control sample. No positive staining was observed with FNI9-v5-AF488 (data not shown) or FNI9-v8.1-AF488 (FIGS. 127A-127C) in any of the human tissues.

### Example 27

### Testing of FNI Antibodies Against Low Pathogenic IAVs

Inhibition of neuraminidase in low pathogenic IAVs by FNI antibodies was evaluated using in vitro assays.

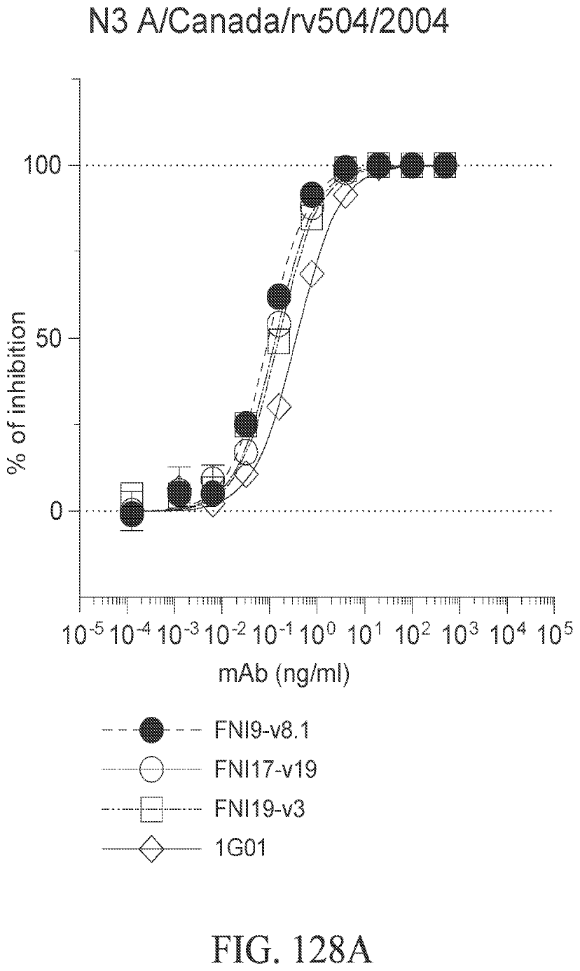

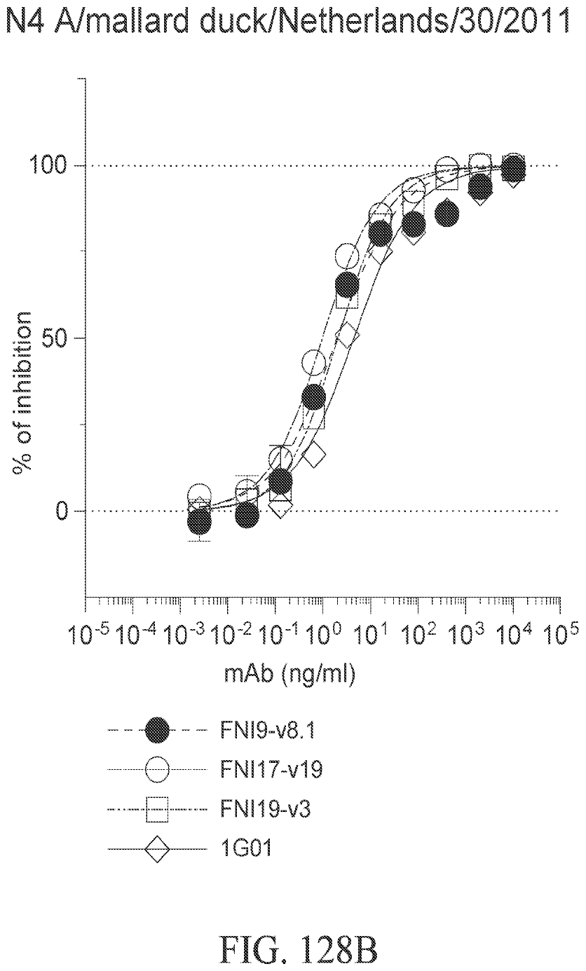

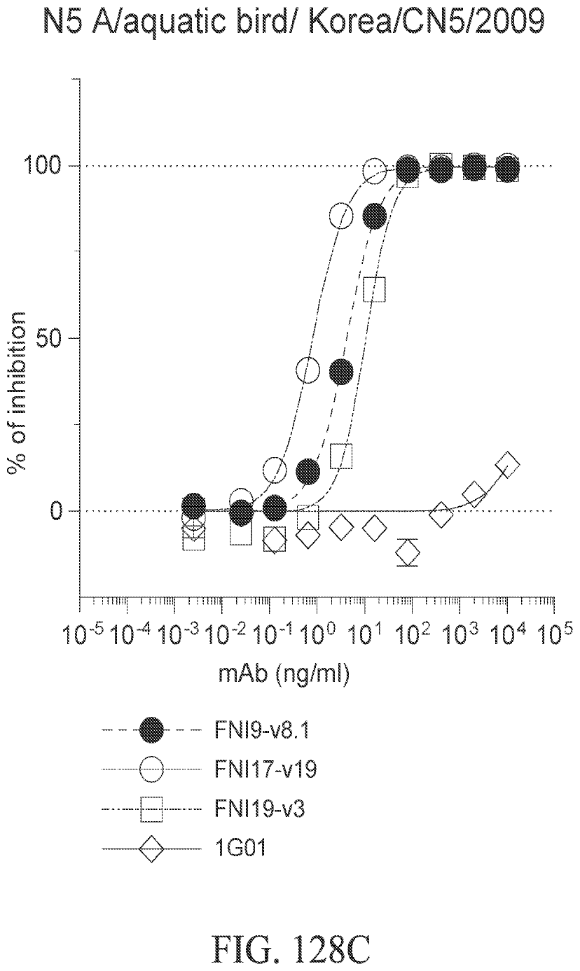

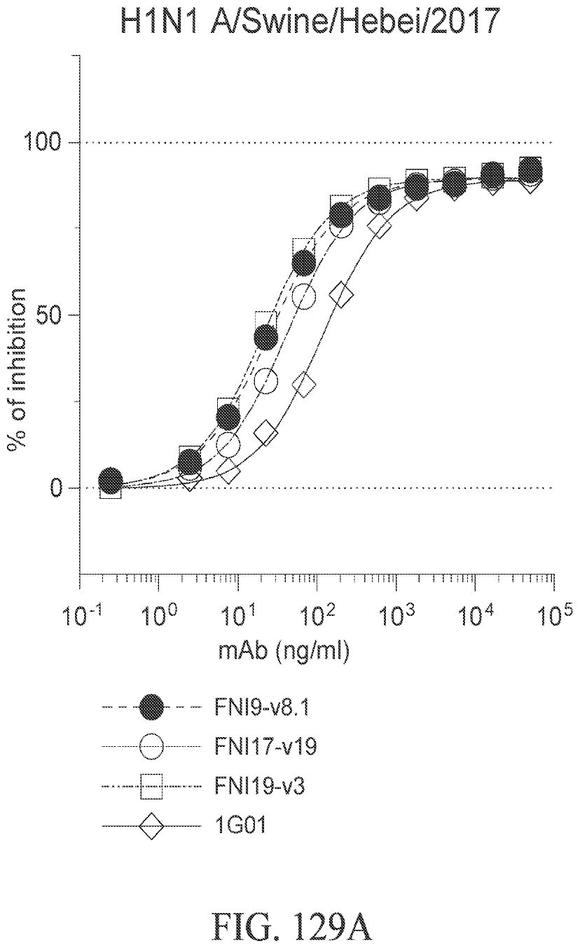

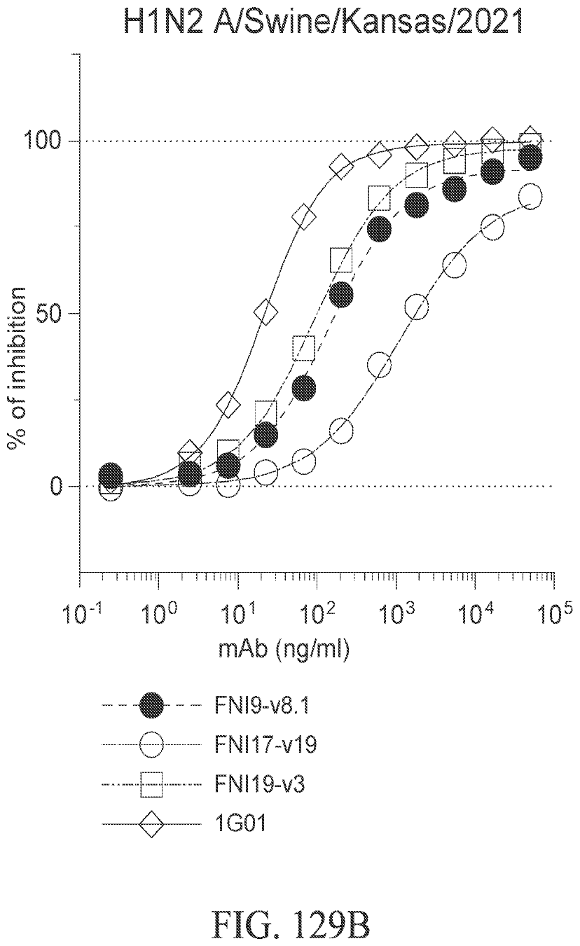

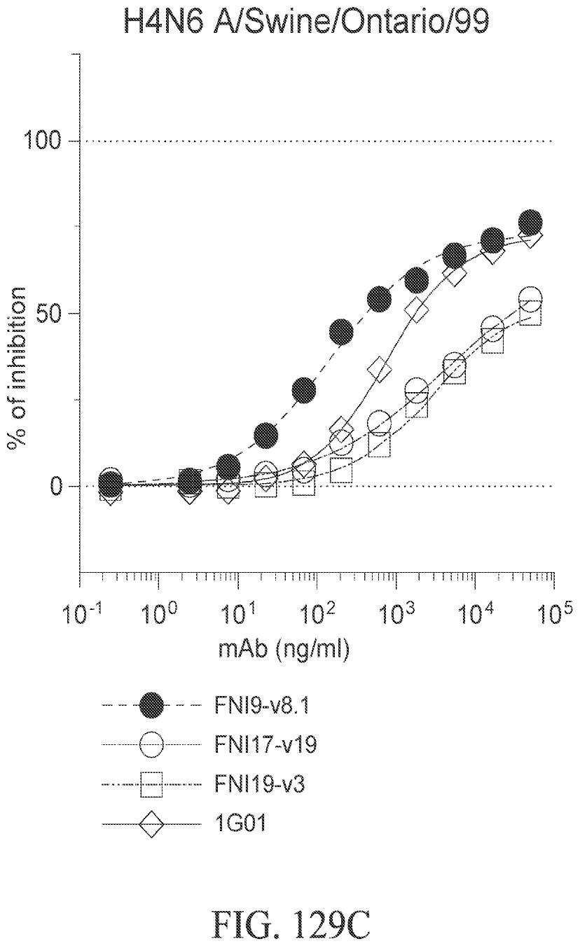

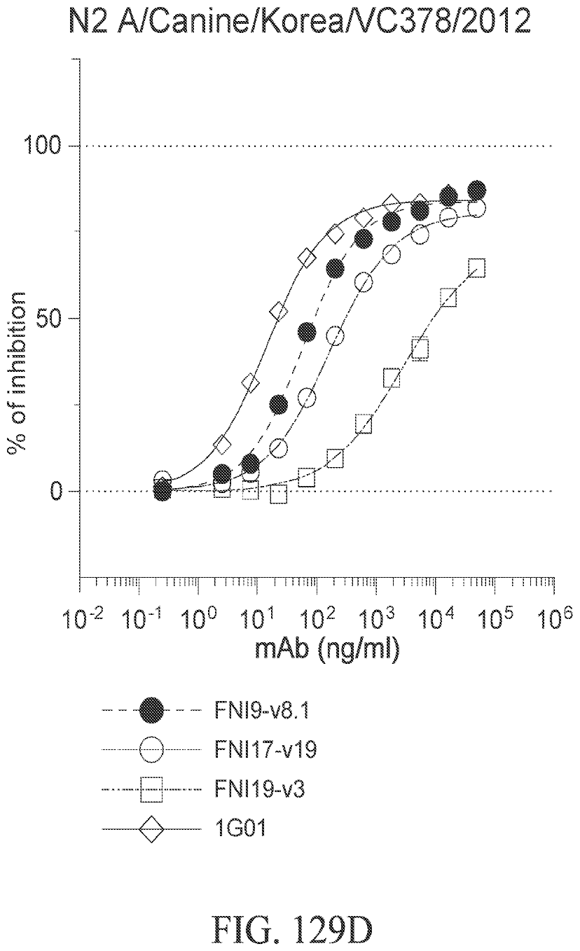

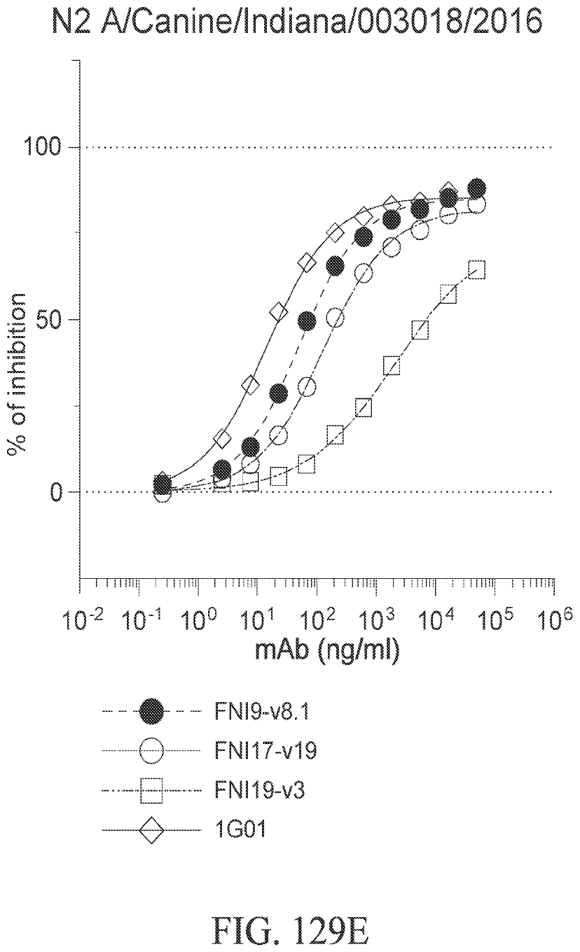

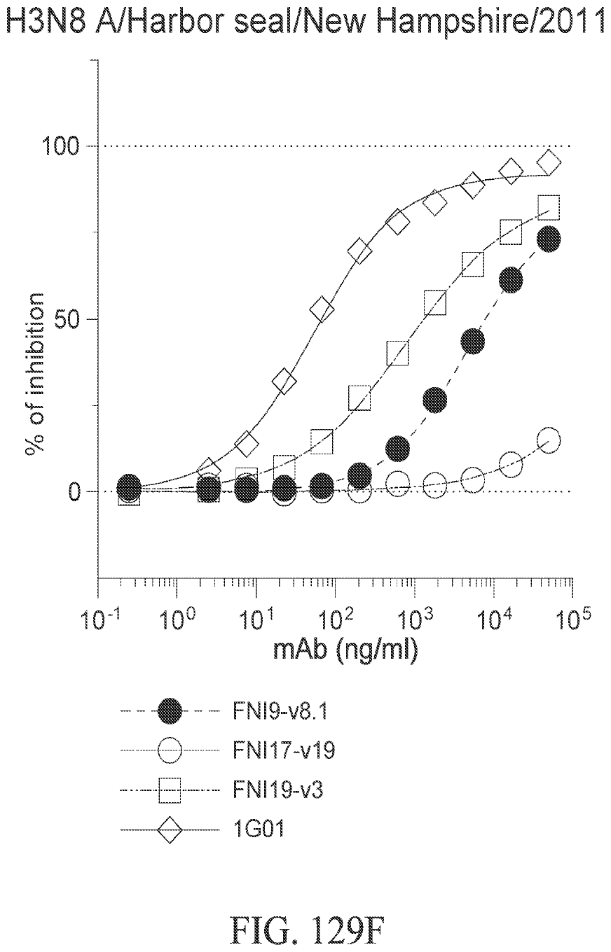

Inhibitory activity of anti-NA mAbs FNI9-v8.1, FNI17-v19, and FNI19-v3 against NA-only based pseudotypes bearing N3, N4, or N5 representative of enzootic low pathogenic avian influenza A viruses (LPAIVs) was evaluated by ELLA (FIGS. 128A-128C). Inhibitory activity of the same anti-NA mAbs against NA-only based pseudotypes bearing N1, N2, N6, or N8 representative of enzootic low pathogenic mammalian IAVs circulating in swine, dogs, and seals was evaluated by MUNANA assay (FIGS. 129A-129F). 1G01 was also tested as a control antibody in both the ELLA and MUNANA assay.

### Example 28

### Testing of FNI Antibodies Against Resistant IAV Mutants

### In Vitro Resistance Studies

Antibody resistant mutants were isolated by screening the viral population in bulk or by serially passaging the virus in the presence of an escalating dose of antibody. For the former approach, MDCK cells were plated into 96-well plates (Corning CON3596) at 30,000 cells/well in 100 μl of culture medium (MEM+GlutaMAX (ThermoFisher 41090-028)+10% FBS Hyclone (VWR SH30070.03)+50 U/ml Penicillin/50 ng/ml Streptomycin (Bioconcept 4-01F00-H)+100 μg/ml Kanamycin (Life Technologies 15160047)+1% NEAA (Bioconcept 5-13K00-H)+1% Sodium Pyruvate (Life Technologies 11360039)+0.05 mM β-mercaptoethanol (Bioconcept 5-69F00-E)) and cultured over-night at 37° C. 5% CO2. The day after, A/Hong Kong/1/1968 virus input was pre-incubated with 100 μg/ml of FNI19 antibody for 30 min at 37° C. The virus/antibody mix was then limiting diluted in infection medium (MEM supplemented with 1 μg/ml TCPK-trypsin (Bioconcept LS003750) and 10 μg/ml kanamycin) to infect MDCK cells at MOI=0.001 in 100 μl, after 2 washing steps with 200 μl/well of PBS (Sigma-Aldrich D8537-500ML). Three hours later, 100 μl/well of infection medium containing 100 μg/ml FNI19 was added, and cells incubated for further 72 hours. After the incubation time, a 20 mM MUNANA substrate (Sigma 69587-5MG) solution was prepared in MUNANA buffer (MES 32.5 mM/CaCl₂) 4 mM, pH 6.5) and 50 μl/well dispensed into 96-well plate to be incubated with an equal amount of assay culture supernatant for 1 hour at 37° C. Hundred μl/well of stop solution (0.2 M glycine/50% Et-OH, pH 10.7) was added and fluorescence at 445 nm measured with a Cytation 5 (Biotek-Agilent). The fluorescence threshold was set to 5× the signal of not infected cells and supernatants above that cut off were processed for NA sequencing. Viral genomic RNA was extracted from 140 μl of culture supernatant using Qiagen QIAmp Viral RNA kit (Qiagen 52904) according to manufacturer's instruction. cDNA was synthetized using SuperScript III RT kit (Life Technologies 18080044) with the primer Uni12 (5′-AGC RAA AGC AGG-3′ (SEQ ID NO:113)). The neuraminidase gene was amplified by PCR using the Q5 HotStart DNA polymerase kit (Bioconcept M0493L) with a specific primer forward: 5′-caattggctctgtctctct-3′ (SEQ ID NO.: 114) and reverse: 5′-atgaaattgatgttcgccc-3′ (SEQ ID NO:115).

PCR product was purified from 1.5% agarose gel and sequenced with the 3 different primers: 5′-caattggctctgtctctct-3′ (SEQ ID NO:114), 5′-gaagagccgatactagaa-3′ (SEQ ID NO:116), 5′-ttctagtatcggctcttc-3′ (SEQ ID NO:117). Sequence alignment with A/Hong Kong/1/1968 neuraminidase was performed with CLC Main Workbench 22 software.

For the serial passaging resistance experiment, MDCK-LN cells were seeded in 24-well plates at 120,000 cells/well and cultured overnight at 37° C. in growth media (DMEM, 10% [FBS], 0.01M HEPES, 100 U/mL Penicillin-100 μg/mL Streptomycin). Twenty-four hours later, H1N1 A/California/07/2009 or A/New Caledonia/20/99 virus stock and FNI9 were diluted and mixed 1:1 in infection media containing 0.5 μg/mL TPCK-treated Trypsin for a final concentration of 1000 PFU/well virus and 0.5×IC50 of FNI9 (47.35 ng/mL). The virus:antibody mixture were placed at 37° C. and precomplexed for 30 minutes. Cell and virus control wells were included for CPE comparators. The cells were washed twice in DMEM and then 100 μL of the virus:antibody complexed mixture, virus, and cell controls were added to the wells for 1 hour at 37° C. After virus adsorption, the cells we washed twice in DMEM then 500 μL of diluted antibody or infection media containing 0.5 μg/mL TPCK-treated Trypsin were added back to their respective wells. The plates were incubated at 37° C. and the virus was collected once there was over 50% CPE in the virus:antibody well. The virus supernatant was frozen in 120 μL aliquots at −80° C., one aliquot used for the next resistance passage, one aliquot was added to 360 μL of DNA/RNA Shield for RNA extraction, and one aliquot was saved for additional propagations. For passage 2, the process was repeated with the original 0.5× and a 1×IC50 concentration of FNI9, with the highest concentration of antibody showing CPE being selected for the next passage. For each additional passage, the concentration from the selected well was repeated along with a 2× concentration. For the CA09 study, there were 8 passages of virus and a final concentration of 64× the IC50 of FNI9 (3200 ng/mL).

For identification of genomic mutations, viral genomic RNA was extracted using the Zymo QuickRNA Viral Kit (R1034) according to the manufacturer's instructions and the RNA concentration was measured using the Qubit RNA BR Assay Kit (Invitrogen, Q10210). cDNA was synthesized using SuperScript III One-Step RT-PCR system with Platinum Taq DNA polymerase (Invitrogen, 12574018) using the MBTuni-12 (5′-ACGCGTGATCAGCAAAAGCAGG-3′ (SEQ ID NO:118)) and MBTuni-13 (5′-ACGCGTGATCAGTAGAAACAAGG-3′ (SEQ ID NO:119)) primers, and the PCR fragments were purified using AMPure XP magnetic beads (Beckman Coulter, A63880). Next, the amplicons were fragmented using NEBNext Ultra II FS DNA Library Prep Kit for Illumina (NEB, E6177L), then adaptors and indexes were added using NEBNext Multiplex Oligos for Illumina (NEB, 7335L). DNA samples were purified using NEBNext Sample purification beads, and then quantified using the Qubit DNA HS Assay kit (Invitrogen, Q32854), and verified using a D500 tape station (Agilent, G2964AA). Finally, the samples were diluted and pooled, then sequenced using a MiSEQ analyzer with MiSeq Reagent Kit v3 (MS-102-3003) and PhiX Control v3 (FC-110-3001). The NA mutations 1223R and S247N first appeared in passage 6 and 7 respectively, and each reached about 50% of the total NGS reads by passage 8.

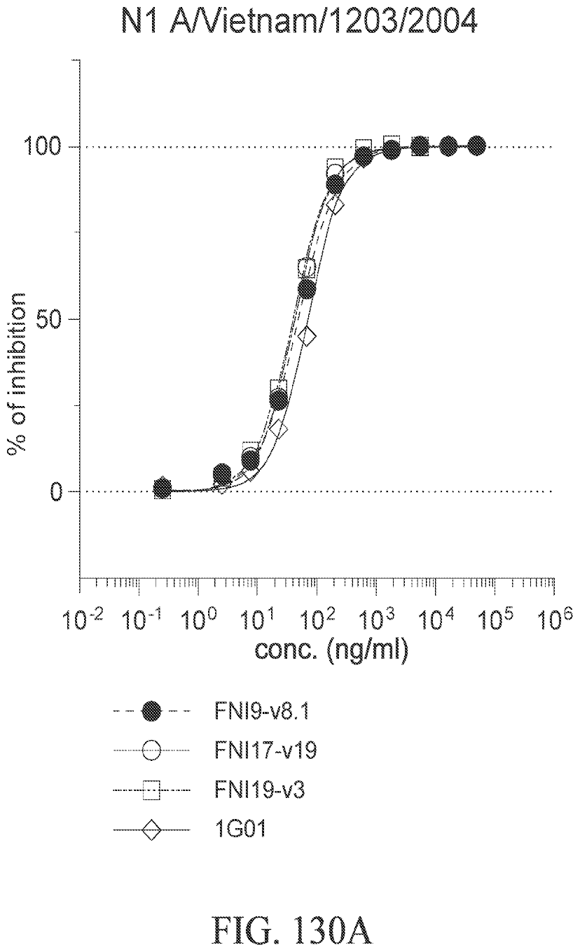

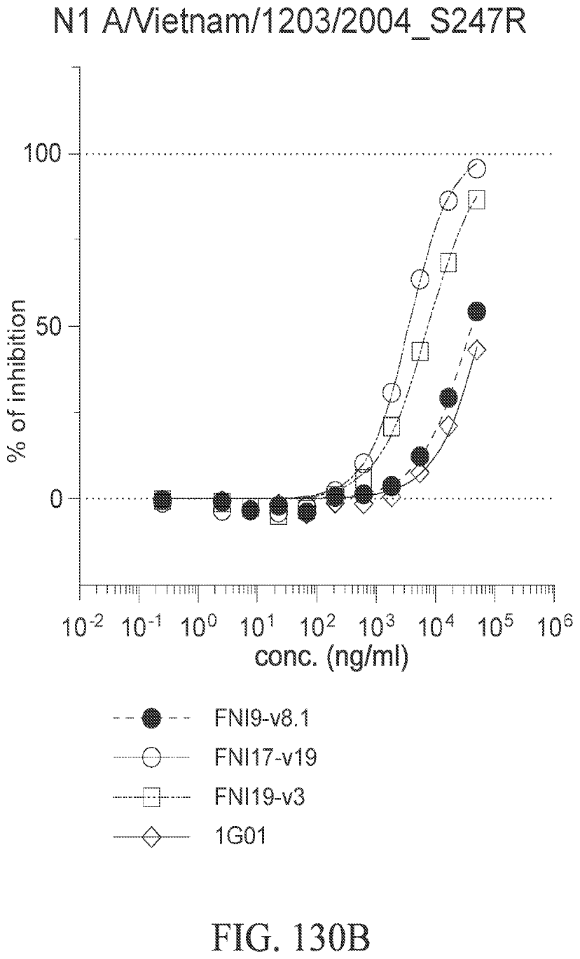

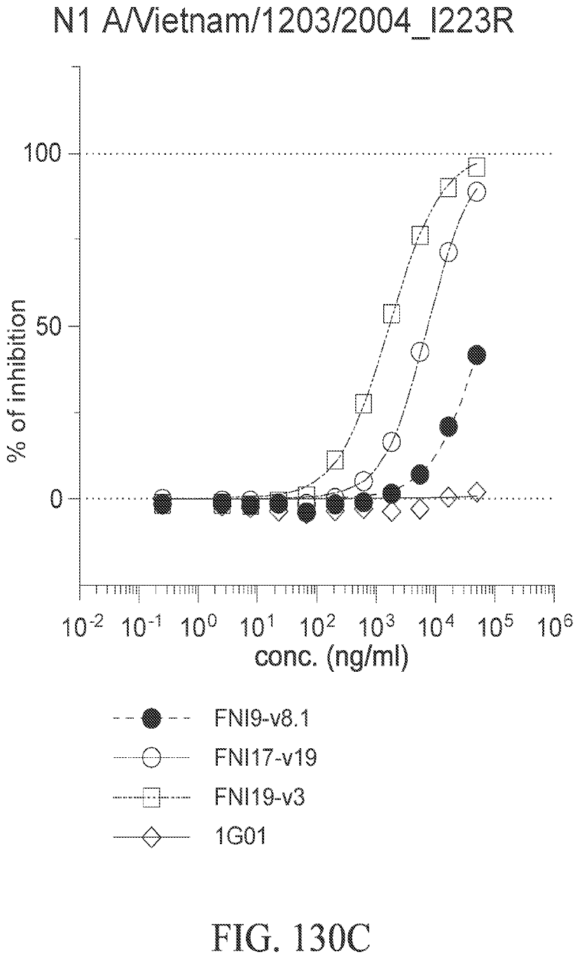

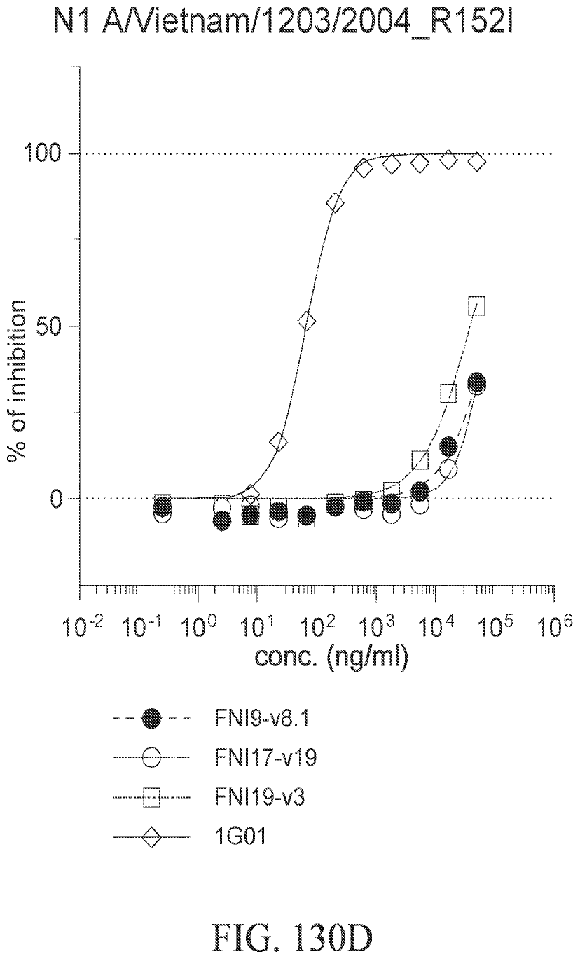

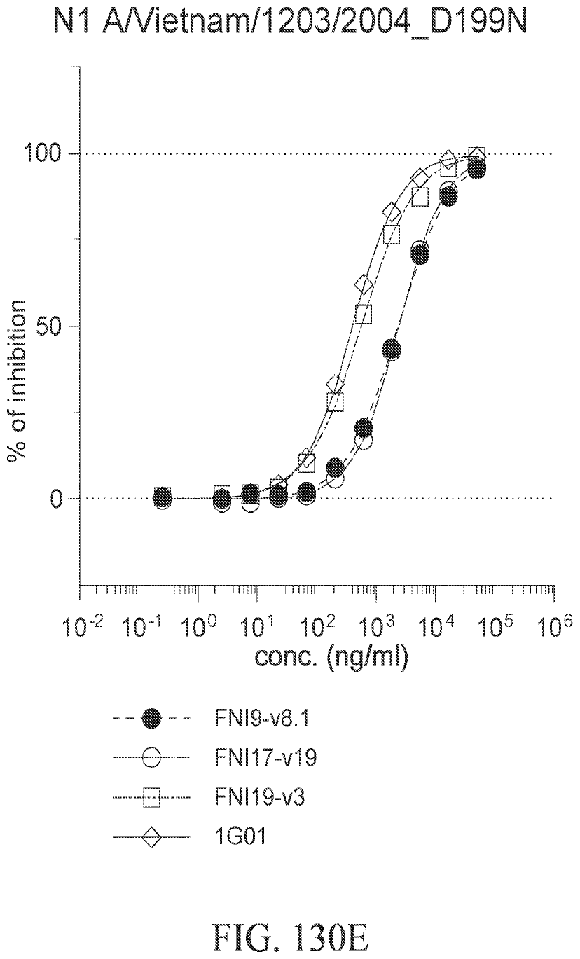

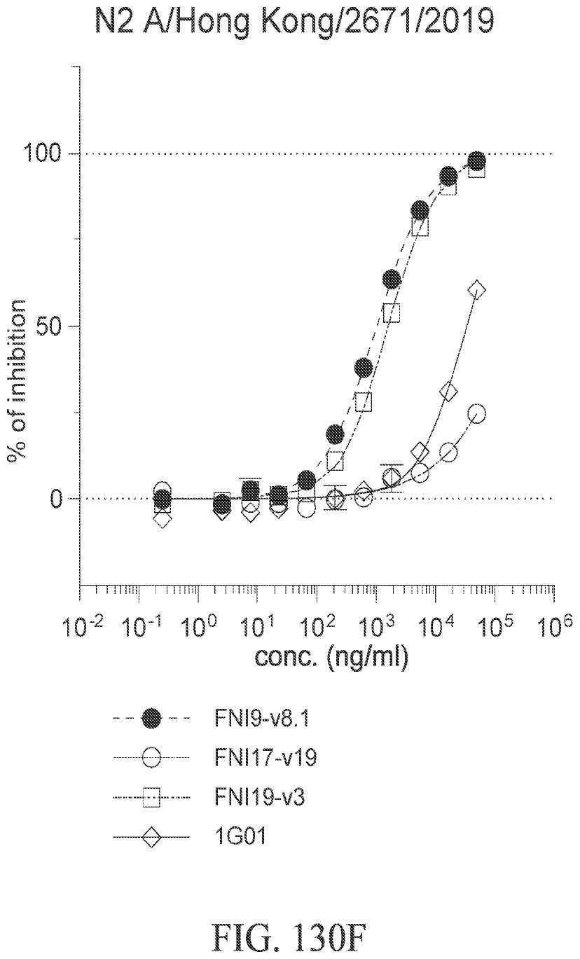

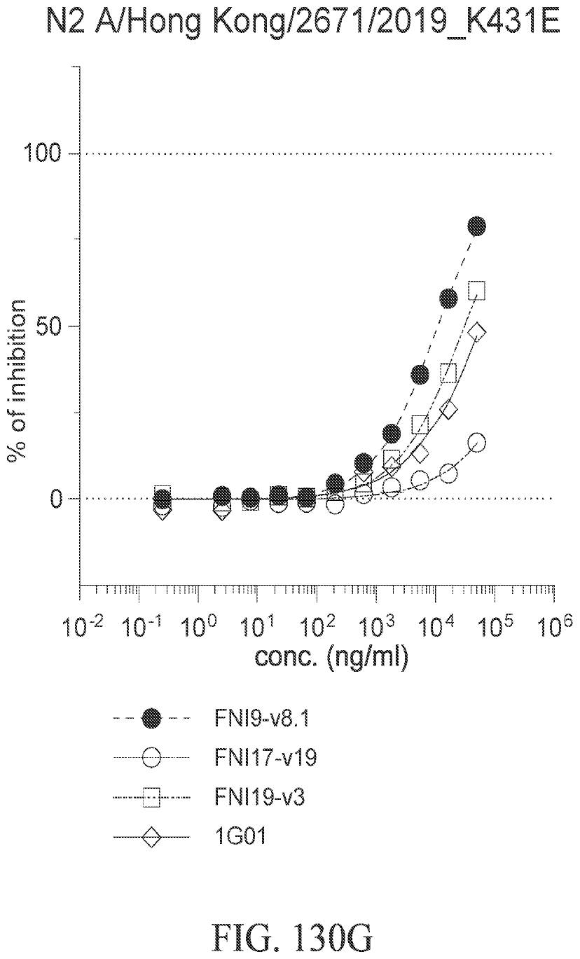

A panel of NAs with mutations identified through in vitro resistance studies as capable of reducing, but not abrogating, the binding or activity of FNI mAbs was identified. Inhibition of neuraminidase enzymatic activity against the panel was measured by MUNANA assay using pseudotypes bearing the mutated NAs (FIGS. 130A-130G).

### Deep Mutational Scanning Profiles

A deep mutational scanning approach was used to determine how NA amino acid mutations affect mAb binding. A cell-surface display library based on N1 A/California/07/2009 was generated with all variants for 170 select amino acids included. The mutated residues include epitope residues within 6.0 A distance from bound Fabs obtained from cryo-EM (FNI17 Fab and FNI19 Fab in complex with N2/A/Tanzania/205/2010, this study) and crystal structures (N13 Fab with N2/A/Tanzania/205/2010 (unpublished), 1E01 Fab (PDB 6Q20), Mem5 Fab (PDB 2AEP) and B10 Fab (PDB 6N6B). In addition, residues with known sequence variation in GISAID in the past 20 years for H1N1 and H5N1 were included.

Library construction, lentivirus production, next-generation sequencing and data deconvolution was performed by the Genetic Perturbation Platform (GPP) of the Broad Institute of MIT and Harvard. For library synthesis, the lentiviral vector pMT025 encoding the wildtype N1 A/California/07/2009 ORF with addition of a C-terminal minimal HA tag via a 2×G4S linker was synthesized and used as a template to generate the variant library pool. The full-length variant pool with 5′ and 3′ flanking adapters containing NheI and MluI respectively, was synthesized at Twist Biosciences. Following restriction digest cloning and pDNA amplification, the variant composition of the pDNA library was assessed by NGS using the Illumina Nextera XT platform. All 3472 designed variants were detected and the distribution of the variants was approximately log-normal with a 1.3-fold standard deviation in the read counts across the variants. Lentiviral particles were produced according to protocols available through the GPP (http://www.broadinstitute.org/mai/public/resources/protocols). The library was transduced into FreeStyle™ 293-F Cells (ThermoFisher Scientific) at a multiplicity of infection of ˜0.3 and maintained at an average representation of 2,000 cells per variant.

For the screen, mAb binding to cell-surface displayed NA was determined by flow cytometry. mAbs were directly conjugated to Alexa Fluor 647 (ThermoFisher Scientific) through free amine conjugation to enable fluorescence-based detection. 293-F cells containing the NA variant library were harvested 5-7 days after puromycin selection, washed in PBS containing 3% bovine serum albumin then incubated with Alexa Fluor 647-conjugated mAbs for 1 h at 4° C. in the dark. Cells were washed then fixed with 4% paraformaldehyde, then sorted in technical duplicate on two separate sorters (BD Aria Fusion or Sony MA900). Dead cells were excluded from the analysis using the LIVE/DEAD™ Fixable Blue Dead Cell Stain Kit (ThermoFisher Scientific). A minimum of 60 million cells was prepared for FACS per technical replicate. 293-F cells expressing the NA variant library were sorted by FACS into negative, low and high mAb binding bins and the distribution of variants in each bin was determined by NGS as described below. The NA ORF includes a minimal c-terminal HA tag which was also screened against using the Alexa Fluor 488 anti-HA.11 Epitope Tag antibody (BioLegend) to determine how mutations at each of the 170 selected residues affect general protein expression. For each screen a minimum of 5 million unsorted library cells were collected to serve as unsorted controls.

gDNA was isolated from sorted samples and unsorted controls with the QIAamp DNA FFPE Tissue Kit (with the following modification: 0.3 M NaCl was added to the lysis buffer and incubation at 56° C. extended to 4 h) and the QIAamp DNA Blood Kit (Qiagen) respectively. gDNA was prepared for NGS as previously described112. Briefly, the ORF is amplified from gDNA by PCR with the following primers (Forward: 5′-ATTCTCCTTGGAATTTGCCCTT-3′ (SEQ ID NO:120), Reverse: 5′-CATAGCGTAAAAGGAGCAACA-3′ (SEQ ID NO:121)) and processed for NGS using the Illumina Nextera XT kit. Samples were sequenced using the Nextseq2000 sequencing platform to obtain paired-end reads with 2×150 cycles. Sequence data processing and variant calling using the mutant detection software ASMv1.0 was performed as previously described112. Normalized reads for each sample are log 2 transformed. Log2 fold change values comparing the negative sorted bin to the unsorted pool and the low and high binding FACS sorted bins for each library variant was computed and then averaged both for the anti-HA-tag and the anti-NA antibody. Potential escapes were defined as those having an HA-tag signal close to the silent mutations (max two standard deviations away from the mean of the silent mutations) and having an anti-NA signal close to the stop codon mutations (max three standard deviations away from the mean of the stop codon mutations).

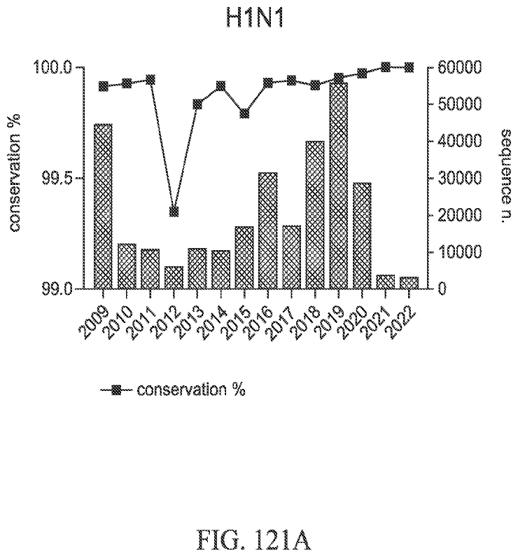

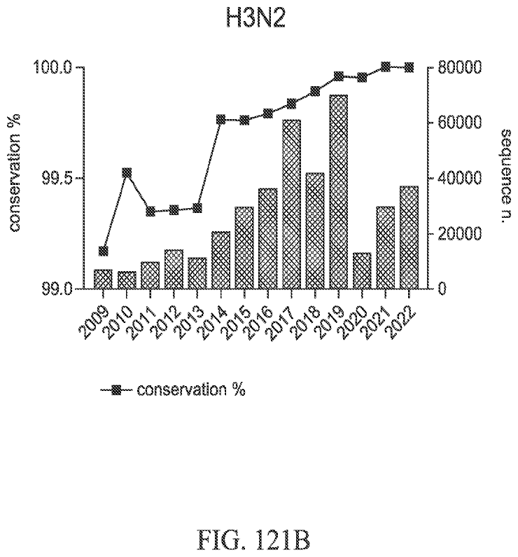

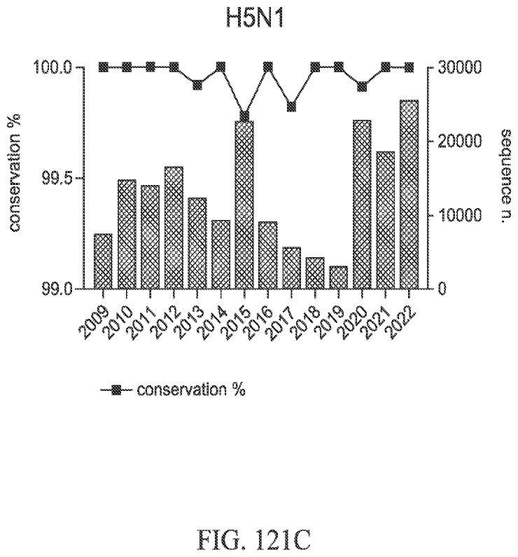

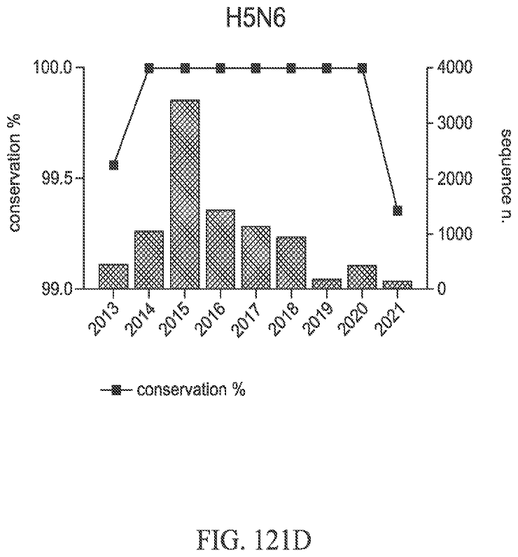

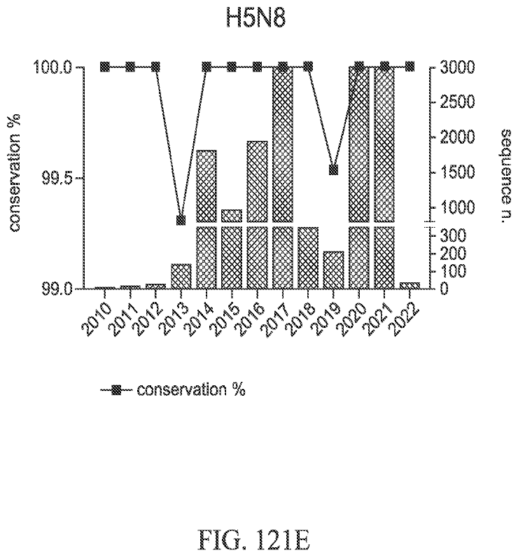

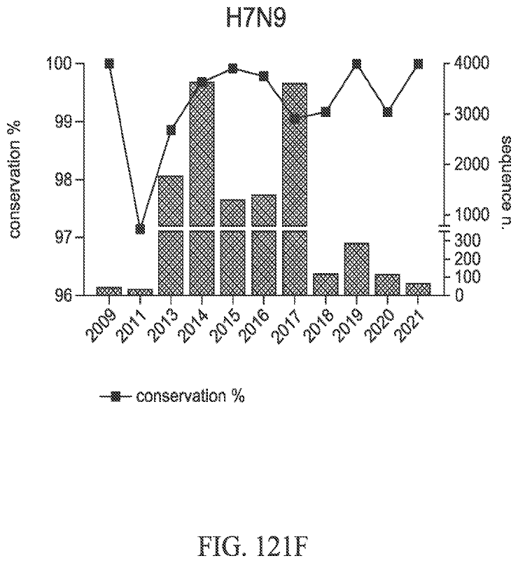

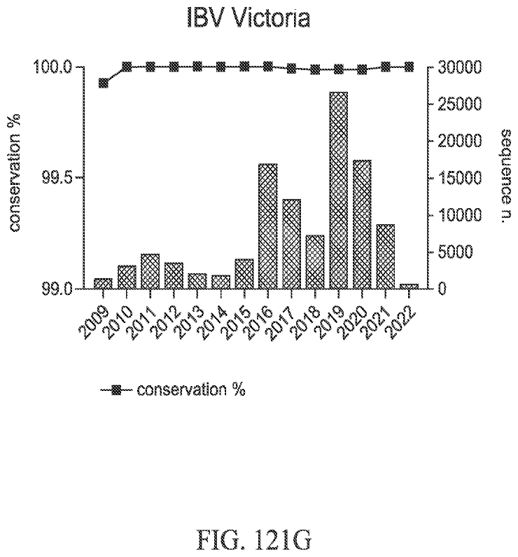

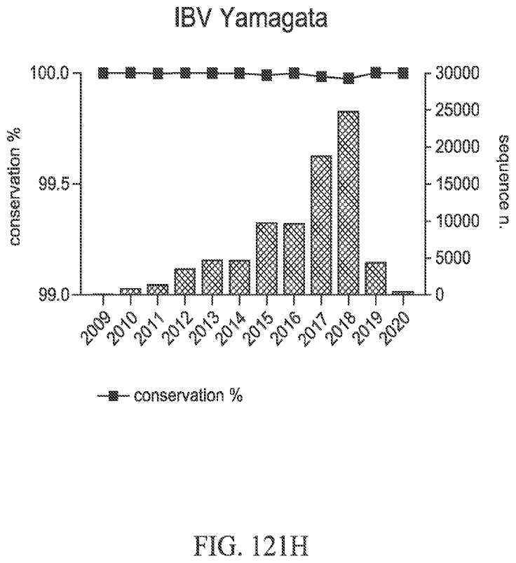

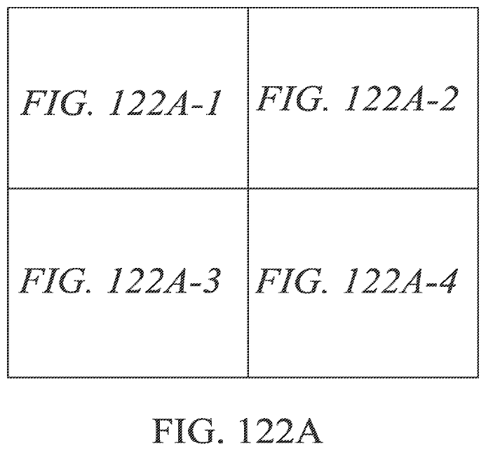

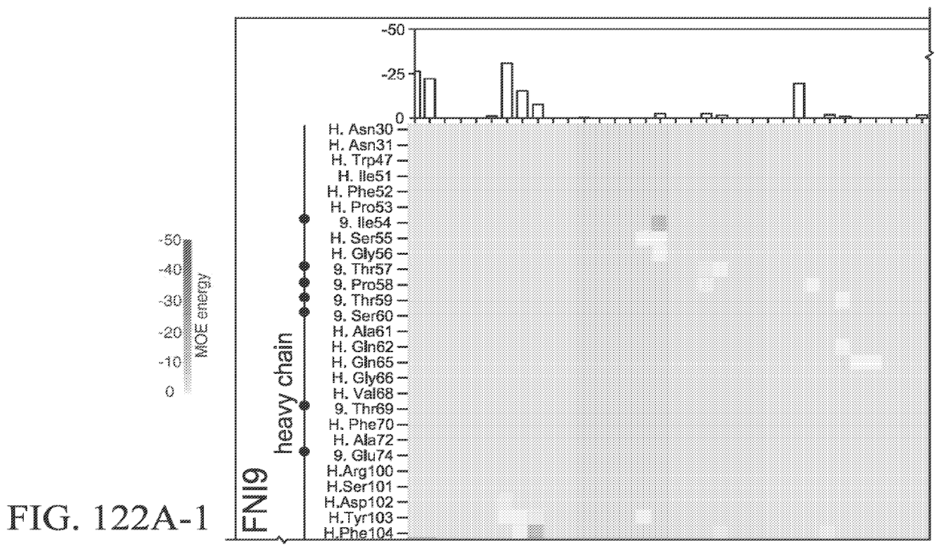

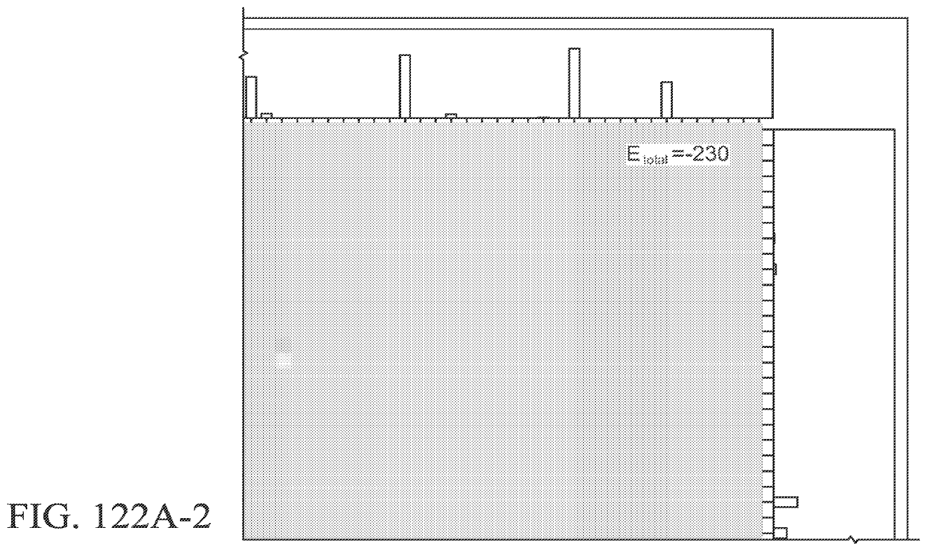

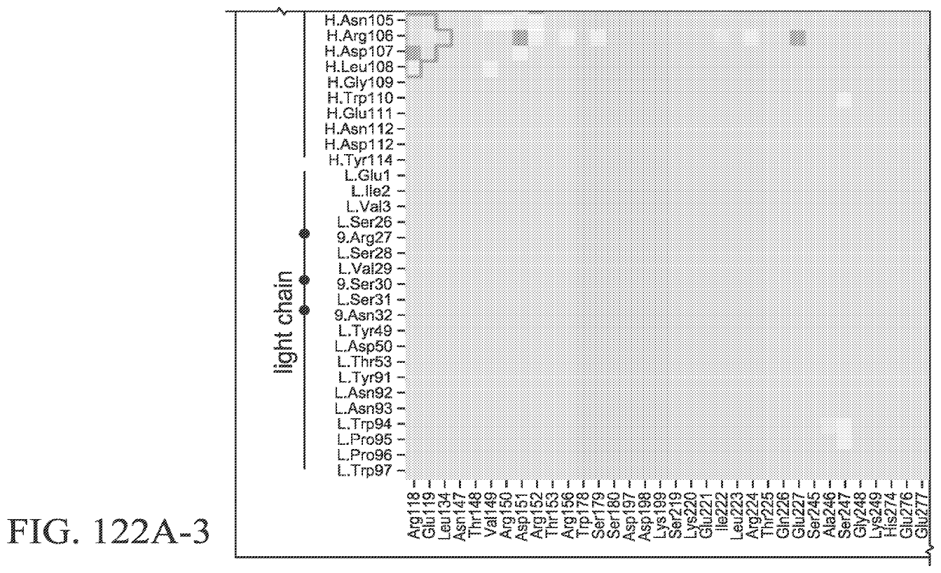

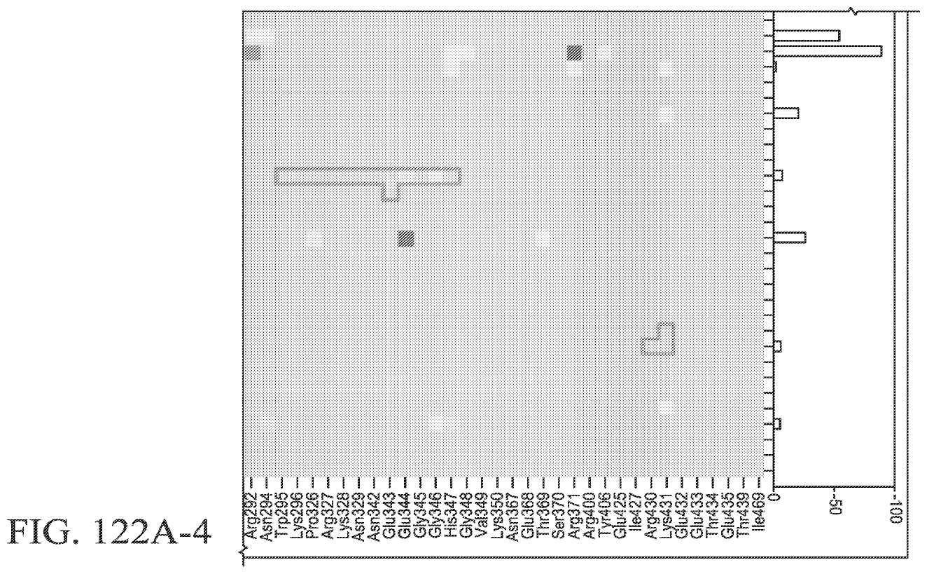

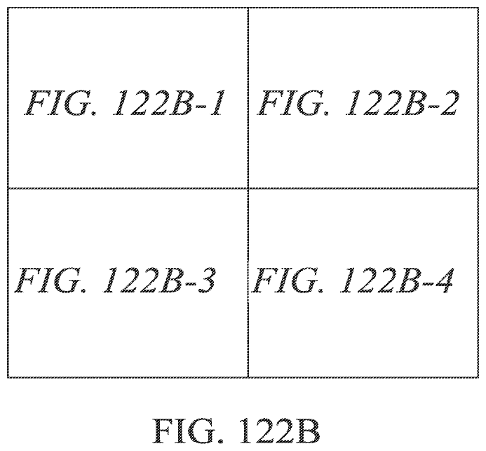

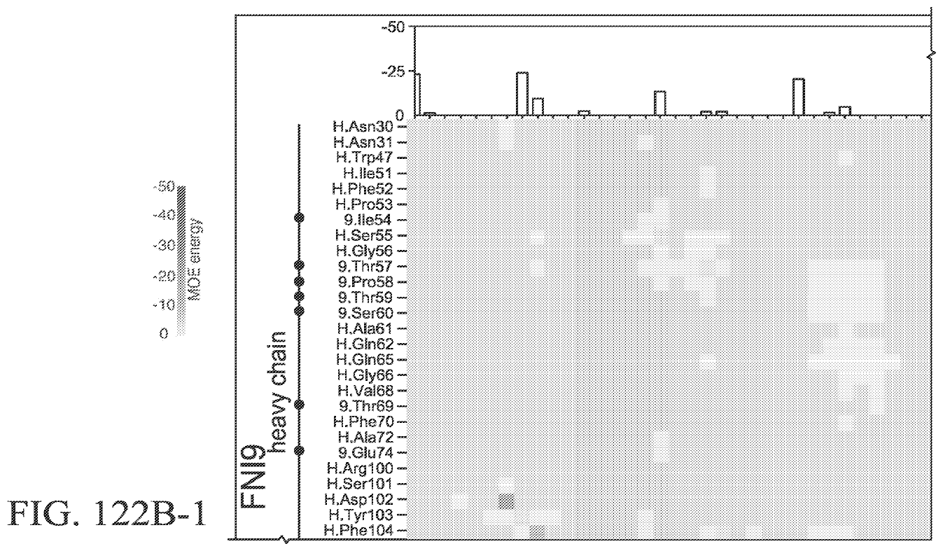

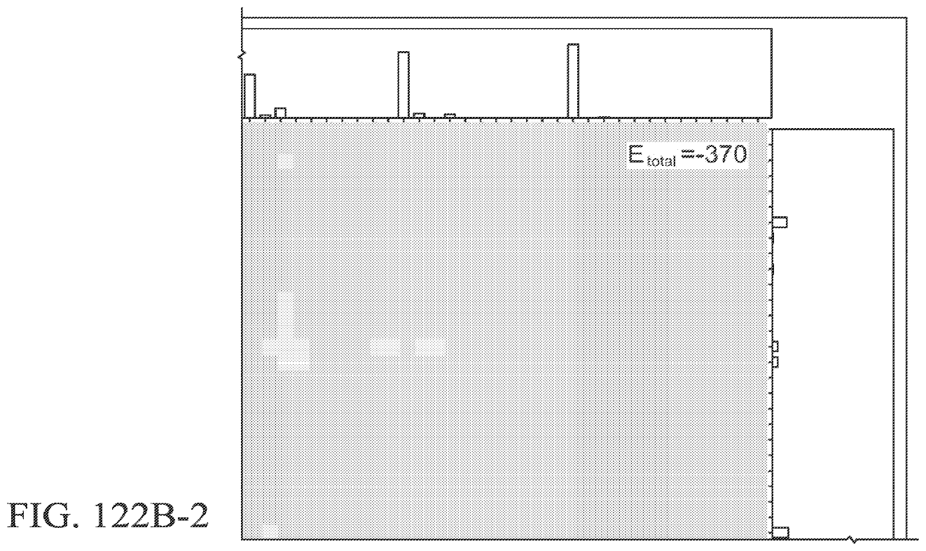

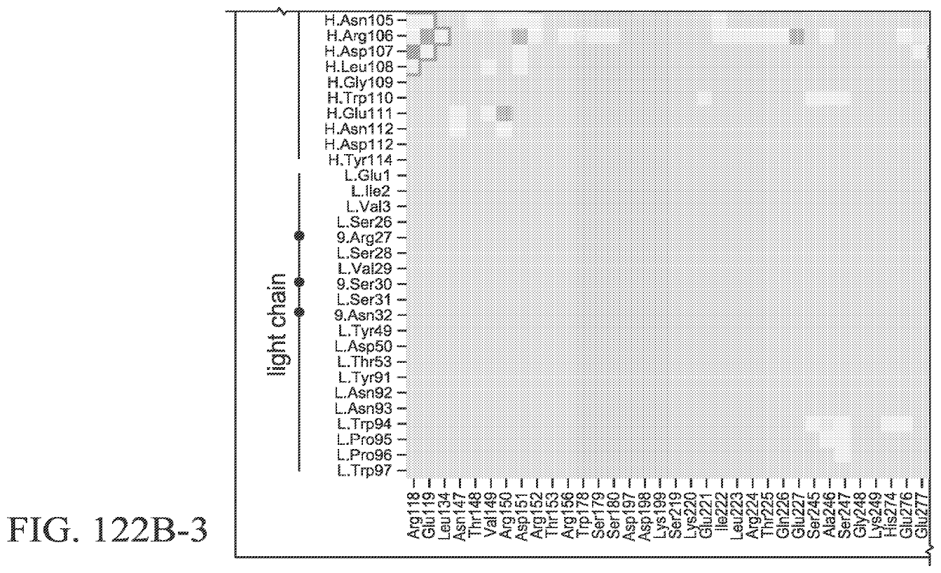

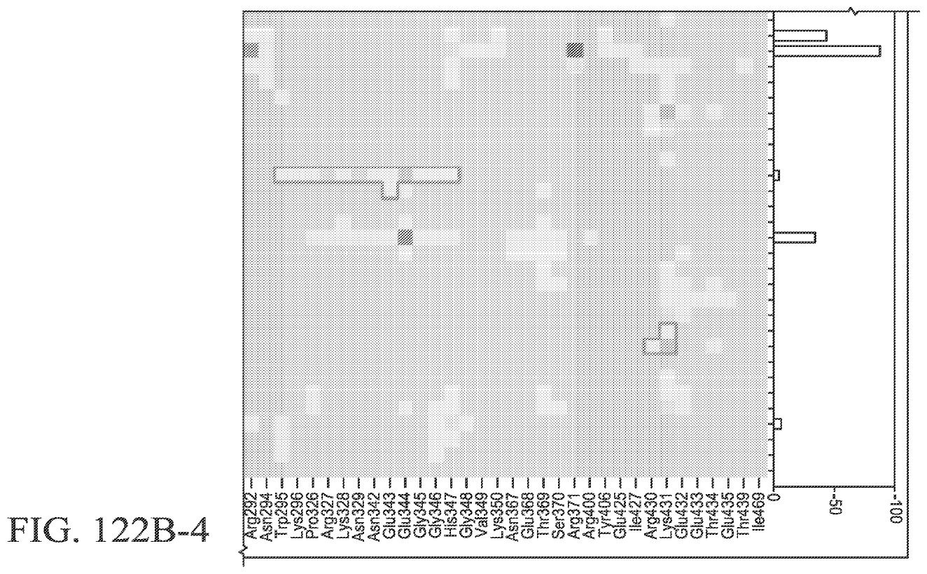

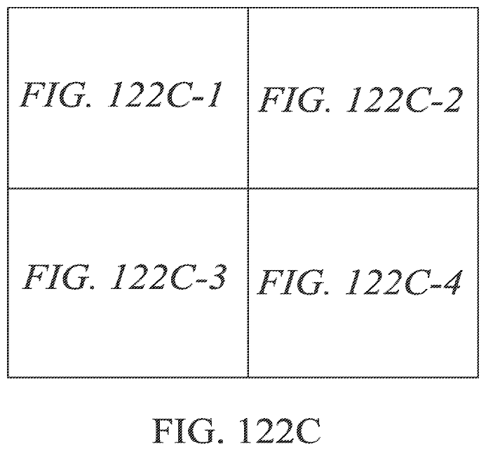

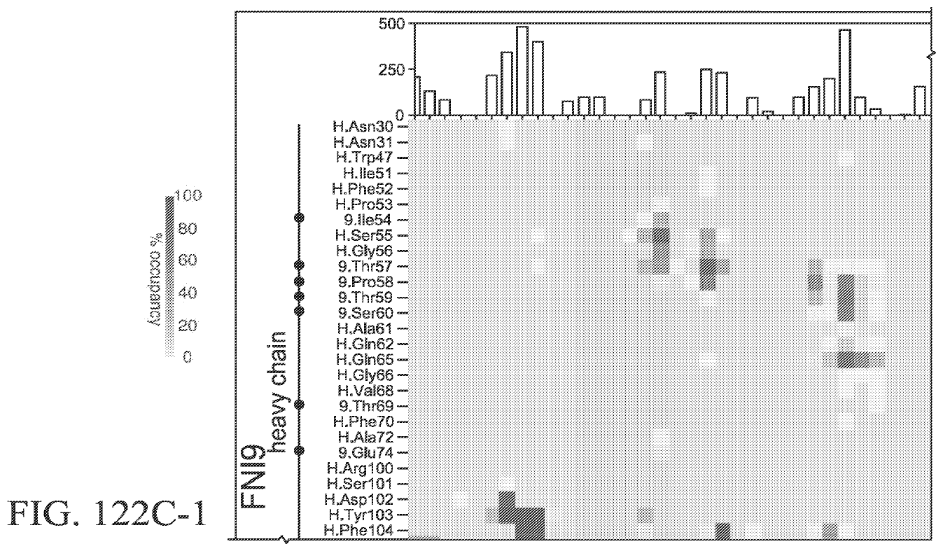

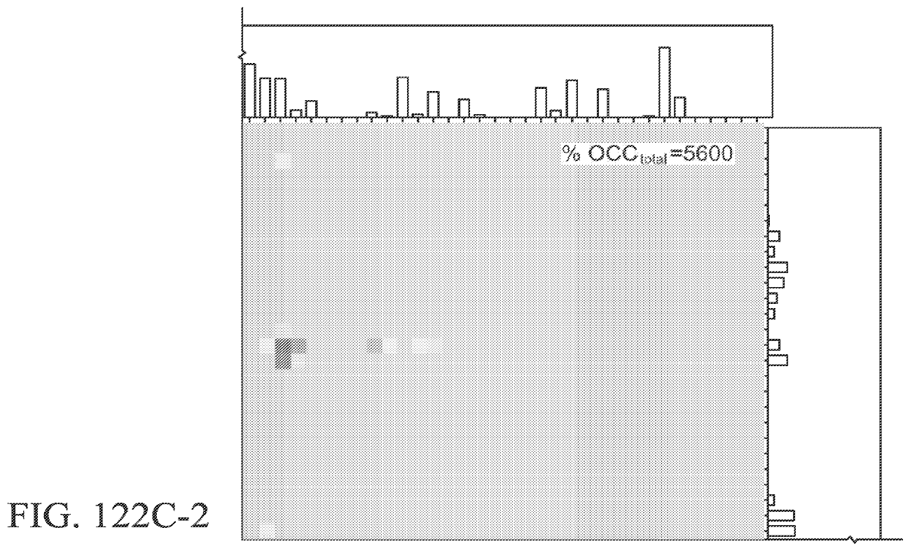

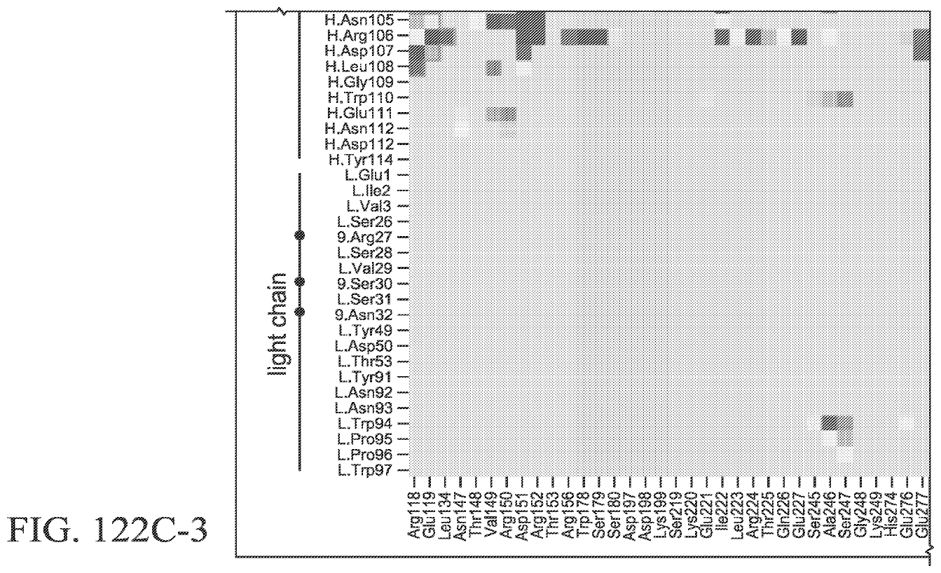

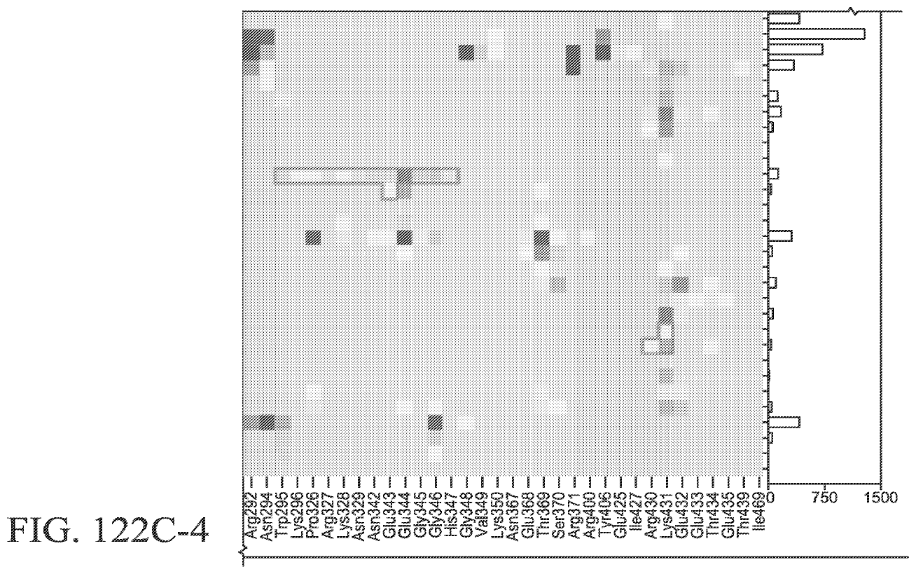

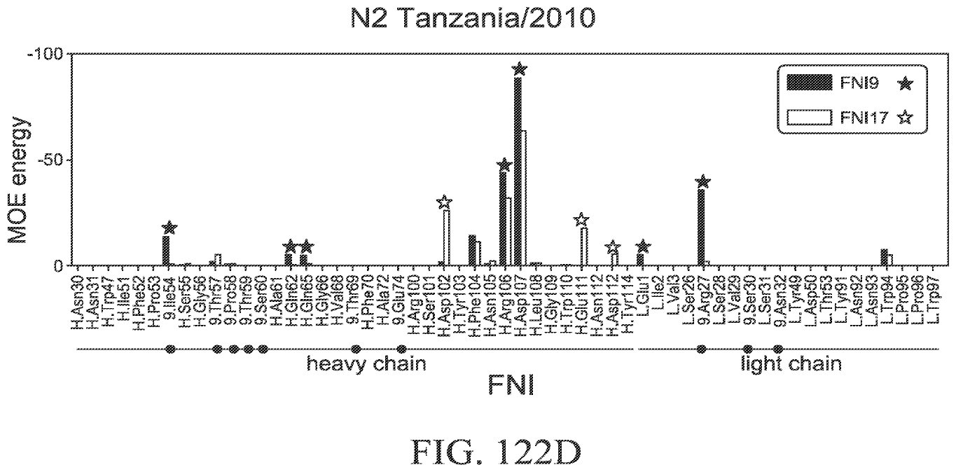

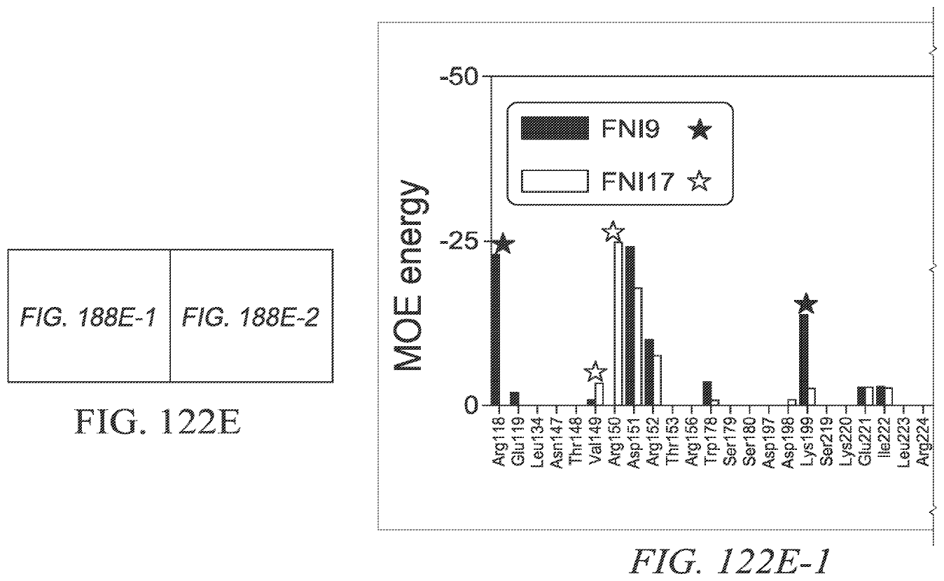

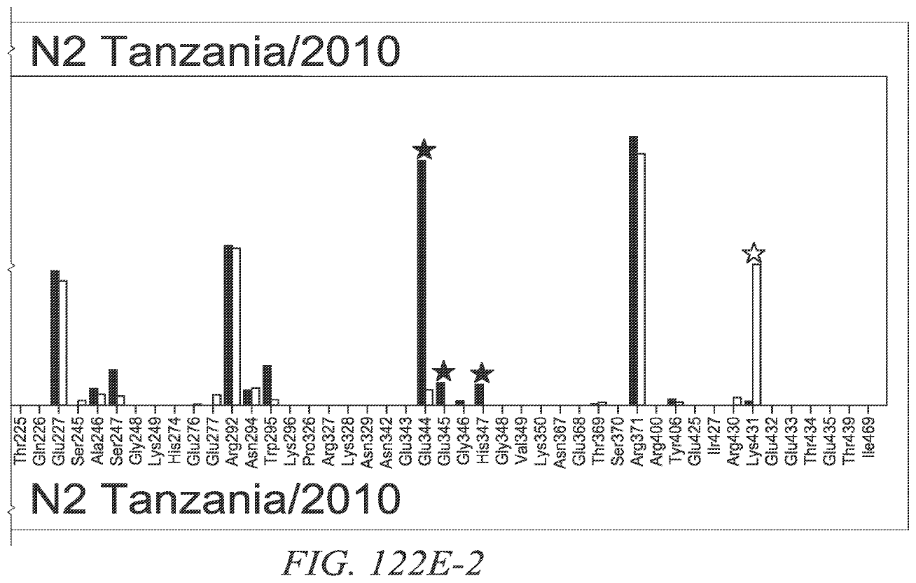

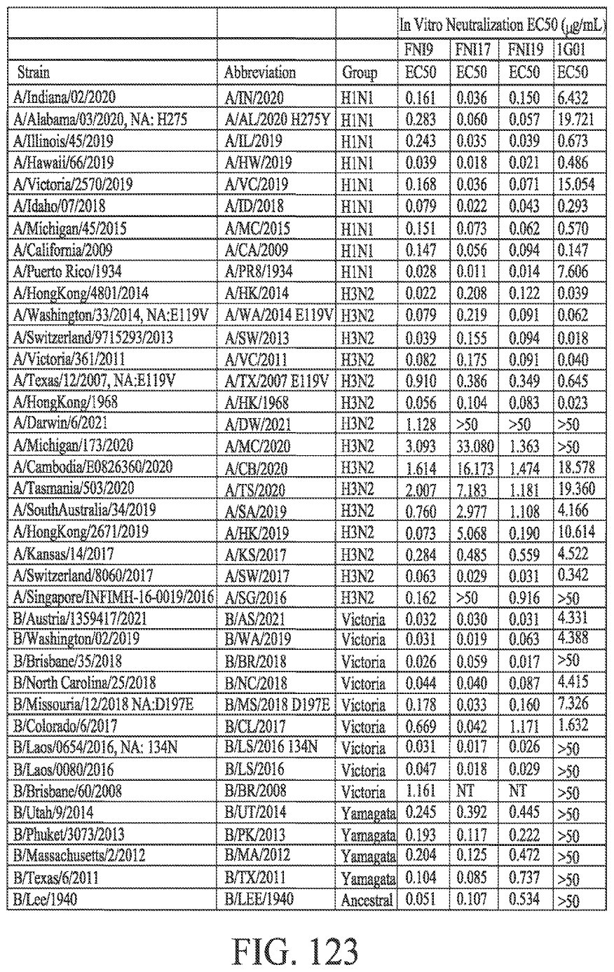

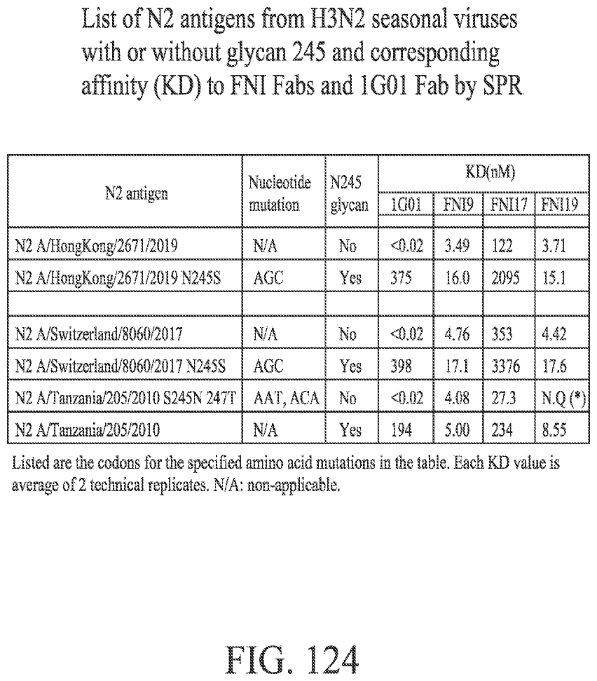

Sequence conservation was analyzed. H3N2 (IAV), H1N1 (IAV), H5N1 (IAV), H7N9 (IAV), H5N8 (IAV), H5N6 (IAV), Victoria (IBV) and Yamagata (IBV) NA protein sequences were retrieved from GISAID (www.gisaid.org). Protein sequences were aligned to a reference NA sequence using MAFFT [PMID: 23329690]. The sequences used were retrieved in October 2022, and the respective reference used for alignment was A/California/07/2009 (NC_026434.1) for H1N1 sequences, A/NewYork/392/2004 (YP 308842.1) for H3N2 sequences and 8B/Yamagata/16/1988 (AAN39803.1) for Victoria and Yamagata IBV sequences. For FIGS. 121A-121H the sequences used were retrieved in July 2022 and the respective reference used for alignment was A/NewYork/392/2004 (YP 308842.1) for H3N2, H1N1, H5N1, H7N9 H5N8, H5N6 sequences, and B/Yamagata/16/1988 (AAN39803.1) for Victoria and Yamagata IBV sequences. The multiple sequence alignments were analysed with R (https://www.R-project.org/) v.4.0.4. The logo plots were generated with R package “ggseqlogo” v.0.1 [PMID: 29036507]. The conservation per residue was computed with the R package “Biostrings” v.2.58.0 (bioconductor.org/packages/Biostrings).

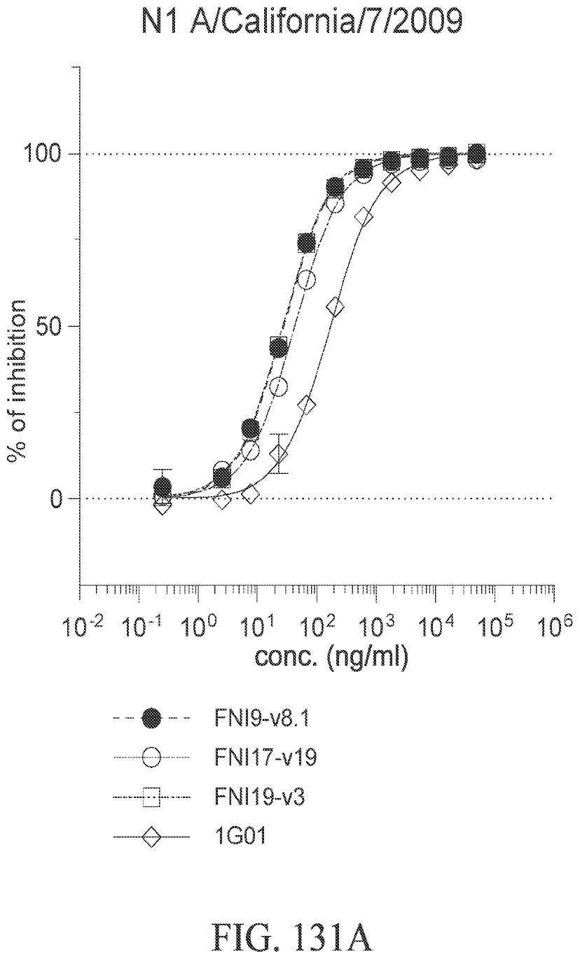

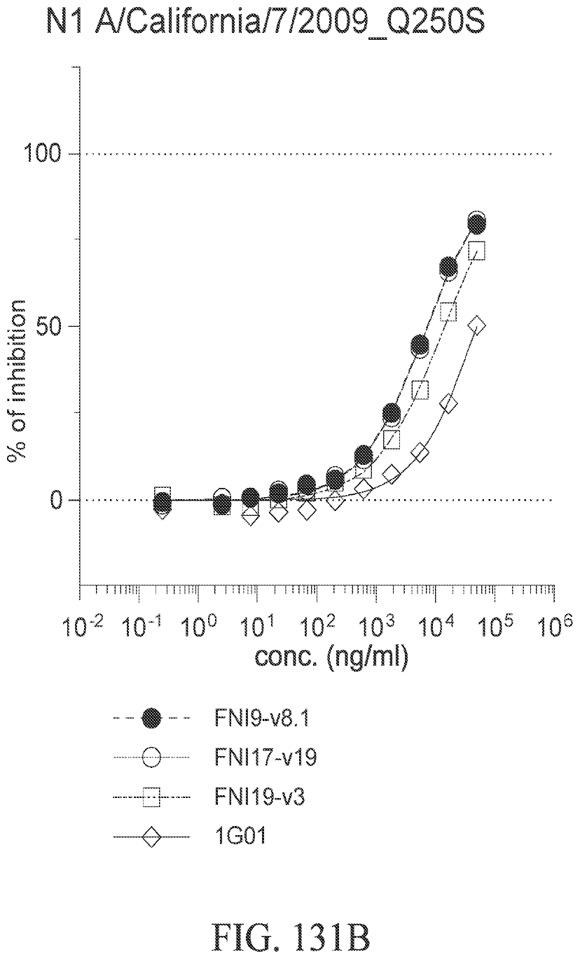

A panel of NAs with mutations identified through Deep Mutational Scanning (DMS) as capable of reducing, but not abrogating, the binding or activity of FNI mAbs was identified. Inhibition of neuraminidase enzymatic activity against the panel was measured by MUNANA assay using pseudotypes bearing the mutated NAs (FIGS. 131A-131B).

### Example 29

Prophylactic Activity of Anti-Ha Stem Antibodies can Benefit from Association with FNI9 Na-Inhibiting Antibodies

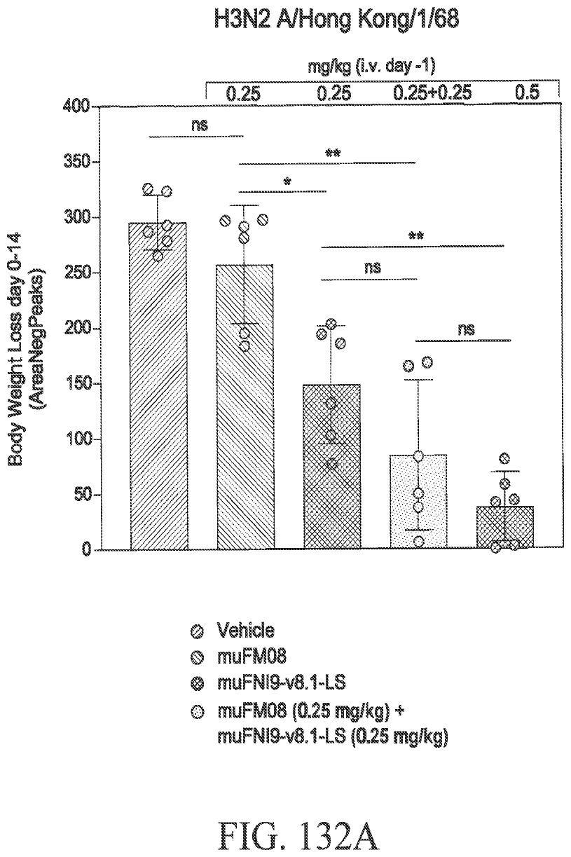

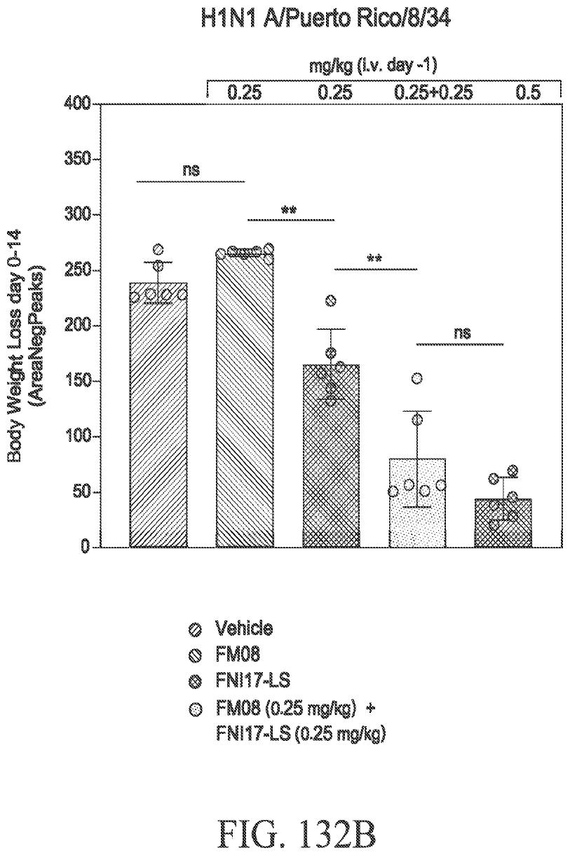

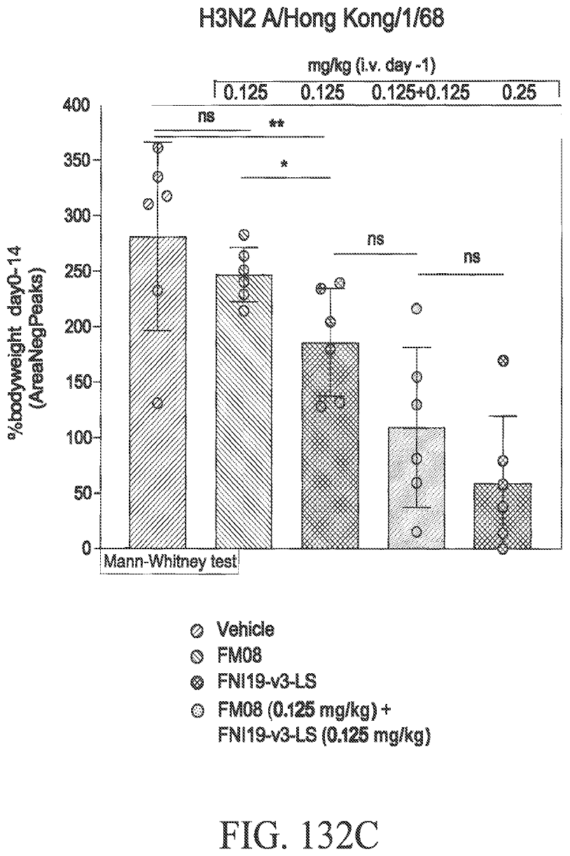

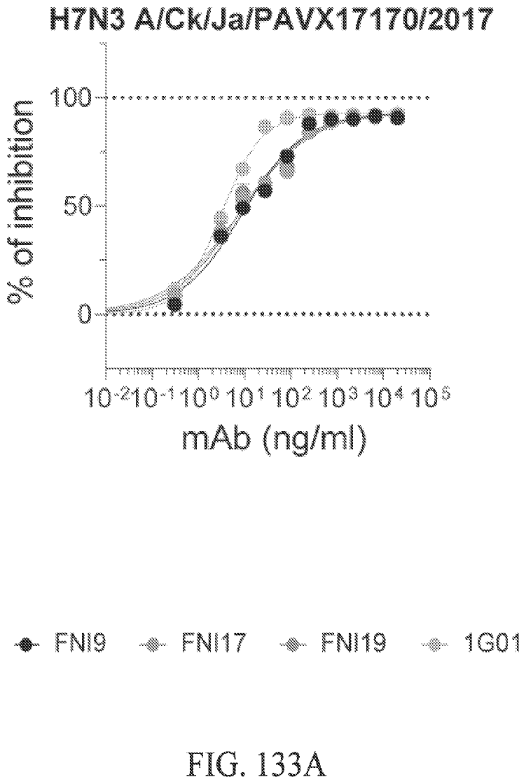

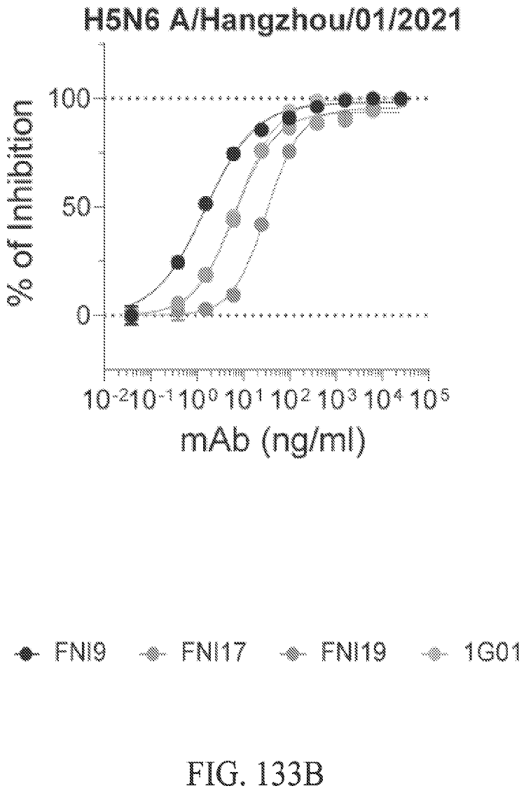

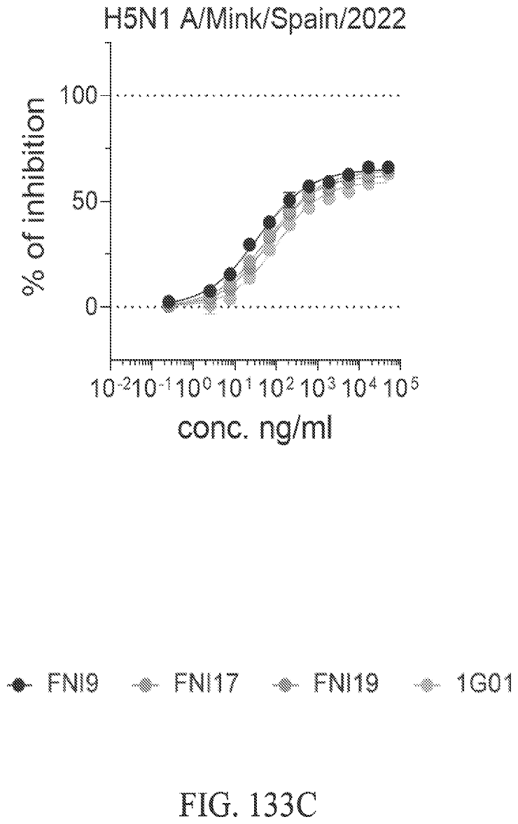

In order to evaluate the prophylactic activity of FNI antibodies in combination with anti-HA antibodies, an in vivo study was designed. Mice (BALB/c mice, 7-8 weeks of age) were prophylactically administered an ineffective dose of an anti-HA stem antibody (0.25 mg/kg or 0.125 mg/kg) in combination with an anti-NA FNI antibody at a 1:1 ratio. Mice were administered antibodies prior to infection with H3N2 A/Hong Kong/1/68 (FIGS. 132A and 132C) or H1N1 A/Puerto Rico/8/34 (FIG. 132B). Antibodies were murinized (indicated by a “mu” prefix in FIG. 132A) or human (FIGS. 132B and 132C). Anti-NA antibodies included FNI9-v8.1-LS, FNI17-LS, and FNI19-v3-LS, and the anti-HA antibody used was FM08. Antibody was administered at a dose of 0.25 mg/kg or 0.125 mg/kg, as indicated. Body weight loss in mice pre-treated with FNI antibody only or a vehicle control was also measured.

### Example 30

### Prophylactic Activity of FNI9-V8.1 and Oseltamivir Against H5N1 and H7N9 Highly Pathogenic Influenza Viruses

### Purpose

The objective of this study is to assess prophylactic efficacy of FNI9v8.1 (also referred to as “mAb-9v8.1” in the present Example) and oseltamivir on the response to H5N1 and H7N9 Highly Pathogenic Avian Influenza Viruses (HPAIV) in female mice (from a mouse model used for evaluation of anti-influenza drugs and supported by historical data for use in influenza virus disease models) from Day 0 through Day 14. The endpoints on Day 14 include clinical observations, bodyweight loss, and survival.

### Test and Control Materials

Mice in appropriate treatment groups receive a single dose of mAb-9v8.1 or vehicle control on Day −1 via intravenous injection, 24 hours prior to infection. Mice in oseltamivir treatment groups receive oseltamivir via oral gavage once daily on Days −1-5 (7 doses total) based on Day −2 individual body weight. All study animals are infected with a lethal challenge of highly pathogenic H5NT or H7N9 viruses on Day 0.

TABLE 4
Vehicle (Sterile Phosphate-Buffered Saline (PBS))

| | |
|---|---|
| Identification | Sterile PBS, pH 7, United States |
| | Pharmacopeia (USP)-grade |
| Description | Sterile PBS |
| Manufacturer | — |
| Lot Number | — |
| Stability | — |
| Retest date | — |
| Purity | — |
| Concentration | 10 mM PO₄³⁻, 137 mM NaCl, and 2.7 mM KCl |
| Preparation | Equilibrate at room temperature (RT) |
| and Handling | before administration |
| Storage | 2-8° C. |
| Dose | N/A |
| Dose Route | Intravenous (IV) injection |
| Dose Volume | Based on Day −2 individual body weight, |
| | not to exceed 250 μL |
| Administration | Single administration on Day −1 (24 hours |
| | prior to infection), based on Day −2 |
| | individual body weight |
| Safety | Minimum safety requirements include safety |
| | glasses (or equivalent), gloves, and |
| | laboratory wear at all times while handling |
| Dose Justification | N/A |

TABLE 5
Test Article 1: FNI9v8.1

| Description | Engineered human monoclonal antibody, | |
|---|---|---|
| | recombinant, purified | |
| Manufacturer | Vir Biotechnology | |
| Lot Number | — | |
| Retest Date | N/A | |
| Chemical Grade | Non-pharmaceutical grade | |
| Stability | 6 | months |
| Osmolality | 280-315 | mOsm/Kg |
| Pyrogenicity | No endotoxin detectable with | |
| | sensitivity <0.05 EU/mg | |
| Sterility | Sterile | |
| pH | 7.4 | |
| Purity | Purified by affinity chromatography | |
| Concentration | — | |
| Preparation | Handle in sterile environment | |
| and Handling | | |
| Storage | ≤−70° C. until use, then 4° C. | |
| Dose | 3 mg/kg, 0.9 mg/kg, 0.3 mg/kg | |
| Dose Route | Intravenous (IV) injection | |
| Dose Volume | 10 | mL/kg |

| | |
|---|---|
| Administration | Single administration on Day −1 (24 |
| | hours prior to Influenza infection), based |
| | on Day −2 individual body weight |
| Safety | Not hazardous, standard PPE sufficient - not |
| | for human use |
| Dose Justification | Based on previous in vivo studies |

The FN9v8.1 antibody used in this study comprises: the CDRH1-3 sequences of SEQ ID NOs: 55, 4, and 5 and the LCDR-3 sequences of SEQ ID NOs: 9, 10, and 11, respectively; the VH sequence of SEQ ID NO: 54 and the VL sequence of SEQ ID NO: 8; and the heavy chain sequence of SEQ ID NO: 107 and the light chain sequence of SEQ ID NO: 108.

TABLE 6
Test Article 2: Oseltamivir (also known as TAMIFLU ™)

| | |
|---|---|
| Description | Neuraminidase inhibitor (powder) |
| Manufacturer | MedChemExpress |
| Lot Number | HY-17016-500MG |
| Retest Date | — |
| Chemical Grade | Pharmaceutical grade |
| Stability | Up to 7 days (Study Days −1-5) at 4° C. |
| | (following reconstitution in sterile water |
| | for injection) |
| Osmolality | — |
| Sterility | Sterile |
| pH | — |
| Concentration | 1.0 mg/mL (following reconstitution in sterile |
| | water for injection) |
| Preparation | Handle in sterile environment |
| and Handling | |
| Storage | 4° C. (once resuspended) |
| Dose | 10 mg/kg |
| Dose Route | Oral gavage |
| Dose Volume | Based on Day −2 individual body weight, not |
| | to exceed 20 μL/kg as per BRT policy regarding |
| | acceptable volumes for oral gavage in mice. |
| Administration | Daily on Days −1-−5 (7 doses total), |
| | based on Day −2 individual body weight. |
| Safety | Safe - not hazardous |
| Dose Justification | Based on in vivo studies |

TABLE 7
Influenza H5N1 Virus (HPAIV Strain)

| | |
|---|---|
| Identification | Influenza |
| Supplier | — |
| Catalog Number | — |
| Lot Number | — |
| Titer | — |
| Handling | Virus aliquot(s) is/are thawed at room temperature |
| | before use. Freeze-thaws reduce the viral titer, |
| | thus each aliquot is a single-use aliquot. |
| Storage | ≤−70° C. |
| Infectious Dose | — |
| Dose volume | 50 μL/mouse |
| Route | Intranasal (i.n.) |
| Administration | Single administration on Day 0 |
| Dose Justification | This infectious dose was chosen to assess |
| | prophylactic effects of the test articles in an |
| | Influenza A model, using a level of infection that |
| | results in 90% mortality in untreated animals. |

TABLE 8
Influenza H7N9 Virus (HPAIV Strain)

| | |
|---|---|
| Identification | Influenza |
| Supplier | — |
| Catalog Number | — |
| Lot Number | — |
| Titer | — |
| Handling | Virus aliquot(s) is/are thawed at room temperature |
| | before use. Freeze-thaws reduce the viral titer, |
| | thus each aliquot is a single-use aliquot. |
| Storage | ≤−70° C. |
| Infectious Dose | — |
| Dose volume | 50 μL/mouse |
| Route | Intranasal (i.n.) |
| Administration | Single administration on Day 0 |
| Dose Justification | This infectious dose was chosen to assess prophylactic |
| | effects of the test articles in an Influenza A model, |
| | using a level of infection that results in 90% |
| | mortality in untreated animals. |

### Test System

### Animals

The mice have been used for evaluation of anti-influenza drugs and there is historical data supporting their use in influenza virus disease models.

### Methodology

### Study Outline

This study examines prophylactic efficacy of mAb-9v8.1 and oseltamivir on the response to Highly Pathogenic Avian Influenza H5N1 and H7N9 virus infection in a murine host model from Day 0 through Day 14.

The main study design uses 60 female mice (6 mice per each of 10 test groups). Extra mice are available as possible replacements for mice that are not healthy or are weight outliers prior to the experimental start date.

On Day −1, 24 hours pre-infection, mice are treated with vehicle or test article (3, 0.9, or 0.3 mg/kg dose) via intravenous injection. On Day 0, all main study animals (and any applicable extra animals that serve as replacements) are infected intranasally with a lethal challenge (LD90) of Highly Pathogenic Avian Influenza Viruses H5N1 or H7N9. Bodyweights, clinical observations, and survival are documented through Day 14.

TABLE 9
Experimental Groups, H5N1

| | No. of | | Treatment | | Intranasal | |
|---|---|---|---|---|---|---|
| Group | Mice | Test Article | Group | | Infection | Endpoints |
| 1 | 6 | — | Vehicle | | Influenza | Survival, bodyweight |
| 3 | 6 | mAb-9v8.1 | 3 | mg/kg | H5N1 (LD90) | loss, clinical |
| 4 | 6 | mAb-9v8.1 | 0.9 | mg/kg | | observations through |
| 5 | 6 | mAb-9v8.1 | 0.3 | mg/kg | | Day 14 |
| 6 | 6 | oseltamivir | 10 | mg/kg | | |
| TOTAL | 30 | | | | | |

TABLE 10
Experimental Groups, H7N9

| | No. of | | Treatment | | Intranasal | |
|---|---|---|---|---|---|---|
| Group | Mice | Test Article | Group | | Infection | Endpoints |
| 1 | 6 | — | Vehicle | | Influenza | Survival, bodyweight |
| 3 | 6 | mAb-9v8.1 | 3 | mg/kg | H7N9 (LD90) | loss, clinical |
| 4 | 6 | mAb-9v8.1 | 0.9 | mg/kg | | observations through |
| 5 | 6 | mAb-9v8.1 | 0.3 | mg/kg | | Day 14 |
| 6 | 6 | oseltamivir | 10 | mg/kg | | |
| TOTAL | 30 | | | | | |

mAb-9v8.1 treatment is administered as a single IV injection on Day −1 based on individual animal Day −2 bodyweight. Oseltamivir treatment is administered via oral gavage once daily on Days −1-−5 (7 doses total) based on Day −2 individual body weight.

### Study Procedures

### Dosing

Animals are dosed with test article or vehicle (sterile PBS) via IV injection on Day −1 (24 hours prior to infection). Antibody test articles are administered at doses of 3, 0.9, 0.3 mg/kg, not to exceed 250 μL (acceptable IV injection volumes for mice).

Animals in the oseltamivir treatment groups are administered oseltamivir via oral gavage at a dose of 10 mg/kg via oral gavage once daily on Days −1-−5 (7 doses total) based on Day −2 individual body weight.

### Daily Observations

All mice are monitored once daily from Days −2-−14.

### Body Weight Measurements

Mice are weighed on Day −2, and daily from Days 4-−14. If a mouse reaches 30% body weight loss and any of the following changes are observed, the condition is assessed by the veterinarian to determine if immediate euthanasia is warranted.
(a) Hunched posture: (i) When touched or encouraged to move, animal does not move from hunched position. (ii) When touched or encouraged to move, animal moves, but stops and returns to hunched position.
(b) When touched or encouraged to move, animal falls over and cannot right itself.
(c) When touched or encouraged to move, animal displays labored breathing.
(d) Body weight loss does not stabilize or increases over next 48 hours.

### Serum Collection for PK Analysis (Day 0)

On Day 0 (pre-infection), blood (60 μL) is collected via the saphenous vein, placed into blood collection tubes without anti-coagulant, and allowed to clot for 30-60 minutes for all animals except those in oseltamivir dose groups. Samples are then processed to serum following centrifugation at 2500×g for 5 minutes in an instrument set to room temperature. On each collection day, each serum sample is aliquoted into a fresh, labeled collection tube and snap-frozen with dry ice immediately following collection. Sample tubes are labeled and stored at <-70° C.

### Terminal Procedures

On Day 14, animals are euthanized and terminal body weights are recorded.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application Nos. 63/345,016, filed May 23, 2022; 63/427,799, filed Nov. 23, 2022; 63/430,319, filed Dec. 5, 2022; 63/481,977, filed Jan. 27, 2023; 63/488,433, filed Mar. 3, 2023; and U.S. 63/493,057 filed Mar. 30, 2023 and are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. An anti-influenza neuraminidase (anti-NA) antibody, or an antigen-binding fragment thereof, comprising (i) a heavy chain variable domain (VH) comprising a complementarity determining region (CDR)H1, a CDRH2, and a CDRH3, and (ii) and a light chain variable domain (VL) comprising a CDRL1, a CDRL2, and a CDRL3, wherein:
(a) the CDRH1 comprises or consists of the amino acid sequence set forth in SEQ ID NO:55, SEQ ID NO:3, SEQ ID NO:47, or SEQ ID NO:49; and/or
(b) the CDRH2 comprises or consists of the amino acid sequence set forth in SEQ ID NO:4, SEQ ID NO:57, or SEQ ID NO:61; and/or
(c) the CDRH3 comprises or consists of the amino acid sequence set forth in SEQ ID NO:5, SEQ ID NO:15, SEQ ID NO:51, or SEQ ID NO:53,
(d) the CDRL1 comprises or consists of the amino acid sequence set forth in SEQ ID NO:9 or SEQ ID NO:32; and/or
(e) the CDRL2 comprises or consists of the amino acid sequence set forth in SEQ ID NO:10; and/or
(f) the CDRL3 comprises or consists of the amino acid sequence set forth in SEQ ID NO:11, 18, 21, 24, 33, or 67,
optionally provided that the CDRH1, the CDRH2, the CDRH3, the CDRL1, the CDRL2, and the CDRL3 do not comprise or consist of the amino acid sequences set forth in SEQ ID NOs: (i) 3-5 and 9-11, respectively; (ii) 3, 4, 15 and 9-11, respectively; (iii) 3-5, 9, 10, and 18, respectively; (iv) 3-5, 9, 10, and 21, respectively; (v) 3-5, 9-10, and 24, respectively; or (vi) 3-5, 32, 96, and 33, respectively.

2. The antibody or antigen-binding fragment of claim 1, wherein the CDRH3 and CDRL3 comprise or consist of the amino acid sequences set forth in SEQ ID NOs: (i) 5 and 11, respectively; (ii) 5 and 18, respectively; (iii) 5 and 21, respectively; (iv) 5 and 24, respectively; (v) 5 and 33, respectively; (vi) 5 and 67, respectively; (vii) 15 and 11, respectively; (viii) 15 and 18, respectively; (ix) 15 and 21, respectively; (x) 15 and 24, respectively; (xi) 15 and 33, respectively; (xii) 15 and 67, respectively; (xiii) 51 and 11, respectively; (xiv) 51 and 18, respectively; (xv) 51 and 21, respectively; (xvi) 51 and 24, respectively; (xvii) 51 and 33, respectively; (xviii) 51 and 67, respectively; (xix) 53 and 11, respectively; (xx) 53 and 18, respectively; (xxi) 53 and 21, respectively; (xxii) 53 and 24, respectively; (xxiii) 53 and 33, respectively; or (xxiv) 53 and 67, respectively.

3. The antibody or antigen-binding fragment of claim 1 or 2, wherein the CDRH1, CDRH2, CDRH3, CDRL1, CDRL2, and CDRL3 comprise or consist of the amino acid sequences set forth in SEQ ID NOs: (i) 55, 4, 5, and 9-11, respectively; (ii) 47, 4, 5, and 9-11, respectively; (iii) 47, 4, 5, 9, 10, and 67, respectively; (iv) 49, 4, 5, and 9-11, respectively; (v) 47, 4, 5, 9, 10, and 67, respectively; (vi) 3-5, 9, 10, and 67, respectively; (vii) 55, 4, 5, 9, 10, and 67, respectively; (viii) 3, 4, 51, and 9-11, respectively; (ix) 3, 4, 51, 9, 10, and 67, respectively; (x) 55, 4, 5, and 9-11, respectively; (xi) 55, 4, 5, 9, 10, and 67, respectively; (xii) 3, 61, 5, and 9-11, respectively; (xiii) 3, 61, 5, 9, 10, and 67 respectively; (xiv) 3-5 and 9-11, respectively; or (xv) 3, 57, 5, and 9-10, respectively.

4. An anti-influenza neuraminidase (anti-NA) antibody, or an antigen-binding fragment thereof, comprising (i) a heavy chain variable domain (VH) and (ii) light chain variable domain (VL), wherein:
(i) the VH comprises the CDRH1, CDRH2, and CDRH3 of the VH amino acid sequence set forth in SEQ ID NO:54, and the VL comprises the CDRL1, CDRL2, and CDRL3 of the VL amino acid sequence set forth in SEQ ID NO:8;
(ii) the VH comprises the CDRH1, CDRH2, and CDRH3 of the VH amino acid sequence set forth in SEQ ID NO:46, and the VL comprises the CDRL1, CDRL2, and CDRL3 of the VL amino acid sequence set forth in SEQ ID NO:8;
(iii) the VH comprises the CDRH1, CDRH2, and CDRH3 of the VH amino acid sequence set forth in SEQ ID NO:46, and the VL comprises the CDRL1, CDRL2, and CDRL3 of the VL amino acid sequence set forth in SEQ ID NO:17;
(iv) the VH comprises the CDRH1, CDRH2, and CDRH3 of the VH amino acid sequence set forth in SEQ ID NO:46, and the VL comprises the CDRL1, CDRL2, and CDRL3 of the VL amino acid sequence set forth in SEQ ID NO:20;
(v) the VH comprises the CDRH1, CDRH2, and CDRH3 of the VH amino acid sequence set forth in SEQ ID NO:46, and the VL comprises the CDRL1, CDRL2, and CDRL3 of the VL amino acid sequence set forth in SEQ ID NO:23;
(vi) the VH comprises the CDRH1, CDRH2, and CDRH3 of the VH amino acid sequence set forth in SEQ ID NO:46, and the VL comprises the CDRL1, CDRL2, and CDRL3 of the VL amino acid sequence set forth in SEQ ID NO:31;
(vii) the VH comprises the CDRH1, CDRH2, and CDRH3 of the VH amino acid sequence set forth in SEQ ID NO:46, and the VL comprises the CDRL1, CDRL2, and CDRL3 of the VL amino acid sequence set forth in SEQ ID NO:66;
(viii) the VH comprises the CDRH1, CDRH2, and CDRH3 of the VH amino acid sequence set forth in SEQ ID NO:48, and the VL comprises the CDRL1, CDRL2, and CDRL3 of the VL amino acid sequence set forth in SEQ ID NO:8;
(ix) the VH comprises the CDRH1, CDRH2, and CDRH3 of the VH amino acid sequence set forth in SEQ ID NO:48, and the VL comprises the CDRL1, CDRL2, and CDRL3 of the VL amino acid sequence set forth in SEQ ID NO:17;
(x) the VH comprises the CDRH1, CDRH2, and CDRH3 of the VH amino acid sequence set forth in SEQ ID NO:48, and the VL comprises the CDRL1, CDRL2, and CDRL3 of the VL amino acid sequence set forth in SEQ ID NO:20;
(xi) the VH comprises the CDRH1, CDRH2, and CDRH3 of the VH amino acid sequence set forth in SEQ ID NO:48, and the VL comprises the CDRL1, CDRL2, and CDRL3 of the VL amino acid sequence set forth in SEQ ID NO:23;
(xii) the VH comprises the CDRH1, CDRH2, and CDRH3 of the VH amino acid sequence set forth in SEQ ID NO:48, and the VL comprises the CDRL1, CDRL2, and CDRL3 of the VL amino acid sequence set forth in SEQ ID NO:31;
(xiii) the VH comprises the CDRH1, CDRH2, and CDRH3 of the VH amino acid sequence set forth in SEQ ID NO:48, and the VL comprises the CDRL1, CDRL2, and CDRL3 of the VL amino acid sequence set forth in SEQ ID NO:66;
(xiv) the VH comprises the complementarity determining region (CDR)H1, CDRH2, and CDRH3 of the VH amino acid sequence set forth in SEQ ID NO:2, and the VL comprises the CDRL1, CDRL2, and CDRL3 of the VL amino acid sequence set forth in SEQ ID NO:66;
(xv) the VH comprises the CDRH1, CDRH2, and CDRH3 of the VH amino acid sequence set forth in SEQ ID NO:54, and the VL comprises the CDRL1, CDRL2, and CDRL3 of the VL amino acid sequence set forth in SEQ ID NO:17;
(xvi) the VH comprises the CDRH1, CDRH2, and CDRH3 of the VH amino acid sequence set forth in SEQ ID NO:54, and the VL comprises the CDRL1, CDRL2, and CDRL3 of the VL amino acid sequence set forth in SEQ ID NO:20;
(xvii) the VH comprises the CDRH1, CDRH2, and CDRH3 of the VH amino acid sequence set forth in SEQ ID NO:54, and the VL comprises the CDRL1, CDRL2, and CDRL3 of the VL amino acid sequence set forth in SEQ ID NO:23;
(xviii) the VH comprises the CDRH1, CDRH2, and CDRH3 of the VH amino acid sequence set forth in SEQ ID NO:54, and the VL comprises the CDRL1, CDRL2, and CDRL3 of the VL amino acid sequence set forth in SEQ ID NO:31;
(xix) the VH comprises the CDRH1, CDRH2, and CDRH3 of the VH amino acid sequence set forth in SEQ ID NO:54, and the VL comprises the CDRL1, CDRL2, and CDRL3 of the VL amino acid sequence set forth in SEQ ID NO:66;
(xx) the VH comprises the CDRH1, CDRH2, and CDRH3 of the VH amino acid sequence set forth in SEQ ID NO:56, and the VL comprises the CDRL1, CDRL2, and CDRL3 of the VL amino acid sequence set forth in SEQ ID NO:8;
(xxi) the VH comprises the CDRH1, CDRH2, and CDRH3 of the VH amino acid sequence set forth in SEQ ID NO:56, and the VL comprises the CDRL1, CDRL2, and CDRL3 of the VL amino acid sequence set forth in SEQ ID NO:17;
(xxii) the VH comprises the CDRH1, CDRH2, and CDRH3 of the VH amino acid sequence set forth in SEQ ID NO:56, and the VL comprises the CDRL1, CDRL2, and CDRL3 of the VL amino acid sequence set forth in SEQ ID NO:20;
(xxiii) the VH comprises the CDRH1, CDRH2, and CDRH3 of the VH amino acid sequence set forth in SEQ ID NO:56, and the VL comprises the CDRL1, CDRL2, and CDRL3 of the VL amino acid sequence set forth in SEQ ID NO:23;
(xxiv) the VH comprises the CDRH1, CDRH2, and CDRH3 of the VH amino acid sequence set forth in SEQ ID NO:54, and the VL comprises the CDRL1, CDRL2, and CDRL3 of the VL amino acid sequence set forth in SEQ ID NO:31;
(xxv) the VH comprises the CDRH1, CDRH2, and CDRH3 of the VH amino acid sequence set forth in SEQ ID NO:54, and the VL comprises the CDRL1, CDRL2, and CDRL3 of the VL amino acid sequence set forth in SEQ ID NO:66;
(xxvi) the VH comprises the CDRH1, CDRH2, and CDRH3 of the VH amino acid sequence set forth in SEQ ID NO:60, and the VL comprises the CDRL1, CDRL2, and CDRL3 of the VL amino acid sequence set forth in SEQ ID NO:8;
(xxvii) the VH comprises the CDRH1, CDRH2, and CDRH3 of the VH amino acid sequence set forth in SEQ ID NO:60, and the VL comprises the CDRL1, CDRL2, and CDRL3 of the VL amino acid sequence set forth in SEQ ID NO:17;
(xxviii) the VH comprises the CDRH1, CDRH2, and CDRH3 of the VH amino acid sequence set forth in SEQ ID NO:60, and the VL comprises the CDRL1, CDRL2, and CDRL3 of the VL amino acid sequence set forth in SEQ ID NO:20;
(xxix) the VH comprises the CDRH1, CDRH2, and CDRH3 of the VH amino acid sequence set forth in SEQ ID NO:60, and the VL comprises the CDRL1, CDRL2, and CDRL3 of the VL amino acid sequence set forth in SEQ ID NO:23;
(xxx) the VH comprises the CDRH1, CDRH2, and CDRH3 of the VH amino acid sequence set forth in SEQ ID NO:60, and the VL comprises the CDRL1, CDRL2, and CDRL3 of the VL amino acid sequence set forth in SEQ ID NO:31;
(xxxi) the VH comprises the CDRH1, CDRH2, and CDRH3 of the VH amino acid sequence set forth in SEQ ID NO:60, and the VL comprises the CDRL1, CDRL2, and CDRL3 of the VL amino acid sequence set forth in SEQ ID NO:66;
(xxxii) the VH comprises the CDRH1, CDRH2, and CDRH3 of the VH amino acid sequence set forth in SEQ ID NO:50, and the VL comprises the CDRL1, CDRL2, and CDRL3 of the VL amino acid sequence set forth in SEQ ID NO:8;
(xxxiii) the VH comprises the CDRH1, CDRH2, and CDRH3 of the VH amino acid sequence set forth in SEQ ID NO:50, and the VL comprises the CDRL1, CDRL2, and CDRL3 of the VL amino acid sequence set forth in SEQ ID NO:17;
(xxxiv) the VH comprises the CDRH1, CDRH2, and CDRH3 of the VH amino acid sequence set forth in SEQ ID NO:50, and the VL comprises the CDRL1, CDRL2, and CDRL3 of the VL amino acid sequence set forth in SEQ ID NO:20;
(xxxv) the VH comprises the CDRH1, CDRH2, and CDRH3 of the VH amino acid sequence set forth in SEQ ID NO:50, and the VL comprises the CDRL1, CDRL2, and CDRL3 of the VL amino acid sequence set forth in SEQ ID NO:23;
(xxxvi) the VH comprises the CDRH1, CDRH2, and CDRH3 of the VH amino acid sequence set forth in SEQ ID NO:50, and the VL comprises the CDRL1, CDRL2, and CDRL3 of the VL amino acid sequence set forth in SEQ ID NO:31;
(xxxvii) the VH comprises the CDRH1, CDRH2, and CDRH3 of the VH amino acid sequence set forth in SEQ ID NO:50, and the VL comprises the CDRL1, CDRL2, and CDRL3 of the VL amino acid sequence set forth in SEQ ID NO:66;
(xxxviii) the VH comprises the CDRH1, CDRH2, and CDRH3 of the VH amino acid sequence set forth in SEQ ID NO:52, and the VL comprises the CDRL1, CDRL2, and CDRL3 of the VL amino acid sequence set forth in SEQ ID NO:8;
(xxxix) the VH comprises the CDRH1, CDRH2, and CDRH3 of the VH amino acid sequence set forth in SEQ ID NO:52, and the VL comprises the CDRL1, CDRL2, and CDRL3 of the VL amino acid sequence set forth in SEQ ID NO:17;
(xxxx) the VH comprises the CDRH1, CDRH2, and CDRH3 of the VH amino acid sequence set forth in SEQ ID NO:52, and the VL comprises the CDRL1, CDRL2, and CDRL3 of the VL amino acid sequence set forth in SEQ ID NO:20;
(xxxxi) the VH comprises the CDRH1, CDRH2, and CDRH3 of the VH amino acid sequence set forth in SEQ ID NO:52, and the VL comprises the CDRL1, CDRL2, and CDRL3 of the VL amino acid sequence set forth in SEQ ID NO:23;
(xxxxii) the VH comprises the CDRH1, CDRH2, and CDRH3 of the VH amino acid sequence set forth in SEQ ID NO:52, and the VL comprises the CDRL1, CDRL2, and CDRL3 of the VL amino acid sequence set forth in SEQ ID NO:31;
(xxxxiii) the VH comprises the CDRH1, CDRH2, and CDRH3 of the VH amino acid sequence set forth in SEQ ID NO:52, and the VL comprises the CDRL1, CDRL2, and CDRL3 of the VL amino acid sequence set forth in SEQ ID NO:66;
(xxxxiv) the VH comprises the CDRH1, CDRH2, and CDRH3 of the VH amino acid sequence set forth in SEQ ID NO:2, and the VL comprises the CDRL1, CDRL2, and CDRL3 of the VL amino acid sequence set forth in SEQ ID NO:8, or
(xxxxv) the VH comprises the CDRH1, CDRH2, and CDRH3 of the VH amino acid sequence set forth in SEQ ID NO:65, and the VL comprises the CDRL1, CDRL2, and CDRL3 of the VL amino acid sequence set forth in SEQ ID NO:68,
wherein CDRH1, CDRH2, CDRH3, CDRL1, CDRL2, and CDRL3 are optionally defined in accordance with the IMGT numbering system.

5. The antibody or antigen-binding fragment of any one of claims 1-4, wherein:
(i) the VH comprises or consists of amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to, or comprises or consists of, the amino acid sequence set forth in any one of SEQ ID NOs: 54, 2, 14, 45, 46, 48, 50, 52, 56, 58, 60, 63, and 65; and
(ii) the VL comprises or consists of amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to, or comprises or consists of, the amino acid sequence set forth in any one of SEQ ID NOs:8, 17, 20, 23, 31, 37, 66, and 68,
optionally provided that the VH and VL do not comprise or consist of the amino acid sequences set forth in: (a) 2 and 8, respectively; (b) 14 and 8, respectively; (c) 2 and 17, respectively; (d) 2 and 20, respectively; (e) 2 and 23, respectively; or (f) 2 and 31, respectively.

6. The antibody or antigen-binding fragment of any one of claims 1-5, wherein the VH and VL have at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to, or comprise or consist of, the amino acid sequences set forth in SEQ ID NOs: (i) 54 and 8, respectively; (ii) 46 and 8, respectively; (iii) 46 and 17, respectively; (iv) 46 and 20, respectively; (v) 46 and 23, respectively; (vi) 46 and 31, respectively; (vii) 46 and 66, respectively; (viii) 48 and 8, respectively; (ix) 48 and 17, respectively; (x) 48 and 20, respectively; (xi) 48 and 23, respectively; (xii) 48 and 31, respectively; (xiii) 48 and 66, respectively; (xiv) 2 and 66, respectively; (xv) 54 and 17, respectively; (xvi) 54 and 20, respectively; (xvii) 54 and 23, respectively; (xviii) 54 and 31; (xix) 54 and 66, respectively; (xx) 56 and 8, respectively; (xxi) 56 and 17, respectively; (xxii) 56 and 20, respectively; (xxiii) 56 and 23, respectively; (xxiv) 54 and 31, respectively; (xxv) 54 and 66, respectively; (xxvi) 60 and 8, respectively; (xxvii) 60 and 17, respectively; (xxviii) 60 and 20, respectively; (xxix) 60 and 23, respectively; (xxx) 60 and 31, respectively; (xxxi) 60 and 66, respectively; (xxxii) 2, 14, 45, 46, 48, 50, 52, 54, 56, 58, 60, 63, or 65, and 37, respectively; (xxxiii) 2, 14, 45, 46, 48, 50, 52, 54, 56, 58, 60, 63, or 65, and 68, respectively; (xxxiv) 45, 46, 48, 50, 52, 54, 56, 58, 60, 63, or 65, and 8, respectively; (xxxv) 45, 46, 48, 50, 52, 54, 56, 58, 60, 63, or 65, and 17, respectively; (xxxvi) 45, 46, 48, 50, 52, 54, 56, 58, 60, 63, or 65, and 20, respectively; (xxxvii) 45, 46, 48, 50, 52, 54, 56, 58, 60, 63, or 65, and 23, respectively; (xxxviii) 45, 46, 48, 50, 52, 54, 56, 58, 60, 63, or 65, and 31, respectively; (xxxix) 45, 46, 48, 50, 52, 54, 56, 58, 60, 63, or 65, and 37, respectively; (xxxx) 45, 46, 48, 50, 52, 54, 56, 58, 60, 63, or 65, and 66, respectively; (xxxxi) 45, 46, 48, 50, 52, 54, 56, 58, 60, 63, or 65, and 68, respectively; (xxxxii) 2, 14, 45, 46, 48, 50, 52, 54, 56, 58, 60, 63, or 65, and 66, respectively; (xxxxiii) 2 and 8, respectively; (xxxxiv) 2 and 37, respectively; or (xxxxv) 65 and 68, respectively.

7. The antibody or antigen-binding fragment of any one of claims 1-6, wherein the VH and VL comprise or consist of the amino acid sequences set forth in SEQ ID NOs: (i) 54 and 8, respectively; (ii) 46 and 37, respectively; (iii) 48 and 37, respectively; (iv) 50 and 37, respectively; (v) 52 and 37, respectively; (vi) 54 and 37, respectively; (vii) 56 and 37, respectively; (viii) 58 and 37, respectively; (ix) 60 and 37, respectively; (x) 63 and 37, respectively; (xi) 65 and 37, respectively; (xii) 45 and 66, respectively; (xiii) 46 and 66, respectively; (xiv) 48 and 66, respectively; (xv) 50 and 66, respectively; (xvi) 52 and 66, respectively; (xvii) 54 and 66, respectively; (xviii) 56 and 66, respectively; (xix) 58 and 66, respectively; (xx) 60 and 66, respectively; (xxi) 63 and 66, respectively; (xxii) 65 and 66, respectively; (xxiii) 45 and 68, respectively; (xxiv) 46 and 68, respectively; (xxv) 48 and 68, respectively; (xxvi) 50 and 68, respectively; (xxvii) 52 and 68, respectively; (xxviii) 54 and 68, respectively; (xxix) 56 and 68, respectively; (xxx) 58 and 68, respectively; (xxxi) 60 and 68, respectively; (xxxii) 63 and 68, respectively; (xxxiii) 65 and 68, respectively; (xxxiv) 45, 46, 48, 50, 52, 54, 56, 58, 60, 63, or 65, and 8, respectively; (xxxv) 45, 46, 48, 50, 52, 54, 56, 58, 60, 63, or 65, and 17, respectively; (xxxvi) 45, 46, 48, 50, 52, 54, 56, 58, 60, 63, or 65, and 20, respectively; (xxxvii) 45, 46, 48, 50, 52, 54, 56, 58, 60, 63, or 65, and 23, respectively; (xxxviii) 45, 46, 48, 50, 52, 54, 56, 58, 60, 63, or 65, and 31, respectively; (xxxix) 2, 45, 46, 48, 50, 52, 54, 56, 58, 60, 63, or 65, and 37, respectively; (xxxx) 2, 45, 46, 48, 50, 52, 54, 56, 58, 60, 63, or 65, and 66, respectively; (xxxxi) 2, 45, 46, 48, 50, 52, 54, 56, 58, 60, 63, or 65, and 68, respectively; (xxxxii) 2, 14, 45, 46, 48, 50, 52, 54, 56, 58, 60, 63, or 65, and 66, respectively; (xxxxiii) 2 and 8, respectively; (xxxxiv) 2 and 37, respectively; (xxxxv) 45 and 37, respectively; (xxxxvi) 46 and 8, respectively; or (xxxxvii) 56 and 8, respectively.

8. The antibody or antigen-binding fragment of any one of claims 1-7, wherein the VH and VL comprise or consist of the amino acid sequences set forth in SEQ ID NOs: 54 and 8, respectively.

9. An anti-influenza neuraminidase (anti-NA) antibody, or an antigen-binding fragment thereof, comprising (i) a heavy chain variable domain (VH) comprising a complementarity determining region (CDR)H1, a CDRH2, and a CDRH3, and (ii) and a light chain variable domain (VL) comprising a CDRL1, a CDRL2, and a CDRL3, wherein:
(a) CDRH2 is as set forth in SEQ ID NO:4 and is comprised in SEQ ID NO:59, CDRH1 is as set forth in SEQ ID NO:3, and CDRH3 is as set forth in SEQ ID NO.: 5;
(b) CDRH2 is as set forth in SEQ ID NO:61 and is comprised in SEQ ID NO:62, CDRH1 is as set forth in SEQ ID NO:3, and CDRH3 is as set forth in SEQ ID NO.: 5; or
(c) CDRH2 is as set forth in SEQ ID NO:61 and is comprised in SEQ ID NO:64, CDRH1 is as set forth in SEQ ID NO:3, and CDRH3 is as set forth in SEQ ID NO:5.

10. The antibody or antigen-binding fragment of claim 9, wherein CDRL1 is as set forth in SEQ ID NO:9, CDRL2 is as set forth in SEQ ID NO:10, and CDRL3 is as set forth in any one of SEQ ID NOs:11, 18, 21, 24, 33, and 67.

11. The antibody or antigen-binding fragment of claim 10, wherein CDRL1 is as set forth in SEQ ID NO:32, CDRL2 is as set forth in SEQ ID NO:10 or 96, and CDRL3 is as set forth in any one of SEQ ID NOs:11, 18, 21, 24, 33, and 67.

12. An anti-influenza neuraminidase (anti-NA) antibody, or an antigen-binding fragment thereof, comprising a heavy chain variable domain (VH) and a light chain variable domain (VL), wherein the VH and VL comprise or consist of the amino acid sequences set forth in SEQ ID NOs: (i) 54 and 8, respectively; (ii) 46 and 8, respectively; (iii) 48 and 8, respectively; (iv) 50 and 8, respectively; (v) 52 and 8, respectively; (vi) 65 and 68, respectively; (vii) 56 and 8, respectively; (viii) 58 and 8, respectively; (ix) 60 and 8, respectively; (x) 63 and 8, respectively; (xi) 46 and 66, respectively; (xii) 48 and 66, respectively; (xiii) 50 and 66, respectively; (xiv) 52 and 66, respectively; (xv) 54 and 66, respectively; (xvi) 56 and 66, respectively; (xvii) 58 and 66, respectively; (xviii) 60 and 66, respectively; (xix) 63 and 66, respectively; or (xx) 2 and 37, respectively.

13. The antibody or antigen-binding fragment of any one of claims 1-12, wherein the influenza comprises an influenza A virus, an influenza B virus, or both.

14. The antibody or antigen-binding fragment of any one of claims 1-13, wherein the antibody, or the antigen-binding fragment, comprises a human antibody, a monoclonal antibody, a purified antibody, a single chain antibody, a Fab, a Fab′, a F(ab′)2, or Fv.

15. The antibody or antigen-binding fragment of any one of claims 1-14, wherein the antibody or antigen-binding fragment is a multi-specific antibody or antigen-binding fragment, wherein, optionally, the antibody or antigen-binding fragment is a bi-specific antibody or antigen-binding fragment.

16. The antibody or antigen-binding fragment of any one of claims 1-15, wherein the antibody or antigen-binding fragment comprises an (e.g., IgG1) Fc polypeptide or a fragment thereof.

17. The antibody or antigen-binding fragment of any one of claims 1-16, which comprises a IgG, IgA, IgM, IgE, or IgD isotype.

18. The antibody or antigen-binding fragment of any one of claims 1-17, which comprises an IgG isotype selected from IgG1, IgG2, IgG3, and IgG4, optionally with a C-terminal lysine removed or a C-terminal glycine-lysine removed.

19. The antibody or antigen-binding fragment of any one of claims 1-18, which comprises an IgG1 isotype.

20. The antibody or antigen-binding fragment of any one of claims 1-19, which comprises an IgG1m3 allotype, an IgG1m17 allotype, an IgG1 ml allotype, or any combination thereof.

21. The antibody or antigen-binding fragment of claim 16-20, wherein the Fc polypeptide or fragment thereof comprises:
(i) a mutation that increases binding affinity to a human FcRn (e.g., as measured using surface plasmon resonance (SPR) (e.g., Biacore, e.g., T200 instrument, using manufacturer's protocols)), as compared to a reference Fc polypeptide that does not comprise the mutation; and/or
(ii) a mutation that increases binding affinity to a human FcγR (e.g., as measured using surface plasmon resonance (SPR) (e.g., Biacore, e.g., T200 instrument, using manufacturer's protocols, and/or as measured using mesoscale discovery (MSD))) as compared to a reference Fc polypeptide that does not comprise the mutation.

22. The antibody or antigen-binding fragment of claim 21, wherein the mutation that increases binding affinity to a human FcRn comprises: M428L; N434S; N434H; N434A; N434S; M252Y; S254T; T256E; T250Q; P257I; Q311I; D376V; T307A; E380A; or any combination thereof.

23. The antibody or antigen-binding fragment of claim 21 or 22, wherein the mutation that increases binding affinity to a human FcRn comprises: (i) M428L/N434S; (ii) M252Y/S254T/T256E; (iii) T250Q/M428L; (iv) P257I/Q311I; (v) P257I/N434H; (vi) D376V/N434H; (vii) T307A/E380A/N434A; (viii) M428L/N434A; or (ix) any combination of (i)-(viii).

24. The antibody or antigen-binding fragment of any one of claims 21-23, wherein the mutation that increases binding affinity to a human FcRn comprises M428L/N434S or M428L/N434A.

25. The antibody or antigen-binding fragment of any one of claims 21-24, wherein the mutation that enhances binding to a FcγR comprises S239D; I332E; A330L; G236A; or any combination thereof.

26. The antibody or antigen-binding fragment of any one of claims 21-25, wherein the mutation that enhances binding to a FcγR comprises: (i) S239D/I332E; (ii) S239D/A330L/I332E; (iii) G236A/S239D/I332E; or (iv) G236A/A330L/I332E, wherein the Fc polypeptide or fragment thereof optionally comprises Ser at position 239.

27. The antibody or antigen-binding fragment of any one of claims 1-26, which comprises a mutation that alters glycosylation, wherein the mutation that alters glycosylation comprises N297A, N297Q, or N297G, and/or which is aglycosylated, and/or which is afucosylated.

28. An anti-influenza neuraminidase (anti-NA) antibody, or an antigen-binding fragment thereof, comprising
(i) in a heavy chain,
(i)(a) a variable domain (VH) comprising the complementarity determining region (CDR)H1, CDRH2, and CDRH3 amino acid sequences set forth in SEQ ID NOs:55, 4, and 5, respectively, wherein, optionally, the VH comprises an amino acid sequence having at least 90%, at least 91%, at least 92%, at least 93%, at least 94%, at least 95%, at least 96%, at least 97%, at least 98%, or at least 99% identity to, or consisting of, the amino acid sequence set forth in SEQ ID NO:54, and
(i)(b) the mutations M428L and N434S; and
(ii) in a light chain, a light chain variable domain (VL) comprising the CDRL1, CDRL2, and CDRL3 amino acid sequences set forth in SEQ ID NOs:9-11, respectively, wherein, optionally, the VL comprises an amino acid sequence having at least 90%, at least 91%, at least 92%, at least 93%, at least 94%, at least 95%, at least 96%, at least 97%, at least 98%, or at least 99% identity to, or consisting of, the amino acid sequence set forth in SEQ ID NO:8.

29. An anti-influenza neuraminidase (anti-NA) antibody, or an antigen-binding fragment thereof, comprising:
(1) (i) in a heavy chain,
(i)(a) a variable domain (VH) comprising the complementarity determining region (CDR)H1, CDRH2, and CDRH3 amino acid sequences set forth in SEQ ID NOs:47, 4, and 5, respectively, wherein, optionally, the VH comprises an amino acid sequence having at least 90%, at least 91%, at least 92%, at least 93%, at least 94%, at least 95%, at least 96%, at least 97%, at least 98%, or at least 99% identity to, or consisting of, the amino acid sequence set forth in SEQ ID NO:46, and
(i)(b) the mutations M428L and N434S; and
(ii) in a light chain, a light chain variable domain (VL) comprising the CDRL1, CDRL2, and CDRL3 amino acid sequences set forth in SEQ ID NOs:9-11, respectively, wherein, optionally, the VL comprises an amino acid sequence having at least 90%, at least 91%, at least 92%, at least 93%, at least 94%, at least 95%, at least 96%, at least 97%, at least 98%, or at least 99% identity to, or consisting of, the amino acid sequence set forth in SEQ ID NO:8,
(2) (i) in a heavy chain,
(i)(a) a variable domain (VH) comprising the complementarity determining region (CDR)H1, CDRH2, and CDRH3 amino acid sequences set forth in SEQ ID NOs:3-5, respectively, wherein, optionally, the VH comprises an amino acid sequence having at least 90%, at least 91%, at least 92%, at least 93%, at least 94%, at least 95%, at least 96%, at least 97%, at least 98%, or at least 99% identity to, or consisting of, the amino acid sequence set forth in SEQ ID NO:65, and
(i)(b) the mutations M428L and N434S; and
(ii) in a light chain, a light chain variable domain (VL) comprising the CDRL1, CDRL2, and CDRL3 amino acid sequences set forth in SEQ ID NOs:9-10, respectively, wherein, optionally, the VL comprises an amino acid sequence having at least 90%, at least 91%, at least 92%, at least 93%, at least 94%, at least 95%, at least 96%, at least 97%, at least 98%, or at least 99% identity to, or consisting of, the amino acid sequence set forth in SEQ ID NO:68; or
(3) (i) in a heavy chain,
(i)(a) a variable domain (VH) comprising the complementarity determining region (CDR)H1, CDRH2, and CDRH3 amino acid sequences set forth in SEQ ID NOs:3, 57, and 5, respectively, wherein, optionally, the VH comprises an amino acid sequence having at least 90%, at least 91%, at least 92%, at least 93%, at least 94%, at least 95%, at least 96%, at least 97%, at least 98%, or at least 99% identity to, or consisting of, the amino acid sequence set forth in SEQ ID NO:56, and
(i)(b) the mutations M428L and N434S; and
(ii) in a light chain, a light chain variable domain (VL) comprising the CDRL1, CDRL2, and CDRL3 amino acid sequences set forth in SEQ ID NOs:9-11, respectively, wherein, optionally, the VL comprises an amino acid sequence having at least 90%, at least 91%, at least 92%, at least 93%, at least 94%, at least 95%, at least 96%, at least 97%, at least 98%, or at least 99% identity to, or consisting of, the amino acid sequence set forth in SEQ ID NO:8.

30. The antibody or antigen-binding fragment of any one of claims 28-29, wherein VH and VL have at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to, or comprise or consist of, the amino acid sequences set forth in SEQ ID NOs: (i) 54 and 8, respectively; (ii) 2 and 31, respectively; (iii) 2 and 8, respectively; (iv) 46 and 8, respectively; (v) 65 and 68, respectively; or (vi) 56 and 8, respectively.

31. The antibody or antigen-binding fragment of any one of claims 28-30, wherein VH and VL comprise or consist of, the amino acid sequences set forth in SEQ ID NOs: (i) 54 and 8, respectively; (ii) 2 and 31, respectively; (iii) 2 and 8, respectively; (iv) 46 and 8, respectively; (v) 65 and 68, respectively; or (vi) 56 and 8, respectively.

32. The antibody or antigen-binding fragment of any one of claims 28-30, wherein VH and VL have at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to, or comprise or consist of, the amino acid sequences set forth in SEQ ID NOs:2 and 37, respectively.

33. The antibody or antigen-binding fragment of any one of claims 28-32, which comprises an IgG1m3 allotype, an IgG1m17 allotype, an IgG1 ml allotype, or any combination thereof.

34. The antibody or antigen-binding fragment of any one of claims 1-33, wherein the VH is comprised in a heavy chain that further comprises the CH1-CH3 amino acid sequence set forth in SEQ ID NO:34, SEQ ID NO:36, or SEQ ID NO:38, or comprises SEQ ID NO:34 with the C-terminal lysine and optionally the C-terminal glycine-lycine removed, SEQ ID NO:36 with the C-terminal glycine removed, or SEQ ID NO:38 with the C-terminal glycine removed.

35. The antibody or antigen-binding fragment of any one of claims 1-34, wherein the VL is comprised in a light chain that further comprises the CL amino acid sequence set forth in SEQ ID NO:35.

36. An anti-influenza neuraminidase (anti-NA) antibody comprising:
(1) (i) a heavy chain comprising or consisting of the amino acid sequence set forth in SEQ ID NO:39, or comprising or consisting of SEQ ID NO:39 with the C-terminal lysine removed or with the C-terminal glycine-lysine removed; and (ii) a light chain comprising or consisting of the amino acid sequence set forth in SEQ ID NO:41;
(2) (i) a heavy chain comprising or consisting of the amino acid sequence set forth in SEQ ID NO:40, or comprising or consisting of SEQ ID NO:40 with the C-terminal lysine removed or with the C-terminal glycine-lysine removed; and (ii) a light chain comprising or consisting of the amino acid sequence set forth in SEQ ID NO:41;
(3) (i) a heavy chain comprising or consisting of the amino acid sequence set forth in SEQ ID NO:42, or comprising or consisting of SEQ ID NO:42 with the C-terminal lysine removed, or with the C-terminal glycine-lysine removed; and (ii) a light chain comprising or consisting of the amino acid sequence set forth in SEQ ID NO:44;
(4) (i) a heavy chain comprising or consisting of the amino acid sequence set forth in SEQ ID NO:43, or comprising or consisting of SEQ ID NO:43 with the C-terminal lysine removed, or with the C-terminal glycine-lysine removed; and (ii) a light chain comprising or consisting of the amino acid sequence set forth in SEQ ID NO:44;
(5) (i) two heavy chains, each comprising or consisting of the amino acid sequence set forth in SEQ ID NO:39, or comprising or consisting of SEQ ID NO:39 with the C-terminal lysine removed, or with the C-terminal glycine-lysine removed; and (ii) two light chains, each comprising or consisting of the amino acid sequence set forth in SEQ ID NO:41.
(6) (i) two heavy chains, each comprising or consisting of the amino acid sequence set forth in SEQ ID NO:40, or with the C-terminal glycine-lysine removed, or comprising or consisting of SEQ ID NO:40 with the C-terminal lysine removed, or with the C-terminal glycine-lysine removed; and (ii) two light chains, each comprising or consisting of the amino acid sequence set forth in SEQ ID NO:41;
(7) (i) two heavy chains, each comprising or consisting of the amino acid sequence set forth in SEQ ID NO:42, or comprising or consisting of SEQ ID NO:42 with the C-terminal lysine removed, or with the C-terminal glycine-lysine removed; and (ii) two light chains, each chain comprising or consisting of the amino acid sequence set forth in SEQ ID NO:44; or
(8) (i) two heavy chains, each comprising or consisting of the amino acid sequence set forth in SEQ ID NO:43, or comprising or consisting of SEQ ID NO:43 with the C-terminal lysine removed, or with the C-terminal glycine-lysine removed; and (ii) two light chains, each comprising or consisting of the amino acid sequence set forth in SEQ ID NO:44.

37. An anti-influenza neuraminidase (anti-NA) antibody or antigen-binding fragment comprising the VH amino acid sequence set forth in SEQ ID NO:54 and the VL amino acid sequence set forth in SEQ ID NO:8.

38. An anti-influenza neuraminidase (anti-NA) antibody or antigen-binding fragment comprising (i) a VH comprising or consisting of the amino acid sequence of any one of SEQ ID NOs:2, 45, 46, 48, 50, 52, 54, 56, 58, 60, 63, and 65, and (ii) a VL comprising or consisting of the VL amino acid sequence of SEQ ID NO:8.

39. An anti-influenza neuraminidase (anti-NA) antibody or antigen-binding fragment comprising:
(1) (i) a VH comprising the amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSS (SEQ ID NO:54); and (ii) a VL comprising the amino acid sequence
(SEQ ID NO.: 8)
EIVMTQSPATLSLSSGERATLSCRASRSVSSNLAWYQQKPGQAPR
LLIYDASTRATGFSARFAGSGSGTEFTLTISSLQSEDSAIYYCQQ
YNNWPPWTFGQGTKVEIK;
(2) (i) a VH consisting of the amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSS (SEQ ID NO:54); and (ii) a VL consisting of the amino acid sequence
(SEQ ID NO.: 8)
EIVMTQSPATLSLSSGERATLSCRASRSVSSNLAWYQQKPGQAPR
LLIYDASTRATGFSARFAGSGSGTEFTLTISSLQSEDSAIYYCQQ
YNNWPPWTFGQGTKVEIK;
(3) (i) a heavy chain comprising the VH amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSS (SEQ ID NO:54); and (ii) a light chain comprising the VL amino acid sequence
(SEQ ID NO.: 8)
EIVMTQSPATLSLSSGERATLSCRASRSVSSNLAWYQQKPGQAPR
LLIYDASTRATGFSARFAGSGSGTEFTLTISSLQSEDSAIYYCQQ
YNNWPPWTFGQGTKVEIK,
wherein, optionally, the antibody or antigen-binding fragment is an (e.g. human) IgG1 isotype and optionally comprises M428L and N434S mutations, and/or wherein the light chain is an IgG1 kappa light chain;
(4) (i) two heavy chains each comprising the VH amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSS (SEQ ID NO:54); and (ii) two light chains each comprising the VL amino acid sequence
(SEQ ID NO.: 8)
EIVMTQSPATLSLSSGERATLSCRASRSVSSNLAWYQQKPGQAPR
LLIYDASTRATGFSARFAGSGSGTEFTLTISSLQSEDSAIYYCQQ
YNNWPPWTFGQGTKVEIK,
wherein, optionally, the antibody or antigen-binding fragment is an (e.g. human) IgG1 isotype and optionally comprises M428L and N434S mutations, and/or wherein the light chains are each an IgG1 kappa light chain;
(5) (i) a VH comprising CDRH1, CDRH2, and CDRH3 of the VH amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSS (SEQ ID NO:54), wherein, optionally, the CDRs are defined according to IMGT; and (ii) a VL comprising CDRL1, CDRL2, and CDRL3 of the VL amino acid sequence
(SEQ ID NO.: 8)
EIVMTQSPATLSLSSGERATLSCRASRSVSSNLAWYQQKPGQAPR
LLIYDASTRATGFSARFAGSGSGTEFTLTISSLQSEDSAIYYCQQ
YNNWPPWTFGQGTKVEIK,
wherein, optionally, the CDRs are defined according to IMGT;
(6) (i) a heavy chain comprising, in a VH, CDRH1, CDRH2, and CDRH3 of the VH amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSS (SEQ ID NO:54), wherein, optionally, the CDRs are defined according to IMGT; and (ii) a light chain comprising, in a VL, CDRL1, CDRL2, and CDRL3 of the VL amino acid sequence
(SEQ ID NO.: 8)
EIVMTQSPATLSLSSGERATLSCRASRSVSSNLAWYQQKPGQAPR
LLIYDASTRATGFSARFAGSGSGTEFTLTISSLQSEDSAIYYCQQ
YNNWPPWTFGQGTKVEIK,
wherein, optionally, the CDRs are defined according to IMGT, wherein, further optionally, the antibody or antigen-binding fragment is an (e.g. human) IgG1 isotype and optionally comprises M428L and N434S mutations, and/or wherein the light chain is an IgG1 kappa light chain;
(7) (i) two heavy chains each comprising, in a VH, CDRH1, CDRH2, and CDRH3 of the VH amino acid sequence QVHLVQSGAEVKEPGSSVTVSCKASGGTFNNQAISWVRQAPGQGLEWMGGIFPISGTPT SAQRFQGRVTFTADESTTTVYMDLSSLRSDDTAVYYCARAGSDYFNRDLGWENYYFAS WGQGTLVTVSS (SEQ ID NO:54) wherein, optionally, the CDRs are defined according to IMGT; and (ii) two light chains each comprising, in a VL, CDRL1, CDRL2, and CDRL3 of the VL amino acid sequence
(SEQ ID NO.: 8)
EIVMTQSPATLSLSSGERATLSCRASRSVSSNLAWYQQKPGQAPR
LLIYDASTRATGFSARFAGSGSGTEFTLTISSLQSEDSAIYYCQQ
YNNWPPWTFGQGTKVEIK,
wherein, optionally, the CDRs are defined according to IMGT, and wherein, further optionally, the antibody or antigen-binding fragment is an (e.g. human) IgG1 isotype and optionally comprises M428L and N434S mutations, and/or wherein the light chains are each an IgG1 kappa light chain; or
(8) (i) two heavy chains, wherein each of the two heavy chains comprises the VH amino acid sequence set forth in SEQ ID NO:54 and (ii) two light chains, wherein each of the two light chains comprises amino acid sequence set forth in SEQ ID NO:8, wherein, optionally, the antibody is an IgG1 isotype and wherein, further optionally, the antibody comprises M428L and N434S mutations (EU numbering).

40. The antibody or antigen-binding fragment of any one of claims 1-39, comprising in a heavy chain thereof, the amino acid mutation(s) set forth in any one of (i)-(xviii): (i) G236A, L328V, and Q295E; (ii) G236A, P230A, and Q295E; (iii) G236A, R292P, and I377N; (iv) G236A, K334A, and Q295E; (v) G236S, R292P, and Y300L; (vi) G236A and Y300L; (vii) G236A, R292P, and Y300L; (viii) G236S, G420V, G446E, and L309T; (ix) G236A and R292P; (x) R292P and Y300L; (xi) G236A and R292P; (xii) Y300L; (xiii) E345K, G236S, L235Y, and S267E; (xiv) E272R, L309T, S219Y, and S267E; (xv) G236Y; (xvi) G236W; (xvii) F243L, G446E, P396L, and S267E; (xviii) G236A, S239D, and H268E.

41. The antibody or antigen-binding fragment of any one of claims 1-40, comprising in a heavy chain thereof, the amino acid mutation G236A.

42. The antibody or antigen-binding fragment of any one of claims 1-41, which: is afucosylated; has been produced in a host cell that is incapable of fucosylation or that is inhibited in its ability to fucosylate a polypeptide; has been produced under conditions that inhibit fucosylation thereof by a host cell; or any combination thereof.

43. The antibody or antigen-binding fragment of any one of claims 1-42, which is human, humanized, or chimeric.

44. The antibody or antigen-binding fragment of any one of claims 1-43, which is capable of binding to a NA from:
(i) an influenza A virus (IAV), wherein the IAV comprises a Group 1 IAV, a Group 2 IAV, or both; and
(ii) an influenza B virus (IBV).

45. The antibody or antigen-binding fragment of claim 44, wherein:
(i) the Group 1 IAV NA comprises a N1, a N4, a N5, and/or a N8; and/or
(ii) the Group 2 IAV NA comprises a N2, a N3, a N6, a N7, and/or a N9.

46. The antibody or antigen-binding fragment of claim 45, wherein:
(i) the N1 is a N1 from any one or more of: A/California/07/2009, A/California/07/2009 I223R/H275Y, A/California/07/2009 Q250S, A/Swine/Jiangsu/J004/2018, A/Swine/Hebei/2017, A/Stockholm/18/2007, A/Brisbane/02/2018, A/Michigan/45/2015, A/Mississippi/3/2001, A/Netherlands/603/2009, A/Netherlands/602/2009, A/Vietnam/1203/2004, A/Vietnam/1203/2004 S247R, A/Vietnam/1203/2004 I223R, A/Vietnam/1203/2004 R152I, A/Vietnam/1203/2004 D199N, A/G4/SW/Shangdong/1207/2016, A/G4/SW/Henan/SN13/2018, A/G4/SW/Jiangsu/J004/2018, A/Mink/Spain/2022, and A/New Jersey/8/1976;
(ii) the N4 is from A/mallard duck/Netherlands/30/2011;
(iii) the N5 is from A/aquatic bird/Korea/CN5/2009;
(iv) the N8 is from A/harbor seal/New Hampshire/179629/2011 or A/chicken/Russia/3-29/2020;
(v) the N2 is a N2 from any one or more of: A/Washington/01/2007, A/Washington/01/2007 R292K, A/HongKong/68, A/South Australia/34/2019, A/Switzerland/8060/2017, A/Singapore/INFIMH-16-0019/2016, A/Switzerland/9715293/2013, A/Leningrad/134/17/57, A/Florida/4/2006, A/Netherlands/823/1992, A/Norway/466/2014, A/Switzerland/8060/2017, A/Texas/50/2012, A/Victoria/361/2011, A/HongKong/2671/2019, A/HongKong/2671/2019 K431E, A/SW/Mexico/SG1444/2011, A/Tanzania/205/2010, A/Aichi/2/1968, A/Bilthoven/21793/1972, A/Netherlands/233/1982, A/Shanghai/11/1987, A/Nanchang/933/1995, A/Fukui/45/2004, A/Brisbane/10/2007, A/Tasmania/503/2020 A/Cambodia/2020, A/Perth/16/2009, A/Kansas/14/2017, A/Swine/Kansas/2021, A/Canine/Korea/VC378/2012, and A/Canine/Indiana/003018/2016;
(vi) the N3 is a N3 from any one or more of A/Canada/rv504/2004 and A/Ck/Ja/2017;
(v) the N6 is a N6 from any one or more of A/swine/Ontario/01911/1/99, A/Ck/Suzhou/2019, and A/Hangzhou/2021;
(vi) the N7 is a N7 from any one or more of A/Netherlands/078/03, A/Ck/621572/03;
(vii) the N8 is from any one or more of A/harbor seal/New Hampshire/179629/2011 and A/Ck/Russia/2020; and/or
(viii) the N9 is a N9 from any one or more of: A/Anhui/2013 and A/Hong Kong/56/2015.

47. The antibody or antigen-binding fragment of any one of claims 44-46, wherein the IBV NA is a NA from any one or more of: B/Lee/10/1940 (Ancestral); B/Brisbane/60/2008 (Victoria); B/Malaysia/2506/2004 (Victoria); B/Malaysia/3120318925/2013 (Yamagata); B/Wisconsin/1/2010 (Yamagata); B/Yamanashi/166/1998 (Yamagata); B/Brisbane/33/2008; B/Colorado/06/2017; B/Hubei-wujiang/158/2009; B/Massachusetts/02/2012; B/Netherlands/234/2011; B/Perth/211/2001; B/Texas/06/2011 (Yamagata); B/Perth/211/2011; B/HongKong/05/1972; B/Phuket/3073/2013, B/Harbin/7/1994 (Victoria), B/Washington/02/2019 (Victoria); B/Victoria/504/2000 (Yamagata); B/Victoria/2/87; B/Victoria/2/87-lineage; B/Yamagata/16/88; and B/Yamagata/16/88-lineage.

48. The antibody or antigen-binding fragment of any one of claims 1-47, wherein the antibody or antigen-binding fragment is capable of binding to each of:
(i) a Group 1 IAV NA;
(ii) a Group 2 IAV NA; and
(iii) a IBV NA
with an EC₅₀ in a range from about 0.1 μg/mL to about 50 μg/mL, or in a range from about 0.1 g/mL to about 2 μg/mL, or in a range from 0.1 μg/mL to about 10 μg/mL, or in a range from 2 g/mL to about 10 μg/mL, or in a range from about 0.4 μg/mL to about 50 μg/mL, or in a range from about 0.4 μg/mL to about 2 μg/mL, or in a range from 0.4 μg/mL to about 10 μg/mL, or in a range from 2 μg/mL to about 10 μg/mL, or in a range from 0.4 μg/mL to about 1 μg/mL, or 0.4 g/mL or less, wherein, optionally,
the antibody or antigen-binding fragment is capable of binding to:
(i) the Group 1 IAV NA with an EC₅₀ in a range from about 0.4 μg/mL to about 50 g/mL, from about 0.4 μg/mL to about 10 μg/mL, from about 0.4 μg/mL to about 2 μg/mL, from about 2 μg/mL to about 50 μg/mL, from about 2 μg/mL to about 10 μg/mL, or from about 10 g/mL to about 50 μg/mL;
(ii) the Group 2 IAV NA with an EC₅₀ in a range from about 0.4 μg/mL to about 50 g/mL, or from about 0.4 μg/mL to about 10 μg/mL, or from about 0.4 μg/mL to about 2 μg/mL, or from about 2 μg/mL to about 50 μg/mL, or from about 2 μg/mL to about 10 μg/mL, or from about 10 μg/mL to about 50 μg/mL; and/or
(iii) the IBV NA with an EC₅₀ of about 0.4 μg/mL, or in a range from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL, wherein, further optionally,
the antibody or antigen-binding fragment is capable of binding to:
(i) a N1 with an EC₅₀ of about 0.4 μg/mL, or in a range from about 0.4 μg/mL to about 50 μg/mL, or in a range from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL;
(ii) a N4 with an EC₅₀ of about 0.4 μg/mL, or in a range from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL;
(iii) a N5 with an EC₅₀ in a range from about 0.4 μg/mL to about 2 μg/mL;
(iv) a N8 with an EC₅₀ of about 50 μg/mL;
(v) a N2 with an EC₅₀ in a range from about 0.4 μg/mL to about 20 μg/mL, or from about 0.4 μg/mL to about 10 μg/mL, or from about 0.4 μg/mL to about 2 μg/mL, from about 1 g/mL to about 10 μg/mL, or from about 1 μg/mL to about 20 μg/mL, or from about 1 μg/mL to about 5 μg/mL, or about 1, 2, 3, 4, 5, 6, 7, 8, 9, 10, 15, or 20 μg/mL;
(vi) a N3 with an EC₅₀ of about 0.4 μg/mL, or in a range from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL;
(vii) a N6 with an EC₅₀ of about 0.4 μg/mL, or in a range from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL;
(viii) a N7 with an EC₅₀ in a range from about 2 μg/mL to about 50 μg/mL;
(ix) a N9 with an EC₅₀ of about 0.4 μg/mL, or in a range from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL; and/or
(xi) a IBV NA with an EC₅₀ of about 0.4 μg/mL, or in a range from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL.

49. The antibody or antigen-binding fragment of claim 48, wherein the antibody or antigen-binding fragment is capable of binding to:
(i) one or more of: N1 A/California/07/2009, N1 A/California/07/2009 I223R/H275Y, N1 A/Stockholm/18/2007, N1 A/Swine/Jiangsu/J004/2008, N4 A/mallard duck/Netherlands/30/2011, N5 A/aquatic bird/Korea/CN5/2009, N2 A/Hong Kong/68, N2 A/Leningrad/134/17/57, N3 A/Canada/rv504/2004, N6 A/Swine/Ontario/01911/1/99, N9 A/Anhui/1/2013, B/Lee/10/1940 (Ancestral), B/Brisbane/60/2008 (Victoria), B/Malaysia/2506/2004 (Victoria), B/Malaysia/3120318925/2013 (Yamagata), B/Wisconsin/1/2010 (Yamagata), and B/Yamanashi/166/1998 (Yamagata), with an EC₅₀ of about 0.4 μg/mL, or in a range of from about 0.1 μg/mL to about 1.9 μg/mL, or of from about 0.1 μg/mL to about 1.5 μg/mL, or of from about 0.1 μg/mL to about 1.0 μg/mL, or about 0.1, 0.2, 0.3, 0.4, 0.5, 0.6, 0.7, 0.8, 0.9, or 1.0 μg/mL;
(ii) N5 A/aquatic bird/Korea/CN5/2009 with an EC₅₀ of about 2 μg/mL, or in a range of from about 2 μg/mL to about 10 μg/mL;
(iii) N8 A/harbor seal/New Hampshire/179629/2011 with an EC₅₀ of about 50 μg/mL;
(iv) N2 A/Washington/01/2007 with an EC₅₀ in a range from about 2 μg/mL to about g/mL;
(v) N7 A/Netherlands/078/03 with an EC₅₀ in a range from about 2 μg/mL to about 50 μg/mL;
(vi) N2 A/South Australia/34/2019 with an EC₅₀ in a range of from about 0.4 μg/mL to about 50 μg/mL;
(vii) N2 A/Switzerland/8060/2017 with an EC₅₀ in a range of from about 9.5 μg/mL to about 3.8 μg/mL;
(viii) N2 A/Singapore/INFIMH-16-0019/2016 with an EC₅₀ in a range of from about 18.4 μg/mL to about 2.2 μg/mL;
(iv) N2 A/Switzerland/9715293/2013 with an EC₅₀ in a range of from about 1.6 g/mL to about 1.2 μg/mL; and/or
(v) N1 A/Swine/Jiangsu/J004/2018 with an EC₅₀ in a range of from about 0.4 μg/mL to about 50 μg/mL, or about 0.4, about 2, about 10, or about 50 μg/mL.

50. The antibody or antigen-binding fragment of any one of claims 1-49, wherein the NA is expressed on the surface of a host cell (e.g., a CHO cell) and binding to NA is according to flow cytometry, and/or wherein the antibody or antigen-binding fragment is capable of binding to a NA with a KD of less than 1.0E-12 M, less than 1.0E-11 M, less than 1.0 E-11 M, or of 1.0E-12M or less, 1.0E-11M or less, or 1.0E-10 or less, or with a KD between 1.0E-10 and 1.0E-13, or with a KD between 1.0E-11 and 1.0E-13, wherein, optionally, the binding is as assessed by biolayer interferometry (BLI).

51. The antibody or antigen-binding fragment of claim 50, wherein the NA is a N1, a N2, and/or a N9.

52. The antibody or antigen-binding fragment of any one of claims 1-51, which is capable of binding to:
(1) (i) a NA epitope that comprises any one or more of the following amino acids (N1 NA numbering): R368, R293, E228, E344, S247, D198, D151, R118; and/or (ii) a NA epitope that comprises any one or more of the following amino acids (N2 NA numbering): R371, R292, E227, E344, S247, D198, D151, R118;
and/or
(2) (i) a NA epitope that comprises the amino acids R368, R293, E228, D151, and R118 (N1 NA numbering); and/or (ii) a NA epitope that comprises the amino acids R371, R292, E227, D151, and R118 (N2 NA numbering);
and/or
(3) an epitope comprised in or comprising a NA active site, wherein, optionally, the NA active site comprises the following amino acids (N2 numbering): R118, D151, R152, R224, E276, R292, R371, Y406, E119, R156, W178, S179, D/N198, I222, E227, H274, E277, D293, E425;
and/or
(4) an IBV NA epitope that comprises: (i) any one or more of the following amino acids: R116, D149, E226, R292, and R374; or (ii) the amino acids R116, D149, E226, R292, and R374,
wherein, optionally,
(a) the epitope further comprises any one or more of the following NA amino acids (N2 numbering): E344, E227, S247, and D198; and/or
(b) the antibody or antigen-binding fragment is capable of binding to a NA comprising a S245N amino acid mutation and/or a E221D amino acid mutation.

53. The antibody or antigen-binding fragment of any one of claims 1-52, which is capable of binding to a NA comprising a S245N amino acid mutation and/or a E221D amino acid mutation.

54. The antibody or antigen-binding fragment of any one of claims 1-53, wherein the antibody or antigen-binding fragment is capable of inhibiting a sialidase activity of (i) an IAV NA, wherein the IAV NA comprises a Group 1 IAV NA, a Group 2 IAV NA, or both, and/or of (ii) an IBV NA in an in vitro model of infection, an in vivo animal model of infection, and/or in a human, wherein, optionally:
(1) the Group 1 IAV NA comprises a H1N1 and/or a H5N1;
(2) the Group 2 IAV NA comprises a H3N2 and/or a H7N9; and/or
(3) the IBV NA comprises one or more of: B/Lee/10/1940 (Ancestral); B/HongKong/05/1972; B/Taiwan/2/1962 (Ancestral); B/Brisbane/33/2008 (Victoria); B/Brisbane/60/2008 (Victoria); B/Malaysia/2506/2004 (Victoria); B/New York/1056/2003 (Victoria); B/Florida/4/2006 (Yamagata); B/Jiangsu/10/2003 (Yamagata); B/Texas/06/2011 (Yamagata); B/Perth/211/2011; B/Harbin/7/1994 (Victoria); B/Colorado/06/2017 (Victoria); B/Washington/02/2019 (Victoria); B/Perth/211/2001 (Yamagata); B/Hubei-wujiagang/158/2009 (Yamagata); B/Wisconsin/01/2010 (Yamagata); B/Massachusetts/02/2012 (Yamagata); and B/Phuket/3073/2013 (Yamagata).

55. The antibody or antigen-binding fragment of any one of claims 1-54, wherein the antibody or antigen-binding fragment is capable of inhibiting a sialidase activity by: a Group 1 IAV NA; a Group 2 IAV NA; and/or a IBV NA,
with an IC50 in a range of
from about 0.0008 μg/mL to about 4 μg/mL, from about 0.0008 μg/mL to about 3 μg/mL, from about 0.0008 μg/mL to about 2 μg/mL, from about 0.0008 μg/mL to about 1 μg/mL, from about 0.0008 μg/mL to about 0.9 μg/mL, from about 0.0008 μg/mL to about 0.8 μg/mL, from about 0.0008 μg/mL to about 0.7 μg/mL, from about 0.0008 μg/mL to about 0.6 μg/mL, from about 0.0008 μg/mL to about 0.5 μg/mL, from about 0.0008 μg/mL to about 0.4 μg/mL, from about 0.0008 μg/mL to about 0.3 μg/mL, from about 0.0008 μg/mL to about 0.2 μg/mL, from about 0.0008 μg/mL to about 0.1 μg/mL, from about 0.0008 μg/mL to about 0.09 μg/mL, from about 0.0008 μg/mL to about 0.08 μg/mL, from about 0.0008 μg/mL to about 0.07 μg/mL, from about 0.0008 μg/mL to about 0.06 μg/mL, about 0.0008 μg/mL to about 0.05 μg/mL, about 0.0008 μg/mL to about 0.04 μg/mL, about 0.0008 μg/mL to about 0.03 μg/mL, about 0.0008 g/mL to about 0.02 μg/mL, about 0.0008 μg/mL to about 0.01 μg/mL, from 0.002 μg/mL to about 4 μg/mL, from about 0.001 μg/mL to 50 μg/mL, from about 0.1 μg/mL to about 30 μg/mL, from about 0.1 μg/mL to about 20 μg/mL, from about 0.1 μg/mL to about 10 μg/mL, from about 0.1 μg/mL to about 9 μg/mL, from about 0.1 μg/mL to about 8 μg/mL, from about 0.1 μg/mL to about 7 μg/mL, from about 0.1 μg/mL to about 6 μg/mL, from about 0.1 μg/mL to about 5 g/mL, from about 0.1 μg/mL to about 4 μg/mL, from about 0.1 μg/mL to about 3 μg/mL, from about 0.1 μg/mL to about 2 μg/mL, from about 0.1 μg/mL to about 1 μg/mL, from about 0.1 g/mL to about 0.9 μg/mL, from about 0.1 μg/mL to about 0.8 μg/mL, from about 0.1 μg/mL to about 0.7 μg/mL, from about 0.1 μg/mL to about 0.6 μg/mL, from about 0.1 μg/mL to about 0.5 g/mL, from about 0.1 μg/mL to about 0.4 μg/mL, from about 0.1 μg/mL to about 0.3 μg/mL, from about 0.1 μg/mL to about 0.2 μg/mL, from about 0.8 μg/mL to about 30 μg/mL, from about 0.8 μg/mL to about 20 μg/mL, from about 0.8 μg/mL to about 10 μg/mL, from about 0.8 μg/mL to about 9 μg/mL, from about 0.8 μg/mL to about 8 μg/mL, from about 0.8 μg/mL to about 7 g/mL, from about 0.8 μg/mL to about 6 μg/mL, from about 0.8 μg/mL to about 5 μg/mL, from about 0.8 μg/mL to about 4 μg/mL, from about 0.8 μg/mL to about 3 μg/mL, from about 0.8 g/mL to about 2 μg/mL, from about 0.8 μg/mL to about 1 μg/mL, or of about 0.1 μg/mL, about 0.2 μg/mL, about 0.3 μg/mL, about 0.4 μg/mL, about 0.5 μg/mL, about 0.6 μg/mL, about 0.7 g/mL, about 0.8 μg/mL, about 0.9 μg/mL, about 1.0 μg/mL, about 1.5 μg/mL, about 2.0 μg/mL, about 2.5 μg/mL, about 3.0 μg/mL, about 3.5 μg/mL, about 4.0 μg/mL, about 4.5 μg/mL, about 5.0 μg/mL, about 5.5 μg/mL, about 6.0 μg/mL, about 6.5 μg/mL, about 7.0 μg/mL, about 7.5 g/mL, about 8.0 μg/mL, about 8.5 μg/mL, about 9.0 μg/mL, about 10 μg/mL, about 11 μg/mL, about 12 μg/mL, about 13 μg/mL, about 14 μg/mL, about 15 μg/mL, about 16 μg/mL, about 17 g/mL, about 18 μg/mL, about 19 μg/mL, about 20 μg/mL, about 25 μg/mL, and/or about 30 g/mL, wherein, optionally,
the antibody or antigen-binding fragment is capable of inhibiting NA sialidase activity of one or more Group 1 and/or Group 2 IAV, and/or of one or more IBV, with an IC50 in a range of from: about 0.00001 μg/ml to about 25 μg/ml, or about 0.0001 μg/ml to about 10 μg/ml, or about 0.0001 μg/ml to about 1 μg/ml, or about 0.0001 μg/ml to about 0.1 μg/ml, or about 0.0001 μg/ml to about 0.01 μg/ml, or about 0.0001 μg/ml to about 0.001 μg/ml, or about 0.0001 μg/ml to about 0.0001 μg/ml, or about 0.0001 μg/ml to about 25 μg/ml, or about 0.0001 μg/ml to about 10 μg/ml, or about 0.0001 μg/ml to about 1 μg/ml, or about 0.0001 μg/ml to about 0.1 μg/ml, or about 0.0001 μg/ml to about 0.01 μg/ml, or about 0.001 μg/ml to about 25 μg/ml, or about 0.001 μg/ml to about 10 μg/ml, or about 0.001 μg/ml to about 1 μg/ml, or about 0.001 μg/ml to about 0.1 μg/ml, or about 0.001 μg/ml to about 0.01 μg/ml, or about 0.01 μg/ml to about 25 μg/ml, or about 0.01 μg/ml to about 10 μg/ml, or about 0.01 μg/ml to about 1 μg/ml, or about 0.01 μg/ml to about 0.1 μg/ml, or about 1 μg/ml to about 25 μg/ml, or about 1 μg/ml to about 10 μg/ml,
or of about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 μg/ml.

56. The antibody or antigen-binding fragment of any one of claims 1-55, which is capable of activating a human FcγRIIIa, wherein, optionally,
activation is as determined using a host cell (optionally, a Jurkat cell) comprising: (i) the human FcγRIIIa (optionally, a F158 allele); and (ii) a NFAT expression control sequence operably linked to a sequence encoding a reporter, such as a luciferase reporter, following incubation (e.g., of 23 hours) of the antibody or antigen-binding fragment with a target cell (e.g., a A549 cell) infected with a IAV, wherein, further optionally,
activation is as determined following an incubation (optionally, for about 23 hours) of the antibody or antigen-binding fragment with the target cell infected with a H1N1 IAV, wherein, optionally, the H1N1 IAV is A/PR8/34, and/or wherein, optionally, the infection has a multiplicity of infection (MOI) of 6.

57. The antibody or antigen-binding fragment of any one of claims 1-56, which is capable of neutralizing infection by an IAV and/or an IBV, wherein, optionally, the IAV and/or the IBV is antiviral-resistant, wherein, optionally, the antiviral is oseltamivir.

58. The antibody or antigen-binding fragment of any one of claims 44-57, wherein: (1) the IAV comprises a N1 NA that comprises the amino acid mutation(s): H275Y; E119D+H275Y; S247N+H275Y; I222V; and/or N294S, wherein, optionally, the IAV comprises CA09 or A/Aichi; and/or (2) the IAV comprises a N2 NA that comprises the amino acid mutation(s) E119V, Q136K, and/or R292K.

59. The antibody or antigen-binding fragment of any one of claims 1-58, wherein the antibody or antigen-binding fragment is capable of: (1) treating and/or preventing (i) an IAV infection and/or (ii) an IBV infection, in a subject; and/or (2) treating and/or attenuating an infection by: (i) a H1N1 virus, wherein, optionally, the H1N1 virus comprises A/PR8/34; and/or (ii) a H3N2 virus, wherein, optionally, the H3N2 virus optionally comprises A/Hong Kong/68; and/or (3) preventing weight loss in a subject infected by the IAV and/or IBV, optionally for (i) up to 15 days, or (ii) more than 15 days, following administration of an effective amount of the antibody or antigen-binding fragment; and/or (4) preventing a loss in body weight of greater than 10% in a subject having an IAV infection and/or an IBV infection, as determined by reference to the subject's body weight just prior to the IAV and/or IBV infection; and/or (5) extending survival of a subject having an IAV infection and/or an IBV infection.

60. The antibody or antigen-binding fragment of any one of claims 1-59, wherein the antibody or antigen-binding fragment has an in vivo half-life in a mouse (e.g., a tg32 mouse):
(i) in a range of from: about 10 days to about 14 days, about 10.2 days to about 13.8 days, about 10.5 days to about 13.5 days, about 11 days to about 13 days, about 11.5 days to about 12.5 days, between 10 days and 14 days, or between 10.5 days and 13.5 days, or between 11 days and 13 days, or of about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, or 14.0 days; or
(ii) in a range of from about 12 days to about 16 days, about 12.5 days to 15.5 days, about 13 days to 15 days, about 13.5 days to about 14.5 days, or between 12 days and 16 days, or between 13 days and 15 days, or between 13.5 days and 14.5 days, or of about 12.0, 12.1, 12.2, 12.3, 12.4, 12.5, 12.6, 12.7, 12.8, 12.9, 13.0, 13.1, 13.2, 13.3, 13.4, 13.5, 1.36, 13.7, 13.8, 13.9, 14.0, 14.1, 14.2, 14.3, 14.4, 14.5, 14.6, 14.7, 14.8, 14.9, 15.0 15.1, 15.2, 15.3, 15.4, 15.5, 1.56, 15.7, 15.8, 15.9, or 16.0 days.

61. The antibody or antigen-binding fragment of claim 59 or 60, wherein the antibody or antigen-binding fragment is capable of binding to a neuraminidase (NA) from: (i) an influenza A virus (IAV), wherein the IAV comprises a Group 1 IAV, a Group 2 IAV, or both; and/or (ii) an influenza B virus (IBV), and wherein, optionally, the antibody or antigen-binding fragment is capable of (1) inhibiting NA sialidase activity and/or (2) neutralizing infection by the IAV and/or IBV.

62. An antibody comprising:
(1) (i) a heavy chain comprising the amino acid sequence
(SEQ ID NO.: 107)
QVHLVQSGAEVKEPGSSVTVSCKASGGTFNNQAISWVRQAPGQGL
EWMGGIFPISGTPTSAQRFQGRVTFTADESTTTVYMDLSSLRSDD
TAVYYCARAGSDYFNRDLGWENYYFASWGQGTLVTVSSASTKGPS
VFPLAPSSKSTSGGTAALGCLVKDYFPEPVTVSWNSGALTSGVHT
FPAVLQSSGLYSLSSVVTVPSSSLGTQTYICNVNHKPSNTKVDKR
VEPKSCDKTHTCPPCPAPELLGGPSVFLFPPKPKDTLMISRTPEV
TCVVVDVSHEDPEVKFNWYVDGVEVHNAKTKPREEQYNSTYRVVS
VLTVLHQDWLNGKEYKCKVSNKALPAPIEKTISKAKGQPREPQVY
TLPPSREEMTKNQVSLTCLVKGFYPSDIAVEWESNGQPENNYKTT
PPVLDSDGSFFLYSKLTVDKSRWQQGNVFSCSVLHEALHSHYTQK
SLSLSPGK,
or SEQ ID NO:107 with the C-terminal lysine or the C-terminal glycine-lysine removed; and
(ii) a light chain comprising the amino acid sequence
(SEQ ID NO.: 108)
EIVMTQSPATLSLSSGERATLSCRASRSVSSNLAWYQQKPGQAPR
LLIYDASTRATGFSARFAGSGSGTEFTLTISSLQSEDSAIYYCQQ
YNNWPPWTFGQGTKVEIKRTVAAPSVFIFPPSDEQLKSGTASVVC
LLNNFYPREAKVQWKVDNALQSGNSQESVTEQDSKDSTYSLSSTL
TLSKADYEKHKVYACEVTHQGLSSPVTKSFNRGEC;
(2) (i) a heavy chain consisting of the amino acid sequence
(SEQ ID NO.: 107)
QVHLVQSGAEVKEPGSSVTVSCKASGGTFNNQAISWVRQAPGQGL
EWMGGIFPISGTPTSAQRFQGRVTFTADESTTTVYMDLSSLRSDD
TAVYYCARAGSDYFNRDLGWENYYFASWGQGTLVTVSSASTKGPS
VFPLAPSSKSTSGGTAALGCLVKDYFPEPVTVSWNSGALTSGVHT
FPAVLQSSGLYSLSSVVTVPSSSLGTQTYICNVNHKPSNTKVDKR
VEPKSCDKTHTCPPCPAPELLGGPSVFLFPPKPKDTLMISRTPEV
TCVVVDVSHEDPEVKFNWYVDGVEVHNAKTKPREEQYNSTYRVVS
VLTVLHQDWLNGKEYKCKVSNKALPAPIEKTISKAKGQPREPQVY
TLPPSREEMTKNQVSLTCLVKGFYPSDIAVEWESNGQPENNYKTT
PPVLDSDGSFFLYSKLTVDKSRWQQGNVFSCSVLHEALHSHYTQK
SLSLSPGK,
or SEQ ID NO:107 with the C-terminal lysine or the C-terminal glycine-lysine removed; and
(ii) a light chain consisting of the amino acid sequence
(SEQ ID NO.: 108)
EIVMTQSPATLSLSSGERATLSCRASRSVSSNLAWYQQKPGQAPR
LLIYDASTRATGFSARFAGSGSGTEFTLTISSLQSEDSAIYYCQQ
YNNWPPWTFGQGTKVEIKRTVAAPSVFIFPPSDEQLKSGTASVVC
LLNNFYPREAKVQWKVDNALQSGNSQESVTEQDSKDSTYSLSSTL
TLSKADYEKHKVYACEVTHQGLSSPVTKSFNRGEC;
(3) (i) two heavy chains, wherein each of the two heavy chains comprises the amino acid sequence
(SEQ ID NO.: 107)
QVHLVQSGAEVKEPGSSVTVSCKASGGTFNNQAISWVRQAPGQGL
EWMGGIFPISGTPTSAQRFQGRVTFTADESTTTVYMDLSSLRSDD
TAVYYCARAGSDYFNRDLGWENYYFASWGQGTLVTVSSASTKGPS
VFPLAPSSKSTSGGTAALGCLVKDYFPEPVTVSWNSGALTSGVHT
FPAVLQSSGLYSLSSVVTVPSSSLGTQTYICNVNHKPSNTKVDKR
VEPKSCDKTHTCPPCPAPELLGGPSVFLFPPKPKDTLMISRTPEV
TCVVVDVSHEDPEVKFNWYVDGVEVHNAKTKPREEQYNSTYRVVS
VLTVLHQDWLNGKEYKCKVSNKALPAPIEKTISKAKGQPREPQVY
TLPPSREEMTKNQVSLTCLVKGFYPSDIAVEWESNGQPENNYKTT
PPVLDSDGSFFLYSKLTVDKSRWQQGNVFSCSVLHEALHSHYTQK
SLSLSPGK,
or SEQ ID NO:107 with the C-terminal lysine or the C-terminal glycine-lysine removed; and
(ii) two light chains, wherein each of the two light chains comprises the amino acid sequence
(SEQ ID NO.: 108)
EIVMTQSPATLSLSSGERATLSCRASRSVSSNLAWYQQKPGQAPR
LLIYDASTRATGFSARFAGSGSGTEFTLTISSLQSEDSAIYYCQQ
YNNWPPWTFGQGTKVEIKRTVAAPSVFIFPPSDEQLKSGTASVVC
LLNNFYPREAKVQWKVDNALQSGNSQESVTEQDSKDSTYSLSSTL
TLSKADYEKHKVYACEVTHQGLSSPVTKSFNRGEC;
(4) (i) two heavy chains, wherein each of the two heavy chains consists of the amino acid sequence
(SEQ ID NO.: 107)
QVHLVQSGAEVKEPGSSVTVSCKASGGTFNNQAISWVRQAPGQGL
EWMGGIFPISGTPTSAQRFQGRVTFTADESTTTVYMDLSSLRSDD
TAVYYCARAGSDYFNRDLGWENYYFASWGQGTLVTVSSASTKGPS
VFPLAPSSKSTSGGTAALGCLVKDYFPEPVTVSWNSGALTSGVHT
FPAVLQSSGLYSLSSVVTVPSSSLGTQTYICNVNHKPSNTKVDKR
VEPKSCDKTHTCPPCPAPELLGGPSVFLFPPKPKDTLMISRTPEV
TCVVVDVSHEDPEVKFNWYVDGVEVHNAKTKPREEQYNSTYRVVS
VLTVLHQDWLNGKEYKCKVSNKALPAPIEKTISKAKGQPREPQVY
TLPPSREEMTKNQVSLTCLVKGFYPSDIAVEWESNGQPENNYKTT
PPVLDSDGSFFLYSKLTVDKSRWQQGNVFSCSVLHEALHSHYTQK
SLSLSPGK,
or SEQ ID NO:107 with the C-terminal lysine or the C-terminal glycine-lysine removed; and
(ii) two light chains, wherein each of the two light chains consists of the amino acid sequence
(SEQ ID NO.: 108)
EIVMTQSPATLSLSSGERATLSCRASRSVSSNLAWYQQKPGQAPR
LLIYDASTRATGFSARFAGSGSGTEFTLTISSLQSEDSAIYYCQQ
YNNWPPWTFGQGTKVEIKRTVAAPSVFIFPPSDEQLKSGTASVVC
LLNNFYPREAKVQWKVDNALQSGNSQESVTEQDSKDSTYSLSSTL
TLSKADYEKHKVYACEVTHQGLSSPVTKSFNRGEC.

63. An isolated polynucleotide encoding the antibody or antigen-binding fragment of any one of claims 1-62, or encoding a VH, a Fd, a heavy chain, a VL, and/or a light chain of the antibody or the antigen-binding fragment, wherein, optionally: (1) the heavy chain comprises or consists of SEQ ID NO:107 or SEQ ID NO:107 with the C-terminal lysine or C-terminal glycine removed; (2) the light chain comprises or consists of SEQ ID NO:108; (3) the polynucleotide comprises SEQ ID NO:109; (4) the polynucleotide comprises SEQ ID NO.: 110; (5) the polynucleotide comprises deoxyribonucleic acid (DNA) or ribonucleic acid (RNA), wherein the RNA optionally comprises messenger RNA (mRNA); and/or (6) the polynucleotide comprises a modified nucleoside, a cap-1 structure, a cap-2 structure, or any combination thereof, wherein, further optionally, the polynucleotide comprises a pseudouridine, a N6-methyladenonsine, a 5-methylcytidine, a 2-thiouridine, or any combination thereof, wherein, still further optionally, the pseudouridine comprises N1-methylpseudouridine.

64. The polynucleotide of claim 63, which is codon-optimized for expression in a host cell, wherein, optionally, the host cell comprises a human cell.

65. A recombinant vector comprising the polynucleotide of claim 63 or 64, wherein, optionally, the polynucleotide encodes: (1) SEQ ID NO:107, or SEQ ID NO:107 with the C-terminal lysine or the C-terminal glycine-lysine removed; and (2) SEQ ID NO:108, wherein, further optionally, the vector comprises SEQ ID NO:109 and SEQ ID NO:110.

66. A host cell comprising the polynucleotide of claim 63 or 64 and/or the vector of claim 65, wherein the polynucleotide is optionally heterologous to the host cell and/or wherein the host cell is capable of expressing the encoded antibody or antigen-binding fragment or polypeptide.

67. An isolated human B cell comprising the polynucleotide of claim 63 or 64 and/or the vector of claim 65, wherein polynucleotide is optionally heterologous to the human B cell and/or wherein the human B cell is immortalized.

68. A composition comprising:
(i) the antibody or antigen-binding fragment of any one of claims 1-62;
(ii) the polynucleotide of claim 63 or 64;
(iii) the recombinant vector of claim 65;
(iv) the host cell of claim 66; and/or
(v) the human B cell of claim 67,
and a pharmaceutically acceptable excipient, carrier, or diluent,
wherein, optionally:
(1) the composition comprises a first antibody or antigen-binding fragment and a second antibody or antigen-binding fragment, wherein each of the first antibody or antigen-binding fragment and the second antibody or antigen-binding fragment are different and are each according to any one of claims 1-62;
(2) the polynucleotide encodes (i) SEQ ID NO:107 or SEQ ID NO:107 with the C-terminal lysine or C-terminal glycine-lysine removed, and (ii) SEQ ID NO:108, and the polynucleotide further optionally comprises SEQ ID NO:109 and SEQ ID NO:110;
(3) the composition comprises (i) a first polynucleotide encoding SEQ ID NO.: 107 or SEQ ID NO:107 with the C-terminal lysine or C-terminal glycine-lysine removed, wherein, further optionally, the first polynucleotide comprises SEQ ID NO:109 and (ii) a second polynucleotide encoding SEQ ID NO:108, wherein, further optionally, the second polynucleotide comprises SEQ ID NO.: 110; or
(4) the composition comprises (1) a first plasmid or vector comprising a polynucleotide encoding SEQ ID NO:107 or SEQ ID NO:107 with the C-terminal lysine or C-terminal glycine-lysine removed, wherein, further optionally, the polynucleotide comprises SEQ ID NO:109, and (2) a second plasmid or vector comprising a polynucleotide encoding SEQ ID NO.: 108, wherein, further optionally, the polynucleotide comprises SEQ ID NO:110.

69. A composition comprising the polynucleotide of claim 63 or 64 or the vector of claim 65 encapsulated in a carrier molecule, wherein the carrier molecule optionally comprises a lipid, a lipid-derived delivery vehicle, such as a liposome, a solid lipid nanoparticle, an oily suspension, a submicron lipid emulsion, a lipid microbubble, an inverse lipid micelle, a cochlear liposome, a lipid microtubule, a lipid microcylinder, lipid nanoparticle (LNP), or a nanoscale platform.

70. The polynucleotide of claim 63 or 64, the vector of claim 65, or the composition of claim 68 or 69, comprising:
(1) a first polynucleotide (e.g., mRNA) encoding an antibody heavy chain comprising the VH set forth in SEQ ID NO.: 54 and a second polynucleotide (e.g., mRNA) encoding an antibody light chain comprising the VL set forth in SEQ ID NO:8; or
(2) a first polynucleotide (e.g., mRNA) encoding an antibody heavy chain comprising CDRH1, CDRH2, and CDRH3 sequences as set forth in SEQ ID NOs: (i) 55, 4, and 5, respectively, and a second polynucleotide (e.g., mRNA) encoding an antibody light chain comprising CDRL1, CDRL2, and CDRL3 sequences as set forth in SEQ ID Nos.: 9-11, respectively.

71. A method of making an antibody or antigen-binding fragment of any one of claims 1-62, comprising culturing the host cell of claim 65 or the human B cell of claim 66 for a time and under conditions sufficient for the host cell or human B cell, respectively, to express the antibody or antigen-binding fragment, wherein, optionally, the method further comprises isolating the antibody or antigen-binding fragment.

72. A method of treating or preventing an IAV infection and/or an IBV infection in a subject, the method comprising administering to the subject an effective amount of:
(i) the antibody or antigen-binding fragment of any one of claims 1-62;
(ii) the polynucleotide of any one of claims 63, 64, and 70;
(iii) the recombinant vector of claim 65 or 70;
(iv) the host cell of claim 66;
(v) the human B cell of claim 67; and/or
(vi) the composition of any one of claims 68-70.

73. The method according to claim 72, wherein: (1) the antibody or antigen-binding fragment is administered to the subject at a dose of about 3 mg/kg, about 0.9 mg/kg, or about 0.3 mg/kg; and/or (2) the IAV infection is a H5N1 and/or a H7N9 infection.

74. A method of treating or preventing an influenza infection in a human subject, the method comprising administering to the subject the polynucleotide of claim 63, 64, or 70, the recombinant vector of claim 65 or 70, or the composition of any one of claims 68-70, wherein the polynucleotide comprises mRNA, wherein, optionally, the influenza infection comprises an IAV infection and/or an IBV infection.

75. The method of any one of claims 72-74, comprising: (1) administering a single dose of the antibody or antigen-binding fragment, polypeptide, polynucleotide, recombinant vector, host cell, or composition to the subject; or (2) administering two or more doses of the antibody or antigen-binding fragment, polypeptide, polynucleotide, recombinant vector, host cell, or composition to the subject.

76. The method of any one of claims 72-75, comprising: (1) administering a dose of the antibody or antigen-binding fragment, polypeptide, polynucleotide, recombinant vector, host cell, or composition to the subject once yearly, optionally in advance of or during an influenza season; or (2) administering a dose of the antibody or antigen-binding fragment, polypeptide, polynucleotide, recombinant vector, host cell, or composition to the subject two or more times per year; e.g. about once every 6 months.

77. The method of any one of claims 72-76, comprising administering the antibody or antigen-binding fragment, polypeptide, polynucleotide, recombinant vector, host cell, or composition intramuscularly, subcutaneously, or intravenously.

78. The method of any one of claims 72-77, wherein:
(1) the treatment and/or prevention comprises post-exposure prophylaxis; and/or
(2) the subject has received, is receiving, or will receive an antiviral, wherein, optionally, the antiviral comprises a neuraminidase inhibitor, an influenza polymerase inhibitor, or both, wherein, further optionally, the antiviral comprises oseltamivir, zanamivir, baloxavir, peramivir, laninamivir, or any combination thereof.

79. The antibody or antigen-binding fragment of any one of claims 1-62, the polynucleotide of any one of claims 63, 64, and 70, the recombinant vector of claim 65 or 70, the host cell of claim 66, the human B cell of claim 67, and/or the composition of any one of claims 68-70, for use in a method of treating or preventing an IAV infection and/or an IBV infection in a subject.

80. The antibody or antigen-binding fragment of any one of claims 1-62, the polynucleotide of any one of claims 63, 64, and 70, the recombinant vector of claim 65 or 70, the host cell of claim 66, the human B cell of claim 67, and/or the composition of any one of claims 68-70, for use in the preparation of a medicament for the treatment or prevention of an IAV infection and/or an IBV infection in a subject.

81. A method for in vitro diagnosis of an IAV infection and/or an IBV infection, the method comprising:
(i) contacting a sample from a subject with an antibody or antigen-binding fragment of any one of claims 1-62; and
(ii) detecting a complex comprising an antigen and the antibody, or comprising an antigen and the antigen-binding fragment.

82. The method of any one of claims 72-78 and 81 or the antibody or antigen-binding fragment, the polypeptide, the polynucleotide, the recombinant vector, the host cell, the human B cell, and/or the composition for use of any one of claims 79 and 80, wherein:
(i) the IAV comprises a Group 1 IAV, a Group 2 IAV, or both, wherein, optionally, the Group 1 IAV NA comprises a N1, a N4, a N5, and/or a N8; and/or the Group 2 IAV NA comprises a N2, a N3, a N6, a N7, and/or a N9, wherein, further optionally, the N1 is from A/California/07/2009, is from A/California/07/2009 I223R/H275Y, is from A/California/07/2009 Q250S, is from A/Swine/Jiangsu/J004/2018, is from A/Swine/Hebei/2017, is from A/Stockholm/18/2007, is from A/Brisbane/02/2018, is from A/Michigan/45/2015, is from A/Mississippi/3/2001, is from A/Netherlands/603/2009, is from A/Netherlands/602/2009, is from A/Vietnam/1203/2004, is from A/Vietnam/1203/2004 S247R, is from A/Vietnam/1203/2004 I223R, is from A/Vietnam/1203/2004 R152I, is from A/Vietnam/1203/2004 D199N, is from A/G4/SW/Shangdong/1207/2016, is from A/G4/SW/Henan/SN13/2018, is from A/G4/SW/Jiangsu/J004/2018, is from A/Mink/Spain/2022, and/or is from A/New Jersey/8/1976; the N4 is from A/mallard duck/Netherlands/30/2011; the N5 is from A/aquatic bird/Korea/CN5/2009; the N8 is from A/harbor seal/New Hampshire/179629/2011 and/or is from A/chicken/Russia/3-29/2020; the N2 is from A/Washington/01/2007, is from A/HongKong/68, is from A/HongKong/2671/2019, is from A/HongKong/2671/2019 K431E, is from A/South Australia/34/2019, is from A/Switzerland/8060/2017, is from A/Singapore/INFIMH-16-0019/2016, is from A/Switzerland/9715293/2013, is from A/Leningrad/134/17/57, is from A/Florida/4/2006, is from A/Netherlands/823/1992, is from A/Norway/466/2014, is from is from A/Texas/50/2012, is from A/Victoria/361/2011, is from A/SW/Mexico/SG1444/2011, is from A/Aichi/2/1968, is from A/Bilthoven/21793/1972, is from A/Netherlands/233/1982, is from A/Shanghai/11/1987, is from A/Nanchang/933/1995, is from A/Fukui/45/2004, A/Brisbane/10/2007, is from A/Tanzania/205/2010, is from A/Cambodia/2020, is from A/Perth/16/2009, is from A/Kansas/14/2017, is A/Swine/Kansas/2021, is from A/Canine/Korea/VC378/2012, and/or is from A/Canine/Indiana/003018/2016; the N3 is from A/Canada/rv504/2004 and/or is from, A/chicken/Jalisco/PAVX17170/2017; the N6 is from A/swine/Ontario/01911/1/99 is from A/Ck/Suzhou/j6/2019, and/or is from A/Hangzhou/01/2021; the N7 is from A/Netherlands/078/03 and/or is from and A/Ck/621572/03; and/or the N9 is from A/Anhui/2013, is from A/Hong Kong/56/2015; and/or
(ii) the IBV NA is from: B/Lee/10/1940 (Ancestral); B/Brisbane/60/2008 (Victoria); B/Malaysia/2506/2004 (Victoria); B/Malaysia/3120318925/2013 (Yamagata); B/Wisconsin/1/2010 (Yamagata); B/Yamanashi/166/1998 (Yamagata); B/Brisbane/33/2008 (Victoria); B/Colorado/06/2017 (Victoria); B/Hubei-wujiang/158/2009 (Yamagata); B/Massachusetts/02/2012 (Yamagata); B/Netherlands/234/2011; B/Perth/211/2001 (Yamagata); B/Phuket/3073/2013 (Yamagata); B/Texas/06/2011 (Yamagata); B/HongKong/05/1972; B/Harbin/7/1994 (Victoria); B/Washington/02/2019 (Victoria); B/Perth/211/2011; B/Victoria/2/87; B/Victoria/2/87-lineage; B/Yamagata/16/88; B/Yamagata/16/88-lineage, or any combination thereof.

83. A method of treating or preventing an influenza infection in a subject, the method comprising administering to the subject an antibody or antigen-binding fragment at a dose of about 3 mg/kg, about 0.9 mg/kg, or about 0.3 mg/kg, or administering to the subject a composition comprising the antibody or antigen-binding fragment at a dose of about 3 mg/kg, about 0.9 mg/kg, or about 0.3 mg/kg, wherein:
(i) the CDRH1, CDRH2, CDRH3, CDRL1, CDRL2, and CDRL3 amino acid sequences of the antibody or antigen-binding fragment are as set forth in SEQ ID NOs: 55, 4, 5, and 9-11, respectively;
(ii) a VH of the antibody or antigen-binding fragment comprises the CDRH1, CDRH2, and CDRH3 of the VH amino acid sequence set forth in SEQ ID NO:54, and a VL of the antibody or antigen-binding fragment comprises the CDRL1, CDRL2, and CDRL3 of the VL amino acid sequence set forth in SEQ ID NO:8;
(iii) a VH and a VL of the antibody or antigen-binding fragment comprise or consist of the amino acid sequences set forth in SEQ ID NOs:54 and 8, respectively; and/or
(iv) a heavy chain and a light chain of the antibody or antigen-binding fragment comprise or consist of the amino acid sequences set forth in SEQ ID NOs: 107 (or SEQ ID NO:107 with the c-terminal lysine removed) and 108, respectively, wherein, optionally, (1) the influenza infection comprises a H5N1 IAV, a H7N9 IAV, or both; and/or
(2) the method comprises administering a single dose of the antibody or antigen-binding fragment to the subject; and/or
(3) the method comprises administering 3 mg/kg of the antibody or antigen-binding fragment to the subject, or the method comprises administering 0.9 mg/kg of the antibody or antigen-binding fragment to the subject, or the method comprises administering 0.3 mg/kg of the antibody or antigen-binding fragment to the subject.

84. The method of any one of claim 83, wherein the antibody or antigen-binding fragment comprises a human IgG1 isotype (e.g., comprising an allotype such as IgG1m3 or IgG1m17,1) and comprises M428L and N434S mutations in the Fc, and/or wherein the antibody or antigen-binding fragment comprises two heavy chains each comprising or consisting of the amino acid sequence set forth in SEQ ID NO:107 (or SEQ ID NO:107 with the C-terminal lysine removed) and two light chains each comprising or consisting of the amino acid sequence set forth in SEQ ID NO:108, and/or wherein the method comprises administering the antibody, antigen-binding fragment, or composition to the subject by intravenous administration, and/or the composition comprising the antibody or antigen-binding fragment: has an osmolality of 280-315 mOsm/kg; has no detectable endotoxin with a sensitivity <0.05 EU/mg; is sterile; has a pH of 7.4; and comprises the antibody or antigen-binding fragment purified by affinity chromatography, and/or the subject: has an H5N1 influenza infection; is at risk of contracting an H5N1 influenza infection; has been exposed to an H5N1 influenza; has an H7N9 influenza infection; is at risk of contracting an H7N9 influenza infection; and/or or has been exposed to an H7N9 influenza.

85. The method of claim 83 or 84, wherein the treating or preventing comprises prophylaxis.

86. The method of claim 83 or 84, wherein the treating or preventing comprises post-exposure prophylaxis.

87. A composition comprising an anti-NA antibody or antigen-binding fragment and a pharmaceutically acceptable carrier, excipient, or diluent, wherein:
(i) the antibody or antigen-binding fragment comprises CDRH1, CDRH2, CDRH3, CDRL1, CDRL2, and CDRL3 amino acid sequences as set forth in SEQ ID NOs:55, 4, 5, 9, 10, and 11, respectively; and/or
(ii) the antibody or antigen-binding fragment comprises the VH and VL amino acid sequences set forth in SEQ ID NOs:54 and 8, respectively, wherein, optionally, the antibody or antigen-binding fragment comprises a human IgG1 isotype (e.g., comprising an allotype such as IgG1m3 or IgG1m17,1) and comprises M428L and N434S mutations in the Fc, and/or
the antibody or antigen-binding fragment comprises the heavy chain amino acid sequence set forth in SEQ ID NO:107 (or SEQ ID NO:107 with the C-terminal lysine removed) and the light chain amino acid sequence set forth in SEQ ID NO.: 108, and/or
the antibody or antigen-binding fragment of a composition comprises two heavy chains each comprising or consisting of the amino acid sequence set forth in SEQ ID NO:107 (or SEQ ID NO:107 with the C-terminal lysine removed) and two light chains each comprising or consisting of the amino acid sequence set forth in SEQ ID NO:108, and/or the composition comprises the antibody or antigen-binding fragment at a concentration of 3 mg/kg, 0.9 mg/kg, or 0.3 mg/kg relative to the weight (kg) of a subject in need of the composition.

88. The composition of claim 87, wherein the composition: has an osmolality of 280-315 mOsm/kg; has no detectable endotoxin with a sensitivity <0.05 EU/mg; is sterile; has a pH of 7.4; and comprises the antibody or antigen-binding fragment purified by affinity chromatography.


SEQUENCE LISTING (121 sequences)

SEQ ID NO: 1 - DNA, 385 bp, synthetic construct
  source 1..385: mol_type = other DNA; note = FNI9 VH (wt-nt)
caggtccacctggtgcagtctggggctgaggtgaaggagcctgggtcctcggtgacggtctcctgcaaggcatctggaggcagcttcaacaaccaggctattagctgggtgcgacaggccccaggacaaggccttgagtggatgggagggatcttccctatctctggcacaccgaccagcgcacagaggttccagggcagagtcacatttaccgcggacgagtccacgaccacagtctacatggatctgagcagcctgagatctgacgacacggccgtctactactgtgcgagagcgggttcggattactttaatagagacctcggctgggaaaactactactttgcgtcctggggccagggaaccctggtcaccgtctcctcag

SEQ ID NO: 2 - protein, 128 aa, synthetic construct [sequence omitted]

SEQ ID NO: 3 - protein, 8 aa, synthetic construct
  source 1..8: note = FNI9 CDRH1 (aa)
GGSFNNQA

SEQ ID NO: 4 - protein, 8 aa, synthetic construct
  source 1..8: note = FNI9 CDRH2 (aa)
IFPISGTP

SEQ ID NO: 5 - protein, 21 aa, synthetic construct
  source 1..21: note = FNI9 CDRH3 (aa
ARAGSDYFNRDLGWENYYFAS

SEQ ID NO: 6 - DNA, 384 bp, synthetic construct
  source 1..384: mol_type = other DNA; note = FNI9 VH (co-nt)
caggtgcacctggtgcagagcggagctgaggtgaaggagccaggatccagcgtgacagtgtcttgcaaggcttccggcggcagcttcaacaatcaggctatctcctgggtgaggcaggctccaggacagggactggagtggatgggcggcatctttcccatctctggcacacctacctccgcccagaggttccagggaagggtgaccttcaccgctgacgagagcaccacaaccgtgtacatggatctgtcttccctgagatctgacgataccgccgtgtactattgtgccagagctggctccgactatttcaaccgcgatctgggctgggagaattactattttgcttcctggggccagggcacactggtgaccgtgagctct

SEQ ID NO: 7 - DNA, 325 bp, synthetic construct
  source 1..325: mol_type = other DNA; note = FNI9 Vk (wt-nt)
gaaatcgtgatgacgcagtctccagccaccctgtctctatcttcaggggaaagagccaccctctcctgcagggccagtcggagtgttagtagcaacttagcctggtaccagcagaaacctggccaggctcccaggctcctcatttatgatgcatccaccagggccactggtttttcagccaggttcgctggcagtgggtctgggacagagttcactctcaccatcagcagcctgcagtctgaagattctgctatttattactgtcagcagtataataactggcctccgtggacgttcggccaagggaccaaggtggaaatcaaac

SEQ ID NO: 8 - protein, 108 aa, synthetic construct
  source 1..108: note = FNI9 VK (aa)
EIVMTQSPATLSLSSGERATLSCRASRSVSSNLAWYQQKPGQAPRLLIYDASTRATGFSARFAGSGSGTEFTLTISSLQSEDSAIYYCQQYNNWPPWTFGQGTKVEIK

SEQ ID NO: 9 - protein, 6 aa, synthetic construct
  source 1..6: note = FNI9 CDRL1(aa)
RSVSSN

SEQ ID NO: 10 - skipped

SEQ ID NO: 11 - protein, 10 aa, synthetic construct [sequence omitted]

SEQ ID NO: 12 - DNA, 324 bp, synthetic construct
  source 1..324: mol_type = other DNA; note = FNI9 Vk (co-nt)
gagatcgtgatgacccagtccccagccacactgagcctgtccagcggagagagggccaccctgtcctgcagggcttcccggagcgtgtcttccaacctggcctggtaccagcagaagccaggccaggctcccagactgctgatctatgacgcctctaccagagctacaggcttctccgccaggtttgctggatctggatccggcacagagttcaccctgacaatcagctctctgcagagcgaggattctgctatctactattgtcagcagtacaacaattggcccccttggacctttggccagggcacaaaggtggagatcaag

SEQ ID NO: 13 - DNA, 384 bp, synthetic construct
  source 1..384: mol_type = other DNA; note = FNI9-VH-W110F (nt)
caggtgcacctggtgcagagcggagctgaggtgaaggagccaggatccagcgtgacagtgtcttgcaaggcttccggcggcagcttcaacaatcaggctatctcctgggtgaggcaggctccaggacagggactggagtggatgggcggcatctttcccatctctggcacacctacctccgcccagaggttccagggaagggtgaccttcaccgctgacgagagcaccacaaccgtgtacatggatctgtcttccctgagatctgacgataccgccgtgtactattgtgccagagctggctccgactatttcaaccgcgatctgggcttcgagaattactattttgcttcctggggccagggcacactggtgaccgtgagctct

SEQ ID NO: 14 - protein, 128 aa, synthetic construct
  source 1..128: note = FNI9-VH-W110F (aa)
QVHLVQSGAEVKEPGSSVTVSCKASGGSFNNQAISWVRQAPGQGLEWMGGIFPISGTPTSAQRFQGRVTFTADESTTTVYMDLSSLRSDDTAVYYCARAGSDYFNRDLGFENYYFASWGQGTLVTVSS

SEQ ID NO: 15 - protein, 21 aa, synthetic construct
  source 1..21: note = FNI9-VH-W110F CDRH3 (aa)
ARAGSDYFNRDLGFENYYFAS

SEQ ID NO: 16 - DNA, 324 bp, synthetic construct
  source 1..324: mol_type = other DNA; note = FNI9-VK-W94F (nt)
gagatcgtgatgacccagtccccagccacactgagcctgtccagcggagagagggccaccctgtcctgcagggcttcccggagcgtgtcttccaacctggcctggtaccagcagaagccaggccaggctcccagactgctgatctatgacgcctctaccagagctacaggcttctccgccaggtttgctggatctggatccggcacagagttcaccctgacaatcagctctctgcagagcgaggattctgctatctactattgtcagcagtacaacaatttccccccttggacctttggccagggcacaaaggtggagatcaag

SEQ ID NO: 17 - protein, 108 aa, synthetic construct
  source 1..108: note = FNI9-VK-W94F (aa)
EIVMTQSPATLSLSSGERATLSCRASRSVSSNLAWYQQKPGQAPRLLIYDASTRATGFSARFAGSGSGTEFTLTISSLQSEDSAIYYCQQYNNFPPWTFGQGTKVEIK

SEQ ID NO: 18 - protein, 10 aa, synthetic construct
  source 1..10: note = FNI9-VK-W94F CDRL3 (aa)
QQYNNFPPWT

SEQ ID NO: 19 - DNA, 324 bp, synthetic construct
  source 1..324: mol_type = other DNA; note = FNI9-VK-W97F (nt)
gagatcgtgatgacccagtccccagccacactgagcctgtccagcggagagagggccaccctgtcctgcagggcttcccggagcgtgtcttccaacctggcctggtaccagcagaagccaggccaggctcccagactgctgatctatgacgcctctaccagagctacaggcttctccgccaggtttgctggatctggatccggcacagagttcaccctgacaatcagctctctgcagagcgaggattctgctatctactattgtcagcagtacaacaattggccccctttcacctttggccagggcacaaaggtggagatcaag

SEQ ID NO: 20 - protein, 108 aa, synthetic construct
  source 1..108: note = FNI9-VK-W97F (aa)
EIVMTQSPATLSLSSGERATLSCRASRSVSSNLAWYQQKPGQAPRLLIYDASTRATGFSARFAGSGSGTEFTLTISSLQSEDSAIYYCQQYNNWPPFTFGQGTKVEIK

SEQ ID NO: 21 - protein, 10 aa, synthetic construct
  source 1..10: note = FNI9-VK-W97F CDRL3 (aa)
QQYNNWPPFT

SEQ ID NO: 22 - DNA, 324 bp, synthetic construct
  source 1..324: mol_type = other DNA; note = FNI9-VK-W94F-W97F (nt)
gagatcgtgatgacccagtccccagccacactgagcctgtccagcggagagagggccaccctgtcctgcagggcttcccggagcgtgtcttccaacctggcctggtaccagcagaagccaggccaggctcccagactgctgatctatgacgcctctaccagagctacaggcttctccgccaggtttgctggatctggatccggcacagagttcaccctgacaatcagctctctgcagagcgaggattctgctatctactattgtcagcagtacaacaattttccccctttcacctttggccagggcacaaaggtggagatcaag

SEQ ID NO: 23 - protein, 108 aa, synthetic construct
  source 1..108: note = FNI9-VK-W94F-W97F (aa)
EIVMTQSPATLSLSSGERATLSCRASRSVSSNLAWYQQKPGQAPRLLIYDASTRATGFSARFAGSGSGTEFTLTISSLQSEDSAIYYCQQYNNFPPFTFGQGTKVEIK

SEQ ID NO: 24 - protein, 10 aa, synthetic construct
  source 1..10: note = FNI9-VK-W94F-W97F CDRL3 (aa)
QQYNNFPPFT

SEQ ID NO: 25 - protein, 128 aa, synthetic construct
  source 1..128: note = FM08 VH
QVQLQQSGPGLVKPSQTLSLTCAISGDSVSSYNAVWNWIRQSPSRGLEWLGRTYYRSGWYNDYAESVKSRITINPDTSKNQFSLQLNSVTPEDTAVYYCARSGHITVFGVNVDAFDMWGQGTMVTVSS

SEQ ID NO: 26 - protein, 103 aa, synthetic construct
  source 1..103: note = FM08 VL
DIQMTQSPSSLSASVGDRVTITCRTSQSLSSYTHWYQQKPGKAPKLLIYAASSRGSGVPSRFSGSGSGTDFTLTISSLQPEDFATYYCQQSRTFGQGTKVEIK

SEQ ID NO: 27 - protein, 217 aa, synthetic construct
  source 1..217: note = WT hIgG1 Fc
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 19 aa, synthetic construct
  source 1..19: note = Chimeric hinge sequence
ESKYGPPCPPCPAPPVAGP

SEQ ID NO: 29 - DNA, 384 bp, synthetic construct; identical to SEQ ID NO: 6
  source 1..384: mol_type = other DNA; note = FNI9-v5-VH (co-nt)

SEQ ID NO: 30 - DNA, 324 bp, synthetic construct
  source 1..324: mol_type = other DNA; note = FNI9-v5-VK (co-nt)
gagattgtgatgacccagtcccctgctaccctgagcgtgtcccccggagagagagctaccctgagttgccgcgccagccgcagtgtctctgacaacctggcttggtaccagcagaagccaggacaggctcctaggctgctgatctatggcgcctccaccagggctacaggcatcccagctcggttctctggatccggaagcggcaccgagtttaccctgacaatctccagcctgcagagcgaggatttcgccgtgtactattgccagcattacaacatctggcctccttggacattcggtcagggaactaaagtggaaattaag

SEQ ID NO: 31 - protein, 108 aa, synthetic construct
  source 1..108: note = FNI9-v5-VK (aa)
EIVMTQSPATLSVSPGERATLSCRASRSVSDNLAWYQQKPGQAPRLLIYGASTRATGIPARFSGSGSGTEFTLTISSLQSEDFAVYYCQHYNIWPPWTFGQGTKVEIK

SEQ ID NO: 32 - protein, 6 aa, synthetic construct
  source 1..6: note = FNI9-v5-VK CDRL1 (aa)
RSVSDN

SEQ ID NO: 33 - protein, 10 aa, synthetic construct
  source 1..10: note = FNI9-v5-VK CDRL3 (aa)
QHYNIWPPWT

SEQ ID NO: 34 - protein, 330 aa, synthetic construct
  source 1..330: note = IgHG1*01, G1m3 CH1-CH3 with M428L and N434S mutations and C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 35 - protein, 107 aa, synthetic construct [sequence omitted]

SEQ ID NO: 36 - protein, 329 aa, synthetic construct
  source 1..329: note = IgHG1*01, G1m3 CH1-CH3 with M428L and N434S mutations, without C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 37 - protein, 108 aa, synthetic construct
  source 1..108: note = FNI9-variant VL
EIVMTQSPATLSVSPGERATLSCRASRSVSSNLAWYQQKPGQAPRLLIYDASTRATGIPARFSGSGSGTEFTLTISSLQSEDFAVYYCQQYNNWPPWTFGQGTKVEIK

SEQ ID NO: 38 - protein, 329 aa, synthetic construct
  source 1..329: note = IgHG1*01, G1m3 CH1-CH3 with M428L and N434A mutations, without C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 39 - protein, 458 aa, synthetic construct
  source 1..458: note = FNI9_MLNS heavy chain sequence, with C-terminal lysine
QVHLVQSGAEVKEPGSSVTVSCKASGGSFNNQAISWVRQAPGQGLEWMGGIFPISGTPTSAQRFQGRVTFTADESTTTVYMDLSSLRSDDTAVYYCARAGSDYFNRDLGWENYYFASWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 40 - protein, 458 aa, synthetic construct
  source 1..458: note = FNI9_MLNA heavy chain sequence, with C-terminal lysine
QVHLVQSGAEVKEPGSSVTVSCKASGGSFNNQAISWVRQAPGQGLEWMGGIFPISGTPTSAQRFQGRVTFTADESTTTVYMDLSSLRSDDTAVYYCARAGSDYFNRDLGWENYYFASWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 41 - protein, 215 aa, Synthetic construct
  source 1..215: note = FNI9 light chain sequence
EIVMTQSPATLSLSSGERATLSCRASRSVSSNLAWYQQKPGQAPRLLIYDASTRATGFSARFAGSGSGTEFTLTISSLQSEDSAIYYCQQYNNWPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 458 aa, synthetic construct; identical to SEQ ID NO: 39
  source 1..458: note = FNI9-variant_MLNS heavy chain sequence, with C-terminal lysine

SEQ ID NO: 43 - protein, 458 aa, synthetic construct; identical to SEQ ID NO: 40
  source 1..458: note = FNI9-variant_MLNA heavy chain sequence, with C-terminal lysine

SEQ ID NO: 44 - protein, 215 aa, synthetic construct
  source 1..215: note = FNI9-variant light chain sequence
EIVMTQSPATLSVSPGERATLSCRASRSVSSNLAWYQQKPGQAPRLLIYDASTRATGIPARFSGSGSGTEFTLTISSLQSEDFAVYYCQQYNNWPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 128 aa, synthetic construct
  source 1..128: note = FNI9-VH-FR124GL
QVQLVQSGAEVKKPGSSVKVSCKASGGSFNNQAISWVRQAPGQGLEWMGGIFPISGTPTSAQRFQGRVTFTADESTTTVYMDLSSLRSDDTAVYYCARAGSDYFNRDLGWENYYFASWGQGTLVTVSS

SEQ ID NO: 46 - protein, 128 aa, synthetic construct
  source 1..128: note = FNI9-VH.4
QVHLVQSGAEVKEPGSSVTVSCKASGGSFSNQAISWVRQAPGQGLEWMGGIFPISGTPTSAQRFQGRVTFTADESTTTVYMDLSSLRSDDTAVYYCARAGSDYFNRDLGWENYYFASWGQGTLVTVSS

SEQ ID NO: 47 - protein, 8 aa, synthetic construct
  source 1..8: note = FNI9-VH.4 CDRH1
GGSFSNQA

SEQ ID NO: 48 - protein, 128 aa, synthetic construct
  source 1..128: note = FNI9-VH.5
QVHLVQSGAEVKEPGSSVTVSCKASGGSFNKQAISWVRQAPGQGLEWMGGIFPISGTPTSAQRFQGRVTFTADESTTTVYMDLSSLRSDDTAVYYCARAGSDYFNRDLGWENYYFASWGQGTLVTVSS

SEQ ID NO: 49 - protein, 8 aa, synthetic construct
  source 1..8: note = FNI9-VH.5 CDRH1
GGSFNKQA

SEQ ID NO: 50 - protein, 128 aa, synthetic construct
  source 1..128: note = FNI9-VH.6
QVHLVQSGAEVKEPGSSVTVSCKASGGSFNNQAISWVRQAPGQGLEWMGGIFPISGTPTSAQRFQGRVTFTADESTTTVYMDLSSLRSDDTAVYYCARAGSEYFNRDLGWENYYFASWGQGTLVTVSS

SEQ ID NO: 51 - protein, 21 aa, synthetic construct
  source 1..21: note = FNI9-VH.6 CDRH3
ARAGSEYFNRDLGWENYYFAS

SEQ ID NO: 52 - protein, 128 aa, synthetic construct
  source 1..128: note = FNI9-VH.7
QVHLVQSGAEVKEPGSSVTVSCKASGGSFNNQAISWVRQAPGQGLEWMGGIFPISGTPTSAQRFQGRVTFTADESTTTVYMDLSSLRSDDTAVYYCARAGSDYFNRDLGWEQYYFASWGQGTLVTVSS

SEQ ID NO: 53 - protein, 21 aa, synthetic construct
  source 1..21: note = FNI9-VH.7 CDRH3
ARAGSDYFNRDLGWEQYYFAS

SEQ ID NO: 54 - protein, 128 aa, synthetic construct
  source 1..128: note = FNI9-VH.8
QVHLVQSGAEVKEPGSSVTVSCKASGGTFNNQAISWVRQAPGQGLEWMGGIFPISGTPTSAQRFQGRVTFTADESTTTVYMDLSSLRSDDTAVYYCARAGSDYFNRDLGWENYYFASWGQGTLVTVSS

SEQ ID NO: 55 - protein, 8 aa, synthetic construct
  source 1..8: note = FNI9-VH.8 CDRH1
GGTFNNQA

SEQ ID NO: 56 - protein, 128 aa, synthetic construct
  source 1..128: note = FNI9-VH.9
QVHLVQSGAEVKEPGSSVTVSCKASGGSFNNQAISWVRQAPGQGLEWMGGIFPISGTATSAQRFQGRVTITADESTTTVYMDLSSLRSDDTAVYYCARAGSDYFNRDLGWENYYFASWGQGTLVTVSS

SEQ ID NO: 57 - protein, 8 aa, synthetic construct
  source 1..8: note = FNI9-VH.9 CDRH2
IFPISGTA

SEQ ID NO: 58 - protein, 128 aa, synthetic construct
  source 1..128: note = FNI9-VH.10
QVHLVQSGAEVKEPGSSVTVSCKASGGSFNNQAISWVRQAPGQGLEWMGGIFPISGTPTYAQRFQGRVTITADESTTTVYMDLSSLRSDDTAVYYCARAGSDYFNRDLGWENYYFASWGQGTLVTVSS

SEQ ID NO: 59 - protein, 10 aa, synthetic construct
  source 1..10: note = FNI9-VH.10 CDRH2 and two C-terminal flanking amino acids
IFPISGTPTY

SEQ ID NO: 60 - protein, 128 aa, synthetic construct
  source 1..128: note = FNI9-VH.11
QVHLVQSGAEVKEPGSSVTVSCKASGGSFNNQAISWVRQAPGQGLEWMGGIFPISGTATYAQRFQGRVTITADESTTTVYMDLSSLRSDDTAVYYCARAGSDYFNRDLGWENYYFASWGQGTLVTVSS

SEQ ID NO: 61 - protein, 8 aa, synthetic construct
  source 1..8: note = FNI9-VH.11 CDRH2
IFPISGTA

SEQ ID NO: 62 - protein, 10 aa, synthetic construct
  source 1..10: note = FNI9-VH.11 CDRH2 and two C-terminal flanking amino acids
IFPISGTATY

SEQ ID NO: 63 - protein, 128 aa, synthetic construct
  source 1..128: note = FNI9-VH.12
QVHLVQSGAEVKEPGSSVTVSCKASGGSFNNQAISWVRQAPGQGLEWMGGIFPISGTANYAQRFQGRVTITADESTTTVYMDLSSLRSDDTAVYYCARAGSDYFNRDLGWENYYFASWGQGTLVTVSS

SEQ ID NO: 64 - protein, 10 aa, synthetic construct
  source 1..10: note = FNI9-VH.12 CDRH2 and two C-terminal flanking amino acids
IFPISGTANY

SEQ ID NO: 65 - protein, 128 aa, synthetic construct
  source 1..128: note = FNI9-VH.13
QVQLVQSGAEVKEPGSSVTVSCKASGGSFNNQAISWVRQAPGQGLEWMGGIFPISGTPTSAQRFQGRVTFTADESTTTVYMDLSSLRSDDTAVYYCARAGSDYFNRDLGWENYYFASWGQGTLVTVSS

SEQ ID NO: 66 - protein, 108 aa, synthetic construct
  source 1..108: note = FNI9-VK.7
EIVMTQSPATLSLSSGERATLSCRASRSVSSNLAWYQQKPGQAPRLLIYDASTRATGFSARFAGSGSGTEFTLTISSLQSEDSAIYYCQQYNIWPPWTFGQGTKVEIK

SEQ ID NO: 67 - protein, 10 aa, synthetic construct
  source 1..10: note = FNI9-VK.7 CDRL3
QQYNIWPPWT

SEQ ID NO: 68 - protein, 108 aa, synthetic construct
  source 1..108: note = FNI9-VK.8
EIVMTQSPATLSVSPGERATLSCRASRSVSSNLAWYQQKPGQAPRLLIYDASTRATGFSARFAGSGSGTEFTLTISSLQSEDSAIYYCQQYNNWPPWTFGQGTKVEIK

SEQ ID NO: 69 - protein, 330 aa, synthetic construct [sequence omitted]

SEQ ID NO: 70 - protein, 232 aa, synthetic construct
  source 1..232: note = Wild-type human IgG1 hinge-CH2-CH3
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 110 aa, synthetic construct
  source 1..110: note = Wild-type human IgG1 CH2
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 72 - protein, 107 aa, synthetic construct
  source 1..107: note = Wild-type human IgG1 CH3
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 125 aa, synthetic construct
  source 1..125: note = Wild-type human IgG1 hinge-CH2
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 74 - protein, 217 aa, synthetic construct
  source 1..217: note = Human IgG1 CH2-CH3 amino acid sequence with G236A, L328V, and Q295E mutations
APELLAGPSVFLFPPKPKDTLMISRTPEVTCVVVDVSHEDPEVKFNWYVDGVEVHNAKTKPREEEYNSTYRVVSVLTVLHQDWLNGKEYKCKVSNKAVPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 232 aa, synthetic construct
  source 1..232: note = Human IgG1 hinge-CH2-CH3 amino acid sequence with G236A, P230A, and Q295E mutations
EPKSCDKTHTCPPCAAPELLAGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 217 aa, synthetic construct
  source 1..217: note = Human IgG1 CH2-CH3 amino acid sequence with G236A, R292P, and I377N mutations
APELLAGPSVFLFPPKPKDTLMISRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPSRDELTKNQVSLTCLVKGFYPSDNAVEWESNGQPENNYKTTPPVLDSDGSFFLYSKLTVDKSRWQQGNVFSCSVMHEALHNHYTQKSLSLSPGK

SEQ ID NO: 77 - protein, 217 aa, synthetic construct
  source 1..217: note = Human IgG1 CH2-CH3 amino acid sequence with G236A, K334A, and Q295E mutations
APELLAGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 217 aa, synthetic construct
  source 1..217: note = Human IgG1 CH2-CH3 amino acid sequence with G236S, R292P, and Y300L mutations
APELLSGPSVFLFPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 217 aa, synthetic construct
  source 1..217: note = Human IgG1 CH2-CH3 amino acid sequence with G236A and Y300L mutations
APELLAGPSVFLFPPKPKDTLMISRTPEVTCVVVDVSHEDPEVKFNWYVDGVEVHNAKTKPREEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 217 aa, synthetic construct
  source 1..217: note = Human IgG1 CH2-CH3 amino acid sequence with G236A, R292P, and Y300L mutations
APELLAGPSVFLFPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 217 aa, synthetic construct
  source 1..217: note = Human IgG1 CH2-CH3 amino acid sequence with G236S, G420V, G446E, and L309T mutations
APELLSGPSVFLFPPKPKDTLMISRTPEVTCVVVDVSHEDPEVKFNWYVDGVEVHNAKTKPREEQYNSTYRVVSVLTVTHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQVNVFSCSVMHEALHNHYTQKSLSLSPEK

SEQ ID NO: 82 - protein, 217 aa, synthetic construct
  source 1..217: note = Human IgG1 CH2-CH3 amino acid sequence with G236A and R292P mutations
APELLAGPSVFLFPPKPKDTLMISRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 217 aa, synthetic construct
  source 1..217: note = Human IgG1 CH2-CH3 amino acid sequence with R292P and Y300L mutations
APELLGGPSVFLFPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 217 aa, synthetic construct; identical to SEQ ID NO: 82
  source 1..217: note = Human IgG1 CH2-CH3 amino acid sequence with G236A and R292P mutations

SEQ ID NO: 85 - protein, 217 aa, synthetic construct
  source 1..217: note = Human IgG1 CH2-CH3 amino acid sequence with Y300L mutation
APELLGGPSVFLFPPKPKDTLMISRTPEVTCVVVDVSHEDPEVKFNWYVDGVEVHNAKTKPREEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 217 aa, synthetic construct
  source 1..217: note = Human IgG1 CH2-CH3 amino acid sequence with E345K, G236S, L235Y, and S267E mutations
APELYSGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPRKPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 232 aa, synthetic construct
  source 1..232: note = Human IgG1 hinge-CH2-CH3 amino acid sequence with E272R, L309T, S219Y, and S267E mutations
EPKYCDKTHTCPPCPAPELLGGPSVFLFPPKPKDTLMISRTPEVTCVVVDVEHEDPRVKFNWYVDGVEVHNAKTKPREEQYNSTYRVVSVLTVTHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 217 aa, synthetic construct
  source 1..217: note = Human IgG1 CH2-CH3 amino acid sequence with G236Y mutation
APELLYGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 217 aa, synthetic construct
  source 1..217: note = Human IgG1 CH2-CH3 amino acid sequence with G236W mutation
APELLWGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 217 aa, synthetic construct
  source 1..217: note = Human IgG1 CH2-CH3 amino acid sequence with F243L, G446E, P396L, and S267E mutations
APELLGGPSVFLLPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPEK

SEQ ID NO: 91 - protein, 217 aa, synthetic construct
  source 1..217: note = Human IgG1 CH2-CH3 amino acid sequence with G236A mutation
APELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 217 aa, synthetic construct
  source 1..217: note = Human IgG1 CH2-CH3 amino acid sequence with G236A, S239D, and H268E mutations
APELLAGPDVFLFPPKPKDTLMISRTPEVTCVVVDVSEEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 330 aa, synthetic construct
  source 1..330: note = IgG1m3 CH1-CH3
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 330 aa, synthetic construct [sequence omitted]

SEQ ID NO: 95 - protein, 330 aa, synthetic construct
  source 1..330: note = IgG1m3,1 CH1-CH3
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - skipped

SEQ ID NO: 97 - DNA, 384 bp, synthetic construct
  source 1..384: mol_type = other DNA; note = FNI-UCA-IGH (wt-nt)
caggtgcagctggtgcagtctggcgccgaggtgaagaagccaggctccagcgtgaaggtgagctgcaaggcttctggcggcaccttctcttcctacgctatctcctgggtgaggcaggctccaggacagggactggagtggatgggcggcatcatccctatcttcggcacagccaactacgctcagaagtttcagggcagagtgaccatcacagccgacgagtctacctccacagcttatatggagctgagctctctgcgctccgaggataccgccgtgtactattgtgccagggctggcagcgactacttcaaccgggatctgggctgggagaattactattttgactattggggccagggcaccctggtgacagtgtccagc

SEQ ID NO: 98 - protein, 128 aa, synthetic construct
  source 1..128: note = FNI-UCA VH (aa)
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARAGSDYFNRDLGWENYYFDYWGQGTLVTVSS

SEQ ID NO: 99 - DNA, 324 bp, synthetic construct
  source 1..324: mol_type = other DNA; note = FNI-UCA-IGK (wt-nt)
gagatcgtgatgacccagtctcctgccacactgagcgtgtctccaggagagagggccaccctgtcctgcagggcttcccagagcgtgtccagcaacctggcctggtaccagcagaagccaggccaggctcccaggctgctgatctatggcgccagcaccagagctacaggcatcccagctcgcttctctggatccggaagcggcacagagtttaccctgacaatctcttccctgcagtctgaggacttcgccgtgtactattgtcagcagtacaacaattggcccccttggacctttggccagggcacaaaggtggagatcaag

SEQ ID NO: 100 - protein, 108 aa, synthetic construct
  source 1..108: note = FNI-UCA VK (aa)
EIVMTQSPATLSVSPGERATLSCRASQSVSSNLAWYQQKPGQAPRLLIYGASTRATGIPARFSGSGSGTEFTLTISSLQSEDFAVYYCQQYNNWPPWTFGQGTKVEIK

SEQ ID NO: 101 - protein, 8 aa, synthetic construct
  source 1..8: note = FNI-UCA CDRH1
GGTFSSYA

SEQ ID NO: 102 - protein, 8 aa, synthetic construct
  source 1..8: note = FNI-UCA CDRH2
IIPIFGTA

SEQ ID NO: 103 - protein, 21 aa, synthetic construct
  source 1..21: note = FNI-UCA CDRH3
ARAGSDYFNRDLGWENYYFDY

SEQ ID NO: 104 - protein, 6 aa, synthetic construct
  source 1..6: note = FNI-UCA CDRL1
QSVSSN

SEQ ID NO: 105 - skipped

SEQ ID NO: 106 - protein, 10 aa, synthetic construct [sequence omitted]

SEQ ID NO: 107 - protein, 458 aa, synthetic construct
  source 1..458: note = FNI9-v8.1 heavy chain amino acid (IgG1m3 with M428L and N434S mutations in CH3)
QVHLVQSGAEVKEPGSSVTVSCKASGGTFNNQAISWVRQAPGQGLEWMGGIFPISGTPTSAQRFQGRVTFTADESTTTVYMDLSSLRSDDTAVYYCARAGSDYFNRDLGWENYYFASWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 108 - protein, 215 aa, synthetic construct; identical to SEQ ID NO: 41
  source 1..215: note = FNI9-v8.1 light chain amino acid

SEQ ID NO: 109 - DNA, 1374 bp, synthetic construct
  source 1..1374: mol_type = other DNA; note = FNI9-v8.1 heavy chain nucleotide (IgG1m3 with M428L and N434S mutations in CH3)
caggtgcacctggtgcagagcggagctgaggtgaaggagccaggatccagcgtgacagtgtcttgcaaggcttccggcggcaccttcaacaatcaggctatctcctgggtgaggcaggctccaggacagggactggagtggatgggcggcatctttcccatctctggcacacctacctccgcccagaggttccagggaagggtgaccttcaccgctgacgagagcaccacaaccgtgtacatggatctgtcttccctgagatctgacgataccgccgtgtactattgtgccagagctggctccgactatttcaaccgcgatctgggctgggagaattactattttgcttcctggggccagggcacactggtgaccgtgagctctgcgtcgaccaagggcccatcggtcttccccctggcaccaagtagcaagagcacatccggtggcacagccgccctgggttgtctggtgaaagattatttccctgagcccgtgacagtctcctggaactctggcgccctgacctccggagtgcacacattccctgctgtgctgcagtccagcggcctgtactccctgtcttccgtggtgaccgtgccaagctcttccctgggcacccagacatatatctgcaacgtgaatcacaagccttccaatacaaaggtggacaagagggtggagccaaagagctgtgataagacccatacatgcccaccttgtccagctccagagctgctgggcggcccatccgtgttcctgtttccacccaagcccaaggacaccctgatgatctctagaaccccagaggtgacatgcgtggtggtggacgtgtcccacgaggatcccgaggtgaagtttaactggtacgtggatggcgtggaggtgcataatgctaagacaaagcccagggaggagcagtacaacagcacctatcgggtggtgtctgtgctgacagtgctgcatcaggactggctgaacggcaaggagtataagtgcaaggtgagcaataaggccctgcctgctccaatcgagaagaccatctctaaggccaagggccagcccagagagcctcaggtgtacacactgcctccaagccgcgaggagatgaccaagaaccaggtgtctctgacatgtctggtgaagggcttctatccctctgacatcgctgtggagtgggagtccaatggccagcctgagaacaattacaagaccacaccccctgtgctggactccgatggcagcttctttctgtattccaagctgaccgtggataagagcaggtggcagcagggcaacgtgttctcctgttctgtgctgcacgaagccctgcactcccattatactcagaagtccctgtccctgtcccctggaaaa

SEQ ID NO: 110 - DNA, 645 bp, synthetic construct
  source 1..645: mol_type = other DNA; note = FNI9-v8.1 light chain nucleotide
gagatcgtgatgacacagtctccagccaccctgtccctgtccagcggagagagagccacactgagctgcagggctagcaggtccgtgtcctccaacctggcctggtaccagcagaagcctggccaggctccacgcctgctgatctatgacgccagcacaagagctaccggcttctctgccaggtttgctggatccggaagcggaaccgagttcaccctgacaatcagctctctgcagagcgaggactctgctatctactattgccagcagtacaacaattggcccccttggacattcggccagggcaccaaggtggagatcaagcgtacggtggctgcaccatctgtcttcatcttcccgccatctgatgagcaattgaaatctggaactgcctccgtggtgtgcctgctgaacaatttctaccccagggaggccaaggtgcagtggaaggtggacaacgctctgcagagcggcaattctcaggagtccgtgaccgagcaggacagcaaggattctacatattccctgtccagcaccctgacactgagcaaggccgattacgagaagcacaaggtgtatgcttgtgaggtgacccatcagggcctgtcttcccctgtgacaaagtctttcaacaggggagagtgt

SEQ ID NO: 111 - protein, 458 aa, synthetic construct
  source 1..458: note = FM08_MLNS heavy chain amino acid
QVQLQQSGPGLVKPSQTLSLTCAISGDSVSSYNAVWNWIRQSPSRGLEWLGRTYYRSGWYNDYAESVKSRITINPDTSKNQFSLQLNSVTPEDTAVYYCARSGHITVFGVNVDAFDMWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 112 - protein, 210 aa, synthetic construct
  source 1..210: note = FM08 light chain amino acid
DIQMTQSPSSLSASVGDRVTITCRTSQSLSSYTHWYQQKPGKAPKLLIYAASSRGSGVPSRFSGSGSGTDFTLTISSLQPEDFATYYCQQSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - DNA, 12 bp, synthetic construct
  source 1..12: mol_type = other DNA; note = Primer Uni12
agcraaagcagg

SEQ ID NO: 114 - DNA, 19 bp, synthetic construct
  source 1..19: mol_type = other DNA; note = Forward Primer
caattggctctgtctctct

SEQ ID NO: 115 - DNA, 19 bp, synthetic construct
  source 1..19: mol_type = other DNA; note = Reverse Primer
atgaaattgatgttcgccc

SEQ ID NO: 116 - DNA, 18 bp, synthetic construct
  source 1..18: mol_type = other DNA; note = Primer
gaagagccgatactagaa

SEQ ID NO: 117 - DNA, 18 bp, synthetic construct
  source 1..18: mol_type = other DNA; note = Primer
ttctagtatcggctcttc

SEQ ID NO: 118 - DNA, 22 bp, synthetic construct
  source 1..22: mol_type = other DNA; note = MBTuni-12 Primer
acgcgtgatcagcaaaagcagg

SEQ ID NO: 119 - DNA, 23 bp, synthetic construct
  source 1..23: mol_type = other DNA; note = MBTuni-13 Primer
acgcgtgatcagtagaaacaagg

SEQ ID NO: 120 - DNA, 22 bp, synthetic construct
  source 1..22: mol_type = other DNA; note = Forward Primer
attctccttggaatttgccctt

SEQ ID NO: 121 - DNA, 21 bp, synthetic construct
  source 1..21: mol_type = other DNA; note = Reverse Primer
catagcgtaaaaggagcaaca